(19) United States
(12) Patent Application Publication
(10) Pub. No.: US 2021/0317457 A1
(43) Pub. Date: Oct. 14, 2021

(54) ANTISENSE OLIGONUCLEOTIDE (ASO) MOLECULES AND USES THEREOF FOR CORONAVIRUS DISEASES

(71) Applicant: ALIGOS THERAPEUTICS, INC., South San Francisco, CA (US)

(72) Inventors: Leonid BEIGELMAN, South San Francisco, CA (US); Antitsa STOYCHEVA, South San Francisco, CA (US); Aneerban BHATTACHARYA, South San Francisco, CA (US); David Bernard SMITH, South San Francisco, CA (US); Rajendra K. PANDEY, Foster City, CA (US); Saul Martinez MONTERO, San Bruno, CA (US); Vivek Kumar RAJWANSHI, Cupertino, CA (US); Jin HONG, Pacifica, CA (US)

(73) Assignee: ALIGOS THERAPEUTICS, INC., South San Francisco, CA (US)

(21) Appl. No.: 17/226,710

(22) Filed: Apr. 9, 2021

Related U.S. Application Data
(60) Provisional application No. 63/008,344, filed on Apr. 10, 2020.

(51) Int. Cl. C12N 15/113; A61K 45/06; A61K 31/713; A61P 31/14

(52) U.S. Cl. CPC C12N 15/1131; A61K 45/06; A61K 31/713; A61P 31/14; C12N 2310/11; C12N 2310/314; C12N 2310/351; C12N 2310/341; C12N 2310/321; C12N 2310/3231; C12N 2320/31; C12N 2310/315

(57) ABSTRACT

The present disclosure relates to the field of pharmaceutical compounds and preparations and method of their use in the treatment of disease. Described are antisense oligonucleotide (ASO) molecules, compositions containing the same, and uses thereof for treating or preventing coronavirus infections. In particular, the present disclosure provides specific ASOs that are effective against a broad spectrum of coronaviruses, and especially the β-coronaviruses, including SARS-CoV-2, the causative agent of COVID-19.

### CROSS-REFERENCE STATEMENT

This application claims priority to U.S. Provisional Application No. 63/008,344, filed Apr. 10, 2020, the disclosure of which is hereby incorporated by reference in its entirety.

### FIELD OF THE INVENTION

The present disclosure relates to the field of pharmaceutical compounds and preparations and method of their use in the treatment of disease. Described are antisense oligonucleotide (ASO) molecules, compositions containing the same, and uses thereof for treating or preventing coronavirus infections. In particular, the present disclosure provides specific ASOs that are effective against a broad spectrum of coronaviruses, and especially the β-coronaviruses, including SARS-CoV-2, the causative agent of COVID-19.

### BACKGROUND

The following discussion is merely provided to aid the reader in understanding the disclosure and is not admitted to describe or constitute prior art thereto.

Coronavirus disease 2019 (COVID-19) (also referred to as novel coronavirus pneumonia or 2019-nCoV acute respiratory disease) is an infectious disease caused by the virus severe respiratory syndrome coronavirus 2 (SARS-CoV-2) (also referred to as novel coronavirus 2019, or 2019-nCoV). The disease was first identified in December 2019 and spread globally, causing a pandemic. Symptoms of COVID-19 include fever, cough, shortness of breath, fatigue, headache, loss of smell, nasal congestion, sore throat, coughing up sputum, pain in muscles or joints, chills, nausea, vomiting, and diarrhea. In severe cases, symptoms can include difficulty waking, confusion, blueish face or lips, coughing up blood, decreased white blood cell count, and kidney failure. Complications can include pneumonia, viral sepsis, acute respiratory distress syndrome, and kidney failure.

COVID-19 is especially threatening to public health. The virus is highly contagious, and studies currently indicate that it can be spread by asymptomatic carriers or by those who are pre-symptomatic. Likewise, the early stage of the disease is slow-progressing enough that carriers do not often realize they are infected, leading them to expose numerous others to the virus. The combination of COVID-19's ease of transmission, its high rate of hospitalization of victims, and its death rate make the virus a substantial public health risk, especially for countries without a healthcare system equipped to provide supportive care to pandemic-level numbers of patients. There is not yet a vaccine or specific antiviral treatment for COVID-19 and accordingly, there is a pressing need for treatments or cures.

SARS-CoV-2 is not the only coronavirus that causes disease. It is a β-coronavirus, a genus of coronaviruses that includes other human pathogens, including SARS-CoV (the causative agent of SARS), MERS-CoV (the causative agent of MERS), and HCoV-OC43 (a causative agent of the common cold). The infectivity of these viruses, and the severity of the diseases they cause, varies widely. β-coronavirus can also manifest as zoonotic infections, spread to and from humans and animals. Additionally, non-human species such as camels, bats, tigers, non-human primates, and rabbits can be susceptible to β-coronavirus. Accordingly, there is a pressing need for treatments or cures to multiple coronaviruses.

Antisense oligonucleotides (ASOs) are short, single-stranded DNA molecules that interact with messenger RNA to prevent translation of a targeted gene. Their DNA sequence is complementary to the specific mRNA target; binding leads to degradation of the DNA sequences with failure of protein production. Antisense oligonucleotides can be designed for many RNA sequences, so they have the potential to be useful as therapeutics for diverse diseases. Accordingly, ASO therapy has been proposed for the treatment of metabolic diseases, neurodegenerative diseases, cancer, and pathogenic infections.

The present disclosure provides ASO molecules useful against coronaviruses, and especially SARS-CoV-2, the causative agent of COVID-19 in humans. Accordingly, the present disclosure fulfills the need in the art for compounds that can be safely and effectively treat or prevent coronavirus infections in humans.

### SUMMARY

Described herein are antisense oligonucleotide (ASO) molecules and methods of using the same to treat or prevent a disease, such as a coronavirus infection.

In one aspect, the present disclosure provides an antisense oligonucleotide (ASO) comprising a 5′-wing region (A′), a central region (B′), and a 3′-wing region (C′) that together comprise 8 to 22 total nucleotide units, wherein: (a) a central region (B′) comprising 6 or more contiguous nucleotide units, (b) a 5′-wing region (A′) comprising 2 to 6 locked nucleotide units, 2′ substituted nucleotide units, or a combination thereof, and (c) a 3′-wing region (C′) comprising 2 to 6 locked nucleotide units, 2′ substituted nucleotide units, or a combination thereof; and wherein the ASO is complementary or hybridizes to a viral target RNA sequence in a coronavirus.

In some embodiments, the 5′-wing region of the ASO comprises 2 to 6 phosphorothioate-linked locked nucleosides. In some embodiments, the 3′-wing region of the ASO comprises 2 to 6 phosphorothioate-linked locked nucleosides. In some embodiments, the central region of the ASO comprises at least 5 contiguous phosphorothioate-linked DNA nucleosides.

In some embodiments, the central region of the ASO comprises 8 to 10 contiguous phosphorothioate-linked DNA nucleosides. In some embodiments, the locked nucleosides are selected from LNA, scpBNA, AmNA (N—H), AmNA (N-Me), GuNA, GuNA (N-R) where R is selected from Me, Et, i-Pr, t-Bu and combinations thereof.

In some embodiments, the 3′-wing region and the 5′-wing region comprise the same number of nucleotide units. In some embodiments, the 3′-wing region and the 5′-wing region comprise 3 nucleotide units and the central region comprises 10 nucleotide units. In some embodiments, the 3′-wing region and the 5′-wing region comprise 4 nucleotide units and the central region comprises 10 nucleotide units.

In some embodiments, the 3′-wing region and the 5′-wing region comprise a different number of nucleotide units. In some embodiments, the 3′-wing region comprises 3 nucleotide units, the 5′-wing region comprises 5 nucleotide units, and the central region comprises 10 nucleotide units.

In some embodiments, the ASO may further comprise one or more conjugation moieties.

In some embodiments, one T in the central region is replaced by (2s)T, one C in the central region is replaced by (5OH)C, one A is replaced by (8NH)A in the central region, and/or one G in the central region is replaced by (8NH)G.

In some embodiments, the 5′-wing region and/or the 3′-wing region comprises one or two ScpBNA, AmNA, or GuNA.

In some embodiments, the 5′-wing region and/or the 3′-wing region comprises a mA or mU appended to the end of the sequence. In some embodiments, the mA or mU is linked to a conjugation moiety.

In some embodiments, the ASO comprises 14-22 nucleotide units. For example, in some embodiments, the ASO comprises 16, 17, 18, or 20 nucleotide units.

In some embodiments, the viral target RNA sequence encodes a non-structural protein (nsp). In some embodiments, the nsp is selected from the group consisting of nsp8, nsp9, nsp10, nsp11, nsp12, nsp13, nsp14, and nsp15.

In some embodiments, the ASO comprises at least 8 nucleotide units from any one of SEQ ID NOs: 1-5534.

In some embodiments, the ASO comprises a sequence of any one of SEQ ID NOs: 1-5534.

In some embodiments, the ASO is at least about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or about 100% identical or complementary to the viral target RNA sequence.

In some embodiments, the ASO comprises a sequence that is at least about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or about 100% identical or complementary to at least 8 consecutive nucleotide units of GenBank Accession NC_045512.2. In some embodiments, the ASO comprises a sequence that is at least about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or about 100% identical or complementary to at least 16 consecutive nucleotide units of GenBank Accession NC 045512.2.

In some embodiments, the ASO comprises a sequence that is at least about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or about 100% identical or complementary to at least 8 consecutive nucleotide units of any one of the sequences listed in Table 2. In some embodiments, the ASO comprises a sequence that is at least about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or about 100% identical or complementary to at least 16 consecutive nucleotide units of any one of the sequences listed in Table 2.

In another aspect, the present disclosure provides an antisense oligonucleotide (ASO) comprising 14-22 nucleotide units, wherein the ASO comprises:

(a) a central region (B′) comprising 6 or more contiguous DNA nucleosides, wherein at least one of the contiguous DNA nucleosides is a modified nucleotide,
(b) a 5′-wing region (A′) comprising 2 to 6 locked nucleosides, 2′ substituted nucleosides, or a combination thereof, and
(c) a 3′-wing region (C′) comprising 2 to 6 locked nucleosides, 2′ substituted nucleosides, or a combination thereof,
wherein the ASO is complementary or hybridizes to a viral target RNA sequence in a coronavirus.

In some embodiments, the modified nucleotide occurs at the nucleotide at position 3 from the 5′ end of the central region.

In some embodiments, the modified nucleotide is a nucleotide having a protected or unprotected version of

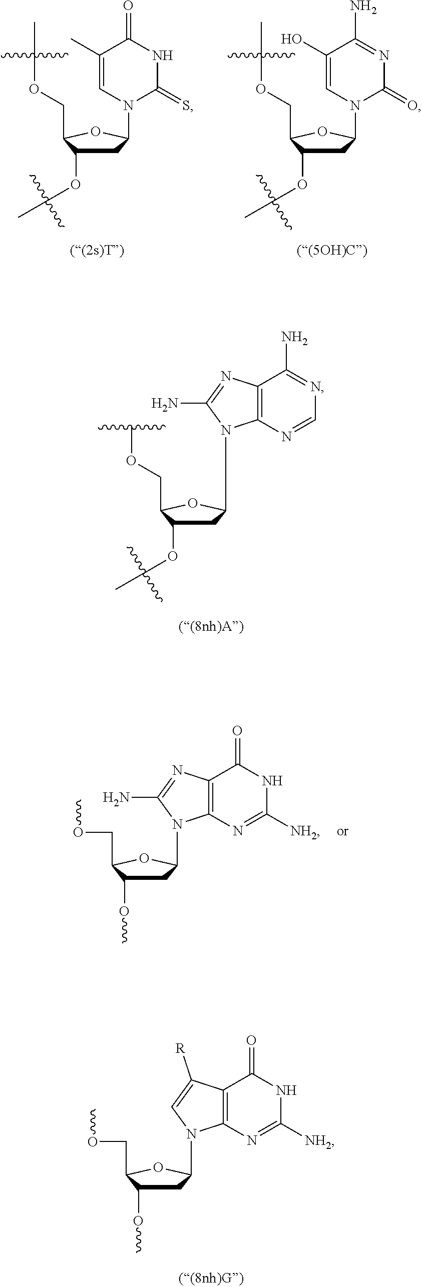

wherein:

R is a halogen or R′—C≡C—; and
R′ is C₆₋₁₂ aryl, 5- to 12-membered heteroaryl, hydroxy-C₁₋₆ alkyl, or C₁₋₇ alkanoyloxy.

In some embodiments, the modified nucleotide is a nucleotide having the structure of:

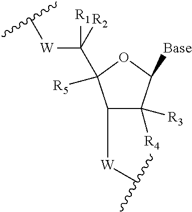

wherein:
W is independently O, N, or S;
R₁, R₂, and R₅ are independently H or D or CH₃ or CD₃;

### R₃ is H or F;

R₄ is F or OCH₃ or OCD₃; and

### Base is

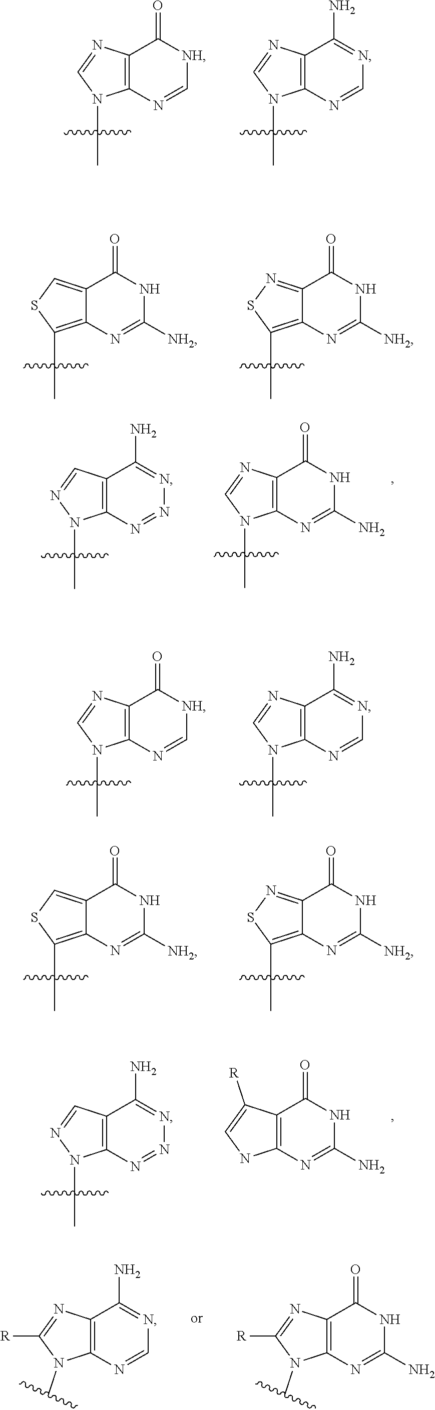

wherein:
R is a halogen or R′—C≡C—; and
R′ represents C₆₋₁₂ aryl, 5- to 12-membered heteroaryl, hydroxy-C₁₋₆ alkyl, or C₁₋₇ alkanoyloxy.

In some embodiments, the ASO comprises a nucleotide sequence that is at least 90% identical to a nucleotide sequence selected from the sequences listed in Table 1 or 2. In some embodiments, the viral target RNA sequence encodes a non-structural protein (nsp). In some embodiments, the nsp is selected from the group consisting of nsp8, nsp9, nsp10, nsp11, nsp12, nsp13, nsp14, and nsp15. In some embodiments, the ASO is at least about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or about 100% identical or complementary to the viral target RNA sequence.

In some embodiments of the foregoing aspects, the coronavirus is an α-coronavirus or a β-coronavirus. In some embodiments, the coronavirus is selected from the group consisting of CoV 229E, CoV NL63, CoV OC43, CoV HKU1, Middle East Respiratory Syndrome (MERS)-CoV, Severe Acute Respiratory Syndrome (SARS)-CoV, and SARS-CoV-2.

In another aspect, the present disclosure provides an antisense oligonucleotide (ASO) comprising any one of SEQ ID NOs: 5539-5718 In some embodiments, the ASO of claim 28, wherein the ASO is selected from ASO-143 (SEQ ID NO: 5682), ASO-144 (SEQ ID NO: 5683), ASO-145 (SEQ ID NO: 5684), ASO-146 (SEQ ID NO: 5685), ASO-147 (SEQ ID NO: 5686), ASO-148 (SEQ ID NO: 5687), ASO-149 (SEQ ID NO: 5688), ASO-150 (SEQ ID NO: 5689), ASO-151 (SEQ ID NO: 5690), ASO-152 (SEQ ID NO: 5691), ASO-153 (SEQ ID NO: 5692), ASO-154 (SEQ ID NO: 5693), ASO-155 (SEQ ID NO: 5694), ASO-156 (SEQ ID NO: 5695), ASO-158 (SEQ ID NO: 5697), ASO-159 (SEQ ID NO: 5698), ASO-160 (SEQ ID NO: 5699), ASO-163 (SEQ ID NO: 5702), ASO-164 (SEQ ID NO: 5703), ASO-165 (SEQ ID NO: 5704), ASO-166 (SEQ ID NO: 5705), ASO-167 (SEQ ID NO: 5706), ASO-168 (SEQ ID NO: 5707), ASO-169 (SEQ ID NO: 5708), ASO-170 (SEQ ID NO: 5709), ASO-171 (SEQ ID NO: 5710), ASO-172 (SEQ ID NO: 5711), ASO-173 (SEQ ID NO: 5712), ASO-174 (SEQ ID NO: 5713), ASO-175 (SEQ ID NO: 5714), ASO-176 (SEQ ID NO: 5715), ASO-177 (SEQ ID NO: 5716), ASO-178 (SEQ ID NO: 5717), and ASO-179 (SEQ ID NO: 5718).

In another aspect, the present disclosure provides an ASO of any of the foregoing embodiments for use in treatment or prevention of a disease. In some embodiments, the disease is a coronavirus infection.

In another aspect, the present disclosure provides a pharmaceutical composition comprising one or more of the ASOs of any of the foregoing aspects and a pharmaceutically acceptable carrier. In some embodiments, the pharmaceutical composition is for use in the treatment or prevention of a disease. In some embodiments, the disease is a coronavirus infection.

In another aspect, the present disclosure provides a method of treating or preventing a disease, comprising administering to a subject in need thereof a therapeutically effective amount of one or more of the ASOs of any one of the foregoing aspects or the pharmaceutical composition of any one of the foregoing aspects.

In some embodiments of the disclosed methods, the disease is a viral disease. In some embodiments, the viral disease is caused by an RNA virus. In some embodiments, the RNA virus is a single-stranded RNA virus (ssRNA virus). In some embodiments, the ssRNA virus is a positive-sense single-stranded RNA virus ((+)ssRNA virus). In some embodiments, (+)ssRNA virus is a coronavirus. In some embodiments, the coronavirus is a β-coronavirus.

In some embodiments of the disclosed methods, the disease is a coronavirus infection selected from the group consisting of Middle East Respiratory Syndrome (MERS), Severe Acute Respiratory Syndrome (SARS), and COVID-19.

In some embodiments of the disclosed methods, the subject has been treated with one or more additional coronavirus treatment agents and/or antiviral agents. In some embodiments of the disclosed methods, the subject is concurrently treated with one or more additional coronavirus treatment agents and/or antiviral agents.

In some embodiments of the disclosed methods, the subject is a human, for example a human subject that is 60 years old or older. In some embodiments of the disclosed methods, the subject is a non-human primate. In some embodiments of the disclosed methods, the subject is a cat. In some embodiments of the disclosed methods, the subject is a camel.

In some embodiments of the disclosed methods, the disease causes one or more symptoms selected from coughing, sore throat, runny nose, sneezing, headache, fever, shortness of breath, myalgia, abdominal pain, fatigue, difficulty breathing, persistent chest pain or pressure, difficulty waking, loss of smell and taste, muscle or joint pain, chills, nausea or vomiting, nasal congestion, diarrhea, haemoptysis, conjunctival congestion, sputum production, chest tightness, and palpitations. In some embodiments of the disclosed methods, the disease can cause complications selected from sinusitis, otitis media, pneumonia, acute respiratory distress syndrome, disseminated intravascular coagulation, pericarditis, and kidney failure.

In another aspect, the present disclosure provides a use of one or more of the ASOs of any one of claims 1-29 for the manufacture of a medicament for treating or preventing a disease. In some embodiments, the disease is a coronavirus infection.

In some embodiments of the disclosed methods and uses, the ASO is administered intravenously, subcutaneously, or via inhalation.

The foregoing general description and following detailed description are exemplary and explanatory and are intended to provide further explanation of the disclosure as claimed. Other objects, advantages, and novel features will be readily apparent to those skilled in the art from the following brief description of the drawings and detailed description of the disclosure.

### BRIEF DESCRIPTION OF THE DRAWINGS

FIG. 1 (FIG. 1) shows the coronaviridae family and its four genera (top panel) and the full-length genome of NCBI 407 (bottom panel), which encodes 28 proteins across multiple open reading frames (ORFs).

FIG. 2 (FIG. 2) shows the percent identity between multiple coronavirus, including sudden acute respiratory syndrome coronavirus (SARS-CoV), Middle East respiratory syndrome coronavirus (MERS-CoV), and human coronavirus OC43 (top panel), and an alignment of the highly similar region of the genomes encodings non-structural protein 8 (nsp8) to non-structural protein (nsp15) (bottom panel).

FIG. 3 (FIG. 3) shows details of nsp8-nsp15.

### DETAILED DESCRIPTION

Disclosed herein are antisense oligonucleotide (ASO) molecules. The disclosed ASO molecules comprise a 5′-wing region (A′), a central region (B′), and a 3′-wing region (C′) that together comprise 8 to 20 total nucleotide units, wherein: (a) a central region (B′) comprising 6 or more contiguous nucleotide units, (b) a 5′-wing region (A′) comprising 2 to 6 locked nucleotide units or 2′ substituted nucleotide units, and (c) a 3′-wing region (C′) comprising 2 to 6 locked nucleotide units or 2′ substituted nucleotide units; and wherein the ASO is complementary or hybridizes to a viral target RNA sequence in a coronavirus. In some embodiments, the sequence of the ASO molecules may comprise one or more modified, lock, conjugated, or linked nucleotide units, and the ASO may comprise a targeting moiety to direct its uptake in a particular cell type.

Further disclosed herein are pharmaceutical compositions comprising one or more of the disclosed ASO molecules and a pharmaceutically acceptable carrier or diluent.

Further disclosed herein is a method for treating a disease in a subject in need thereof, comprising administering the subject one or more ASOs or pharmaceutical compositions of any of the embodiments described herein. In some embodiments, the disease is a viral infection, such as a coronavirus infection.

Further disclosed herein is the use of one or more ASOs according to any of the embodiments described herein in the manufacture of a medicament for treating a disease, such as a viral infection or, more specifically, a coronavirus infection.

Further disclosed herein is a method of treating a β-coronavirus-caused disease in a subject in need thereof, comprising administering the subject one or more ASOs according to any of the embodiments described herein.

Exemplary siNA, which may be used to treat and/or prevent coronavirus infections (e.g., COVID-19) are also described herein.

### Definitions

It is to be understood that methods are not limited to the particular embodiments described, and as such may vary. It is also to be understood that the terminology used herein is for the purpose of describing particular embodiments only and is not intended to be limiting. The scope of the present technology will be limited only by the appended claims.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, representative illustrative methods and materials are now described.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges and are also encompassed within the invention, subject to any specifically excluded limit in the stated range. Where the stated range includes one or both of the limits, ranges excluding either or both of those included limits are also included in the invention.

As used in the specification and claims, the singular form “a,” “an” and “the” include singular and plural references unless the context clearly dictates otherwise.

As used herein, the term “comprising” is intended to mean that the compositions and methods include the recited elements, but not excluding others. “Consisting essentially of” when used to define compositions and methods, shall mean excluding other elements of any essential significance to the composition or method. “Consisting of” shall mean excluding more than trace elements of other ingredients for claimed compositions and substantial method steps. Embodiments defined by each of these transition terms are within the scope of this disclosure. Accordingly, it is intended that the methods and compositions can include additional steps and components (comprising) or alternatively including steps and compositions of no significance (consisting essentially of) or alternatively, intending only the stated method steps or compositions (consisting of).

As used herein, “about” means plus or minus 10% as well as the specified number. For example, “about 10” should be understood as both “10” and “9-11.”

As used herein, “optional” or “optionally” means that the subsequently described event or circumstance may or may not occur, and that the description includes instances where said event or circumstance occurs and instances where it does not.

As used here, “activated group” means a chemical moiety with a high reactivity toward a particular chemical functional group, sometimes called a “reactive group” or a “reactive end,” under particular conditions. Examples of activated groups include phosphoramidite groups, which are known to be highly reactive toward nucleophiles, for instance in the form of unprotected hydroxyl groups, in the presence of weak acids. Phosphoramidite groups are well-known, for example, in solid-phase oligonucleotide synthesis. A phosphoramidite group allowed to react with a nucleophile of another moiety under weakly acidic conditions will displace the secondary amine substituent in favor of a bond between the nucleophile and the phosphorus atom of the phosphoramidite group. An example of a phosphoramidite group in some embodiments here is represented by the following structure, i.e., cyanoethoxy N,N-diisopropylphoramidite:

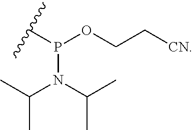 [SMILES: *P(OCCC#N)N(C(C)C)C(C)C]

As used here, “linker” means a chemical moiety that connects two other chemical moieties. For the purposes of the present disclosure, one end of a linker may be attached to an oligonucleotide, while the other end is attached to a targeting moiety (e.g., a phosphorus atom between the oligonucleotide and a conjugated moiety).

The terms “individual,” “subject,” and “patient” are used interchangeably herein, and refer to any individual mammal, e.g., bovine, canine, feline, equine, simian, porcine, camelid, bat, or human, being treated according to the disclosed methods or uses. In preferred embodiments, the subject is a human.

As used herein, the phrases “effective amount,” “therapeutically effective amount,” and “therapeutic level” mean the ASO dosage or concentration in a subject that provides the specific pharmacological effect for which the ASO is administered in a subject in need of such treatment, i.e. to treat or prevent a coronavirus infection (e.g., MERS, SARS, or COVID-19). It is emphasized that a therapeutically effective amount or therapeutic level of an ASO will not always be effective in treating the infections described herein, even though such dosage is deemed to be a therapeutically effective amount by those of skill in the art. For convenience only, exemplary dosages, drug delivery amounts, therapeutically effective amounts, and therapeutic levels are provided below. Those skilled in the art can adjust such the amount in accordance with standard practices as needed to treat a specific subject and/or condition. The therapeutically effective amount may vary based on the route of administration and dosage form, the age and weight of the subject, and/or the subject's condition, including the type and severity of the coronavirus infection.

The terms “treatment” or “treating” as used herein with reference to a coronavirus infections refer to reducing or eliminating viral load and/or improving or ameliorating one or more symptoms of an infection such as cough, shortness of breath, body aches, chills, and/or fever.

The terms “prevent” or “preventing” as used herein with reference to a coronavirus infections refer to precluding an infection from developing in a subject exposed to a coronavirus and/or avoiding the development of one or more symptoms of an infection such as cough, shortness of breath, body aches, chills, and/or fever. “Prevention” may occur when the viral load is never allowed to exceed beyond a threshold level at which point the subject begins to feel sick or exhibit symptoms. “Prevention” may also, in some embodiments, refer to the prevention of a subsequent infection once an initial infection has been treated or cured.

As used herein, the term “pharmaceutical composition” refers to the combination of an active agent with a carrier, inert or active, making the composition especially suitable for diagnostic or therapeutic use in vivo or ex vivo.

As used herein, the term “pharmaceutically acceptable carrier” refers to any of the standard pharmaceutical carriers, such as a phosphate buffered saline solution, water, emulsions (e.g., such as an oil/water or water/oil emulsions), and various types of wetting agents. The compositions also can include stabilizers and preservatives. For examples of carriers, stabilizers and adjuvants, see, for example, Martin, Remington's Pharmaceutical Sciences, 15th Ed., Mack Publ. Co., Easton, Pa. [1975].

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal and intrasternal injection and infusion.

The phrases “systemic administration,” “administered systemically,” “peripheral administration” and “administered peripherally” as used herein mean the administration of a compound, drug or other material other than directly into the central nervous system, such that it enters the patient's system and, thus, is subject to metabolism and other like processes, for example, subcutaneous administration.

The target gene may be any gene in a cell or virus. Here, “target gene” and “target sequence” are used synonymously.

Here, “conjugation moiety” and “target moiety” or “targeting moiety” are used synonymously.

As used herein, the term “nucleotide unit” refers to an individual unit of an ASO including, but not limited to, a nucleotide, a nucleoside, a modified nucleotide, or a locked nucleotide.

As used herein, the term “nucleobase” refers to a nitrogen-containing biological compound that forms a nucleoside. Examples of nucleobases include, but are not limited to, thymine, uracil, adenine, cytosine, guanine, aryl, heteroaryl, and an analogue or derivative thereof.

As used herein, “modified nucleotide” includes any nucleic acid or nucleic acid analogue residue that contains a modification or substitution in the chemical structure of an unmodified nucleotide base, sugar (including, but not limited to, 2′-substitution), or phosphate (including, but not limited to, alternate internucleotide linkers, such as phosphorothioates or the substitution of bridging oxygens in phosphate linkers with bridging sulfurs), or a combination thereof. Non-limiting examples of modified nucleotides are shown herein.

For the purposes of the present disclosure, a DNA sequence that replaces all the U residues of an RNA sequence with T residues is “identical” to the RNA sequence, and vice versa. Accordingly, a sequence that is “identical to an RNA corresponding to” a DNA sequence constitutes the DNA sequence with all T replaced by U.

Throughout the description, where compositions are described as having, including, or comprising specific components, or where processes and methods are described as having, including, or comprising specific steps, it is contemplated that, additionally, there are compositions of the present disclosure that consist essentially of, or consist of, the recited components, and that there are processes and methods according to the present disclosure that consist essentially of, or consist of, the recited processing steps.

As a general matter, compositions specifying a percentage are by weight unless otherwise specified. Further, if a variable is not accompanied by a definition, then the previous definition of the variable controls.

### Coronaviruses and Coronavirus Infections

The antisense oligonucleotide (ASO) molecules and compositions described herein may be administered to a subject to treat or prevent a disease in a subject in need thereof, in particular when the disease is a viral infection, such as a coronavirus infection. Further disclosed herein are uses of any of the ASO molecules or compositions disclosed herein in the manufacture of a medicament for treating or preventing a viral infection, such as a coronavirus infection.

In some embodiments of the disclosed method and uses, the disease being treated is a viral disease. In some embodiments, the viral disease is caused by an RNA virus. In some embodiments, the RNA virus is a single-stranded RNA virus (ssRNA virus). In some embodiments, the ssRNA virus is a positive-sense single-stranded RNA virus ((+)ssRNA virus). In some embodiments, the (+)ssRNA virus is a coronavirus.

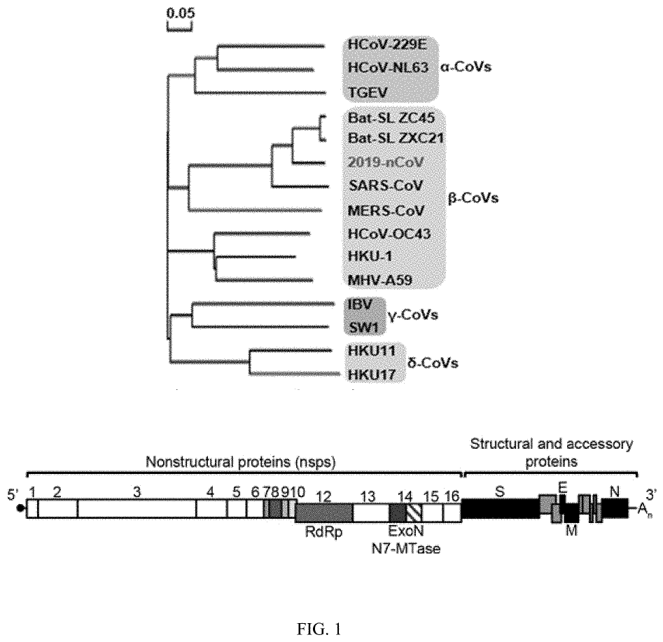

Coronaviruses are a family of viruses (i.e., the coronaviridae family) that cause respiratory infections in mammals and that comprise a genome that is roughly 30 kilobases in length. The coronaviridae family is divided into four genera and the genome encodes 28 proteins across multiple open reading frames, including 16 non-structural proteins (nsp) that are post-translationally cleaved from a polyprotein (see FIG. 1).

The coronaviridae family includes both α-coronaviruses or β-coronaviruses, which both mainly infect bats, but can also infect other mammals like humans, camels, and rabbits. β-coronaviruses have, to date, been of greater clinical importance, having caused epidemics including severe acute respiratory syndrome (SARS), Middle East respiratory syndrome (MERS), and COVID-19. Other disease-causing β-coronaviruses include OC44, and HKU1. Non-limiting examples of disease-causing α-coronaviruses include, but are not limited to, 229E and NL63.

In some embodiments, the coronavirus is a β-coronaviruses. In some embodiments, the β-coronaviruses is selected from the group consisting of severe acute respiratory syndrome coronavirus 2 (SARS-CoV-2) (also known by the provisional name 2019 novel coronavirus, or 2019-nCoV), human coronavirus OC43 (hCoV-OC43), Middle East respiratory syndrome-related coronavirus (MERS-CoV, also known by the provisional name 2012 novel coronavirus, or 2012-nCoV), and severe acute respiratory syndrome-related coronavirus (SARS-CoV, also known as SARS-CoV-1). In some embodiments, the β-coronaviruses is SARS-CoV-2, the causative agent of COVID-19.

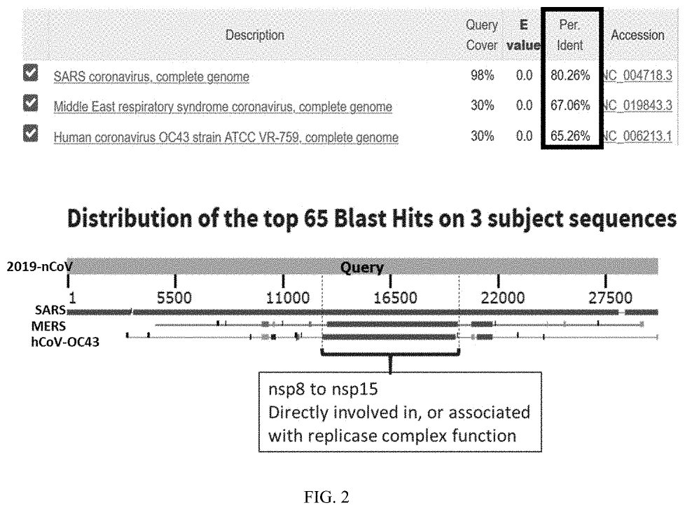

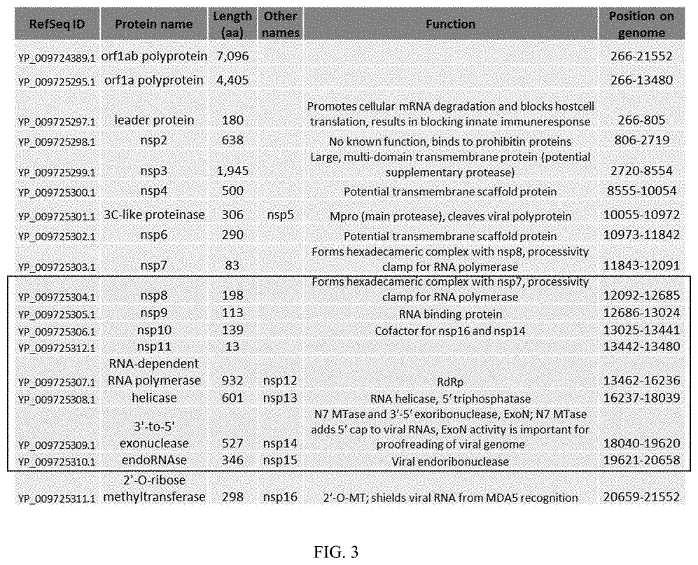

As shown in FIGS. 2 and 3, several disease-causing coronaviruses share a high degree of homology in the regions of the genome encoding non-structural proteins (nsp), and more specifically, in the region encoding nsp8-nsp15. Indeed, there is roughly 65% identity across the roughly 7 kB sequence of β-coronaviruses from about nucleotide 12900 to about nucleotide 19900 of 2019-nCoV, and some subsections of the genomic span of nsp8 to nsp15 may comprise 95% or more identity. All of the genes in this region encode non-structural proteins associated with replication. Accordingly, this segment of the genome is suitable for targeting with an ASO that can provide a broad spectrum treatment for multiple different types of coronavirus, such as MERS-CoV, SARS-CoV-1, and SARS-CoV-2.

Without wishing to be bound by theory, upon entry into a cell, any of the ASO molecules disclosed herein may interact with proteins in the cell to form a RNA-Induced Silencing Complex (RISC). Once the ASO is part of the RISC, the ASO may bind to a complementary messenger RNA (mRNA) from the virus, which results in silencing of the gene that encodes the mRNA.

In some embodiments, the target gene is a viral gene. In some embodiments, the viral gene is from an RNA virus. In some embodiments, the RNA virus is a single-stranded RNA virus (ssRNA virus). In some embodiments, the ssRNA virus is a positive-sense single-stranded RNA virus ((+)ssRNA virus). In some embodiments, the (+)ssRNA virus is a coronavirus. In some embodiments, the coronavirus is a β-coronaviruses. In some embodiments, the β-coronaviruses is severe acute respiratory syndrome coronavirus 2 (SARS-CoV-2) (also known by the provisional name 2019 novel coronavirus, or 2019-nCoV), human coronavirus OC43 (hCoV-OC43), Middle East respiratory syndrome-related coronavirus (MERS-CoV, also known by the provisional name 2012 novel coronavirus, or 2012-nCoV), severe acute respiratory syndrome-related coronavirus (SARS-CoV, also known as SARS-CoV-1). In some embodiments, the β-coronaviruses is SARS-CoV-2.

In some embodiments, the target gene is selected from genome of SARS-CoV-2. In some embodiments, SARS-CoV-2 has a genome sequence shown in the nucleotide sequence of SEQ ID NO: 2407, which corresponds to the nucleotide sequence of GenBank Accession No. NC_045512.2, which is incorporated by reference in its entirety.

In some embodiments, the target gene is selected from genome of SARS-CoV. In some embodiments, SARS-CoV has a genome sequence shown in the nucleotide sequence of SEQ ID NO: 2408, which corresponds to the nucleotide sequence of GenBank Accession No. NC 004718.3, which is incorporated by reference in its entirety.

In some embodiments, the target gene is selected from the genome of MERS-CoV. In some embodiments, MERS-CoV has a genome sequence shown in the nucleotide sequence of SEQ ID NO: 2409, which corresponds to the nucleotide sequence of GenBank Accession No. NC_019843.3, which is incorporated by reference in its entirety.

In some embodiments, the target gene is selected from the genome of hCoV-OC43. In some embodiments, hCoV-OC43 has a genome sequence shown in the nucleotide sequence of SEQ ID NO: 2410, which corresponds to the nucleotide sequence of GenBank Accession No. NC_006213.1, which is incorporated by reference in its entirety.

### Antisense Oligonucleotide (ASO) Molecules

Compounds of the present disclosure include modified antisense oligonucleotides (ASOs). In some embodiments, the ASO comprises 8-22 nucleotide units, e.g., 8, 9, 10, 11, 12, 13, 14, 15, 16, 17, 18, 19, 20, 21, or 22 nucleotide units. In some embodiments, the ASO is a gapmer ASO that comprises three regions: a 5′-wing region (A′) comprising modified nucleotides; a central region (B′) comprising nucleotides of a different type from the wings, e.g., nucleotides capable of inducing RNase H cleavage; and a 3′-wing region (C′) comprising modified nucleotides.

In some embodiments, the 5′-wing region and the 3′-wing region comprise 2-6 nucleotides, e.g., 2, 3, 4, 5, or 6 nucleotides. One or more of these nucleotides is modified (e.g., 1, 2, 3, 4, 5, or 6 of the nucleotides is modified). In some embodiments, the central region may comprise 6 or more contiguous DNA nucleosides, linked by phosphodiester or thiophosphate (“ps”) internucleotide linkages. In some embodiments, the central region includes one or more modified nucleotide. For example, the central region may include one or more modified nucleotide where the central region is capable of inducing RNase H cleavage. In some embodiments, the central region includes one or more modified nucleotide having a modified nucleobase. In some embodiments, the central region comprises 3, 4, 5, 6, 7, 8, 9, 10, or 11 contiguous DNA nucleosides. In some embodiments, 1, 2, 3, 4, 5, 6, 7, 8, 9, 10, or 11 of the DNA nucleosides in the central region are modified.

Thus, in some aspects, the gapmer ASO compounds of the disclosure include compounds of formula (I):

A′-B′-C′,

wherein A′ and C′ each independently comprise 2-6 nucleotides, with one or more being a modified nucleotide, B′ comprises 6 or more contiguous DNA nucleosides linked by phosphodiester or thiophosphate internucleotide linkages. In some embodiments, A′ and C′ comprise the same number of nucleotides and/or nucleosides. In some embodiments, A′ and C′ comprise different numbers of nucleotides and/or nucleosides. In some embodiments, B′ comprises one or more modified DNA nucleosides. In some embodiments, the modified nucleotide is selected from locked nucleosides or 2′-substituted nucleosides. In some embodiments, the modified DNA nucleoside is selected from locked nucleosides or 2′-substituted nucleosides.

In some embodiments, the number of nucleotides and/or nucleosides in A′, B′, and C′ are selected from the following group (A′:B′:C′): (2:10:2), (2:10:3), (2:10:4), (2:10:5), (3:10:2), (3:10:3), (3:10:4), (3:10:5), (4:10:2), (4:10:3), (4:10:4), (4:10:5), (5:10:2), (5:10:3), (5:10:4), (5:10:5), (2:9:2), (2:9:3), (2:9:4), (2:9:5), (3:9:2), (3:9:3), (3:9:4), (3:9:5), (4:9:2), (4:9:3), (4:9:4), (4:9:5), (5:9:2), (5:9:3), (5:9:4), (5:9:5), (2:8:2), (2:8:3), (2:8:4), (2:8:5), (3:8:2), (3:8:3), (3:8:4), (3:8:5), (4:8:2), (4:8:3), (4:8:4), (4:8:5), (5:8:2), (5:8:3), (5:8:4), (5:8:5), (2:7:2), (2:7:3), (2:7:4), (2:7:5), (3:7:2), (3:7:3), (3:7:4), (3:7:5), (4:7:2), (4:7:3), (4:7:4), (4:7:5), (5:7:2), (5:7:3), (5:7:4), (5:7:5), (2:6:2), (2:6:3), (2:6:4), (2:6:5), (3:6:2), (3:6:3), (3:6:4), (3:6:5), (4:6:2), (4:6:3), (4:6:4), (4:6:5), (5:6:2), (5:6:3), (5:6:4), or (5:6:5).

In some embodiments, the 5′-wing region (A′) comprises one or more locked nucleosides or 2′-substituted nucleosides. In some embodiments, the 3′-wing region (C′) comprises one or more locked nucleosides or 2′-substituted nucleosides. In some embodiments, the central region comprises one or more locked nucleosides or 2′-substituted nucleosides. The locked nucleoside can contain a bridge between the 4′ and the 2′ position of the sugar wherein the bridges comprises 2 to 4 optionally substituted atoms. For example, a locked nucleic acid (LNA) is:

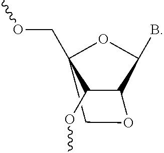

Other exemplary locked nucleosides include the following:

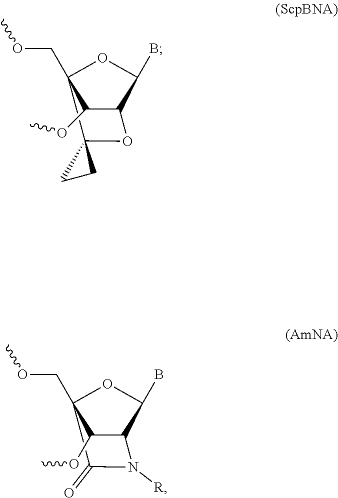

where R is H or alkyl (or AmNA(N-Me)) when R is alkyl);

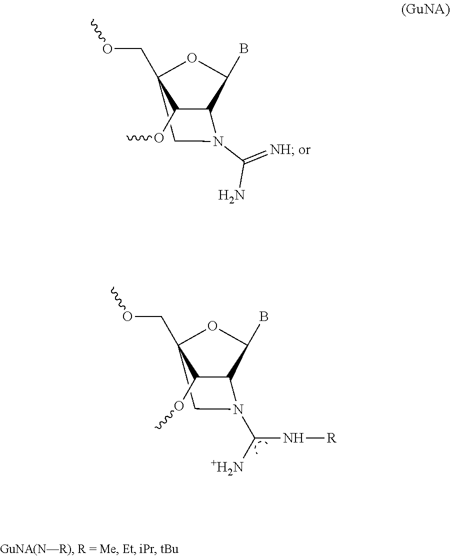

In some embodiments, all nucleosides in the 5′-wing region (A′) are locked nucleosides. In some embodiments, all nucleosides in the 3′-wing region (C′) are locked nucleosides. In some embodiments, the 3′-wing region comprises LNA and one or two nucleosides selected from ScpBNA, AmNA, and GuNA. In some embodiments, 5′-wing region are all LNA and the 3′-wing region contains LNA and one or two nucleosides selected from ScpBNA, AmNA, and GuNA. Other suitable nucleotides are included in PCT/JP2010/068409, PCT/JP2013/075370, PCT/JP2015/054308, PCT/JP2018/006061, and/or PCT/JP2018/006062, which are incorporated by reference in their entirety.

In some embodiments, one or more of the nucleotides in the 5′-wing region (A′) and/or the 3′-wing region (C′) comprises a thiophosphate internucleotide linkage. In certain embodiments, all nucleotides in the 5′-wing region (A′) comprises a thiophosphate internucleotide linkage. In some embodiments, the 5′-wing region of an ASO comprises 2 to 6 phosphorothioate-linked locked nucleosides. In some embodiments, the 5′-wing region comprises 2 to 6 phosphorothioate-linked 2′ substituted nucleosides. In some embodiments, the 5′-wing region comprises at least one locked nucleoside and at least one 2′ substituted nucleoside, wherein the locked nucleoside and the 2′ substituted nucleoside are linked by a phosphorothiate linker. In some embodiments, the 5′-wing region further comprises a RNA nucleoside or DNA nucleoside, wherein the RNA nucleoside and DNA nucleoside are not locked nucleosides or 2′-substituted nucleosides. In some embodiments, at least two nucleosides of the 5′-wing region are linked by a phosphorothioate linker. In some embodiments, at least 2, 3, 4, 5, or 6 nucleosides of the 5′-wing region are linked by a phosphorothioate linker.

In some embodiments, all nucleotides in the 3′-wing region (C′) comprise a thiophosphate internucleotide linkage. In some embodiments, the 3′-wing region of an ASO comprises 2 to 6 phosphorothioate-linked locked nucleosides. In some embodiments, the 3′-wing region comprises 2 to 6 2′ phosphorothioate-linked substituted nucleosides. In some embodiments, the 3′-wing region comprises at least one locked nucleoside and at least one 2′ substituted nucleoside, wherein the locked nucleoside and the 2′ substituted nucleoside are linked by a phosphorothiate linker. In some embodiments, the 3′-wing region further comprises a RNA nucleoside or DNA nucleoside, wherein the RNA nucleoside and DNA nucleoside are not locked nucleosides or 2′-substituted nucleosides. In some embodiments, at least two nucleosides of the 3′-wing region are linked by a phosphorothioate linker. In some embodiments, at least 2, 3, 4, 5, or 6 nucleosides of the 3′-wing region are linked by a phosphorothioate linker.

Suitable 2′-substituted nucleosides include, but are not limited to, 2′-O-methoxy nucleotides (e.g., mA, mU, mG, mC, etc.), 2′-O-methoxyethylribose nucleosides (e.g., moeA, moeT, moeG, etc.), and 5-methyl (5m) nucleotides (e.g., (5m)C, moe(5m)C, etc.). The disclosed ASOs may comprise 1, 2, 3, 4, 5, 6, 7, 8, 9, 10, 11, 12, 13, 14, 15, 16, 17, 18, 19, or 20 2′-substituted nucleosides, for example, the ASO may comprise 1 to 10, 2 to 10, 5 to 10, 6 to 10, 1 to 12, 2 to 12, 5 to 12, 6 to 12, 1 to 15, 5 to 15, or 10 to 15 2′-substituted nucleosides (e.g., 2′-O-methoxy nucleotides and/or 2′-O-methoxyethylribose nucleosides).

In some embodiments, one or more of the nucleotides in the 5′-wing region and/or the 3′-wing region comprises a thiophosphate internucleotide linkage. In some embodiments, all nucleotides in the 5′-wing region comprises a thiophosphate internucleotide linkage. In some embodiments, all nucleotides in the 3′-wing region comprises a thiophosphate internucleotide linkage.

In some embodiments, the central region includes one or more modified nucleotide having a modified nucleobase. For example, the central region can include one or more modified nucleotide having a protected or unprotected version of the following:

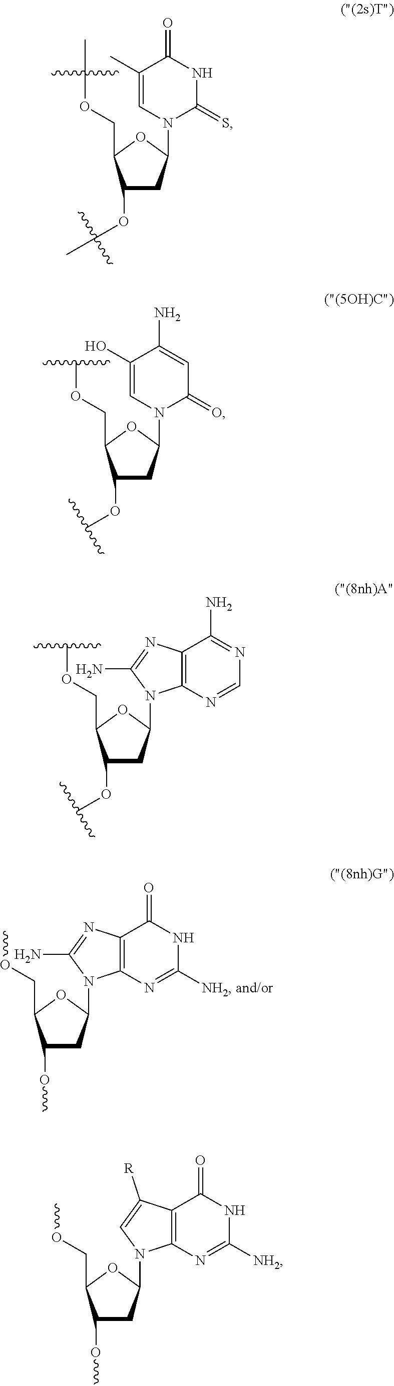

where R is a halogen or R′—C≡C—; and R′ is C₆₋₁₂ aryl, 5- to 12-membered heteroaryl, hydroxy-C₁₋₆ alkyl, or C₁₋₇ alkanoyloxy. In some embodiments, the central region includes one modified nucleotide (e.g., (2s)T or (5OH)C) at the 1^{st}, 2^{nd}, 3^{rd} or 4^{th} gap nucleoside position (from the 5′ end). In some embodiments, the modified nucleotide is at the 3^{rd} gap nucleoside position (from the 5′ end). In some embodiments, the modified nucleotide is a nucleotide having the structure of:

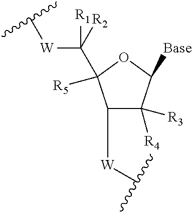

wherein:
W is independently O, N, or S;
R₁, R₂, and R₅ are independently H or D or CH₃ or CD₃;

### R₃ is H or F;

R₄ is F or OCH₃ or OCD₃; and

### Base is

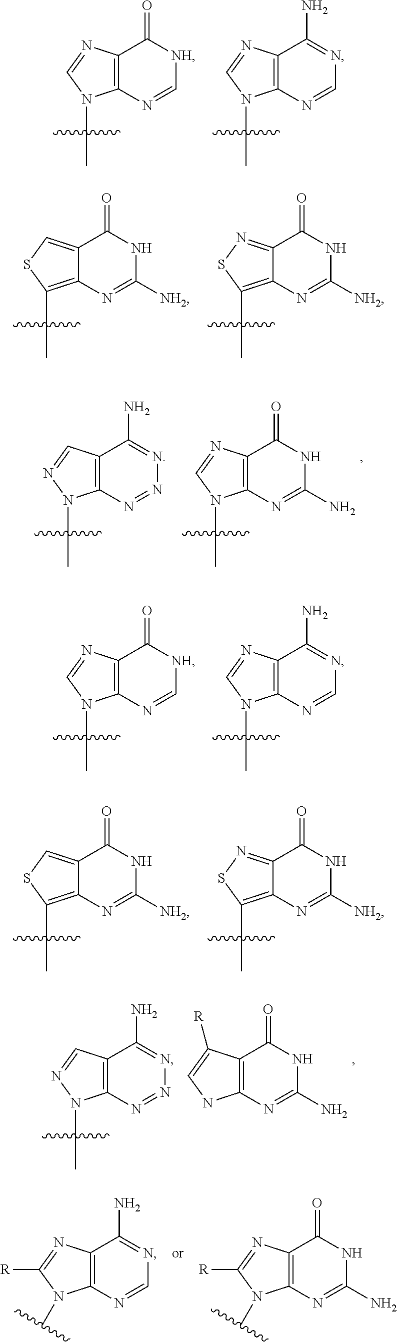

wherein:
R is a halogen or R′—C≡C—; and
R′ represents C₆₋₁₂ aryl, 5- to 12-membered heteroaryl, hydroxy-C₁₋₆ alkyl, or C₁₋₇ alkanoyloxy.
In some embodiments, C₁₋₇ alkanoyl includes, but is not limited to. formyl, acetyl, ethyl carbonyl, n-propyl carbonyl, isopropyl carbonyl, n-butyl carbonyl, isobutyl carbonyl, t-butyl carbonyl, n-pentyl carbonyl, and n-hexyl carbonyl. Other modified nucleotides include those in PCT/JP2018/006061, which is incorporated by reference in its entirety.

As used herein, unless otherwise indicated, “aryl” refers to a carbocyclic (all carbon) ring that has a fully delocalized pi-electron system. The “aryl” group can be made up of two or more fused rings (rings that share two adjacent carbon atoms). When the aryl is a fused ring system, then the ring that is connected to the rest of the molecule has a fully delocalized pi-electron system. The other ring(s) in the fused ring system may or may not have a fully delocalized pi-electron system. Examples of aryl groups include, without limitation, the radicals of benzene, naphthalene and azulene.

As used herein, unless otherwise indicated, “heteroaryl” refers to a ring that has a fully delocalized pi-electron system and contains one or more heteroatoms (e.g., one to three heteroatoms, or one to four heteroatoms, or one to five heteroatoms) independently selected from the group consisting of nitrogen, oxygen, and sulfur in the ring. The “heteroaryl” group can be made up of two or more fused rings (rings that share two adjacent carbon atoms). When the heteroaryl is a fused ring system, then the ring that is connected to the rest of the molecule has a fully delocalized pi-electron system. The other ring(s) in the fused ring system may or may not have a fully delocalized pi-electron system. Examples of heteroaryl rings include, without limitation, furan, thiophene, pyrrole, oxazole, thiazole, imidazole, pyrazole, isoxazole, isothiazole, triazole, thiadiazole, pyridine, pyridazine, pyrimidine, pyrazine and triazine.

In some embodiments, the central region of an ASO comprises at least 5 contiguous phosphorothioate-linked DNA nucleosides. In some embodiments, at least 2, 3, 4, 5, or 6 nucleosides of the central region are linked by a phosphorothioate linker. In some embodiments, a DNA nucleoside of central region is linked to a nucleoside of a 5′-wing region by a phosphorothioate linker. In some embodiments, a DNA nucleoside of central region is linked to a nucleoside of a 3′-wing region by a phosphorothioate linker. In some embodiments, the central region comprises 8 to 10 contiguous phosphorothioate-linked DNA nucleosides.

In some embodiments, the ASO is complementary or hybridizes to a viral target RNA sequence (i.e., a sequence of a SARS-CoV-2 genome) that comprises, consists of, or consists essentially of at least 5, 6, 7, 8, 9, 10, 11, 12, 13, 14, or 15 contiguous nucleotides within any one of SEQ ID NOs: 5535-5538. In some embodiments, the ASO is complementary or hybridizes to a viral target RNA sequence that comprises, consists of, or consists essentially of 5 to 15, 5 to 14, 5 to 13, 5 to 12, 5 to 11, 5 to 10, 5 to 9, 5 to 8, 6 to 15, 6 to 14, 6 to 13, 6 to 12, 6 to 11, 6 to 10, 7 to 15, 7 to 14, 7 to 13, 7 to 12, or 7 to 11 contiguous nucleotides within any one of SEQ ID NOs: 5535-5538. In some embodiments, the ASO is perfectly complementary to the viral target RNA sequence. In some embodiments, there is less than or equal to 5, 4, 3, 2, or 1 mismatches between the ASO and the viral target sequence. In some embodiments, there is less than or equal to 2 mismatches between the ASO and the viral target sequence. In some embodiments, there is less than or equal to 1 mismatch between the ASO and the viral target sequence. In some embodiments, the mismatch is in the wing region of the ASO. In some embodiments, the mismatch is in the 5′ wing region of the ASO. In some embodiments, the mismatch is in the 3′ wing region of the ASO. In some embodiments, the mismatch is in the central region of the ASO.

In some embodiments, the central region is complementary or hybridizes to a viral target RNA sequence that comprises, consists of, or consists essentially of at least 5, 6, 7, 8, 9, 10, 11, 12, 13, 14, or 15 contiguous nucleotides within any one of SEQ ID NOs: 5535-5538. In some embodiments, the central region is complementary or hybridizes to a viral target RNA sequence that comprises, consists of, or consists essentially of 5 to 15, 5 to 14, 5 to 13, 5 to 12, 5 to 11, 5 to 10, 5 to 9, 5 to 8, 6 to 15, 6 to 14, 6 to 13, 6 to 12, 6 to 11, 6 to 10, 7 to 15, 7 to 14, 7 to 13, 7 to 12, or 7 to 11 contiguous nucleotides within any one of SEQ ID NOs: 5535-5538. In some embodiments, the central region is perfectly complementary to the viral target RNA sequence. In some embodiments, there is less than or equal to 5, 4, 3, 2, or 1 mismatches between the central region and the viral target sequence. In some embodiments, there is less than or equal to 2 mismatches between the central region and the viral target sequence. In some embodiments, there is less than or equal to 1 mismatch between the central region and the viral target sequence.

The coronavidae family of viruses comprises four genera and the genome of these viruses encodes 16 non-structural proteins and 12 structural or accessory proteins, for a total of 28 total proteins across multiple different open reading frames (see, e.g., FIG. 1). In some embodiments, the ASO is complementary or hybridizes to a viral target RNA sequence is complementary or hybridizes to a viral target RNA sequence that begins in a region encoding a non-structural protein (nsp), such as leader protein, nsp2, nsp3, nsp4, nsp5, nsp6, nsp7, nsp8, nsp9, nsp10, nsp11, nsp12, nsp13, nsp14, nsp15, or nsp16. In some embodiments, the viral target RNA sequence may preferably begin in a region encoding nsp8, nsp9, nsp10, nsp11, nsp12, nsp13, nsp14, or nsp15. In some embodiments, the ASO is complementary or hybridizes to a viral target RNA sequence that begins in a region encoding a structural or accessory protein, such as an S region, an E region, an M region, or an N region of a coronavirus. In some embodiments, the viral target is a region of the coronavirus genome that share substantial homology across multiple members of the coronavirus family (see, e.g., FIG. 2). Table 2 at the end of the Detailed Description Section provides exemplary coronavirus genomes from which homologous target regions can be established.

In some embodiments, the viral target may, e.g., begin at the 5′-end of target-site in GenBank Accession NC_045512.2, or in any other region of homology shared between the coronavirus genomes disclosed in Table 2 In some embodiments, the ASO directed to a coronavirus target region may comprise 8, 9, 10, 11, 12, 13, 14, 15, or 16 nucleotides/nucleosides of any one of the sequences disclosed in Table 1 or a complement thereof. Any of the nucleotide/nucleosides in an ASO comprising 8, 9, 10, 11, 12, 13, 14, 15, or 16 nucleotides/nucleosides of a sequence disclosed in Table 1 or a complement thereof may be modified, locked, conjugated or linked, as further described herein. In other words, any of the sequences disclosed in Table 1 may, in some embodiments, comprise one or more modified, locked, conjugated or linked nucleotides/nucleosides in addition to or in place of the nucleotides/nucleosides disclosed in SEQ ID NOs: 1-5534. For example, any one of SEQ ID NOs: 1-5534 may comprise one or more locked nucleic acid (LNA) A; (5m)lnC=locked nucleic acid (LNA)-5methyl C; lnG=locked nucleic acid (LNA) G; lnT=locked nucleic acid (LNA) T; (5m)C=5 methylC; mA=2-O-methoxy A; mU=2-O-methoxy U; (8nh)A=8-amino A; (8nh)G=8-amino G; (2s)T=2-thio T; (5-OH)C=5-hydroxy C; cp=scp=cyclopropyl; cp (5m)C=scp (5m) C=cyclopropyl (5m) C; cpG=scpG=cyclopropyl G; cpT=scpT=cyclopropyl T; or ps=phosphorothioate linkages.

TABLE 1
Exemplary ASO Sequences

| SEQ | | | | | |
|---|---|---|---|---|---|
| ID | | | Polarity to | Start on | End on |
| NO: | Oligo sequence | Target | target | target | target |
| 1 | GTCAACGACAGTAATT | NC_045512.2 | 3′→5′ | 142 | 157 |
| 2 | TGTCAACGACAGTAAT | NC_045512.2 | 3′→5′ | 143 | 158 |
| 3 | ACGAAACCGTAAGCAG | NC_045512.2 | 3′→5′ | 190 | 205 |
| 4 | GACGAAACCGTAAGCA | NC_045512.2 | 3′→5′ | 191 | 206 |
| 5 | GGACGAAACCGTAAGC | NC_045512.2 | 3′→5′ | 192 | 207 |
| 6 | CGGACGAAACCGTAAG | NC_045512.2 | 3′→5′ | 193 | 208 |
| 7 | ACGGACGAAACCGTAA | NC_045512.2 | 3′→5′ | 194 | 209 |
| 8 | CACGGACGAAACCGTA | NC_045512.2 | 3′→5′ | 195 | 210 |
| 9 | ACACGGACGAAACCGT | NC_045512.2 | 3′→5′ | 196 | 211 |
| 10 | AACACGGACGAAACCG | NC_045512.2 | 3′→5′ | 197 | 212 |
| 11 | CAACACGGACGAAACC | NC_045512.2 | 3′→5′ | 198 | 213 |
| 12 | GCAACACGGACGAAAC | NC_045512.2 | 3′→5′ | 199 | 214 |
| 13 | TGCAACACGGACGAAA | NC_045512.2 | 3′→5′ | 200 | 215 |
| 14 | CTGCAACACGGACGAA | NC_045512.2 | 3′→5′ | 201 | 216 |
| 15 | CCCGGACGAAACCTAG | NC_045512.2 | 3′→5′ | 233 | 248 |
| 16 | ACCCGGACGAAACCTA | NC_045512.2 | 3′→5′ | 234 | 249 |
| 17 | CACCCGGACGAAACCT | NC_045512.2 | 3′→5′ | 235 | 250 |
| 18 | ACACCCGGACGAAACC | NC_045512.2 | 3′→5′ | 236 | 251 |
| 19 | CACACCCGGACGAAAC | NC_045512.2 | 3′→5′ | 237 | 252 |
| 20 | TCACACCCGGACGAAA | NC_045512.2 | 3′→5′ | 238 | 253 |
| 21 | GTCACACCCGGACGAA | NC_045512.2 | 3′→5′ | 239 | 254 |
| 22 | GGTCACACCCGGACGA | NC_045512.2 | 3′→5′ | 240 | 255 |
| 23 | CGGTCACACCCGGACG | NC_045512.2 | 3′→5′ | 241 | 256 |
| 24 | TCGGTCACACCCGGAC | NC_045512.2 | 3′→5′ | 242 | 257 |
| 25 | TTCGGTCACACCCGGA | NC_045512.2 | 3′→5′ | 243 | 258 |
| 26 | TTTCGGTCACACCCGG | NC_045512.2 | 3′→5′ | 244 | 259 |
| 27 | CTTTCGGTCACACCCG | NC_045512.2 | 3′→5′ | 245 | 260 |
| 28 | CCTTTCGGTCACACCC | NC_045512.2 | 3′→5′ | 246 | 261 |
| 29 | ACCTTTCGGTCACACC | NC_045512.2 | 3′→5′ | 247 | 262 |
| 30 | TACCTTTCGGTCACAC | NC_045512.2 | 3′→5′ | 248 | 263 |
| 31 | TTACCTTTCGGTCACA | NC_045512.2 | 3′→5′ | 249 | 264 |
| 32 | CTTACCTTTCGGTCAC | NC_045512.2 | 3′→5′ | 250 | 265 |
| 33 | TCTTACCTTTCGGTCA | NC_045512.2 | 3′→5′ | 251 | 266 |
| 34 | ATCTTACCTTTCGGTC | NC_045512.2 | 3′→5′ | 252 | 267 |
| 35 | CATCTTACCTTTCGGT | NC_045512.2 | 3′→5′ | 253 | 268 |
| 36 | CCATCTTACCTTTCGG | NC_045512.2 | 3′→5′ | 254 | 269 |
| 37 | TCCATCTTACCTTTCG | NC_045512.2 | 3′→5′ | 255 | 270 |
| 38 | CTCCATCTTACCTTTC | NC_045512.2 | 3′→5′ | 256 | 271 |
| 39 | TCTCCATCTTACCTTT | NC_045512.2 | 3′→5′ | 257 | 272 |
| 40 | CTCTCCATCTTACCTT | NC_045512.2 | 3′→5′ | 258 | 273 |
| 41 | GCTCTCCATCTTACCT | NC_045512.2 | 3′→5′ | 259 | 274 |
| 42 | GGCTCTCCATCTTACC | NC_045512.2 | 3′→5′ | 260 | 275 |
| 43 | AGGCTCTCCATCTTAC | NC_045512.2 | 3′→5′ | 261 | 276 |
| 44 | AAGGCTCTCCATCTTA | NC_045512.2 | 3′→5′ | 262 | 277 |
| 45 | CAAGGCTCTCCATCTT | NC_045512.2 | 3′→5′ | 263 | 278 |
| 46 | ACAAGGCTCTCCATCT | NC_045512.2 | 3′→5′ | 264 | 279 |
| 47 | TGTGTTTTCTCGTTGA | NC_045512.2 | 3′→5′ | 288 | 303 |
| 48 | GTGTGTTTTCTCGTTG | NC_045512.2 | 3′→5′ | 289 | 304 |
| 49 | CGTGTGTTTTCTCGTT | NC_045512.2 | 3′→5′ | 290 | 305 |
| 50 | ACGTGTGTTTTCTCGT | NC_045512.2 | 3′→5′ | 291 | 306 |
| 51 | GACGTGTGTTTTCTCG | NC_045512.2 | 3′→5′ | 292 | 307 |
| 52 | GGACGTGTGTTTTCTC | NC_045512.2 | 3′→5′ | 293 | 308 |
| 53 | TGGACGTGTGTTTTCT | NC_045512.2 | 3′→5′ | 294 | 309 |
| 54 | TTGGACGTGTGTTTTC | NC_045512.2 | 3′→5′ | 295 | 310 |
| 55 | GTTGGACGTGTGTTTT | NC_045512.2 | 3′→5′ | 296 | 311 |
| 56 | AGTTGGACGTGTGTTT | NC_045512.2 | 3′→5′ | 297 | 312 |
| 57 | GAGTTGGACGTGTGTT | NC_045512.2 | 3′→5′ | 298 | 313 |
| 58 | TGAGTTGGACGTGTGT | NC_045512.2 | 3′→5′ | 299 | 314 |
| 59 | CTGAGTTGGACGTGTG | NC_045512.2 | 3′→5′ | 300 | 315 |
| 60 | ACTGAGTTGGACGTGT | NC_045512.2 | 3′→5′ | 301 | 316 |
| 61 | AACTGAGTTGGACGTG | NC_045512.2 | 3′→5′ | 302 | 317 |
| 62 | AAACTGAGTTGGACGT | NC_045512.2 | 3′→5′ | 303 | 318 |
| 63 | CAAACTGAGTTGGACG | NC_045512.2 | 3′→5′ | 304 | 319 |
| 64 | GCAAACTGAGTTGGAC | NC_045512.2 | 3′→5′ | 305 | 320 |
| 65 | GGCAAACTGAGTTGGA | NC_045512.2 | 3′→5′ | 306 | 321 |
| 66 | AGGCAAACTGAGTTGG | NC_045512.2 | 3′→5′ | 307 | 322 |
| 67 | CAGGCAAACTGAGTTG | NC_045512.2 | 3′→5′ | 308 | 323 |
| 68 | ACAGGCAAACTGAGTT | NC_045512.2 | 3′→5′ | 309 | 324 |
| 69 | CATAGGGCTGTTCAAG | NC_045512.2 | 3′→5′ | 455 | 470 |
| 70 | ACATAGGGCTGTTCAA | NC_045512.2 | 3′→5′ | 456 | 471 |
| 71 | CACATAGGGCTGTTCA | NC_045512.2 | 3′→5′ | 457 | 472 |
| 72 | ACACATAGGGCTGTTC | NC_045512.2 | 3′→5′ | 458 | 473 |
| 73 | AACACATAGGGCTGTT | NC_045512.2 | 3′→5′ | 459 | 474 |
| 74 | GAACACATAGGGCTGT | NC_045512.2 | 3′→5′ | 460 | 475 |
| 75 | TGAACACATAGGGCTG | NC_045512.2 | 3′→5′ | 461 | 476 |
| 76 | ATGAACACATAGGGCT | NC_045512.2 | 3′→5′ | 462 | 477 |
| 77 | TCTTACGAAGAAGAAC | NC_045512.2 | 3′→5′ | 626 | 641 |
| 78 | TTCTTACGAAGAAGAA | NC_045512.2 | 3′→5′ | 627 | 642 |
| 79 | GTTCTTACGAAGAAGA | NC_045512.2 | 3′→5′ | 628 | 643 |
| 80 | CGTTCTTACGAAGAAG | NC_045512.2 | 3′→5′ | 629 | 644 |
| 81 | CCGTTCTTACGAAGAA | NC_045512.2 | 3′→5′ | 630 | 645 |
| 82 | ACCGTTCTTACGAAGA | NC_045512.2 | 3′→5′ | 631 | 646 |
| 83 | TACCGTTCTTACGAAG | NC_045512.2 | 3′→5′ | 632 | 647 |
| 84 | TTACCGTTCTTACGAA | NC_045512.2 | 3′→5′ | 633 | 648 |
| 85 | ATTACCGTTCTTACGA | NC_045512.2 | 3′→5′ | 634 | 649 |
| 86 | TATTACCGTTCTTACG | NC_045512.2 | 3′→5′ | 635 | 650 |
| 87 | TTATTACCGTTCTTAC | NC_045512.2 | 3′→5′ | 636 | 651 |
| 88 | CAGTGCCAAGCTCGTC | NC_045512.2 | 3′→5′ | 704 | 719 |
| 89 | TCAGTGCCAAGCTCGT | NC_045512.2 | 3′→5′ | 705 | 720 |
| 90 | ATCAGTGCCAAGCTCG | NC_045512.2 | 3′→5′ | 706 | 721 |
| 91 | GATCAGTGCCAAGCTC | NC_045512.2 | 3′→5′ | 707 | 722 |
| 92 | GGATCAGTGCCAAGCT | NC_045512.2 | 3′→5′ | 708 | 723 |
| 93 | TTAGTGTTCCAGTTTT | NC_045512.2 | 3′→5′ | 741 | 756 |
| 94 | ACACACAGCCTCCAAA | NC_045512.2 | 3′→5′ | 1481 | 1496 |
| 95 | AACACACAGCCTCCAA | NC_045512.2 | 3′→5′ | 1482 | 1497 |
| 96 | AAGTTTTAAATAACCA | NC_045512.2 | 3′→5′ | 3352 | 3367 |
| 97 | TAAGTTTTAAATAACC | NC_045512.2 | 3′→5′ | 3353 | 3368 |
| 98 | GTAAGTTTTAAATAAC | NC_045512.2 | 3′→5′ | 3354 | 3369 |
| 99 | AGTAAGTTTTAAATAA | NC_045512.2 | 3′→5′ | 3355 | 3370 |
| 100 | CAGTAAGTTTTAAATA | NC_045512.2 | 3′→5′ | 3356 | 3371 |
| 101 | TCAGTAAGTTTTAAAT | NC_045512.2 | 3′→5′ | 3357 | 3372 |
| 102 | GTCAGTAAGTTTTAAA | NC_045512.2 | 3′→5′ | 3358 | 3373 |
| 103 | TGTCAGTAAGTTTTAA | NC_045512.2 | 3′→5′ | 3359 | 3374 |
| 104 | TTGTCAGTAAGTTTTA | NC_045512.2 | 3′→5′ | 3360 | 3375 |
| 105 | ATTGTCAGTAAGTTTT | NC_045512.2 | 3′→5′ | 3361 | 3376 |
| 106 | CATTGTCAGTAAGTTT | NC_045512.2 | 3′→5′ | 3362 | 3377 |
| 107 | ACATTGTCAGTAAGTT | NC_045512.2 | 3′→5′ | 3363 | 3378 |
| 108 | ACTTAGTTTCTTCCAG | NC_045512.2 | 3′→5′ | 3992 | 4007 |
| 109 | AACTTAGTTTCTTCCA | NC_045512.2 | 3′→5′ | 3993 | 4008 |
| 110 | CACAAAAGTTAGCAGC | NC_045512.2 | 3′→5′ | 5384 | 5399 |
| 111 | GCACAAAAGTTAGCAG | NC_045512.2 | 3′→5′ | 5385 | 5400 |
| 112 | TGCACAAAAGTTAGCA | NC_045512.2 | 3′→5′ | 5386 | 5401 |
| 113 | GTGCACAAAAGTTAGC | NC_045512.2 | 3′→5′ | 5387 | 5402 |
| 114 | AGTGCACAAAAGTTAG | NC_045512.2 | 3′→5′ | 5388 | 5403 |
| 115 | GACATCATAACAAAAG | NC_045512.2 | 3′→5′ | 5673 | 5688 |
| 116 | CTCCAAAGACAACGTA | NC_045512.2 | 3′→5′ | 6294 | 6309 |
| 117 | CACTACTTCTTCAGAG | NC_045512.2 | 3′→5′ | 6406 | 6421 |
| 118 | CCACTACTTCTTCAGA | NC_045512.2 | 3′→5′ | 6407 | 6422 |
| 119 | TCCACTACTTCTTCAG | NC_045512.2 | 3′→5′ | 6408 | 6423 |
| 120 | TTCCACTACTTCTTCA | NC_045512.2 | 3′→5′ | 6409 | 6424 |
| 121 | TTTCCACTACTTCTTC | NC_045512.2 | 3′→5′ | 6410 | 6425 |
| 122 | TTTTCCACTACTTCTT | NC_045512.2 | 3′→5′ | 6411 | 6426 |
| 123 | ATTTTCCACTACTTCT | NC_045512.2 | 3′→5′ | 6412 | 6427 |
| 124 | GATTTTCCACTACTTC | NC_045512.2 | 3′→5′ | 6413 | 6428 |
| 125 | GGATTTTCCACTACTT | NC_045512.2 | 3′→5′ | 6414 | 6429 |
| 126 | AGGATTTTCCACTACT | NC_045512.2 | 3′→5′ | 6415 | 6430 |
| 127 | TAGGATTTTCCACTAC | NC_045512.2 | 3′→5′ | 6416 | 6431 |
| 128 | GTAGGATTTTCCACTA | NC_045512.2 | 3′→5′ | 6417 | 6432 |
| 129 | GGTAGGATTTTCCACT | NC_045512.2 | 3′→5′ | 6418 | 6433 |
| 130 | CTTCGGTAGTTTTCAC | NC_045512.2 | 3′→5′ | 6461 | 6476 |
| 131 | ACTTCGGTAGTTTTCA | NC_045512.2 | 3′→5′ | 6462 | 6477 |
| 132 | AACTTCGGTAGTTTTC | NC_045512.2 | 3′→5′ | 6463 | 6478 |
| 133 | CAACTTCGGTAGTTTT | NC_045512.2 | 3′→5′ | 6464 | 6479 |
| 134 | ACAACTTCGGTAGTTT | NC_045512.2 | 3′→5′ | 6465 | 6480 |
| 135 | TACAACTTCGGTAGTT | NC_045512.2 | 3′→5′ | 6466 | 6481 |
| 136 | CTACAACTTCGGTAGT | NC_045512.2 | 3′→5′ | 6467 | 6482 |
| 137 | CCTACAACTTCGGTAG | NC_045512.2 | 3′→5′ | 6468 | 6483 |
| 138 | CATTAGGTTTCTTAAT | NC_045512.2 | 3′→5′ | 6584 | 6599 |
| 139 | TCATTAGGTTTCTTAA | NC_045512.2 | 3′→5′ | 6585 | 6600 |
| 140 | TAACAATAGTTGTACA | NC_045512.2 | 3′→5′ | 7532 | 7547 |
| 141 | TTAACAATAGTTGTAC | NC_045512.2 | 3′→5′ | 7533 | 7548 |
| 142 | ATTAACAATAGTTGTA | NC_045512.2 | 3′→5′ | 7534 | 7549 |
| 143 | CATTAACAATAGTTGT | NC_045512.2 | 3′→5′ | 7535 | 7550 |
| 144 | CCATTAACAATAGTTG | NC_045512.2 | 3′→5′ | 7536 | 7551 |
| 145 | TTGGTCTTTTAAACTG | NC_045512.2 | 3′→5′ | 7691 | 7706 |
| 146 | ATTGGTCTTTTAAACT | NC_045512.2 | 3′→5′ | 7692 | 7707 |
| 147 | TGTGACTTTTTGCTAC | NC_045512.2 | 3′→5′ | 8372 | 8387 |
| 148 | TTGTGACTTTTTGCTA | NC_045512.2 | 3′→5′ | 8373 | 8388 |
| 149 | GTCACGAGTGACACCA | NC_045512.2 | 3′→5′ | 8707 | 8722 |
| 150 | TGTCACGAGTGACACC | NC_045512.2 | 3′→5′ | 8708 | 8723 |
| 151 | ATGTCACGAGTGACAC | NC_045512.2 | 3′→5′ | 8709 | 8724 |
| 152 | GTCAAGTACAAGTAAA | NC_045512.2 | 3′→5′ | 9588 | 9603 |
| 153 | TGTCAAGTACAAGTAA | NC_045512.2 | 3′→5′ | 9589 | 9604 |
| 154 | ATGTCAAGTACAAGTA | NC_045512.2 | 3′→5′ | 9590 | 9605 |
| 155 | AATGTCAAGTACAAGT | NC_045512.2 | 3′→5′ | 9591 | 9606 |
| 156 | CTAAAACCACTCTGCA | NC_045512.2 | 3′→5′ | 10050 | 10065 |
| 157 | CATGTCTTGGACAGTA | NC_045512.2 | 3′→5′ | 10163 | 10178 |
| 158 | ACATGTCTTGGACAGT | NC_045512.2 | 3′→5′ | 10164 | 10179 |
| 159 | ATGGTGAACCATTGTA | NC_045512.2 | 3′→5′ | 10406 | 10421 |
| 160 | GATGGTGAACCATTGT | NC_045512.2 | 3′→5′ | 10407 | 10422 |
| 161 | CAACACTACCACATGA | NC_045512.2 | 3′→5′ | 10484 | 10499 |
| 162 | CCAACACTACCACATG | NC_045512.2 | 3′→5′ | 10485 | 10500 |
| 163 | ACCAACACTACCACAT | NC_045512.2 | 3′→5′ | 10486 | 10501 |
| 164 | AACCAACACTACCACA | NC_045512.2 | 3′→5′ | 10487 | 10502 |
| 165 | AAACCAACACTACCAC | NC_045512.2 | 3′→5′ | 10488 | 10503 |
| 166 | AAAACCAACACTACCA | NC_045512.2 | 3′→5′ | 10489 | 10504 |
| 167 | TAAAACCAACACTACC | NC_045512.2 | 3′→5′ | 10490 | 10505 |
| 168 | TTAAAACCAACACTAC | NC_045512.2 | 3′→5′ | 10491 | 10506 |
| 169 | GTTAAAACCAACACTA | NC_045512.2 | 3′→5′ | 10492 | 10507 |
| 170 | TGTTAAAACCAACACT | NC_045512.2 | 3′→5′ | 10493 | 10508 |
| 171 | ATGTTAAAACCAACAC | NC_045512.2 | 3′→5′ | 10494 | 10509 |
| 172 | GCAAGAGAAGGTAACA | NC_045512.2 | 3′→5′ | 11184 | 11199 |
| 173 | CTAAGAAACAATAAAC | NC_045512.2 | 3′→5′ | 11609 | 11624 |
| 174 | CCTAAGAAACAATAAA | NC_045512.2 | 3′→5′ | 11610 | 11625 |
| 175 | GCCTAAGAAACAATAA | NC_045512.2 | 3′→5′ | 11611 | 11626 |
| 176 | AGCCTAAGAAACAATA | NC_045512.2 | 3′→5′ | 11612 | 11627 |
| 177 | TAGCCTAAGAAACAAT | NC_045512.2 | 3′→5′ | 11613 | 11628 |
| 178 | ATAGCCTAAGAAACAA | NC_045512.2 | 3′→5′ | 11614 | 11629 |
| 179 | AATAGCCTAAGAAACA | NC_045512.2 | 3′→5′ | 11615 | 11630 |
| 180 | CATAAACACCAAGAGT | NC_045512.2 | 3′→5′ | 11684 | 11699 |
| 181 | TCATAAACACCAAGAG | NC_045512.2 | 3′→5′ | 11685 | 11700 |
| 182 | TTTTAGACTGTACAGT | NC_045512.2 | 3′→5′ | 11834 | 11849 |
| 183 | ATTTTAGACTGTACAG | NC_045512.2 | 3′→5′ | 11835 | 11850 |
| 184 | CATTTTAGACTGTACA | NC_045512.2 | 3′→5′ | 11836 | 11851 |
| 185 | ACATTTTAGACTGTAC | NC_045512.2 | 3′→5′ | 11837 | 11852 |
| 186 | GACATTTTAGACTGTA | NC_045512.2 | 3′→5′ | 11838 | 11853 |
| 187 | CAGCACCCTGCATGGA | NC_045512.2 | 3′→5′ | 12023 | 12038 |
| 188 | ACAGCACCCTGCATGG | NC_045512.2 | 3′→5′ | 12024 | 12039 |
| 189 | TACAGCACCCTGCATG | NC_045512.2 | 3′→5′ | 12025 | 12040 |
| 190 | CTACAGCACCCTGCAT | NC_045512.2 | 3′→5′ | 12026 | 12041 |
| 191 | TCTACAGCACCCTGCA | NC_045512.2 | 3′→5′ | 12027 | 12042 |
| 192 | GTCTACAGCACCCTGC | NC_045512.2 | 3′→5′ | 12028 | 12043 |
| 193 | TGTCTACAGCACCCTG | NC_045512.2 | 3′→5′ | 12029 | 12044 |
| 194 | ATGTCTACAGCACCCT | NC_045512.2 | 3′→5′ | 12030 | 12045 |
| 195 | CTTCAGAATCACCATT | NC_045512.2 | 3′→5′ | 12173 | 12188 |
| 196 | ACTTCAGAATCACCAT | NC_045512.2 | 3′→5′ | 12174 | 12189 |
| 197 | TAGCCACATTCAAAGA | NC_045512.2 | 3′→5′ | 12212 | 12227 |
| 198 | TTAGCCACATTCAAAG | NC_045512.2 | 3′→5′ | 12213 | 12228 |
| 199 | TTTAGCCACATTCAAA | NC_045512.2 | 3′→5′ | 12214 | 12229 |
| 200 | ATTTAGCCACATTCAA | NC_045512.2 | 3′→5′ | 12215 | 12230 |
| 201 | GATTTAGCCACATTCA | NC_045512.2 | 3′→5′ | 12216 | 12231 |
| 202 | AGATTTAGCCACATTC | NC_045512.2 | 3′→5′ | 12217 | 12232 |
| 203 | CAGATTTAGCCACATT | NC_045512.2 | 3′→5′ | 12218 | 12233 |
| 204 | TCAGATTTAGCCACAT | NC_045512.2 | 3′→5′ | 12219 | 12234 |
| 205 | CCATCTTTTCCAACTT | NC_045512.2 | 3′→5′ | 12263 | 12278 |
| 206 | GCCATCTTTTCCAACT | NC_045512.2 | 3′→5′ | 12264 | 12279 |
| 207 | ACATTTGGGTCATAGC | NC_045512.2 | 3′→5′ | 12287 | 12302 |
| 208 | TACATTTGGGTCATAG | NC_045512.2 | 3′→5′ | 12288 | 12303 |
| 209 | CTTTTGCCCTCTTGTC | NC_045512.2 | 3′→5′ | 12323 | 12338 |
| 210 | ACTTTTGCCCTCTTGT | NC_045512.2 | 3′→5′ | 12324 | 12339 |
| 211 | TGTTGATAATGTTGTT | NC_045512.2 | 3′→5′ | 12401 | 12416 |
| 212 | TTGTTGATAATGTTGT | NC_045512.2 | 3′→5′ | 12402 | 12417 |
| 213 | ATTGTTGATAATGTTG | NC_045512.2 | 3′→5′ | 12403 | 12418 |
| 214 | CATTGTTGATAATGTT | NC_045512.2 | 3′→5′ | 12404 | 12419 |
| 215 | GCATTGTTGATAATGT | NC_045512.2 | 3′→5′ | 12405 | 12420 |
| 216 | ACATCTGTCGTAGTGC | NC_045512.2 | 3′→5′ | 12707 | 12722 |
| 217 | GACATCTGTCGTAGTG | NC_045512.2 | 3′→5′ | 12708 | 12723 |
| 218 | CAAACCTACCTCCCTT | NC_045512.2 | 3′→5′ | 12791 | 12806 |
| 219 | ACAAACCTACCTCCCT | NC_045512.2 | 3′→5′ | 12792 | 12807 |
| 220 | GGAATCTAGCCCATTT | NC_045512.2 | 3′→5′ | 12839 | 12854 |
| 221 | GGGAATCTAGCCCATT | NC_045512.2 | 3′→5′ | 12840 | 12855 |
| 222 | AGGGAATCTAGCCCAT | NC_045512.2 | 3′→5′ | 12841 | 12856 |
| 223 | TAGGGAATCTAGCCCA | NC_045512.2 | 3′→5′ | 12842 | 12857 |
| 224 | TTAGGGAATCTAGCCC | NC_045512.2 | 3′→5′ | 12843 | 12858 |
| 225 | CTTAGGGAATCTAGCC | NC_045512.2 | 3′→5′ | 12844 | 12859 |
| 226 | TCTTAGGGAATCTAGC | NC_045512.2 | 3′→5′ | 12845 | 12860 |
| 227 | CTCTTAGGGAATCTAG | NC_045512.2 | 3′→5′ | 12846 | 12861 |
| 228 | ACTCTTAGGGAATCTA | NC_045512.2 | 3′→5′ | 12847 | 12862 |
| 229 | CACTCTTAGGGAATCT | NC_045512.2 | 3′→5′ | 12848 | 12863 |
| 230 | TCACTCTTAGGGAATC | NC_045512.2 | 3′→5′ | 12849 | 12864 |
| 231 | ATCACTCTTAGGGAAT | NC_045512.2 | 3′→5′ | 12850 | 12865 |
| 232 | CATCACTCTTAGGGAA | NC_045512.2 | 3′→5′ | 12851 | 12866 |
| 233 | CCATCACTCTTAGGGA | NC_045512.2 | 3′→5′ | 12852 | 12867 |
| 234 | GGTGGTTCCAGTTCTG | NC_045512.2 | 3′→5′ | 12885 | 12900 |
| 235 | AGGTGGTTCCAGTTCT | NC_045512.2 | 3′→5′ | 12886 | 12901 |
| 236 | AAGGTGGTTCCAGTTC | NC_045512.2 | 3′→5′ | 12887 | 12902 |
| 237 | CAAGGTGGTTCCAGTT | NC_045512.2 | 3′→5′ | 12888 | 12903 |
| 238 | ACAAGGTGGTTCCAGT | NC_045512.2 | 3′→5′ | 12889 | 12904 |
| 239 | TACAAGGTGGTTCCAG | NC_045512.2 | 3′→5′ | 12890 | 12905 |
| 240 | CTACAAGGTGGTTCCA | NC_045512.2 | 3′→5′ | 12891 | 12906 |
| 241 | CCTACAAGGTGGTTCC | NC_045512.2 | 3′→5′ | 12892 | 12907 |
| 242 | ACCTACAAGGTGGTTC | NC_045512.2 | 3′→5′ | 12893 | 12908 |
| 243 | AACCTACAAGGTGGTT | NC_045512.2 | 3′→5′ | 12894 | 12909 |
| 244 | AAACCTACAAGGTGGT | NC_045512.2 | 3′→5′ | 12895 | 12910 |
| 245 | CAAACCTACAAGGTGG | NC_045512.2 | 3′→5′ | 12896 | 12911 |
| 246 | ACAAACCTACAAGGTG | NC_045512.2 | 3′→5′ | 12897 | 12912 |
| 247 | AACAAACCTACAAGGT | NC_045512.2 | 3′→5′ | 12898 | 12913 |
| 248 | TAACAAACCTACAAGG | NC_045512.2 | 3′→5′ | 12899 | 12914 |
| 249 | GTAACAAACCTACAAG | NC_045512.2 | 3′→5′ | 12900 | 12915 |
| 250 | TGTAACAAACCTACAA | NC_045512.2 | 3′→5′ | 12901 | 12916 |
| 251 | CTGTAACAAACCTACA | NC_045512.2 | 3′→5′ | 12902 | 12917 |
| 252 | TCTGTAACAAACCTAC | NC_045512.2 | 3′→5′ | 12903 | 12918 |
| 253 | GTCTGTAACAAACCTA | NC_045512.2 | 3′→5′ | 12904 | 12919 |
| 254 | TGTCTGTAACAAACCT | NC_045512.2 | 3′→5′ | 12905 | 12920 |
| 255 | GTGTCTGTAACAAACC | NC_045512.2 | 3′→5′ | 12906 | 12921 |
| 256 | TGTGTCTGTAACAAAC | NC_045512.2 | 3′→5′ | 12907 | 12922 |
| 257 | GTGTGTCTGTAACAAA | NC_045512.2 | 3′→5′ | 12908 | 12923 |
| 258 | GGTGTGTCTGTAACAA | NC_045512.2 | 3′→5′ | 12909 | 12924 |
| 259 | CTATTTAGGTTGTTTA | NC_045512.2 | 3′→5′ | 12966 | 12981 |
| 260 | TCTATTTAGGTTGTTT | NC_045512.2 | 3′→5′ | 12967 | 12982 |
| 261 | CTCTATTTAGGTTGTT | NC_045512.2 | 3′→5′ | 12968 | 12983 |
| 262 | CCTCTATTTAGGTTGT | NC_045512.2 | 3′→5′ | 12969 | 12984 |
| 263 | ACCTCTATTTAGGTTG | NC_045512.2 | 3′→5′ | 12970 | 12985 |
| 264 | TACCTCTATTTAGGTT | NC_045512.2 | 3′→5′ | 12971 | 12986 |
| 265 | ATACCTCTATTTAGGT | NC_045512.2 | 3′→5′ | 12972 | 12987 |
| 266 | CATACCTCTATTTAGG | NC_045512.2 | 3′→5′ | 12973 | 12988 |
| 267 | CCATACCTCTATTTAG | NC_045512.2 | 3′→5′ | 12974 | 12989 |
| 268 | ACCATACCTCTATTTA | NC_045512.2 | 3′→5′ | 12975 | 12990 |
| 269 | CAGTTGAATTGGCAGG | NC_045512.2 | 3′→5′ | 13046 | 13061 |
| 270 | ACAGTTGAATTGGCAG | NC_045512.2 | 3′→5′ | 13047 | 13062 |
| 271 | GTGTACACAACATCTT | NC_045512.2 | 3′→5′ | 13151 | 13166 |
| 272 | TGTGTACACAACATCT | NC_045512.2 | 3′→5′ | 13152 | 13167 |
| 273 | GTGTGTACACAACATC | NC_045512.2 | 3′→5′ | 13153 | 13168 |
| 274 | TGTGTGTACACAACAT | NC_045512.2 | 3′→5′ | 13154 | 13169 |
| 275 | GTGTGTGTACACAACA | NC_045512.2 | 3′→5′ | 13155 | 13170 |
| 276 | AGTGTGTGTACACAAC | NC_045512.2 | 3′→5′ | 13156 | 13171 |
| 277 | CAGTGTGTGTACACAA | NC_045512.2 | 3′→5′ | 13157 | 13172 |
| 278 | CCAGTGTGTGTACACA | NC_045512.2 | 3′→5′ | 13158 | 13173 |
| 279 | ACCAGTGTGTGTACAC | NC_045512.2 | 3′→5′ | 13159 | 13174 |
| 280 | TACCAGTGTGTGTACA | NC_045512.2 | 3′→5′ | 13160 | 13175 |
| 281 | GTACCAGTGTGTGTAC | NC_045512.2 | 3′→5′ | 13161 | 13176 |
| 282 | GGTCATTAGCACAAGT | NC_045512.2 | 3′→5′ | 13328 | 13343 |
| 283 | GGGTCATTAGCACAAG | NC_045512.2 | 3′→5′ | 13329 | 13344 |
| 284 | TAAGTGTAAAACCCAC | NC_045512.2 | 3′→5′ | 13346 | 13361 |
| 285 | GGTACAGACTGTGTTT | NC_045512.2 | 3′→5′ | 13363 | 13378 |
| 286 | CGGTACAGACTGTGTT | NC_045512.2 | 3′→5′ | 13364 | 13379 |
| 287 | ACGGTACAGACTGTGT | NC_045512.2 | 3′→5′ | 13365 | 13380 |
| 288 | GACGGTACAGACTGTG | NC_045512.2 | 3′→5′ | 13366 | 13381 |
| 289 | AGACGGTACAGACTGT | NC_045512.2 | 3′→5′ | 13367 | 13382 |
| 290 | CAGACGGTACAGACTG | NC_045512.2 | 3′→5′ | 13368 | 13383 |
| 291 | GCAGACGGTACAGACT | NC_045512.2 | 3′→5′ | 13369 | 13384 |
| 292 | CGCAGACGGTACAGAC | NC_045512.2 | 3′→5′ | 13370 | 13385 |
| 293 | CCGCAGACGGTACAGA | NC_045512.2 | 3′→5′ | 13371 | 13386 |
| 294 | CATAACCTTTCCACAT | NC_045512.2 | 3′→5′ | 13388 | 13403 |
| 295 | CCATAACCTTTCCACA | NC_045512.2 | 3′→5′ | 13389 | 13404 |
| 296 | GCCATAACCTTTCCAC | NC_045512.2 | 3′→5′ | 13390 | 13405 |
| 297 | AGCCATAACCTTTCCA | NC_045512.2 | 3′→5′ | 13391 | 13406 |
| 298 | CAGCCATAACCTTTCC | NC_045512.2 | 3′→5′ | 13392 | 13407 |
| 299 | ACAGCCATAACCTTTC | NC_045512.2 | 3′→5′ | 13393 | 13408 |
| 300 | TACAGCCATAACCTTT | NC_045512.2 | 3′→5′ | 13394 | 13409 |
| 301 | CTACAGCCATAACCTT | NC_045512.2 | 3′→5′ | 13395 | 13410 |
| 302 | ACTACAGCCATAACCT | NC_045512.2 | 3′→5′ | 13396 | 13411 |
| 303 | AACTACAGCCATAACC | NC_045512.2 | 3′→5′ | 13397 | 13412 |
| 304 | CAACTACAGCCATAAC | NC_045512.2 | 3′→5′ | 13398 | 13413 |
| 305 | ACAACTACAGCCATAA | NC_045512.2 | 3′→5′ | 13399 | 13414 |
| 306 | CACAACTACAGCCATA | NC_045512.2 | 3′→5′ | 13400 | 13415 |
| 307 | TCACAACTACAGCCAT | NC_045512.2 | 3′→5′ | 13401 | 13416 |
| 308 | AACCCGTTTAAAAACG | NC_045512.2 | 3′→5′ | 13458 | 13473 |
| 309 | AAACCCGTTTAAAAAC | NC_045512.2 | 3′→5′ | 13459 | 13474 |
| 310 | CAAACCCGTTTAAAAA | NC_045512.2 | 3′→5′ | 13460 | 13475 |
| 311 | GCAAACCCGTTTAAAA | NC_045512.2 | 3′→5′ | 13461 | 13476 |
| 312 | CGCAAACCCGTTTAAA | NC_045512.2 | 3′→5′ | 13462 | 13477 |
| 313 | CCGCAAACCCGTTTAA | NC_045512.2 | 3′→5′ | 13463 | 13478 |
| 314 | ACCGCAAACCCGTTTA | NC_045512.2 | 3′→5′ | 13464 | 13479 |
| 315 | CACCGCAAACCCGTTT | NC_045512.2 | 3′→5′ | 13465 | 13480 |
| 316 | ACACCGCAAACCCGTT | NC_045512.2 | 3′→5′ | 13466 | 13481 |
| 317 | TACACCGCAAACCCGT | NC_045512.2 | 3′→5′ | 13467 | 13482 |
| 318 | TTACACCGCAAACCCG | NC_045512.2 | 3′→5′ | 13468 | 13483 |
| 319 | CTTACACCGCAAACCC | NC_045512.2 | 3′→5′ | 13469 | 13484 |
| 320 | ACTTACACCGCAAACC | NC_045512.2 | 3′→5′ | 13470 | 13485 |
| 321 | CACTTACACCGCAAAC | NC_045512.2 | 3′→5′ | 13471 | 13486 |
| 322 | GCACTTACACCGCAAA | NC_045512.2 | 3′→5′ | 13472 | 13487 |
| 323 | TGCACTTACACCGCAA | NC_045512.2 | 3′→5′ | 13473 | 13488 |
| 324 | CTGCACTTACACCGCA | NC_045512.2 | 3′→5′ | 13474 | 13489 |
| 325 | GCTGCACTTACACCGC | NC_045512.2 | 3′→5′ | 13475 | 13490 |
| 326 | GGCTGCACTTACACCG | NC_045512.2 | 3′→5′ | 13476 | 13491 |
| 327 | GGGCTGCACTTACACC | NC_045512.2 | 3′→5′ | 13477 | 13492 |
| 328 | CGGGCTGCACTTACAC | NC_045512.2 | 3′→5′ | 13478 | 13493 |
| 329 | ACGGGCTGCACTTACA | NC_045512.2 | 3′→5′ | 13479 | 13494 |
| 330 | GACGGGCTGCACTTAC | NC_045512.2 | 3′→5′ | 13480 | 13495 |
| 331 | AGACGGGCTGCACTTA | NC_045512.2 | 3′→5′ | 13481 | 13496 |
| 332 | AAGACGGGCTGCACTT | NC_045512.2 | 3′→5′ | 13482 | 13497 |
| 333 | TAAGACGGGCTGCACT | NC_045512.2 | 3′→5′ | 13483 | 13498 |
| 334 | GTAAGACGGGCTGCAC | NC_045512.2 | 3′→5′ | 13484 | 13499 |
| 335 | TGTAAGACGGGCTGCA | NC_045512.2 | 3′→5′ | 13485 | 13500 |
| 336 | GTGTAAGACGGGCTGC | NC_045512.2 | 3′→5′ | 13486 | 13501 |
| 337 | GGTGTAAGACGGGCTG | NC_045512.2 | 3′→5′ | 13487 | 13502 |
| 338 | CGGTGTAAGACGGGCT | NC_045512.2 | 3′→5′ | 13488 | 13503 |
| 339 | ACGGTGTAAGACGGGC | NC_045512.2 | 3′→5′ | 13489 | 13504 |
| 340 | CACGGTGTAAGACGGG | NC_045512.2 | 3′→5′ | 13490 | 13505 |
| 341 | GCACGGTGTAAGACGG | NC_045512.2 | 3′→5′ | 13491 | 13506 |
| 342 | CGCACGGTGTAAGACG | NC_045512.2 | 3′→5′ | 13492 | 13507 |
| 343 | CCGCACGGTGTAAGAC | NC_045512.2 | 3′→5′ | 13493 | 13508 |
| 344 | GCCGCACGGTGTAAGA | NC_045512.2 | 3′→5′ | 13494 | 13509 |
| 345 | TGCCGCACGGTGTAAG | NC_045512.2 | 3′→5′ | 13495 | 13510 |
| 346 | GTGCCGCACGGTGTAA | NC_045512.2 | 3′→5′ | 13496 | 13511 |
| 347 | TGTGCCGCACGGTGTA | NC_045512.2 | 3′→5′ | 13497 | 13512 |
| 348 | CTGTGCCGCACGGTGT | NC_045512.2 | 3′→5′ | 13498 | 13513 |
| 349 | CCTGTGCCGCACGGTG | NC_045512.2 | 3′→5′ | 13499 | 13514 |
| 350 | GCCTGTGCCGCACGGT | NC_045512.2 | 3′→5′ | 13500 | 13515 |
| 351 | TGCCTGTGCCGCACGG | NC_045512.2 | 3′→5′ | 13501 | 13516 |
| 352 | GTGCCTGTGCCGCACG | NC_045512.2 | 3′→5′ | 13502 | 13517 |
| 353 | AGTGCCTGTGCCGCAC | NC_045512.2 | 3′→5′ | 13503 | 13518 |
| 354 | TAGTGCCTGTGCCGCA | NC_045512.2 | 3′→5′ | 13504 | 13519 |
| 355 | CTAGTGCCTGTGCCGC | NC_045512.2 | 3′→5′ | 13505 | 13520 |
| 356 | AATTAGTTTTTAGGAA | NC_045512.2 | 3′→5′ | 13582 | 13597 |
| 357 | CAATTAGTTTTTAGGA | NC_045512.2 | 3′→5′ | 13583 | 13598 |
| 358 | CTTCATGTTGGTAGTT | NC_045512.2 | 3′→5′ | 13675 | 13690 |
| 359 | TCTTCATGTTGGTAGT | NC_045512.2 | 3′→5′ | 13676 | 13691 |
| 360 | GTGGTACCATGTCACC | NC_045512.2 | 3′→5′ | 13762 | 13777 |
| 361 | TGTGGTACCATGTCAC | NC_045512.2 | 3′→5′ | 13763 | 13778 |
| 362 | ATGTGGTACCATGTCA | NC_045512.2 | 3′→5′ | 13764 | 13779 |
| 363 | TATGTGGTACCATGTC | NC_045512.2 | 3′→5′ | 13765 | 13780 |
| 364 | ATATGTGGTACCATGT | NC_045512.2 | 3′→5′ | 13766 | 13781 |
| 365 | TATATGTGGTACCATG | NC_045512.2 | 3′→5′ | 13767 | 13782 |
| 366 | ATATATGTGGTACCAT | NC_045512.2 | 3′→5′ | 13768 | 13783 |
| 367 | GATATATGTGGTACCA | NC_045512.2 | 3′→5′ | 13769 | 13784 |
| 368 | TGATATATGTGGTACC | NC_045512.2 | 3′→5′ | 13770 | 13785 |
| 369 | GTGATATATGTGGTAC | NC_045512.2 | 3′→5′ | 13771 | 13786 |
| 370 | CGTGATATATGTGGTA | NC_045512.2 | 3′→5′ | 13772 | 13787 |
| 371 | ACGTGATATATGTGGT | NC_045512.2 | 3′→5′ | 13773 | 13788 |
| 372 | GACGTGATATATGTGG | NC_045512.2 | 3′→5′ | 13774 | 13789 |
| 373 | TGACGTGATATATGTG | NC_045512.2 | 3′→5′ | 13775 | 13790 |
| 374 | CCATTGTGTATTTAGT | NC_045512.2 | 3′→5′ | 13798 | 13813 |
| 375 | GCCATTGTGTATTTAG | NC_045512.2 | 3′→5′ | 13799 | 13814 |
| 376 | ATTTAAAATAACGGTC | NC_045512.2 | 3′→5′ | 14290 | 14305 |
| 377 | TATTTAAAATAACGGT | NC_045512.2 | 3′→5′ | 14291 | 14306 |
| 378 | ATATTTAAAATAACGG | NC_045512.2 | 3′→5′ | 14292 | 14307 |
| 379 | AATATTTAAAATAACG | NC_045512.2 | 3′→5′ | 14293 | 14308 |
| 380 | CAATATTTAAAATAAC | NC_045512.2 | 3′→5′ | 14294 | 14309 |
| 381 | CCAATATTTAAAATAA | NC_045512.2 | 3′→5′ | 14295 | 14310 |
| 382 | CCCAATATTTAAAATA | NC_045512.2 | 3′→5′ | 14296 | 14311 |
| 383 | TCCCAATATTTAAAAT | NC_045512.2 | 3′→5′ | 14297 | 14312 |
| 384 | TCATCCAAACAGTTAA | NC_045512.2 | 3′→5′ | 14336 | 14351 |
| 385 | CAAAACTTGTAGGTGG | NC_045512.2 | 3′→5′ | 14404 | 14419 |
| 386 | CCAAAACTTGTAGGTG | NC_045512.2 | 3′→5′ | 14405 | 14420 |
| 387 | TCCAAAACTTGTAGGT | NC_045512.2 | 3′→5′ | 14406 | 14421 |
| 388 | GTCCAAAACTTGTAGG | NC_045512.2 | 3′→5′ | 14407 | 14422 |
| 389 | GGTCCAAAACTTGTAG | NC_045512.2 | 3′→5′ | 14408 | 14423 |
| 390 | TGGTCCAAAACTTGTA | NC_045512.2 | 3′→5′ | 14409 | 14424 |
| 391 | GTGGTCCAAAACTTGT | NC_045512.2 | 3′→5′ | 14410 | 14425 |
| 392 | AGTGGTCCAAAACTTG | NC_045512.2 | 3′→5′ | 14411 | 14426 |
| 393 | TAGTGGTCCAAAACTT | NC_045512.2 | 3′→5′ | 14412 | 14427 |
| 394 | CTAGTGGTCCAAAACT | NC_045512.2 | 3′→5′ | 14413 | 14428 |
| 395 | ACTAGTGGTCCAAAAC | NC_045512.2 | 3′→5′ | 14414 | 14429 |
| 396 | CATCCTGATTATGTAC | NC_045512.2 | 3′→5′ | 14500 | 14515 |
| 397 | ACATCCTGATTATGTA | NC_045512.2 | 3′→5′ | 14501 | 14516 |
| 398 | TACATCCTGATTATGT | NC_045512.2 | 3′→5′ | 14502 | 14517 |
| 399 | TTACATCCTGATTATG | NC_045512.2 | 3′→5′ | 14503 | 14518 |
| 400 | TTTACATCCTGATTAT | NC_045512.2 | 3′→5′ | 14504 | 14519 |
| 401 | GTTTACATCCTGATTA | NC_045512.2 | 3′→5′ | 14505 | 14520 |
| 402 | AGTTTACATCCTGATT | NC_045512.2 | 3′→5′ | 14506 | 14521 |
| 403 | AAGTTTACATCCTGAT | NC_045512.2 | 3′→5′ | 14507 | 14522 |
| 404 | TAAGTTTACATCCTGA | NC_045512.2 | 3′→5′ | 14508 | 14523 |
| 405 | GTAAGTTTACATCCTG | NC_045512.2 | 3′→5′ | 14509 | 14524 |
| 406 | TGTAAGTTTACATCCT | NC_045512.2 | 3′→5′ | 14510 | 14525 |
| 407 | ATGTAAGTTTACATCC | NC_045512.2 | 3′→5′ | 14511 | 14526 |
| 408 | TATGTAAGTTTACATC | NC_045512.2 | 3′→5′ | 14512 | 14527 |
| 409 | CTATGTAAGTTTACAT | NC_045512.2 | 3′→5′ | 14513 | 14528 |
| 410 | GCTATGTAAGTTTACA | NC_045512.2 | 3′→5′ | 14514 | 14529 |
| 411 | AGCTATGTAAGTTTAC | NC_045512.2 | 3′→5′ | 14515 | 14530 |
| 412 | GAGCTATGTAAGTTTA | NC_045512.2 | 3′→5′ | 14516 | 14531 |
| 413 | TAGTGCGTTTATCTAG | NC_045512.2 | 3′→5′ | 14605 | 14620 |
| 414 | GTAGTGCGTTTATCTA | NC_045512.2 | 3′→5′ | 14606 | 14621 |
| 415 | CAGCTACTGAAAAGCA | NC_045512.2 | 3′→5′ | 14623 | 14638 |
| 416 | GCAGCTACTGAAAAGC | NC_045512.2 | 3′→5′ | 14624 | 14639 |
| 417 | TGCAGCTACTGAAAAG | NC_045512.2 | 3′→5′ | 14625 | 14640 |
| 418 | GTGCAGCTACTGAAAA | NC_045512.2 | 3′→5′ | 14626 | 14641 |
| 419 | AGTGCAGCTACTGAAA | NC_045512.2 | 3′→5′ | 14627 | 14642 |
| 420 | TTTGAAAAGCAACATT | NC_045512.2 | 3′→5′ | 14650 | 14665 |
| 421 | GTTTGAAAAGCAACAT | NC_045512.2 | 3′→5′ | 14651 | 14666 |
| 422 | AGTTTGAAAAGCAACA | NC_045512.2 | 3′→5′ | 14652 | 14667 |
| 423 | CAGTTTGAAAAGCAAC | NC_045512.2 | 3′→5′ | 14653 | 14668 |
| 424 | ACAGTTTGAAAAGCAA | NC_045512.2 | 3′→5′ | 14654 | 14669 |
| 425 | GACAGTTTGAAAAGCA | NC_045512.2 | 3′→5′ | 14655 | 14670 |
| 426 | TGACAGTTTGAAAAGC | NC_045512.2 | 3′→5′ | 14656 | 14671 |
| 427 | TTGACAGTTTGAAAAG | NC_045512.2 | 3′→5′ | 14657 | 14672 |
| 428 | TTTGACAGTTTGAAAA | NC_045512.2 | 3′→5′ | 14658 | 14673 |
| 429 | GTTTGACAGTTTGAAA | NC_045512.2 | 3′→5′ | 14659 | 14674 |
| 430 | GGTTTGACAGTTTGAA | NC_045512.2 | 3′→5′ | 14660 | 14675 |
| 431 | GGGTTTGACAGTTTGA | NC_045512.2 | 3′→5′ | 14661 | 14676 |
| 432 | CGGGTTTGACAGTTTG | NC_045512.2 | 3′→5′ | 14662 | 14677 |
| 433 | CCGGGTTTGACAGTTT | NC_045512.2 | 3′→5′ | 14663 | 14678 |
| 434 | ACCGGGTTTGACAGTT | NC_045512.2 | 3′→5′ | 14664 | 14679 |
| 435 | TACCGGGTTTGACAGT | NC_045512.2 | 3′→5′ | 14665 | 14680 |
| 436 | TTACCGGGTTTGACAG | NC_045512.2 | 3′→5′ | 14666 | 14681 |
| 437 | ATTACCGGGTTTGACA | NC_045512.2 | 3′→5′ | 14667 | 14682 |
| 438 | AATTACCGGGTTTGAC | NC_045512.2 | 3′→5′ | 14668 | 14683 |
| 439 | AAATTACCGGGTTTGA | NC_045512.2 | 3′→5′ | 14669 | 14684 |
| 440 | AAAATTACCGGGTTTG | NC_045512.2 | 3′→5′ | 14670 | 14685 |
| 441 | TAAAATTACCGGGTTT | NC_045512.2 | 3′→5′ | 14671 | 14686 |
| 442 | TTAAAATTACCGGGTT | NC_045512.2 | 3′→5′ | 14672 | 14687 |
| 443 | ACACAGCAAAGTCATA | NC_045512.2 | 3′→5′ | 14698 | 14713 |
| 444 | GACACAGCAAAGTCAT | NC_045512.2 | 3′→5′ | 14699 | 14714 |
| 445 | AGACACAGCAAAGTCA | NC_045512.2 | 3′→5′ | 14700 | 14715 |
| 446 | TAGACACAGCAAAGTC | NC_045512.2 | 3′→5′ | 14701 | 14716 |
| 447 | TTAGACACAGCAAAGT | NC_045512.2 | 3′→5′ | 14702 | 14717 |
| 448 | TTCCTTCCTTAAAGAA | NC_045512.2 | 3′→5′ | 14722 | 14737 |
| 449 | CTTCCTTCCTTAAAGA | NC_045512.2 | 3′→5′ | 14723 | 14738 |
| 450 | ACTTCCTTCCTTAAAG | NC_045512.2 | 3′→5′ | 14724 | 14739 |
| 451 | AACTTCCTTCCTTAAA | NC_045512.2 | 3′→5′ | 14725 | 14740 |
| 452 | GAACTTCCTTCCTTAA | NC_045512.2 | 3′→5′ | 14726 | 14741 |
| 453 | AGAACTTCCTTCCTTA | NC_045512.2 | 3′→5′ | 14727 | 14742 |
| 454 | CAGAACTTCCTTCCTT | NC_045512.2 | 3′→5′ | 14728 | 14743 |
| 455 | ACAGAACTTCCTTCCT | NC_045512.2 | 3′→5′ | 14729 | 14744 |
| 456 | AACAGAACTTCCTTCC | NC_045512.2 | 3′→5′ | 14730 | 14745 |
| 457 | CAACAGAACTTCCTTC | NC_045512.2 | 3′→5′ | 14731 | 14746 |
| 458 | TCAACAGAACTTCCTT | NC_045512.2 | 3′→5′ | 14732 | 14747 |
| 459 | TTCAACAGAACTTCCT | NC_045512.2 | 3′→5′ | 14733 | 14748 |
| 460 | AAGAAGAAGTGTTTTA | NC_045512.2 | 3′→5′ | 14750 | 14765 |
| 461 | AAAGAAGAAGTGTTTT | NC_045512.2 | 3′→5′ | 14751 | 14766 |
| 462 | CAAAGAAGAAGTGTTT | NC_045512.2 | 3′→5′ | 14752 | 14767 |
| 463 | GCAAAGAAGAAGTGTT | NC_045512.2 | 3′→5′ | 14753 | 14768 |
| 464 | AGCAAAGAAGAAGTGT | NC_045512.2 | 3′→5′ | 14754 | 14769 |
| 465 | GAGCAAAGAAGAAGTG | NC_045512.2 | 3′→5′ | 14755 | 14770 |
| 466 | TGAGCAAAGAAGAAGT | NC_045512.2 | 3′→5′ | 14756 | 14771 |
| 467 | CTGAGCAAAGAAGAAG | NC_045512.2 | 3′→5′ | 14757 | 14772 |
| 468 | CCTGAGCAAAGAAGAA | NC_045512.2 | 3′→5′ | 14758 | 14773 |
| 469 | TCCTGAGCAAAGAAGA | NC_045512.2 | 3′→5′ | 14759 | 14774 |
| 470 | ATCCTGAGCAAAGAAG | NC_045512.2 | 3′→5′ | 14760 | 14775 |
| 471 | CATCCTGAGCAAAGAA | NC_045512.2 | 3′→5′ | 14761 | 14776 |
| 472 | CCATCCTGAGCAAAGA | NC_045512.2 | 3′→5′ | 14762 | 14777 |
| 473 | TATCACACATTGTTGG | NC_045512.2 | 3′→5′ | 14821 | 14836 |
| 474 | ATATCACACATTGTTG | NC_045512.2 | 3′→5′ | 14822 | 14837 |
| 475 | GATATCACACATTGTT | NC_045512.2 | 3′→5′ | 14823 | 14838 |
| 476 | TGATATCACACATTGT | NC_045512.2 | 3′→5′ | 14824 | 14839 |
| 477 | CTGATATCACACATTG | NC_045512.2 | 3′→5′ | 14825 | 14840 |
| 478 | TCTGATATCACACATT | NC_045512.2 | 3′→5′ | 14826 | 14841 |
| 479 | GTCTGATATCACACAT | NC_045512.2 | 3′→5′ | 14827 | 14842 |
| 480 | TGTCTGATATCACACA | NC_045512.2 | 3′→5′ | 14828 | 14843 |
| 481 | TTGTCTGATATCACAC | NC_045512.2 | 3′→5′ | 14829 | 14844 |
| 482 | GTTGTCTGATATCACA | NC_045512.2 | 3′→5′ | 14830 | 14845 |
| 483 | AGTTGTCTGATATCAC | NC_045512.2 | 3′→5′ | 14831 | 14846 |
| 484 | CAACAACTTCAACTAC | NC_045512.2 | 3′→5′ | 14854 | 14869 |
| 485 | TCAACAACTTCAACTA | NC_045512.2 | 3′→5′ | 14855 | 14870 |
| 486 | ATCAACAACTTCAACT | NC_045512.2 | 3′→5′ | 14856 | 14871 |
| 487 | TATCAACAACTTCAAC | NC_045512.2 | 3′→5′ | 14857 | 14872 |
| 488 | TTATCAACAACTTCAA | NC_045512.2 | 3′→5′ | 14858 | 14873 |
| 489 | CGTAACAATCAAAGTA | NC_045512.2 | 3′→5′ | 14875 | 14890 |
| 490 | TCGTAACAATCAAAGT | NC_045512.2 | 3′→5′ | 14876 | 14891 |
| 491 | ATCGTAACAATCAAAG | NC_045512.2 | 3′→5′ | 14877 | 14892 |
| 492 | CATCGTAACAATCAAA | NC_045512.2 | 3′→5′ | 14878 | 14893 |
| 493 | CCATCGTAACAATCAA | NC_045512.2 | 3′→5′ | 14879 | 14894 |
| 494 | ACCATCGTAACAATCA | NC_045512.2 | 3′→5′ | 14880 | 14895 |
| 495 | CACCATCGTAACAATC | NC_045512.2 | 3′→5′ | 14881 | 14896 |
| 496 | CCACCATCGTAACAAT | NC_045512.2 | 3′→5′ | 14882 | 14897 |
| 497 | GCCACCATCGTAACAA | NC_045512.2 | 3′→5′ | 14883 | 14898 |
| 498 | AGCCACCATCGTAACA | NC_045512.2 | 3′→5′ | 14884 | 14899 |
| 499 | CAGCCACCATCGTAAC | NC_045512.2 | 3′→5′ | 14885 | 14900 |
| 500 | ACAGCCACCATCGTAA | NC_045512.2 | 3′→5′ | 14886 | 14901 |
| 501 | TACAGCCACCATCGTA | NC_045512.2 | 3′→5′ | 14887 | 14902 |
| 502 | ATACAGCCACCATCGT | NC_045512.2 | 3′→5′ | 14888 | 14903 |
| 503 | TAGCCTTACCCCATTT | NC_045512.2 | 3′→5′ | 14962 | 14977 |
| 504 | CTAGCCTTACCCCATT | NC_045512.2 | 3′→5′ | 14963 | 14978 |
| 505 | TCTAGCCTTACCCCAT | NC_045512.2 | 3′→5′ | 14964 | 14979 |
| 506 | GTCTAGCCTTACCCCA | NC_045512.2 | 3′→5′ | 14965 | 14980 |
| 507 | AGTCTAGCCTTACCCC | NC_045512.2 | 3′→5′ | 14966 | 14981 |
| 508 | AAGTCTAGCCTTACCC | NC_045512.2 | 3′→5′ | 14967 | 14982 |
| 509 | AAAGTCTAGCCTTACC | NC_045512.2 | 3′→5′ | 14968 | 14983 |
| 510 | TAAAGTCTAGCCTTAC | NC_045512.2 | 3′→5′ | 14969 | 14984 |
| 511 | ATAAAGTCTAGCCTTA | NC_045512.2 | 3′→5′ | 14970 | 14985 |
| 512 | AATAAAGTCTAGCCTT | NC_045512.2 | 3′→5′ | 14971 | 14986 |
| 513 | TAATAAAGTCTAGCCT | NC_045512.2 | 3′→5′ | 14972 | 14987 |
| 514 | ATAATAAAGTCTAGCC | NC_045512.2 | 3′→5′ | 14973 | 14988 |
| 515 | CATAATAAAGTCTAGC | NC_045512.2 | 3′→5′ | 14974 | 14989 |
| 516 | TCATAATAAAGTCTAG | NC_045512.2 | 3′→5′ | 14975 | 14990 |
| 517 | CCTCATAACTCATTGA | NC_045512.2 | 3′→5′ | 14992 | 15007 |
| 518 | TCCTCATAACTCATTG | NC_045512.2 | 3′→5′ | 14993 | 15008 |
| 519 | ATCCTCATAACTCATT | NC_045512.2 | 3′→5′ | 14994 | 15009 |
| 520 | GATCCTCATAACTCAT | NC_045512.2 | 3′→5′ | 14995 | 15010 |
| 521 | TGATCCTCATAACTCA | NC_045512.2 | 3′→5′ | 14996 | 15011 |
| 522 | TTGATCCTCATAACTC | NC_045512.2 | 3′→5′ | 14997 | 15012 |
| 523 | CTTGATCCTCATAACT | NC_045512.2 | 3′→5′ | 14998 | 15013 |
| 524 | TCTTGATCCTCATAAC | NC_045512.2 | 3′→5′ | 14999 | 15014 |
| 525 | ATCTTGATCCTCATAA | NC_045512.2 | 3′→5′ | 15000 | 15015 |
| 526 | CATCTTGATCCTCATA | NC_045512.2 | 3′→5′ | 15001 | 15016 |
| 527 | GCATCTTGATCCTCAT | NC_045512.2 | 3′→5′ | 15002 | 15017 |
| 528 | TGCATCTTGATCCTCA | NC_045512.2 | 3′→5′ | 15003 | 15018 |
| 529 | GTGCATCTTGATCCTC | NC_045512.2 | 3′→5′ | 15004 | 15019 |
| 530 | AGTGCATCTTGATCCT | NC_045512.2 | 3′→5′ | 15005 | 15020 |
| 531 | TAGGGATGACATTACG | NC_045512.2 | 3′→5′ | 15037 | 15052 |
| 532 | GTAGGGATGACATTAC | NC_045512.2 | 3′→5′ | 15038 | 15053 |
| 533 | GATTCATTTGAGTTAT | NC_045512.2 | 3′→5′ | 15055 | 15070 |
| 534 | AGATTCATTTGAGTTA | NC_045512.2 | 3′→5′ | 15056 | 15071 |
| 535 | AAGATTCATTTGAGTT | NC_045512.2 | 3′→5′ | 15057 | 15072 |
| 536 | TAAGATTCATTTGAGT | NC_045512.2 | 3′→5′ | 15058 | 15073 |
| 537 | TTAAGATTCATTTGAG | NC_045512.2 | 3′→5′ | 15059 | 15074 |
| 538 | CTTAAGATTCATTTGA | NC_045512.2 | 3′→5′ | 15060 | 15075 |
| 539 | ACTTAAGATTCATTTG | NC_045512.2 | 3′→5′ | 15061 | 15076 |
| 540 | TACTTAAGATTCATTT | NC_045512.2 | 3′→5′ | 15062 | 15077 |
| 541 | ATACTTAAGATTCATT | NC_045512.2 | 3′→5′ | 15063 | 15078 |
| 542 | CATACTTAAGATTCAT | NC_045512.2 | 3′→5′ | 15064 | 15079 |
| 543 | GCATACTTAAGATTCA | NC_045512.2 | 3′→5′ | 15065 | 15080 |
| 544 | GGCATACTTAAGATTC | NC_045512.2 | 3′→5′ | 15066 | 15081 |
| 545 | TGGCATACTTAAGATT | NC_045512.2 | 3′→5′ | 15067 | 15082 |
| 546 | ATGGCATACTTAAGAT | NC_045512.2 | 3′→5′ | 15068 | 15083 |
| 547 | AATGGCATACTTAAGA | NC_045512.2 | 3′→5′ | 15069 | 15084 |
| 548 | TAATGGCATACTTAAG | NC_045512.2 | 3′→5′ | 15070 | 15085 |
| 549 | CTAATGGCATACTTAA | NC_045512.2 | 3′→5′ | 15071 | 15086 |
| 550 | ACTAATGGCATACTTA | NC_045512.2 | 3′→5′ | 15072 | 15087 |
| 551 | CACTAATGGCATACTT | NC_045512.2 | 3′→5′ | 15073 | 15088 |
| 552 | GCACTAATGGCATACT | NC_045512.2 | 3′→5′ | 15074 | 15089 |
| 553 | TGCACTAATGGCATAC | NC_045512.2 | 3′→5′ | 15075 | 15090 |
| 554 | TTGCACTAATGGCATA | NC_045512.2 | 3′→5′ | 15076 | 15091 |
| 555 | TTTGCACTAATGGCAT | NC_045512.2 | 3′→5′ | 15077 | 15092 |
| 556 | CTTTGCACTAATGGCA | NC_045512.2 | 3′→5′ | 15078 | 15093 |
| 557 | TCTTTGCACTAATGGC | NC_045512.2 | 3′→5′ | 15079 | 15094 |
| 558 | TTCTTTGCACTAATGG | NC_045512.2 | 3′→5′ | 15080 | 15095 |
| 559 | ATTCTTTGCACTAATG | NC_045512.2 | 3′→5′ | 15081 | 15096 |
| 560 | TATTCTTTGCACTAAT | NC_045512.2 | 3′→5′ | 15082 | 15097 |
| 561 | CTATTCTTTGCACTAA | NC_045512.2 | 3′→5′ | 15083 | 15098 |
| 562 | TCTATTCTTTGCACTA | NC_045512.2 | 3′→5′ | 15084 | 15099 |
| 563 | CTCTATTCTTTGCACT | NC_045512.2 | 3′→5′ | 15085 | 15100 |
| 564 | GCTCTATTCTTTGCAC | NC_045512.2 | 3′→5′ | 15086 | 15101 |
| 565 | AGCTCTATTCTTTGCA | NC_045512.2 | 3′→5′ | 15087 | 15102 |
| 566 | GAGCTCTATTCTTTGC | NC_045512.2 | 3′→5′ | 15088 | 15103 |
| 567 | CGAGCTCTATTCTTTG | NC_045512.2 | 3′→5′ | 15089 | 15104 |
| 568 | GCGAGCTCTATTCTTT | NC_045512.2 | 3′→5′ | 15090 | 15105 |
| 569 | TGCGAGCTCTATTCTT | NC_045512.2 | 3′→5′ | 15091 | 15106 |
| 570 | GTGCGAGCTCTATTCT | NC_045512.2 | 3′→5′ | 15092 | 15107 |
| 571 | GGTGCGAGCTCTATTC | NC_045512.2 | 3′→5′ | 15093 | 15108 |
| 572 | CGGTGCGAGCTCTATT | NC_045512.2 | 3′→5′ | 15094 | 15109 |
| 573 | ACGGTGCGAGCTCTAT | NC_045512.2 | 3′→5′ | 15095 | 15110 |
| 574 | TACGGTGCGAGCTCTA | NC_045512.2 | 3′→5′ | 15096 | 15111 |
| 575 | CTACGGTGCGAGCTCT | NC_045512.2 | 3′→5′ | 15097 | 15112 |
| 576 | GCTACGGTGCGAGCTC | NC_045512.2 | 3′→5′ | 15098 | 15113 |
| 577 | AGCTACGGTGCGAGCT | NC_045512.2 | 3′→5′ | 15099 | 15114 |
| 578 | CAGCTACGGTGCGAGC | NC_045512.2 | 3′→5′ | 15100 | 15115 |
| 579 | CCAGCTACGGTGCGAG | NC_045512.2 | 3′→5′ | 15101 | 15116 |
| 580 | ACCAGCTACGGTGCGA | NC_045512.2 | 3′→5′ | 15102 | 15117 |
| 581 | CACCAGCTACGGTGCG | NC_045512.2 | 3′→5′ | 15103 | 15118 |
| 582 | ACACCAGCTACGGTGC | NC_045512.2 | 3′→5′ | 15104 | 15119 |
| 583 | GACACCAGCTACGGTG | NC_045512.2 | 3′→5′ | 15105 | 15120 |
| 584 | AGACACCAGCTACGGT | NC_045512.2 | 3′→5′ | 15106 | 15121 |
| 585 | GAGACACCAGCTACGG | NC_045512.2 | 3′→5′ | 15107 | 15122 |
| 586 | AGAGACACCAGCTACG | NC_045512.2 | 3′→5′ | 15108 | 15123 |
| 587 | TAGAGACACCAGCTAC | NC_045512.2 | 3′→5′ | 15109 | 15124 |
| 588 | ATAGAGACACCAGCTA | NC_045512.2 | 3′→5′ | 15110 | 15125 |
| 589 | GATAGAGACACCAGCT | NC_045512.2 | 3′→5′ | 15111 | 15126 |
| 590 | AGATAGAGACACCAGC | NC_045512.2 | 3′→5′ | 15112 | 15127 |
| 591 | CAGATAGAGACACCAG | NC_045512.2 | 3′→5′ | 15113 | 15128 |
| 592 | ACAGATAGAGACACCA | NC_045512.2 | 3′→5′ | 15114 | 15129 |
| 593 | TACAGATAGAGACACC | NC_045512.2 | 3′→5′ | 15115 | 15130 |
| 594 | CTACAGATAGAGACAC | NC_045512.2 | 3′→5′ | 15116 | 15131 |
| 595 | ACTACAGATAGAGACA | NC_045512.2 | 3′→5′ | 15117 | 15132 |
| 596 | TACTACAGATAGAGAC | NC_045512.2 | 3′→5′ | 15118 | 15133 |
| 597 | GTACTACAGATAGAGA | NC_045512.2 | 3′→5′ | 15119 | 15134 |
| 598 | AGTACTACAGATAGAG | NC_045512.2 | 3′→5′ | 15120 | 15135 |
| 599 | TAGTACTACAGATAGA | NC_045512.2 | 3′→5′ | 15121 | 15136 |
| 600 | ATAGTACTACAGATAG | NC_045512.2 | 3′→5′ | 15122 | 15137 |
| 601 | CATAGTACTACAGATA | NC_045512.2 | 3′→5′ | 15123 | 15138 |
| 602 | TCATAGTACTACAGAT | NC_045512.2 | 3′→5′ | 15124 | 15139 |
| 603 | GTCATAGTACTACAGA | NC_045512.2 | 3′→5′ | 15125 | 15140 |
| 604 | GATGAAACTGTCTATT | NC_045512.2 | 3′→5′ | 15142 | 15157 |
| 605 | TGATGAAACTGTCTAT | NC_045512.2 | 3′→5′ | 15143 | 15158 |
| 606 | TAGTGGCGGCTATTGA | NC_045512.2 | 3′→5′ | 15172 | 15187 |
| 607 | CTAGTGGCGGCTATTG | NC_045512.2 | 3′→5′ | 15173 | 15188 |
| 608 | TCTAGTGGCGGCTATT | NC_045512.2 | 3′→5′ | 15174 | 15189 |
| 609 | CTCTAGTGGCGGCTAT | NC_045512.2 | 3′→5′ | 15175 | 15190 |
| 610 | CCTCTAGTGGCGGCTA | NC_045512.2 | 3′→5′ | 15176 | 15191 |
| 611 | TCCTCTAGTGGCGGCT | NC_045512.2 | 3′→5′ | 15177 | 15192 |
| 612 | CTCCTCTAGTGGCGGC | NC_045512.2 | 3′→5′ | 15178 | 15193 |
| 613 | GCTCCTCTAGTGGCGG | NC_045512.2 | 3′→5′ | 15179 | 15194 |
| 614 | AGCTCCTCTAGTGGCG | NC_045512.2 | 3′→5′ | 15180 | 15195 |
| 615 | TAGCTCCTCTAGTGGC | NC_045512.2 | 3′→5′ | 15181 | 15196 |
| 616 | GTAGCTCCTCTAGTGG | NC_045512.2 | 3′→5′ | 15182 | 15197 |
| 617 | AGTAGCTCCTCTAGTG | NC_045512.2 | 3′→5′ | 15183 | 15198 |
| 618 | CAGTAGCTCCTCTAGT | NC_045512.2 | 3′→5′ | 15184 | 15199 |
| 619 | ACAGTAGCTCCTCTAG | NC_045512.2 | 3′→5′ | 15185 | 15200 |
| 620 | TGCTTGTTCCAATTAC | NC_045512.2 | 3′→5′ | 15202 | 15217 |
| 621 | TTGCTTGTTCCAATTA | NC_045512.2 | 3′→5′ | 15203 | 15218 |
| 622 | AAACAGTTTTTAACAT | NC_045512.2 | 3′→5′ | 15241 | 15256 |
| 623 | TAAACAGTTTTTAACA | NC_045512.2 | 3′→5′ | 15242 | 15257 |
| 624 | CCCAACCCATAAGGTG | NC_045512.2 | 3′→5′ | 15277 | 15292 |
| 625 | TCCCAACCCATAAGGT | NC_045512.2 | 3′→5′ | 15278 | 15293 |
| 626 | TGTTAGGCATGGCTCT | NC_045512.2 | 3′→5′ | 15310 | 15325 |
| 627 | ATGTTAGGCATGGCTC | NC_045512.2 | 3′→5′ | 15311 | 15326 |
| 628 | CATGTTAGGCATGGCT | NC_045512.2 | 3′→5′ | 15312 | 15327 |
| 629 | GCATGTTAGGCATGGC | NC_045512.2 | 3′→5′ | 15313 | 15328 |
| 630 | AGCATGTTAGGCATGG | NC_045512.2 | 3′→5′ | 15314 | 15329 |
| 631 | AAGCATGTTAGGCATG | NC_045512.2 | 3′→5′ | 15315 | 15330 |
| 632 | TAAGCATGTTAGGCAT | NC_045512.2 | 3′→5′ | 15316 | 15331 |
| 633 | CTAAGCATGTTAGGCA | NC_045512.2 | 3′→5′ | 15317 | 15332 |
| 634 | TGCGAGCAAGAACAAG | NC_045512.2 | 3′→5′ | 15346 | 15361 |
| 635 | TTGCGAGCAAGAACAA | NC_045512.2 | 3′→5′ | 15347 | 15362 |
| 636 | TTTGCGAGCAAGAACA | NC_045512.2 | 3′→5′ | 15348 | 15363 |
| 637 | GTTTGCGAGCAAGAAC | NC_045512.2 | 3′→5′ | 15349 | 15364 |
| 638 | TGTTTGCGAGCAAGAA | NC_045512.2 | 3′→5′ | 15350 | 15365 |
| 639 | ATGTTTGCGAGCAAGA | NC_045512.2 | 3′→5′ | 15351 | 15366 |
| 640 | TATGTTTGCGAGCAAG | NC_045512.2 | 3′→5′ | 15352 | 15367 |
| 641 | CGCCACACATGACCAT | NC_045512.2 | 3′→5′ | 15436 | 15451 |
| 642 | CCGCCACACATGACCA | NC_045512.2 | 3′→5′ | 15437 | 15452 |
| 643 | TAGCATAAGCAGTTGT | NC_045512.2 | 3′→5′ | 15496 | 15511 |
| 644 | TTAGCATAAGCAGTTG | NC_045512.2 | 3′→5′ | 15497 | 15512 |
| 645 | ATTAGCATAAGCAGTT | NC_045512.2 | 3′→5′ | 15498 | 15513 |
| 646 | TATTAGCATAAGCAGT | NC_045512.2 | 3′→5′ | 15499 | 15514 |
| 647 | CTATTAGCATAAGCAG | NC_045512.2 | 3′→5′ | 15500 | 15515 |
| 648 | ACTATTAGCATAAGCA | NC_045512.2 | 3′→5′ | 15501 | 15516 |
| 649 | CACTATTAGCATAAGC | NC_045512.2 | 3′→5′ | 15502 | 15517 |
| 650 | ACACTATTAGCATAAG | NC_045512.2 | 3′→5′ | 15503 | 15518 |
| 651 | CTTGACAAATGTTAAA | NC_045512.2 | 3′→5′ | 15520 | 15535 |
| 652 | GCTTGACAAATGTTAA | NC_045512.2 | 3′→5′ | 15521 | 15536 |
| 653 | TATAGAGACACTCATA | NC_045512.2 | 3′→5′ | 15622 | 15637 |
| 654 | CTATAGAGACACTCAT | NC_045512.2 | 3′→5′ | 15623 | 15638 |
| 655 | TCTATAGAGACACTCA | NC_045512.2 | 3′→5′ | 15624 | 15639 |
| 656 | TTCTATAGAGACACTC | NC_045512.2 | 3′→5′ | 15625 | 15640 |
| 657 | TTTCTATAGAGACACT | NC_045512.2 | 3′→5′ | 15626 | 15641 |
| 658 | ATTTCTATAGAGACAC | NC_045512.2 | 3′→5′ | 15627 | 15642 |
| 659 | TATTTCTATAGAGACA | NC_045512.2 | 3′→5′ | 15628 | 15643 |
| 660 | CTATTTCTATAGAGAC | NC_045512.2 | 3′→5′ | 15629 | 15644 |
| 661 | GAGAAATGTTTACGCA | NC_045512.2 | 3′→5′ | 15686 | 15701 |
| 662 | GGTCAGTCTCAGTCCA | NC_045512.2 | 3′→5′ | 15838 | 15853 |
| 663 | AGGTCAGTCTCAGTCC | NC_045512.2 | 3′→5′ | 15839 | 15854 |
| 664 | AAGGTCAGTCTCAGTC | NC_045512.2 | 3′→5′ | 15840 | 15855 |
| 665 | TAAGGTCAGTCTCAGT | NC_045512.2 | 3′→5′ | 15841 | 15856 |
| 666 | GTAAGGTCAGTCTCAG | NC_045512.2 | 3′→5′ | 15842 | 15857 |
| 667 | AGTAAGGTCAGTCTCA | NC_045512.2 | 3′→5′ | 15843 | 15858 |
| 668 | TAGTAAGGTCAGTCTC | NC_045512.2 | 3′→5′ | 15844 | 15859 |
| 669 | TTAGTAAGGTCAGTCT | NC_045512.2 | 3′→5′ | 15845 | 15860 |
| 670 | TTTAGTAAGGTCAGTC | NC_045512.2 | 3′→5′ | 15846 | 15861 |
| 671 | CTTTAGTAAGGTCAGT | NC_045512.2 | 3′→5′ | 15847 | 15862 |
| 672 | CCTTTAGTAAGGTCAG | NC_045512.2 | 3′→5′ | 15848 | 15863 |
| 673 | TCCTTTAGTAAGGTCA | NC_045512.2 | 3′→5′ | 15849 | 15864 |
| 674 | GTCCTTTAGTAAGGTC | NC_045512.2 | 3′→5′ | 15850 | 15865 |
| 675 | GGTCCTTTAGTAAGGT | NC_045512.2 | 3′→5′ | 15851 | 15866 |
| 676 | AGGTCCTTTAGTAAGG | NC_045512.2 | 3′→5′ | 15852 | 15867 |
| 677 | GAGGTCCTTTAGTAAG | NC_045512.2 | 3′→5′ | 15853 | 15868 |
| 678 | TGAGGTCCTTTAGTAA | NC_045512.2 | 3′→5′ | 15854 | 15869 |
| 679 | TAACTAGCATTGTATG | NC_045512.2 | 3′→5′ | 15886 | 15901 |
| 680 | TTAACTAGCATTGTAT | NC_045512.2 | 3′→5′ | 15887 | 15902 |
| 681 | TTTAACTAGCATTGTA | NC_045512.2 | 3′→5′ | 15888 | 15903 |
| 682 | GTTTAACTAGCATTGT | NC_045512.2 | 3′→5′ | 15889 | 15904 |
| 683 | TGTTTAACTAGCATTG | NC_045512.2 | 3′→5′ | 15890 | 15905 |
| 684 | ATGGATCTGGGTAAGG | NC_045512.2 | 3′→5′ | 15928 | 15943 |
| 685 | GATGGATCTGGGTAAG | NC_045512.2 | 3′→5′ | 15929 | 15944 |
| 686 | GTGTACCATCTGTTTT | NC_045512.2 | 3′→5′ | 15985 | 16000 |
| 687 | AGTGTACCATCTGTTT | NC_045512.2 | 3′→5′ | 15986 | 16001 |
| 688 | AAGTGTACCATCTGTT | NC_045512.2 | 3′→5′ | 15987 | 16002 |
| 689 | TAAGTGTACCATCTGT | NC_045512.2 | 3′→5′ | 15988 | 16003 |
| 690 | ATAAGTGTACCATCTG | NC_045512.2 | 3′→5′ | 15989 | 16004 |
| 691 | CATAAGTGTACCATCT | NC_045512.2 | 3′→5′ | 15990 | 16005 |
| 692 | TCATAAGTGTACCATC | NC_045512.2 | 3′→5′ | 15991 | 16006 |
| 693 | ATCATAAGTGTACCAT | NC_045512.2 | 3′→5′ | 15992 | 16007 |
| 694 | AATCATAAGTGTACCA | NC_045512.2 | 3′→5′ | 15993 | 16008 |
| 695 | CAATCATAAGTGTACC | NC_045512.2 | 3′→5′ | 15994 | 16009 |
| 696 | TCAATCATAAGTGTAC | NC_045512.2 | 3′→5′ | 15995 | 16010 |
| 697 | CATACTCCTGATTAGG | NC_045512.2 | 3′→5′ | 16057 | 16072 |
| 698 | GCATACTCCTGATTAG | NC_045512.2 | 3′→5′ | 16058 | 16073 |
| 699 | AGCATACTCCTGATTA | NC_045512.2 | 3′→5′ | 16059 | 16074 |
| 700 | CAGCATACTCCTGATT | NC_045512.2 | 3′→5′ | 16060 | 16075 |
| 701 | TCAGCATACTCCTGAT | NC_045512.2 | 3′→5′ | 16061 | 16076 |
| 702 | ATCAGCATACTCCTGA | NC_045512.2 | 3′→5′ | 16062 | 16077 |
| 703 | CATCAGCATACTCCTG | NC_045512.2 | 3′→5′ | 16063 | 16078 |
| 704 | ACATCAGCATACTCCT | NC_045512.2 | 3′→5′ | 16064 | 16079 |
| 705 | AAAACTCAGGTTCCCA | NC_045512.2 | 3′→5′ | 16186 | 16201 |
| 706 | TAAAACTCAGGTTCCC | NC_045512.2 | 3′→5′ | 16187 | 16202 |
| 707 | ATAAAACTCAGGTTCC | NC_045512.2 | 3′→5′ | 16188 | 16203 |
| 708 | CATAAAACTCAGGTTC | NC_045512.2 | 3′→5′ | 16189 | 16204 |
| 709 | TCATAAAACTCAGGTT | NC_045512.2 | 3′→5′ | 16190 | 16205 |
| 710 | TAGCTCATACCTCCTA | NC_045512.2 | 3′→5′ | 16430 | 16445 |
| 711 | ATAGCTCATACCTCCT | NC_045512.2 | 3′→5′ | 16431 | 16446 |
| 712 | AATAGCTCATACCTCC | NC_045512.2 | 3′→5′ | 16432 | 16447 |
| 713 | TAATAGCTCATACCTC | NC_045512.2 | 3′→5′ | 16433 | 16448 |
| 714 | ATGGAAAACTAATGGG | NC_045512.2 | 3′→5′ | 16468 | 16483 |
| 715 | AATGGAAAACTAATGG | NC_045512.2 | 3′→5′ | 16469 | 16484 |
| 716 | AACCAGTAAAGACATA | NC_045512.2 | 3′→5′ | 16774 | 16789 |
| 717 | TAACCAGTAAAGACAT | NC_045512.2 | 3′→5′ | 16775 | 16790 |
| 718 | CAAAGGTGTACTCTCC | NC_045512.2 | 3′→5′ | 16822 | 16837 |
| 719 | TCAAAGGTGTACTCTC | NC_045512.2 | 3′→5′ | 16823 | 16838 |
| 720 | TTCAAAGGTGTACTCT | NC_045512.2 | 3′→5′ | 16824 | 16839 |
| 721 | TTTCAAAGGTGTACTC | NC_045512.2 | 3′→5′ | 16825 | 16840 |
| 722 | TTTTCAAAGGTGTACT | NC_045512.2 | 3′→5′ | 16826 | 16841 |
| 723 | TTTTTCAAAGGTGTAC | NC_045512.2 | 3′→5′ | 16827 | 16842 |
| 724 | CTTTTTCAAAGGTGTA | NC_045512.2 | 3′→5′ | 16828 | 16843 |
| 725 | CCTTTTTCAAAGGTGT | NC_045512.2 | 3′→5′ | 16829 | 16844 |
| 726 | ACCTTTTTCAAAGGTG | NC_045512.2 | 3′→5′ | 16830 | 16845 |
| 727 | CACCTTTTTCAAAGGT | NC_045512.2 | 3′→5′ | 16831 | 16846 |
| 728 | TCACCTTTTTCAAAGG | NC_045512.2 | 3′→5′ | 16832 | 16847 |
| 729 | GTCACCTTTTTCAAAG | NC_045512.2 | 3′→5′ | 16833 | 16848 |
| 730 | AGTCACCTTTTTCAAA | NC_045512.2 | 3′→5′ | 16834 | 16849 |
| 731 | TAGTCACCTTTTTCAA | NC_045512.2 | 3′→5′ | 16835 | 16850 |
| 732 | ATAGTCACCTTTTTCA | NC_045512.2 | 3′→5′ | 16836 | 16851 |
| 733 | CATAGTCACCTTTTTC | NC_045512.2 | 3′→5′ | 16837 | 16852 |
| 734 | CCATAGTCACCTTTTT | NC_045512.2 | 3′→5′ | 16838 | 16853 |
| 735 | ACCATAGTCACCTTTT | NC_045512.2 | 3′→5′ | 16839 | 16854 |
| 736 | CACCATAGTCACCTTT | NC_045512.2 | 3′→5′ | 16840 | 16855 |
| 737 | TCACCATAGTCACCTT | NC_045512.2 | 3′→5′ | 16841 | 16856 |
| 738 | ATCACCATAGTCACCT | NC_045512.2 | 3′→5′ | 16842 | 16857 |
| 739 | CATCACCATAGTCACC | NC_045512.2 | 3′→5′ | 16843 | 16858 |
| 740 | GCATCACCATAGTCAC | NC_045512.2 | 3′→5′ | 16844 | 16859 |
| 741 | AGCATCACCATAGTCA | NC_045512.2 | 3′→5′ | 16845 | 16860 |
| 742 | CAGCATCACCATAGTC | NC_045512.2 | 3′→5′ | 16846 | 16861 |
| 743 | ACAGCATCACCATAGT | NC_045512.2 | 3′→5′ | 16847 | 16862 |
| 744 | AACAGCATCACCATAG | NC_045512.2 | 3′→5′ | 16848 | 16863 |
| 745 | CAACAGCATCACCATA | NC_045512.2 | 3′→5′ | 16849 | 16864 |
| 746 | ACAACAGCATCACCAT | NC_045512.2 | 3′→5′ | 16850 | 16865 |
| 747 | GCTCTTGTGGCACTAG | NC_045512.2 | 3′→5′ | 16954 | 16969 |
| 748 | TGCTCTTGTGGCACTA | NC_045512.2 | 3′→5′ | 16955 | 16970 |
| 749 | GTGCTCTTGTGGCACT | NC_045512.2 | 3′→5′ | 16956 | 16971 |
| 750 | AGTGCTCTTGTGGCAC | NC_045512.2 | 3′→5′ | 16957 | 16972 |
| 751 | TAGTGCTCTTGTGGCA | NC_045512.2 | 3′→5′ | 16958 | 16973 |
| 752 | ATAGTGCTCTTGTGGC | NC_045512.2 | 3′→5′ | 16959 | 16974 |
| 753 | CATAGTGCTCTTGTGG | NC_045512.2 | 3′→5′ | 16960 | 16975 |
| 754 | ACATAGTGCTCTTGTG | NC_045512.2 | 3′→5′ | 16961 | 16976 |
| 755 | AAAACTCATCTGAGAT | NC_045512.2 | 3′→5′ | 17008 | 17023 |
| 756 | GAAAACTCATCTGAGA | NC_045512.2 | 3′→5′ | 17009 | 17024 |
| 757 | AGAAAACTCATCTGAG | NC_045512.2 | 3′→5′ | 17010 | 17025 |
| 758 | TAGAAAACTCATCTGA | NC_045512.2 | 3′→5′ | 17011 | 17026 |
| 759 | CTAGAAAACTCATCTG | NC_045512.2 | 3′→5′ | 17012 | 17027 |
| 760 | GCTAGAAAACTCATCT | NC_045512.2 | 3′→5′ | 17013 | 17028 |
| 761 | TGCTAGAAAACTCATC | NC_045512.2 | 3′→5′ | 17014 | 17029 |
| 762 | TTGCTAGAAAACTCAT | NC_045512.2 | 3′→5′ | 17015 | 17030 |
| 763 | ATTGCTAGAAAACTCA | NC_045512.2 | 3′→5′ | 17016 | 17031 |
| 764 | CATTGCTAGAAAACTC | NC_045512.2 | 3′→5′ | 17017 | 17032 |
| 765 | ACATTGCTAGAAAACT | NC_045512.2 | 3′→5′ | 17018 | 17033 |
| 766 | AACATTGCTAGAAAAC | NC_045512.2 | 3′→5′ | 17019 | 17034 |
| 767 | CAACATTGCTAGAAAA | NC_045512.2 | 3′→5′ | 17020 | 17035 |
| 768 | GCAACATTGCTAGAAA | NC_045512.2 | 3′→5′ | 17021 | 17036 |
| 769 | TGCAACATTGCTAGAA | NC_045512.2 | 3′→5′ | 17022 | 17037 |
| 770 | TTGCAACATTGCTAGA | NC_045512.2 | 3′→5′ | 17023 | 17038 |
| 771 | TTTGCAACATTGCTAG | NC_045512.2 | 3′→5′ | 17024 | 17039 |
| 772 | ATTTGCAACATTGCTA | NC_045512.2 | 3′→5′ | 17025 | 17040 |
| 773 | AATTTGCAACATTGCT | NC_045512.2 | 3′→5′ | 17026 | 17041 |
| 774 | TAATTTGCAACATTGC | NC_045512.2 | 3′→5′ | 17027 | 17042 |
| 775 | CAGTACCAGGTGGTCC | NC_045512.2 | 3′→5′ | 17080 | 17095 |
| 776 | CCAGTACCAGGTGGTC | NC_045512.2 | 3′→5′ | 17081 | 17096 |
| 777 | ACCAGTACCAGGTGGT | NC_045512.2 | 3′→5′ | 17082 | 17097 |
| 778 | TACCAGTACCAGGTGG | NC_045512.2 | 3′→5′ | 17083 | 17098 |
| 779 | TTACCAGTACCAGGTG | NC_045512.2 | 3′→5′ | 17084 | 17099 |
| 780 | CTTACCAGTACCAGGT | NC_045512.2 | 3′→5′ | 17085 | 17100 |
| 781 | TCTTACCAGTACCAGG | NC_045512.2 | 3′→5′ | 17086 | 17101 |
| 782 | CTCTTACCAGTACCAG | NC_045512.2 | 3′→5′ | 17087 | 17102 |
| 783 | ACTCTTACCAGTACCA | NC_045512.2 | 3′→5′ | 17088 | 17103 |
| 784 | GACTCTTACCAGTACC | NC_045512.2 | 3′→5′ | 17089 | 17104 |
| 785 | TGACTCTTACCAGTAC | NC_045512.2 | 3′→5′ | 17090 | 17105 |
| 786 | ATGACTCTTACCAGTA | NC_045512.2 | 3′→5′ | 17091 | 17106 |
| 787 | AATGACTCTTACCAGT | NC_045512.2 | 3′→5′ | 17092 | 17107 |
| 788 | AAATGACTCTTACCAG | NC_045512.2 | 3′→5′ | 17093 | 17108 |
| 789 | AAAATGACTCTTACCA | NC_045512.2 | 3′→5′ | 17094 | 17109 |
| 790 | CAAAATGACTCTTACC | NC_045512.2 | 3′→5′ | 17095 | 17110 |
| 791 | GCAAAATGACTCTTAC | NC_045512.2 | 3′→5′ | 17096 | 17111 |
| 792 | ACACTATGCGAGCAGA | NC_045512.2 | 3′→5′ | 17137 | 17152 |
| 793 | TACACTATGCGAGCAG | NC_045512.2 | 3′→5′ | 17138 | 17153 |
| 794 | ATACACTATGCGAGCA | NC_045512.2 | 3′→5′ | 17139 | 17154 |
| 795 | TATACACTATGCGAGC | NC_045512.2 | 3′→5′ | 17140 | 17155 |
| 796 | GTATACACTATGCGAG | NC_045512.2 | 3′→5′ | 17141 | 17156 |
| 797 | AATTCACTTTGAATTT | NC_045512.2 | 3′→5′ | 17269 | 17284 |
| 798 | GAATTCACTTTGAATT | NC_045512.2 | 3′→5′ | 17270 | 17285 |
| 799 | TGAATTCACTTTGAAT | NC_045512.2 | 3′→5′ | 17271 | 17286 |
| 800 | TTGAATTCACTTTGAA | NC_045512.2 | 3′→5′ | 17272 | 17287 |
| 801 | GTTGAATTCACTTTGA | NC_045512.2 | 3′→5′ | 17273 | 17288 |
| 802 | TGTTGAATTCACTTTG | NC_045512.2 | 3′→5′ | 17274 | 17289 |
| 803 | CATTGACAACACTCAA | NC_045512.2 | 3′→5′ | 17386 | 17401 |
| 804 | GCATTGACAACACTCA | NC_045512.2 | 3′→5′ | 17387 | 17402 |
| 805 | ACATGTCTGGACCTAT | NC_045512.2 | 3′→5′ | 17530 | 17545 |
| 806 | AACATGTCTGGACCTA | NC_045512.2 | 3′→5′ | 17531 | 17546 |
| 807 | GAACATGTCTGGACCT | NC_045512.2 | 3′→5′ | 17532 | 17547 |
| 808 | GGAACATGTCTGGACC | NC_045512.2 | 3′→5′ | 17533 | 17548 |
| 809 | AGGAACATGTCTGGAC | NC_045512.2 | 3′→5′ | 17534 | 17549 |
| 810 | TTTCAGCAGGACAACG | NC_045512.2 | 3′→5′ | 17563 | 17578 |
| 811 | ATTTCAGCAGGACAAC | NC_045512.2 | 3′→5′ | 17564 | 17579 |
| 812 | AATTTCAGCAGGACAA | NC_045512.2 | 3′→5′ | 17565 | 17580 |
| 813 | CAATTTCAGCAGGACA | NC_045512.2 | 3′→5′ | 17566 | 17581 |
| 814 | ACAATTTCAGCAGGAC | NC_045512.2 | 3′→5′ | 17567 | 17582 |
| 815 | CAGATGAAACATCATG | NC_045512.2 | 3′→5′ | 17680 | 17695 |
| 816 | GCAGATGAAACATCAT | NC_045512.2 | 3′→5′ | 17681 | 17696 |
| 817 | TGCAGATGAAACATCA | NC_045512.2 | 3′→5′ | 17682 | 17697 |
| 818 | TTGCAGATGAAACATC | NC_045512.2 | 3′→5′ | 17683 | 17698 |
| 819 | ATTGCAGATGAAACAT | NC_045512.2 | 3′→5′ | 17684 | 17699 |
| 820 | CTTTTCTCCAAGCAGG | NC_045512.2 | 3′→5′ | 17746 | 17761 |
| 821 | GCTTTTCTCCAAGCAG | NC_045512.2 | 3′→5′ | 17747 | 17762 |
| 822 | AGCTTTTCTCCAAGCA | NC_045512.2 | 3′→5′ | 17748 | 17763 |
| 823 | CAGCTTTTCTCCAAGC | NC_045512.2 | 3′→5′ | 17749 | 17764 |
| 824 | ACAGCTTTTCTCCAAG | NC_045512.2 | 3′→5′ | 17750 | 17765 |
| 825 | ATATGACATAGTCATA | NC_045512.2 | 3′→5′ | 17857 | 17872 |
| 826 | AATATGACATAGTCAT | NC_045512.2 | 3′→5′ | 17858 | 17873 |
| 827 | GAATATGACATAGTCA | NC_045512.2 | 3′→5′ | 17859 | 17874 |
| 828 | TGAATATGACATAGTC | NC_045512.2 | 3′→5′ | 17860 | 17875 |
| 829 | GTGAATATGACATAGT | NC_045512.2 | 3′→5′ | 17861 | 17876 |
| 830 | TATCAGACATTATGCA | NC_045512.2 | 3′→5′ | 17956 | 17971 |
| 831 | CTATCAGACATTATGC | NC_045512.2 | 3′→5′ | 17957 | 17972 |
| 832 | TCTATCAGACATTATG | NC_045512.2 | 3′→5′ | 17958 | 17973 |
| 833 | CTCTATCAGACATTAT | NC_045512.2 | 3′→5′ | 17959 | 17974 |
| 834 | TCTCTATCAGACATTA | NC_045512.2 | 3′→5′ | 17960 | 17975 |
| 835 | GTGTAGGTGCCTGTGT | NC_045512.2 | 3′→5′ | 18100 | 18115 |
| 836 | TGTGTAGGTGCCTGTG | NC_045512.2 | 3′→5′ | 18101 | 18116 |
| 837 | GTGTGTAGGTGCCTGT | NC_045512.2 | 3′→5′ | 18102 | 18117 |
| 838 | GGTGTGTAGGTGCCTG | NC_045512.2 | 3′→5′ | 18103 | 18118 |
| 839 | AGGTGTGTAGGTGCCT | NC_045512.2 | 3′→5′ | 18104 | 18119 |
| 840 | GAGGTGTGTAGGTGCC | NC_045512.2 | 3′→5′ | 18105 | 18120 |
| 841 | TGAGGTGTGTAGGTGC | NC_045512.2 | 3′→5′ | 18106 | 18121 |
| 842 | CTGAGGTGTGTAGGTG | NC_045512.2 | 3′→5′ | 18107 | 18122 |
| 843 | TCATAGAGATGAGTCT | NC_045512.2 | 3′→5′ | 18196 | 18211 |
| 844 | ATCATAGAGATGAGTC | NC_045512.2 | 3′→5′ | 18197 | 18212 |
| 845 | CATCATAGAGATGAGT | NC_045512.2 | 3′→5′ | 18198 | 18213 |
| 846 | CCATCATAGAGATGAG | NC_045512.2 | 3′→5′ | 18199 | 18214 |
| 847 | CCCATCATAGAGATGA | NC_045512.2 | 3′→5′ | 18200 | 18215 |
| 848 | ACCCATCATAGAGATG | NC_045512.2 | 3′→5′ | 18201 | 18216 |
| 849 | AACCCATCATAGAGAT | NC_045512.2 | 3′→5′ | 18202 | 18217 |
| 850 | AAACCCATCATAGAGA | NC_045512.2 | 3′→5′ | 18203 | 18218 |
| 851 | GCTTAACAAAGCACTC | NC_045512.2 | 3′→5′ | 18889 | 18904 |
| 852 | CGCTTAACAAAGCACT | NC_045512.2 | 3′→5′ | 18890 | 18905 |
| 853 | CATTTTCTAAACTCTG | NC_045512.2 | 3′→5′ | 19618 | 19633 |
| 854 | ACATTTTCTAAACTCT | NC_045512.2 | 3′→5′ | 19619 | 19634 |
| 855 | CACATTTTCTAAACTC | NC_045512.2 | 3′→5′ | 19620 | 19635 |
| 856 | CCACATTTTCTAAACT | NC_045512.2 | 3′→5′ | 19621 | 19636 |
| 857 | GCCACATTTTCTAAAC | NC_045512.2 | 3′→5′ | 19622 | 19637 |
| 858 | AGCCACATTTTCTAAA | NC_045512.2 | 3′→5′ | 19623 | 19638 |
| 859 | AAGCCACATTTTCTAA | NC_045512.2 | 3′→5′ | 19624 | 19639 |
| 860 | GTCCATCAAAGTGTCC | NC_045512.2 | 3′→5′ | 19657 | 19672 |
| 861 | TGTCCATCAAAGTGTC | NC_045512.2 | 3′→5′ | 19658 | 19673 |
| 862 | TAGCCCAAAGCTCAAA | NC_045512.2 | 3′→5′ | 19783 | 19798 |
| 863 | TTAGCCCAAAGCTCAA | NC_045512.2 | 3′→5′ | 19784 | 19799 |
| 864 | CTTAGCCCAAAGCTCA | NC_045512.2 | 3′→5′ | 19785 | 19800 |
| 865 | GCTTAGCCCAAAGCTC | NC_045512.2 | 3′→5′ | 19786 | 19801 |
| 866 | CGCTTAGCCCAAAGCT | NC_045512.2 | 3′→5′ | 19787 | 19802 |
| 867 | CCAAATTATTGAGTAT | NC_045512.2 | 3′→5′ | 19831 | 19846 |
| 868 | CCCAAATTATTGAGTA | NC_045512.2 | 3′→5′ | 19832 | 19847 |
| 869 | ACCCAAATTATTGAGT | NC_045512.2 | 3′→5′ | 19833 | 19848 |
| 870 | CACCCAAATTATTGAG | NC_045512.2 | 3′→5′ | 19834 | 19849 |
| 871 | ACACCCAAATTATTGA | NC_045512.2 | 3′→5′ | 19835 | 19850 |
| 872 | TTAATGTGACTCCATT | NC_045512.2 | 3′→5′ | 20107 | 20122 |
| 873 | ATTAATGTGACTCCAT | NC_045512.2 | 3′→5′ | 20108 | 20123 |
| 874 | AATTAATGTGACTCCA | NC_045512.2 | 3′→5′ | 20109 | 20124 |
| 875 | CAATTAATGTGACTCC | NC_045512.2 | 3′→5′ | 20110 | 20125 |
| 876 | CCAATTAATGTGACTC | NC_045512.2 | 3′→5′ | 20111 | 20126 |
| 877 | TCCAATTAATGTGACT | NC_045512.2 | 3′→5′ | 20112 | 20127 |
| 878 | CTCCAATTAATGTGAC | NC_045512.2 | 3′→5′ | 20113 | 20128 |
| 879 | TCTCCAATTAATGTGA | NC_045512.2 | 3′→5′ | 20114 | 20129 |
| 880 | TTCTCCAATTAATGTG | NC_045512.2 | 3′→5′ | 20115 | 20130 |
| 881 | CGACATTCATCATTAT | NC_045512.2 | 3′→5′ | 20776 | 20791 |
| 882 | GCGACATTCATCATTA | NC_045512.2 | 3′→5′ | 20777 | 20792 |
| 883 | TGCGACATTCATCATT | NC_045512.2 | 3′→5′ | 20778 | 20793 |
| 884 | TTGCGACATTCATCAT | NC_045512.2 | 3′→5′ | 20779 | 20794 |
| 885 | TTTGCGACATTCATCA | NC_045512.2 | 3′→5′ | 20780 | 20795 |
| 886 | CAGCATTCCAAGAATG | NC_045512.2 | 3′→5′ | 21178 | 21193 |
| 887 | TCAGCATTCCAAGAAT | NC_045512.2 | 3′→5′ | 21179 | 21194 |
| 888 | CAATTAAAAATGCTTC | NC_045512.2 | 3′→5′ | 21265 | 21280 |
| 889 | CCAATTAAAAATGCTT | NC_045512.2 | 3′→5′ | 21266 | 21281 |
| 890 | TGTTGTTTTCTCTAAT | NC_045512.2 | 3′→5′ | 21502 | 21517 |
| 891 | CTGTTGTTTTCTCTAA | NC_045512.2 | 3′→5′ | 21503 | 21518 |
| 892 | TCTGTTGTTTTCTCTA | NC_045512.2 | 3′→5′ | 21504 | 21519 |
| 893 | CTCTGTTGTTTTCTCT | NC_045512.2 | 3′→5′ | 21505 | 21520 |
| 894 | ACTCTGTTGTTTTCTC | NC_045512.2 | 3′→5′ | 21506 | 21521 |
| 895 | AACTCTGTTGTTTTCT | NC_045512.2 | 3′→5′ | 21507 | 21522 |
| 896 | CAACTCTGTTGTTTTC | NC_045512.2 | 3′→5′ | 21508 | 21523 |
| 897 | ACAACTCTGTTGTTTT | NC_045512.2 | 3′→5′ | 21509 | 21524 |
| 898 | ATATTTGTGAAAAATT | NC_045512.2 | 3′→5′ | 23963 | 23978 |
| 899 | AATATTTGTGAAAAAT | NC_045512.2 | 3′→5′ | 23964 | 23979 |
| 900 | CCATTTGCATAGCAAA | NC_045512.2 | 3′→5′ | 24254 | 24269 |
| 901 | GCCATTTGCATAGCAA | NC_045512.2 | 3′→5′ | 24255 | 24270 |
| 902 | TCTCATAGAGAACATT | NC_045512.2 | 3′→5′ | 24302 | 24317 |
| 903 | TTCTCATAGAGAACAT | NC_045512.2 | 3′→5′ | 24303 | 24318 |
| 904 | GTTCTCATAGAGAACA | NC_045512.2 | 3′→5′ | 24304 | 24319 |
| 905 | GGTTCTCATAGAGAAC | NC_045512.2 | 3′→5′ | 24305 | 24320 |
| 906 | TGGTTCTCATAGAGAA | NC_045512.2 | 3′→5′ | 24306 | 24321 |
| 907 | TTGGTTCTCATAGAGA | NC_045512.2 | 3′→5′ | 24307 | 24322 |
| 908 | TTTGGTTCTCATAGAG | NC_045512.2 | 3′→5′ | 24308 | 24323 |
| 909 | TTTTGGTTCTCATAGA | NC_045512.2 | 3′→5′ | 24309 | 24324 |
| 910 | TTTTTGGTTCTCATAG | NC_045512.2 | 3′→5′ | 24310 | 24325 |
| 911 | TAAGTTGTTTAACAAG | NC_045512.2 | 3′→5′ | 24446 | 24461 |
| 912 | CTAAGTTGTTTAACAA | NC_045512.2 | 3′→5′ | 24447 | 24462 |
| 913 | GCTAAGTTGTTTAACA | NC_045512.2 | 3′→5′ | 24448 | 24463 |
| 914 | AGCTAAGTTGTTTAAC | NC_045512.2 | 3′→5′ | 24449 | 24464 |
| 915 | GAGCTAAGTTGTTTAA | NC_045512.2 | 3′→5′ | 24450 | 24465 |
| 916 | CAAGATTAGCAGAAGC | NC_045512.2 | 3′→5′ | 24620 | 24635 |
| 917 | GCAAGATTAGCAGAAG | NC_045512.2 | 3′→5′ | 24621 | 24636 |
| 918 | AGCAAGATTAGCAGAA | NC_045512.2 | 3′→5′ | 24622 | 24637 |
| 919 | CAGCAAGATTAGCAGA | NC_045512.2 | 3′→5′ | 24623 | 24638 |
| 920 | GCAGCAAGATTAGCAG | NC_045512.2 | 3′→5′ | 24624 | 24639 |
| 921 | AGCAGCAAGATTAGCA | NC_045512.2 | 3′→5′ | 24625 | 24640 |
| 922 | TAGCAGCAAGATTAGC | NC_045512.2 | 3′→5′ | 24626 | 24641 |
| 923 | GTAGCAGCAAGATTAG | NC_045512.2 | 3′→5′ | 24627 | 24642 |
| 924 | AGTAGCAGCAAGATTA | NC_045512.2 | 3′→5′ | 24628 | 24643 |
| 925 | TAGTAGCAGCAAGATT | NC_045512.2 | 3′→5′ | 24629 | 24644 |
| 926 | TTAGTAGCAGCAAGAT | NC_045512.2 | 3′→5′ | 24630 | 24645 |
| 927 | TTTAGTAGCAGCAAGA | NC_045512.2 | 3′→5′ | 24631 | 24646 |
| 928 | TTTTAGTAGCAGCAAG | NC_045512.2 | 3′→5′ | 24632 | 24647 |
| 929 | ATTTTAGTAGCAGCAA | NC_045512.2 | 3′→5′ | 24633 | 24648 |
| 930 | CATTTTAGTAGCAGCA | NC_045512.2 | 3′→5′ | 24634 | 24649 |
| 931 | ACATTTTAGTAGCAGC | NC_045512.2 | 3′→5′ | 24635 | 24650 |
| 932 | GACATTTTAGTAGCAG | NC_045512.2 | 3′→5′ | 24636 | 24651 |
| 933 | TTTTTGATTGTCCAAG | NC_045512.2 | 3′→5′ | 24662 | 24677 |
| 934 | CTTTTTGATTGTCCAA | NC_045512.2 | 3′→5′ | 24663 | 24678 |
| 935 | TCTTTTTGATTGTCCA | NC_045512.2 | 3′→5′ | 24664 | 24679 |
| 936 | CTCTTTTTGATTGTCC | NC_045512.2 | 3′→5′ | 24665 | 24680 |
| 937 | ACTCTTTTTGATTGTC | NC_045512.2 | 3′→5′ | 24666 | 24681 |
| 938 | AACTCTTTTTGATTGT | NC_045512.2 | 3′→5′ | 24667 | 24682 |
| 939 | CAACTCTTTTTGATTG | NC_045512.2 | 3′→5′ | 24668 | 24683 |
| 940 | TCAACTCTTTTTGATT | NC_045512.2 | 3′→5′ | 24669 | 24684 |
| 941 | CTGGTGATGTATGATT | NC_045512.2 | 3′→5′ | 25034 | 25049 |
| 942 | TCTGGTGATGTATGAT | NC_045512.2 | 3′→5′ | 25035 | 25050 |
| 943 | ATCTGGTGATGTATGA | NC_045512.2 | 3′→5′ | 25036 | 25051 |
| 944 | CATCTGGTGATGTATG | NC_045512.2 | 3′→5′ | 25037 | 25052 |
| 945 | ACATCTGGTGATGTAT | NC_045512.2 | 3′→5′ | 25038 | 25053 |
| 946 | AACATCTGGTGATGTA | NC_045512.2 | 3′→5′ | 25039 | 25054 |
| 947 | CAACATCTGGTGATGT | NC_045512.2 | 3′→5′ | 25040 | 25055 |
| 948 | TCAACATCTGGTGATG | NC_045512.2 | 3′→5′ | 25041 | 25056 |
| 949 | ATCAACATCTGGTGAT | NC_045512.2 | 3′→5′ | 25042 | 25057 |
| 950 | AGGCGGTCAATTTCTT | NC_045512.2 | 3′→5′ | 25104 | 25119 |
| 951 | GAGGCGGTCAATTTCT | NC_045512.2 | 3′→5′ | 25105 | 25120 |
| 952 | TGAGGCGGTCAATTTC | NC_045512.2 | 3′→5′ | 25106 | 25121 |
| 953 | TTGAGGCGGTCAATTT | NC_045512.2 | 3′→5′ | 25107 | 25122 |
| 954 | ATTGAGGCGGTCAATT | NC_045512.2 | 3′→5′ | 25108 | 25123 |
| 955 | CATTGAGGCGGTCAAT | NC_045512.2 | 3′→5′ | 25109 | 25124 |
| 956 | TCATTGAGGCGGTCAA | NC_045512.2 | 3′→5′ | 25110 | 25125 |
| 957 | CTCATTGAGGCGGTCA | NC_045512.2 | 3′→5′ | 25111 | 25126 |
| 958 | CCTCATTGAGGCGGTC | NC_045512.2 | 3′→5′ | 25112 | 25127 |
| 959 | ACCTCATTGAGGCGGT | NC_045512.2 | 3′→5′ | 25113 | 25128 |
| 960 | TGTAATGTAATTTGAC | NC_045512.2 | 3′→5′ | 25364 | 25379 |
| 961 | GTGTAATGTAATTTGA | NC_045512.2 | 3′→5′ | 25365 | 25380 |
| 962 | TGTGTAATGTAATTTG | NC_045512.2 | 3′→5′ | 25366 | 25381 |
| 963 | ATGTGTAATGTAATTT | NC_045512.2 | 3′→5′ | 25367 | 25382 |
| 964 | TATGTGTAATGTAATT | NC_045512.2 | 3′→5′ | 25368 | 25383 |
| 965 | TTATGTGTAATGTAAT | NC_045512.2 | 3′→5′ | 25369 | 25384 |
| 966 | TTTATGTGTAATGTAA | NC_045512.2 | 3′→5′ | 25370 | 25385 |
| 967 | GTTTATGTGTAATGTA | NC_045512.2 | 3′→5′ | 25371 | 25386 |
| 968 | CGTTTATGTGTAATGT | NC_045512.2 | 3′→5′ | 25372 | 25387 |
| 969 | GGGAGTGAGGCTTGTA | NC_045512.2 | 3′→5′ | 25502 | 25517 |
| 970 | AGGGAGTGAGGCTTGT | NC_045512.2 | 3′→5′ | 25503 | 25518 |
| 971 | AAGGGAGTGAGGCTTG | NC_045512.2 | 3′→5′ | 25504 | 25519 |
| 972 | AAAGGGAGTGAGGCTT | NC_045512.2 | 3′→5′ | 25505 | 25520 |
| 973 | GAAAGGGAGTGAGGCT | NC_045512.2 | 3′→5′ | 25506 | 25521 |
| 974 | CGAAAGGGAGTGAGGC | NC_045512.2 | 3′→5′ | 25507 | 25522 |
| 975 | CCGAAAGGGAGTGAGG | NC_045512.2 | 3′→5′ | 25508 | 25523 |
| 976 | TCCGAAAGGGAGTGAG | NC_045512.2 | 3′→5′ | 25509 | 25524 |
| 977 | ATCCGAAAGGGAGTGA | NC_045512.2 | 3′→5′ | 25510 | 25525 |
| 978 | CATCCGAAAGGGAGTG | NC_045512.2 | 3′→5′ | 25511 | 25526 |
| 979 | CCATCCGAAAGGGAGT | NC_045512.2 | 3′→5′ | 25512 | 25527 |
| 980 | GCCATCCGAAAGGGAG | NC_045512.2 | 3′→5′ | 25513 | 25528 |
| 981 | AGCCATCCGAAAGGGA | NC_045512.2 | 3′→5′ | 25514 | 25529 |
| 982 | AAGCCATCCGAAAGGG | NC_045512.2 | 3′→5′ | 25515 | 25530 |
| 983 | TGAAAAACAGCAAGAA | NC_045512.2 | 3′→5′ | 25547 | 25562 |
| 984 | TAGTAGTCGTCGTCGG | NC_045512.2 | 3′→5′ | 26191 | 26206 |
| 985 | CTAGTAGTCGTCGTCG | NC_045512.2 | 3′→5′ | 26192 | 26207 |
| 986 | GCTAGTAGTCGTCGTC | NC_045512.2 | 3′→5′ | 26193 | 26208 |
| 987 | CGCTAGTAGTCGTCGT | NC_045512.2 | 3′→5′ | 26194 | 26209 |
| 988 | ACGCTAGTAGTCGTCG | NC_045512.2 | 3′→5′ | 26195 | 26210 |
| 989 | CACGCTAGTAGTCGTC | NC_045512.2 | 3′→5′ | 26196 | 26211 |
| 990 | GCACGCTAGTAGTCGT | NC_045512.2 | 3′→5′ | 26197 | 26212 |
| 991 | GGCACGCTAGTAGTCG | NC_045512.2 | 3′→5′ | 26198 | 26213 |
| 992 | AGGCACGCTAGTAGTC | NC_045512.2 | 3′→5′ | 26199 | 26214 |
| 993 | AAGGCACGCTAGTAGT | NC_045512.2 | 3′→5′ | 26200 | 26215 |
| 994 | AAAGGCACGCTAGTAG | NC_045512.2 | 3′→5′ | 26201 | 26216 |
| 995 | CAAAGGCACGCTAGTA | NC_045512.2 | 3′→5′ | 26202 | 26217 |
| 996 | ACAAAGGCACGCTAGT | NC_045512.2 | 3′→5′ | 26203 | 26218 |
| 997 | TACAAAGGCACGCTAG | NC_045512.2 | 3′→5′ | 26204 | 26219 |
| 998 | TTACAAAGGCACGCTA | NC_045512.2 | 3′→5′ | 26205 | 26220 |
| 999 | CTTACAAAGGCACGCT | NC_045512.2 | 3′→5′ | 26206 | 26221 |
| 1000 | GCTTACAAAGGCACGC | NC_045512.2 | 3′→5′ | 26207 | 26222 |
| 1001 | TGCTTACAAAGGCACG | NC_045512.2 | 3′→5′ | 26208 | 26223 |
| 1002 | GTGCTTACAAAGGCAC | NC_045512.2 | 3′→5′ | 26209 | 26224 |
| 1003 | TGTGCTTACAAAGGCA | NC_045512.2 | 3′→5′ | 26210 | 26225 |
| 1004 | TTGTGCTTACAAAGGC | NC_045512.2 | 3′→5′ | 26211 | 26226 |
| 1005 | CTTGTGCTTACAAAGG | NC_045512.2 | 3′→5′ | 26212 | 26227 |
| 1006 | CATAAGTTCGTACTCA | NC_045512.2 | 3′→5′ | 26232 | 26247 |
| 1007 | ACATAAGTTCGTACTC | NC_045512.2 | 3′→5′ | 26233 | 26248 |
| 1008 | TACATAAGTTCGTACT | NC_045512.2 | 3′→5′ | 26234 | 26249 |
| 1009 | GTACATAAGTTCGTAC | NC_045512.2 | 3′→5′ | 26235 | 26250 |
| 1010 | AGTACATAAGTTCGTA | NC_045512.2 | 3′→5′ | 26236 | 26251 |
| 1011 | GAGTACATAAGTTCGT | NC_045512.2 | 3′→5′ | 26237 | 26252 |
| 1012 | TGAGTACATAAGTTCG | NC_045512.2 | 3′→5′ | 26238 | 26253 |
| 1013 | ATGAGTACATAAGTTC | NC_045512.2 | 3′→5′ | 26239 | 26254 |
| 1014 | AATGAGTACATAAGTT | NC_045512.2 | 3′→5′ | 26240 | 26255 |
| 1015 | GAATGAGTACATAAGT | NC_045512.2 | 3′→5′ | 26241 | 26256 |
| 1016 | CGAATGAGTACATAAG | NC_045512.2 | 3′→5′ | 26242 | 26257 |
| 1017 | ACGAATGAGTACATAA | NC_045512.2 | 3′→5′ | 26243 | 26258 |
| 1018 | AACGAATGAGTACATA | NC_045512.2 | 3′→5′ | 26244 | 26259 |
| 1019 | AAACGAATGAGTACAT | NC_045512.2 | 3′→5′ | 26245 | 26260 |
| 1020 | GAAACGAATGAGTACA | NC_045512.2 | 3′→5′ | 26246 | 26261 |
| 1021 | CGAAACGAATGAGTAC | NC_045512.2 | 3′→5′ | 26247 | 26262 |
| 1022 | CCGAAACGAATGAGTA | NC_045512.2 | 3′→5′ | 26248 | 26263 |
| 1023 | TCCGAAACGAATGAGT | NC_045512.2 | 3′→5′ | 26249 | 26264 |
| 1024 | TTCCGAAACGAATGAG | NC_045512.2 | 3′→5′ | 26250 | 26265 |
| 1025 | CTTCCGAAACGAATGA | NC_045512.2 | 3′→5′ | 26251 | 26266 |
| 1026 | TCTTCCGAAACGAATG | NC_045512.2 | 3′→5′ | 26252 | 26267 |
| 1027 | CTATTAACGTACCTGT | NC_045512.2 | 3′→5′ | 26269 | 26284 |
| 1028 | ACTATTAACGTACCTG | NC_045512.2 | 3′→5′ | 26270 | 26285 |
| 1029 | AACTATTAACGTACCT | NC_045512.2 | 3′→5′ | 26271 | 26286 |
| 1030 | TAACTATTAACGTACC | NC_045512.2 | 3′→5′ | 26272 | 26287 |
| 1031 | TTAACTATTAACGTAC | NC_045512.2 | 3′→5′ | 26273 | 26288 |
| 1032 | ATTAACTATTAACGTA | NC_045512.2 | 3′→5′ | 26274 | 26289 |
| 1033 | TATTAACTATTAACGT | NC_045512.2 | 3′→5′ | 26275 | 26290 |
| 1034 | CTATTAACTATTAACG | NC_045512.2 | 3′→5′ | 26276 | 26291 |
| 1035 | GCTATTAACTATTAAC | NC_045512.2 | 3′→5′ | 26277 | 26292 |
| 1036 | CGCTATTAACTATTAA | NC_045512.2 | 3′→5′ | 26278 | 26293 |
| 1037 | ACGCTATTAACTATTA | NC_045512.2 | 3′→5′ | 26279 | 26294 |
| 1038 | TACGCTATTAACTATT | NC_045512.2 | 3′→5′ | 26280 | 26295 |
| 1039 | GTACGCTATTAACTAT | NC_045512.2 | 3′→5′ | 26281 | 26296 |
| 1040 | AGTACGCTATTAACTA | NC_045512.2 | 3′→5′ | 26282 | 26297 |
| 1041 | AAGTACGCTATTAACT | NC_045512.2 | 3′→5′ | 26283 | 26298 |
| 1042 | GAAGTACGCTATTAAC | NC_045512.2 | 3′→5′ | 26284 | 26299 |
| 1043 | AGAAGTACGCTATTAA | NC_045512.2 | 3′→5′ | 26285 | 26300 |
| 1044 | AAGAAGTACGCTATTA | NC_045512.2 | 3′→5′ | 26286 | 26301 |
| 1045 | AAAGAAGTACGCTATT | NC_045512.2 | 3′→5′ | 26287 | 26302 |
| 1046 | AAAAGAAGTACGCTAT | NC_045512.2 | 3′→5′ | 26288 | 26303 |
| 1047 | AAAAAGAAGTACGCTA | NC_045512.2 | 3′→5′ | 26289 | 26304 |
| 1048 | GAAAAAGAAGTACGCT | NC_045512.2 | 3′→5′ | 26290 | 26305 |
| 1049 | AGAAAAAGAAGTACGC | NC_045512.2 | 3′→5′ | 26291 | 26306 |
| 1050 | AAGAAAAAGAAGTACG | NC_045512.2 | 3′→5′ | 26292 | 26307 |
| 1051 | CAAGAAAAAGAAGTAC | NC_045512.2 | 3′→5′ | 26293 | 26308 |
| 1052 | GCAAGAAAAAGAAGTA | NC_045512.2 | 3′→5′ | 26294 | 26309 |
| 1053 | AGCAAGAAAAAGAAGT | NC_045512.2 | 3′→5′ | 26295 | 26310 |
| 1054 | AAGCAAGAAAAAGAAG | NC_045512.2 | 3′→5′ | 26296 | 26311 |
| 1055 | AAAGCAAGAAAAAGAA | NC_045512.2 | 3′→5′ | 26297 | 26312 |
| 1056 | GAAAGCAAGAAAAAGA | NC_045512.2 | 3′→5′ | 26298 | 26313 |
| 1057 | CGAAAGCAAGAAAAAG | NC_045512.2 | 3′→5′ | 26299 | 26314 |
| 1058 | ACGAAAGCAAGAAAAA | NC_045512.2 | 3′→5′ | 26300 | 26315 |
| 1059 | CACGAAAGCAAGAAAA | NC_045512.2 | 3′→5′ | 26301 | 26316 |
| 1060 | CCACGAAAGCAAGAAA | NC_045512.2 | 3′→5′ | 26302 | 26317 |
| 1061 | ACCACGAAAGCAAGAA | NC_045512.2 | 3′→5′ | 26303 | 26318 |
| 1062 | TACCACGAAAGCAAGA | NC_045512.2 | 3′→5′ | 26304 | 26319 |
| 1063 | ATACCACGAAAGCAAG | NC_045512.2 | 3′→5′ | 26305 | 26320 |
| 1064 | AATACCACGAAAGCAA | NC_045512.2 | 3′→5′ | 26306 | 26321 |
| 1065 | GAATACCACGAAAGCA | NC_045512.2 | 3′→5′ | 26307 | 26322 |
| 1066 | AGAATACCACGAAAGC | NC_045512.2 | 3′→5′ | 26308 | 26323 |
| 1067 | AAGAATACCACGAAAG | NC_045512.2 | 3′→5′ | 26309 | 26324 |
| 1068 | CAAGAATACCACGAAA | NC_045512.2 | 3′→5′ | 26310 | 26325 |
| 1069 | GCAAGAATACCACGAA | NC_045512.2 | 3′→5′ | 26311 | 26326 |
| 1070 | AGCAAGAATACCACGA | NC_045512.2 | 3′→5′ | 26312 | 26327 |
| 1071 | TAGCAAGAATACCACG | NC_045512.2 | 3′→5′ | 26313 | 26328 |
| 1072 | CTAGCAAGAATACCAC | NC_045512.2 | 3′→5′ | 26314 | 26329 |
| 1073 | ACTAGCAAGAATACCA | NC_045512.2 | 3′→5′ | 26315 | 26330 |
| 1074 | TAAGGATGGCTAGTGT | NC_045512.2 | 3′→5′ | 26332 | 26347 |
| 1075 | GTAAGGATGGCTAGTG | NC_045512.2 | 3′→5′ | 26333 | 26348 |
| 1076 | AGTAAGGATGGCTAGT | NC_045512.2 | 3′→5′ | 26334 | 26349 |
| 1077 | CAGTAAGGATGGCTAG | NC_045512.2 | 3′→5′ | 26335 | 26350 |
| 1078 | GCAGTAAGGATGGCTA | NC_045512.2 | 3′→5′ | 26336 | 26351 |
| 1079 | CGCAGTAAGGATGGCT | NC_045512.2 | 3′→5′ | 26337 | 26352 |
| 1080 | GCGCAGTAAGGATGGC | NC_045512.2 | 3′→5′ | 26338 | 26353 |
| 1081 | AGCGCAGTAAGGATGG | NC_045512.2 | 3′→5′ | 26339 | 26354 |
| 1082 | AAGCGCAGTAAGGATG | NC_045512.2 | 3′→5′ | 26340 | 26355 |
| 1083 | GAAGCGCAGTAAGGAT | NC_045512.2 | 3′→5′ | 26341 | 26356 |
| 1084 | CGAAGCGCAGTAAGGA | NC_045512.2 | 3′→5′ | 26342 | 26357 |
| 1085 | TCGAAGCGCAGTAAGG | NC_045512.2 | 3′→5′ | 26343 | 26358 |
| 1086 | ATCGAAGCGCAGTAAG | NC_045512.2 | 3′→5′ | 26344 | 26359 |
| 1087 | AATCGAAGCGCAGTAA | NC_045512.2 | 3′→5′ | 26345 | 26360 |
| 1088 | CAATCGAAGCGCAGTA | NC_045512.2 | 3′→5′ | 26346 | 26361 |
| 1089 | ACAATCGAAGCGCAGT | NC_045512.2 | 3′→5′ | 26347 | 26362 |
| 1090 | CACAATCGAAGCGCAG | NC_045512.2 | 3′→5′ | 26348 | 26363 |
| 1091 | ACACAATCGAAGCGCA | NC_045512.2 | 3′→5′ | 26349 | 26364 |
| 1092 | CACACAATCGAAGCGC | NC_045512.2 | 3′→5′ | 26350 | 26365 |
| 1093 | GCACACAATCGAAGCG | NC_045512.2 | 3′→5′ | 26351 | 26366 |
| 1094 | CGCACACAATCGAAGC | NC_045512.2 | 3′→5′ | 26352 | 26367 |
| 1095 | ACGCACACAATCGAAG | NC_045512.2 | 3′→5′ | 26353 | 26368 |
| 1096 | TACGCACACAATCGAA | NC_045512.2 | 3′→5′ | 26354 | 26369 |
| 1097 | GTACGCACACAATCGA | NC_045512.2 | 3′→5′ | 26355 | 26370 |
| 1098 | AGTACGCACACAATCG | NC_045512.2 | 3′→5′ | 26356 | 26371 |
| 1099 | CAGTACGCACACAATC | NC_045512.2 | 3′→5′ | 26357 | 26372 |
| 1100 | GCAGTACGCACACAAT | NC_045512.2 | 3′→5′ | 26358 | 26373 |
| 1101 | AGCAGTACGCACACAA | NC_045512.2 | 3′→5′ | 26359 | 26374 |
| 1102 | CAGCAGTACGCACACA | NC_045512.2 | 3′→5′ | 26360 | 26375 |
| 1103 | GCAGCAGTACGCACAC | NC_045512.2 | 3′→5′ | 26361 | 26376 |
| 1104 | TGCAGCAGTACGCACA | NC_045512.2 | 3′→5′ | 26362 | 26377 |
| 1105 | TTGCAGCAGTACGCAC | NC_045512.2 | 3′→5′ | 26363 | 26378 |
| 1106 | ATTGCAGCAGTACGCA | NC_045512.2 | 3′→5′ | 26364 | 26379 |
| 1107 | TATTGCAGCAGTACGC | NC_045512.2 | 3′→5′ | 26365 | 26380 |
| 1108 | ATATTGCAGCAGTACG | NC_045512.2 | 3′→5′ | 26366 | 26381 |
| 1109 | AATATTGCAGCAGTAC | NC_045512.2 | 3′→5′ | 26367 | 26382 |
| 1110 | CAATATTGCAGCAGTA | NC_045512.2 | 3′→5′ | 26368 | 26383 |
| 1111 | ACAATATTGCAGCAGT | NC_045512.2 | 3′→5′ | 26369 | 26384 |
| 1112 | AACAATATTGCAGCAG | NC_045512.2 | 3′→5′ | 26370 | 26385 |
| 1113 | TAACAATATTGCAGCA | NC_045512.2 | 3′→5′ | 26371 | 26386 |
| 1114 | TTAACAATATTGCAGC | NC_045512.2 | 3′→5′ | 26372 | 26387 |
| 1115 | GTTAACAATATTGCAG | NC_045512.2 | 3′→5′ | 26373 | 26388 |
| 1116 | CGTTAACAATATTGCA | NC_045512.2 | 3′→5′ | 26374 | 26389 |
| 1117 | ACGTTAACAATATTGC | NC_045512.2 | 3′→5′ | 26375 | 26390 |
| 1118 | CACGTTAACAATATTG | NC_045512.2 | 3′→5′ | 26376 | 26391 |
| 1119 | TCACGTTAACAATATT | NC_045512.2 | 3′→5′ | 26377 | 26392 |
| 1120 | CTCACGTTAACAATAT | NC_045512.2 | 3′→5′ | 26378 | 26393 |
| 1121 | ACTCACGTTAACAATA | NC_045512.2 | 3′→5′ | 26379 | 26394 |
| 1122 | TCAGATTTTTAACACG | NC_045512.2 | 3′→5′ | 26425 | 26440 |
| 1123 | TTCAGATTTTTAACAC | NC_045512.2 | 3′→5′ | 26426 | 26441 |
| 1124 | AGAAGATCAGGAACTC | NC_045512.2 | 3′→5′ | 26450 | 26465 |
| 1125 | CAGAAGATCAGGAACT | NC_045512.2 | 3′→5′ | 26451 | 26466 |
| 1126 | CCAGAAGATCAGGAAC | NC_045512.2 | 3′→5′ | 26452 | 26467 |
| 1127 | ACCAGAAGATCAGGAA | NC_045512.2 | 3′→5′ | 26453 | 26468 |
| 1128 | GACCAGAAGATCAGGA | NC_045512.2 | 3′→5′ | 26454 | 26469 |
| 1129 | AGACCAGAAGATCAGG | NC_045512.2 | 3′→5′ | 26455 | 26470 |
| 1130 | TAGACCAGAAGATCAG | NC_045512.2 | 3′→5′ | 26456 | 26471 |
| 1131 | TTAGACCAGAAGATCA | NC_045512.2 | 3′→5′ | 26457 | 26472 |
| 1132 | TTTAGACCAGAAGATC | NC_045512.2 | 3′→5′ | 26458 | 26473 |
| 1133 | GTTTAGACCAGAAGAT | NC_045512.2 | 3′→5′ | 26459 | 26474 |
| 1134 | CGTTTAGACCAGAAGA | NC_045512.2 | 3′→5′ | 26460 | 26475 |
| 1135 | TCGTTTAGACCAGAAG | NC_045512.2 | 3′→5′ | 26461 | 26476 |
| 1136 | TTCGTTTAGACCAGAA | NC_045512.2 | 3′→5′ | 26462 | 26477 |
| 1137 | GTTCGTTTAGACCAGA | NC_045512.2 | 3′→5′ | 26463 | 26478 |
| 1138 | AGTTCGTTTAGACCAG | NC_045512.2 | 3′→5′ | 26464 | 26479 |
| 1139 | TAGTTCGTTTAGACCA | NC_045512.2 | 3′→5′ | 26465 | 26480 |
| 1140 | TTAGTTCGTTTAGACC | NC_045512.2 | 3′→5′ | 26466 | 26481 |
| 1141 | AAAGTTCCAAACAGAA | NC_045512.2 | 3′→5′ | 26497 | 26512 |
| 1142 | TAAAGTTCCAAACAGA | NC_045512.2 | 3′→5′ | 26498 | 26513 |
| 1143 | TTAAAGTTCCAAACAG | NC_045512.2 | 3′→5′ | 26499 | 26514 |
| 1144 | CTAGGTTCCATTGTTC | NC_045512.2 | 3′→5′ | 26574 | 26589 |
| 1145 | ACTAGGTTCCATTGTT | NC_045512.2 | 3′→5′ | 26575 | 26590 |
| 1146 | TACTAGGTTCCATTGT | NC_045512.2 | 3′→5′ | 26576 | 26591 |
| 1147 | TTACTAGGTTCCATTG | NC_045512.2 | 3′→5′ | 26577 | 26592 |
| 1148 | ATTACTAGGTTCCATT | NC_045512.2 | 3′→5′ | 26578 | 26593 |
| 1149 | TATTACTAGGTTCCAT | NC_045512.2 | 3′→5′ | 26579 | 26594 |
| 1150 | CTATTACTAGGTTCCA | NC_045512.2 | 3′→5′ | 26580 | 26595 |
| 1151 | CCTATTACTAGGTTCC | NC_045512.2 | 3′→5′ | 26581 | 26596 |
| 1152 | ACCTATTACTAGGTTC | NC_045512.2 | 3′→5′ | 26582 | 26597 |
| 1153 | AACCTATTACTAGGTT | NC_045512.2 | 3′→5′ | 26583 | 26598 |
| 1154 | AAACCTATTACTAGGT | NC_045512.2 | 3′→5′ | 26584 | 26599 |
| 1155 | GAAACCTATTACTAGG | NC_045512.2 | 3′→5′ | 26585 | 26600 |
| 1156 | CAGCAGCAAGCACAAA | NC_045512.2 | 3′→5′ | 26715 | 26730 |
| 1157 | ACAGCAGCAAGCACAA | NC_045512.2 | 3′→5′ | 26716 | 26731 |
| 1158 | GCCACATCAAGCCTAC | NC_045512.2 | 3′→5′ | 26784 | 26799 |
| 1159 | AGCCACATCAAGCCTA | NC_045512.2 | 3′→5′ | 26785 | 26800 |
| 1160 | GCGTTCGTGATGTAGC | NC_045512.2 | 3′→5′ | 27033 | 27048 |
| 1161 | AGCGTTCGTGATGTAG | NC_045512.2 | 3′→5′ | 27034 | 27049 |
| 1162 | AAGCGTTCGTGATGTA | NC_045512.2 | 3′→5′ | 27035 | 27050 |
| 1163 | AAAGCGTTCGTGATGT | NC_045512.2 | 3′→5′ | 27036 | 27051 |
| 1164 | GAAAGCGTTCGTGATG | NC_045512.2 | 3′→5′ | 27037 | 27052 |
| 1165 | AGAAAGCGTTCGTGAT | NC_045512.2 | 3′→5′ | 27038 | 27053 |
| 1166 | AAGAAAGCGTTCGTGA | NC_045512.2 | 3′→5′ | 27039 | 27054 |
| 1167 | TAAGAAAGCGTTCGTG | NC_045512.2 | 3′→5′ | 27040 | 27055 |
| 1168 | ATAAGAAAGCGTTCGT | NC_045512.2 | 3′→5′ | 27041 | 27056 |
| 1169 | AATAAGAAAGCGTTCG | NC_045512.2 | 3′→5′ | 27042 | 27057 |
| 1170 | TAATAAGAAAGCGTTC | NC_045512.2 | 3′→5′ | 27043 | 27058 |
| 1171 | GTAATAAGAAAGCGTT | NC_045512.2 | 3′→5′ | 27044 | 27059 |
| 1172 | TGTAATAAGAAAGCGT | NC_045512.2 | 3′→5′ | 27045 | 27060 |
| 1173 | TTGTAATAAGAAAGCG | NC_045512.2 | 3′→5′ | 27046 | 27061 |
| 1174 | TTTGTAATAAGAAAGC | NC_045512.2 | 3′→5′ | 27047 | 27062 |
| 1175 | ATTTGTAATAAGAAAG | NC_045512.2 | 3′→5′ | 27048 | 27063 |
| 1176 | AATTTGTAATAAGAAA | NC_045512.2 | 3′→5′ | 27049 | 27064 |
| 1177 | ATGCAGCAAAACCTGA | NC_045512.2 | 3′→5′ | 27093 | 27108 |
| 1178 | TATGCAGCAAAACCTG | NC_045512.2 | 3′→5′ | 27094 | 27109 |
| 1179 | GTATGCAGCAAAACCT | NC_045512.2 | 3′→5′ | 27095 | 27110 |
| 1180 | TGTATGCAGCAAAACC | NC_045512.2 | 3′→5′ | 27096 | 27111 |
| 1181 | GCAAAGCAATATTGTC | NC_045512.2 | 3′→5′ | 27165 | 27180 |
| 1182 | AGCAAAGCAATATTGT | NC_045512.2 | 3′→5′ | 27166 | 27181 |
| 1183 | TTGTCACTTACTGTAC | NC_045512.2 | 3′→5′ | 27183 | 27198 |
| 1184 | GTTGTCACTTACTGTA | NC_045512.2 | 3′→5′ | 27184 | 27199 |
| 1185 | TGTTGTCACTTACTGT | NC_045512.2 | 3′→5′ | 27185 | 27200 |
| 1186 | CTGTTGTCACTTACTG | NC_045512.2 | 3′→5′ | 27186 | 27201 |
| 1187 | TCTGTTGTCACTTACT | NC_045512.2 | 3′→5′ | 27187 | 27202 |
| 1188 | ATCTGTTGTCACTTAC | NC_045512.2 | 3′→5′ | 27188 | 27203 |
| 1189 | CATCTGTTGTCACTTA | NC_045512.2 | 3′→5′ | 27189 | 27204 |
| 1190 | ACATCTGTTGTCACTT | NC_045512.2 | 3′→5′ | 27190 | 27205 |
| 1191 | AACATCTGTTGTCACT | NC_045512.2 | 3′→5′ | 27191 | 27206 |
| 1192 | AAACATCTGTTGTCAC | NC_045512.2 | 3′→5′ | 27192 | 27207 |
| 1193 | GAAACATCTGTTGTCA | NC_045512.2 | 3′→5′ | 27193 | 27208 |
| 1194 | TGAAACATCTGTTGTC | NC_045512.2 | 3′→5′ | 27194 | 27209 |
| 1195 | ATGAAACATCTGTTGT | NC_045512.2 | 3′→5′ | 27195 | 27210 |
| 1196 | GATGAAACATCTGTTG | NC_045512.2 | 3′→5′ | 27196 | 27211 |
| 1197 | AGATGAAACATCTGTT | NC_045512.2 | 3′→5′ | 27197 | 27212 |
| 1198 | TCATGTTCGTTTAATC | NC_045512.2 | 3′→5′ | 27382 | 27397 |
| 1199 | TTCATGTTCGTTTAAT | NC_045512.2 | 3′→5′ | 27383 | 27398 |
| 1200 | TTTCATGTTCGTTTAA | NC_045512.2 | 3′→5′ | 27384 | 27399 |
| 1201 | TTTTCATGTTCGTTTA | NC_045512.2 | 3′→5′ | 27385 | 27400 |
| 1202 | ATTTTCATGTTCGTTT | NC_045512.2 | 3′→5′ | 27386 | 27401 |
| 1203 | AATTTTCATGTTCGTT | NC_045512.2 | 3′→5′ | 27387 | 27402 |
| 1204 | TAATTTTCATGTTCGT | NC_045512.2 | 3′→5′ | 27388 | 27403 |
| 1205 | ATAATTTTCATGTTCG | NC_045512.2 | 3′→5′ | 27389 | 27404 |
| 1206 | AATAATTTTCATGTTC | NC_045512.2 | 3′→5′ | 27390 | 27405 |
| 1207 | GAATAATTTTCATGTT | NC_045512.2 | 3′→5′ | 27391 | 27406 |
| 1208 | AGAATAATTTTCATGT | NC_045512.2 | 3′→5′ | 27392 | 27407 |
| 1209 | TACCTCTAACACACTC | NC_045512.2 | 3′→5′ | 27457 | 27472 |
| 1210 | GTACCTCTAACACACT | NC_045512.2 | 3′→5′ | 27458 | 27473 |
| 1211 | GTGAATTGCCCTCGTA | NC_045512.2 | 3′→5′ | 27511 | 27526 |
| 1212 | GGTGAATTGCCCTCGT | NC_045512.2 | 3′→5′ | 27512 | 27527 |
| 1213 | TGGTGAATTGCCCTCG | NC_045512.2 | 3′→5′ | 27513 | 27528 |
| 1214 | ATGGTGAATTGCCCTC | NC_045512.2 | 3′→5′ | 27514 | 27529 |
| 1215 | AATGGTGAATTGCCCT | NC_045512.2 | 3′→5′ | 27515 | 27530 |
| 1216 | AAATGGTGAATTGCCC | NC_045512.2 | 3′→5′ | 27516 | 27531 |
| 1217 | GAAATGGTGAATTGCC | NC_045512.2 | 3′→5′ | 27517 | 27532 |
| 1218 | TGAAATGGTGAATTGC | NC_045512.2 | 3′→5′ | 27518 | 27533 |
| 1219 | CCTCTTGTCTGATGAA | NC_045512.2 | 3′→5′ | 27652 | 27667 |
| 1220 | TCCTCTTGTCTGATGA | NC_045512.2 | 3′→5′ | 27653 | 27668 |
| 1221 | AATAGAAGTCAATTAA | NC_045512.2 | 3′→5′ | 27771 | 27786 |
| 1222 | AAATAGAAGTCAATTA | NC_045512.2 | 3′→5′ | 27772 | 27787 |
| 1223 | CAAATAGAAGTCAATT | NC_045512.2 | 3′→5′ | 27773 | 27788 |
| 1224 | ACAAATAGAAGTCAAT | NC_045512.2 | 3′→5′ | 27774 | 27789 |
| 1225 | CACAAATAGAAGTCAA | NC_045512.2 | 3′→5′ | 27775 | 27790 |
| 1226 | GCACAAATAGAAGTCA | NC_045512.2 | 3′→5′ | 27776 | 27791 |
| 1227 | AGCACAAATAGAAGTC | NC_045512.2 | 3′→5′ | 27777 | 27792 |
| 1228 | AAGCACAAATAGAAGT | NC_045512.2 | 3′→5′ | 27778 | 27793 |
| 1229 | AAAGCACAAATAGAAG | NC_045512.2 | 3′→5′ | 27779 | 27794 |
| 1230 | AAAAGCACAAATAGAA | NC_045512.2 | 3′→5′ | 27780 | 27795 |
| 1231 | AAAAAGCACAAATAGA | NC_045512.2 | 3′→5′ | 27781 | 27796 |
| 1232 | TAAAAAGCACAAATAG | NC_045512.2 | 3′→5′ | 27782 | 27797 |
| 1233 | CTAAAAAGCACAAATA | NC_045512.2 | 3′→5′ | 27783 | 27798 |
| 1234 | GCTAAAAAGCACAAAT | NC_045512.2 | 3′→5′ | 27784 | 27799 |
| 1235 | GGCTAAAAAGCACAAA | NC_045512.2 | 3′→5′ | 27785 | 27800 |
| 1236 | AGGCTAAAAAGCACAA | NC_045512.2 | 3′→5′ | 27786 | 27801 |
| 1237 | AAGGCTAAAAAGCACA | NC_045512.2 | 3′→5′ | 27787 | 27802 |
| 1238 | AAAGGCTAAAAAGCAC | NC_045512.2 | 3′→5′ | 27788 | 27803 |
| 1239 | GAAAGGCTAAAAAGCA | NC_045512.2 | 3′→5′ | 27789 | 27804 |
| 1240 | AGAAAGGCTAAAAAGC | NC_045512.2 | 3′→5′ | 27790 | 27805 |
| 1241 | CAGAAAGGCTAAAAAG | NC_045512.2 | 3′→5′ | 27791 | 27806 |
| 1242 | GCAGAAAGGCTAAAAA | NC_045512.2 | 3′→5′ | 27792 | 27807 |
| 1243 | AGCAGAAAGGCTAAAA | NC_045512.2 | 3′→5′ | 27793 | 27808 |
| 1244 | TAGCAGAAAGGCTAAA | NC_045512.2 | 3′→5′ | 27794 | 27809 |
| 1245 | ATAGCAGAAAGGCTAA | NC_045512.2 | 3′→5′ | 27795 | 27810 |
| 1246 | AATAGCAGAAAGGCTA | NC_045512.2 | 3′→5′ | 27796 | 27811 |
| 1247 | GAATAGCAGAAAGGCT | NC_045512.2 | 3′→5′ | 27797 | 27812 |
| 1248 | GGAATAGCAGAAAGGC | NC_045512.2 | 3′→5′ | 27798 | 27813 |
| 1249 | AGGAATAGCAGAAAGG | NC_045512.2 | 3′→5′ | 27799 | 27814 |
| 1250 | AAGGAATAGCAGAAAG | NC_045512.2 | 3′→5′ | 27800 | 27815 |
| 1251 | CAAGGAATAGCAGAAA | NC_045512.2 | 3′→5′ | 27801 | 27816 |
| 1252 | ACAAGGAATAGCAGAA | NC_045512.2 | 3′→5′ | 27802 | 27817 |
| 1253 | AACAAGGAATAGCAGA | NC_045512.2 | 3′→5′ | 27803 | 27818 |
| 1254 | TTTCATGTTCGTTTAG | NC_045512.2 | 3′→5′ | 27884 | 27899 |
| 1255 | ATTATCAGACATTTTA | NC_045512.2 | 3′→5′ | 28270 | 28285 |
| 1256 | CATTATCAGACATTTT | NC_045512.2 | 3′→5′ | 28271 | 28286 |
| 1257 | CCATTATCAGACATTT | NC_045512.2 | 3′→5′ | 28272 | 28287 |
| 1258 | TCCATTATCAGACATT | NC_045512.2 | 3′→5′ | 28273 | 28288 |
| 1259 | GTCCATTATCAGACAT | NC_045512.2 | 3′→5′ | 28274 | 28289 |
| 1260 | GGTCCATTATCAGACA | NC_045512.2 | 3′→5′ | 28275 | 28290 |
| 1261 | GGGTCCATTATCAGAC | NC_045512.2 | 3′→5′ | 28276 | 28291 |
| 1262 | GGGGTCCATTATCAGA | NC_045512.2 | 3′→5′ | 28277 | 28292 |
| 1263 | TGGGGTCCATTATCAG | NC_045512.2 | 3′→5′ | 28278 | 28293 |
| 1264 | TTGGGGTCCATTATCA | NC_045512.2 | 3′→5′ | 28279 | 28294 |
| 1265 | TTTGGGGTCCATTATC | NC_045512.2 | 3′→5′ | 28280 | 28295 |
| 1266 | TTTTGGGGTCCATTAT | NC_045512.2 | 3′→5′ | 28281 | 28296 |
| 1267 | TGGGTAAACCTTGGGG | NC_045512.2 | 3′→5′ | 28397 | 28412 |
| 1268 | TTGGGTAAACCTTGGG | NC_045512.2 | 3′→5′ | 28398 | 28413 |
| 1269 | ATTGGGTAAACCTTGG | NC_045512.2 | 3′→5′ | 28399 | 28414 |
| 1270 | TATTGGGTAAACCTTG | NC_045512.2 | 3′→5′ | 28400 | 28415 |
| 1271 | TTATTGGGTAAACCTT | NC_045512.2 | 3′→5′ | 28401 | 28416 |
| 1272 | ATTATTGGGTAAACCT | NC_045512.2 | 3′→5′ | 28402 | 28417 |
| 1273 | TATTATTGGGTAAACC | NC_045512.2 | 3′→5′ | 28403 | 28418 |
| 1274 | GTATTATTGGGTAAAC | NC_045512.2 | 3′→5′ | 28404 | 28419 |
| 1275 | AGTATTATTGGGTAAA | NC_045512.2 | 3′→5′ | 28405 | 28420 |
| 1276 | CAGTATTATTGGGTAA | NC_045512.2 | 3′→5′ | 28406 | 28421 |
| 1277 | GCAGTATTATTGGGTA | NC_045512.2 | 3′→5′ | 28407 | 28422 |
| 1278 | CGCAGTATTATTGGGT | NC_045512.2 | 3′→5′ | 28408 | 28423 |
| 1279 | ACGCAGTATTATTGGG | NC_045512.2 | 3′→5′ | 28409 | 28424 |
| 1280 | GACGCAGTATTATTGG | NC_045512.2 | 3′→5′ | 28410 | 28425 |
| 1281 | AGACGCAGTATTATTG | NC_045512.2 | 3′→5′ | 28411 | 28426 |
| 1282 | AAGACGCAGTATTATT | NC_045512.2 | 3′→5′ | 28412 | 28427 |
| 1283 | CAAGACGCAGTATTAT | NC_045512.2 | 3′→5′ | 28413 | 28428 |
| 1284 | CCAAGACGCAGTATTA | NC_045512.2 | 3′→5′ | 28414 | 28429 |
| 1285 | ACCAAGACGCAGTATT | NC_045512.2 | 3′→5′ | 28415 | 28430 |
| 1286 | AACCAAGACGCAGTAT | NC_045512.2 | 3′→5′ | 28416 | 28431 |
| 1287 | GAACCAAGACGCAGTA | NC_045512.2 | 3′→5′ | 28417 | 28432 |
| 1288 | TGAACCAAGACGCAGT | NC_045512.2 | 3′→5′ | 28418 | 28433 |
| 1289 | GTGAACCAAGACGCAG | NC_045512.2 | 3′→5′ | 28419 | 28434 |
| 1290 | GCCAATTTGGTCATCT | NC_045512.2 | 3′→5′ | 28513 | 28528 |
| 1291 | AGCCAATTTGGTCATC | NC_045512.2 | 3′→5′ | 28514 | 28529 |
| 1292 | TAGCCAATTTGGTCAT | NC_045512.2 | 3′→5′ | 28515 | 28530 |
| 1293 | GTAGCCAATTTGGTCA | NC_045512.2 | 3′→5′ | 28516 | 28531 |
| 1294 | AGTAGCCAATTTGGTC | NC_045512.2 | 3′→5′ | 28517 | 28532 |
| 1295 | TAGTAGCCAATTTGGT | NC_045512.2 | 3′→5′ | 28518 | 28533 |
| 1296 | GTAGTAGCCAATTTGG | NC_045512.2 | 3′→5′ | 28519 | 28534 |
| 1297 | GGTAGTAGCCAATTTG | NC_045512.2 | 3′→5′ | 28520 | 28535 |
| 1298 | CGGTAGTAGCCAATTT | NC_045512.2 | 3′→5′ | 28521 | 28536 |
| 1299 | TCGGTAGTAGCCAATT | NC_045512.2 | 3′→5′ | 28522 | 28537 |
| 1300 | TTCGGTAGTAGCCAAT | NC_045512.2 | 3′→5′ | 28523 | 28538 |
| 1301 | CTTCGGTAGTAGCCAA | NC_045512.2 | 3′→5′ | 28524 | 28539 |
| 1302 | TCTTCGGTAGTAGCCA | NC_045512.2 | 3′→5′ | 28525 | 28540 |
| 1303 | CTCTTCGGTAGTAGCC | NC_045512.2 | 3′→5′ | 28526 | 28541 |
| 1304 | GCTCTTCGGTAGTAGC | NC_045512.2 | 3′→5′ | 28527 | 28542 |
| 1305 | AGCTCTTCGGTAGTAG | NC_045512.2 | 3′→5′ | 28528 | 28543 |
| 1306 | TAGCTCTTCGGTAGTA | NC_045512.2 | 3′→5′ | 28529 | 28544 |
| 1307 | GTAGCTCTTCGGTAGT | NC_045512.2 | 3′→5′ | 28530 | 28545 |
| 1308 | GGTAGCTCTTCGGTAG | NC_045512.2 | 3′→5′ | 28531 | 28546 |
| 1309 | CCGTCACCACCACGAA | NC_045512.2 | 3′→5′ | 28554 | 28569 |
| 1310 | AGGCTCCCTCAGTTGC | NC_045512.2 | 3′→5′ | 28673 | 28688 |
| 1311 | AAGGCTCCCTCAGTTG | NC_045512.2 | 3′→5′ | 28674 | 28689 |
| 1312 | CAAGGCTCCCTCAGTT | NC_045512.2 | 3′→5′ | 28675 | 28690 |
| 1313 | TCAAGGCTCCCTCAGT | NC_045512.2 | 3′→5′ | 28676 | 28691 |
| 1314 | TTCAAGGCTCCCTCAG | NC_045512.2 | 3′→5′ | 28677 | 28692 |
| 1315 | TGCGGGTGCCAATGTG | NC_045512.2 | 3′→5′ | 28706 | 28721 |
| 1316 | TTGCGGGTGCCAATGT | NC_045512.2 | 3′→5′ | 28707 | 28722 |
| 1317 | ATTGCGGGTGCCAATG | NC_045512.2 | 3′→5′ | 28708 | 28723 |
| 1318 | GATTGCGGGTGCCAAT | NC_045512.2 | 3′→5′ | 28709 | 28724 |
| 1319 | GGATTGCGGGTGCCAA | NC_045512.2 | 3′→5′ | 28710 | 28725 |
| 1320 | AGGATTGCGGGTGCCA | NC_045512.2 | 3′→5′ | 28711 | 28726 |
| 1321 | AGGAAGTTGTAGCACG | NC_045512.2 | 3′→5′ | 28744 | 28759 |
| 1322 | GAGGAAGTTGTAGCAC | NC_045512.2 | 3′→5′ | 28745 | 28760 |
| 1323 | TGAGGAAGTTGTAGCA | NC_045512.2 | 3′→5′ | 28746 | 28761 |
| 1324 | TTGAGGAAGTTGTAGC | NC_045512.2 | 3′→5′ | 28747 | 28762 |
| 1325 | CTTGAGGAAGTTGTAG | NC_045512.2 | 3′→5′ | 28748 | 28763 |
| 1326 | CCTTGAGGAAGTTGTA | NC_045512.2 | 3′→5′ | 28749 | 28764 |
| 1327 | TCCTTGAGGAAGTTGT | NC_045512.2 | 3′→5′ | 28750 | 28765 |
| 1328 | TTCCTTGAGGAAGTTG | NC_045512.2 | 3′→5′ | 28751 | 28766 |
| 1329 | GTTCCTTGAGGAAGTT | NC_045512.2 | 3′→5′ | 28752 | 28767 |
| 1330 | TGTTCCTTGAGGAAGT | NC_045512.2 | 3′→5′ | 28753 | 28768 |
| 1331 | TTGTTCCTTGAGGAAG | NC_045512.2 | 3′→5′ | 28754 | 28769 |
| 1332 | GTTGTTCCTTGAGGAA | NC_045512.2 | 3′→5′ | 28755 | 28770 |
| 1333 | TGTTGTTCCTTGAGGA | NC_045512.2 | 3′→5′ | 28756 | 28771 |
| 1334 | ATGTTGTTCCTTGAGG | NC_045512.2 | 3′→5′ | 28757 | 28772 |
| 1335 | AATGTTGTTCCTTGAG | NC_045512.2 | 3′→5′ | 28758 | 28773 |
| 1336 | CAATGTTGTTCCTTGA | NC_045512.2 | 3′→5′ | 28759 | 28774 |
| 1337 | GCAATGTTGTTCCTTG | NC_045512.2 | 3′→5′ | 28760 | 28775 |
| 1338 | GGCAATGTTGTTCCTT | NC_045512.2 | 3′→5′ | 28761 | 28776 |
| 1339 | TGGCAATGTTGTTCCT | NC_045512.2 | 3′→5′ | 28762 | 28777 |
| 1340 | TTGGCAATGTTGTTCC | NC_045512.2 | 3′→5′ | 28763 | 28778 |
| 1341 | TTTGGCAATGTTGTTC | NC_045512.2 | 3′→5′ | 28764 | 28779 |
| 1342 | TTTTGGCAATGTTGTT | NC_045512.2 | 3′→5′ | 28765 | 28780 |
| 1343 | CTTTTGGCAATGTTGT | NC_045512.2 | 3′→5′ | 28766 | 28781 |
| 1344 | CCTTTTGGCAATGTTG | NC_045512.2 | 3′→5′ | 28767 | 28782 |
| 1345 | GCCTTTTGGCAATGTT | NC_045512.2 | 3′→5′ | 28768 | 28783 |
| 1346 | AGCCTTTTGGCAATGT | NC_045512.2 | 3′→5′ | 28769 | 28784 |
| 1347 | AAGCCTTTTGGCAATG | NC_045512.2 | 3′→5′ | 28770 | 28785 |
| 1348 | GAAGCCTTTTGGCAAT | NC_045512.2 | 3′→5′ | 28771 | 28786 |
| 1349 | AGAAGCCTTTTGGCAA | NC_045512.2 | 3′→5′ | 28772 | 28787 |
| 1350 | TAGAAGCCTTTTGGCA | NC_045512.2 | 3′→5′ | 28773 | 28788 |
| 1351 | GTAGAAGCCTTTTGGC | NC_045512.2 | 3′→5′ | 28774 | 28789 |
| 1352 | CGTAGAAGCCTTTTGG | NC_045512.2 | 3′→5′ | 28775 | 28790 |
| 1353 | GCGTAGAAGCCTTTTG | NC_045512.2 | 3′→5′ | 28776 | 28791 |
| 1354 | TGCGTAGAAGCCTTTT | NC_045512.2 | 3′→5′ | 28777 | 28792 |
| 1355 | CTGCGTAGAAGCCTTT | NC_045512.2 | 3′→5′ | 28778 | 28793 |
| 1356 | TCTGCGTAGAAGCCTT | NC_045512.2 | 3′→5′ | 28779 | 28794 |
| 1357 | GACTGCCGCCTCTGCT | NC_045512.2 | 3′→5′ | 28799 | 28814 |
| 1358 | TGACTGCCGCCTCTGC | NC_045512.2 | 3′→5′ | 28800 | 28815 |
| 1359 | TTGACTGCCGCCTCTG | NC_045512.2 | 3′→5′ | 28801 | 28816 |
| 1360 | CTTGACTGCCGCCTCT | NC_045512.2 | 3′→5′ | 28802 | 28817 |
| 1361 | GCTTGACTGCCGCCTC | NC_045512.2 | 3′→5′ | 28803 | 28818 |
| 1362 | GGCTTGACTGCCGCCT | NC_045512.2 | 3′→5′ | 28804 | 28819 |
| 1363 | AGGCTTGACTGCCGCC | NC_045512.2 | 3′→5′ | 28805 | 28820 |
| 1364 | GAGGCTTGACTGCCGC | NC_045512.2 | 3′→5′ | 28806 | 28821 |
| 1365 | AGAGGCTTGACTGCCG | NC_045512.2 | 3′→5′ | 28807 | 28822 |
| 1366 | AAGAGGCTTGACTGCC | NC_045512.2 | 3′→5′ | 28808 | 28823 |
| 1367 | GAAGAGGCTTGACTGC | NC_045512.2 | 3′→5′ | 28809 | 28824 |
| 1368 | AGAAGAGGCTTGACTG | NC_045512.2 | 3′→5′ | 28810 | 28825 |
| 1369 | GAGAAGAGGCTTGACT | NC_045512.2 | 3′→5′ | 28811 | 28826 |
| 1370 | CGAGAAGAGGCTTGAC | NC_045512.2 | 3′→5′ | 28812 | 28827 |
| 1371 | AGTTGAATTTCTTGAA | NC_045512.2 | 3′→5′ | 28852 | 28867 |
| 1372 | GAGTTGAATTTCTTGA | NC_045512.2 | 3′→5′ | 28853 | 28868 |
| 1373 | GGAGTTGAATTTCTTG | NC_045512.2 | 3′→5′ | 28854 | 28869 |
| 1374 | TTCCCCTACTGCTGCC | NC_045512.2 | 3′→5′ | 28871 | 28886 |
| 1375 | GCTGGTTCAATCTGTC | NC_045512.2 | 3′→5′ | 28946 | 28961 |
| 1376 | AGCTGGTTCAATCTGT | NC_045512.2 | 3′→5′ | 28947 | 28962 |
| 1377 | AAGCTGGTTCAATCTG | NC_045512.2 | 3′→5′ | 28948 | 28963 |
| 1378 | CAAGCTGGTTCAATCT | NC_045512.2 | 3′→5′ | 28949 | 28964 |
| 1379 | TCAAGCTGGTTCAATC | NC_045512.2 | 3′→5′ | 28950 | 28965 |
| 1380 | CTCAAGCTGGTTCAAT | NC_045512.2 | 3′→5′ | 28951 | 28966 |
| 1381 | TCTCAAGCTGGTTCAA | NC_045512.2 | 3′→5′ | 28952 | 28967 |
| 1382 | CTCTCAAGCTGGTTCA | NC_045512.2 | 3′→5′ | 28953 | 28968 |
| 1383 | GCTCTCAAGCTGGTTC | NC_045512.2 | 3′→5′ | 28954 | 28969 |
| 1384 | TGCTCTCAAGCTGGTT | NC_045512.2 | 3′→5′ | 28955 | 28970 |
| 1385 | TTGCTCTCAAGCTGGT | NC_045512.2 | 3′→5′ | 28956 | 28971 |
| 1386 | TTTGCTCTCAAGCTGG | NC_045512.2 | 3′→5′ | 28957 | 28972 |
| 1387 | GTTGGCCTTTACCAGA | NC_045512.2 | 3′→5′ | 28976 | 28991 |
| 1388 | TGTTGGCCTTTACCAG | NC_045512.2 | 3′→5′ | 28977 | 28992 |
| 1389 | TTGTTGGCCTTTACCA | NC_045512.2 | 3′→5′ | 28978 | 28993 |
| 1390 | GTTGTTGGCCTTTACC | NC_045512.2 | 3′→5′ | 28979 | 28994 |
| 1391 | TGTTGTTGGCCTTTAC | NC_045512.2 | 3′→5′ | 28980 | 28995 |
| 1392 | TTGTTGTTGGCCTTTA | NC_045512.2 | 3′→5′ | 28981 | 28996 |
| 1393 | GTTGTTGTTGGCCTTT | NC_045512.2 | 3′→5′ | 28982 | 28997 |
| 1394 | TGTTGTTGTTGGCCTT | NC_045512.2 | 3′→5′ | 28983 | 28998 |
| 1395 | TTGTTGTTGTTGGCCT | NC_045512.2 | 3′→5′ | 28984 | 28999 |
| 1396 | CTTGTTGTTGTTGGCC | NC_045512.2 | 3′→5′ | 28985 | 29000 |
| 1397 | CCTTGTTGTTGTTGGC | NC_045512.2 | 3′→5′ | 28986 | 29001 |
| 1398 | GCCTTGTTGTTGTTGG | NC_045512.2 | 3′→5′ | 28987 | 29002 |
| 1399 | GGCCTTGTTGTTGTTG | NC_045512.2 | 3′→5′ | 28988 | 29003 |
| 1400 | TGGCCTTGTTGTTGTT | NC_045512.2 | 3′→5′ | 28989 | 29004 |
| 1401 | TTGGCCTTGTTGTTGT | NC_045512.2 | 3′→5′ | 28990 | 29005 |
| 1402 | TTTGGCCTTGTTGTTG | NC_045512.2 | 3′→5′ | 28991 | 29006 |
| 1403 | GTTTGGCCTTGTTGTT | NC_045512.2 | 3′→5′ | 28992 | 29007 |
| 1404 | AGTTTGGCCTTGTTGT | NC_045512.2 | 3′→5′ | 28993 | 29008 |
| 1405 | CAGTTTGGCCTTGTTG | NC_045512.2 | 3′→5′ | 28994 | 29009 |
| 1406 | ACAGTTTGGCCTTGTT | NC_045512.2 | 3′→5′ | 28995 | 29010 |
| 1407 | GACAGTTTGGCCTTGT | NC_045512.2 | 3′→5′ | 28996 | 29011 |
| 1408 | TGACAGTTTGGCCTTG | NC_045512.2 | 3′→5′ | 28997 | 29012 |
| 1409 | GTGACAGTTTGGCCTT | NC_045512.2 | 3′→5′ | 28998 | 29013 |
| 1410 | AGTGACAGTTTGGCCT | NC_045512.2 | 3′→5′ | 28999 | 29014 |
| 1411 | TAGTGACAGTTTGGCC | NC_045512.2 | 3′→5′ | 29000 | 29015 |
| 1412 | TTAGTGACAGTTTGGC | NC_045512.2 | 3′→5′ | 29001 | 29016 |
| 1413 | CTTAGTGACAGTTTGG | NC_045512.2 | 3′→5′ | 29002 | 29017 |
| 1414 | TCTTAGTGACAGTTTG | NC_045512.2 | 3′→5′ | 29003 | 29018 |
| 1415 | TTCTTAGTGACAGTTT | NC_045512.2 | 3′→5′ | 29004 | 29019 |
| 1416 | TTTCTTAGTGACAGTT | NC_045512.2 | 3′→5′ | 29005 | 29020 |
| 1417 | ATTTCTTAGTGACAGT | NC_045512.2 | 3′→5′ | 29006 | 29021 |
| 1418 | GATTTCTTAGTGACAG | NC_045512.2 | 3′→5′ | 29007 | 29022 |
| 1419 | AGATTTCTTAGTGACA | NC_045512.2 | 3′→5′ | 29008 | 29023 |
| 1420 | CAGATTTCTTAGTGAC | NC_045512.2 | 3′→5′ | 29009 | 29024 |
| 1421 | GCAGATTTCTTAGTGA | NC_045512.2 | 3′→5′ | 29010 | 29025 |
| 1422 | AGCAGATTTCTTAGTG | NC_045512.2 | 3′→5′ | 29011 | 29026 |
| 1423 | CAGCAGATTTCTTAGT | NC_045512.2 | 3′→5′ | 29012 | 29027 |
| 1424 | GCAGCAGATTTCTTAG | NC_045512.2 | 3′→5′ | 29013 | 29028 |
| 1425 | AGCAGCAGATTTCTTA | NC_045512.2 | 3′→5′ | 29014 | 29029 |
| 1426 | CAGCAGCAGATTTCTT | NC_045512.2 | 3′→5′ | 29015 | 29030 |
| 1427 | TCAGCAGCAGATTTCT | NC_045512.2 | 3′→5′ | 29016 | 29031 |
| 1428 | CTCAGCAGCAGATTTC | NC_045512.2 | 3′→5′ | 29017 | 29032 |
| 1429 | CCTCAGCAGCAGATTT | NC_045512.2 | 3′→5′ | 29018 | 29033 |
| 1430 | GCCTCAGCAGCAGATT | NC_045512.2 | 3′→5′ | 29019 | 29034 |
| 1431 | TTCCTTGTCTGATTAG | NC_045512.2 | 3′→5′ | 29144 | 29159 |
| 1432 | GTTCCTTGTCTGATTA | NC_045512.2 | 3′→5′ | 29145 | 29160 |
| 1433 | AGTTCCTTGTCTGATT | NC_045512.2 | 3′→5′ | 29146 | 29161 |
| 1434 | CAGTTCCTTGTCTGAT | NC_045512.2 | 3′→5′ | 29147 | 29162 |
| 1435 | TCAGTTCCTTGTCTGA | NC_045512.2 | 3′→5′ | 29148 | 29163 |
| 1436 | ATCAGTTCCTTGTCTG | NC_045512.2 | 3′→5′ | 29149 | 29164 |
| 1437 | AATCAGTTCCTTGTCT | NC_045512.2 | 3′→5′ | 29150 | 29165 |
| 1438 | TAATCAGTTCCTTGTC | NC_045512.2 | 3′→5′ | 29151 | 29166 |
| 1439 | GTAATCAGTTCCTTGT | NC_045512.2 | 3′→5′ | 29152 | 29167 |
| 1440 | TGTAATCAGTTCCTTG | NC_045512.2 | 3′→5′ | 29153 | 29168 |
| 1441 | TTGTAATCAGTTCCTT | NC_045512.2 | 3′→5′ | 29154 | 29169 |
| 1442 | TTTGTAATCAGTTCCT | NC_045512.2 | 3′→5′ | 29155 | 29170 |
| 1443 | GTTTGTAATCAGTTCC | NC_045512.2 | 3′→5′ | 29156 | 29171 |
| 1444 | TGTTTGTAATCAGTTC | NC_045512.2 | 3′→5′ | 29157 | 29172 |
| 1445 | GTGCAATTTGCGGCCA | NC_045512.2 | 3′→5′ | 29174 | 29189 |
| 1446 | TGTGCAATTTGCGGCC | NC_045512.2 | 3′→5′ | 29175 | 29190 |
| 1447 | TTGTGCAATTTGCGGC | NC_045512.2 | 3′→5′ | 29176 | 29191 |
| 1448 | ATTGTGCAATTTGCGG | NC_045512.2 | 3′→5′ | 29177 | 29192 |
| 1449 | AATTGTGCAATTTGCG | NC_045512.2 | 3′→5′ | 29178 | 29193 |
| 1450 | AAATTGTGCAATTTGC | NC_045512.2 | 3′→5′ | 29179 | 29194 |
| 1451 | CAAATTGTGCAATTTG | NC_045512.2 | 3′→5′ | 29180 | 29195 |
| 1452 | GCAAATTGTGCAATTT | NC_045512.2 | 3′→5′ | 29181 | 29196 |
| 1453 | CTTCCATGCCAATGCG | NC_045512.2 | 3′→5′ | 29228 | 29243 |
| 1454 | ACTTCCATGCCAATGC | NC_045512.2 | 3′→5′ | 29229 | 29244 |
| 1455 | GACTTCCATGCCAATG | NC_045512.2 | 3′→5′ | 29230 | 29245 |
| 1456 | TGACTTCCATGCCAAT | NC_045512.2 | 3′→5′ | 29231 | 29246 |
| 1457 | GTGACTTCCATGCCAA | NC_045512.2 | 3′→5′ | 29232 | 29247 |
| 1458 | TGTGACTTCCATGCCA | NC_045512.2 | 3′→5′ | 29233 | 29248 |
| 1459 | GTGTGACTTCCATGCC | NC_045512.2 | 3′→5′ | 29234 | 29249 |
| 1460 | GGTGTGACTTCCATGC | NC_045512.2 | 3′→5′ | 29235 | 29250 |
| 1461 | AGGTGTGACTTCCATG | NC_045512.2 | 3′→5′ | 29236 | 29251 |
| 1462 | AAGGTGTGACTTCCAT | NC_045512.2 | 3′→5′ | 29237 | 29252 |
| 1463 | GAAGGTGTGACTTCCA | NC_045512.2 | 3′→5′ | 29238 | 29253 |
| 1464 | CGAAGGTGTGACTTCC | NC_045512.2 | 3′→5′ | 29239 | 29254 |
| 1465 | CCGAAGGTGTGACTTC | NC_045512.2 | 3′→5′ | 29240 | 29255 |
| 1466 | CCCGAAGGTGTGACTT | NC_045512.2 | 3′→5′ | 29241 | 29256 |
| 1467 | TCCCGAAGGTGTGACT | NC_045512.2 | 3′→5′ | 29242 | 29257 |
| 1468 | TTCCCGAAGGTGTGAC | NC_045512.2 | 3′→5′ | 29243 | 29258 |
| 1469 | GTTCCCGAAGGTGTGA | NC_045512.2 | 3′→5′ | 29244 | 29259 |
| 1470 | CTTTGTCATCCAATTT | NC_045512.2 | 3′→5′ | 29285 | 29300 |
| 1471 | TCTTTGTCATCCAATT | NC_045512.2 | 3′→5′ | 29286 | 29301 |
| 1472 | ATCTTTGTCATCCAAT | NC_045512.2 | 3′→5′ | 29287 | 29302 |
| 1473 | GATCTTTGTCATCCAA | NC_045512.2 | 3′→5′ | 29288 | 29303 |
| 1474 | GGATCTTTGTCATCCA | NC_045512.2 | 3′→5′ | 29289 | 29304 |
| 1475 | TGGATCTTTGTCATCC | NC_045512.2 | 3′→5′ | 29290 | 29305 |
| 1476 | TTTTGTATGCGTCAAT | NC_045512.2 | 3′→5′ | 29342 | 29357 |
| 1477 | GTTTTGTATGCGTCAA | NC_045512.2 | 3′→5′ | 29343 | 29358 |
| 1478 | TGTTTTGTATGCGTCA | NC_045512.2 | 3′→5′ | 29344 | 29359 |
| 1479 | ATGTTTTGTATGCGTC | NC_045512.2 | 3′→5′ | 29345 | 29360 |
| 1480 | AATGTTTTGTATGCGT | NC_045512.2 | 3′→5′ | 29346 | 29361 |
| 1481 | GAATGTTTTGTATGCG | NC_045512.2 | 3′→5′ | 29347 | 29362 |
| 1482 | GGAATGTTTTGTATGC | NC_045512.2 | 3′→5′ | 29348 | 29363 |
| 1483 | GGGAATGTTTTGTATG | NC_045512.2 | 3′→5′ | 29349 | 29364 |
| 1484 | TGGGAATGTTTTGTAT | NC_045512.2 | 3′→5′ | 29350 | 29365 |
| 1485 | GTGGGAATGTTTTGTA | NC_045512.2 | 3′→5′ | 29351 | 29366 |
| 1486 | GGTGGGAATGTTTTGT | NC_045512.2 | 3′→5′ | 29352 | 29367 |
| 1487 | TGGTGGGAATGTTTTG | NC_045512.2 | 3′→5′ | 29353 | 29368 |
| 1488 | TTGGTGGGAATGTTTT | NC_045512.2 | 3′→5′ | 29354 | 29369 |
| 1489 | GTTGGTGGGAATGTTT | NC_045512.2 | 3′→5′ | 29355 | 29370 |
| 1490 | TGTTGGTGGGAATGTT | NC_045512.2 | 3′→5′ | 29356 | 29371 |
| 1491 | CTGTTGGTGGGAATGT | NC_045512.2 | 3′→5′ | 29357 | 29372 |
| 1492 | TCTGTTGGTGGGAATG | NC_045512.2 | 3′→5′ | 29358 | 29373 |
| 1493 | CTCTGTTGGTGGGAAT | NC_045512.2 | 3′→5′ | 29359 | 29374 |
| 1494 | GCTCTGTTGGTGGGAA | NC_045512.2 | 3′→5′ | 29360 | 29375 |
| 1495 | GGCTCTGTTGGTGGGA | NC_045512.2 | 3′→5′ | 29361 | 29376 |
| 1496 | AGGCTCTGTTGGTGGG | NC_045512.2 | 3′→5′ | 29362 | 29377 |
| 1497 | TAGGCTCTGTTGGTGG | NC_045512.2 | 3′→5′ | 29363 | 29378 |
| 1498 | TTAGGCTCTGTTGGTG | NC_045512.2 | 3′→5′ | 29364 | 29379 |
| 1499 | TTTAGGCTCTGTTGGT | NC_045512.2 | 3′→5′ | 29365 | 29380 |
| 1500 | TTTTAGGCTCTGTTGG | NC_045512.2 | 3′→5′ | 29366 | 29381 |
| 1501 | TTTTTAGGCTCTGTTG | NC_045512.2 | 3′→5′ | 29367 | 29382 |
| 1502 | CTTTTTAGGCTCTGTT | NC_045512.2 | 3′→5′ | 29368 | 29383 |
| 1503 | CCTTTTTAGGCTCTGT | NC_045512.2 | 3′→5′ | 29369 | 29384 |
| 1504 | TCCTTTTTAGGCTCTG | NC_045512.2 | 3′→5′ | 29370 | 29385 |
| 1505 | GTCCTTTTTAGGCTCT | NC_045512.2 | 3′→5′ | 29371 | 29386 |
| 1506 | TGTCCTTTTTAGGCTC | NC_045512.2 | 3′→5′ | 29372 | 29387 |
| 1507 | TTGTCCTTTTTAGGCT | NC_045512.2 | 3′→5′ | 29373 | 29388 |
| 1508 | TTTGTCCTTTTTAGGC | NC_045512.2 | 3′→5′ | 29374 | 29389 |
| 1509 | TTTTGTCCTTTTTAGG | NC_045512.2 | 3′→5′ | 29375 | 29390 |
| 1510 | TTTTTGTCCTTTTTAG | NC_045512.2 | 3′→5′ | 29376 | 29391 |
| 1511 | CTTTTTGTCCTTTTTA | NC_045512.2 | 3′→5′ | 29377 | 29392 |
| 1512 | TCTTTTTGTCCTTTTT | NC_045512.2 | 3′→5′ | 29378 | 29393 |
| 1513 | TTCTTTTTGTCCTTTT | NC_045512.2 | 3′→5′ | 29379 | 29394 |
| 1514 | GAAGAAGAGTCACAGT | NC_045512.2 | 3′→5′ | 29444 | 29459 |
| 1515 | GGAAGAAGAGTCACAG | NC_045512.2 | 3′→5′ | 29445 | 29460 |
| 1516 | AGGAAGAAGAGTCACA | NC_045512.2 | 3′→5′ | 29446 | 29461 |
| 1517 | CAGGAAGAAGAGTCAC | NC_045512.2 | 3′→5′ | 29447 | 29462 |
| 1518 | GCAGGAAGAAGAGTCA | NC_045512.2 | 3′→5′ | 29448 | 29463 |
| 1519 | TCTGCCTTGTGTGGTC | NC_045512.2 | 3′→5′ | 29543 | 29558 |
| 1520 | ATCTGCCTTGTGTGGT | NC_045512.2 | 3′→5′ | 29544 | 29559 |
| 1521 | CATCTGCCTTGTGTGG | NC_045512.2 | 3′→5′ | 29545 | 29560 |
| 1522 | CCATCTGCCTTGTGTG | NC_045512.2 | 3′→5′ | 29546 | 29561 |
| 1523 | CCCATCTGCCTTGTGT | NC_045512.2 | 3′→5′ | 29547 | 29562 |
| 1524 | GCCCATCTGCCTTGTG | NC_045512.2 | 3′→5′ | 29548 | 29563 |
| 1525 | AGCCCATCTGCCTTGT | NC_045512.2 | 3′→5′ | 29549 | 29564 |
| 1526 | TAGCCCATCTGCCTTG | NC_045512.2 | 3′→5′ | 29550 | 29565 |
| 1527 | ATAGCCCATCTGCCTT | NC_045512.2 | 3′→5′ | 29551 | 29566 |
| 1528 | CACAAGAGTAGACTAT | NC_045512.2 | 3′→5′ | 29598 | 29613 |
| 1529 | GCACAAGAGTAGACTA | NC_045512.2 | 3′→5′ | 29599 | 29614 |
| 1530 | TGCACAAGAGTAGACT | NC_045512.2 | 3′→5′ | 29600 | 29615 |
| 1531 | CTGCACAAGAGTAGAC | NC_045512.2 | 3′→5′ | 29601 | 29616 |
| 1532 | TCTGCACAAGAGTAGA | NC_045512.2 | 3′→5′ | 29602 | 29617 |
| 1533 | TTCTGCACAAGAGTAG | NC_045512.2 | 3′→5′ | 29603 | 29618 |
| 1534 | ATTCTGCACAAGAGTA | NC_045512.2 | 3′→5′ | 29604 | 29619 |
| 1535 | CATTCTGCACAAGAGT | NC_045512.2 | 3′→5′ | 29605 | 29620 |
| 1536 | TCATTCTGCACAAGAG | NC_045512.2 | 3′→5′ | 29606 | 29621 |
| 1537 | TTCATTCTGCACAAGA | NC_045512.2 | 3′→5′ | 29607 | 29622 |
| 1538 | ATTCATTCTGCACAAG | NC_045512.2 | 3′→5′ | 29608 | 29623 |
| 1539 | AATTCATTCTGCACAA | NC_045512.2 | 3′→5′ | 29609 | 29624 |
| 1540 | GAATTCATTCTGCACA | NC_045512.2 | 3′→5′ | 29610 | 29625 |
| 1541 | AGAATTCATTCTGCAC | NC_045512.2 | 3′→5′ | 29611 | 29626 |
| 1542 | GAGAATTCATTCTGCA | NC_045512.2 | 3′→5′ | 29612 | 29627 |
| 1543 | CGAGAATTCATTCTGC | NC_045512.2 | 3′→5′ | 29613 | 29628 |
| 1544 | ACGAGAATTCATTCTG | NC_045512.2 | 3′→5′ | 29614 | 29629 |
| 1545 | TACGAGAATTCATTCT | NC_045512.2 | 3′→5′ | 29615 | 29630 |
| 1546 | AGATTAAAGTTAACTA | NC_045512.2 | 3′→5′ | 29652 | 29667 |
| 1547 | GAGATTAAAGTTAACT | NC_045512.2 | 3′→5′ | 29653 | 29668 |
| 1548 | TGAGATTAAAGTTAAC | NC_045512.2 | 3′→5′ | 29654 | 29669 |
| 1549 | GTGAGATTAAAGTTAA | NC_045512.2 | 3′→5′ | 29655 | 29670 |
| 1550 | TGTGAGATTAAAGTTA | NC_045512.2 | 3′→5′ | 29656 | 29671 |
| 1551 | ATGTGAGATTAAAGTT | NC_045512.2 | 3′→5′ | 29657 | 29672 |
| 1552 | TATGTGAGATTAAAGT | NC_045512.2 | 3′→5′ | 29658 | 29673 |
| 1553 | CTATGTGAGATTAAAG | NC_045512.2 | 3′→5′ | 29659 | 29674 |
| 1554 | GCTATGTGAGATTAAA | NC_045512.2 | 3′→5′ | 29660 | 29675 |
| 1555 | TGCTATGTGAGATTAA | NC_045512.2 | 3′→5′ | 29661 | 29676 |
| 1556 | TTGCTATGTGAGATTA | NC_045512.2 | 3′→5′ | 29662 | 29677 |
| 1557 | ATTGCTATGTGAGATT | NC_045512.2 | 3′→5′ | 29663 | 29678 |
| 1558 | GATTGCTATGTGAGAT | NC_045512.2 | 3′→5′ | 29664 | 29679 |
| 1559 | AGATTGCTATGTGAGA | NC_045512.2 | 3′→5′ | 29665 | 29680 |
| 1560 | AAGATTGCTATGTGAG | NC_045512.2 | 3′→5′ | 29666 | 29681 |
| 1561 | AAAGATTGCTATGTGA | NC_045512.2 | 3′→5′ | 29667 | 29682 |
| 1562 | TAAAGATTGCTATGTG | NC_045512.2 | 3′→5′ | 29668 | 29683 |
| 1563 | TTAAAGATTGCTATGT | NC_045512.2 | 3′→5′ | 29669 | 29684 |
| 1564 | ATTAAAGATTGCTATG | NC_045512.2 | 3′→5′ | 29670 | 29685 |
| 1565 | GATTAAAGATTGCTAT | NC_045512.2 | 3′→5′ | 29671 | 29686 |
| 1566 | TGATTAAAGATTGCTA | NC_045512.2 | 3′→5′ | 29672 | 29687 |
| 1567 | TCCCTAATGTTACACA | NC_045512.2 | 3′→5′ | 29689 | 29704 |
| 1568 | CTCCCTAATGTTACAC | NC_045512.2 | 3′→5′ | 29690 | 29705 |
| 1569 | CCTCCCTAATGTTACA | NC_045512.2 | 3′→5′ | 29691 | 29706 |
| 1570 | TCCTCCCTAATGTTAC | NC_045512.2 | 3′→5′ | 29692 | 29707 |
| 1571 | GTCCTCCCTAATGTTA | NC_045512.2 | 3′→5′ | 29693 | 29708 |
| 1572 | AGTCCTCCCTAATGTT | NC_045512.2 | 3′→5′ | 29694 | 29709 |
| 1573 | AAGTCCTCCCTAATGT | NC_045512.2 | 3′→5′ | 29695 | 29710 |
| 1574 | CAAGTCCTCCCTAATG | NC_045512.2 | 3′→5′ | 29696 | 29711 |
| 1575 | TCAAGTCCTCCCTAAT | NC_045512.2 | 3′→5′ | 29697 | 29712 |
| 1576 | TTCAAGTCCTCCCTAA | NC_045512.2 | 3′→5′ | 29698 | 29713 |
| 1577 | TTTCAAGTCCTCCCTA | NC_045512.2 | 3′→5′ | 29699 | 29714 |
| 1578 | CTTTCAAGTCCTCCCT | NC_045512.2 | 3′→5′ | 29700 | 29715 |
| 1579 | TCTTTCAAGTCCTCCC | NC_045512.2 | 3′→5′ | 29701 | 29716 |
| 1580 | CTCTTTCAAGTCCTCC | NC_045512.2 | 3′→5′ | 29702 | 29717 |
| 1581 | GCTCTTTCAAGTCCTC | NC_045512.2 | 3′→5′ | 29703 | 29718 |
| 1582 | GGCTCTTTCAAGTCCT | NC_045512.2 | 3′→5′ | 29704 | 29719 |
| 1583 | TGGCTCTTTCAAGTCC | NC_045512.2 | 3′→5′ | 29705 | 29720 |
| 1584 | GTGGCTCTTTCAAGTC | NC_045512.2 | 3′→5′ | 29706 | 29721 |
| 1585 | GGTGGCTCTTTCAAGT | NC_045512.2 | 3′→5′ | 29707 | 29722 |
| 1586 | TGGTGGCTCTTTCAAG | NC_045512.2 | 3′→5′ | 29708 | 29723 |
| 1587 | GTGGTGGCTCTTTCAA | NC_045512.2 | 3′→5′ | 29709 | 29724 |
| 1588 | TGTGGTGGCTCTTTCA | NC_045512.2 | 3′→5′ | 29710 | 29725 |
| 1589 | ATGTGGTGGCTCTTTC | NC_045512.2 | 3′→5′ | 29711 | 29726 |
| 1590 | AATGTGGTGGCTCTTT | NC_045512.2 | 3′→5′ | 29712 | 29727 |
| 1591 | AAATGTGGTGGCTCTT | NC_045512.2 | 3′→5′ | 29713 | 29728 |
| 1592 | AAAATGTGGTGGCTCT | NC_045512.2 | 3′→5′ | 29714 | 29729 |
| 1593 | GAAAATGTGGTGGCTC | NC_045512.2 | 3′→5′ | 29715 | 29730 |
| 1594 | TGAAAATGTGGTGGCT | NC_045512.2 | 3′→5′ | 29716 | 29731 |
| 1595 | ACTCCGCGTGGCCTCG | NC_045512.2 | 3′→5′ | 29733 | 29748 |
| 1596 | TACTCCGCGTGGCCTC | NC_045512.2 | 3′→5′ | 29734 | 29749 |
| 1597 | GTACTCCGCGTGGCCT | NC_045512.2 | 3′→5′ | 29735 | 29750 |
| 1598 | CGTACTCCGCGTGGCC | NC_045512.2 | 3′→5′ | 29736 | 29751 |
| 1599 | TCGTACTCCGCGTGGC | NC_045512.2 | 3′→5′ | 29737 | 29752 |
| 1600 | ATCGTACTCCGCGTGG | NC_045512.2 | 3′→5′ | 29738 | 29753 |
| 1601 | GATCGTACTCCGCGTG | NC_045512.2 | 3′→5′ | 29739 | 29754 |
| 1602 | CGATCGTACTCCGCGT | NC_045512.2 | 3′→5′ | 29740 | 29755 |
| 1603 | TCGATCGTACTCCGCG | NC_045512.2 | 3′→5′ | 29741 | 29756 |
| 1604 | CTCGATCGTACTCCGC | NC_045512.2 | 3′→5′ | 29742 | 29757 |
| 1605 | AGCTCTCCCTAGCATT | NC_045512.2 | 3′→5′ | 29770 | 29785 |
| 1606 | CAGCTCTCCCTAGCAT | NC_045512.2 | 3′→5′ | 29771 | 29786 |
| 1607 | GCAGCTCTCCCTAGCA | NC_045512.2 | 3′→5′ | 29772 | 29787 |
| 1608 | GGCAGCTCTCCCTAGC | NC_045512.2 | 3′→5′ | 29773 | 29788 |
| 1609 | AGGCAGCTCTCCCTAG | NC_045512.2 | 3′→5′ | 29774 | 29789 |
| 1610 | TAGGCAGCTCTCCCTA | NC_045512.2 | 3′→5′ | 29775 | 29790 |
| 1611 | ATAGGCAGCTCTCCCT | NC_045512.2 | 3′→5′ | 29776 | 29791 |
| 1612 | TATAGGCAGCTCTCCC | NC_045512.2 | 3′→5′ | 29777 | 29792 |
| 1613 | ATATAGGCAGCTCTCC | NC_045512.2 | 3′→5′ | 29778 | 29793 |
| 1614 | CATATAGGCAGCTCTC | NC_045512.2 | 3′→5′ | 29779 | 29794 |
| 1615 | CCATATAGGCAGCTCT | NC_045512.2 | 3′→5′ | 29780 | 29795 |
| 1616 | TCCATATAGGCAGCTC | NC_045512.2 | 3′→5′ | 29781 | 29796 |
| 1617 | TTCCATATAGGCAGCT | NC_045512.2 | 3′→5′ | 29782 | 29797 |
| 1618 | CTTCCATATAGGCAGC | NC_045512.2 | 3′→5′ | 29783 | 29798 |
| 1619 | TCTTCCATATAGGCAG | NC_045512.2 | 3′→5′ | 29784 | 29799 |
| 1620 | CTCTTCCATATAGGCA | NC_045512.2 | 3′→5′ | 29785 | 29800 |
| 1621 | GCTCTTCCATATAGGC | NC_045512.2 | 3′→5′ | 29786 | 29801 |
| 1622 | GGCTCTTCCATATAGG | NC_045512.2 | 3′→5′ | 29787 | 29802 |
| 1623 | GGGCTCTTCCATATAG | NC_045512.2 | 3′→5′ | 29788 | 29803 |
| 1624 | AGGGCTCTTCCATATA | NC_045512.2 | 3′→5′ | 29789 | 29804 |
| 1625 | TAGGGCTCTTCCATAT | NC_045512.2 | 3′→5′ | 29790 | 29805 |
| 1626 | TTAGGGCTCTTCCATA | NC_045512.2 | 3′→5′ | 29791 | 29806 |
| 1627 | ATTAGGGCTCTTCCAT | NC_045512.2 | 3′→5′ | 29792 | 29807 |
| 1628 | CATTAGGGCTCTTCCA | NC_045512.2 | 3′→5′ | 29793 | 29808 |
| 1629 | ACATTAGGGCTCTTCC | NC_045512.2 | 3′→5′ | 29794 | 29809 |
| 1630 | CACATTAGGGCTCTTC | NC_045512.2 | 3′→5′ | 29795 | 29810 |
| 1631 | ACACATTAGGGCTCTT | NC_045512.2 | 3′→5′ | 29796 | 29811 |
| 1632 | TACACATTAGGGCTCT | NC_045512.2 | 3′→5′ | 29797 | 29812 |
| 1633 | TTACACATTAGGGCTC | NC_045512.2 | 3′→5′ | 29798 | 29813 |
| 1634 | TTTACACATTAGGGCT | NC_045512.2 | 3′→5′ | 29799 | 29814 |
| 1635 | TTTTACACATTAGGGC | NC_045512.2 | 3′→5′ | 29800 | 29815 |
| 1636 | ATTTTACACATTAGGG | NC_045512.2 | 3′→5′ | 29801 | 29816 |
| 1637 | AATTTTACACATTAGG | NC_045512.2 | 3′→5′ | 29802 | 29817 |
| 1638 | TAATTTTACACATTAG | NC_045512.2 | 3′→5′ | 29803 | 29818 |
| 1639 | TTAATTTTACACATTA | NC_045512.2 | 3′→5′ | 29804 | 29819 |
| 1640 | ATTAATTTTACACATT | NC_045512.2 | 3′→5′ | 29805 | 29820 |
| 1641 | AATTAATTTTACACAT | NC_045512.2 | 3′→5′ | 29806 | 29821 |
| 1642 | AAATTAATTTTACACA | NC_045512.2 | 3′→5′ | 29807 | 29822 |
| 1643 | AAAATTAATTTTACAC | NC_045512.2 | 3′→5′ | 29808 | 29823 |
| 1644 | TAAAATTAATTTTACA | NC_045512.2 | 3′→5′ | 29809 | 29824 |
| 1645 | CTAAAATTAATTTTAC | NC_045512.2 | 3′→5′ | 29810 | 29825 |
| 1646 | ACTAAAATTAATTTTA | NC_045512.2 | 3′→5′ | 29811 | 29826 |
| 1647 | TACTAAAATTAATTTT | NC_045512.2 | 3′→5′ | 29812 | 29827 |
| 1648 | CTACTAAAATTAATTT | NC_045512.2 | 3′→5′ | 29813 | 29828 |
| 1649 | TGTCAACGACAGTAATT | NC_045512.2 | 3′→5′ | 142 | 158 |
| 1650 | GACGAAACCGTAAGCAG | NC_045512.2 | 3′→5′ | 190 | 206 |
| 1651 | GGACGAAACCGTAAGCA | NC_045512.2 | 3′→5′ | 191 | 207 |
| 1652 | CGGACGAAACCGTAAGC | NC_045512.2 | 3′→5′ | 192 | 208 |
| 1653 | ACGGACGAAACCGTAAG | NC_045512.2 | 3′→5′ | 193 | 209 |
| 1654 | CACGGACGAAACCGTAA | NC_045512.2 | 3′→5′ | 194 | 210 |
| 1655 | ACACGGACGAAACCGTA | NC_045512.2 | 3′→5′ | 195 | 211 |
| 1656 | AACACGGACGAAACCGT | NC_045512.2 | 3′→5′ | 196 | 212 |
| 1657 | CAACACGGACGAAACCG | NC_045512.2 | 3′→5′ | 197 | 213 |
| 1658 | GCAACACGGACGAAACC | NC_045512.2 | 3′→5′ | 198 | 214 |
| 1659 | TGCAACACGGACGAAAC | NC_045512.2 | 3′→5′ | 199 | 215 |
| 1660 | CTGCAACACGGACGAAA | NC_045512.2 | 3′→5′ | 200 | 216 |
| 1661 | ACCCGGACGAAACCTAG | NC_045512.2 | 3′→5′ | 233 | 249 |
| 1662 | CACCCGGACGAAACCTA | NC_045512.2 | 3′→5′ | 234 | 250 |
| 1663 | ACACCCGGACGAAACCT | NC_045512.2 | 3′→5′ | 235 | 251 |
| 1664 | CACACCCGGACGAAACC | NC_045512.2 | 3′→5′ | 236 | 252 |
| 1665 | TCACACCCGGACGAAAC | NC_045512.2 | 3′→5′ | 237 | 253 |
| 1666 | GTCACACCCGGACGAAA | NC_045512.2 | 3′→5′ | 238 | 254 |
| 1667 | GGTCACACCCGGACGAA | NC_045512.2 | 3′→5′ | 239 | 255 |
| 1668 | CGGTCACACCCGGACGA | NC_045512.2 | 3′→5′ | 240 | 256 |
| 1669 | TCGGTCACACCCGGACG | NC_045512.2 | 3′→5′ | 241 | 257 |
| 1670 | TTCGGTCACACCCGGAC | NC_045512.2 | 3′→5′ | 242 | 258 |
| 1671 | TTTCGGTCACACCCGGA | NC_045512.2 | 3′→5′ | 243 | 259 |
| 1672 | CTTTCGGTCACACCCGG | NC_045512.2 | 3′→5′ | 244 | 260 |
| 1673 | CCTTTCGGTCACACCCG | NC_045512.2 | 3′→5′ | 245 | 261 |
| 1674 | ACCTTTCGGTCACACCC | NC_045512.2 | 3′→5′ | 246 | 262 |
| 1675 | TACCTTTCGGTCACACC | NC_045512.2 | 3′→5′ | 247 | 263 |
| 1676 | TTACCTTTCGGTCACAC | NC_045512.2 | 3′→5′ | 248 | 264 |
| 1677 | CTTACCTTTCGGTCACA | NC_045512.2 | 3′→5′ | 249 | 265 |
| 1678 | TCTTACCTTTCGGTCAC | NC_045512.2 | 3′→5′ | 250 | 266 |
| 1679 | ATCTTACCTTTCGGTCA | NC_045512.2 | 3′→5′ | 251 | 267 |
| 1680 | CATCTTACCTTTCGGTC | NC_045512.2 | 3′→5′ | 252 | 268 |
| 1681 | CCATCTTACCTTTCGGT | NC_045512.2 | 3′→5′ | 253 | 269 |
| 1682 | TCCATCTTACCTTTCGG | NC_045512.2 | 3′→5′ | 254 | 270 |
| 1683 | CTCCATCTTACCTTTCG | NC_045512.2 | 3′→5′ | 255 | 271 |
| 1684 | TCTCCATCTTACCTTTC | NC_045512.2 | 3′→5′ | 256 | 272 |
| 1685 | CTCTCCATCTTACCTTT | NC_045512.2 | 3′→5′ | 257 | 273 |
| 1686 | GCTCTCCATCTTACCTT | NC_045512.2 | 3′→5′ | 258 | 274 |
| 1687 | GGCTCTCCATCTTACCT | NC_045512.2 | 3′→5′ | 259 | 275 |
| 1688 | AGGCTCTCCATCTTACC | NC_045512.2 | 3′→5′ | 260 | 276 |
| 1689 | AAGGCTCTCCATCTTAC | NC_045512.2 | 3′→5′ | 261 | 277 |
| 1690 | CAAGGCTCTCCATCTTA | NC_045512.2 | 3′→5′ | 262 | 278 |
| 1691 | ACAAGGCTCTCCATCTT | NC_045512.2 | 3′→5′ | 263 | 279 |
| 1692 | GTGTGTTTTCTCGTTGA | NC_045512.2 | 3′→5′ | 288 | 304 |
| 1693 | CGTGTGTTTTCTCGTTG | NC_045512.2 | 3′→5′ | 289 | 305 |
| 1694 | ACGTGTGTTTTCTCGTT | NC_045512.2 | 3′→5′ | 290 | 306 |
| 1695 | GACGTGTGTTTTCTCGT | NC_045512.2 | 3′→5′ | 291 | 307 |
| 1696 | GGACGTGTGTTTTCTCG | NC_045512.2 | 3′→5′ | 292 | 308 |
| 1697 | TGGACGTGTGTTTTCTC | NC_045512.2 | 3′→5′ | 293 | 309 |
| 1698 | TTGGACGTGTGTTTTCT | NC_045512.2 | 3′→5′ | 294 | 310 |
| 1699 | GTTGGACGTGTGTTTTC | NC_045512.2 | 3′→5′ | 295 | 311 |
| 1700 | AGTTGGACGTGTGTTTT | NC_045512.2 | 3′→5′ | 296 | 312 |
| 1701 | GAGTTGGACGTGTGTTT | NC_045512.2 | 3′→5′ | 297 | 313 |
| 1702 | TGAGTTGGACGTGTGTT | NC_045512.2 | 3′→5′ | 298 | 314 |
| 1703 | CTGAGTTGGACGTGTGT | NC_045512.2 | 3′→5′ | 299 | 315 |
| 1704 | ACTGAGTTGGACGTGTG | NC_045512.2 | 3′→5′ | 300 | 316 |
| 1705 | AACTGAGTTGGACGTGT | NC_045512.2 | 3′→5′ | 301 | 317 |
| 1706 | AAACTGAGTTGGACGTG | NC_045512.2 | 3′→5′ | 302 | 318 |
| 1707 | CAAACTGAGTTGGACGT | NC_045512.2 | 3′→5′ | 303 | 319 |
| 1708 | GCAAACTGAGTTGGACG | NC_045512.2 | 3′→5′ | 304 | 320 |
| 1709 | GGCAAACTGAGTTGGAC | NC_045512.2 | 3′→5′ | 305 | 321 |
| 1710 | AGGCAAACTGAGTTGGA | NC_045512.2 | 3′→5′ | 306 | 322 |
| 1711 | CAGGCAAACTGAGTTGG | NC_045512.2 | 3′→5′ | 307 | 323 |
| 1712 | ACAGGCAAACTGAGTTG | NC_045512.2 | 3′→5′ | 308 | 324 |
| 1713 | ACATAGGGCTGTTCAAG | NC_045512.2 | 3′→5′ | 455 | 471 |
| 1714 | CACATAGGGCTGTTCAA | NC_045512.2 | 3′→5′ | 456 | 472 |
| 1715 | ACACATAGGGCTGTTCA | NC_045512.2 | 3′→5′ | 457 | 473 |
| 1716 | AACACATAGGGCTGTTC | NC_045512.2 | 3′→5′ | 458 | 474 |
| 1717 | GAACACATAGGGCTGTT | NC_045512.2 | 3′→5′ | 459 | 475 |
| 1718 | TGAACACATAGGGCTGT | NC_045512.2 | 3′→5′ | 460 | 476 |
| 1719 | ATGAACACATAGGGCTG | NC_045512.2 | 3′→5′ | 461 | 477 |
| 1720 | TTCTTACGAAGAAGAAC | NC_045512.2 | 3′→5′ | 626 | 642 |
| 1721 | GTTCTTACGAAGAAGAA | NC_045512.2 | 3′→5′ | 627 | 643 |
| 1722 | CGTTCTTACGAAGAAGA | NC_045512.2 | 3′→5′ | 628 | 644 |
| 1723 | CCGTTCTTACGAAGAAG | NC_045512.2 | 3′→5′ | 629 | 645 |
| 1724 | ACCGTTCTTACGAAGAA | NC_045512.2 | 3′→5′ | 630 | 646 |
| 1725 | TACCGTTCTTACGAAGA | NC_045512.2 | 3′→5′ | 631 | 647 |
| 1726 | TTACCGTTCTTACGAAG | NC_045512.2 | 3′→5′ | 632 | 648 |
| 1727 | ATTACCGTTCTTACGAA | NC_045512.2 | 3′→5′ | 633 | 649 |
| 1728 | TATTACCGTTCTTACGA | NC_045512.2 | 3′→5′ | 634 | 650 |
| 1729 | TTATTACCGTTCTTACG | NC_045512.2 | 3′→5′ | 635 | 651 |
| 1730 | TCAGTGCCAAGCTCGTC | NC_045512.2 | 3′→5′ | 704 | 720 |
| 1731 | ATCAGTGCCAAGCTCGT | NC_045512.2 | 3′→5′ | 705 | 721 |
| 1732 | GATCAGTGCCAAGCTCG | NC_045512.2 | 3′→5′ | 706 | 722 |
| 1733 | GGATCAGTGCCAAGCTC | NC_045512.2 | 3′→5′ | 707 | 723 |
| 1734 | AACACACAGCCTCCAAA | NC_045512.2 | 3′→5′ | 1481 | 1497 |
| 1735 | TAAGTTTTAAATAACCA | NC_045512.2 | 3′→5′ | 3352 | 3368 |
| 1736 | GTAAGTTTTAAATAACC | NC_045512.2 | 3′→5′ | 3353 | 3369 |
| 1737 | AGTAAGTTTTAAATAAC | NC_045512.2 | 3′→5′ | 3354 | 3370 |
| 1738 | CAGTAAGTTTTAAATAA | NC_045512.2 | 3′→5′ | 3355 | 3371 |
| 1739 | TCAGTAAGTTTTAAATA | NC_045512.2 | 3′→5′ | 3356 | 3372 |
| 1740 | GTCAGTAAGTTTTAAAT | NC_045512.2 | 3′→5′ | 3357 | 3373 |
| 1741 | TGTCAGTAAGTTTTAAA | NC_045512.2 | 3′→5′ | 3358 | 3374 |
| 1742 | TTGTCAGTAAGTTTTAA | NC_045512.2 | 3′→5′ | 3359 | 3375 |
| 1743 | ATTGTCAGTAAGTTTTA | NC_045512.2 | 3′→5′ | 3360 | 3376 |
| 1744 | CATTGTCAGTAAGTTTT | NC_045512.2 | 3′→5′ | 3361 | 3377 |
| 1745 | ACATTGTCAGTAAGTTT | NC_045512.2 | 3′→5′ | 3362 | 3378 |
| 1746 | AACTTAGTTTCTTCCAG | NC_045512.2 | 3′→5′ | 3992 | 4008 |
| 1747 | GCACAAAAGTTAGCAGC | NC_045512.2 | 3′→5′ | 5384 | 5400 |
| 1748 | TGCACAAAAGTTAGCAG | NC_045512.2 | 3′→5′ | 5385 | 5401 |
| 1749 | GTGCACAAAAGTTAGCA | NC_045512.2 | 3′→5′ | 5386 | 5402 |
| 1750 | AGTGCACAAAAGTTAGC | NC_045512.2 | 3′→5′ | 5387 | 5403 |
| 1751 | CCACTACTTCTTCAGAG | NC_045512.2 | 3′→5′ | 6406 | 6422 |
| 1752 | TCCACTACTTCTTCAGA | NC_045512.2 | 3′→5′ | 6407 | 6423 |
| 1753 | TTCCACTACTTCTTCAG | NC_045512.2 | 3′→5′ | 6408 | 6424 |
| 1754 | TTTCCACTACTTCTTCA | NC_045512.2 | 3′→5′ | 6409 | 6425 |
| 1755 | TTTTCCACTACTTCTTC | NC_045512.2 | 3′→5′ | 6410 | 6426 |
| 1756 | ATTTTCCACTACTTCTT | NC_045512.2 | 3′→5′ | 6411 | 6427 |
| 1757 | GATTTTCCACTACTTCT | NC_045512.2 | 3′→5′ | 6412 | 6428 |
| 1758 | GGATTTTCCACTACTTC | NC_045512.2 | 3′→5′ | 6413 | 6429 |
| 1759 | AGGATTTTCCACTACTT | NC_045512.2 | 3′→5′ | 6414 | 6430 |
| 1760 | TAGGATTTTCCACTACT | NC_045512.2 | 3′→5′ | 6415 | 6431 |
| 1761 | GTAGGATTTTCCACTAC | NC_045512.2 | 3′→5′ | 6416 | 6432 |
| 1762 | GGTAGGATTTTCCACTA | NC_045512.2 | 3′→5′ | 6417 | 6433 |
| 1763 | ACTTCGGTAGTTTTCAC | NC_045512.2 | 3′→5′ | 6461 | 6477 |
| 1764 | AACTTCGGTAGTTTTCA | NC_045512.2 | 3′→5′ | 6462 | 6478 |
| 1765 | CAACTTCGGTAGTTTTC | NC_045512.2 | 3′→5′ | 6463 | 6479 |
| 1766 | ACAACTTCGGTAGTTTT | NC_045512.2 | 3′→5′ | 6464 | 6480 |
| 1767 | TACAACTTCGGTAGTTT | NC_045512.2 | 3′→5′ | 6465 | 6481 |
| 1768 | CTACAACTTCGGTAGTT | NC_045512.2 | 3′→5′ | 6466 | 6482 |
| 1769 | CCTACAACTTCGGTAGT | NC_045512.2 | 3′→5′ | 6467 | 6483 |
| 1770 | TCATTAGGTTTCTTAAT | NC_045512.2 | 3′→5′ | 6584 | 6600 |
| 1771 | TTAACAATAGTTGTACA | NC_045512.2 | 3′→5′ | 7532 | 7548 |
| 1772 | ATTAACAATAGTTGTAC | NC_045512.2 | 3′→5′ | 7533 | 7549 |
| 1773 | CATTAACAATAGTTGTA | NC_045512.2 | 3′→5′ | 7534 | 7550 |
| 1774 | CCATTAACAATAGTTGT | NC_045512.2 | 3′→5′ | 7535 | 7551 |
| 1775 | ATTGGTCTTTTAAACTG | NC_045512.2 | 3′→5′ | 7691 | 7707 |
| 1776 | TTGTGACTTTTTGCTAC | NC_045512.2 | 3′→5′ | 8372 | 8388 |
| 1777 | TGTCACGAGTGACACCA | NC_045512.2 | 3′→5′ | 8707 | 8723 |
| 1778 | ATGTCACGAGTGACACC | NC_045512.2 | 3′→5′ | 8708 | 8724 |
| 1779 | TGTCAAGTACAAGTAAA | NC_045512.2 | 3′→5′ | 9588 | 9604 |
| 1780 | ATGTCAAGTACAAGTAA | NC_045512.2 | 3′→5′ | 9589 | 9605 |
| 1781 | AATGTCAAGTACAAGTA | NC_045512.2 | 3′→5′ | 9590 | 9606 |
| 1782 | ACATGTCTTGGACAGTA | NC_045512.2 | 3′→5′ | 10163 | 10179 |
| 1783 | GATGGTGAACCATTGTA | NC_045512.2 | 3′→5′ | 10406 | 10422 |
| 1784 | CCAACACTACCACATGA | NC_045512.2 | 3′→5′ | 10484 | 10500 |
| 1785 | ACCAACACTACCACATG | NC_045512.2 | 3′→5′ | 10485 | 10501 |
| 1786 | AACCAACACTACCACAT | NC_045512.2 | 3′→5′ | 10486 | 10502 |
| 1787 | AAACCAACACTACCACA | NC_045512.2 | 3′→5′ | 10487 | 10503 |
| 1788 | AAAACCAACACTACCAC | NC_045512.2 | 3′→5′ | 10488 | 10504 |
| 1789 | TAAAACCAACACTACCA | NC_045512.2 | 3′→5′ | 10489 | 10505 |
| 1790 | TTAAAACCAACACTACC | NC_045512.2 | 3′→5′ | 10490 | 10506 |
| 1791 | GTTAAAACCAACACTAC | NC_045512.2 | 3′→5′ | 10491 | 10507 |
| 1792 | TGTTAAAACCAACACTA | NC_045512.2 | 3′→5′ | 10492 | 10508 |
| 1793 | ATGTTAAAACCAACACT | NC_045512.2 | 3′→5′ | 10493 | 10509 |
| 1794 | CCTAAGAAACAATAAAC | NC_045512.2 | 3′→5′ | 11609 | 11625 |
| 1795 | GCCTAAGAAACAATAAA | NC_045512.2 | 3′→5′ | 11610 | 11626 |
| 1796 | AGCCTAAGAAACAATAA | NC_045512.2 | 3′→5′ | 11611 | 11627 |
| 1797 | TAGCCTAAGAAACAATA | NC_045512.2 | 3′→5′ | 11612 | 11628 |
| 1798 | ATAGCCTAAGAAACAAT | NC_045512.2 | 3′→5′ | 11613 | 11629 |
| 1799 | AATAGCCTAAGAAACAA | NC_045512.2 | 3′→5′ | 11614 | 11630 |
| 1800 | TCATAAACACCAAGAGT | NC_045512.2 | 3′→5′ | 11684 | 11700 |
| 1801 | ATTTTAGACTGTACAGT | NC_045512.2 | 3′→5′ | 11834 | 11850 |
| 1802 | CATTTTAGACTGTACAG | NC_045512.2 | 3′→5′ | 11835 | 11851 |
| 1803 | ACATTTTAGACTGTACA | NC_045512.2 | 3′→5′ | 11836 | 11852 |
| 1804 | GACATTTTAGACTGTAC | NC_045512.2 | 3′→5′ | 11837 | 11853 |
| 1805 | ACAGCACCCTGCATGGA | NC_045512.2 | 3′→5′ | 12023 | 12039 |
| 1806 | TACAGCACCCTGCATGG | NC_045512.2 | 3′→5′ | 12024 | 12040 |
| 1807 | CTACAGCACCCTGCATG | NC_045512.2 | 3′→5′ | 12025 | 12041 |
| 1808 | TCTACAGCACCCTGCAT | NC_045512.2 | 3′→5′ | 12026 | 12042 |
| 1809 | GTCTACAGCACCCTGCA | NC_045512.2 | 3′→5′ | 12027 | 12043 |
| 1810 | TGTCTACAGCACCCTGC | NC_045512.2 | 3′→5′ | 12028 | 12044 |
| 1811 | ATGTCTACAGCACCCTG | NC_045512.2 | 3′→5′ | 12029 | 12045 |
| 1812 | ACTTCAGAATCACCATT | NC_045512.2 | 3′→5′ | 12173 | 12189 |
| 1813 | TTAGCCACATTCAAAGA | NC_045512.2 | 3′→5′ | 12212 | 12228 |
| 1814 | TTTAGCCACATTCAAAG | NC_045512.2 | 3′→5′ | 12213 | 12229 |
| 1815 | ATTTAGCCACATTCAAA | NC_045512.2 | 3′→5′ | 12214 | 12230 |
| 1816 | GATTTAGCCACATTCAA | NC_045512.2 | 3′→5′ | 12215 | 12231 |
| 1817 | AGATTTAGCCACATTCA | NC_045512.2 | 3′→5′ | 12216 | 12232 |
| 1818 | CAGATTTAGCCACATTC | NC_045512.2 | 3′→5′ | 12217 | 12233 |
| 1819 | TCAGATTTAGCCACATT | NC_045512.2 | 3′→5′ | 12218 | 12234 |
| 1820 | GCCATCTTTTCCAACTT | NC_045512.2 | 3′→5′ | 12263 | 12279 |
| 1821 | TACATTTGGGTCATAGC | NC_045512.2 | 3′→5′ | 12287 | 12303 |
| 1822 | ACTTTTGCCCTCTTGTC | NC_045512.2 | 3′→5′ | 12323 | 12339 |
| 1823 | TTGTTGATAATGTTGTT | NC_045512.2 | 3′→5′ | 12401 | 12417 |
| 1824 | ATTGTTGATAATGTTGT | NC_045512.2 | 3′→5′ | 12402 | 12418 |
| 1825 | CATTGTTGATAATGTTG | NC_045512.2 | 3′→5′ | 12403 | 12419 |
| 1826 | GCATTGTTGATAATGTT | NC_045512.2 | 3′→5′ | 12404 | 12420 |
| 1827 | GACATCTGTCGTAGTGC | NC_045512.2 | 3′→5′ | 12707 | 12723 |
| 1828 | ACAAACCTACCTCCCTT | NC_045512.2 | 3′→5′ | 12791 | 12807 |
| 1829 | GGGAATCTAGCCCATTT | NC_045512.2 | 3′→5′ | 12839 | 12855 |
| 1830 | AGGGAATCTAGCCCATT | NC_045512.2 | 3′→5′ | 12840 | 12856 |
| 1831 | TAGGGAATCTAGCCCAT | NC_045512.2 | 3′→5′ | 12841 | 12857 |
| 1832 | TTAGGGAATCTAGCCCA | NC_045512.2 | 3′→5′ | 12842 | 12858 |
| 1833 | CTTAGGGAATCTAGCCC | NC_045512.2 | 3′→5′ | 12843 | 12859 |
| 1834 | TCTTAGGGAATCTAGCC | NC_045512.2 | 3′→5′ | 12844 | 12860 |
| 1835 | CTCTTAGGGAATCTAGC | NC_045512.2 | 3′→5′ | 12845 | 12861 |
| 1836 | ACTCTTAGGGAATCTAG | NC_045512.2 | 3′→5′ | 12846 | 12862 |
| 1837 | CACTCTTAGGGAATCTA | NC_045512.2 | 3′→5′ | 12847 | 12863 |
| 1838 | TCACTCTTAGGGAATCT | NC_045512.2 | 3′→5′ | 12848 | 12864 |
| 1839 | ATCACTCTTAGGGAATC | NC_045512.2 | 3′→5′ | 12849 | 12865 |
| 1840 | CATCACTCTTAGGGAAT | NC_045512.2 | 3′→5′ | 12850 | 12866 |
| 1841 | CCATCACTCTTAGGGAA | NC_045512.2 | 3′→5′ | 12851 | 12867 |
| 1842 | AGGTGGTTCCAGTTCTG | NC_045512.2 | 3′→5′ | 12885 | 12901 |
| 1843 | AAGGTGGTTCCAGTTCT | NC_045512.2 | 3′→5′ | 12886 | 12902 |
| 1844 | CAAGGTGGTTCCAGTTC | NC_045512.2 | 3′→5′ | 12887 | 12903 |
| 1845 | ACAAGGTGGTTCCAGTT | NC_045512.2 | 3′→5′ | 12888 | 12904 |
| 1846 | TACAAGGTGGTTCCAGT | NC_045512.2 | 3′→5′ | 12889 | 12905 |
| 1847 | CTACAAGGTGGTTCCAG | NC_045512.2 | 3′→5′ | 12890 | 12906 |
| 1848 | CCTACAAGGTGGTTCCA | NC_045512.2 | 3′→5′ | 12891 | 12907 |
| 1849 | ACCTACAAGGTGGTTCC | NC_045512.2 | 3′→5′ | 12892 | 12908 |
| 1850 | AACCTACAAGGTGGTTC | NC_045512.2 | 3′→5′ | 12893 | 12909 |
| 1851 | AAACCTACAAGGTGGTT | NC_045512.2 | 3′→5′ | 12894 | 12910 |
| 1852 | CAAACCTACAAGGTGGT | NC_045512.2 | 3′→5′ | 12895 | 12911 |
| 1853 | ACAAACCTACAAGGTGG | NC_045512.2 | 3′→5′ | 12896 | 12912 |
| 1854 | AACAAACCTACAAGGTG | NC_045512.2 | 3′→5′ | 12897 | 12913 |
| 1855 | TAACAAACCTACAAGGT | NC_045512.2 | 3′→5′ | 12898 | 12914 |
| 1856 | GTAACAAACCTACAAGG | NC_045512.2 | 3′→5′ | 12899 | 12915 |
| 1857 | TGTAACAAACCTACAAG | NC_045512.2 | 3′→5′ | 12900 | 12916 |
| 1858 | CTGTAACAAACCTACAA | NC_045512.2 | 3′→5′ | 12901 | 12917 |
| 1859 | TCTGTAACAAACCTACA | NC_045512.2 | 3′→5′ | 12902 | 12918 |
| 1860 | GTCTGTAACAAACCTAC | NC_045512.2 | 3′→5′ | 12903 | 12919 |
| 1861 | TGTCTGTAACAAACCTA | NC_045512.2 | 3′→5′ | 12904 | 12920 |
| 1862 | GTGTCTGTAACAAACCT | NC_045512.2 | 3′→5′ | 12905 | 12921 |
| 1863 | TGTGTCTGTAACAAACC | NC_045512.2 | 3′→5′ | 12906 | 12922 |
| 1864 | GTGTGTCTGTAACAAAC | NC_045512.2 | 3′→5′ | 12907 | 12923 |
| 1865 | GGTGTGTCTGTAACAAA | NC_045512.2 | 3′→5′ | 12908 | 12924 |
| 1866 | TCTATTTAGGTTGTTTA | NC_045512.2 | 3′→5′ | 12966 | 12982 |
| 1867 | CTCTATTTAGGTTGTTT | NC_045512.2 | 3′→5′ | 12967 | 12983 |
| 1868 | CCTCTATTTAGGTTGTT | NC_045512.2 | 3′→5′ | 12968 | 12984 |
| 1869 | ACCTCTATTTAGGTTGT | NC_045512.2 | 3′→5′ | 12969 | 12985 |
| 1870 | TACCTCTATTTAGGTTG | NC_045512.2 | 3′→5′ | 12970 | 12986 |
| 1871 | ATACCTCTATTTAGGTT | NC_045512.2 | 3′→5′ | 12971 | 12987 |
| 1872 | CATACCTCTATTTAGGT | NC_045512.2 | 3′→5′ | 12972 | 12988 |
| 1873 | CCATACCTCTATTTAGG | NC_045512.2 | 3′→5′ | 12973 | 12989 |
| 1874 | ACCATACCTCTATTTAG | NC_045512.2 | 3′→5′ | 12974 | 12990 |
| 1875 | ACAGTTGAATTGGCAGG | NC_045512.2 | 3′→5′ | 13046 | 13062 |
| 1876 | TGTGTACACAACATCTT | NC_045512.2 | 3′→5′ | 13151 | 13167 |
| 1877 | GTGTGTACACAACATCT | NC_045512.2 | 3′→5′ | 13152 | 13168 |
| 1878 | TGTGTGTACACAACATC | NC_045512.2 | 3′→5′ | 13153 | 13169 |
| 1879 | GTGTGTGTACACAACAT | NC_045512.2 | 3′→5′ | 13154 | 13170 |
| 1880 | AGTGTGTGTACACAACA | NC_045512.2 | 3′→5′ | 13155 | 13171 |
| 1881 | CAGTGTGTGTACACAAC | NC_045512.2 | 3′→5′ | 13156 | 13172 |
| 1882 | CCAGTGTGTGTACACAA | NC_045512.2 | 3′→5′ | 13157 | 13173 |
| 1883 | ACCAGTGTGTGTACACA | NC_045512.2 | 3′→5′ | 13158 | 13174 |
| 1884 | TACCAGTGTGTGTACAC | NC_045512.2 | 3′→5′ | 13159 | 13175 |
| 1885 | GTACCAGTGTGTGTACA | NC_045512.2 | 3′→5′ | 13160 | 13176 |
| 1886 | GGGTCATTAGCACAAGT | NC_045512.2 | 3′→5′ | 13328 | 13344 |
| 1887 | CGGTACAGACTGTGTTT | NC_045512.2 | 3′→5′ | 13363 | 13379 |
| 1888 | ACGGTACAGACTGTGTT | NC_045512.2 | 3′→5′ | 13364 | 13380 |
| 1889 | GACGGTACAGACTGTGT | NC_045512.2 | 3′→5′ | 13365 | 13381 |
| 1890 | AGACGGTACAGACTGTG | NC_045512.2 | 3′→5′ | 13366 | 13382 |
| 1891 | CAGACGGTACAGACTGT | NC_045512.2 | 3′→5′ | 13367 | 13383 |
| 1892 | GCAGACGGTACAGACTG | NC_045512.2 | 3′→5′ | 13368 | 13384 |
| 1893 | CGCAGACGGTACAGACT | NC_045512.2 | 3′→5′ | 13369 | 13385 |
| 1894 | CCGCAGACGGTACAGAC | NC_045512.2 | 3′→5′ | 13370 | 13386 |
| 1895 | CCATAACCTTTCCACAT | NC_045512.2 | 3′→5′ | 13388 | 13404 |
| 1896 | GCCATAACCTTTCCACA | NC_045512.2 | 3′→5′ | 13389 | 13405 |
| 1897 | AGCCATAACCTTTCCAC | NC_045512.2 | 3′→5′ | 13390 | 13406 |
| 1898 | CAGCCATAACCTTTCCA | NC_045512.2 | 3′→5′ | 13391 | 13407 |
| 1899 | ACAGCCATAACCTTTCC | NC_045512.2 | 3′→5′ | 13392 | 13408 |
| 1900 | TACAGCCATAACCTTTC | NC_045512.2 | 3′→5′ | 13393 | 13409 |
| 1901 | CTACAGCCATAACCTTT | NC_045512.2 | 3′→5′ | 13394 | 13410 |
| 1902 | ACTACAGCCATAACCTT | NC_045512.2 | 3′→5′ | 13395 | 13411 |
| 1903 | AACTACAGCCATAACCT | NC_045512.2 | 3′→5′ | 13396 | 13412 |
| 1904 | CAACTACAGCCATAACC | NC_045512.2 | 3′→5′ | 13397 | 13413 |
| 1905 | ACAACTACAGCCATAAC | NC_045512.2 | 3′→5′ | 13398 | 13414 |
| 1906 | CACAACTACAGCCATAA | NC_045512.2 | 3′→5′ | 13399 | 13415 |
| 1907 | TCACAACTACAGCCATA | NC_045512.2 | 3′→5′ | 13400 | 13416 |
| 1908 | AAACCCGTTTAAAAACG | NC_045512.2 | 3′→5′ | 13458 | 13474 |
| 1909 | CAAACCCGTTTAAAAAC | NC_045512.2 | 3′→5′ | 13459 | 13475 |
| 1910 | GCAAACCCGTTTAAAAA | NC_045512.2 | 3′→5′ | 13460 | 13476 |
| 1911 | CGCAAACCCGTTTAAAA | NC_045512.2 | 3′→5′ | 13461 | 13477 |
| 1912 | CCGCAAACCCGTTTAAA | NC_045512.2 | 3′→5′ | 13462 | 13478 |
| 1913 | ACCGCAAACCCGTTTAA | NC_045512.2 | 3′→5′ | 13463 | 13479 |
| 1914 | CACCGCAAACCCGTTTA | NC_045512.2 | 3′→5′ | 13464 | 13480 |
| 1915 | ACACCGCAAACCCGTTT | NC_045512.2 | 3′→5′ | 13465 | 13481 |
| 1916 | TACACCGCAAACCCGTT | NC_045512.2 | 3′→5′ | 13466 | 13482 |
| 1917 | TTACACCGCAAACCCGT | NC_045512.2 | 3′→5′ | 13467 | 13483 |
| 1918 | CTTACACCGCAAACCCG | NC_045512.2 | 3′→5′ | 13468 | 13484 |
| 1919 | ACTTACACCGCAAACCC | NC_045512.2 | 3′→5′ | 13469 | 13485 |
| 1920 | CACTTACACCGCAAACC | NC_045512.2 | 3′→5′ | 13470 | 13486 |
| 1921 | GCACTTACACCGCAAAC | NC_045512.2 | 3′→5′ | 13471 | 13487 |
| 1922 | TGCACTTACACCGCAAA | NC_045512.2 | 3′→5′ | 13472 | 13488 |
| 1923 | CTGCACTTACACCGCAA | NC_045512.2 | 3′→5′ | 13473 | 13489 |
| 1924 | GCTGCACTTACACCGCA | NC_045512.2 | 3′→5′ | 13474 | 13490 |
| 1925 | GGCTGCACTTACACCGC | NC_045512.2 | 3′→5′ | 13475 | 13491 |
| 1926 | GGGCTGCACTTACACCG | NC_045512.2 | 3′→5′ | 13476 | 13492 |
| 1927 | CGGGCTGCACTTACACC | NC_045512.2 | 3′→5′ | 13477 | 13493 |
| 1928 | ACGGGCTGCACTTACAC | NC_045512.2 | 3′→5′ | 13478 | 13494 |
| 1929 | GACGGGCTGCACTTACA | NC_045512.2 | 3′→5′ | 13479 | 13495 |
| 1930 | AGACGGGCTGCACTTAC | NC_045512.2 | 3′→5′ | 13480 | 13496 |
| 1931 | AAGACGGGCTGCACTTA | NC_045512.2 | 3′→5′ | 13481 | 13497 |
| 1932 | TAAGACGGGCTGCACTT | NC_045512.2 | 3′→5′ | 13482 | 13498 |
| 1933 | GTAAGACGGGCTGCACT | NC_045512.2 | 3′→5′ | 13483 | 13499 |
| 1934 | TGTAAGACGGGCTGCAC | NC_045512.2 | 3′→5′ | 13484 | 13500 |
| 1935 | GTGTAAGACGGGCTGCA | NC_045512.2 | 3′→5′ | 13485 | 13501 |
| 1936 | GGTGTAAGACGGGCTGC | NC_045512.2 | 3′→5′ | 13486 | 13502 |
| 1937 | CGGTGTAAGACGGGCTG | NC_045512.2 | 3′→5′ | 13487 | 13503 |
| 1938 | ACGGTGTAAGACGGGCT | NC_045512.2 | 3′→5′ | 13488 | 13504 |
| 1939 | CACGGTGTAAGACGGGC | NC_045512.2 | 3′→5′ | 13489 | 13505 |
| 1940 | GCACGGTGTAAGACGGG | NC_045512.2 | 3′→5′ | 13490 | 13506 |
| 1941 | CGCACGGTGTAAGACGG | NC_045512.2 | 3′→5′ | 13491 | 13507 |
| 1942 | CCGCACGGTGTAAGACG | NC_045512.2 | 3′→5′ | 13492 | 13508 |
| 1943 | GCCGCACGGTGTAAGAC | NC_045512.2 | 3′→5′ | 13493 | 13509 |
| 1944 | TGCCGCACGGTGTAAGA | NC_045512.2 | 3′→5′ | 13494 | 13510 |
| 1945 | GTGCCGCACGGTGTAAG | NC_045512.2 | 3′→5′ | 13495 | 13511 |
| 1946 | TGTGCCGCACGGTGTAA | NC_045512.2 | 3′→5′ | 13496 | 13512 |
| 1947 | CTGTGCCGCACGGTGTA | NC_045512.2 | 3′→5′ | 13497 | 13513 |
| 1948 | CCTGTGCCGCACGGTGT | NC_045512.2 | 3′→5′ | 13498 | 13514 |
| 1949 | GCCTGTGCCGCACGGTG | NC_045512.2 | 3′→5′ | 13499 | 13515 |
| 1950 | TGCCTGTGCCGCACGGT | NC_045512.2 | 3′→5′ | 13500 | 13516 |
| 1951 | GTGCCTGTGCCGCACGG | NC_045512.2 | 3′→5′ | 13501 | 13517 |
| 1952 | AGTGCCTGTGCCGCACG | NC_045512.2 | 3′→5′ | 13502 | 13518 |
| 1953 | TAGTGCCTGTGCCGCAC | NC_045512.2 | 3′→5′ | 13503 | 13519 |
| 1954 | CTAGTGCCTGTGCCGCA | NC_045512.2 | 3′→5′ | 13504 | 13520 |
| 1955 | CAATTAGTTTTTAGGAA | NC_045512.2 | 3′→5′ | 13582 | 13598 |
| 1956 | TCTTCATGTTGGTAGTT | NC_045512.2 | 3′→5′ | 13675 | 13691 |
| 1957 | TGTGGTACCATGTCACC | NC_045512.2 | 3′→5′ | 13762 | 13778 |
| 1958 | ATGTGGTACCATGTCAC | NC_045512.2 | 3′→5′ | 13763 | 13779 |
| 1959 | TATGTGGTACCATGTCA | NC_045512.2 | 3′→5′ | 13764 | 13780 |
| 1960 | ATATGTGGTACCATGTC | NC_045512.2 | 3′→5′ | 13765 | 13781 |
| 1961 | TATATGTGGTACCATGT | NC_045512.2 | 3′→5′ | 13766 | 13782 |
| 1962 | ATATATGTGGTACCATG | NC_045512.2 | 3′→5′ | 13767 | 13783 |
| 1963 | GATATATGTGGTACCAT | NC_045512.2 | 3′→5′ | 13768 | 13784 |
| 1964 | TGATATATGTGGTACCA | NC_045512.2 | 3′→5′ | 13769 | 13785 |
| 1965 | GTGATATATGTGGTACC | NC_045512.2 | 3′→5′ | 13770 | 13786 |
| 1966 | CGTGATATATGTGGTAC | NC_045512.2 | 3′→5′ | 13771 | 13787 |
| 1967 | ACGTGATATATGTGGTA | NC_045512.2 | 3′→5′ | 13772 | 13788 |
| 1968 | GACGTGATATATGTGGT | NC_045512.2 | 3′→5′ | 13773 | 13789 |
| 1969 | TGACGTGATATATGTGG | NC_045512.2 | 3′→5′ | 13774 | 13790 |
| 1970 | GCCATTGTGTATTTAGT | NC_045512.2 | 3′→5′ | 13798 | 13814 |
| 1971 | TATTTAAAATAACGGTC | NC_045512.2 | 3′→5′ | 14290 | 14306 |
| 1972 | ATATTTAAAATAACGGT | NC_045512.2 | 3′→5′ | 14291 | 14307 |
| 1973 | AATATTTAAAATAACGG | NC_045512.2 | 3′→5′ | 14292 | 14308 |
| 1974 | CAATATTTAAAATAACG | NC_045512.2 | 3′→5′ | 14293 | 14309 |
| 1975 | CCAATATTTAAAATAAC | NC_045512.2 | 3′→5′ | 14294 | 14310 |
| 1976 | CCCAATATTTAAAATAA | NC_045512.2 | 3′→5′ | 14295 | 14311 |
| 1977 | TCCCAATATTTAAAATA | NC_045512.2 | 3′→5′ | 14296 | 14312 |
| 1978 | CCAAAACTTGTAGGTGG | NC_045512.2 | 3′→5′ | 14404 | 14420 |
| 1979 | TCCAAAACTTGTAGGTG | NC_045512.2 | 3′→5′ | 14405 | 14421 |
| 1980 | GTCCAAAACTTGTAGGT | NC_045512.2 | 3′→5′ | 14406 | 14422 |
| 1981 | GGTCCAAAACTTGTAGG | NC_045512.2 | 3′→5′ | 14407 | 14423 |
| 1982 | TGGTCCAAAACTTGTAG | NC_045512.2 | 3′→5′ | 14408 | 14424 |
| 1983 | GTGGTCCAAAACTTGTA | NC_045512.2 | 3′→5′ | 14409 | 14425 |
| 1984 | AGTGGTCCAAAACTTGT | NC_045512.2 | 3′→5′ | 14410 | 14426 |
| 1985 | TAGTGGTCCAAAACTTG | NC_045512.2 | 3′→5′ | 14411 | 14427 |
| 1986 | CTAGTGGTCCAAAACTT | NC_045512.2 | 3′→5′ | 14412 | 14428 |
| 1987 | ACTAGTGGTCCAAAACT | NC_045512.2 | 3′→5′ | 14413 | 14429 |
| 1988 | ACATCCTGATTATGTAC | NC_045512.2 | 3′→5′ | 14500 | 14516 |
| 1989 | TACATCCTGATTATGTA | NC_045512.2 | 3′→5′ | 14501 | 14517 |
| 1990 | TTACATCCTGATTATGT | NC_045512.2 | 3′→5′ | 14502 | 14518 |
| 1991 | TTTACATCCTGATTATG | NC_045512.2 | 3′→5′ | 14503 | 14519 |
| 1992 | GTTTACATCCTGATTAT | NC_045512.2 | 3′→5′ | 14504 | 14520 |
| 1993 | AGTTTACATCCTGATTA | NC_045512.2 | 3′→5′ | 14505 | 14521 |
| 1994 | AAGTTTACATCCTGATT | NC_045512.2 | 3′→5′ | 14506 | 14522 |
| 1995 | TAAGTTTACATCCTGAT | NC_045512.2 | 3′→5′ | 14507 | 14523 |
| 1996 | GTAAGTTTACATCCTGA | NC_045512.2 | 3′→5′ | 14508 | 14524 |
| 1997 | TGTAAGTTTACATCCTG | NC_045512.2 | 3′→5′ | 14509 | 14525 |
| 1998 | ATGTAAGTTTACATCCT | NC_045512.2 | 3′→5′ | 14510 | 14526 |
| 1999 | TATGTAAGTTTACATCC | NC_045512.2 | 3′→5′ | 14511 | 14527 |
| 2000 | CTATGTAAGTTTACATC | NC_045512.2 | 3′→5′ | 14512 | 14528 |
| 2001 | GCTATGTAAGTTTACAT | NC_045512.2 | 3′→5′ | 14513 | 14529 |
| 2002 | AGCTATGTAAGTTTACA | NC_045512.2 | 3′→5′ | 14514 | 14530 |
| 2003 | GAGCTATGTAAGTTTAC | NC_045512.2 | 3′→5′ | 14515 | 14531 |
| 2004 | GTAGTGCGTTTATCTAG | NC_045512.2 | 3′→5′ | 14605 | 14621 |
| 2005 | GCAGCTACTGAAAAGCA | NC_045512.2 | 3′→5′ | 14623 | 14639 |
| 2006 | TGCAGCTACTGAAAAGC | NC_045512.2 | 3′→5′ | 14624 | 14640 |
| 2007 | GTGCAGCTACTGAAAAG | NC_045512.2 | 3′→5′ | 14625 | 14641 |
| 2008 | AGTGCAGCTACTGAAAA | NC_045512.2 | 3′→5′ | 14626 | 14642 |
| 2009 | GTTTGAAAAGCAACATT | NC_045512.2 | 3′→5′ | 14650 | 14666 |
| 2010 | AGTTTGAAAAGCAACAT | NC_045512.2 | 3′→5′ | 14651 | 14667 |
| 2011 | CAGTTTGAAAAGCAACA | NC_045512.2 | 3′→5′ | 14652 | 14668 |
| 2012 | ACAGTTTGAAAAGCAAC | NC_045512.2 | 3′→5′ | 14653 | 14669 |
| 2013 | GACAGTTTGAAAAGCAA | NC_045512.2 | 3′→5′ | 14654 | 14670 |
| 2014 | TGACAGTTTGAAAAGCA | NC_045512.2 | 3′→5′ | 14655 | 14671 |
| 2015 | TTGACAGTTTGAAAAGC | NC_045512.2 | 3′→5′ | 14656 | 14672 |
| 2016 | TTTGACAGTTTGAAAAG | NC_045512.2 | 3′→5′ | 14657 | 14673 |
| 2017 | GTTTGACAGTTTGAAAA | NC_045512.2 | 3′→5′ | 14658 | 14674 |
| 2018 | GGTTTGACAGTTTGAAA | NC_045512.2 | 3′→5′ | 14659 | 14675 |
| 2019 | GGGTTTGACAGTTTGAA | NC_045512.2 | 3′→5′ | 14660 | 14676 |
| 2020 | CGGGTTTGACAGTTTGA | NC_045512.2 | 3′→5′ | 14661 | 14677 |
| 2021 | CCGGGTTTGACAGTTTG | NC_045512.2 | 3′→5′ | 14662 | 14678 |
| 2022 | ACCGGGTTTGACAGTTT | NC_045512.2 | 3′→5′ | 14663 | 14679 |
| 2023 | TACCGGGTTTGACAGTT | NC_045512.2 | 3′→5′ | 14664 | 14680 |
| 2024 | TTACCGGGTTTGACAGT | NC_045512.2 | 3′→5′ | 14665 | 14681 |
| 2025 | ATTACCGGGTTTGACAG | NC_045512.2 | 3′→5′ | 14666 | 14682 |
| 2026 | AATTACCGGGTTTGACA | NC_045512.2 | 3′→5′ | 14667 | 14683 |
| 2027 | AAATTACCGGGTTTGAC | NC_045512.2 | 3′→5′ | 14668 | 14684 |
| 2028 | AAAATTACCGGGTTTGA | NC_045512.2 | 3′→5′ | 14669 | 14685 |
| 2029 | TAAAATTACCGGGTTTG | NC_045512.2 | 3′→5′ | 14670 | 14686 |
| 2030 | TTAAAATTACCGGGTTT | NC_045512.2 | 3′→5′ | 14671 | 14687 |
| 2031 | GACACAGCAAAGTCATA | NC_045512.2 | 3′→5′ | 14698 | 14714 |
| 2032 | AGACACAGCAAAGTCAT | NC_045512.2 | 3′→5′ | 14699 | 14715 |
| 2033 | TAGACACAGCAAAGTCA | NC_045512.2 | 3′→5′ | 14700 | 14716 |
| 2034 | TTAGACACAGCAAAGTC | NC_045512.2 | 3′→5′ | 14701 | 14717 |
| 2035 | CTTCCTTCCTTAAAGAA | NC_045512.2 | 3′→5′ | 14722 | 14738 |
| 2036 | ACTTCCTTCCTTAAAGA | NC_045512.2 | 3′→5′ | 14723 | 14739 |
| 2037 | AACTTCCTTCCTTAAAG | NC_045512.2 | 3′→5′ | 14724 | 14740 |
| 2038 | GAACTTCCTTCCTTAAA | NC_045512.2 | 3′→5′ | 14725 | 14741 |
| 2039 | AGAACTTCCTTCCTTAA | NC_045512.2 | 3′→5′ | 14726 | 14742 |
| 2040 | CAGAACTTCCTTCCTTA | NC_045512.2 | 3′→5′ | 14727 | 14743 |
| 2041 | ACAGAACTTCCTTCCTT | NC_045512.2 | 3′→5′ | 14728 | 14744 |
| 2042 | AACAGAACTTCCTTCCT | NC_045512.2 | 3′→5′ | 14729 | 14745 |
| 2043 | CAACAGAACTTCCTTCC | NC_045512.2 | 3′→5′ | 14730 | 14746 |
| 2044 | TCAACAGAACTTCCTTC | NC_045512.2 | 3′→5′ | 14731 | 14747 |
| 2045 | TTCAACAGAACTTCCTT | NC_045512.2 | 3′→5′ | 14732 | 14748 |
| 2046 | AAAGAAGAAGTGTTTTA | NC_045512.2 | 3′→5′ | 14750 | 14766 |
| 2047 | CAAAGAAGAAGTGTTTT | NC_045512.2 | 3′→5′ | 14751 | 14767 |
| 2048 | GCAAAGAAGAAGTGTTT | NC_045512.2 | 3′→5′ | 14752 | 14768 |
| 2049 | AGCAAAGAAGAAGTGTT | NC_045512.2 | 3′→5′ | 14753 | 14769 |
| 2050 | GAGCAAAGAAGAAGTGT | NC_045512.2 | 3′→5′ | 14754 | 14770 |
| 2051 | TGAGCAAAGAAGAAGTG | NC_045512.2 | 3′→5′ | 14755 | 14771 |
| 2052 | CTGAGCAAAGAAGAAGT | NC_045512.2 | 3′→5′ | 14756 | 14772 |
| 2053 | CCTGAGCAAAGAAGAAG | NC_045512.2 | 3′→5′ | 14757 | 14773 |
| 2054 | TCCTGAGCAAAGAAGAA | NC_045512.2 | 3′→5′ | 14758 | 14774 |
| 2055 | ATCCTGAGCAAAGAAGA | NC_045512.2 | 3′→5′ | 14759 | 14775 |
| 2056 | CATCCTGAGCAAAGAAG | NC_045512.2 | 3′→5′ | 14760 | 14776 |
| 2057 | CCATCCTGAGCAAAGAA | NC_045512.2 | 3′→5′ | 14761 | 14777 |
| 2058 | ATATCACACATTGTTGG | NC_045512.2 | 3′→5′ | 14821 | 14837 |
| 2059 | GATATCACACATTGTTG | NC_045512.2 | 3′→5′ | 14822 | 14838 |
| 2060 | TGATATCACACATTGTT | NC_045512.2 | 3′→5′ | 14823 | 14839 |
| 2061 | CTGATATCACACATTGT | NC_045512.2 | 3′→5′ | 14824 | 14840 |
| 2062 | TCTGATATCACACATTG | NC_045512.2 | 3′→5′ | 14825 | 14841 |
| 2063 | GTCTGATATCACACATT | NC_045512.2 | 3′→5′ | 14826 | 14842 |
| 2064 | TGTCTGATATCACACAT | NC_045512.2 | 3′→5′ | 14827 | 14843 |
| 2065 | TTGTCTGATATCACACA | NC_045512.2 | 3′→5′ | 14828 | 14844 |
| 2066 | GTTGTCTGATATCACAC | NC_045512.2 | 3′→5′ | 14829 | 14845 |
| 2067 | AGTTGTCTGATATCACA | NC_045512.2 | 3′→5′ | 14830 | 14846 |
| 2068 | TCAACAACTTCAACTAC | NC_045512.2 | 3′→5′ | 14854 | 14870 |
| 2069 | ATCAACAACTTCAACTA | NC_045512.2 | 3′→5′ | 14855 | 14871 |
| 2070 | TATCAACAACTTCAACT | NC_045512.2 | 3′→5′ | 14856 | 14872 |
| 2071 | TTATCAACAACTTCAAC | NC_045512.2 | 3′→5′ | 14857 | 14873 |
| 2072 | TCGTAACAATCAAAGTA | NC_045512.2 | 3′→5′ | 14875 | 14891 |
| 2073 | ATCGTAACAATCAAAGT | NC_045512.2 | 3′→5′ | 14876 | 14892 |
| 2074 | CATCGTAACAATCAAAG | NC_045512.2 | 3′→5′ | 14877 | 14893 |
| 2075 | CCATCGTAACAATCAAA | NC_045512.2 | 3′→5′ | 14878 | 14894 |
| 2076 | ACCATCGTAACAATCAA | NC_045512.2 | 3′→5′ | 14879 | 14895 |
| 2077 | CACCATCGTAACAATCA | NC_045512.2 | 3′→5′ | 14880 | 14896 |
| 2078 | CCACCATCGTAACAATC | NC_045512.2 | 3′→5′ | 14881 | 14897 |
| 2079 | GCCACCATCGTAACAAT | NC_045512.2 | 3′→5′ | 14882 | 14898 |
| 2080 | AGCCACCATCGTAACAA | NC_045512.2 | 3′→5′ | 14883 | 14899 |
| 2081 | CAGCCACCATCGTAACA | NC_045512.2 | 3′→5′ | 14884 | 14900 |
| 2082 | ACAGCCACCATCGTAAC | NC_045512.2 | 3′→5′ | 14885 | 14901 |
| 2083 | TACAGCCACCATCGTAA | NC_045512.2 | 3′→5′ | 14886 | 14902 |
| 2084 | ATACAGCCACCATCGTA | NC_045512.2 | 3′→5′ | 14887 | 14903 |
| 2085 | CTAGCCTTACCCCATTT | NC_045512.2 | 3′→5′ | 14962 | 14978 |
| 2086 | TCTAGCCTTACCCCATT | NC_045512.2 | 3′→5′ | 14963 | 14979 |
| 2087 | GTCTAGCCTTACCCCAT | NC_045512.2 | 3′→5′ | 14964 | 14980 |
| 2088 | AGTCTAGCCTTACCCCA | NC_045512.2 | 3′→5′ | 14965 | 14981 |
| 2089 | AAGTCTAGCCTTACCCC | NC_045512.2 | 3′→5′ | 14966 | 14982 |
| 2090 | AAAGTCTAGCCTTACCC | NC_045512.2 | 3′→5′ | 14967 | 14983 |
| 2091 | TAAAGTCTAGCCTTACC | NC_045512.2 | 3′→5′ | 14968 | 14984 |
| 2092 | ATAAAGTCTAGCCTTAC | NC_045512.2 | 3′→5′ | 14969 | 14985 |
| 2093 | AATAAAGTCTAGCCTTA | NC_045512.2 | 3′→5′ | 14970 | 14986 |
| 2094 | TAATAAAGTCTAGCCTT | NC_045512.2 | 3′→5′ | 14971 | 14987 |
| 2095 | ATAATAAAGTCTAGCCT | NC_045512.2 | 3′→5′ | 14972 | 14988 |
| 2096 | CATAATAAAGTCTAGCC | NC_045512.2 | 3′→5′ | 14973 | 14989 |
| 2097 | TCATAATAAAGTCTAGC | NC_045512.2 | 3′→5′ | 14974 | 14990 |
| 2098 | TCCTCATAACTCATTGA | NC_045512.2 | 3′→5′ | 14992 | 15008 |
| 2099 | ATCCTCATAACTCATTG | NC_045512.2 | 3′→5′ | 14993 | 15009 |
| 2100 | GATCCTCATAACTCATT | NC_045512.2 | 3′→5′ | 14994 | 15010 |
| 2101 | TGATCCTCATAACTCAT | NC_045512.2 | 3′→5′ | 14995 | 15011 |
| 2102 | TTGATCCTCATAACTCA | NC_045512.2 | 3′→5′ | 14996 | 15012 |
| 2103 | CTTGATCCTCATAACTC | NC_045512.2 | 3′→5′ | 14997 | 15013 |
| 2104 | TCTTGATCCTCATAACT | NC_045512.2 | 3′→5′ | 14998 | 15014 |
| 2105 | ATCTTGATCCTCATAAC | NC_045512.2 | 3′→5′ | 14999 | 15015 |
| 2106 | CATCTTGATCCTCATAA | NC_045512.2 | 3′→5′ | 15000 | 15016 |
| 2107 | GCATCTTGATCCTCATA | NC_045512.2 | 3′→5′ | 15001 | 15017 |
| 2108 | TGCATCTTGATCCTCAT | NC_045512.2 | 3′→5′ | 15002 | 15018 |
| 2109 | GTGCATCTTGATCCTCA | NC_045512.2 | 3′→5′ | 15003 | 15019 |
| 2110 | AGTGCATCTTGATCCTC | NC_045512.2 | 3′→5′ | 15004 | 15020 |
| 2111 | GTAGGGATGACATTACG | NC_045512.2 | 3′→5′ | 15037 | 15053 |
| 2112 | AGATTCATTTGAGTTAT | NC_045512.2 | 3′→5′ | 15055 | 15071 |
| 2113 | AAGATTCATTTGAGTTA | NC_045512.2 | 3′→5′ | 15056 | 15072 |
| 2114 | TAAGATTCATTTGAGTT | NC_045512.2 | 3′→5′ | 15057 | 15073 |
| 2115 | TTAAGATTCATTTGAGT | NC_045512.2 | 3′→5′ | 15058 | 15074 |
| 2116 | CTTAAGATTCATTTGAG | NC_045512.2 | 3′→5′ | 15059 | 15075 |
| 2117 | ACTTAAGATTCATTTGA | NC_045512.2 | 3′→5′ | 15060 | 15076 |
| 2118 | TACTTAAGATTCATTTG | NC_045512.2 | 3′→5′ | 15061 | 15077 |
| 2119 | ATACTTAAGATTCATTT | NC_045512.2 | 3′→5′ | 15062 | 15078 |
| 2120 | CATACTTAAGATTCATT | NC_045512.2 | 3′→5′ | 15063 | 15079 |
| 2121 | GCATACTTAAGATTCAT | NC_045512.2 | 3′→5′ | 15064 | 15080 |
| 2122 | GGCATACTTAAGATTCA | NC_045512.2 | 3′→5′ | 15065 | 15081 |
| 2123 | TGGCATACTTAAGATTC | NC_045512.2 | 3′→5′ | 15066 | 15082 |
| 2124 | ATGGCATACTTAAGATT | NC_045512.2 | 3′→5′ | 15067 | 15083 |
| 2125 | AATGGCATACTTAAGAT | NC_045512.2 | 3′→5′ | 15068 | 15084 |
| 2126 | TAATGGCATACTTAAGA | NC_045512.2 | 3′→5′ | 15069 | 15085 |
| 2127 | CTAATGGCATACTTAAG | NC_045512.2 | 3′→5′ | 15070 | 15086 |
| 2128 | ACTAATGGCATACTTAA | NC_045512.2 | 3′→5′ | 15071 | 15087 |
| 2129 | CACTAATGGCATACTTA | NC_045512.2 | 3′→5′ | 15072 | 15088 |
| 2130 | GCACTAATGGCATACTT | NC_045512.2 | 3′→5′ | 15073 | 15089 |
| 2131 | TGCACTAATGGCATACT | NC_045512.2 | 3′→5′ | 15074 | 15090 |
| 2132 | TTGCACTAATGGCATAC | NC_045512.2 | 3′→5′ | 15075 | 15091 |
| 2133 | TTTGCACTAATGGCATA | NC_045512.2 | 3′→5′ | 15076 | 15092 |
| 2134 | CTTTGCACTAATGGCAT | NC_045512.2 | 3′→5′ | 15077 | 15093 |
| 2135 | TCTTTGCACTAATGGCA | NC_045512.2 | 3′→5′ | 15078 | 15094 |
| 2136 | TTCTTTGCACTAATGGC | NC_045512.2 | 3′→5′ | 15079 | 15095 |
| 2137 | ATTCTTTGCACTAATGG | NC_045512.2 | 3′→5′ | 15080 | 15096 |
| 2138 | TATTCTTTGCACTAATG | NC_045512.2 | 3′→5′ | 15081 | 15097 |
| 2139 | CTATTCTTTGCACTAAT | NC_045512.2 | 3′→5′ | 15082 | 15098 |
| 2140 | TCTATTCTTTGCACTAA | NC_045512.2 | 3′→5′ | 15083 | 15099 |
| 2141 | CTCTATTCTTTGCACTA | NC_045512.2 | 3′→5′ | 15084 | 15100 |
| 2142 | GCTCTATTCTTTGCACT | NC_045512.2 | 3′→5′ | 15085 | 15101 |
| 2143 | AGCTCTATTCTTTGCAC | NC_045512.2 | 3′→5′ | 15086 | 15102 |
| 2144 | GAGCTCTATTCTTTGCA | NC_045512.2 | 3′→5′ | 15087 | 15103 |
| 2145 | CGAGCTCTATTCTTTGC | NC_045512.2 | 3′→5′ | 15088 | 15104 |
| 2146 | GCGAGCTCTATTCTTTG | NC_045512.2 | 3′→5′ | 15089 | 15105 |
| 2147 | TGCGAGCTCTATTCTTT | NC_045512.2 | 3′→5′ | 15090 | 15106 |
| 2148 | GTGCGAGCTCTATTCTT | NC_045512.2 | 3′→5′ | 15091 | 15107 |
| 2149 | GGTGCGAGCTCTATTCT | NC_045512.2 | 3′→5′ | 15092 | 15108 |
| 2150 | CGGTGCGAGCTCTATTC | NC_045512.2 | 3′→5′ | 15093 | 15109 |
| 2151 | ACGGTGCGAGCTCTATT | NC_045512.2 | 3′→5′ | 15094 | 15110 |
| 2152 | TACGGTGCGAGCTCTAT | NC_045512.2 | 3′→5′ | 15095 | 15111 |
| 2153 | CTACGGTGCGAGCTCTA | NC_045512.2 | 3′→5′ | 15096 | 15112 |
| 2154 | GCTACGGTGCGAGCTCT | NC_045512.2 | 3′→5′ | 15097 | 15113 |
| 2155 | AGCTACGGTGCGAGCTC | NC_045512.2 | 3′→5′ | 15098 | 15114 |
| 2156 | CAGCTACGGTGCGAGCT | NC_045512.2 | 3′→5′ | 15099 | 15115 |
| 2157 | CCAGCTACGGTGCGAGC | NC_045512.2 | 3′→5′ | 15100 | 15116 |
| 2158 | ACCAGCTACGGTGCGAG | NC_045512.2 | 3′→5′ | 15101 | 15117 |
| 2159 | CACCAGCTACGGTGCGA | NC_045512.2 | 3′→5′ | 15102 | 15118 |
| 2160 | ACACCAGCTACGGTGCG | NC_045512.2 | 3′→5′ | 15103 | 15119 |
| 2161 | GACACCAGCTACGGTGC | NC_045512.2 | 3′→5′ | 15104 | 15120 |
| 2162 | AGACACCAGCTACGGTG | NC_045512.2 | 3′→5′ | 15105 | 15121 |
| 2163 | GAGACACCAGCTACGGT | NC_045512.2 | 3′→5′ | 15106 | 15122 |
| 2164 | AGAGACACCAGCTACGG | NC_045512.2 | 3′→5′ | 15107 | 15123 |
| 2165 | TAGAGACACCAGCTACG | NC_045512.2 | 3′→5′ | 15108 | 15124 |
| 2166 | ATAGAGACACCAGCTAC | NC_045512.2 | 3′→5′ | 15109 | 15125 |
| 2167 | GATAGAGACACCAGCTA | NC_045512.2 | 3′→5′ | 15110 | 15126 |
| 2168 | AGATAGAGACACCAGCT | NC_045512.2 | 3′→5′ | 15111 | 15127 |
| 2169 | CAGATAGAGACACCAGC | NC_045512.2 | 3′→5′ | 15112 | 15128 |
| 2170 | ACAGATAGAGACACCAG | NC_045512.2 | 3′→5′ | 15113 | 15129 |
| 2171 | TACAGATAGAGACACCA | NC_045512.2 | 3′→5′ | 15114 | 15130 |
| 2172 | CTACAGATAGAGACACC | NC_045512.2 | 3′→5′ | 15115 | 15131 |
| 2173 | ACTACAGATAGAGACAC | NC_045512.2 | 3′→5′ | 15116 | 15132 |
| 2174 | TACTACAGATAGAGACA | NC_045512.2 | 3′→5′ | 15117 | 15133 |
| 2175 | GTACTACAGATAGAGAC | NC_045512.2 | 3′→5′ | 15118 | 15134 |
| 2176 | AGTACTACAGATAGAGA | NC_045512.2 | 3′→5′ | 15119 | 15135 |
| 2177 | TAGTACTACAGATAGAG | NC_045512.2 | 3′→5′ | 15120 | 15136 |
| 2178 | ATAGTACTACAGATAGA | NC_045512.2 | 3′→5′ | 15121 | 15137 |
| 2179 | CATAGTACTACAGATAG | NC_045512.2 | 3′→5′ | 15122 | 15138 |
| 2180 | TCATAGTACTACAGATA | NC_045512.2 | 3′→5′ | 15123 | 15139 |
| 2181 | GTCATAGTACTACAGAT | NC_045512.2 | 3′→5′ | 15124 | 15140 |
| 2182 | TGATGAAACTGTCTATT | NC_045512.2 | 3′→5′ | 15142 | 15158 |
| 2183 | CTAGTGGCGGCTATTGA | NC_045512.2 | 3′→5′ | 15172 | 15188 |
| 2184 | TCTAGTGGCGGCTATTG | NC_045512.2 | 3′→5′ | 15173 | 15189 |
| 2185 | CTCTAGTGGCGGCTATT | NC_045512.2 | 3′→5′ | 15174 | 15190 |
| 2186 | CCTCTAGTGGCGGCTAT | NC_045512.2 | 3′→5′ | 15175 | 15191 |
| 2187 | TCCTCTAGTGGCGGCTA | NC_045512.2 | 3′→5′ | 15176 | 15192 |
| 2188 | CTCCTCTAGTGGCGGCT | NC_045512.2 | 3′→5′ | 15177 | 15193 |
| 2189 | GCTCCTCTAGTGGCGGC | NC_045512.2 | 3′→5′ | 15178 | 15194 |
| 2190 | AGCTCCTCTAGTGGCGG | NC_045512.2 | 3′→5′ | 15179 | 15195 |
| 2191 | TAGCTCCTCTAGTGGCG | NC_045512.2 | 3′→5′ | 15180 | 15196 |
| 2192 | GTAGCTCCTCTAGTGGC | NC_045512.2 | 3′→5′ | 15181 | 15197 |
| 2193 | AGTAGCTCCTCTAGTGG | NC_045512.2 | 3′→5′ | 15182 | 15198 |
| 2194 | CAGTAGCTCCTCTAGTG | NC_045512.2 | 3′→5′ | 15183 | 15199 |
| 2195 | ACAGTAGCTCCTCTAGT | NC_045512.2 | 3′→5′ | 15184 | 15200 |
| 2196 | TTGCTTGTTCCAATTAC | NC_045512.2 | 3′→5′ | 15202 | 15218 |
| 2197 | TAAACAGTTTTTAACAT | NC_045512.2 | 3′→5′ | 15241 | 15257 |
| 2198 | TCCCAACCCATAAGGTG | NC_045512.2 | 3′→5′ | 15277 | 15293 |
| 2199 | ATGTTAGGCATGGCTCT | NC_045512.2 | 3′→5′ | 15310 | 15326 |
| 2200 | CATGTTAGGCATGGCTC | NC_045512.2 | 3′→5′ | 15311 | 15327 |
| 2201 | GCATGTTAGGCATGGCT | NC_045512.2 | 3′→5′ | 15312 | 15328 |
| 2202 | AGCATGTTAGGCATGGC | NC_045512.2 | 3′→5′ | 15313 | 15329 |
| 2203 | AAGCATGTTAGGCATGG | NC_045512.2 | 3′→5′ | 15314 | 15330 |
| 2204 | TAAGCATGTTAGGCATG | NC_045512.2 | 3′→5′ | 15315 | 15331 |
| 2205 | CTAAGCATGTTAGGCAT | NC_045512.2 | 3′→5′ | 15316 | 15332 |
| 2206 | TTGCGAGCAAGAACAAG | NC_045512.2 | 3′→5′ | 15346 | 15362 |
| 2207 | TTTGCGAGCAAGAACAA | NC_045512.2 | 3′→5′ | 15347 | 15363 |
| 2208 | GTTTGCGAGCAAGAACA | NC_045512.2 | 3′→5′ | 15348 | 15364 |
| 2209 | TGTTTGCGAGCAAGAAC | NC_045512.2 | 3′→5′ | 15349 | 15365 |
| 2210 | ATGTTTGCGAGCAAGAA | NC_045512.2 | 3′→5′ | 15350 | 15366 |
| 2211 | TATGTTTGCGAGCAAGA | NC_045512.2 | 3′→5′ | 15351 | 15367 |
| 2212 | CCGCCACACATGACCAT | NC_045512.2 | 3′→5′ | 15436 | 15452 |
| 2213 | TTAGCATAAGCAGTTGT | NC_045512.2 | 3′→5′ | 15496 | 15512 |
| 2214 | ATTAGCATAAGCAGTTG | NC_045512.2 | 3′→5′ | 15497 | 15513 |
| 2215 | TATTAGCATAAGCAGTT | NC_045512.2 | 3′→5′ | 15498 | 15514 |
| 2216 | CTATTAGCATAAGCAGT | NC_045512.2 | 3′→5′ | 15499 | 15515 |
| 2217 | ACTATTAGCATAAGCAG | NC_045512.2 | 3′→5′ | 15500 | 15516 |
| 2218 | CACTATTAGCATAAGCA | NC_045512.2 | 3′→5′ | 15501 | 15517 |
| 2219 | ACACTATTAGCATAAGC | NC_045512.2 | 3′→5′ | 15502 | 15518 |
| 2220 | GCTTGACAAATGTTAAA | NC_045512.2 | 3′→5′ | 15520 | 15536 |
| 2221 | CTATAGAGACACTCATA | NC_045512.2 | 3′→5′ | 15622 | 15638 |
| 2222 | TCTATAGAGACACTCAT | NC_045512.2 | 3′→5′ | 15623 | 15639 |
| 2223 | TTCTATAGAGACACTCA | NC_045512.2 | 3′→5′ | 15624 | 15640 |
| 2224 | TTTCTATAGAGACACTC | NC_045512.2 | 3′→5′ | 15625 | 15641 |
| 2225 | ATTTCTATAGAGACACT | NC_045512.2 | 3′→5′ | 15626 | 15642 |
| 2226 | TATTTCTATAGAGACAC | NC_045512.2 | 3′→5′ | 15627 | 15643 |
| 2227 | CTATTTCTATAGAGACA | NC_045512.2 | 3′→5′ | 15628 | 15644 |
| 2228 | AGGTCAGTCTCAGTCCA | NC_045512.2 | 3′→5′ | 15838 | 15854 |
| 2229 | AAGGTCAGTCTCAGTCC | NC_045512.2 | 3′→5′ | 15839 | 15855 |
| 2230 | TAAGGTCAGTCTCAGTC | NC_045512.2 | 3′→5′ | 15840 | 15856 |
| 2231 | GTAAGGTCAGTCTCAGT | NC_045512.2 | 3′→5′ | 15841 | 15857 |
| 2232 | AGTAAGGTCAGTCTCAG | NC_045512.2 | 3′→5′ | 15842 | 15858 |
| 2233 | TAGTAAGGTCAGTCTCA | NC_045512.2 | 3′→5′ | 15843 | 15859 |
| 2234 | TTAGTAAGGTCAGTCTC | NC_045512.2 | 3′→5′ | 15844 | 15860 |
| 2235 | TTTAGTAAGGTCAGTCT | NC_045512.2 | 3′→5′ | 15845 | 15861 |
| 2236 | CTTTAGTAAGGTCAGTC | NC_045512.2 | 3′→5′ | 15846 | 15862 |
| 2237 | CCTTTAGTAAGGTCAGT | NC_045512.2 | 3′→5′ | 15847 | 15863 |
| 2238 | TCCTTTAGTAAGGTCAG | NC_045512.2 | 3′→5′ | 15848 | 15864 |
| 2239 | GTCCTTTAGTAAGGTCA | NC_045512.2 | 3′→5′ | 15849 | 15865 |
| 2240 | GGTCCTTTAGTAAGGTC | NC_045512.2 | 3′→5′ | 15850 | 15866 |
| 2241 | AGGTCCTTTAGTAAGGT | NC_045512.2 | 3′→5′ | 15851 | 15867 |
| 2242 | GAGGTCCTTTAGTAAGG | NC_045512.2 | 3′→5′ | 15852 | 15868 |
| 2243 | TGAGGTCCTTTAGTAAG | NC_045512.2 | 3′→5′ | 15853 | 15869 |
| 2244 | TTAACTAGCATTGTATG | NC_045512.2 | 3′→5′ | 15886 | 15902 |
| 2245 | TTTAACTAGCATTGTAT | NC_045512.2 | 3′→5′ | 15887 | 15903 |
| 2246 | GTTTAACTAGCATTGTA | NC_045512.2 | 3′→5′ | 15888 | 15904 |
| 2247 | TGTTTAACTAGCATTGT | NC_045512.2 | 3′→5′ | 15889 | 15905 |
| 2248 | GATGGATCTGGGTAAGG | NC_045512.2 | 3′→5′ | 15928 | 15944 |
| 2249 | AGTGTACCATCTGTTTT | NC_045512.2 | 3′→5′ | 15985 | 16001 |
| 2250 | AAGTGTACCATCTGTTT | NC_045512.2 | 3′→5′ | 15986 | 16002 |
| 2251 | TAAGTGTACCATCTGTT | NC_045512.2 | 3′→5′ | 15987 | 16003 |
| 2252 | ATAAGTGTACCATCTGT | NC_045512.2 | 3′→5′ | 15988 | 16004 |
| 2253 | CATAAGTGTACCATCTG | NC_045512.2 | 3′→5′ | 15989 | 16005 |
| 2254 | TCATAAGTGTACCATCT | NC_045512.2 | 3′→5′ | 15990 | 16006 |
| 2255 | ATCATAAGTGTACCATC | NC_045512.2 | 3′→5′ | 15991 | 16007 |
| 2256 | AATCATAAGTGTACCAT | NC_045512.2 | 3′→5′ | 15992 | 16008 |
| 2257 | CAATCATAAGTGTACCA | NC_045512.2 | 3′→5′ | 15993 | 16009 |
| 2258 | TCAATCATAAGTGTACC | NC_045512.2 | 3′→5′ | 15994 | 16010 |
| 2259 | GCATACTCCTGATTAGG | NC_045512.2 | 3′→5′ | 16057 | 16073 |
| 2260 | AGCATACTCCTGATTAG | NC_045512.2 | 3′→5′ | 16058 | 16074 |
| 2261 | CAGCATACTCCTGATTA | NC_045512.2 | 3′→5′ | 16059 | 16075 |
| 2262 | TCAGCATACTCCTGATT | NC_045512.2 | 3′→5′ | 16060 | 16076 |
| 2263 | ATCAGCATACTCCTGAT | NC_045512.2 | 3′→5′ | 16061 | 16077 |
| 2264 | CATCAGCATACTCCTGA | NC_045512.2 | 3′→5′ | 16062 | 16078 |
| 2265 | ACATCAGCATACTCCTG | NC_045512.2 | 3′→5′ | 16063 | 16079 |
| 2266 | TAAAACTCAGGTTCCCA | NC_045512.2 | 3′→5′ | 16186 | 16202 |
| 2267 | ATAAAACTCAGGTTCCC | NC_045512.2 | 3′→5′ | 16187 | 16203 |
| 2268 | CATAAAACTCAGGTTCC | NC_045512.2 | 3′→5′ | 16188 | 16204 |
| 2269 | TCATAAAACTCAGGTTC | NC_045512.2 | 3′→5′ | 16189 | 16205 |
| 2270 | ATAGCTCATACCTCCTA | NC_045512.2 | 3′→5′ | 16430 | 16446 |
| 2271 | AATAGCTCATACCTCCT | NC_045512.2 | 3′→5′ | 16431 | 16447 |
| 2272 | TAATAGCTCATACCTCC | NC_045512.2 | 3′→5′ | 16432 | 16448 |
| 2273 | AATGGAAAACTAATGGG | NC_045512.2 | 3′→5′ | 16468 | 16484 |
| 2274 | TAACCAGTAAAGACATA | NC_045512.2 | 3′→5′ | 16774 | 16790 |
| 2275 | TCAAAGGTGTACTCTCC | NC_045512.2 | 3′→5′ | 16822 | 16838 |
| 2276 | TTCAAAGGTGTACTCTC | NC_045512.2 | 3′→5′ | 16823 | 16839 |
| 2277 | TTTCAAAGGTGTACTCT | NC_045512.2 | 3′→5′ | 16824 | 16840 |
| 2278 | TTTTCAAAGGTGTACTC | NC_045512.2 | 3′→5′ | 16825 | 16841 |
| 2279 | TTTTTCAAAGGTGTACT | NC_045512.2 | 3′→5′ | 16826 | 16842 |
| 2280 | CTTTTTCAAAGGTGTAC | NC_045512.2 | 3′→5′ | 16827 | 16843 |
| 2281 | CCTTTTTCAAAGGTGTA | NC_045512.2 | 3′→5′ | 16828 | 16844 |
| 2282 | ACCTTTTTCAAAGGTGT | NC_045512.2 | 3′→5′ | 16829 | 16845 |
| 2283 | CACCTTTTTCAAAGGTG | NC_045512.2 | 3′→5′ | 16830 | 16846 |
| 2284 | TCACCTTTTTCAAAGGT | NC_045512.2 | 3′→5′ | 16831 | 16847 |
| 2285 | GTCACCTTTTTCAAAGG | NC_045512.2 | 3′→5′ | 16832 | 16848 |
| 2286 | AGTCACCTTTTTCAAAG | NC_045512.2 | 3′→5′ | 16833 | 16849 |
| 2287 | TAGTCACCTTTTTCAAA | NC_045512.2 | 3′→5′ | 16834 | 16850 |
| 2288 | ATAGTCACCTTTTTCAA | NC_045512.2 | 3′→5′ | 16835 | 16851 |
| 2289 | CATAGTCACCTTTTTCA | NC_045512.2 | 3′→5′ | 16836 | 16852 |
| 2290 | CCATAGTCACCTTTTTC | NC_045512.2 | 3′→5′ | 16837 | 16853 |
| 2291 | ACCATAGTCACCTTTTT | NC_045512.2 | 3′→5′ | 16838 | 16854 |
| 2292 | CACCATAGTCACCTTTT | NC_045512.2 | 3′→5′ | 16839 | 16855 |
| 2293 | TCACCATAGTCACCTTT | NC_045512.2 | 3′→5′ | 16840 | 16856 |
| 2294 | ATCACCATAGTCACCTT | NC_045512.2 | 3′→5′ | 16841 | 16857 |
| 2295 | CATCACCATAGTCACCT | NC_045512.2 | 3′→5′ | 16842 | 16858 |
| 2296 | GCATCACCATAGTCACC | NC_045512.2 | 3′→5′ | 16843 | 16859 |
| 2297 | AGCATCACCATAGTCAC | NC_045512.2 | 3′→5′ | 16844 | 16860 |
| 2298 | CAGCATCACCATAGTCA | NC_045512.2 | 3′→5′ | 16845 | 16861 |
| 2299 | ACAGCATCACCATAGTC | NC_045512.2 | 3′→5′ | 16846 | 16862 |
| 2300 | AACAGCATCACCATAGT | NC_045512.2 | 3′→5′ | 16847 | 16863 |
| 2301 | CAACAGCATCACCATAG | NC_045512.2 | 3′→5′ | 16848 | 16864 |
| 2302 | ACAACAGCATCACCATA | NC_045512.2 | 3′→5′ | 16849 | 16865 |
| 2303 | TGCTCTTGTGGCACTAG | NC_045512.2 | 3′→5′ | 16954 | 16970 |
| 2304 | GTGCTCTTGTGGCACTA | NC_045512.2 | 3′→5′ | 16955 | 16971 |
| 2305 | AGTGCTCTTGTGGCACT | NC_045512.2 | 3′→5′ | 16956 | 16972 |
| 2306 | TAGTGCTCTTGTGGCAC | NC_045512.2 | 3′→5′ | 16957 | 16973 |
| 2307 | ATAGTGCTCTTGTGGCA | NC_045512.2 | 3′→5′ | 16958 | 16974 |
| 2308 | CATAGTGCTCTTGTGGC | NC_045512.2 | 3′→5′ | 16959 | 16975 |
| 2309 | ACATAGTGCTCTTGTGG | NC_045512.2 | 3′→5′ | 16960 | 16976 |
| 2310 | GAAAACTCATCTGAGAT | NC_045512.2 | 3′→5′ | 17008 | 17024 |
| 2311 | AGAAAACTCATCTGAGA | NC_045512.2 | 3′→5′ | 17009 | 17025 |
| 2312 | TAGAAAACTCATCTGAG | NC_045512.2 | 3′→5′ | 17010 | 17026 |
| 2313 | CTAGAAAACTCATCTGA | NC_045512.2 | 3′→5′ | 17011 | 17027 |
| 2314 | GCTAGAAAACTCATCTG | NC_045512.2 | 3′→5′ | 17012 | 17028 |
| 2315 | TGCTAGAAAACTCATCT | NC_045512.2 | 3′→5′ | 17013 | 17029 |
| 2316 | TTGCTAGAAAACTCATC | NC_045512.2 | 3′→5′ | 17014 | 17030 |
| 2317 | ATTGCTAGAAAACTCAT | NC_045512.2 | 3′→5′ | 17015 | 17031 |
| 2318 | CATTGCTAGAAAACTCA | NC_045512.2 | 3′→5′ | 17016 | 17032 |
| 2319 | ACATTGCTAGAAAACTC | NC_045512.2 | 3′→5′ | 17017 | 17033 |
| 2320 | AACATTGCTAGAAAACT | NC_045512.2 | 3′→5′ | 17018 | 17034 |
| 2321 | CAACATTGCTAGAAAAC | NC_045512.2 | 3′→5′ | 17019 | 17035 |
| 2322 | GCAACATTGCTAGAAAA | NC_045512.2 | 3′→5′ | 17020 | 17036 |
| 2323 | TGCAACATTGCTAGAAA | NC_045512.2 | 3′→5′ | 17021 | 17037 |
| 2324 | TTGCAACATTGCTAGAA | NC_045512.2 | 3′→5′ | 17022 | 17038 |
| 2325 | TTTGCAACATTGCTAGA | NC_045512.2 | 3′→5′ | 17023 | 17039 |
| 2326 | ATTTGCAACATTGCTAG | NC_045512.2 | 3′→5′ | 17024 | 17040 |
| 2327 | AATTTGCAACATTGCTA | NC_045512.2 | 3′→5′ | 17025 | 17041 |
| 2328 | TAATTTGCAACATTGCT | NC_045512.2 | 3′→5′ | 17026 | 17042 |
| 2329 | CCAGTACCAGGTGGTCC | NC_045512.2 | 3′→5′ | 17080 | 17096 |
| 2330 | ACCAGTACCAGGTGGTC | NC_045512.2 | 3′→5′ | 17081 | 17097 |
| 2331 | TACCAGTACCAGGTGGT | NC_045512.2 | 3′→5′ | 17082 | 17098 |
| 2332 | TTACCAGTACCAGGTGG | NC_045512.2 | 3′→5′ | 17083 | 17099 |
| 2333 | CTTACCAGTACCAGGTG | NC_045512.2 | 3′→5′ | 17084 | 17100 |
| 2334 | TCTTACCAGTACCAGGT | NC_045512.2 | 3′→5′ | 17085 | 17101 |
| 2335 | CTCTTACCAGTACCAGG | NC_045512.2 | 3′→5′ | 17086 | 17102 |
| 2336 | ACTCTTACCAGTACCAG | NC_045512.2 | 3′→5′ | 17087 | 17103 |
| 2337 | GACTCTTACCAGTACCA | NC_045512.2 | 3′→5′ | 17088 | 17104 |
| 2338 | TGACTCTTACCAGTACC | NC_045512.2 | 3′→5′ | 17089 | 17105 |
| 2339 | ATGACTCTTACCAGTAC | NC_045512.2 | 3′→5′ | 17090 | 17106 |
| 2340 | AATGACTCTTACCAGTA | NC_045512.2 | 3′→5′ | 17091 | 17107 |
| 2341 | AAATGACTCTTACCAGT | NC_045512.2 | 3′→5′ | 17092 | 17108 |
| 2342 | AAAATGACTCTTACCAG | NC_045512.2 | 3′→5′ | 17093 | 17109 |
| 2343 | CAAAATGACTCTTACCA | NC_045512.2 | 3′→5′ | 17094 | 17110 |
| 2344 | GCAAAATGACTCTTACC | NC_045512.2 | 3′→5′ | 17095 | 17111 |
| 2345 | TACACTATGCGAGCAGA | NC_045512.2 | 3′→5′ | 17137 | 17153 |
| 2346 | ATACACTATGCGAGCAG | NC_045512.2 | 3′→5′ | 17138 | 17154 |
| 2347 | TATACACTATGCGAGCA | NC_045512.2 | 3′→5′ | 17139 | 17155 |
| 2348 | GTATACACTATGCGAGC | NC_045512.2 | 3′→5′ | 17140 | 17156 |
| 2349 | GAATTCACTTTGAATTT | NC_045512.2 | 3′→5′ | 17269 | 17285 |
| 2350 | TGAATTCACTTTGAATT | NC_045512.2 | 3′→5′ | 17270 | 17286 |
| 2351 | TTGAATTCACTTTGAAT | NC_045512.2 | 3′→5′ | 17271 | 17287 |
| 2352 | GTTGAATTCACTTTGAA | NC_045512.2 | 3′→5′ | 17272 | 17288 |
| 2353 | TGTTGAATTCACTTTGA | NC_045512.2 | 3′→5′ | 17273 | 17289 |
| 2354 | GCATTGACAACACTCAA | NC_045512.2 | 3′→5′ | 17386 | 17402 |
| 2355 | AACATGTCTGGACCTAT | NC_045512.2 | 3′→5′ | 17530 | 17546 |
| 2356 | GAACATGTCTGGACCTA | NC_045512.2 | 3′→5′ | 17531 | 17547 |
| 2357 | GGAACATGTCTGGACCT | NC_045512.2 | 3′→5′ | 17532 | 17548 |
| 2358 | AGGAACATGTCTGGACC | NC_045512.2 | 3′→5′ | 17533 | 17549 |
| 2359 | ATTTCAGCAGGACAACG | NC_045512.2 | 3′→5′ | 17563 | 17579 |
| 2360 | AATTTCAGCAGGACAAC | NC_045512.2 | 3′→5′ | 17564 | 17580 |
| 2361 | CAATTTCAGCAGGACAA | NC_045512.2 | 3′→5′ | 17565 | 17581 |
| 2362 | ACAATTTCAGCAGGACA | NC_045512.2 | 3′→5′ | 17566 | 17582 |
| 2363 | GCAGATGAAACATCATG | NC_045512.2 | 3′→5′ | 17680 | 17696 |
| 2364 | TGCAGATGAAACATCAT | NC_045512.2 | 3′→5′ | 17681 | 17697 |
| 2365 | TTGCAGATGAAACATCA | NC_045512.2 | 3′→5′ | 17682 | 17698 |
| 2366 | ATTGCAGATGAAACATC | NC_045512.2 | 3′→5′ | 17683 | 17699 |
| 2367 | GCTTTTCTCCAAGCAGG | NC_045512.2 | 3′→5′ | 17746 | 17762 |
| 2368 | AGCTTTTCTCCAAGCAG | NC_045512.2 | 3′→5′ | 17747 | 17763 |
| 2369 | CAGCTTTTCTCCAAGCA | NC_045512.2 | 3′→5′ | 17748 | 17764 |
| 2370 | ACAGCTTTTCTCCAAGC | NC_045512.2 | 3′→5′ | 17749 | 17765 |
| 2371 | AATATGACATAGTCATA | NC_045512.2 | 3′→5′ | 17857 | 17873 |
| 2372 | GAATATGACATAGTCAT | NC_045512.2 | 3′→5′ | 17858 | 17874 |
| 2373 | TGAATATGACATAGTCA | NC_045512.2 | 3′→5′ | 17859 | 17875 |
| 2374 | GTGAATATGACATAGTC | NC_045512.2 | 3′→5′ | 17860 | 17876 |
| 2375 | CTATCAGACATTATGCA | NC_045512.2 | 3′→5′ | 17956 | 17972 |
| 2376 | TCTATCAGACATTATGC | NC_045512.2 | 3′→5′ | 17957 | 17973 |
| 2377 | CTCTATCAGACATTATG | NC_045512.2 | 3′→5′ | 17958 | 17974 |
| 2378 | TCTCTATCAGACATTAT | NC_045512.2 | 3′→5′ | 17959 | 17975 |
| 2379 | TGTGTAGGTGCCTGTGT | NC_045512.2 | 3′→5′ | 18100 | 18116 |
| 2380 | GTGTGTAGGTGCCTGTG | NC_045512.2 | 3′→5′ | 18101 | 18117 |
| 2381 | GGTGTGTAGGTGCCTGT | NC_045512.2 | 3′→5′ | 18102 | 18118 |
| 2382 | AGGTGTGTAGGTGCCTG | NC_045512.2 | 3′→5′ | 18103 | 18119 |
| 2383 | GAGGTGTGTAGGTGCCT | NC_045512.2 | 3′→5′ | 18104 | 18120 |
| 2384 | TGAGGTGTGTAGGTGCC | NC_045512.2 | 3′→5′ | 18105 | 18121 |
| 2385 | CTGAGGTGTGTAGGTGC | NC_045512.2 | 3′→5′ | 18106 | 18122 |
| 2386 | ATCATAGAGATGAGTCT | NC_045512.2 | 3′→5′ | 18196 | 18212 |
| 2387 | CATCATAGAGATGAGTC | NC_045512.2 | 3′→5′ | 18197 | 18213 |
| 2388 | CCATCATAGAGATGAGT | NC_045512.2 | 3′→5′ | 18198 | 18214 |
| 2389 | CCCATCATAGAGATGAG | NC_045512.2 | 3′→5′ | 18199 | 18215 |
| 2390 | ACCCATCATAGAGATGA | NC_045512.2 | 3′→5′ | 18200 | 18216 |
| 2391 | AACCCATCATAGAGATG | NC_045512.2 | 3′→5′ | 18201 | 18217 |
| 2392 | AAACCCATCATAGAGAT | NC_045512.2 | 3′→5′ | 18202 | 18218 |
| 2393 | CGCTTAACAAAGCACTC | NC_045512.2 | 3′→5′ | 18889 | 18905 |
| 2394 | ACATTTTCTAAACTCTG | NC_045512.2 | 3′→5′ | 19618 | 19634 |
| 2395 | CACATTTTCTAAACTCT | NC_045512.2 | 3′→5′ | 19619 | 19635 |
| 2396 | CCACATTTTCTAAACTC | NC_045512.2 | 3′→5′ | 19620 | 19636 |
| 2397 | GCCACATTTTCTAAACT | NC_045512.2 | 3′→5′ | 19621 | 19637 |
| 2398 | AGCCACATTTTCTAAAC | NC_045512.2 | 3′→5′ | 19622 | 19638 |
| 2399 | AAGCCACATTTTCTAAA | NC_045512.2 | 3′→5′ | 19623 | 19639 |
| 2400 | TGTCCATCAAAGTGTCC | NC_045512.2 | 3′→5′ | 19657 | 19673 |
| 2401 | TTAGCCCAAAGCTCAAA | NC_045512.2 | 3′→5′ | 19783 | 19799 |
| 2402 | CTTAGCCCAAAGCTCAA | NC_045512.2 | 3′→5′ | 19784 | 19800 |
| 2403 | GCTTAGCCCAAAGCTCA | NC_045512.2 | 3′→5′ | 19785 | 19801 |
| 2404 | CGCTTAGCCCAAAGCTC | NC_045512.2 | 3′→5′ | 19786 | 19802 |
| 2405 | CCCAAATTATTGAGTAT | NC_045512.2 | 3′→5′ | 19831 | 19847 |
| 2406 | ACCCAAATTATTGAGTA | NC_045512.2 | 3′→5′ | 19832 | 19848 |
| 2407 | CACCCAAATTATTGAGT | NC_045512.2 | 3′→5′ | 19833 | 19849 |
| 2408 | ACACCCAAATTATTGAG | NC_045512.2 | 3′→5′ | 19834 | 19850 |
| 2409 | ATTAATGTGACTCCATT | NC_045512.2 | 3′→5′ | 20107 | 20123 |
| 2410 | AATTAATGTGACTCCAT | NC_045512.2 | 3′→5′ | 20108 | 20124 |
| 2411 | CAATTAATGTGACTCCA | NC_045512.2 | 3′→5′ | 20109 | 20125 |
| 2412 | CCAATTAATGTGACTCC | NC_045512.2 | 3′→5′ | 20110 | 20126 |
| 2413 | TCCAATTAATGTGACTC | NC_045512.2 | 3′→5′ | 20111 | 20127 |
| 2414 | CTCCAATTAATGTGACT | NC_045512.2 | 3′→5′ | 20112 | 20128 |
| 2415 | TCTCCAATTAATGTGAC | NC_045512.2 | 3′→5′ | 20113 | 20129 |
| 2416 | TTCTCCAATTAATGTGA | NC_045512.2 | 3′→5′ | 20114 | 20130 |
| 2417 | GCGACATTCATCATTAT | NC_045512.2 | 3′→5′ | 20776 | 20792 |
| 2418 | TGCGACATTCATCATTA | NC_045512.2 | 3′→5′ | 20777 | 20793 |
| 2419 | TTGCGACATTCATCATT | NC_045512.2 | 3′→5′ | 20778 | 20794 |
| 2420 | TTTGCGACATTCATCAT | NC_045512.2 | 3′→5′ | 20779 | 20795 |
| 2421 | TCAGCATTCCAAGAATG | NC_045512.2 | 3′→5′ | 21178 | 21194 |
| 2422 | CCAATTAAAAATGCTTC | NC_045512.2 | 3′→5′ | 21265 | 21281 |
| 2423 | CTGTTGTTTTCTCTAAT | NC_045512.2 | 3′→5′ | 21502 | 21518 |
| 2424 | TCTGTTGTTTTCTCTAA | NC_045512.2 | 3′→5′ | 21503 | 21519 |
| 2425 | CTCTGTTGTTTTCTCTA | NC_045512.2 | 3′→5′ | 21504 | 21520 |
| 2426 | ACTCTGTTGTTTTCTCT | NC_045512.2 | 3′→5′ | 21505 | 21521 |
| 2427 | AACTCTGTTGTTTTCTC | NC_045512.2 | 3′→5′ | 21506 | 21522 |
| 2428 | CAACTCTGTTGTTTTCT | NC_045512.2 | 3′→5′ | 21507 | 21523 |
| 2429 | ACAACTCTGTTGTTTTC | NC_045512.2 | 3′→5′ | 21508 | 21524 |
| 2430 | AATATTTGTGAAAAATT | NC_045512.2 | 3′→5′ | 23963 | 23979 |
| 2431 | GCCATTTGCATAGCAAA | NC_045512.2 | 3′→5′ | 24254 | 24270 |
| 2432 | TTCTCATAGAGAACATT | NC_045512.2 | 3′→5′ | 24302 | 24318 |
| 2433 | GTTCTCATAGAGAACAT | NC_045512.2 | 3′→5′ | 24303 | 24319 |
| 2434 | GGTTCTCATAGAGAACA | NC_045512.2 | 3′→5′ | 24304 | 24320 |
| 2435 | TGGTTCTCATAGAGAAC | NC_045512.2 | 3′→5′ | 24305 | 24321 |
| 2436 | TTGGTTCTCATAGAGAA | NC_045512.2 | 3′→5′ | 24306 | 24322 |
| 2437 | TTTGGTTCTCATAGAGA | NC_045512.2 | 3′→5′ | 24307 | 24323 |
| 2438 | TTTTGGTTCTCATAGAG | NC_045512.2 | 3′→5′ | 24308 | 24324 |
| 2439 | TTTTTGGTTCTCATAGA | NC_045512.2 | 3′→5′ | 24309 | 24325 |
| 2440 | CTAAGTTGTTTAACAAG | NC_045512.2 | 3′→5′ | 24446 | 24462 |
| 2441 | GCTAAGTTGTTTAACAA | NC_045512.2 | 3′→5′ | 24447 | 24463 |
| 2442 | AGCTAAGTTGTTTAACA | NC_045512.2 | 3′→5′ | 24448 | 24464 |
| 2443 | GAGCTAAGTTGTTTAAC | NC_045512.2 | 3′→5′ | 24449 | 24465 |
| 2444 | GCAAGATTAGCAGAAGC | NC_045512.2 | 3′→5′ | 24620 | 24636 |
| 2445 | AGCAAGATTAGCAGAAG | NC_045512.2 | 3′→5′ | 24621 | 24637 |
| 2446 | CAGCAAGATTAGCAGAA | NC_045512.2 | 3′→5′ | 24622 | 24638 |
| 2447 | GCAGCAAGATTAGCAGA | NC_045512.2 | 3′→5′ | 24623 | 24639 |
| 2448 | AGCAGCAAGATTAGCAG | NC_045512.2 | 3′→5′ | 24624 | 24640 |
| 2449 | TAGCAGCAAGATTAGCA | NC_045512.2 | 3′→5′ | 24625 | 24641 |
| 2450 | GTAGCAGCAAGATTAGC | NC_045512.2 | 3′→5′ | 24626 | 24642 |
| 2451 | AGTAGCAGCAAGATTAG | NC_045512.2 | 3′→5′ | 24627 | 24643 |
| 2452 | TAGTAGCAGCAAGATTA | NC_045512.2 | 3′→5′ | 24628 | 24644 |
| 2453 | TTAGTAGCAGCAAGATT | NC_045512.2 | 3′→5′ | 24629 | 24645 |
| 2454 | TTTAGTAGCAGCAAGAT | NC_045512.2 | 3′→5′ | 24630 | 24646 |
| 2455 | TTTTAGTAGCAGCAAGA | NC_045512.2 | 3′→5′ | 24631 | 24647 |
| 2456 | ATTTTAGTAGCAGCAAG | NC_045512.2 | 3′→5′ | 24632 | 24648 |
| 2457 | CATTTTAGTAGCAGCAA | NC_045512.2 | 3′→5′ | 24633 | 24649 |
| 2458 | ACATTTTAGTAGCAGCA | NC_045512.2 | 3′→5′ | 24634 | 24650 |
| 2459 | GACATTTTAGTAGCAGC | NC_045512.2 | 3′→5′ | 24635 | 24651 |
| 2460 | CTTTTTGATTGTCCAAG | NC_045512.2 | 3′→5′ | 24662 | 24678 |
| 2461 | TCTTTTTGATTGTCCAA | NC_045512.2 | 3′→5′ | 24663 | 24679 |
| 2462 | CTCTTTTTGATTGTCCA | NC_045512.2 | 3′→5′ | 24664 | 24680 |
| 2463 | ACTCTTTTTGATTGTCC | NC_045512.2 | 3′→5′ | 24665 | 24681 |
| 2464 | AACTCTTTTTGATTGTC | NC_045512.2 | 3′→5′ | 24666 | 24682 |
| 2465 | CAACTCTTTTTGATTGT | NC_045512.2 | 3′→5′ | 24667 | 24683 |
| 2466 | TCAACTCTTTTTGATTG | NC_045512.2 | 3′→5′ | 24668 | 24684 |
| 2467 | TCTGGTGATGTATGATT | NC_045512.2 | 3′→5′ | 25034 | 25050 |
| 2468 | ATCTGGTGATGTATGAT | NC_045512.2 | 3′→5′ | 25035 | 25051 |
| 2469 | CATCTGGTGATGTATGA | NC_045512.2 | 3′→5′ | 25036 | 25052 |
| 2470 | ACATCTGGTGATGTATG | NC_045512.2 | 3′→5′ | 25037 | 25053 |
| 2471 | AACATCTGGTGATGTAT | NC_045512.2 | 3′→5′ | 25038 | 25054 |
| 2472 | CAACATCTGGTGATGTA | NC_045512.2 | 3′→5′ | 25039 | 25055 |
| 2473 | TCAACATCTGGTGATGT | NC_045512.2 | 3′→5′ | 25040 | 25056 |
| 2474 | ATCAACATCTGGTGATG | NC_045512.2 | 3′→5′ | 25041 | 25057 |
| 2475 | GAGGCGGTCAATTTCTT | NC_045512.2 | 3′→5′ | 25104 | 25120 |
| 2476 | TGAGGCGGTCAATTTCT | NC_045512.2 | 3′→5′ | 25105 | 25121 |
| 2477 | TTGAGGCGGTCAATTTC | NC_045512.2 | 3′→5′ | 25106 | 25122 |
| 2478 | ATTGAGGCGGTCAATTT | NC_045512.2 | 3′→5′ | 25107 | 25123 |
| 2479 | CATTGAGGCGGTCAATT | NC_045512.2 | 3′→5′ | 25108 | 25124 |
| 2480 | TCATTGAGGCGGTCAAT | NC_045512.2 | 3′→5′ | 25109 | 25125 |
| 2481 | CTCATTGAGGCGGTCAA | NC_045512.2 | 3′→5′ | 25110 | 25126 |
| 2482 | CCTCATTGAGGCGGTCA | NC_045512.2 | 3′→5′ | 25111 | 25127 |
| 2483 | ACCTCATTGAGGCGGTC | NC_045512.2 | 3′→5′ | 25112 | 25128 |
| 2484 | GTGTAATGTAATTTGAC | NC_045512.2 | 3′→5′ | 25364 | 25380 |
| 2485 | TGTGTAATGTAATTTGA | NC_045512.2 | 3′→5′ | 25365 | 25381 |
| 2486 | ATGTGTAATGTAATTTG | NC_045512.2 | 3′→5′ | 25366 | 25382 |
| 2487 | TATGTGTAATGTAATTT | NC_045512.2 | 3′→5′ | 25367 | 25383 |
| 2488 | TTATGTGTAATGTAATT | NC_045512.2 | 3′→5′ | 25368 | 25384 |
| 2489 | TTTATGTGTAATGTAAT | NC_045512.2 | 3′→5′ | 25369 | 25385 |
| 2490 | GTTTATGTGTAATGTAA | NC_045512.2 | 3′→5′ | 25370 | 25386 |
| 2491 | CGTTTATGTGTAATGTA | NC_045512.2 | 3′→5′ | 25371 | 25387 |
| 2492 | AGGGAGTGAGGCTTGTA | NC_045512.2 | 3′→5′ | 25502 | 25518 |
| 2493 | AAGGGAGTGAGGCTTGT | NC_045512.2 | 3′→5′ | 25503 | 25519 |
| 2494 | AAAGGGAGTGAGGCTTG | NC_045512.2 | 3′→5′ | 25504 | 25520 |
| 2495 | GAAAGGGAGTGAGGCTT | NC_045512.2 | 3′→5′ | 25505 | 25521 |
| 2496 | CGAAAGGGAGTGAGGCT | NC_045512.2 | 3′→5′ | 25506 | 25522 |
| 2497 | CCGAAAGGGAGTGAGGC | NC_045512.2 | 3′→5′ | 25507 | 25523 |
| 2498 | TCCGAAAGGGAGTGAGG | NC_045512.2 | 3′→5′ | 25508 | 25524 |
| 2499 | ATCCGAAAGGGAGTGAG | NC_045512.2 | 3′→5′ | 25509 | 25525 |
| 2500 | CATCCGAAAGGGAGTGA | NC_045512.2 | 3′→5′ | 25510 | 25526 |
| 2501 | CCATCCGAAAGGGAGTG | NC_045512.2 | 3′→5′ | 25511 | 25527 |
| 2502 | GCCATCCGAAAGGGAGT | NC_045512.2 | 3′→5′ | 25512 | 25528 |
| 2503 | AGCCATCCGAAAGGGAG | NC_045512.2 | 3′→5′ | 25513 | 25529 |
| 2504 | AAGCCATCCGAAAGGGA | NC_045512.2 | 3′→5′ | 25514 | 25530 |
| 2505 | CTAGTAGTCGTCGTCGG | NC_045512.2 | 3′→5′ | 26191 | 26207 |
| 2506 | GCTAGTAGTCGTCGTCG | NC_045512.2 | 3′→5′ | 26192 | 26208 |
| 2507 | CGCTAGTAGTCGTCGTC | NC_045512.2 | 3′→5′ | 26193 | 26209 |
| 2508 | ACGCTAGTAGTCGTCGT | NC_045512.2 | 3′→5′ | 26194 | 26210 |
| 2509 | CACGCTAGTAGTCGTCG | NC_045512.2 | 3′→5′ | 26195 | 26211 |
| 2510 | GCACGCTAGTAGTCGTC | NC_045512.2 | 3′→5′ | 26196 | 26212 |
| 2511 | GGCACGCTAGTAGTCGT | NC_045512.2 | 3′→5′ | 26197 | 26213 |
| 2512 | AGGCACGCTAGTAGTCG | NC_045512.2 | 3′→5′ | 26198 | 26214 |
| 2513 | AAGGCACGCTAGTAGTC | NC_045512.2 | 3′→5′ | 26199 | 26215 |
| 2514 | AAAGGCACGCTAGTAGT | NC_045512.2 | 3′→5′ | 26200 | 26216 |
| 2515 | CAAAGGCACGCTAGTAG | NC_045512.2 | 3′→5′ | 26201 | 26217 |
| 2516 | ACAAAGGCACGCTAGTA | NC_045512.2 | 3′→5′ | 26202 | 26218 |
| 2517 | TACAAAGGCACGCTAGT | NC_045512.2 | 3′→5′ | 26203 | 26219 |
| 2518 | TTACAAAGGCACGCTAG | NC_045512.2 | 3′→5′ | 26204 | 26220 |
| 2519 | CTTACAAAGGCACGCTA | NC_045512.2 | 3′→5′ | 26205 | 26221 |
| 2520 | GCTTACAAAGGCACGCT | NC_045512.2 | 3′→5′ | 26206 | 26222 |
| 2521 | TGCTTACAAAGGCACGC | NC_045512.2 | 3′→5′ | 26207 | 26223 |
| 2522 | GTGCTTACAAAGGCACG | NC_045512.2 | 3′→5′ | 26208 | 26224 |
| 2523 | TGTGCTTACAAAGGCAC | NC_045512.2 | 3′→5′ | 26209 | 26225 |
| 2524 | TTGTGCTTACAAAGGCA | NC_045512.2 | 3′→5′ | 26210 | 26226 |
| 2525 | CTTGTGCTTACAAAGGC | NC_045512.2 | 3′→5′ | 26211 | 26227 |
| 2526 | ACATAAGTTCGTACTCA | NC_045512.2 | 3′→5′ | 26232 | 26248 |
| 2527 | TACATAAGTTCGTACTC | NC_045512.2 | 3′→5′ | 26233 | 26249 |
| 2528 | GTACATAAGTTCGTACT | NC_045512.2 | 3′→5′ | 26234 | 26250 |
| 2529 | AGTACATAAGTTCGTAC | NC_045512.2 | 3′→5′ | 26235 | 26251 |
| 2530 | GAGTACATAAGTTCGTA | NC_045512.2 | 3′→5′ | 26236 | 26252 |
| 2531 | TGAGTACATAAGTTCGT | NC_045512.2 | 3′→5′ | 26237 | 26253 |
| 2532 | ATGAGTACATAAGTTCG | NC_045512.2 | 3′→5′ | 26238 | 26254 |
| 2533 | AATGAGTACATAAGTTC | NC_045512.2 | 3′→5′ | 26239 | 26255 |
| 2534 | GAATGAGTACATAAGTT | NC_045512.2 | 3′→5′ | 26240 | 26256 |
| 2535 | CGAATGAGTACATAAGT | NC_045512.2 | 3′→5′ | 26241 | 26257 |
| 2536 | ACGAATGAGTACATAAG | NC_045512.2 | 3′→5′ | 26242 | 26258 |
| 2537 | AACGAATGAGTACATAA | NC_045512.2 | 3′→5′ | 26243 | 26259 |
| 2538 | AAACGAATGAGTACATA | NC_045512.2 | 3′→5′ | 26244 | 26260 |
| 2539 | GAAACGAATGAGTACAT | NC_045512.2 | 3′→5′ | 26245 | 26261 |
| 2540 | CGAAACGAATGAGTACA | NC_045512.2 | 3′→5′ | 26246 | 26262 |
| 2541 | CCGAAACGAATGAGTAC | NC_045512.2 | 3′→5′ | 26247 | 26263 |
| 2542 | TCCGAAACGAATGAGTA | NC_045512.2 | 3′→5′ | 26248 | 26264 |
| 2543 | TTCCGAAACGAATGAGT | NC_045512.2 | 3′→5′ | 26249 | 26265 |
| 2544 | CTTCCGAAACGAATGAG | NC_045512.2 | 3′→5′ | 26250 | 26266 |
| 2545 | TCTTCCGAAACGAATGA | NC_045512.2 | 3′→5′ | 26251 | 26267 |
| 2546 | ACTATTAACGTACCTGT | NC_045512.2 | 3′→5′ | 26269 | 26285 |
| 2547 | AACTATTAACGTACCTG | NC_045512.2 | 3′→5′ | 26270 | 26286 |
| 2548 | TAACTATTAACGTACCT | NC_045512.2 | 3′→5′ | 26271 | 26287 |
| 2549 | TTAACTATTAACGTACC | NC_045512.2 | 3′→5′ | 26272 | 26288 |
| 2550 | ATTAACTATTAACGTAC | NC_045512.2 | 3′→5′ | 26273 | 26289 |
| 2551 | TATTAACTATTAACGTA | NC_045512.2 | 3′→5′ | 26274 | 26290 |
| 2552 | CTATTAACTATTAACGT | NC_045512.2 | 3′→5′ | 26275 | 26291 |
| 2553 | GCTATTAACTATTAACG | NC_045512.2 | 3′→5′ | 26276 | 26292 |
| 2554 | CGCTATTAACTATTAAC | NC_045512.2 | 3′→5′ | 26277 | 26293 |
| 2555 | ACGCTATTAACTATTAA | NC_045512.2 | 3′→5′ | 26278 | 26294 |
| 2556 | TACGCTATTAACTATTA | NC_045512.2 | 3′→5′ | 26279 | 26295 |
| 2557 | GTACGCTATTAACTATT | NC_045512.2 | 3′→5′ | 26280 | 26296 |
| 2558 | AGTACGCTATTAACTAT | NC_045512.2 | 3′→5′ | 26281 | 26297 |
| 2559 | AAGTACGCTATTAACTA | NC_045512.2 | 3′→5′ | 26282 | 26298 |
| 2560 | GAAGTACGCTATTAACT | NC_045512.2 | 3′→5′ | 26283 | 26299 |
| 2561 | AGAAGTACGCTATTAAC | NC_045512.2 | 3′→5′ | 26284 | 26300 |
| 2562 | AAGAAGTACGCTATTAA | NC_045512.2 | 3′→5′ | 26285 | 26301 |
| 2563 | AAAGAAGTACGCTATTA | NC_045512.2 | 3′→5′ | 26286 | 26302 |
| 2564 | AAAAGAAGTACGCTATT | NC_045512.2 | 3′→5′ | 26287 | 26303 |
| 2565 | AAAAAGAAGTACGCTAT | NC_045512.2 | 3′→5′ | 26288 | 26304 |
| 2566 | GAAAAAGAAGTACGCTA | NC_045512.2 | 3′→5′ | 26289 | 26305 |
| 2567 | AGAAAAAGAAGTACGCT | NC_045512.2 | 3′→5′ | 26290 | 26306 |
| 2568 | AAGAAAAAGAAGTACGC | NC_045512.2 | 3′→5′ | 26291 | 26307 |
| 2569 | CAAGAAAAAGAAGTACG | NC_045512.2 | 3′→5′ | 26292 | 26308 |
| 2570 | GCAAGAAAAAGAAGTAC | NC_045512.2 | 3′→5′ | 26293 | 26309 |
| 2571 | AGCAAGAAAAAGAAGTA | NC_045512.2 | 3′→5′ | 26294 | 26310 |
| 2572 | AAGCAAGAAAAAGAAGT | NC_045512.2 | 3′→5′ | 26295 | 26311 |
| 2573 | AAAGCAAGAAAAAGAAG | NC_045512.2 | 3′→5′ | 26296 | 26312 |
| 2574 | GAAAGCAAGAAAAAGAA | NC_045512.2 | 3′→5′ | 26297 | 26313 |
| 2575 | CGAAAGCAAGAAAAAGA | NC_045512.2 | 3′→5′ | 26298 | 26314 |
| 2576 | ACGAAAGCAAGAAAAAG | NC_045512.2 | 3′→5′ | 26299 | 26315 |
| 2577 | CACGAAAGCAAGAAAAA | NC_045512.2 | 3′→5′ | 26300 | 26316 |
| 2578 | CCACGAAAGCAAGAAAA | NC_045512.2 | 3′→5′ | 26301 | 26317 |
| 2579 | ACCACGAAAGCAAGAAA | NC_045512.2 | 3′→5′ | 26302 | 26318 |
| 2580 | TACCACGAAAGCAAGAA | NC_045512.2 | 3′→5′ | 26303 | 26319 |
| 2581 | ATACCACGAAAGCAAGA | NC_045512.2 | 3′→5′ | 26304 | 26320 |
| 2582 | AATACCACGAAAGCAAG | NC_045512.2 | 3′→5′ | 26305 | 26321 |
| 2583 | GAATACCACGAAAGCAA | NC_045512.2 | 3′→5′ | 26306 | 26322 |
| 2584 | AGAATACCACGAAAGCA | NC_045512.2 | 3′→5′ | 26307 | 26323 |
| 2585 | AAGAATACCACGAAAGC | NC_045512.2 | 3′→5′ | 26308 | 26324 |
| 2586 | CAAGAATACCACGAAAG | NC_045512.2 | 3′→5′ | 26309 | 26325 |
| 2587 | GCAAGAATACCACGAAA | NC_045512.2 | 3′→5′ | 26310 | 26326 |
| 2588 | AGCAAGAATACCACGAA | NC_045512.2 | 3′→5′ | 26311 | 26327 |
| 2589 | TAGCAAGAATACCACGA | NC_045512.2 | 3′→5′ | 26312 | 26328 |
| 2590 | CTAGCAAGAATACCACG | NC_045512.2 | 3′→5′ | 26313 | 26329 |
| 2591 | ACTAGCAAGAATACCAC | NC_045512.2 | 3′→5′ | 26314 | 26330 |
| 2592 | GTAAGGATGGCTAGTGT | NC_045512.2 | 3′→5′ | 26332 | 26348 |
| 2593 | AGTAAGGATGGCTAGTG | NC_045512.2 | 3′→5′ | 26333 | 26349 |
| 2594 | CAGTAAGGATGGCTAGT | NC_045512.2 | 3′→5′ | 26334 | 26350 |
| 2595 | GCAGTAAGGATGGCTAG | NC_045512.2 | 3′→5′ | 26335 | 26351 |
| 2596 | CGCAGTAAGGATGGCTA | NC_045512.2 | 3′→5′ | 26336 | 26352 |
| 2597 | GCGCAGTAAGGATGGCT | NC_045512.2 | 3′→5′ | 26337 | 26353 |
| 2598 | AGCGCAGTAAGGATGGC | NC_045512.2 | 3′→5′ | 26338 | 26354 |
| 2599 | AAGCGCAGTAAGGATGG | NC_045512.2 | 3′→5′ | 26339 | 26355 |
| 2600 | GAAGCGCAGTAAGGATG | NC_045512.2 | 3′→5′ | 26340 | 26356 |
| 2601 | CGAAGCGCAGTAAGGAT | NC_045512.2 | 3′→5′ | 26341 | 26357 |
| 2602 | TCGAAGCGCAGTAAGGA | NC_045512.2 | 3′→5′ | 26342 | 26358 |
| 2603 | ATCGAAGCGCAGTAAGG | NC_045512.2 | 3′→5′ | 26343 | 26359 |
| 2604 | AATCGAAGCGCAGTAAG | NC_045512.2 | 3′→5′ | 26344 | 26360 |
| 2605 | CAATCGAAGCGCAGTAA | NC_045512.2 | 3′→5′ | 26345 | 26361 |
| 2606 | ACAATCGAAGCGCAGTA | NC_045512.2 | 3′→5′ | 26346 | 26362 |
| 2607 | CACAATCGAAGCGCAGT | NC_045512.2 | 3′→5′ | 26347 | 26363 |
| 2608 | ACACAATCGAAGCGCAG | NC_045512.2 | 3′→5′ | 26348 | 26364 |
| 2609 | CACACAATCGAAGCGCA | NC_045512.2 | 3′→5′ | 26349 | 26365 |
| 2610 | GCACACAATCGAAGCGC | NC_045512.2 | 3′→5′ | 26350 | 26366 |
| 2611 | CGCACACAATCGAAGCG | NC_045512.2 | 3′→5′ | 26351 | 26367 |
| 2612 | ACGCACACAATCGAAGC | NC_045512.2 | 3′→5′ | 26352 | 26368 |
| 2613 | TACGCACACAATCGAAG | NC_045512.2 | 3′→5′ | 26353 | 26369 |
| 2614 | GTACGCACACAATCGAA | NC_045512.2 | 3′→5′ | 26354 | 26370 |
| 2615 | AGTACGCACACAATCGA | NC_045512.2 | 3′→5′ | 26355 | 26371 |
| 2616 | CAGTACGCACACAATCG | NC_045512.2 | 3′→5′ | 26356 | 26372 |
| 2617 | GCAGTACGCACACAATC | NC_045512.2 | 3′→5′ | 26357 | 26373 |
| 2618 | AGCAGTACGCACACAAT | NC_045512.2 | 3′→5′ | 26358 | 26374 |
| 2619 | CAGCAGTACGCACACAA | NC_045512.2 | 3′→5′ | 26359 | 26375 |
| 2620 | GCAGCAGTACGCACACA | NC_045512.2 | 3′→5′ | 26360 | 26376 |
| 2621 | TGCAGCAGTACGCACAC | NC_045512.2 | 3′→5′ | 26361 | 26377 |
| 2622 | TTGCAGCAGTACGCACA | NC_045512.2 | 3′→5′ | 26362 | 26378 |
| 2623 | ATTGCAGCAGTACGCAC | NC_045512.2 | 3′→5′ | 26363 | 26379 |
| 2624 | TATTGCAGCAGTACGCA | NC_045512.2 | 3′→5′ | 26364 | 26380 |
| 2625 | ATATTGCAGCAGTACGC | NC_045512.2 | 3′→5′ | 26365 | 26381 |
| 2626 | AATATTGCAGCAGTACG | NC_045512.2 | 3′→5′ | 26366 | 26382 |
| 2627 | CAATATTGCAGCAGTAC | NC_045512.2 | 3′→5′ | 26367 | 26383 |
| 2628 | ACAATATTGCAGCAGTA | NC_045512.2 | 3′→5′ | 26368 | 26384 |
| 2629 | AACAATATTGCAGCAGT | NC_045512.2 | 3′→5′ | 26369 | 26385 |
| 2630 | TAACAATATTGCAGCAG | NC_045512.2 | 3′→5′ | 26370 | 26386 |
| 2631 | TTAACAATATTGCAGCA | NC_045512.2 | 3′→5′ | 26371 | 26387 |
| 2632 | GTTAACAATATTGCAGC | NC_045512.2 | 3′→5′ | 26372 | 26388 |
| 2633 | CGTTAACAATATTGCAG | NC_045512.2 | 3′→5′ | 26373 | 26389 |
| 2634 | ACGTTAACAATATTGCA | NC_045512.2 | 3′→5′ | 26374 | 26390 |
| 2635 | CACGTTAACAATATTGC | NC_045512.2 | 3′→5′ | 26375 | 26391 |
| 2636 | TCACGTTAACAATATTG | NC_045512.2 | 3′→5′ | 26376 | 26392 |
| 2637 | CTCACGTTAACAATATT | NC_045512.2 | 3′→5′ | 26377 | 26393 |
| 2638 | ACTCACGTTAACAATAT | NC_045512.2 | 3′→5′ | 26378 | 26394 |
| 2639 | TTCAGATTTTTAACACG | NC_045512.2 | 3′→5′ | 26425 | 26441 |
| 2640 | CAGAAGATCAGGAACTC | NC_045512.2 | 3′→5′ | 26450 | 26466 |
| 2641 | CCAGAAGATCAGGAACT | NC_045512.2 | 3′→5′ | 26451 | 26467 |
| 2642 | ACCAGAAGATCAGGAAC | NC_045512.2 | 3′→5′ | 26452 | 26468 |
| 2643 | GACCAGAAGATCAGGAA | NC_045512.2 | 3′→5′ | 26453 | 26469 |
| 2644 | AGACCAGAAGATCAGGA | NC_045512.2 | 3′→5′ | 26454 | 26470 |
| 2645 | TAGACCAGAAGATCAGG | NC_045512.2 | 3′→5′ | 26455 | 26471 |
| 2646 | TTAGACCAGAAGATCAG | NC_045512.2 | 3′→5′ | 26456 | 26472 |
| 2647 | TTTAGACCAGAAGATCA | NC_045512.2 | 3′→5′ | 26457 | 26473 |
| 2648 | GTTTAGACCAGAAGATC | NC_045512.2 | 3′→5′ | 26458 | 26474 |
| 2649 | CGTTTAGACCAGAAGAT | NC_045512.2 | 3′→5′ | 26459 | 26475 |
| 2650 | TCGTTTAGACCAGAAGA | NC_045512.2 | 3′→5′ | 26460 | 26476 |
| 2651 | TTCGTTTAGACCAGAAG | NC_045512.2 | 3′→5′ | 26461 | 26477 |
| 2652 | GTTCGTTTAGACCAGAA | NC_045512.2 | 3′→5′ | 26462 | 26478 |
| 2653 | AGTTCGTTTAGACCAGA | NC_045512.2 | 3′→5′ | 26463 | 26479 |
| 2654 | TAGTTCGTTTAGACCAG | NC_045512.2 | 3′→5′ | 26464 | 26480 |
| 2655 | TTAGTTCGTTTAGACCA | NC_045512.2 | 3′→5′ | 26465 | 26481 |
| 2656 | TAAAGTTCCAAACAGAA | NC_045512.2 | 3′→5′ | 26497 | 26513 |
| 2657 | TTAAAGTTCCAAACAGA | NC_045512.2 | 3′→5′ | 26498 | 26514 |
| 2658 | ACTAGGTTCCATTGTTC | NC_045512.2 | 3′→5′ | 26574 | 26590 |
| 2659 | TACTAGGTTCCATTGTT | NC_045512.2 | 3′→5′ | 26575 | 26591 |
| 2660 | TTACTAGGTTCCATTGT | NC_045512.2 | 3′→5′ | 26576 | 26592 |
| 2661 | ATTACTAGGTTCCATTG | NC_045512.2 | 3′→5′ | 26577 | 26593 |
| 2662 | TATTACTAGGTTCCATT | NC_045512.2 | 3′→5′ | 26578 | 26594 |
| 2663 | CTATTACTAGGTTCCAT | NC_045512.2 | 3′→5′ | 26579 | 26595 |
| 2664 | CCTATTACTAGGTTCCA | NC_045512.2 | 3′→5′ | 26580 | 26596 |
| 2665 | ACCTATTACTAGGTTCC | NC_045512.2 | 3′→5′ | 26581 | 26597 |
| 2666 | AACCTATTACTAGGTTC | NC_045512.2 | 3′→5′ | 26582 | 26598 |
| 2667 | AAACCTATTACTAGGTT | NC_045512.2 | 3′→5′ | 26583 | 26599 |
| 2668 | GAAACCTATTACTAGGT | NC_045512.2 | 3′→5′ | 26584 | 26600 |
| 2669 | ACAGCAGCAAGCACAAA | NC_045512.2 | 3′→5′ | 26715 | 26731 |
| 2670 | AGCCACATCAAGCCTAC | NC_045512.2 | 3′→5′ | 26784 | 26800 |
| 2671 | AGCGTTCGTGATGTAGC | NC_045512.2 | 3′→5′ | 27033 | 27049 |
| 2672 | AAGCGTTCGTGATGTAG | NC_045512.2 | 3′→5′ | 27034 | 27050 |
| 2673 | AAAGCGTTCGTGATGTA | NC_045512.2 | 3′→5′ | 27035 | 27051 |
| 2674 | GAAAGCGTTCGTGATGT | NC_045512.2 | 3′→5′ | 27036 | 27052 |
| 2675 | AGAAAGCGTTCGTGATG | NC_045512.2 | 3′→5′ | 27037 | 27053 |
| 2676 | AAGAAAGCGTTCGTGAT | NC_045512.2 | 3′→5′ | 27038 | 27054 |
| 2677 | TAAGAAAGCGTTCGTGA | NC_045512.2 | 3′→5′ | 27039 | 27055 |
| 2678 | ATAAGAAAGCGTTCGTG | NC_045512.2 | 3′→5′ | 27040 | 27056 |
| 2679 | AATAAGAAAGCGTTCGT | NC_045512.2 | 3′→5′ | 27041 | 27057 |
| 2680 | TAATAAGAAAGCGTTCG | NC_045512.2 | 3′→5′ | 27042 | 27058 |
| 2681 | GTAATAAGAAAGCGTTC | NC_045512.2 | 3′→5′ | 27043 | 27059 |
| 2682 | TGTAATAAGAAAGCGTT | NC_045512.2 | 3′→5′ | 27044 | 27060 |
| 2683 | TTGTAATAAGAAAGCGT | NC_045512.2 | 3′→5′ | 27045 | 27061 |
| 2684 | TTTGTAATAAGAAAGCG | NC_045512.2 | 3′→5′ | 27046 | 27062 |
| 2685 | ATTTGTAATAAGAAAGC | NC_045512.2 | 3′→5′ | 27047 | 27063 |
| 2686 | AATTTGTAATAAGAAAG | NC_045512.2 | 3′→5′ | 27048 | 27064 |
| 2687 | TATGCAGCAAAACCTGA | NC_045512.2 | 3′→5′ | 27093 | 27109 |
| 2688 | GTATGCAGCAAAACCTG | NC_045512.2 | 3′→5′ | 27094 | 27110 |
| 2689 | TGTATGCAGCAAAACCT | NC_045512.2 | 3′→5′ | 27095 | 27111 |
| 2690 | AGCAAAGCAATATTGTC | NC_045512.2 | 3′→5′ | 27165 | 27181 |
| 2691 | GTTGTCACTTACTGTAC | NC_045512.2 | 3′→5′ | 27183 | 27199 |
| 2692 | TGTTGTCACTTACTGTA | NC_045512.2 | 3′→5′ | 27184 | 27200 |
| 2693 | CTGTTGTCACTTACTGT | NC_045512.2 | 3′→5′ | 27185 | 27201 |
| 2694 | TCTGTTGTCACTTACTG | NC_045512.2 | 3′→5′ | 27186 | 27202 |
| 2695 | ATCTGTTGTCACTTACT | NC_045512.2 | 3′→5′ | 27187 | 27203 |
| 2696 | CATCTGTTGTCACTTAC | NC_045512.2 | 3′→5′ | 27188 | 27204 |
| 2697 | ACATCTGTTGTCACTTA | NC_045512.2 | 3′→5′ | 27189 | 27205 |
| 2698 | AACATCTGTTGTCACTT | NC_045512.2 | 3′→5′ | 27190 | 27206 |
| 2699 | AAACATCTGTTGTCACT | NC_045512.2 | 3′→5′ | 27191 | 27207 |
| 2700 | GAAACATCTGTTGTCAC | NC_045512.2 | 3′→5′ | 27192 | 27208 |
| 2701 | TGAAACATCTGTTGTCA | NC_045512.2 | 3′→5′ | 27193 | 27209 |
| 2702 | ATGAAACATCTGTTGTC | NC_045512.2 | 3′→5′ | 27194 | 27210 |
| 2703 | GATGAAACATCTGTTGT | NC_045512.2 | 3′→5′ | 27195 | 27211 |
| 2704 | AGATGAAACATCTGTTG | NC_045512.2 | 3′→5′ | 27196 | 27212 |
| 2705 | TTCATGTTCGTTTAATC | NC_045512.2 | 3′→5′ | 27382 | 27398 |
| 2706 | TTTCATGTTCGTTTAAT | NC_045512.2 | 3′→5′ | 27383 | 27399 |
| 2707 | TTTTCATGTTCGTTTAA | NC_045512.2 | 3′→5′ | 27384 | 27400 |
| 2708 | ATTTTCATGTTCGTTTA | NC_045512.2 | 3′→5′ | 27385 | 27401 |
| 2709 | AATTTTCATGTTCGTTT | NC_045512.2 | 3′→5′ | 27386 | 27402 |
| 2710 | TAATTTTCATGTTCGTT | NC_045512.2 | 3′→5′ | 27387 | 27403 |
| 2711 | ATAATTTTCATGTTCGT | NC_045512.2 | 3′→5′ | 27388 | 27404 |
| 2712 | AATAATTTTCATGTTCG | NC_045512.2 | 3′→5′ | 27389 | 27405 |
| 2713 | GAATAATTTTCATGTTC | NC_045512.2 | 3′→5′ | 27390 | 27406 |
| 2714 | AGAATAATTTTCATGTT | NC_045512.2 | 3′→5′ | 27391 | 27407 |
| 2715 | GTACCTCTAACACACTC | NC_045512.2 | 3′→5′ | 27457 | 27473 |
| 2716 | GGTGAATTGCCCTCGTA | NC_045512.2 | 3′→5′ | 27511 | 27527 |
| 2717 | TGGTGAATTGCCCTCGT | NC_045512.2 | 3′→5′ | 27512 | 27528 |
| 2718 | ATGGTGAATTGCCCTCG | NC_045512.2 | 3′→5′ | 27513 | 27529 |
| 2719 | AATGGTGAATTGCCCTC | NC_045512.2 | 3′→5′ | 27514 | 27530 |
| 2720 | AAATGGTGAATTGCCCT | NC_045512.2 | 3′→5′ | 27515 | 27531 |
| 2721 | GAAATGGTGAATTGCCC | NC_045512.2 | 3′→5′ | 27516 | 27532 |
| 2722 | TGAAATGGTGAATTGCC | NC_045512.2 | 3′→5′ | 27517 | 27533 |
| 2723 | TCCTCTTGTCTGATGAA | NC_045512.2 | 3′→5′ | 27652 | 27668 |
| 2724 | AAATAGAAGTCAATTAA | NC_045512.2 | 3′→5′ | 27771 | 27787 |
| 2725 | CAAATAGAAGTCAATTA | NC_045512.2 | 3′→5′ | 27772 | 27788 |
| 2726 | ACAAATAGAAGTCAATT | NC_045512.2 | 3′→5′ | 27773 | 27789 |
| 2727 | CACAAATAGAAGTCAAT | NC_045512.2 | 3′→5′ | 27774 | 27790 |
| 2728 | GCACAAATAGAAGTCAA | NC_045512.2 | 3′→5′ | 27775 | 27791 |
| 2729 | AGCACAAATAGAAGTCA | NC_045512.2 | 3′→5′ | 27776 | 27792 |
| 2730 | AAGCACAAATAGAAGTC | NC_045512.2 | 3′→5′ | 27777 | 27793 |
| 2731 | AAAGCACAAATAGAAGT | NC_045512.2 | 3′→5′ | 27778 | 27794 |
| 2732 | AAAAGCACAAATAGAAG | NC_045512.2 | 3′→5′ | 27779 | 27795 |
| 2733 | AAAAAGCACAAATAGAA | NC_045512.2 | 3′→5′ | 27780 | 27796 |
| 2734 | TAAAAAGCACAAATAGA | NC_045512.2 | 3′→5′ | 27781 | 27797 |
| 2735 | CTAAAAAGCACAAATAG | NC_045512.2 | 3′→5′ | 27782 | 27798 |
| 2736 | GCTAAAAAGCACAAATA | NC_045512.2 | 3′→5′ | 27783 | 27799 |
| 2737 | GGCTAAAAAGCACAAAT | NC_045512.2 | 3′→5′ | 27784 | 27800 |
| 2738 | AGGCTAAAAAGCACAAA | NC_045512.2 | 3′→5′ | 27785 | 27801 |
| 2739 | AAGGCTAAAAAGCACAA | NC_045512.2 | 3′→5′ | 27786 | 27802 |
| 2740 | AAAGGCTAAAAAGCACA | NC_045512.2 | 3′→5′ | 27787 | 27803 |
| 2741 | GAAAGGCTAAAAAGCAC | NC_045512.2 | 3′→5′ | 27788 | 27804 |
| 2742 | AGAAAGGCTAAAAAGCA | NC_045512.2 | 3′→5′ | 27789 | 27805 |
| 2743 | CAGAAAGGCTAAAAAGC | NC_045512.2 | 3′→5′ | 27790 | 27806 |
| 2744 | GCAGAAAGGCTAAAAAG | NC_045512.2 | 3′→5′ | 27791 | 27807 |
| 2745 | AGCAGAAAGGCTAAAAA | NC_045512.2 | 3′→5′ | 27792 | 27808 |
| 2746 | TAGCAGAAAGGCTAAAA | NC_045512.2 | 3′→5′ | 27793 | 27809 |
| 2747 | ATAGCAGAAAGGCTAAA | NC_045512.2 | 3′→5′ | 27794 | 27810 |
| 2748 | AATAGCAGAAAGGCTAA | NC_045512.2 | 3′→5′ | 27795 | 27811 |
| 2749 | GAATAGCAGAAAGGCTA | NC_045512.2 | 3′→5′ | 27796 | 27812 |
| 2750 | GGAATAGCAGAAAGGCT | NC_045512.2 | 3′→5′ | 27797 | 27813 |
| 2751 | AGGAATAGCAGAAAGGC | NC_045512.2 | 3′→5′ | 27798 | 27814 |
| 2752 | AAGGAATAGCAGAAAGG | NC_045512.2 | 3′→5′ | 27799 | 27815 |
| 2753 | CAAGGAATAGCAGAAAG | NC_045512.2 | 3′→5′ | 27800 | 27816 |
| 2754 | ACAAGGAATAGCAGAAA | NC_045512.2 | 3′→5′ | 27801 | 27817 |
| 2755 | AACAAGGAATAGCAGAA | NC_045512.2 | 3′→5′ | 27802 | 27818 |
| 2756 | CATTATCAGACATTTTA | NC_045512.2 | 3′→5′ | 28270 | 28286 |
| 2757 | CCATTATCAGACATTTT | NC_045512.2 | 3′→5′ | 28271 | 28287 |
| 2758 | TCCATTATCAGACATTT | NC_045512.2 | 3′→5′ | 28272 | 28288 |
| 2759 | GTCCATTATCAGACATT | NC_045512.2 | 3′→5′ | 28273 | 28289 |
| 2760 | GGTCCATTATCAGACAT | NC_045512.2 | 3′→5′ | 28274 | 28290 |
| 2761 | GGGTCCATTATCAGACA | NC_045512.2 | 3′→5′ | 28275 | 28291 |
| 2762 | GGGGTCCATTATCAGAC | NC_045512.2 | 3′→5′ | 28276 | 28292 |
| 2763 | TGGGGTCCATTATCAGA | NC_045512.2 | 3′→5′ | 28277 | 28293 |
| 2764 | TTGGGGTCCATTATCAG | NC_045512.2 | 3′→5′ | 28278 | 28294 |
| 2765 | TTTGGGGTCCATTATCA | NC_045512.2 | 3′→5′ | 28279 | 28295 |
| 2766 | TTTTGGGGTCCATTATC | NC_045512.2 | 3′→5′ | 28280 | 28296 |
| 2767 | TTGGGTAAACCTTGGGG | NC_045512.2 | 3′→5′ | 28397 | 28413 |
| 2768 | ATTGGGTAAACCTTGGG | NC_045512.2 | 3′→5′ | 28398 | 28414 |
| 2769 | TATTGGGTAAACCTTGG | NC_045512.2 | 3′→5′ | 28399 | 28415 |
| 2770 | TTATTGGGTAAACCTTG | NC_045512.2 | 3′→5′ | 28400 | 28416 |
| 2771 | ATTATTGGGTAAACCTT | NC_045512.2 | 3′→5′ | 28401 | 28417 |
| 2772 | TATTATTGGGTAAACCT | NC_045512.2 | 3′→5′ | 28402 | 28418 |
| 2773 | GTATTATTGGGTAAACC | NC_045512.2 | 3′→5′ | 28403 | 28419 |
| 2774 | AGTATTATTGGGTAAAC | NC_045512.2 | 3′→5′ | 28404 | 28420 |
| 2775 | CAGTATTATTGGGTAAA | NC_045512.2 | 3′→5′ | 28405 | 28421 |
| 2776 | GCAGTATTATTGGGTAA | NC_045512.2 | 3′→5′ | 28406 | 28422 |
| 2777 | CGCAGTATTATTGGGTA | NC_045512.2 | 3′→5′ | 28407 | 28423 |
| 2778 | ACGCAGTATTATTGGGT | NC_045512.2 | 3′→5′ | 28408 | 28424 |
| 2779 | GACGCAGTATTATTGGG | NC_045512.2 | 3′→5′ | 28409 | 28425 |
| 2780 | AGACGCAGTATTATTGG | NC_045512.2 | 3′→5′ | 28410 | 28426 |
| 2781 | AAGACGCAGTATTATTG | NC_045512.2 | 3′→5′ | 28411 | 28427 |
| 2782 | CAAGACGCAGTATTATT | NC_045512.2 | 3′→5′ | 28412 | 28428 |
| 2783 | CCAAGACGCAGTATTAT | NC_045512.2 | 3′→5′ | 28413 | 28429 |
| 2784 | ACCAAGACGCAGTATTA | NC_045512.2 | 3′→5′ | 28414 | 28430 |
| 2785 | AACCAAGACGCAGTATT | NC_045512.2 | 3′→5′ | 28415 | 28431 |
| 2786 | GAACCAAGACGCAGTAT | NC_045512.2 | 3′→5′ | 28416 | 28432 |
| 2787 | TGAACCAAGACGCAGTA | NC_045512.2 | 3′→5′ | 28417 | 28433 |
| 2788 | GTGAACCAAGACGCAGT | NC_045512.2 | 3′→5′ | 28418 | 28434 |
| 2789 | AGCCAATTTGGTCATCT | NC_045512.2 | 3′→5′ | 28513 | 28529 |
| 2790 | TAGCCAATTTGGTCATC | NC_045512.2 | 3′→5′ | 28514 | 28530 |
| 2791 | GTAGCCAATTTGGTCAT | NC_045512.2 | 3′→5′ | 28515 | 28531 |
| 2792 | AGTAGCCAATTTGGTCA | NC_045512.2 | 3′→5′ | 28516 | 28532 |
| 2793 | TAGTAGCCAATTTGGTC | NC_045512.2 | 3′→5′ | 28517 | 28533 |
| 2794 | GTAGTAGCCAATTTGGT | NC_045512.2 | 3′→5′ | 28518 | 28534 |
| 2795 | GGTAGTAGCCAATTTGG | NC_045512.2 | 3′→5′ | 28519 | 28535 |
| 2796 | CGGTAGTAGCCAATTTG | NC_045512.2 | 3′→5′ | 28520 | 28536 |
| 2797 | TCGGTAGTAGCCAATTT | NC_045512.2 | 3′→5′ | 28521 | 28537 |
| 2798 | TTCGGTAGTAGCCAATT | NC_045512.2 | 3′→5′ | 28522 | 28538 |
| 2799 | CTTCGGTAGTAGCCAAT | NC_045512.2 | 3′→5′ | 28523 | 28539 |
| 2800 | TCTTCGGTAGTAGCCAA | NC_045512.2 | 3′→5′ | 28524 | 28540 |
| 2801 | CTCTTCGGTAGTAGCCA | NC_045512.2 | 3′→5′ | 28525 | 28541 |
| 2802 | GCTCTTCGGTAGTAGCC | NC_045512.2 | 3′→5′ | 28526 | 28542 |
| 2803 | AGCTCTTCGGTAGTAGC | NC_045512.2 | 3′→5′ | 28527 | 28543 |
| 2804 | TAGCTCTTCGGTAGTAG | NC_045512.2 | 3′→5′ | 28528 | 28544 |
| 2805 | GTAGCTCTTCGGTAGTA | NC_045512.2 | 3′→5′ | 28529 | 28545 |
| 2806 | GGTAGCTCTTCGGTAGT | NC_045512.2 | 3′→5′ | 28530 | 28546 |
| 2807 | AAGGCTCCCTCAGTTGC | NC_045512.2 | 3′→5′ | 28673 | 28689 |
| 2808 | CAAGGCTCCCTCAGTTG | NC_045512.2 | 3′→5′ | 28674 | 28690 |
| 2809 | TCAAGGCTCCCTCAGTT | NC_045512.2 | 3′→5′ | 28675 | 28691 |
| 2810 | TTCAAGGCTCCCTCAGT | NC_045512.2 | 3′→5′ | 28676 | 28692 |
| 2811 | TTGCGGGTGCCAATGTG | NC_045512.2 | 3′→5′ | 28706 | 28722 |
| 2812 | ATTGCGGGTGCCAATGT | NC_045512.2 | 3′→5′ | 28707 | 28723 |
| 2813 | GATTGCGGGTGCCAATG | NC_045512.2 | 3′→5′ | 28708 | 28724 |
| 2814 | GGATTGCGGGTGCCAAT | NC_045512.2 | 3′→5′ | 28709 | 28725 |
| 2815 | AGGATTGCGGGTGCCAA | NC_045512.2 | 3′→5′ | 28710 | 28726 |
| 2816 | GAGGAAGTTGTAGCACG | NC_045512.2 | 3′→5′ | 28744 | 28760 |
| 2817 | TGAGGAAGTTGTAGCAC | NC_045512.2 | 3′→5′ | 28745 | 28761 |
| 2818 | TTGAGGAAGTTGTAGCA | NC_045512.2 | 3′→5′ | 28746 | 28762 |
| 2819 | CTTGAGGAAGTTGTAGC | NC_045512.2 | 3′→5′ | 28747 | 28763 |
| 2820 | CCTTGAGGAAGTTGTAG | NC_045512.2 | 3′→5′ | 28748 | 28764 |
| 2821 | TCCTTGAGGAAGTTGTA | NC_045512.2 | 3′→5′ | 28749 | 28765 |
| 2822 | TTCCTTGAGGAAGTTGT | NC_045512.2 | 3′→5′ | 28750 | 28766 |
| 2823 | GTTCCTTGAGGAAGTTG | NC_045512.2 | 3′→5′ | 28751 | 28767 |
| 2824 | TGTTCCTTGAGGAAGTT | NC_045512.2 | 3′→5′ | 28752 | 28768 |
| 2825 | TTGTTCCTTGAGGAAGT | NC_045512.2 | 3′→5′ | 28753 | 28769 |
| 2826 | GTTGTTCCTTGAGGAAG | NC_045512.2 | 3′→5′ | 28754 | 28770 |
| 2827 | TGTTGTTCCTTGAGGAA | NC_045512.2 | 3′→5′ | 28755 | 28771 |
| 2828 | ATGTTGTTCCTTGAGGA | NC_045512.2 | 3′→5′ | 28756 | 28772 |
| 2829 | AATGTTGTTCCTTGAGG | NC_045512.2 | 3′→5′ | 28757 | 28773 |
| 2830 | CAATGTTGTTCCTTGAG | NC_045512.2 | 3′→5′ | 28758 | 28774 |
| 2831 | GCAATGTTGTTCCTTGA | NC_045512.2 | 3′→5′ | 28759 | 28775 |
| 2832 | GGCAATGTTGTTCCTTG | NC_045512.2 | 3′→5′ | 28760 | 28776 |
| 2833 | TGGCAATGTTGTTCCTT | NC_045512.2 | 3′→5′ | 28761 | 28777 |
| 2834 | TTGGCAATGTTGTTCCT | NC_045512.2 | 3′→5′ | 28762 | 28778 |
| 2835 | TTTGGCAATGTTGTTCC | NC_045512.2 | 3′→5′ | 28763 | 28779 |
| 2836 | TTTTGGCAATGTTGTTC | NC_045512.2 | 3′→5′ | 28764 | 28780 |
| 2837 | CTTTTGGCAATGTTGTT | NC_045512.2 | 3′→5′ | 28765 | 28781 |
| 2838 | CCTTTTGGCAATGTTGT | NC_045512.2 | 3′→5′ | 28766 | 28782 |
| 2839 | GCCTTTTGGCAATGTTG | NC_045512.2 | 3′→5′ | 28767 | 28783 |
| 2840 | AGCCTTTTGGCAATGTT | NC_045512.2 | 3′→5′ | 28768 | 28784 |
| 2841 | AAGCCTTTTGGCAATGT | NC_045512.2 | 3′→5′ | 28769 | 28785 |
| 2842 | GAAGCCTTTTGGCAATG | NC_045512.2 | 3′→5′ | 28770 | 28786 |
| 2843 | AGAAGCCTTTTGGCAAT | NC_045512.2 | 3′→5′ | 28771 | 28787 |
| 2844 | TAGAAGCCTTTTGGCAA | NC_045512.2 | 3′→5′ | 28772 | 28788 |
| 2845 | GTAGAAGCCTTTTGGCA | NC_045512.2 | 3′→5′ | 28773 | 28789 |
| 2846 | CGTAGAAGCCTTTTGGC | NC_045512.2 | 3′→5′ | 28774 | 28790 |
| 2847 | GCGTAGAAGCCTTTTGG | NC_045512.2 | 3′→5′ | 28775 | 28791 |
| 2848 | TGCGTAGAAGCCTTTTG | NC_045512.2 | 3′→5′ | 28776 | 28792 |
| 2849 | CTGCGTAGAAGCCTTTT | NC_045512.2 | 3′→5′ | 28777 | 28793 |
| 2850 | TCTGCGTAGAAGCCTTT | NC_045512.2 | 3′→5′ | 28778 | 28794 |
| 2851 | TGACTGCCGCCTCTGCT | NC_045512.2 | 3′→5′ | 28799 | 28815 |
| 2852 | TTGACTGCCGCCTCTGC | NC_045512.2 | 3′→5′ | 28800 | 28816 |
| 2853 | CTTGACTGCCGCCTCTG | NC_045512.2 | 3′→5′ | 28801 | 28817 |
| 2854 | GCTTGACTGCCGCCTCT | NC_045512.2 | 3′→5′ | 28802 | 28818 |
| 2855 | GGCTTGACTGCCGCCTC | NC_045512.2 | 3′→5′ | 28803 | 28819 |
| 2856 | AGGCTTGACTGCCGCCT | NC_045512.2 | 3′→5′ | 28804 | 28820 |
| 2857 | GAGGCTTGACTGCCGCC | NC_045512.2 | 3′→5′ | 28805 | 28821 |
| 2858 | AGAGGCTTGACTGCCGC | NC_045512.2 | 3′→5′ | 28806 | 28822 |
| 2859 | AAGAGGCTTGACTGCCG | NC_045512.2 | 3′→5′ | 28807 | 28823 |
| 2860 | GAAGAGGCTTGACTGCC | NC_045512.2 | 3′→5′ | 28808 | 28824 |
| 2861 | AGAAGAGGCTTGACTGC | NC_045512.2 | 3′→5′ | 28809 | 28825 |
| 2862 | GAGAAGAGGCTTGACTG | NC_045512.2 | 3′→5′ | 28810 | 28826 |
| 2863 | CGAGAAGAGGCTTGACT | NC_045512.2 | 3′→5′ | 28811 | 28827 |
| 2864 | GAGTTGAATTTCTTGAA | NC_045512.2 | 3′→5′ | 28852 | 28868 |
| 2865 | GGAGTTGAATTTCTTGA | NC_045512.2 | 3′→5′ | 28853 | 28869 |
| 2866 | AGCTGGTTCAATCTGTC | NC_045512.2 | 3′→5′ | 28946 | 28962 |
| 2867 | AAGCTGGTTCAATCTGT | NC_045512.2 | 3′→5′ | 28947 | 28963 |
| 2868 | CAAGCTGGTTCAATCTG | NC_045512.2 | 3′→5′ | 28948 | 28964 |
| 2869 | TCAAGCTGGTTCAATCT | NC_045512.2 | 3′→5′ | 28949 | 28965 |
| 2870 | CTCAAGCTGGTTCAATC | NC_045512.2 | 3′→5′ | 28950 | 28966 |
| 2871 | TCTCAAGCTGGTTCAAT | NC_045512.2 | 3′→5′ | 28951 | 28967 |
| 2872 | CTCTCAAGCTGGTTCAA | NC_045512.2 | 3′→5′ | 28952 | 28968 |
| 2873 | GCTCTCAAGCTGGTTCA | NC_045512.2 | 3′→5′ | 28953 | 28969 |
| 2874 | TGCTCTCAAGCTGGTTC | NC_045512.2 | 3′→5′ | 28954 | 28970 |
| 2875 | TTGCTCTCAAGCTGGTT | NC_045512.2 | 3′→5′ | 28955 | 28971 |
| 2876 | TTTGCTCTCAAGCTGGT | NC_045512.2 | 3′→5′ | 28956 | 28972 |
| 2877 | TGTTGGCCTTTACCAGA | NC_045512.2 | 3′→5′ | 28976 | 28992 |
| 2878 | TTGTTGGCCTTTACCAG | NC_045512.2 | 3′→5′ | 28977 | 28993 |
| 2879 | GTTGTTGGCCTTTACCA | NC_045512.2 | 3′→5′ | 28978 | 28994 |
| 2880 | TGTTGTTGGCCTTTACC | NC_045512.2 | 3′→5′ | 28979 | 28995 |
| 2881 | TTGTTGTTGGCCTTTAC | NC_045512.2 | 3′→5′ | 28980 | 28996 |
| 2882 | GTTGTTGTTGGCCTTTA | NC_045512.2 | 3′→5′ | 28981 | 28997 |
| 2883 | TGTTGTTGTTGGCCTTT | NC_045512.2 | 3′→5′ | 28982 | 28998 |
| 2884 | TTGTTGTTGTTGGCCTT | NC_045512.2 | 3′→5′ | 28983 | 28999 |
| 2885 | CTTGTTGTTGTTGGCCT | NC_045512.2 | 3′→5′ | 28984 | 29000 |
| 2886 | CCTTGTTGTTGTTGGCC | NC_045512.2 | 3′→5′ | 28985 | 29001 |
| 2887 | GCCTTGTTGTTGTTGGC | NC_045512.2 | 3′→5′ | 28986 | 29002 |
| 2888 | GGCCTTGTTGTTGTTGG | NC_045512.2 | 3′→5′ | 28987 | 29003 |
| 2889 | TGGCCTTGTTGTTGTTG | NC_045512.2 | 3′→5′ | 28988 | 29004 |
| 2890 | TTGGCCTTGTTGTTGTT | NC_045512.2 | 3′→5′ | 28989 | 29005 |
| 2891 | TTTGGCCTTGTTGTTGT | NC_045512.2 | 3′→5′ | 28990 | 29006 |
| 2892 | GTTTGGCCTTGTTGTTG | NC_045512.2 | 3′→5′ | 28991 | 29007 |
| 2893 | AGTTTGGCCTTGTTGTT | NC_045512.2 | 3′→5′ | 28992 | 29008 |
| 2894 | CAGTTTGGCCTTGTTGT | NC_045512.2 | 3′→5′ | 28993 | 29009 |
| 2895 | ACAGTTTGGCCTTGTTG | NC_045512.2 | 3′→5′ | 28994 | 29010 |
| 2896 | GACAGTTTGGCCTTGTT | NC_045512.2 | 3′→5′ | 28995 | 29011 |
| 2897 | TGACAGTTTGGCCTTGT | NC_045512.2 | 3′→5′ | 28996 | 29012 |
| 2898 | GTGACAGTTTGGCCTTG | NC_045512.2 | 3′→5′ | 28997 | 29013 |
| 2899 | AGTGACAGTTTGGCCTT | NC_045512.2 | 3′→5′ | 28998 | 29014 |
| 2900 | TAGTGACAGTTTGGCCT | NC_045512.2 | 3′→5′ | 28999 | 29015 |
| 2901 | TTAGTGACAGTTTGGCC | NC_045512.2 | 3′→5′ | 29000 | 29016 |
| 2902 | CTTAGTGACAGTTTGGC | NC_045512.2 | 3′→5′ | 29001 | 29017 |
| 2903 | TCTTAGTGACAGTTTGG | NC_045512.2 | 3′→5′ | 29002 | 29018 |
| 2904 | TTCTTAGTGACAGTTTG | NC_045512.2 | 3′→5′ | 29003 | 29019 |
| 2905 | TTTCTTAGTGACAGTTT | NC_045512.2 | 3′→5′ | 29004 | 29020 |
| 2906 | ATTTCTTAGTGACAGTT | NC_045512.2 | 3′→5′ | 29005 | 29021 |
| 2907 | GATTTCTTAGTGACAGT | NC_045512.2 | 3′→5′ | 29006 | 29022 |
| 2908 | AGATTTCTTAGTGACAG | NC_045512.2 | 3′→5′ | 29007 | 29023 |
| 2909 | CAGATTTCTTAGTGACA | NC_045512.2 | 3′→5′ | 29008 | 29024 |
| 2910 | GCAGATTTCTTAGTGAC | NC_045512.2 | 3′→5′ | 29009 | 29025 |
| 2911 | AGCAGATTTCTTAGTGA | NC_045512.2 | 3′→5′ | 29010 | 29026 |
| 2912 | CAGCAGATTTCTTAGTG | NC_045512.2 | 3′→5′ | 29011 | 29027 |
| 2913 | GCAGCAGATTTCTTAGT | NC_045512.2 | 3′→5′ | 29012 | 29028 |
| 2914 | AGCAGCAGATTTCTTAG | NC_045512.2 | 3′→5′ | 29013 | 29029 |
| 2915 | CAGCAGCAGATTTCTTA | NC_045512.2 | 3′→5′ | 29014 | 29030 |
| 2916 | TCAGCAGCAGATTTCTT | NC_045512.2 | 3′→5′ | 29015 | 29031 |
| 2917 | CTCAGCAGCAGATTTCT | NC_045512.2 | 3′→5′ | 29016 | 29032 |
| 2918 | CCTCAGCAGCAGATTTC | NC_045512.2 | 3′→5′ | 29017 | 29033 |
| 2919 | GCCTCAGCAGCAGATTT | NC_045512.2 | 3′→5′ | 29018 | 29034 |
| 2920 | GTTCCTTGTCTGATTAG | NC_045512.2 | 3′→5′ | 29144 | 29160 |
| 2921 | AGTTCCTTGTCTGATTA | NC_045512.2 | 3′→5′ | 29145 | 29161 |
| 2922 | CAGTTCCTTGTCTGATT | NC_045512.2 | 3′→5′ | 29146 | 29162 |
| 2923 | TCAGTTCCTTGTCTGAT | NC_045512.2 | 3′→5′ | 29147 | 29163 |
| 2924 | ATCAGTTCCTTGTCTGA | NC_045512.2 | 3′→5′ | 29148 | 29164 |
| 2925 | AATCAGTTCCTTGTCTG | NC_045512.2 | 3′→5′ | 29149 | 29165 |
| 2926 | TAATCAGTTCCTTGTCT | NC_045512.2 | 3′→5′ | 29150 | 29166 |
| 2927 | GTAATCAGTTCCTTGTC | NC_045512.2 | 3′→5′ | 29151 | 29167 |
| 2928 | TGTAATCAGTTCCTTGT | NC_045512.2 | 3′→5′ | 29152 | 29168 |
| 2929 | TTGTAATCAGTTCCTTG | NC_045512.2 | 3′→5′ | 29153 | 29169 |
| 2930 | TTTGTAATCAGTTCCTT | NC_045512.2 | 3′→5′ | 29154 | 29170 |
| 2931 | GTTTGTAATCAGTTCCT | NC_045512.2 | 3′→5′ | 29155 | 29171 |
| 2932 | TGTTTGTAATCAGTTCC | NC_045512.2 | 3′→5′ | 29156 | 29172 |
| 2933 | TGTGCAATTTGCGGCCA | NC_045512.2 | 3′→5′ | 29174 | 29190 |
| 2934 | TTGTGCAATTTGCGGCC | NC_045512.2 | 3′→5′ | 29175 | 29191 |
| 2935 | ATTGTGCAATTTGCGGC | NC_045512.2 | 3′→5′ | 29176 | 29192 |
| 2936 | AATTGTGCAATTTGCGG | NC_045512.2 | 3′→5′ | 29177 | 29193 |
| 2937 | AAATTGTGCAATTTGCG | NC_045512.2 | 3′→5′ | 29178 | 29194 |
| 2938 | CAAATTGTGCAATTTGC | NC_045512.2 | 3′→5′ | 29179 | 29195 |
| 2939 | GCAAATTGTGCAATTTG | NC_045512.2 | 3′→5′ | 29180 | 29196 |
| 2940 | ACTTCCATGCCAATGCG | NC_045512.2 | 3′→5′ | 29228 | 29244 |
| 2941 | GACTTCCATGCCAATGC | NC_045512.2 | 3′→5′ | 29229 | 29245 |
| 2942 | TGACTTCCATGCCAATG | NC_045512.2 | 3′→5′ | 29230 | 29246 |
| 2943 | GTGACTTCCATGCCAAT | NC_045512.2 | 3′→5′ | 29231 | 29247 |
| 2944 | TGTGACTTCCATGCCAA | NC_045512.2 | 3′→5′ | 29232 | 29248 |
| 2945 | GTGTGACTTCCATGCCA | NC_045512.2 | 3′→5′ | 29233 | 29249 |
| 2946 | GGTGTGACTTCCATGCC | NC_045512.2 | 3′→5′ | 29234 | 29250 |
| 2947 | AGGTGTGACTTCCATGC | NC_045512.2 | 3′→5′ | 29235 | 29251 |
| 2948 | AAGGTGTGACTTCCATG | NC_045512.2 | 3′→5′ | 29236 | 29252 |
| 2949 | GAAGGTGTGACTTCCAT | NC_045512.2 | 3′→5′ | 29237 | 29253 |
| 2950 | CGAAGGTGTGACTTCCA | NC_045512.2 | 3′→5′ | 29238 | 29254 |
| 2951 | CCGAAGGTGTGACTTCC | NC_045512.2 | 3′→5′ | 29239 | 29255 |
| 2952 | CCCGAAGGTGTGACTTC | NC_045512.2 | 3′→5′ | 29240 | 29256 |
| 2953 | TCCCGAAGGTGTGACTT | NC_045512.2 | 3′→5′ | 29241 | 29257 |
| 2954 | TTCCCGAAGGTGTGACT | NC_045512.2 | 3′→5′ | 29242 | 29258 |
| 2955 | GTTCCCGAAGGTGTGAC | NC_045512.2 | 3′→5′ | 29243 | 29259 |
| 2956 | TCTTTGTCATCCAATTT | NC_045512.2 | 3′→5′ | 29285 | 29301 |
| 2957 | ATCTTTGTCATCCAATT | NC_045512.2 | 3′→5′ | 29286 | 29302 |
| 2958 | GATCTTTGTCATCCAAT | NC_045512.2 | 3′→5′ | 29287 | 29303 |
| 2959 | GGATCTTTGTCATCCAA | NC_045512.2 | 3′→5′ | 29288 | 29304 |
| 2960 | TGGATCTTTGTCATCCA | NC_045512.2 | 3′→5′ | 29289 | 29305 |
| 2961 | GTTTTGTATGCGTCAAT | NC_045512.2 | 3′→5′ | 29342 | 29358 |
| 2962 | TGTTTTGTATGCGTCAA | NC_045512.2 | 3′→5′ | 29343 | 29359 |
| 2963 | ATGTTTTGTATGCGTCA | NC_045512.2 | 3′→5′ | 29344 | 29360 |
| 2964 | AATGTTTTGTATGCGTC | NC_045512.2 | 3′→5′ | 29345 | 29361 |
| 2965 | GAATGTTTTGTATGCGT | NC_045512.2 | 3′→5′ | 29346 | 29362 |
| 2966 | GGAATGTTTTGTATGCG | NC_045512.2 | 3′→5′ | 29347 | 29363 |
| 2967 | GGGAATGTTTTGTATGC | NC_045512.2 | 3′→5′ | 29348 | 29364 |
| 2968 | TGGGAATGTTTTGTATG | NC_045512.2 | 3′→5′ | 29349 | 29365 |
| 2969 | GTGGGAATGTTTTGTAT | NC_045512.2 | 3′→5′ | 29350 | 29366 |
| 2970 | GGTGGGAATGTTTTGTA | NC_045512.2 | 3′→5′ | 29351 | 29367 |
| 2971 | TGGTGGGAATGTTTTGT | NC_045512.2 | 3′→5′ | 29352 | 29368 |
| 2972 | TTGGTGGGAATGTTTTG | NC_045512.2 | 3′→5′ | 29353 | 29369 |
| 2973 | GTTGGTGGGAATGTTTT | NC_045512.2 | 3′→5′ | 29354 | 29370 |
| 2974 | TGTTGGTGGGAATGTTT | NC_045512.2 | 3′→5′ | 29355 | 29371 |
| 2975 | CTGTTGGTGGGAATGTT | NC_045512.2 | 3′→5′ | 29356 | 29372 |
| 2976 | TCTGTTGGTGGGAATGT | NC_045512.2 | 3′→5′ | 29357 | 29373 |
| 2977 | CTCTGTTGGTGGGAATG | NC_045512.2 | 3′→5′ | 29358 | 29374 |
| 2978 | GCTCTGTTGGTGGGAAT | NC_045512.2 | 3′→5′ | 29359 | 29375 |
| 2979 | GGCTCTGTTGGTGGGAA | NC_045512.2 | 3′→5′ | 29360 | 29376 |
| 2980 | AGGCTCTGTTGGTGGGA | NC_045512.2 | 3′→5′ | 29361 | 29377 |
| 2981 | TAGGCTCTGTTGGTGGG | NC_045512.2 | 3′→5′ | 29362 | 29378 |
| 2982 | TTAGGCTCTGTTGGTGG | NC_045512.2 | 3′→5′ | 29363 | 29379 |
| 2983 | TTTAGGCTCTGTTGGTG | NC_045512.2 | 3′→5′ | 29364 | 29380 |
| 2984 | TTTTAGGCTCTGTTGGT | NC_045512.2 | 3′→5′ | 29365 | 29381 |
| 2985 | TTTTTAGGCTCTGTTGG | NC_045512.2 | 3′→5′ | 29366 | 29382 |
| 2986 | CTTTTTAGGCTCTGTTG | NC_045512.2 | 3′→5′ | 29367 | 29383 |
| 2987 | CCTTTTTAGGCTCTGTT | NC_045512.2 | 3′→5′ | 29368 | 29384 |
| 2988 | TCCTTTTTAGGCTCTGT | NC_045512.2 | 3′→5′ | 29369 | 29385 |
| 2989 | GTCCTTTTTAGGCTCTG | NC_045512.2 | 3′→5′ | 29370 | 29386 |
| 2990 | TGTCCTTTTTAGGCTCT | NC_045512.2 | 3′→5′ | 29371 | 29387 |
| 2991 | TTGTCCTTTTTAGGCTC | NC_045512.2 | 3′→5′ | 29372 | 29388 |
| 2992 | TTTGTCCTTTTTAGGCT | NC_045512.2 | 3′→5′ | 29373 | 29389 |
| 2993 | TTTTGTCCTTTTTAGGC | NC_045512.2 | 3′→5′ | 29374 | 29390 |
| 2994 | TTTTTGTCCTTTTTAGG | NC_045512.2 | 3′→5′ | 29375 | 29391 |
| 2995 | CTTTTTGTCCTTTTTAG | NC_045512.2 | 3′→5′ | 29376 | 29392 |
| 2996 | TCTTTTTGTCCTTTTTA | NC_045512.2 | 3′→5′ | 29377 | 29393 |
| 2997 | TTCTTTTTGTCCTTTTT | NC_045512.2 | 3′→5′ | 29378 | 29394 |
| 2998 | GGAAGAAGAGTCACAGT | NC_045512.2 | 3′→5′ | 29444 | 29460 |
| 2999 | AGGAAGAAGAGTCACAG | NC_045512.2 | 3′→5′ | 29445 | 29461 |
| 3000 | CAGGAAGAAGAGTCACA | NC_045512.2 | 3′→5′ | 29446 | 29462 |
| 3001 | GCAGGAAGAAGAGTCAC | NC_045512.2 | 3′→5′ | 29447 | 29463 |
| 3002 | ATCTGCCTTGTGTGGTC | NC_045512.2 | 3′→5′ | 29543 | 29559 |
| 3003 | CATCTGCCTTGTGTGGT | NC_045512.2 | 3′→5′ | 29544 | 29560 |
| 3004 | CCATCTGCCTTGTGTGG | NC_045512.2 | 3′→5′ | 29545 | 29561 |
| 3005 | CCCATCTGCCTTGTGTG | NC_045512.2 | 3′→5′ | 29546 | 29562 |
| 3006 | GCCCATCTGCCTTGTGT | NC_045512.2 | 3′→5′ | 29547 | 29563 |
| 3007 | AGCCCATCTGCCTTGTG | NC_045512.2 | 3′→5′ | 29548 | 29564 |
| 3008 | TAGCCCATCTGCCTTGT | NC_045512.2 | 3′→5′ | 29549 | 29565 |
| 3009 | ATAGCCCATCTGCCTTG | NC_045512.2 | 3′→5′ | 29550 | 29566 |
| 3010 | GCACAAGAGTAGACTAT | NC_045512.2 | 3′→5′ | 29598 | 29614 |
| 3011 | TGCACAAGAGTAGACTA | NC_045512.2 | 3′→5′ | 29599 | 29615 |
| 3012 | CTGCACAAGAGTAGACT | NC_045512.2 | 3′→5′ | 29600 | 29616 |
| 3013 | TCTGCACAAGAGTAGAC | NC_045512.2 | 3′→5′ | 29601 | 29617 |
| 3014 | TTCTGCACAAGAGTAGA | NC_045512.2 | 3′→5′ | 29602 | 29618 |
| 3015 | ATTCTGCACAAGAGTAG | NC_045512.2 | 3′→5′ | 29603 | 29619 |
| 3016 | CATTCTGCACAAGAGTA | NC_045512.2 | 3′→5′ | 29604 | 29620 |
| 3017 | TCATTCTGCACAAGAGT | NC_045512.2 | 3′→5′ | 29605 | 29621 |
| 3018 | TTCATTCTGCACAAGAG | NC_045512.2 | 3′→5′ | 29606 | 29622 |
| 3019 | ATTCATTCTGCACAAGA | NC_045512.2 | 3′→5′ | 29607 | 29623 |
| 3020 | AATTCATTCTGCACAAG | NC_045512.2 | 3′→5′ | 29608 | 29624 |
| 3021 | GAATTCATTCTGCACAA | NC_045512.2 | 3′→5′ | 29609 | 29625 |
| 3022 | AGAATTCATTCTGCACA | NC_045512.2 | 3′→5′ | 29610 | 29626 |
| 3023 | GAGAATTCATTCTGCAC | NC_045512.2 | 3′→5′ | 29611 | 29627 |
| 3024 | CGAGAATTCATTCTGCA | NC_045512.2 | 3′→5′ | 29612 | 29628 |
| 3025 | ACGAGAATTCATTCTGC | NC_045512.2 | 3′→5′ | 29613 | 29629 |
| 3026 | TACGAGAATTCATTCTG | NC_045512.2 | 3′→5′ | 29614 | 29630 |
| 3027 | GAGATTAAAGTTAACTA | NC_045512.2 | 3′→5′ | 29652 | 29668 |
| 3028 | TGAGATTAAAGTTAACT | NC_045512.2 | 3′→5′ | 29653 | 29669 |
| 3029 | GTGAGATTAAAGTTAAC | NC_045512.2 | 3′→5′ | 29654 | 29670 |
| 3030 | TGTGAGATTAAAGTTAA | NC_045512.2 | 3′→5′ | 29655 | 29671 |
| 3031 | ATGTGAGATTAAAGTTA | NC_045512.2 | 3′→5′ | 29656 | 29672 |
| 3032 | TATGTGAGATTAAAGTT | NC_045512.2 | 3′→5′ | 29657 | 29673 |
| 3033 | CTATGTGAGATTAAAGT | NC_045512.2 | 3′→5′ | 29658 | 29674 |
| 3034 | GCTATGTGAGATTAAAG | NC_045512.2 | 3′→5′ | 29659 | 29675 |
| 3035 | TGCTATGTGAGATTAAA | NC_045512.2 | 3′→5′ | 29660 | 29676 |
| 3036 | TTGCTATGTGAGATTAA | NC_045512.2 | 3′→5′ | 29661 | 29677 |
| 3037 | ATTGCTATGTGAGATTA | NC_045512.2 | 3′→5′ | 29662 | 29678 |
| 3038 | GATTGCTATGTGAGATT | NC_045512.2 | 3′→5′ | 29663 | 29679 |
| 3039 | AGATTGCTATGTGAGAT | NC_045512.2 | 3′→5′ | 29664 | 29680 |
| 3040 | AAGATTGCTATGTGAGA | NC_045512.2 | 3′→5′ | 29665 | 29681 |
| 3041 | AAAGATTGCTATGTGAG | NC_045512.2 | 3′→5′ | 29666 | 29682 |
| 3042 | TAAAGATTGCTATGTGA | NC_045512.2 | 3′→5′ | 29667 | 29683 |
| 3043 | TTAAAGATTGCTATGTG | NC_045512.2 | 3′→5′ | 29668 | 29684 |
| 3044 | ATTAAAGATTGCTATGT | NC_045512.2 | 3′→5′ | 29669 | 29685 |
| 3045 | GATTAAAGATTGCTATG | NC_045512.2 | 3′→5′ | 29670 | 29686 |
| 3046 | TGATTAAAGATTGCTAT | NC_045512.2 | 3′→5′ | 29671 | 29687 |
| 3047 | CTCCCTAATGTTACACA | NC_045512.2 | 3′→5′ | 29689 | 29705 |
| 3048 | CCTCCCTAATGTTACAC | NC_045512.2 | 3′→5′ | 29690 | 29706 |
| 3049 | TCCTCCCTAATGTTACA | NC_045512.2 | 3′→5′ | 29691 | 29707 |
| 3050 | GTCCTCCCTAATGTTAC | NC_045512.2 | 3′→5′ | 29692 | 29708 |
| 3051 | AGTCCTCCCTAATGTTA | NC_045512.2 | 3′→5′ | 29693 | 29709 |
| 3052 | AAGTCCTCCCTAATGTT | NC_045512.2 | 3′→5′ | 29694 | 29710 |
| 3053 | CAAGTCCTCCCTAATGT | NC_045512.2 | 3′→5′ | 29695 | 29711 |
| 3054 | TCAAGTCCTCCCTAATG | NC_045512.2 | 3′→5′ | 29696 | 29712 |
| 3055 | TTCAAGTCCTCCCTAAT | NC_045512.2 | 3′→5′ | 29697 | 29713 |
| 3056 | TTTCAAGTCCTCCCTAA | NC_045512.2 | 3′→5′ | 29698 | 29714 |
| 3057 | CTTTCAAGTCCTCCCTA | NC_045512.2 | 3′→5′ | 29699 | 29715 |
| 3058 | TCTTTCAAGTCCTCCCT | NC_045512.2 | 3′→5′ | 29700 | 29716 |
| 3059 | CTCTTTCAAGTCCTCCC | NC_045512.2 | 3′→5′ | 29701 | 29717 |
| 3060 | GCTCTTTCAAGTCCTCC | NC_045512.2 | 3′→5′ | 29702 | 29718 |
| 3061 | GGCTCTTTCAAGTCCTC | NC_045512.2 | 3′→5′ | 29703 | 29719 |
| 3062 | TGGCTCTTTCAAGTCCT | NC_045512.2 | 3′→5′ | 29704 | 29720 |
| 3063 | GTGGCTCTTTCAAGTCC | NC_045512.2 | 3′→5′ | 29705 | 29721 |
| 3064 | GGTGGCTCTTTCAAGTC | NC_045512.2 | 3′→5′ | 29706 | 29722 |
| 3065 | TGGTGGCTCTTTCAAGT | NC_045512.2 | 3′→5′ | 29707 | 29723 |
| 3066 | GTGGTGGCTCTTTCAAG | NC_045512.2 | 3′→5′ | 29708 | 29724 |
| 3067 | TGTGGTGGCTCTTTCAA | NC_045512.2 | 3′→5′ | 29709 | 29725 |
| 3068 | ATGTGGTGGCTCTTTCA | NC_045512.2 | 3′→5′ | 29710 | 29726 |
| 3069 | AATGTGGTGGCTCTTTC | NC_045512.2 | 3′→5′ | 29711 | 29727 |
| 3070 | AAATGTGGTGGCTCTTT | NC_045512.2 | 3′→5′ | 29712 | 29728 |
| 3071 | AAAATGTGGTGGCTCTT | NC_045512.2 | 3′→5′ | 29713 | 29729 |
| 3072 | GAAAATGTGGTGGCTCT | NC_045512.2 | 3′→5′ | 29714 | 29730 |
| 3073 | TGAAAATGTGGTGGCTC | NC_045512.2 | 3′→5′ | 29715 | 29731 |
| 3074 | TACTCCGCGTGGCCTCG | NC_045512.2 | 3′→5′ | 29733 | 29749 |
| 3075 | GTACTCCGCGTGGCCTC | NC_045512.2 | 3′→5′ | 29734 | 29750 |
| 3076 | CGTACTCCGCGTGGCCT | NC_045512.2 | 3′→5′ | 29735 | 29751 |
| 3077 | TCGTACTCCGCGTGGCC | NC_045512.2 | 3′→5′ | 29736 | 29752 |
| 3078 | ATCGTACTCCGCGTGGC | NC_045512.2 | 3′→5′ | 29737 | 29753 |
| 3079 | GATCGTACTCCGCGTGG | NC_045512.2 | 3′→5′ | 29738 | 29754 |
| 3080 | CGATCGTACTCCGCGTG | NC_045512.2 | 3′→5′ | 29739 | 29755 |
| 3081 | TCGATCGTACTCCGCGT | NC_045512.2 | 3′→5′ | 29740 | 29756 |
| 3082 | CTCGATCGTACTCCGCG | NC_045512.2 | 3′→5′ | 29741 | 29757 |
| 3083 | CAGCTCTCCCTAGCATT | NC_045512.2 | 3′→5′ | 29770 | 29786 |
| 3084 | GCAGCTCTCCCTAGCAT | NC_045512.2 | 3′→5′ | 29771 | 29787 |
| 3085 | GGCAGCTCTCCCTAGCA | NC_045512.2 | 3′→5′ | 29772 | 29788 |
| 3086 | AGGCAGCTCTCCCTAGC | NC_045512.2 | 3′→5′ | 29773 | 29789 |
| 3087 | TAGGCAGCTCTCCCTAG | NC_045512.2 | 3′→5′ | 29774 | 29790 |
| 3088 | ATAGGCAGCTCTCCCTA | NC_045512.2 | 3′→5′ | 29775 | 29791 |
| 3089 | TATAGGCAGCTCTCCCT | NC_045512.2 | 3′→5′ | 29776 | 29792 |
| 3090 | ATATAGGCAGCTCTCCC | NC_045512.2 | 3′→5′ | 29777 | 29793 |
| 3091 | CATATAGGCAGCTCTCC | NC_045512.2 | 3′→5′ | 29778 | 29794 |
| 3092 | CCATATAGGCAGCTCTC | NC_045512.2 | 3′→5′ | 29779 | 29795 |
| 3093 | TCCATATAGGCAGCTCT | NC_045512.2 | 3′→5′ | 29780 | 29796 |
| 3094 | TTCCATATAGGCAGCTC | NC_045512.2 | 3′→5′ | 29781 | 29797 |
| 3095 | CTTCCATATAGGCAGCT | NC_045512.2 | 3′→5′ | 29782 | 29798 |
| 3096 | TCTTCCATATAGGCAGC | NC_045512.2 | 3′→5′ | 29783 | 29799 |
| 3097 | CTCTTCCATATAGGCAG | NC_045512.2 | 3′→5′ | 29784 | 29800 |
| 3098 | GCTCTTCCATATAGGCA | NC_045512.2 | 3′→5′ | 29785 | 29801 |
| 3099 | GGCTCTTCCATATAGGC | NC_045512.2 | 3′→5′ | 29786 | 29802 |
| 3100 | GGGCTCTTCCATATAGG | NC_045512.2 | 3′→5′ | 29787 | 29803 |
| 3101 | AGGGCTCTTCCATATAG | NC_045512.2 | 3′→5′ | 29788 | 29804 |
| 3102 | TAGGGCTCTTCCATATA | NC_045512.2 | 3′→5′ | 29789 | 29805 |
| 3103 | TTAGGGCTCTTCCATAT | NC_045512.2 | 3′→5′ | 29790 | 29806 |
| 3104 | ATTAGGGCTCTTCCATA | NC_045512.2 | 3′→5′ | 29791 | 29807 |
| 3105 | CATTAGGGCTCTTCCAT | NC_045512.2 | 3′→5′ | 29792 | 29808 |
| 3106 | ACATTAGGGCTCTTCCA | NC_045512.2 | 3′→5′ | 29793 | 29809 |
| 3107 | CACATTAGGGCTCTTCC | NC_045512.2 | 3′→5′ | 29794 | 29810 |
| 3108 | ACACATTAGGGCTCTTC | NC_045512.2 | 3′→5′ | 29795 | 29811 |
| 3109 | TACACATTAGGGCTCTT | NC_045512.2 | 3′→5′ | 29796 | 29812 |
| 3110 | TTACACATTAGGGCTCT | NC_045512.2 | 3′→5′ | 29797 | 29813 |
| 3111 | TTTACACATTAGGGCTC | NC_045512.2 | 3′→5′ | 29798 | 29814 |
| 3112 | TTTTACACATTAGGGCT | NC_045512.2 | 3′→5′ | 29799 | 29815 |
| 3113 | ATTTTACACATTAGGGC | NC_045512.2 | 3′→5′ | 29800 | 29816 |
| 3114 | AATTTTACACATTAGGG | NC_045512.2 | 3′→5′ | 29801 | 29817 |
| 3115 | TAATTTTACACATTAGG | NC_045512.2 | 3′→5′ | 29802 | 29818 |
| 3116 | TTAATTTTACACATTAG | NC_045512.2 | 3′→5′ | 29803 | 29819 |
| 3117 | ATTAATTTTACACATTA | NC_045512.2 | 3′→5′ | 29804 | 29820 |
| 3118 | AATTAATTTTACACATT | NC_045512.2 | 3′→5′ | 29805 | 29821 |
| 3119 | AAATTAATTTTACACAT | NC_045512.2 | 3′→5′ | 29806 | 29822 |
| 3120 | AAAATTAATTTTACACA | NC_045512.2 | 3′→5′ | 29807 | 29823 |
| 3121 | TAAAATTAATTTTACAC | NC_045512.2 | 3′→5′ | 29808 | 29824 |
| 3122 | CTAAAATTAATTTTACA | NC_045512.2 | 3′→5′ | 29809 | 29825 |
| 3123 | ACTAAAATTAATTTTAC | NC_045512.2 | 3′→5′ | 29810 | 29826 |
| 3124 | TACTAAAATTAATTTTA | NC_045512.2 | 3′→5′ | 29811 | 29827 |
| 3125 | CTACTAAAATTAATTTT | NC_045512.2 | 3′→5′ | 29812 | 29828 |
| 3126 | GGACGAAACCGTAAGCAG | NC_045512.2 | 3′→5′ | 190 | 207 |
| 3127 | CGGACGAAACCGTAAGCA | NC_045512.2 | 3′→5′ | 191 | 208 |
| 3128 | ACGGACGAAACCGTAAGC | NC_045512.2 | 3′→5′ | 192 | 209 |
| 3129 | CACGGACGAAACCGTAAG | NC_045512.2 | 3′→5′ | 193 | 210 |
| 3130 | ACACGGACGAAACCGTAA | NC_045512.2 | 3′→5′ | 194 | 211 |
| 3131 | AACACGGACGAAACCGTA | NC_045512.2 | 3′→5′ | 195 | 212 |
| 3132 | CAACACGGACGAAACCGT | NC_045512.2 | 3′→5′ | 196 | 213 |
| 3133 | GCAACACGGACGAAACCG | NC_045512.2 | 3′→5′ | 197 | 214 |
| 3134 | TGCAACACGGACGAAACC | NC_045512.2 | 3′→5′ | 198 | 215 |
| 3135 | CTGCAACACGGACGAAAC | NC_045512.2 | 3′→5′ | 199 | 216 |
| 3136 | CACCCGGACGAAACCTAG | NC_045512.2 | 3′→5′ | 233 | 250 |
| 3137 | ACACCCGGACGAAACCTA | NC_045512.2 | 3′→5′ | 234 | 251 |
| 3138 | CACACCCGGACGAAACCT | NC_045512.2 | 3′→5′ | 235 | 252 |
| 3139 | TCACACCCGGACGAAACC | NC_045512.2 | 3′→5′ | 236 | 253 |
| 3140 | GTCACACCCGGACGAAAC | NC_045512.2 | 3′→5′ | 237 | 254 |
| 3141 | GGTCACACCCGGACGAAA | NC_045512.2 | 3′→5′ | 238 | 255 |
| 3142 | CGGTCACACCCGGACGAA | NC_045512.2 | 3′→5′ | 239 | 256 |
| 3143 | TCGGTCACACCCGGACGA | NC_045512.2 | 3′→5′ | 240 | 257 |
| 3144 | TTCGGTCACACCCGGACG | NC_045512.2 | 3′→5′ | 241 | 258 |
| 3145 | TTTCGGTCACACCCGGAC | NC_045512.2 | 3′→5′ | 242 | 259 |
| 3146 | CTTTCGGTCACACCCGGA | NC_045512.2 | 3′→5′ | 243 | 260 |
| 3147 | CCTTTCGGTCACACCCGG | NC_045512.2 | 3′→5′ | 244 | 261 |
| 3148 | ACCTTTCGGTCACACCCG | NC_045512.2 | 3′→5′ | 245 | 262 |
| 3149 | TACCTTTCGGTCACACCC | NC_045512.2 | 3′→5′ | 246 | 263 |
| 3150 | TTACCTTTCGGTCACACC | NC_045512.2 | 3′→5′ | 247 | 264 |
| 3151 | CTTACCTTTCGGTCACAC | NC_045512.2 | 3′→5′ | 248 | 265 |
| 3152 | TCTTACCTTTCGGTCACA | NC_045512.2 | 3′→5′ | 249 | 266 |
| 3153 | ATCTTACCTTTCGGTCAC | NC_045512.2 | 3′→5′ | 250 | 267 |
| 3154 | CATCTTACCTTTCGGTCA | NC_045512.2 | 3′→5′ | 251 | 268 |
| 3155 | CCATCTTACCTTTCGGTC | NC_045512.2 | 3′→5′ | 252 | 269 |
| 3156 | TCCATCTTACCTTTCGGT | NC_045512.2 | 3′→5′ | 253 | 270 |
| 3157 | CTCCATCTTACCTTTCGG | NC_045512.2 | 3′→5′ | 254 | 271 |
| 3158 | TCTCCATCTTACCTTTCG | NC_045512.2 | 3′→5′ | 255 | 272 |
| 3159 | CTCTCCATCTTACCTTTC | NC_045512.2 | 3′→5′ | 256 | 273 |
| 3160 | GCTCTCCATCTTACCTTT | NC_045512.2 | 3′→5′ | 257 | 274 |
| 3161 | GGCTCTCCATCTTACCTT | NC_045512.2 | 3′→5′ | 258 | 275 |
| 3162 | AGGCTCTCCATCTTACCT | NC_045512.2 | 3′→5′ | 259 | 276 |
| 3163 | AAGGCTCTCCATCTTACC | NC_045512.2 | 3′→5′ | 260 | 277 |
| 3164 | CAAGGCTCTCCATCTTAC | NC_045512.2 | 3′→5′ | 261 | 278 |
| 3165 | ACAAGGCTCTCCATCTTA | NC_045512.2 | 3′→5′ | 262 | 279 |
| 3166 | CGTGTGTTTTCTCGTTGA | NC_045512.2 | 3′→5′ | 288 | 305 |
| 3167 | ACGTGTGTTTTCTCGTTG | NC_045512.2 | 3′→5′ | 289 | 306 |
| 3168 | GACGTGTGTTTTCTCGTT | NC_045512.2 | 3′→5′ | 290 | 307 |
| 3169 | GGACGTGTGTTTTCTCGT | NC_045512.2 | 3′→5′ | 291 | 308 |
| 3170 | TGGACGTGTGTTTTCTCG | NC_045512.2 | 3′→5′ | 292 | 309 |
| 3171 | TTGGACGTGTGTTTTCTC | NC_045512.2 | 3′→5′ | 293 | 310 |
| 3172 | GTTGGACGTGTGTTTTCT | NC_045512.2 | 3′→5′ | 294 | 311 |
| 3173 | AGTTGGACGTGTGTTTTC | NC_045512.2 | 3′→5′ | 295 | 312 |
| 3174 | GAGTTGGACGTGTGTTTT | NC_045512.2 | 3′→5′ | 296 | 313 |
| 3175 | TGAGTTGGACGTGTGTTT | NC_045512.2 | 3′→5′ | 297 | 314 |
| 3176 | CTGAGTTGGACGTGTGTT | NC_045512.2 | 3′→5′ | 298 | 315 |
| 3177 | ACTGAGTTGGACGTGTGT | NC_045512.2 | 3′→5′ | 299 | 316 |
| 3178 | AACTGAGTTGGACGTGTG | NC_045512.2 | 3′→5′ | 300 | 317 |
| 3179 | AAACTGAGTTGGACGTGT | NC_045512.2 | 3′→5′ | 301 | 318 |
| 3180 | CAAACTGAGTTGGACGTG | NC_045512.2 | 3′→5′ | 302 | 319 |
| 3181 | GCAAACTGAGTTGGACGT | NC_045512.2 | 3′→5′ | 303 | 320 |
| 3182 | GGCAAACTGAGTTGGACG | NC_045512.2 | 3′→5′ | 304 | 321 |
| 3183 | AGGCAAACTGAGTTGGAC | NC_045512.2 | 3′→5′ | 305 | 322 |
| 3184 | CAGGCAAACTGAGTTGGA | NC_045512.2 | 3′→5′ | 306 | 323 |
| 3185 | ACAGGCAAACTGAGTTGG | NC_045512.2 | 3′→5′ | 307 | 324 |
| 3186 | CACATAGGGCTGTTCAAG | NC_045512.2 | 3′→5′ | 455 | 472 |
| 3187 | ACACATAGGGCTGTTCAA | NC_045512.2 | 3′→5′ | 456 | 473 |
| 3188 | AACACATAGGGCTGTTCA | NC_045512.2 | 3′→5′ | 457 | 474 |
| 3189 | GAACACATAGGGCTGTTC | NC_045512.2 | 3′→5′ | 458 | 475 |
| 3190 | TGAACACATAGGGCTGTT | NC_045512.2 | 3′→5′ | 459 | 476 |
| 3191 | ATGAACACATAGGGCTGT | NC_045512.2 | 3′→5′ | 460 | 477 |
| 3192 | GTTCTTACGAAGAAGAAC | NC_045512.2 | 3′→5′ | 626 | 643 |
| 3193 | CGTTCTTACGAAGAAGAA | NC_045512.2 | 3′→5′ | 627 | 644 |
| 3194 | CCGTTCTTACGAAGAAGA | NC_045512.2 | 3′→5′ | 628 | 645 |
| 3195 | ACCGTTCTTACGAAGAAG | NC_045512.2 | 3′→5′ | 629 | 646 |
| 3196 | TACCGTTCTTACGAAGAA | NC_045512.2 | 3′→5′ | 630 | 647 |
| 3197 | TTACCGTTCTTACGAAGA | NC_045512.2 | 3′→5′ | 631 | 648 |
| 3198 | ATTACCGTTCTTACGAAG | NC_045512.2 | 3′→5′ | 632 | 649 |
| 3199 | TATTACCGTTCTTACGAA | NC_045512.2 | 3′→5′ | 633 | 650 |
| 3200 | TTATTACCGTTCTTACGA | NC_045512.2 | 3′→5′ | 634 | 651 |
| 3201 | ATCAGTGCCAAGCTCGTC | NC_045512.2 | 3′→5′ | 704 | 721 |
| 3202 | GATCAGTGCCAAGCTCGT | NC_045512.2 | 3′→5′ | 705 | 722 |
| 3203 | GGATCAGTGCCAAGCTCG | NC_045512.2 | 3′→5′ | 706 | 723 |
| 3204 | GTAAGTTTTAAATAACCA | NC_045512.2 | 3′→5′ | 3352 | 3369 |
| 3205 | AGTAAGTTTTAAATAACC | NC_045512.2 | 3′→5′ | 3353 | 3370 |
| 3206 | CAGTAAGTTTTAAATAAC | NC_045512.2 | 3′→5′ | 3354 | 3371 |
| 3207 | TCAGTAAGTTTTAAATAA | NC_045512.2 | 3′→5′ | 3355 | 3372 |
| 3208 | GTCAGTAAGTTTTAAATA | NC_045512.2 | 3′→5′ | 3356 | 3373 |
| 3209 | TGTCAGTAAGTTTTAAAT | NC_045512.2 | 3′→5′ | 3357 | 3374 |
| 3210 | TTGTCAGTAAGTTTTAAA | NC_045512.2 | 3′→5′ | 3358 | 3375 |
| 3211 | ATTGTCAGTAAGTTTTAA | NC_045512.2 | 3′→5′ | 3359 | 3376 |
| 3212 | CATTGTCAGTAAGTTTTA | NC_045512.2 | 3′→5′ | 3360 | 3377 |
| 3213 | ACATTGTCAGTAAGTTTT | NC_045512.2 | 3′→5′ | 3361 | 3378 |
| 3214 | TGCACAAAAGTTAGCAGC | NC_045512.2 | 3′→5′ | 5384 | 5401 |
| 3215 | GTGCACAAAAGTTAGCAG | NC_045512.2 | 3′→5′ | 5385 | 5402 |
| 3216 | AGTGCACAAAAGTTAGCA | NC_045512.2 | 3′→5′ | 5386 | 5403 |
| 3217 | TCCACTACTTCTTCAGAG | NC_045512.2 | 3′→5′ | 6406 | 6423 |
| 3218 | TTCCACTACTTCTTCAGA | NC_045512.2 | 3′→5′ | 6407 | 6424 |
| 3219 | TTTCCACTACTTCTTCAG | NC_045512.2 | 3′→5′ | 6408 | 6425 |
| 3220 | TTTTCCACTACTTCTTCA | NC_045512.2 | 3′→5′ | 6409 | 6426 |
| 3221 | ATTTTCCACTACTTCTTC | NC_045512.2 | 3′→5′ | 6410 | 6427 |
| 3222 | GATTTTCCACTACTTCTT | NC_045512.2 | 3′→5′ | 6411 | 6428 |
| 3223 | GGATTTTCCACTACTTCT | NC_045512.2 | 3′→5′ | 6412 | 6429 |
| 3224 | AGGATTTTCCACTACTTC | NC_045512.2 | 3′→5′ | 6413 | 6430 |
| 3225 | TAGGATTTTCCACTACTT | NC_045512.2 | 3′→5′ | 6414 | 6431 |
| 3226 | GTAGGATTTTCCACTACT | NC_045512.2 | 3′→5′ | 6415 | 6432 |
| 3227 | GGTAGGATTTTCCACTAC | NC_045512.2 | 3′→5′ | 6416 | 6433 |
| 3228 | AACTTCGGTAGTTTTCAC | NC_045512.2 | 3′→5′ | 6461 | 6478 |
| 3229 | CAACTTCGGTAGTTTTCA | NC_045512.2 | 3′→5′ | 6462 | 6479 |
| 3230 | ACAACTTCGGTAGTTTTC | NC_045512.2 | 3′→5′ | 6463 | 6480 |
| 3231 | TACAACTTCGGTAGTTTT | NC_045512.2 | 3′→5′ | 6464 | 6481 |
| 3232 | CTACAACTTCGGTAGTTT | NC_045512.2 | 3′→5′ | 6465 | 6482 |
| 3233 | CCTACAACTTCGGTAGTT | NC_045512.2 | 3′→5′ | 6466 | 6483 |
| 3234 | ATTAACAATAGTTGTACA | NC_045512.2 | 3′→5′ | 7532 | 7549 |
| 3235 | CATTAACAATAGTTGTAC | NC_045512.2 | 3′→5′ | 7533 | 7550 |
| 3236 | CCATTAACAATAGTTGTA | NC_045512.2 | 3′→5′ | 7534 | 7551 |
| 3237 | ATGTCACGAGTGACACCA | NC_045512.2 | 3′→5′ | 8707 | 8724 |
| 3238 | ATGTCAAGTACAAGTAAA | NC_045512.2 | 3′→5′ | 9588 | 9605 |
| 3239 | AATGTCAAGTACAAGTAA | NC_045512.2 | 3′→5′ | 9589 | 9606 |
| 3240 | ACCAACACTACCACATGA | NC_045512.2 | 3′→5′ | 10484 | 10501 |
| 3241 | AACCAACACTACCACATG | NC_045512.2 | 3′→5′ | 10485 | 10502 |
| 3242 | AAACCAACACTACCACAT | NC_045512.2 | 3′→5′ | 10486 | 10503 |
| 3243 | AAAACCAACACTACCACA | NC_045512.2 | 3′→5′ | 10487 | 10504 |
| 3244 | TAAAACCAACACTACCAC | NC_045512.2 | 3′→5′ | 10488 | 10505 |
| 3245 | TTAAAACCAACACTACCA | NC_045512.2 | 3′→5′ | 10489 | 10506 |
| 3246 | GTTAAAACCAACACTACC | NC_045512.2 | 3′→5′ | 10490 | 10507 |
| 3247 | TGTTAAAACCAACACTAC | NC_045512.2 | 3′→5′ | 10491 | 10508 |
| 3248 | ATGTTAAAACCAACACTA | NC_045512.2 | 3′→5′ | 10492 | 10509 |
| 3249 | GCCTAAGAAACAATAAAC | NC_045512.2 | 3′→5′ | 11609 | 11626 |
| 3250 | AGCCTAAGAAACAATAAA | NC_045512.2 | 3′→5′ | 11610 | 11627 |
| 3251 | TAGCCTAAGAAACAATAA | NC_045512.2 | 3′→5′ | 11611 | 11628 |
| 3252 | ATAGCCTAAGAAACAATA | NC_045512.2 | 3′→5′ | 11612 | 11629 |
| 3253 | AATAGCCTAAGAAACAAT | NC_045512.2 | 3′→5′ | 11613 | 11630 |
| 3254 | CATTTTAGACTGTACAGT | NC_045512.2 | 3′→5′ | 11834 | 11851 |
| 3255 | ACATTTTAGACTGTACAG | NC_045512.2 | 3′→5′ | 11835 | 11852 |
| 3256 | GACATTTTAGACTGTACA | NC_045512.2 | 3′→5′ | 11836 | 11853 |
| 3257 | TACAGCACCCTGCATGGA | NC_045512.2 | 3′→5′ | 12023 | 12040 |
| 3258 | CTACAGCACCCTGCATGG | NC_045512.2 | 3′→5′ | 12024 | 12041 |
| 3259 | TCTACAGCACCCTGCATG | NC_045512.2 | 3′→5′ | 12025 | 12042 |
| 3260 | GTCTACAGCACCCTGCAT | NC_045512.2 | 3′→5′ | 12026 | 12043 |
| 3261 | TGTCTACAGCACCCTGCA | NC_045512.2 | 3′→5′ | 12027 | 12044 |
| 3262 | ATGTCTACAGCACCCTGC | NC_045512.2 | 3′→5′ | 12028 | 12045 |
| 3263 | TTTAGCCACATTCAAAGA | NC_045512.2 | 3′→5′ | 12212 | 12229 |
| 3264 | ATTTAGCCACATTCAAAG | NC_045512.2 | 3′→5′ | 12213 | 12230 |
| 3265 | GATTTAGCCACATTCAAA | NC_045512.2 | 3′→5′ | 12214 | 12231 |
| 3266 | AGATTTAGCCACATTCAA | NC_045512.2 | 3′→5′ | 12215 | 12232 |
| 3267 | CAGATTTAGCCACATTCA | NC_045512.2 | 3′→5′ | 12216 | 12233 |
| 3268 | TCAGATTTAGCCACATTC | NC_045512.2 | 3′→5′ | 12217 | 12234 |
| 3269 | ATTGTTGATAATGTTGTT | NC_045512.2 | 3′→5′ | 12401 | 12418 |
| 3270 | CATTGTTGATAATGTTGT | NC_045512.2 | 3′→5′ | 12402 | 12419 |
| 3271 | GCATTGTTGATAATGTTG | NC_045512.2 | 3′→5′ | 12403 | 12420 |
| 3272 | AGGGAATCTAGCCCATTT | NC_045512.2 | 3′→5′ | 12839 | 12856 |
| 3273 | TAGGGAATCTAGCCCATT | NC_045512.2 | 3′→5′ | 12840 | 12857 |
| 3274 | TTAGGGAATCTAGCCCAT | NC_045512.2 | 3′→5′ | 12841 | 12858 |
| 3275 | CTTAGGGAATCTAGCCCA | NC_045512.2 | 3′→5′ | 12842 | 12859 |
| 3276 | TCTTAGGGAATCTAGCCC | NC_045512.2 | 3′→5′ | 12843 | 12860 |
| 3277 | CTCTTAGGGAATCTAGCC | NC_045512.2 | 3′→5′ | 12844 | 12861 |
| 3278 | ACTCTTAGGGAATCTAGC | NC_045512.2 | 3′→5′ | 12845 | 12862 |
| 3279 | CACTCTTAGGGAATCTAG | NC_045512.2 | 3′→5′ | 12846 | 12863 |
| 3280 | TCACTCTTAGGGAATCTA | NC_045512.2 | 3′→5′ | 12847 | 12864 |
| 3281 | ATCACTCTTAGGGAATCT | NC_045512.2 | 3′→5′ | 12848 | 12865 |
| 3282 | CATCACTCTTAGGGAATC | NC_045512.2 | 3′→5′ | 12849 | 12866 |
| 3283 | CCATCACTCTTAGGGAAT | NC_045512.2 | 3′→5′ | 12850 | 12867 |
| 3284 | AAGGTGGTTCCAGTTCTG | NC_045512.2 | 3′→5′ | 12885 | 12902 |
| 3285 | CAAGGTGGTTCCAGTTCT | NC_045512.2 | 3′→5′ | 12886 | 12903 |
| 3286 | ACAAGGTGGTTCCAGTTC | NC_045512.2 | 3′→5′ | 12887 | 12904 |
| 3287 | TACAAGGTGGTTCCAGTT | NC_045512.2 | 3′→5′ | 12888 | 12905 |
| 3288 | CTACAAGGTGGTTCCAGT | NC_045512.2 | 3′→5′ | 12889 | 12906 |
| 3289 | CCTACAAGGTGGTTCCAG | NC_045512.2 | 3′→5′ | 12890 | 12907 |
| 3290 | ACCTACAAGGTGGTTCCA | NC_045512.2 | 3′→5′ | 12891 | 12908 |
| 3291 | AACCTACAAGGTGGTTCC | NC_045512.2 | 3′→5′ | 12892 | 12909 |
| 3292 | AAACCTACAAGGTGGTTC | NC_045512.2 | 3′→5′ | 12893 | 12910 |
| 3293 | CAAACCTACAAGGTGGTT | NC_045512.2 | 3′→5′ | 12894 | 12911 |
| 3294 | ACAAACCTACAAGGTGGT | NC_045512.2 | 3′→5′ | 12895 | 12912 |
| 3295 | AACAAACCTACAAGGTGG | NC_045512.2 | 3′→5′ | 12896 | 12913 |
| 3296 | TAACAAACCTACAAGGTG | NC_045512.2 | 3′→5′ | 12897 | 12914 |
| 3297 | GTAACAAACCTACAAGGT | NC_045512.2 | 3′→5′ | 12898 | 12915 |
| 3298 | TGTAACAAACCTACAAGG | NC_045512.2 | 3′→5′ | 12899 | 12916 |
| 3299 | CTGTAACAAACCTACAAG | NC_045512.2 | 3′→5′ | 12900 | 12917 |
| 3300 | TCTGTAACAAACCTACAA | NC_045512.2 | 3′→5′ | 12901 | 12918 |
| 3301 | GTCTGTAACAAACCTACA | NC_045512.2 | 3′→5′ | 12902 | 12919 |
| 3302 | TGTCTGTAACAAACCTAC | NC_045512.2 | 3′→5′ | 12903 | 12920 |
| 3303 | GTGTCTGTAACAAACCTA | NC_045512.2 | 3′→5′ | 12904 | 12921 |
| 3304 | TGTGTCTGTAACAAACCT | NC_045512.2 | 3′→5′ | 12905 | 12922 |
| 3305 | GTGTGTCTGTAACAAACC | NC_045512.2 | 3′→5′ | 12906 | 12923 |
| 3306 | GGTGTGTCTGTAACAAAC | NC_045512.2 | 3′→5′ | 12907 | 12924 |
| 3307 | CTCTATTTAGGTTGTTTA | NC_045512.2 | 3′→5′ | 12966 | 12983 |
| 3308 | CCTCTATTTAGGTTGTTT | NC_045512.2 | 3′→5′ | 12967 | 12984 |
| 3309 | ACCTCTATTTAGGTTGTT | NC_045512.2 | 3′→5′ | 12968 | 12985 |
| 3310 | TACCTCTATTTAGGTTGT | NC_045512.2 | 3′→5′ | 12969 | 12986 |
| 3311 | ATACCTCTATTTAGGTTG | NC_045512.2 | 3′→5′ | 12970 | 12987 |
| 3312 | CATACCTCTATTTAGGTT | NC_045512.2 | 3′→5′ | 12971 | 12988 |
| 3313 | CCATACCTCTATTTAGGT | NC_045512.2 | 3′→5′ | 12972 | 12989 |
| 3314 | ACCATACCTCTATTTAGG | NC_045512.2 | 3′→5′ | 12973 | 12990 |
| 3315 | GTGTGTACACAACATCTT | NC_045512.2 | 3′→5′ | 13151 | 13168 |
| 3316 | TGTGTGTACACAACATCT | NC_045512.2 | 3′→5′ | 13152 | 13169 |
| 3317 | GTGTGTGTACACAACATC | NC_045512.2 | 3′→5′ | 13153 | 13170 |
| 3318 | AGTGTGTGTACACAACAT | NC_045512.2 | 3′→5′ | 13154 | 13171 |
| 3319 | CAGTGTGTGTACACAACA | NC_045512.2 | 3′→5′ | 13155 | 13172 |
| 3320 | CCAGTGTGTGTACACAAC | NC_045512.2 | 3′→5′ | 13156 | 13173 |
| 3321 | ACCAGTGTGTGTACACAA | NC_045512.2 | 3′→5′ | 13157 | 13174 |
| 3322 | TACCAGTGTGTGTACACA | NC_045512.2 | 3′→5′ | 13158 | 13175 |
| 3323 | GTACCAGTGTGTGTACAC | NC_045512.2 | 3′→5′ | 13159 | 13176 |
| 3324 | ACGGTACAGACTGTGTTT | NC_045512.2 | 3′→5′ | 13363 | 13380 |
| 3325 | GACGGTACAGACTGTGTT | NC_045512.2 | 3′→5′ | 13364 | 13381 |
| 3326 | AGACGGTACAGACTGTGT | NC_045512.2 | 3′→5′ | 13365 | 13382 |
| 3327 | CAGACGGTACAGACTGTG | NC_045512.2 | 3′→5′ | 13366 | 13383 |
| 3328 | GCAGACGGTACAGACTGT | NC_045512.2 | 3′→5′ | 13367 | 13384 |
| 3329 | CGCAGACGGTACAGACTG | NC_045512.2 | 3′→5′ | 13368 | 13385 |
| 3330 | CCGCAGACGGTACAGACT | NC_045512.2 | 3′→5′ | 13369 | 13386 |
| 3331 | GCCATAACCTTTCCACAT | NC_045512.2 | 3′→5′ | 13388 | 13405 |
| 3332 | AGCCATAACCTTTCCACA | NC_045512.2 | 3′→5′ | 13389 | 13406 |
| 3333 | CAGCCATAACCTTTCCAC | NC_045512.2 | 3′→5′ | 13390 | 13407 |
| 3334 | ACAGCCATAACCTTTCCA | NC_045512.2 | 3′→5′ | 13391 | 13408 |
| 3335 | TACAGCCATAACCTTTCC | NC_045512.2 | 3′→5′ | 13392 | 13409 |
| 3336 | CTACAGCCATAACCTTTC | NC_045512.2 | 3′→5′ | 13393 | 13410 |
| 3337 | ACTACAGCCATAACCTTT | NC_045512.2 | 3′→5′ | 13394 | 13411 |
| 3338 | AACTACAGCCATAACCTT | NC_045512.2 | 3′→5′ | 13395 | 13412 |
| 3339 | CAACTACAGCCATAACCT | NC_045512.2 | 3′→5′ | 13396 | 13413 |
| 3340 | ACAACTACAGCCATAACC | NC_045512.2 | 3′→5′ | 13397 | 13414 |
| 3341 | CACAACTACAGCCATAAC | NC_045512.2 | 3′→5′ | 13398 | 13415 |
| 3342 | TCACAACTACAGCCATAA | NC_045512.2 | 3′→5′ | 13399 | 13416 |
| 3343 | CAAACCCGTTTAAAAACG | NC_045512.2 | 3′→5′ | 13458 | 13475 |
| 3344 | GCAAACCCGTTTAAAAAC | NC_045512.2 | 3′→5′ | 13459 | 13476 |
| 3345 | CGCAAACCCGTTTAAAAA | NC_045512.2 | 3′→5′ | 13460 | 13477 |
| 3346 | CCGCAAACCCGTTTAAAA | NC_045512.2 | 3′→5′ | 13461 | 13478 |
| 3347 | ACCGCAAACCCGTTTAAA | NC_045512.2 | 3′→5′ | 13462 | 13479 |
| 3348 | CACCGCAAACCCGTTTAA | NC_045512.2 | 3′→5′ | 13463 | 13480 |
| 3349 | ACACCGCAAACCCGTTTA | NC_045512.2 | 3′→5′ | 13464 | 13481 |
| 3350 | TACACCGCAAACCCGTTT | NC_045512.2 | 3′→5′ | 13465 | 13482 |
| 3351 | TTACACCGCAAACCCGTT | NC_045512.2 | 3′→5′ | 13466 | 13483 |
| 3352 | CTTACACCGCAAACCCGT | NC_045512.2 | 3′→5′ | 13467 | 13484 |
| 3353 | ACTTACACCGCAAACCCG | NC_045512.2 | 3′→5′ | 13468 | 13485 |
| 3354 | CACTTACACCGCAAACCC | NC_045512.2 | 3′→5′ | 13469 | 13486 |
| 3355 | GCACTTACACCGCAAACC | NC_045512.2 | 3′→5′ | 13470 | 13487 |
| 3356 | TGCACTTACACCGCAAAC | NC_045512.2 | 3′→5′ | 13471 | 13488 |
| 3357 | CTGCACTTACACCGCAAA | NC_045512.2 | 3′→5′ | 13472 | 13489 |
| 3358 | GCTGCACTTACACCGCAA | NC_045512.2 | 3′→5′ | 13473 | 13490 |
| 3359 | GGCTGCACTTACACCGCA | NC_045512.2 | 3′→5′ | 13474 | 13491 |
| 3360 | GGGCTGCACTTACACCGC | NC_045512.2 | 3′→5′ | 13475 | 13492 |
| 3361 | CGGGCTGCACTTACACCG | NC_045512.2 | 3′→5′ | 13476 | 13493 |
| 3362 | ACGGGCTGCACTTACACC | NC_045512.2 | 3′→5′ | 13477 | 13494 |
| 3363 | GACGGGCTGCACTTACAC | NC_045512.2 | 3′→5′ | 13478 | 13495 |
| 3364 | AGACGGGCTGCACTTACA | NC_045512.2 | 3′→5′ | 13479 | 13496 |
| 3365 | AAGACGGGCTGCACTTAC | NC_045512.2 | 3′→5′ | 13480 | 13497 |
| 3366 | TAAGACGGGCTGCACTTA | NC_045512.2 | 3′→5′ | 13481 | 13498 |
| 3367 | GTAAGACGGGCTGCACTT | NC_045512.2 | 3′→5′ | 13482 | 13499 |
| 3368 | TGTAAGACGGGCTGCACT | NC_045512.2 | 3′→5′ | 13483 | 13500 |
| 3369 | GTGTAAGACGGGCTGCAC | NC_045512.2 | 3′→5′ | 13484 | 13501 |
| 3370 | GGTGTAAGACGGGCTGCA | NC_045512.2 | 3′→5′ | 13485 | 13502 |
| 3371 | CGGTGTAAGACGGGCTGC | NC_045512.2 | 3′→5′ | 13486 | 13503 |
| 3372 | ACGGTGTAAGACGGGCTG | NC_045512.2 | 3′→5′ | 13487 | 13504 |
| 3373 | CACGGTGTAAGACGGGCT | NC_045512.2 | 3′→5′ | 13488 | 13505 |
| 3374 | GCACGGTGTAAGACGGGC | NC_045512.2 | 3′→5′ | 13489 | 13506 |
| 3375 | CGCACGGTGTAAGACGGG | NC_045512.2 | 3′→5′ | 13490 | 13507 |
| 3376 | CCGCACGGTGTAAGACGG | NC_045512.2 | 3′→5′ | 13491 | 13508 |
| 3377 | GCCGCACGGTGTAAGACG | NC_045512.2 | 3′→5′ | 13492 | 13509 |
| 3378 | TGCCGCACGGTGTAAGAC | NC_045512.2 | 3′→5′ | 13493 | 13510 |
| 3379 | GTGCCGCACGGTGTAAGA | NC_045512.2 | 3′→5′ | 13494 | 13511 |
| 3380 | TGTGCCGCACGGTGTAAG | NC_045512.2 | 3′→5′ | 13495 | 13512 |
| 3381 | CTGTGCCGCACGGTGTAA | NC_045512.2 | 3′→5′ | 13496 | 13513 |
| 3382 | CCTGTGCCGCACGGTGTA | NC_045512.2 | 3′→5′ | 13497 | 13514 |
| 3383 | GCCTGTGCCGCACGGTGT | NC_045512.2 | 3′→5′ | 13498 | 13515 |
| 3384 | TGCCTGTGCCGCACGGTG | NC_045512.2 | 3′→5′ | 13499 | 13516 |
| 3385 | GTGCCTGTGCCGCACGGT | NC_045512.2 | 3′→5′ | 13500 | 13517 |
| 3386 | AGTGCCTGTGCCGCACGG | NC_045512.2 | 3′→5′ | 13501 | 13518 |
| 3387 | TAGTGCCTGTGCCGCACG | NC_045512.2 | 3′→5′ | 13502 | 13519 |
| 3388 | CTAGTGCCTGTGCCGCAC | NC_045512.2 | 3′→5′ | 13503 | 13520 |
| 3389 | ATGTGGTACCATGTCACC | NC_045512.2 | 3′→5′ | 13762 | 13779 |
| 3390 | TATGTGGTACCATGTCAC | NC_045512.2 | 3′→5′ | 13763 | 13780 |
| 3391 | ATATGTGGTACCATGTCA | NC_045512.2 | 3′→5′ | 13764 | 13781 |
| 3392 | TATATGTGGTACCATGTC | NC_045512.2 | 3′→5′ | 13765 | 13782 |
| 3393 | ATATATGTGGTACCATGT | NC_045512.2 | 3′→5′ | 13766 | 13783 |
| 3394 | GATATATGTGGTACCATG | NC_045512.2 | 3′→5′ | 13767 | 13784 |
| 3395 | TGATATATGTGGTACCAT | NC_045512.2 | 3′→5′ | 13768 | 13785 |
| 3396 | GTGATATATGTGGTACCA | NC_045512.2 | 3′→5′ | 13769 | 13786 |
| 3397 | CGTGATATATGTGGTACC | NC_045512.2 | 3′→5′ | 13770 | 13787 |
| 3398 | ACGTGATATATGTGGTAC | NC_045512.2 | 3′→5′ | 13771 | 13788 |
| 3399 | GACGTGATATATGTGGTA | NC_045512.2 | 3′→5′ | 13772 | 13789 |
| 3400 | TGACGTGATATATGTGGT | NC_045512.2 | 3′→5′ | 13773 | 13790 |
| 3401 | ATATTTAAAATAACGGTC | NC_045512.2 | 3′→5′ | 14290 | 14307 |
| 3402 | AATATTTAAAATAACGGT | NC_045512.2 | 3′→5′ | 14291 | 14308 |
| 3403 | CAATATTTAAAATAACGG | NC_045512.2 | 3′→5′ | 14292 | 14309 |
| 3404 | CCAATATTTAAAATAACG | NC_045512.2 | 3′→5′ | 14293 | 14310 |
| 3405 | CCCAATATTTAAAATAAC | NC_045512.2 | 3′→5′ | 14294 | 14311 |
| 3406 | TCCCAATATTTAAAATAA | NC_045512.2 | 3′→5′ | 14295 | 14312 |
| 3407 | TCCAAAACTTGTAGGTGG | NC_045512.2 | 3′→5′ | 14404 | 14421 |
| 3408 | GTCCAAAACTTGTAGGTG | NC_045512.2 | 3′→5′ | 14405 | 14422 |
| 3409 | GGTCCAAAACTTGTAGGT | NC_045512.2 | 3′→5′ | 14406 | 14423 |
| 3410 | TGGTCCAAAACTTGTAGG | NC_045512.2 | 3′→5′ | 14407 | 14424 |
| 3411 | GTGGTCCAAAACTTGTAG | NC_045512.2 | 3′→5′ | 14408 | 14425 |
| 3412 | AGTGGTCCAAAACTTGTA | NC_045512.2 | 3′→5′ | 14409 | 14426 |
| 3413 | TAGTGGTCCAAAACTTGT | NC_045512.2 | 3′→5′ | 14410 | 14427 |
| 3414 | CTAGTGGTCCAAAACTTG | NC_045512.2 | 3′→5′ | 14411 | 14428 |
| 3415 | ACTAGTGGTCCAAAACTT | NC_045512.2 | 3′→5′ | 14412 | 14429 |
| 3416 | TACATCCTGATTATGTAC | NC_045512.2 | 3′→5′ | 14500 | 14517 |
| 3417 | TTACATCCTGATTATGTA | NC_045512.2 | 3′→5′ | 14501 | 14518 |
| 3418 | TTTACATCCTGATTATGT | NC_045512.2 | 3′→5′ | 14502 | 14519 |
| 3419 | GTTTACATCCTGATTATG | NC_045512.2 | 3′→5′ | 14503 | 14520 |
| 3420 | AGTTTACATCCTGATTAT | NC_045512.2 | 3′→5′ | 14504 | 14521 |
| 3421 | AAGTTTACATCCTGATTA | NC_045512.2 | 3′→5′ | 14505 | 14522 |
| 3422 | TAAGTTTACATCCTGATT | NC_045512.2 | 3′→5′ | 14506 | 14523 |
| 3423 | GTAAGTTTACATCCTGAT | NC_045512.2 | 3′→5′ | 14507 | 14524 |
| 3424 | TGTAAGTTTACATCCTGA | NC_045512.2 | 3′→5′ | 14508 | 14525 |
| 3425 | ATGTAAGTTTACATCCTG | NC_045512.2 | 3′→5′ | 14509 | 14526 |
| 3426 | TATGTAAGTTTACATCCT | NC_045512.2 | 3′→5′ | 14510 | 14527 |
| 3427 | CTATGTAAGTTTACATCC | NC_045512.2 | 3′→5′ | 14511 | 14528 |
| 3428 | GCTATGTAAGTTTACATC | NC_045512.2 | 3′→5′ | 14512 | 14529 |
| 3429 | AGCTATGTAAGTTTACAT | NC_045512.2 | 3′→5′ | 14513 | 14530 |
| 3430 | GAGCTATGTAAGTTTACA | NC_045512.2 | 3′→5′ | 14514 | 14531 |
| 3431 | TGCAGCTACTGAAAAGCA | NC_045512.2 | 3′→5′ | 14623 | 14640 |
| 3432 | GTGCAGCTACTGAAAAGC | NC_045512.2 | 3′→5′ | 14624 | 14641 |
| 3433 | AGTGCAGCTACTGAAAAG | NC_045512.2 | 3′→5′ | 14625 | 14642 |
| 3434 | AGTTTGAAAAGCAACATT | NC_045512.2 | 3′→5′ | 14650 | 14667 |
| 3435 | CAGTTTGAAAAGCAACAT | NC_045512.2 | 3′→5′ | 14651 | 14668 |
| 3436 | ACAGTTTGAAAAGCAACA | NC_045512.2 | 3′→5′ | 14652 | 14669 |
| 3437 | GACAGTTTGAAAAGCAAC | NC_045512.2 | 3′→5′ | 14653 | 14670 |
| 3438 | TGACAGTTTGAAAAGCAA | NC_045512.2 | 3′→5′ | 14654 | 14671 |
| 3439 | TTGACAGTTTGAAAAGCA | NC_045512.2 | 3′→5′ | 14655 | 14672 |
| 3440 | TTTGACAGTTTGAAAAGC | NC_045512.2 | 3′→5′ | 14656 | 14673 |
| 3441 | GTTTGACAGTTTGAAAAG | NC_045512.2 | 3′→5′ | 14657 | 14674 |
| 3442 | GGTTTGACAGTTTGAAAA | NC_045512.2 | 3′→5′ | 14658 | 14675 |
| 3443 | GGGTTTGACAGTTTGAAA | NC_045512.2 | 3′→5′ | 14659 | 14676 |
| 3444 | CGGGTTTGACAGTTTGAA | NC_045512.2 | 3′→5′ | 14660 | 14677 |
| 3445 | CCGGGTTTGACAGTTTGA | NC_045512.2 | 3′→5′ | 14661 | 14678 |
| 3446 | ACCGGGTTTGACAGTTTG | NC_045512.2 | 3′→5′ | 14662 | 14679 |
| 3447 | TACCGGGTTTGACAGTTT | NC_045512.2 | 3′→5′ | 14663 | 14680 |
| 3448 | TTACCGGGTTTGACAGTT | NC_045512.2 | 3′→5′ | 14664 | 14681 |
| 3449 | ATTACCGGGTTTGACAGT | NC_045512.2 | 3′→5′ | 14665 | 14682 |
| 3450 | AATTACCGGGTTTGACAG | NC_045512.2 | 3′→5′ | 14666 | 14683 |
| 3451 | AAATTACCGGGTTTGACA | NC_045512.2 | 3′→5′ | 14667 | 14684 |
| 3452 | AAAATTACCGGGTTTGAC | NC_045512.2 | 3′→5′ | 14668 | 14685 |
| 3453 | TAAAATTACCGGGTTTGA | NC_045512.2 | 3′→5′ | 14669 | 14686 |
| 3454 | TTAAAATTACCGGGTTTG | NC_045512.2 | 3′→5′ | 14670 | 14687 |
| 3455 | AGACACAGCAAAGTCATA | NC_045512.2 | 3′→5′ | 14698 | 14715 |
| 3456 | TAGACACAGCAAAGTCAT | NC_045512.2 | 3′→5′ | 14699 | 14716 |
| 3457 | TTAGACACAGCAAAGTCA | NC_045512.2 | 3′→5′ | 14700 | 14717 |
| 3458 | ACTTCCTTCCTTAAAGAA | NC_045512.2 | 3′→5′ | 14722 | 14739 |
| 3459 | AACTTCCTTCCTTAAAGA | NC_045512.2 | 3′→5′ | 14723 | 14740 |
| 3460 | GAACTTCCTTCCTTAAAG | NC_045512.2 | 3′→5′ | 14724 | 14741 |
| 3461 | AGAACTTCCTTCCTTAAA | NC_045512.2 | 3′→5′ | 14725 | 14742 |
| 3462 | CAGAACTTCCTTCCTTAA | NC_045512.2 | 3′→5′ | 14726 | 14743 |
| 3463 | ACAGAACTTCCTTCCTTA | NC_045512.2 | 3′→5′ | 14727 | 14744 |
| 3464 | AACAGAACTTCCTTCCTT | NC_045512.2 | 3′→5′ | 14728 | 14745 |
| 3465 | CAACAGAACTTCCTTCCT | NC_045512.2 | 3′→5′ | 14729 | 14746 |
| 3466 | TCAACAGAACTTCCTTCC | NC_045512.2 | 3′→5′ | 14730 | 14747 |
| 3467 | TTCAACAGAACTTCCTTC | NC_045512.2 | 3′→5′ | 14731 | 14748 |
| 3468 | CAAAGAAGAAGTGTTTTA | NC_045512.2 | 3′→5′ | 14750 | 14767 |
| 3469 | GCAAAGAAGAAGTGTTTT | NC_045512.2 | 3′→5′ | 14751 | 14768 |
| 3470 | AGCAAAGAAGAAGTGTTT | NC_045512.2 | 3′→5′ | 14752 | 14769 |
| 3471 | GAGCAAAGAAGAAGTGTT | NC_045512.2 | 3′→5′ | 14753 | 14770 |
| 3472 | TGAGCAAAGAAGAAGTGT | NC_045512.2 | 3′→5′ | 14754 | 14771 |
| 3473 | CTGAGCAAAGAAGAAGTG | NC_045512.2 | 3′→5′ | 14755 | 14772 |
| 3474 | CCTGAGCAAAGAAGAAGT | NC_045512.2 | 3′→5′ | 14756 | 14773 |
| 3475 | TCCTGAGCAAAGAAGAAG | NC_045512.2 | 3′→5′ | 14757 | 14774 |
| 3476 | ATCCTGAGCAAAGAAGAA | NC_045512.2 | 3′→5′ | 14758 | 14775 |
| 3477 | CATCCTGAGCAAAGAAGA | NC_045512.2 | 3′→5′ | 14759 | 14776 |
| 3478 | CCATCCTGAGCAAAGAAG | NC_045512.2 | 3′→5′ | 14760 | 14777 |
| 3479 | GATATCACACATTGTTGG | NC_045512.2 | 3′→5′ | 14821 | 14838 |
| 3480 | TGATATCACACATTGTTG | NC_045512.2 | 3′→5′ | 14822 | 14839 |
| 3481 | CTGATATCACACATTGTT | NC_045512.2 | 3′→5′ | 14823 | 14840 |
| 3482 | TCTGATATCACACATTGT | NC_045512.2 | 3′→5′ | 14824 | 14841 |
| 3483 | GTCTGATATCACACATTG | NC_045512.2 | 3′→5′ | 14825 | 14842 |
| 3484 | TGTCTGATATCACACATT | NC_045512.2 | 3′→5′ | 14826 | 14843 |
| 3485 | TTGTCTGATATCACACAT | NC_045512.2 | 3′→5′ | 14827 | 14844 |
| 3486 | GTTGTCTGATATCACACA | NC_045512.2 | 3′→5′ | 14828 | 14845 |
| 3487 | AGTTGTCTGATATCACAC | NC_045512.2 | 3′→5′ | 14829 | 14846 |
| 3488 | ATCAACAACTTCAACTAC | NC_045512.2 | 3′→5′ | 14854 | 14871 |
| 3489 | TATCAACAACTTCAACTA | NC_045512.2 | 3′→5′ | 14855 | 14872 |
| 3490 | TTATCAACAACTTCAACT | NC_045512.2 | 3′→5′ | 14856 | 14873 |
| 3491 | ATCGTAACAATCAAAGTA | NC_045512.2 | 3′→5′ | 14875 | 14892 |
| 3492 | CATCGTAACAATCAAAGT | NC_045512.2 | 3′→5′ | 14876 | 14893 |
| 3493 | CCATCGTAACAATCAAAG | NC_045512.2 | 3′→5′ | 14877 | 14894 |
| 3494 | ACCATCGTAACAATCAAA | NC_045512.2 | 3′→5′ | 14878 | 14895 |
| 3495 | CACCATCGTAACAATCAA | NC_045512.2 | 3′→5′ | 14879 | 14896 |
| 3496 | CCACCATCGTAACAATCA | NC_045512.2 | 3′→5′ | 14880 | 14897 |
| 3497 | GCCACCATCGTAACAATC | NC_045512.2 | 3′→5′ | 14881 | 14898 |
| 3498 | AGCCACCATCGTAACAAT | NC_045512.2 | 3′→5′ | 14882 | 14899 |
| 3499 | CAGCCACCATCGTAACAA | NC_045512.2 | 3′→5′ | 14883 | 14900 |
| 3500 | ACAGCCACCATCGTAACA | NC_045512.2 | 3′→5′ | 14884 | 14901 |
| 3501 | TACAGCCACCATCGTAAC | NC_045512.2 | 3′→5′ | 14885 | 14902 |
| 3502 | ATACAGCCACCATCGTAA | NC_045512.2 | 3′→5′ | 14886 | 14903 |
| 3503 | TCTAGCCTTACCCCATTT | NC_045512.2 | 3′→5′ | 14962 | 14979 |
| 3504 | GTCTAGCCTTACCCCATT | NC_045512.2 | 3′→5′ | 14963 | 14980 |
| 3505 | AGTCTAGCCTTACCCCAT | NC_045512.2 | 3′→5′ | 14964 | 14981 |
| 3506 | AAGTCTAGCCTTACCCCA | NC_045512.2 | 3′→5′ | 14965 | 14982 |
| 3507 | AAAGTCTAGCCTTACCCC | NC_045512.2 | 3′→5′ | 14966 | 14983 |
| 3508 | TAAAGTCTAGCCTTACCC | NC_045512.2 | 3′→5′ | 14967 | 14984 |
| 3509 | ATAAAGTCTAGCCTTACC | NC_045512.2 | 3′→5′ | 14968 | 14985 |
| 3510 | AATAAAGTCTAGCCTTAC | NC_045512.2 | 3′→5′ | 14969 | 14986 |
| 3511 | TAATAAAGTCTAGCCTTA | NC_045512.2 | 3′→5′ | 14970 | 14987 |
| 3512 | ATAATAAAGTCTAGCCTT | NC_045512.2 | 3′→5′ | 14971 | 14988 |
| 3513 | CATAATAAAGTCTAGCCT | NC_045512.2 | 3′→5′ | 14972 | 14989 |
| 3514 | TCATAATAAAGTCTAGCC | NC_045512.2 | 3′→5′ | 14973 | 14990 |
| 3515 | ATCCTCATAACTCATTGA | NC_045512.2 | 3′→5′ | 14992 | 15009 |
| 3516 | GATCCTCATAACTCATTG | NC_045512.2 | 3′→5′ | 14993 | 15010 |
| 3517 | TGATCCTCATAACTCATT | NC_045512.2 | 3′→5′ | 14994 | 15011 |
| 3518 | TTGATCCTCATAACTCAT | NC_045512.2 | 3′→5′ | 14995 | 15012 |
| 3519 | CTTGATCCTCATAACTCA | NC_045512.2 | 3′→5′ | 14996 | 15013 |
| 3520 | TCTTGATCCTCATAACTC | NC_045512.2 | 3′→5′ | 14997 | 15014 |
| 3521 | ATCTTGATCCTCATAACT | NC_045512.2 | 3′→5′ | 14998 | 15015 |
| 3522 | CATCTTGATCCTCATAAC | NC_045512.2 | 3′→5′ | 14999 | 15016 |
| 3523 | GCATCTTGATCCTCATAA | NC_045512.2 | 3′→5′ | 15000 | 15017 |
| 3524 | TGCATCTTGATCCTCATA | NC_045512.2 | 3′→5′ | 15001 | 15018 |
| 3525 | GTGCATCTTGATCCTCAT | NC_045512.2 | 3′→5′ | 15002 | 15019 |
| 3526 | AGTGCATCTTGATCCTCA | NC_045512.2 | 3′→5′ | 15003 | 15020 |
| 3527 | AAGATTCATTTGAGTTAT | NC_045512.2 | 3′→5′ | 15055 | 15072 |
| 3528 | TAAGATTCATTTGAGTTA | NC_045512.2 | 3′→5′ | 15056 | 15073 |
| 3529 | TTAAGATTCATTTGAGTT | NC_045512.2 | 3′→5′ | 15057 | 15074 |
| 3530 | CTTAAGATTCATTTGAGT | NC_045512.2 | 3′→5′ | 15058 | 15075 |
| 3531 | ACTTAAGATTCATTTGAG | NC_045512.2 | 3′→5′ | 15059 | 15076 |
| 3532 | TACTTAAGATTCATTTGA | NC_045512.2 | 3′→5′ | 15060 | 15077 |
| 3533 | ATACTTAAGATTCATTTG | NC_045512.2 | 3′→5′ | 15061 | 15078 |
| 3534 | CATACTTAAGATTCATTT | NC_045512.2 | 3′→5′ | 15062 | 15079 |
| 3535 | GCATACTTAAGATTCATT | NC_045512.2 | 3′→5′ | 15063 | 15080 |
| 3536 | GGCATACTTAAGATTCAT | NC_045512.2 | 3′→5′ | 15064 | 15081 |
| 3537 | TGGCATACTTAAGATTCA | NC_045512.2 | 3′→5′ | 15065 | 15082 |
| 3538 | ATGGCATACTTAAGATTC | NC_045512.2 | 3′→5′ | 15066 | 15083 |
| 3539 | AATGGCATACTTAAGATT | NC_045512.2 | 3′→5′ | 15067 | 15084 |
| 3540 | TAATGGCATACTTAAGAT | NC_045512.2 | 3′→5′ | 15068 | 15085 |
| 3541 | CTAATGGCATACTTAAGA | NC_045512.2 | 3′→5′ | 15069 | 15086 |
| 3542 | ACTAATGGCATACTTAAG | NC_045512.2 | 3′→5′ | 15070 | 15087 |
| 3543 | CACTAATGGCATACTTAA | NC_045512.2 | 3′→5′ | 15071 | 15088 |
| 3544 | GCACTAATGGCATACTTA | NC_045512.2 | 3′→5′ | 15072 | 15089 |
| 3545 | TGCACTAATGGCATACTT | NC_045512.2 | 3′→5′ | 15073 | 15090 |
| 3546 | TTGCACTAATGGCATACT | NC_045512.2 | 3′→5′ | 15074 | 15091 |
| 3547 | TTTGCACTAATGGCATAC | NC_045512.2 | 3′→5′ | 15075 | 15092 |
| 3548 | CTTTGCACTAATGGCATA | NC_045512.2 | 3′→5′ | 15076 | 15093 |
| 3549 | TCTTTGCACTAATGGCAT | NC_045512.2 | 3′→5′ | 15077 | 15094 |
| 3550 | TTCTTTGCACTAATGGCA | NC_045512.2 | 3′→5′ | 15078 | 15095 |
| 3551 | ATTCTTTGCACTAATGGC | NC_045512.2 | 3′→5′ | 15079 | 15096 |
| 3552 | TATTCTTTGCACTAATGG | NC_045512.2 | 3′→5′ | 15080 | 15097 |
| 3553 | CTATTCTTTGCACTAATG | NC_045512.2 | 3′→5′ | 15081 | 15098 |
| 3554 | TCTATTCTTTGCACTAAT | NC_045512.2 | 3′→5′ | 15082 | 15099 |
| 3555 | CTCTATTCTTTGCACTAA | NC_045512.2 | 3′→5′ | 15083 | 15100 |
| 3556 | GCTCTATTCTTTGCACTA | NC_045512.2 | 3′→5′ | 15084 | 15101 |
| 3557 | AGCTCTATTCTTTGCACT | NC_045512.2 | 3′→5′ | 15085 | 15102 |
| 3558 | GAGCTCTATTCTTTGCAC | NC_045512.2 | 3′→5′ | 15086 | 15103 |
| 3559 | CGAGCTCTATTCTTTGCA | NC_045512.2 | 3′→5′ | 15087 | 15104 |
| 3560 | GCGAGCTCTATTCTTTGC | NC_045512.2 | 3′→5′ | 15088 | 15105 |
| 3561 | TGCGAGCTCTATTCTTTG | NC_045512.2 | 3′→5′ | 15089 | 15106 |
| 3562 | GTGCGAGCTCTATTCTTT | NC_045512.2 | 3′→5′ | 15090 | 15107 |
| 3563 | GGTGCGAGCTCTATTCTT | NC_045512.2 | 3′→5′ | 15091 | 15108 |
| 3564 | CGGTGCGAGCTCTATTCT | NC_045512.2 | 3′→5′ | 15092 | 15109 |
| 3565 | ACGGTGCGAGCTCTATTC | NC_045512.2 | 3′→5′ | 15093 | 15110 |
| 3566 | TACGGTGCGAGCTCTATT | NC_045512.2 | 3′→5′ | 15094 | 15111 |
| 3567 | CTACGGTGCGAGCTCTAT | NC_045512.2 | 3′→5′ | 15095 | 15112 |
| 3568 | GCTACGGTGCGAGCTCTA | NC_045512.2 | 3′→5′ | 15096 | 15113 |
| 3569 | AGCTACGGTGCGAGCTCT | NC_045512.2 | 3′→5′ | 15097 | 15114 |
| 3570 | CAGCTACGGTGCGAGCTC | NC_045512.2 | 3′→5′ | 15098 | 15115 |
| 3571 | CCAGCTACGGTGCGAGCT | NC_045512.2 | 3′→5′ | 15099 | 15116 |
| 3572 | ACCAGCTACGGTGCGAGC | NC_045512.2 | 3′→5′ | 15100 | 15117 |
| 3573 | CACCAGCTACGGTGCGAG | NC_045512.2 | 3′→5′ | 15101 | 15118 |
| 3574 | ACACCAGCTACGGTGCGA | NC_045512.2 | 3′→5′ | 15102 | 15119 |
| 3575 | GACACCAGCTACGGTGCG | NC_045512.2 | 3′→5′ | 15103 | 15120 |
| 3576 | AGACACCAGCTACGGTGC | NC_045512.2 | 3′→5′ | 15104 | 15121 |
| 3577 | GAGACACCAGCTACGGTG | NC_045512.2 | 3′→5′ | 15105 | 15122 |
| 3578 | AGAGACACCAGCTACGGT | NC_045512.2 | 3′→5′ | 15106 | 15123 |
| 3579 | TAGAGACACCAGCTACGG | NC_045512.2 | 3′→5′ | 15107 | 15124 |
| 3580 | ATAGAGACACCAGCTACG | NC_045512.2 | 3′→5′ | 15108 | 15125 |
| 3581 | GATAGAGACACCAGCTAC | NC_045512.2 | 3′→5′ | 15109 | 15126 |
| 3582 | AGATAGAGACACCAGCTA | NC_045512.2 | 3′→5′ | 15110 | 15127 |
| 3583 | CAGATAGAGACACCAGCT | NC_045512.2 | 3′→5′ | 15111 | 15128 |
| 3584 | ACAGATAGAGACACCAGC | NC_045512.2 | 3′→5′ | 15112 | 15129 |
| 3585 | TACAGATAGAGACACCAG | NC_045512.2 | 3′→5′ | 15113 | 15130 |
| 3586 | CTACAGATAGAGACACCA | NC_045512.2 | 3′→5′ | 15114 | 15131 |
| 3587 | ACTACAGATAGAGACACC | NC_045512.2 | 3′→5′ | 15115 | 15132 |
| 3588 | TACTACAGATAGAGACAC | NC_045512.2 | 3′→5′ | 15116 | 15133 |
| 3589 | GTACTACAGATAGAGACA | NC_045512.2 | 3′→5′ | 15117 | 15134 |
| 3590 | AGTACTACAGATAGAGAC | NC_045512.2 | 3′→5′ | 15118 | 15135 |
| 3591 | TAGTACTACAGATAGAGA | NC_045512.2 | 3′→5′ | 15119 | 15136 |
| 3592 | ATAGTACTACAGATAGAG | NC_045512.2 | 3′→5′ | 15120 | 15137 |
| 3593 | CATAGTACTACAGATAGA | NC_045512.2 | 3′→5′ | 15121 | 15138 |
| 3594 | TCATAGTACTACAGATAG | NC_045512.2 | 3′→5′ | 15122 | 15139 |
| 3595 | GTCATAGTACTACAGATA | NC_045512.2 | 3′→5′ | 15123 | 15140 |
| 3596 | TCTAGTGGCGGCTATTGA | NC_045512.2 | 3′→5′ | 15172 | 15189 |
| 3597 | CTCTAGTGGCGGCTATTG | NC_045512.2 | 3′→5′ | 15173 | 15190 |
| 3598 | CCTCTAGTGGCGGCTATT | NC_045512.2 | 3′→5′ | 15174 | 15191 |
| 3599 | TCCTCTAGTGGCGGCTAT | NC_045512.2 | 3′→5′ | 15175 | 15192 |
| 3600 | CTCCTCTAGTGGCGGCTA | NC_045512.2 | 3′→5′ | 15176 | 15193 |
| 3601 | GCTCCTCTAGTGGCGGCT | NC_045512.2 | 3′→5′ | 15177 | 15194 |
| 3602 | AGCTCCTCTAGTGGCGGC | NC_045512.2 | 3′→5′ | 15178 | 15195 |
| 3603 | TAGCTCCTCTAGTGGCGG | NC_045512.2 | 3′→5′ | 15179 | 15196 |
| 3604 | GTAGCTCCTCTAGTGGCG | NC_045512.2 | 3′→5′ | 15180 | 15197 |
| 3605 | AGTAGCTCCTCTAGTGGC | NC_045512.2 | 3′→5′ | 15181 | 15198 |
| 3606 | CAGTAGCTCCTCTAGTGG | NC_045512.2 | 3′→5′ | 15182 | 15199 |
| 3607 | ACAGTAGCTCCTCTAGTG | NC_045512.2 | 3′→5′ | 15183 | 15200 |
| 3608 | CATGTTAGGCATGGCTCT | NC_045512.2 | 3′→5′ | 15310 | 15327 |
| 3609 | GCATGTTAGGCATGGCTC | NC_045512.2 | 3′→5′ | 15311 | 15328 |
| 3610 | AGCATGTTAGGCATGGCT | NC_045512.2 | 3′→5′ | 15312 | 15329 |
| 3611 | AAGCATGTTAGGCATGGC | NC_045512.2 | 3′→5′ | 15313 | 15330 |
| 3612 | TAAGCATGTTAGGCATGG | NC_045512.2 | 3′→5′ | 15314 | 15331 |
| 3613 | CTAAGCATGTTAGGCATG | NC_045512.2 | 3′→5′ | 15315 | 15332 |
| 3614 | TTTGCGAGCAAGAACAAG | NC_045512.2 | 3′→5′ | 15346 | 15363 |
| 3615 | GTTTGCGAGCAAGAACAA | NC_045512.2 | 3′→5′ | 15347 | 15364 |
| 3616 | TGTTTGCGAGCAAGAACA | NC_045512.2 | 3′→5′ | 15348 | 15365 |
| 3617 | ATGTTTGCGAGCAAGAAC | NC_045512.2 | 3′→5′ | 15349 | 15366 |
| 3618 | TATGTTTGCGAGCAAGAA | NC_045512.2 | 3′→5′ | 15350 | 15367 |
| 3619 | ATTAGCATAAGCAGTTGT | NC_045512.2 | 3′→5′ | 15496 | 15513 |
| 3620 | TATTAGCATAAGCAGTTG | NC_045512.2 | 3′→5′ | 15497 | 15514 |
| 3621 | CTATTAGCATAAGCAGTT | NC_045512.2 | 3′→5′ | 15498 | 15515 |
| 3622 | ACTATTAGCATAAGCAGT | NC_045512.2 | 3′→5′ | 15499 | 15516 |
| 3623 | CACTATTAGCATAAGCAG | NC_045512.2 | 3′→5′ | 15500 | 15517 |
| 3624 | ACACTATTAGCATAAGCA | NC_045512.2 | 3′→5′ | 15501 | 15518 |
| 3625 | TCTATAGAGACACTCATA | NC_045512.2 | 3′→5′ | 15622 | 15639 |
| 3626 | TTCTATAGAGACACTCAT | NC_045512.2 | 3′→5′ | 15623 | 15640 |
| 3627 | TTTCTATAGAGACACTCA | NC_045512.2 | 3′→5′ | 15624 | 15641 |
| 3628 | ATTTCTATAGAGACACTC | NC_045512.2 | 3′→5′ | 15625 | 15642 |
| 3629 | TATTTCTATAGAGACACT | NC_045512.2 | 3′→5′ | 15626 | 15643 |
| 3630 | CTATTTCTATAGAGACAC | NC_045512.2 | 3′→5′ | 15627 | 15644 |
| 3631 | AAGGTCAGTCTCAGTCCA | NC_045512.2 | 3′→5′ | 15838 | 15855 |
| 3632 | TAAGGTCAGTCTCAGTCC | NC_045512.2 | 3′→5′ | 15839 | 15856 |
| 3633 | GTAAGGTCAGTCTCAGTC | NC_045512.2 | 3′→5′ | 15840 | 15857 |
| 3634 | AGTAAGGTCAGTCTCAGT | NC_045512.2 | 3′→5′ | 15841 | 15858 |
| 3635 | TAGTAAGGTCAGTCTCAG | NC_045512.2 | 3′→5′ | 15842 | 15859 |
| 3636 | TTAGTAAGGTCAGTCTCA | NC_045512.2 | 3′→5′ | 15843 | 15860 |
| 3637 | TTTAGTAAGGTCAGTCTC | NC_045512.2 | 3′→5′ | 15844 | 15861 |
| 3638 | CTTTAGTAAGGTCAGTCT | NC_045512.2 | 3′→5′ | 15845 | 15862 |
| 3639 | CCTTTAGTAAGGTCAGTC | NC_045512.2 | 3′→5′ | 15846 | 15863 |
| 3640 | TCCTTTAGTAAGGTCAGT | NC_045512.2 | 3′→5′ | 15847 | 15864 |
| 3641 | GTCCTTTAGTAAGGTCAG | NC_045512.2 | 3′→5′ | 15848 | 15865 |
| 3642 | GGTCCTTTAGTAAGGTCA | NC_045512.2 | 3′→5′ | 15849 | 15866 |
| 3643 | AGGTCCTTTAGTAAGGTC | NC_045512.2 | 3′→5′ | 15850 | 15867 |
| 3644 | GAGGTCCTTTAGTAAGGT | NC_045512.2 | 3′→5′ | 15851 | 15868 |
| 3645 | TGAGGTCCTTTAGTAAGG | NC_045512.2 | 3′→5′ | 15852 | 15869 |
| 3646 | TTTAACTAGCATTGTATG | NC_045512.2 | 3′→5′ | 15886 | 15903 |
| 3647 | GTTTAACTAGCATTGTAT | NC_045512.2 | 3′→5′ | 15887 | 15904 |
| 3648 | TGTTTAACTAGCATTGTA | NC_045512.2 | 3′→5′ | 15888 | 15905 |
| 3649 | AAGTGTACCATCTGTTTT | NC_045512.2 | 3′→5′ | 15985 | 16002 |
| 3650 | TAAGTGTACCATCTGTTT | NC_045512.2 | 3′→5′ | 15986 | 16003 |
| 3651 | ATAAGTGTACCATCTGTT | NC_045512.2 | 3′→5′ | 15987 | 16004 |
| 3652 | CATAAGTGTACCATCTGT | NC_045512.2 | 3′→5′ | 15988 | 16005 |
| 3653 | TCATAAGTGTACCATCTG | NC_045512.2 | 3′→5′ | 15989 | 16006 |
| 3654 | ATCATAAGTGTACCATCT | NC_045512.2 | 3′→5′ | 15990 | 16007 |
| 3655 | AATCATAAGTGTACCATC | NC_045512.2 | 3′→5′ | 15991 | 16008 |
| 3656 | CAATCATAAGTGTACCAT | NC_045512.2 | 3′→5′ | 15992 | 16009 |
| 3657 | TCAATCATAAGTGTACCA | NC_045512.2 | 3′→5′ | 15993 | 16010 |
| 3658 | AGCATACTCCTGATTAGG | NC_045512.2 | 3′→5′ | 16057 | 16074 |
| 3659 | CAGCATACTCCTGATTAG | NC_045512.2 | 3′→5′ | 16058 | 16075 |
| 3660 | TCAGCATACTCCTGATTA | NC_045512.2 | 3′→5′ | 16059 | 16076 |
| 3661 | ATCAGCATACTCCTGATT | NC_045512.2 | 3′→5′ | 16060 | 16077 |
| 3662 | CATCAGCATACTCCTGAT | NC_045512.2 | 3′→5′ | 16061 | 16078 |
| 3663 | ACATCAGCATACTCCTGA | NC_045512.2 | 3′→5′ | 16062 | 16079 |
| 3664 | ATAAAACTCAGGTTCCCA | NC_045512.2 | 3′→5′ | 16186 | 16203 |
| 3665 | CATAAAACTCAGGTTCCC | NC_045512.2 | 3′→5′ | 16187 | 16204 |
| 3666 | TCATAAAACTCAGGTTCC | NC_045512.2 | 3′→5′ | 16188 | 16205 |
| 3667 | AATAGCTCATACCTCCTA | NC_045512.2 | 3′→5′ | 16430 | 16447 |
| 3668 | TAATAGCTCATACCTCCT | NC_045512.2 | 3′→5′ | 16431 | 16448 |
| 3669 | TTCAAAGGTGTACTCTCC | NC_045512.2 | 3′→5′ | 16822 | 16839 |
| 3670 | TTTCAAAGGTGTACTCTC | NC_045512.2 | 3′→5′ | 16823 | 16840 |
| 3671 | TTTTCAAAGGTGTACTCT | NC_045512.2 | 3′→5′ | 16824 | 16841 |
| 3672 | TTTTTCAAAGGTGTACTC | NC_045512.2 | 3′→5′ | 16825 | 16842 |
| 3673 | CTTTTTCAAAGGTGTACT | NC_045512.2 | 3′→5′ | 16826 | 16843 |
| 3674 | CCTTTTTCAAAGGTGTAC | NC_045512.2 | 3′→5′ | 16827 | 16844 |
| 3675 | ACCTTTTTCAAAGGTGTA | NC_045512.2 | 3′→5′ | 16828 | 16845 |
| 3676 | CACCTTTTTCAAAGGTGT | NC_045512.2 | 3′→5′ | 16829 | 16846 |
| 3677 | TCACCTTTTTCAAAGGTG | NC_045512.2 | 3′→5′ | 16830 | 16847 |
| 3678 | GTCACCTTTTTCAAAGGT | NC_045512.2 | 3′→5′ | 16831 | 16848 |
| 3679 | AGTCACCTTTTTCAAAGG | NC_045512.2 | 3′→5′ | 16832 | 16849 |
| 3680 | TAGTCACCTTTTTCAAAG | NC_045512.2 | 3′→5′ | 16833 | 16850 |
| 3681 | ATAGTCACCTTTTTCAAA | NC_045512.2 | 3′→5′ | 16834 | 16851 |
| 3682 | CATAGTCACCTTTTTCAA | NC_045512.2 | 3′→5′ | 16835 | 16852 |
| 3683 | CCATAGTCACCTTTTTCA | NC_045512.2 | 3′→5′ | 16836 | 16853 |
| 3684 | ACCATAGTCACCTTTTTC | NC_045512.2 | 3′→5′ | 16837 | 16854 |
| 3685 | CACCATAGTCACCTTTTT | NC_045512.2 | 3′→5′ | 16838 | 16855 |
| 3686 | TCACCATAGTCACCTTTT | NC_045512.2 | 3′→5′ | 16839 | 16856 |
| 3687 | ATCACCATAGTCACCTTT | NC_045512.2 | 3′→5′ | 16840 | 16857 |
| 3688 | CATCACCATAGTCACCTT | NC_045512.2 | 3′→5′ | 16841 | 16858 |
| 3689 | GCATCACCATAGTCACCT | NC_045512.2 | 3′→5′ | 16842 | 16859 |
| 3690 | AGCATCACCATAGTCACC | NC_045512.2 | 3′→5′ | 16843 | 16860 |
| 3691 | CAGCATCACCATAGTCAC | NC_045512.2 | 3′→5′ | 16844 | 16861 |
| 3692 | ACAGCATCACCATAGTCA | NC_045512.2 | 3′→5′ | 16845 | 16862 |
| 3693 | AACAGCATCACCATAGTC | NC_045512.2 | 3′→5′ | 16846 | 16863 |
| 3694 | CAACAGCATCACCATAGT | NC_045512.2 | 3′→5′ | 16847 | 16864 |
| 3695 | ACAACAGCATCACCATAG | NC_045512.2 | 3′→5′ | 16848 | 16865 |
| 3696 | GTGCTCTTGTGGCACTAG | NC_045512.2 | 3′→5′ | 16954 | 16971 |
| 3697 | AGTGCTCTTGTGGCACTA | NC_045512.2 | 3′→5′ | 16955 | 16972 |
| 3698 | TAGTGCTCTTGTGGCACT | NC_045512.2 | 3′→5′ | 16956 | 16973 |
| 3699 | ATAGTGCTCTTGTGGCAC | NC_045512.2 | 3′→5′ | 16957 | 16974 |
| 3700 | CATAGTGCTCTTGTGGCA | NC_045512.2 | 3′→5′ | 16958 | 16975 |
| 3701 | ACATAGTGCTCTTGTGGC | NC_045512.2 | 3′→5′ | 16959 | 16976 |
| 3702 | AGAAAACTCATCTGAGAT | NC_045512.2 | 3′→5′ | 17008 | 17025 |
| 3703 | TAGAAAACTCATCTGAGA | NC_045512.2 | 3′→5′ | 17009 | 17026 |
| 3704 | CTAGAAAACTCATCTGAG | NC_045512.2 | 3′→5′ | 17010 | 17027 |
| 3705 | GCTAGAAAACTCATCTGA | NC_045512.2 | 3′→5′ | 17011 | 17028 |
| 3706 | TGCTAGAAAACTCATCTG | NC_045512.2 | 3′→5′ | 17012 | 17029 |
| 3707 | TTGCTAGAAAACTCATCT | NC_045512.2 | 3′→5′ | 17013 | 17030 |
| 3708 | ATTGCTAGAAAACTCATC | NC_045512.2 | 3′→5′ | 17014 | 17031 |
| 3709 | CATTGCTAGAAAACTCAT | NC_045512.2 | 3′→5′ | 17015 | 17032 |
| 3710 | ACATTGCTAGAAAACTCA | NC_045512.2 | 3′→5′ | 17016 | 17033 |
| 3711 | AACATTGCTAGAAAACTC | NC_045512.2 | 3′→5′ | 17017 | 17034 |
| 3712 | CAACATTGCTAGAAAACT | NC_045512.2 | 3′→5′ | 17018 | 17035 |
| 3713 | GCAACATTGCTAGAAAAC | NC_045512.2 | 3′→5′ | 17019 | 17036 |
| 3714 | TGCAACATTGCTAGAAAA | NC_045512.2 | 3′→5′ | 17020 | 17037 |
| 3715 | TTGCAACATTGCTAGAAA | NC_045512.2 | 3′→5′ | 17021 | 17038 |
| 3716 | TTTGCAACATTGCTAGAA | NC_045512.2 | 3′→5′ | 17022 | 17039 |
| 3717 | ATTTGCAACATTGCTAGA | NC_045512.2 | 3′→5′ | 17023 | 17040 |
| 3718 | AATTTGCAACATTGCTAG | NC_045512.2 | 3′→5′ | 17024 | 17041 |
| 3719 | TAATTTGCAACATTGCTA | NC_045512.2 | 3′→5′ | 17025 | 17042 |
| 3720 | ACCAGTACCAGGTGGTCC | NC_045512.2 | 3′→5′ | 17080 | 17097 |
| 3721 | TACCAGTACCAGGTGGTC | NC_045512.2 | 3′→5′ | 17081 | 17098 |
| 3722 | TTACCAGTACCAGGTGGT | NC_045512.2 | 3′→5′ | 17082 | 17099 |
| 3723 | CTTACCAGTACCAGGTGG | NC_045512.2 | 3′→5′ | 17083 | 17100 |
| 3724 | TCTTACCAGTACCAGGTG | NC_045512.2 | 3′→5′ | 17084 | 17101 |
| 3725 | CTCTTACCAGTACCAGGT | NC_045512.2 | 3′→5′ | 17085 | 17102 |
| 3726 | ACTCTTACCAGTACCAGG | NC_045512.2 | 3′→5′ | 17086 | 17103 |
| 3727 | GACTCTTACCAGTACCAG | NC_045512.2 | 3′→5′ | 17087 | 17104 |
| 3728 | TGACTCTTACCAGTACCA | NC_045512.2 | 3′→5′ | 17088 | 17105 |
| 3729 | ATGACTCTTACCAGTACC | NC_045512.2 | 3′→5′ | 17089 | 17106 |
| 3730 | AATGACTCTTACCAGTAC | NC_045512.2 | 3′→5′ | 17090 | 17107 |
| 3731 | AAATGACTCTTACCAGTA | NC_045512.2 | 3′→5′ | 17091 | 17108 |
| 3732 | AAAATGACTCTTACCAGT | NC_045512.2 | 3′→5′ | 17092 | 17109 |
| 3733 | CAAAATGACTCTTACCAG | NC_045512.2 | 3′→5′ | 17093 | 17110 |
| 3734 | GCAAAATGACTCTTACCA | NC_045512.2 | 3′→5′ | 17094 | 17111 |
| 3735 | ATACACTATGCGAGCAGA | NC_045512.2 | 3′→5′ | 17137 | 17154 |
| 3736 | TATACACTATGCGAGCAG | NC_045512.2 | 3′→5′ | 17138 | 17155 |
| 3737 | GTATACACTATGCGAGCA | NC_045512.2 | 3′→5′ | 17139 | 17156 |
| 3738 | TGAATTCACTTTGAATTT | NC_045512.2 | 3′→5′ | 17269 | 17286 |
| 3739 | TTGAATTCACTTTGAATT | NC_045512.2 | 3′→5′ | 17270 | 17287 |
| 3740 | GTTGAATTCACTTTGAAT | NC_045512.2 | 3′→5′ | 17271 | 17288 |
| 3741 | TGTTGAATTCACTTTGAA | NC_045512.2 | 3′→5′ | 17272 | 17289 |
| 3742 | GAACATGTCTGGACCTAT | NC_045512.2 | 3′→5′ | 17530 | 17547 |
| 3743 | GGAACATGTCTGGACCTA | NC_045512.2 | 3′→5′ | 17531 | 17548 |
| 3744 | AGGAACATGTCTGGACCT | NC_045512.2 | 3′→5′ | 17532 | 17549 |
| 3745 | AATTTCAGCAGGACAACG | NC_045512.2 | 3′→5′ | 17563 | 17580 |
| 3746 | CAATTTCAGCAGGACAAC | NC_045512.2 | 3′→5′ | 17564 | 17581 |
| 3747 | ACAATTTCAGCAGGACAA | NC_045512.2 | 3′→5′ | 17565 | 17582 |
| 3748 | TGCAGATGAAACATCATG | NC_045512.2 | 3′→5′ | 17680 | 17697 |
| 3749 | TTGCAGATGAAACATCAT | NC_045512.2 | 3′→5′ | 17681 | 17698 |
| 3750 | ATTGCAGATGAAACATCA | NC_045512.2 | 3′→5′ | 17682 | 17699 |
| 3751 | AGCTTTTCTCCAAGCAGG | NC_045512.2 | 3′→5′ | 17746 | 17763 |
| 3752 | CAGCTTTTCTCCAAGCAG | NC_045512.2 | 3′→5′ | 17747 | 17764 |
| 3753 | ACAGCTTTTCTCCAAGCA | NC_045512.2 | 3′→5′ | 17748 | 17765 |
| 3754 | GAATATGACATAGTCATA | NC_045512.2 | 3′→5′ | 17857 | 17874 |
| 3755 | TGAATATGACATAGTCAT | NC_045512.2 | 3′→5′ | 17858 | 17875 |
| 3756 | GTGAATATGACATAGTCA | NC_045512.2 | 3′→5′ | 17859 | 17876 |
| 3757 | TCTATCAGACATTATGCA | NC_045512.2 | 3′→5′ | 17956 | 17973 |
| 3758 | CTCTATCAGACATTATGC | NC_045512.2 | 3′→5′ | 17957 | 17974 |
| 3759 | TCTCTATCAGACATTATG | NC_045512.2 | 3′→5′ | 17958 | 17975 |
| 3760 | GTGTGTAGGTGCCTGTGT | NC_045512.2 | 3′→5′ | 18100 | 18117 |
| 3761 | GGTGTGTAGGTGCCTGTG | NC_045512.2 | 3′→5′ | 18101 | 18118 |
| 3762 | AGGTGTGTAGGTGCCTGT | NC_045512.2 | 3′→5′ | 18102 | 18119 |
| 3763 | GAGGTGTGTAGGTGCCTG | NC_045512.2 | 3′→5′ | 18103 | 18120 |
| 3764 | TGAGGTGTGTAGGTGCCT | NC_045512.2 | 3′→5′ | 18104 | 18121 |
| 3765 | CTGAGGTGTGTAGGTGCC | NC_045512.2 | 3′→5′ | 18105 | 18122 |
| 3766 | CATCATAGAGATGAGTCT | NC_045512.2 | 3′→5′ | 18196 | 18213 |
| 3767 | CCATCATAGAGATGAGTC | NC_045512.2 | 3′→5′ | 18197 | 18214 |
| 3768 | CCCATCATAGAGATGAGT | NC_045512.2 | 3′→5′ | 18198 | 18215 |
| 3769 | ACCCATCATAGAGATGAG | NC_045512.2 | 3′→5′ | 18199 | 18216 |
| 3770 | AACCCATCATAGAGATGA | NC_045512.2 | 3′→5′ | 18200 | 18217 |
| 3771 | AAACCCATCATAGAGATG | NC_045512.2 | 3′→5′ | 18201 | 18218 |
| 3772 | CACATTTTCTAAACTCTG | NC_045512.2 | 3′→5′ | 19618 | 19635 |
| 3773 | CCACATTTTCTAAACTCT | NC_045512.2 | 3′→5′ | 19619 | 19636 |
| 3774 | GCCACATTTTCTAAACTC | NC_045512.2 | 3′→5′ | 19620 | 19637 |
| 3775 | AGCCACATTTTCTAAACT | NC_045512.2 | 3′→5′ | 19621 | 19638 |
| 3776 | AAGCCACATTTTCTAAAC | NC_045512.2 | 3′→5′ | 19622 | 19639 |
| 3777 | CTTAGCCCAAAGCTCAAA | NC_045512.2 | 3′→5′ | 19783 | 19800 |
| 3778 | GCTTAGCCCAAAGCTCAA | NC_045512.2 | 3′→5′ | 19784 | 19801 |
| 3779 | CGCTTAGCCCAAAGCTCA | NC_045512.2 | 3′→5′ | 19785 | 19802 |
| 3780 | ACCCAAATTATTGAGTAT | NC_045512.2 | 3′→5′ | 19831 | 19848 |
| 3781 | CACCCAAATTATTGAGTA | NC_045512.2 | 3′→5′ | 19832 | 19849 |
| 3782 | ACACCCAAATTATTGAGT | NC_045512.2 | 3′→5′ | 19833 | 19850 |
| 3783 | AATTAATGTGACTCCATT | NC_045512.2 | 3′→5′ | 20107 | 20124 |
| 3784 | CAATTAATGTGACTCCAT | NC_045512.2 | 3′→5′ | 20108 | 20125 |
| 3785 | CCAATTAATGTGACTCCA | NC_045512.2 | 3′→5′ | 20109 | 20126 |
| 3786 | TCCAATTAATGTGACTCC | NC_045512.2 | 3′→5′ | 20110 | 20127 |
| 3787 | CTCCAATTAATGTGACTC | NC_045512.2 | 3′→5′ | 20111 | 20128 |
| 3788 | TCTCCAATTAATGTGACT | NC_045512.2 | 3′→5′ | 20112 | 20129 |
| 3789 | TTCTCCAATTAATGTGAC | NC_045512.2 | 3′→5′ | 20113 | 20130 |
| 3790 | TGCGACATTCATCATTAT | NC_045512.2 | 3′→5′ | 20776 | 20793 |
| 3791 | TTGCGACATTCATCATTA | NC_045512.2 | 3′→5′ | 20777 | 20794 |
| 3792 | TTTGCGACATTCATCATT | NC_045512.2 | 3′→5′ | 20778 | 20795 |
| 3793 | TCTGTTGTTTTCTCTAAT | NC_045512.2 | 3′→5′ | 21502 | 21519 |
| 3794 | CTCTGTTGTTTTCTCTAA | NC_045512.2 | 3′→5′ | 21503 | 21520 |
| 3795 | ACTCTGTTGTTTTCTCTA | NC_045512.2 | 3′→5′ | 21504 | 21521 |
| 3796 | AACTCTGTTGTTTTCTCT | NC_045512.2 | 3′→5′ | 21505 | 21522 |
| 3797 | CAACTCTGTTGTTTTCTC | NC_045512.2 | 3′→5′ | 21506 | 21523 |
| 3798 | ACAACTCTGTTGTTTTCT | NC_045512.2 | 3′→5′ | 21507 | 21524 |
| 3799 | GTTCTCATAGAGAACATT | NC_045512.2 | 3′→5′ | 24302 | 24319 |
| 3800 | GGTTCTCATAGAGAACAT | NC_045512.2 | 3′→5′ | 24303 | 24320 |
| 3801 | TGGTTCTCATAGAGAACA | NC_045512.2 | 3′→5′ | 24304 | 24321 |
| 3802 | TTGGTTCTCATAGAGAAC | NC_045512.2 | 3′→5′ | 24305 | 24322 |
| 3803 | TTTGGTTCTCATAGAGAA | NC_045512.2 | 3′→5′ | 24306 | 24323 |
| 3804 | TTTTGGTTCTCATAGAGA | NC_045512.2 | 3′→5′ | 24307 | 24324 |
| 3805 | TTTTTGGTTCTCATAGAG | NC_045512.2 | 3′→5′ | 24308 | 24325 |
| 3806 | GCTAAGTTGTTTAACAAG | NC_045512.2 | 3′→5′ | 24446 | 24463 |
| 3807 | AGCTAAGTTGTTTAACAA | NC_045512.2 | 3′→5′ | 24447 | 24464 |
| 3808 | GAGCTAAGTTGTTTAACA | NC_045512.2 | 3′→5′ | 24448 | 24465 |
| 3809 | AGCAAGATTAGCAGAAGC | NC_045512.2 | 3′→5′ | 24620 | 24637 |
| 3810 | CAGCAAGATTAGCAGAAG | NC_045512.2 | 3′→5′ | 24621 | 24638 |
| 3811 | GCAGCAAGATTAGCAGAA | NC_045512.2 | 3′→5′ | 24622 | 24639 |
| 3812 | AGCAGCAAGATTAGCAGA | NC_045512.2 | 3′→5′ | 24623 | 24640 |
| 3813 | TAGCAGCAAGATTAGCAG | NC_045512.2 | 3′→5′ | 24624 | 24641 |
| 3814 | GTAGCAGCAAGATTAGCA | NC_045512.2 | 3′→5′ | 24625 | 24642 |
| 3815 | AGTAGCAGCAAGATTAGC | NC_045512.2 | 3′→5′ | 24626 | 24643 |
| 3816 | TAGTAGCAGCAAGATTAG | NC_045512.2 | 3′→5′ | 24627 | 24644 |
| 3817 | TTAGTAGCAGCAAGATTA | NC_045512.2 | 3′→5′ | 24628 | 24645 |
| 3818 | TTTAGTAGCAGCAAGATT | NC_045512.2 | 3′→5′ | 24629 | 24646 |
| 3819 | TTTTAGTAGCAGCAAGAT | NC_045512.2 | 3′→5′ | 24630 | 24647 |
| 3820 | ATTTTAGTAGCAGCAAGA | NC_045512.2 | 3′→5′ | 24631 | 24648 |
| 3821 | CATTTTAGTAGCAGCAAG | NC_045512.2 | 3′→5′ | 24632 | 24649 |
| 3822 | ACATTTTAGTAGCAGCAA | NC_045512.2 | 3′→5′ | 24633 | 24650 |
| 3823 | GACATTTTAGTAGCAGCA | NC_045512.2 | 3′→5′ | 24634 | 24651 |
| 3824 | TCTTTTTGATTGTCCAAG | NC_045512.2 | 3′→5′ | 24662 | 24679 |
| 3825 | CTCTTTTTGATTGTCCAA | NC_045512.2 | 3′→5′ | 24663 | 24680 |
| 3826 | ACTCTTTTTGATTGTCCA | NC_045512.2 | 3′→5′ | 24664 | 24681 |
| 3827 | AACTCTTTTTGATTGTCC | NC_045512.2 | 3′→5′ | 24665 | 24682 |
| 3828 | CAACTCTTTTTGATTGTC | NC_045512.2 | 3′→5′ | 24666 | 24683 |
| 3829 | TCAACTCTTTTTGATTGT | NC_045512.2 | 3′→5′ | 24667 | 24684 |
| 3830 | ATCTGGTGATGTATGATT | NC_045512.2 | 3′→5′ | 25034 | 25051 |
| 3831 | CATCTGGTGATGTATGAT | NC_045512.2 | 3′→5′ | 25035 | 25052 |
| 3832 | ACATCTGGTGATGTATGA | NC_045512.2 | 3′→5′ | 25036 | 25053 |
| 3833 | AACATCTGGTGATGTATG | NC_045512.2 | 3′→5′ | 25037 | 25054 |
| 3834 | CAACATCTGGTGATGTAT | NC_045512.2 | 3′→5′ | 25038 | 25055 |
| 3835 | TCAACATCTGGTGATGTA | NC_045512.2 | 3′→5′ | 25039 | 25056 |
| 3836 | ATCAACATCTGGTGATGT | NC_045512.2 | 3′→5′ | 25040 | 25057 |
| 3837 | TGAGGCGGTCAATTTCTT | NC_045512.2 | 3′→5′ | 25104 | 25121 |
| 3838 | TTGAGGCGGTCAATTTCT | NC_045512.2 | 3′→5′ | 25105 | 25122 |
| 3839 | ATTGAGGCGGTCAATTTC | NC_045512.2 | 3′→5′ | 25106 | 25123 |
| 3840 | CATTGAGGCGGTCAATTT | NC_045512.2 | 3′→5′ | 25107 | 25124 |
| 3841 | TCATTGAGGCGGTCAATT | NC_045512.2 | 3′→5′ | 25108 | 25125 |
| 3842 | CTCATTGAGGCGGTCAAT | NC_045512.2 | 3′→5′ | 25109 | 25126 |
| 3843 | CCTCATTGAGGCGGTCAA | NC_045512.2 | 3′→5′ | 25110 | 25127 |
| 3844 | ACCTCATTGAGGCGGTCA | NC_045512.2 | 3′→5′ | 25111 | 25128 |
| 3845 | TGTGTAATGTAATTTGAC | NC_045512.2 | 3′→5′ | 25364 | 25381 |
| 3846 | ATGTGTAATGTAATTTGA | NC_045512.2 | 3′→5′ | 25365 | 25382 |
| 3847 | TATGTGTAATGTAATTTG | NC_045512.2 | 3′→5′ | 25366 | 25383 |
| 3848 | TTATGTGTAATGTAATTT | NC_045512.2 | 3′→5′ | 25367 | 25384 |
| 3849 | TTTATGTGTAATGTAATT | NC_045512.2 | 3′→5′ | 25368 | 25385 |
| 3850 | GTTTATGTGTAATGTAAT | NC_045512.2 | 3′→5′ | 25369 | 25386 |
| 3851 | CGTTTATGTGTAATGTAA | NC_045512.2 | 3′→5′ | 25370 | 25387 |
| 3852 | AAGGGAGTGAGGCTTGTA | NC_045512.2 | 3′→5′ | 25502 | 25519 |
| 3853 | AAAGGGAGTGAGGCTTGT | NC_045512.2 | 3′→5′ | 25503 | 25520 |
| 3854 | GAAAGGGAGTGAGGCTTG | NC_045512.2 | 3′→5′ | 25504 | 25521 |
| 3855 | CGAAAGGGAGTGAGGCTT | NC_045512.2 | 3′→5′ | 25505 | 25522 |
| 3856 | CCGAAAGGGAGTGAGGCT | NC_045512.2 | 3′→5′ | 25506 | 25523 |
| 3857 | TCCGAAAGGGAGTGAGGC | NC_045512.2 | 3′→5′ | 25507 | 25524 |
| 3858 | ATCCGAAAGGGAGTGAGG | NC_045512.2 | 3′→5′ | 25508 | 25525 |
| 3859 | CATCCGAAAGGGAGTGAG | NC_045512.2 | 3′→5′ | 25509 | 25526 |
| 3860 | CCATCCGAAAGGGAGTGA | NC_045512.2 | 3′→5′ | 25510 | 25527 |
| 3861 | GCCATCCGAAAGGGAGTG | NC_045512.2 | 3′→5′ | 25511 | 25528 |
| 3862 | AGCCATCCGAAAGGGAGT | NC_045512.2 | 3′→5′ | 25512 | 25529 |
| 3863 | AAGCCATCCGAAAGGGAG | NC_045512.2 | 3′→5′ | 25513 | 25530 |
| 3864 | GCTAGTAGTCGTCGTCGG | NC_045512.2 | 3′→5′ | 26191 | 26208 |
| 3865 | CGCTAGTAGTCGTCGTCG | NC_045512.2 | 3′→5′ | 26192 | 26209 |
| 3866 | ACGCTAGTAGTCGTCGTC | NC_045512.2 | 3′→5′ | 26193 | 26210 |
| 3867 | CACGCTAGTAGTCGTCGT | NC_045512.2 | 3′→5′ | 26194 | 26211 |
| 3868 | GCACGCTAGTAGTCGTCG | NC_045512.2 | 3′→5′ | 26195 | 26212 |
| 3869 | GGCACGCTAGTAGTCGTC | NC_045512.2 | 3′→5′ | 26196 | 26213 |
| 3870 | AGGCACGCTAGTAGTCGT | NC_045512.2 | 3′→5′ | 26197 | 26214 |
| 3871 | AAGGCACGCTAGTAGTCG | NC_045512.2 | 3′→5′ | 26198 | 26215 |
| 3872 | AAAGGCACGCTAGTAGTC | NC_045512.2 | 3′→5′ | 26199 | 26216 |
| 3873 | CAAAGGCACGCTAGTAGT | NC_045512.2 | 3′→5′ | 26200 | 26217 |
| 3874 | ACAAAGGCACGCTAGTAG | NC_045512.2 | 3′→5′ | 26201 | 26218 |
| 3875 | TACAAAGGCACGCTAGTA | NC_045512.2 | 3′→5′ | 26202 | 26219 |
| 3876 | TTACAAAGGCACGCTAGT | NC_045512.2 | 3′→5′ | 26203 | 26220 |
| 3877 | CTTACAAAGGCACGCTAG | NC_045512.2 | 3′→5′ | 26204 | 26221 |
| 3878 | GCTTACAAAGGCACGCTA | NC_045512.2 | 3′→5′ | 26205 | 26222 |
| 3879 | TGCTTACAAAGGCACGCT | NC_045512.2 | 3′→5′ | 26206 | 26223 |
| 3880 | GTGCTTACAAAGGCACGC | NC_045512.2 | 3′→5′ | 26207 | 26224 |
| 3881 | TGTGCTTACAAAGGCACG | NC_045512.2 | 3′→5′ | 26208 | 26225 |
| 3882 | TTGTGCTTACAAAGGCAC | NC_045512.2 | 3′→5′ | 26209 | 26226 |
| 3883 | CTTGTGCTTACAAAGGCA | NC_045512.2 | 3′→5′ | 26210 | 26227 |
| 3884 | TACATAAGTTCGTACTCA | NC_045512.2 | 3′→5′ | 26232 | 26249 |
| 3885 | GTACATAAGTTCGTACTC | NC_045512.2 | 3′→5′ | 26233 | 26250 |
| 3886 | AGTACATAAGTTCGTACT | NC_045512.2 | 3′→5′ | 26234 | 26251 |
| 3887 | GAGTACATAAGTTCGTAC | NC_045512.2 | 3′→5′ | 26235 | 26252 |
| 3888 | TGAGTACATAAGTTCGTA | NC_045512.2 | 3′→5′ | 26236 | 26253 |
| 3889 | ATGAGTACATAAGTTCGT | NC_045512.2 | 3′→5′ | 26237 | 26254 |
| 3890 | AATGAGTACATAAGTTCG | NC_045512.2 | 3′→5′ | 26238 | 26255 |
| 3891 | GAATGAGTACATAAGTTC | NC_045512.2 | 3′→5′ | 26239 | 26256 |
| 3892 | CGAATGAGTACATAAGTT | NC_045512.2 | 3′→5′ | 26240 | 26257 |
| 3893 | ACGAATGAGTACATAAGT | NC_045512.2 | 3′→5′ | 26241 | 26258 |
| 3894 | AACGAATGAGTACATAAG | NC_045512.2 | 3′→5′ | 26242 | 26259 |
| 3895 | AAACGAATGAGTACATAA | NC_045512.2 | 3′→5′ | 26243 | 26260 |
| 3896 | GAAACGAATGAGTACATA | NC_045512.2 | 3′→5′ | 26244 | 26261 |
| 3897 | CGAAACGAATGAGTACAT | NC_045512.2 | 3′→5′ | 26245 | 26262 |
| 3898 | CCGAAACGAATGAGTACA | NC_045512.2 | 3′→5′ | 26246 | 26263 |
| 3899 | TCCGAAACGAATGAGTAC | NC_045512.2 | 3′→5′ | 26247 | 26264 |
| 3900 | TTCCGAAACGAATGAGTA | NC_045512.2 | 3′→5′ | 26248 | 26265 |
| 3901 | CTTCCGAAACGAATGAGT | NC_045512.2 | 3′→5′ | 26249 | 26266 |
| 3902 | TCTTCCGAAACGAATGAG | NC_045512.2 | 3′→5′ | 26250 | 26267 |
| 3903 | AACTATTAACGTACCTGT | NC_045512.2 | 3′→5′ | 26269 | 26286 |
| 3904 | TAACTATTAACGTACCTG | NC_045512.2 | 3′→5′ | 26270 | 26287 |
| 3905 | TTAACTATTAACGTACCT | NC_045512.2 | 3′→5′ | 26271 | 26288 |
| 3906 | ATTAACTATTAACGTACC | NC_045512.2 | 3′→5′ | 26272 | 26289 |
| 3907 | TATTAACTATTAACGTAC | NC_045512.2 | 3′→5′ | 26273 | 26290 |
| 3908 | CTATTAACTATTAACGTA | NC_045512.2 | 3′→5′ | 26274 | 26291 |
| 3909 | GCTATTAACTATTAACGT | NC_045512.2 | 3′→5′ | 26275 | 26292 |
| 3910 | CGCTATTAACTATTAACG | NC_045512.2 | 3′→5′ | 26276 | 26293 |
| 3911 | ACGCTATTAACTATTAAC | NC_045512.2 | 3′→5′ | 26277 | 26294 |
| 3912 | TACGCTATTAACTATTAA | NC_045512.2 | 3′→5′ | 26278 | 26295 |
| 3913 | GTACGCTATTAACTATTA | NC_045512.2 | 3′→5′ | 26279 | 26296 |
| 3914 | AGTACGCTATTAACTATT | NC_045512.2 | 3′→5′ | 26280 | 26297 |
| 3915 | AAGTACGCTATTAACTAT | NC_045512.2 | 3′→5′ | 26281 | 26298 |
| 3916 | GAAGTACGCTATTAACTA | NC_045512.2 | 3′→5′ | 26282 | 26299 |
| 3917 | AGAAGTACGCTATTAACT | NC_045512.2 | 3′→5′ | 26283 | 26300 |
| 3918 | AAGAAGTACGCTATTAAC | NC_045512.2 | 3′→5′ | 26284 | 26301 |
| 3919 | AAAGAAGTACGCTATTAA | NC_045512.2 | 3′→5′ | 26285 | 26302 |
| 3920 | AAAAGAAGTACGCTATTA | NC_045512.2 | 3′→5′ | 26286 | 26303 |
| 3921 | AAAAAGAAGTACGCTATT | NC_045512.2 | 3′→5′ | 26287 | 26304 |
| 3922 | GAAAAAGAAGTACGCTAT | NC_045512.2 | 3′→5′ | 26288 | 26305 |
| 3923 | AGAAAAAGAAGTACGCTA | NC_045512.2 | 3′→5′ | 26289 | 26306 |
| 3924 | AAGAAAAAGAAGTACGCT | NC_045512.2 | 3′→5′ | 26290 | 26307 |
| 3925 | CAAGAAAAAGAAGTACGC | NC_045512.2 | 3′→5′ | 26291 | 26308 |
| 3926 | GCAAGAAAAAGAAGTACG | NC_045512.2 | 3′→5′ | 26292 | 26309 |
| 3927 | AGCAAGAAAAAGAAGTAC | NC_045512.2 | 3′→5′ | 26293 | 26310 |
| 3928 | AAGCAAGAAAAAGAAGTA | NC_045512.2 | 3′→5′ | 26294 | 26311 |
| 3929 | AAAGCAAGAAAAAGAAGT | NC_045512.2 | 3′→5′ | 26295 | 26312 |
| 3930 | GAAAGCAAGAAAAAGAAG | NC_045512.2 | 3′→5′ | 26296 | 26313 |
| 3931 | CGAAAGCAAGAAAAAGAA | NC_045512.2 | 3′→5′ | 26297 | 26314 |
| 3932 | ACGAAAGCAAGAAAAAGA | NC_045512.2 | 3′→5′ | 26298 | 26315 |
| 3933 | CACGAAAGCAAGAAAAAG | NC_045512.2 | 3′→5′ | 26299 | 26316 |
| 3934 | CCACGAAAGCAAGAAAAA | NC_045512.2 | 3′→5′ | 26300 | 26317 |
| 3935 | ACCACGAAAGCAAGAAAA | NC_045512.2 | 3′→5′ | 26301 | 26318 |
| 3936 | TACCACGAAAGCAAGAAA | NC_045512.2 | 3′→5′ | 26302 | 26319 |
| 3937 | ATACCACGAAAGCAAGAA | NC_045512.2 | 3′→5′ | 26303 | 26320 |
| 3938 | AATACCACGAAAGCAAGA | NC_045512.2 | 3′→5′ | 26304 | 26321 |
| 3939 | GAATACCACGAAAGCAAG | NC_045512.2 | 3′→5′ | 26305 | 26322 |
| 3940 | AGAATACCACGAAAGCAA | NC_045512.2 | 3′→5′ | 26306 | 26323 |
| 3941 | AAGAATACCACGAAAGCA | NC_045512.2 | 3′→5′ | 26307 | 26324 |
| 3942 | CAAGAATACCACGAAAGC | NC_045512.2 | 3′→5′ | 26308 | 26325 |
| 3943 | GCAAGAATACCACGAAAG | NC_045512.2 | 3′→5′ | 26309 | 26326 |
| 3944 | AGCAAGAATACCACGAAA | NC_045512.2 | 3′→5′ | 26310 | 26327 |
| 3945 | TAGCAAGAATACCACGAA | NC_045512.2 | 3′→5′ | 26311 | 26328 |
| 3946 | CTAGCAAGAATACCACGA | NC_045512.2 | 3′→5′ | 26312 | 26329 |
| 3947 | ACTAGCAAGAATACCACG | NC_045512.2 | 3′→5′ | 26313 | 26330 |
| 3948 | AGTAAGGATGGCTAGTGT | NC_045512.2 | 3′→5′ | 26332 | 26349 |
| 3949 | CAGTAAGGATGGCTAGTG | NC_045512.2 | 3′→5′ | 26333 | 26350 |
| 3950 | GCAGTAAGGATGGCTAGT | NC_045512.2 | 3′→5′ | 26334 | 26351 |
| 3951 | CGCAGTAAGGATGGCTAG | NC_045512.2 | 3′→5′ | 26335 | 26352 |
| 3952 | GCGCAGTAAGGATGGCTA | NC_045512.2 | 3′→5′ | 26336 | 26353 |
| 3953 | AGCGCAGTAAGGATGGCT | NC_045512.2 | 3′→5′ | 26337 | 26354 |
| 3954 | AAGCGCAGTAAGGATGGC | NC_045512.2 | 3′→5′ | 26338 | 26355 |
| 3955 | GAAGCGCAGTAAGGATGG | NC_045512.2 | 3′→5′ | 26339 | 26356 |
| 3956 | CGAAGCGCAGTAAGGATG | NC_045512.2 | 3′→5′ | 26340 | 26357 |
| 3957 | TCGAAGCGCAGTAAGGAT | NC_045512.2 | 3′→5′ | 26341 | 26358 |
| 3958 | ATCGAAGCGCAGTAAGGA | NC_045512.2 | 3′→5′ | 26342 | 26359 |
| 3959 | AATCGAAGCGCAGTAAGG | NC_045512.2 | 3′→5′ | 26343 | 26360 |
| 3960 | CAATCGAAGCGCAGTAAG | NC_045512.2 | 3′→5′ | 26344 | 26361 |
| 3961 | ACAATCGAAGCGCAGTAA | NC_045512.2 | 3′→5′ | 26345 | 26362 |
| 3962 | CACAATCGAAGCGCAGTA | NC_045512.2 | 3′→5′ | 26346 | 26363 |
| 3963 | ACACAATCGAAGCGCAGT | NC_045512.2 | 3′→5′ | 26347 | 26364 |
| 3964 | CACACAATCGAAGCGCAG | NC_045512.2 | 3′→5′ | 26348 | 26365 |
| 3965 | GCACACAATCGAAGCGCA | NC_045512.2 | 3′→5′ | 26349 | 26366 |
| 3966 | CGCACACAATCGAAGCGC | NC_045512.2 | 3′→5′ | 26350 | 26367 |
| 3967 | ACGCACACAATCGAAGCG | NC_045512.2 | 3′→5′ | 26351 | 26368 |
| 3968 | TACGCACACAATCGAAGC | NC_045512.2 | 3′→5′ | 26352 | 26369 |
| 3969 | GTACGCACACAATCGAAG | NC_045512.2 | 3′→5′ | 26353 | 26370 |
| 3970 | AGTACGCACACAATCGAA | NC_045512.2 | 3′→5′ | 26354 | 26371 |
| 3971 | CAGTACGCACACAATCGA | NC_045512.2 | 3′→5′ | 26355 | 26372 |
| 3972 | GCAGTACGCACACAATCG | NC_045512.2 | 3′→5′ | 26356 | 26373 |
| 3973 | AGCAGTACGCACACAATC | NC_045512.2 | 3′→5′ | 26357 | 26374 |
| 3974 | CAGCAGTACGCACACAAT | NC_045512.2 | 3′→5′ | 26358 | 26375 |
| 3975 | GCAGCAGTACGCACACAA | NC_045512.2 | 3′→5′ | 26359 | 26376 |
| 3976 | TGCAGCAGTACGCACACA | NC_045512.2 | 3′→5′ | 26360 | 26377 |
| 3977 | TTGCAGCAGTACGCACAC | NC_045512.2 | 3′→5′ | 26361 | 26378 |
| 3978 | ATTGCAGCAGTACGCACA | NC_045512.2 | 3′→5′ | 26362 | 26379 |
| 3979 | TATTGCAGCAGTACGCAC | NC_045512.2 | 3′→5′ | 26363 | 26380 |
| 3980 | ATATTGCAGCAGTACGCA | NC_045512.2 | 3′→5′ | 26364 | 26381 |
| 3981 | AATATTGCAGCAGTACGC | NC_045512.2 | 3′→5′ | 26365 | 26382 |
| 3982 | CAATATTGCAGCAGTACG | NC_045512.2 | 3′→5′ | 26366 | 26383 |
| 3983 | ACAATATTGCAGCAGTAC | NC_045512.2 | 3′→5′ | 26367 | 26384 |
| 3984 | AACAATATTGCAGCAGTA | NC_045512.2 | 3′→5′ | 26368 | 26385 |
| 3985 | TAACAATATTGCAGCAGT | NC_045512.2 | 3′→5′ | 26369 | 26386 |
| 3986 | TTAACAATATTGCAGCAG | NC_045512.2 | 3′→5′ | 26370 | 26387 |
| 3987 | GTTAACAATATTGCAGCA | NC_045512.2 | 3′→5′ | 26371 | 26388 |
| 3988 | CGTTAACAATATTGCAGC | NC_045512.2 | 3′→5′ | 26372 | 26389 |
| 3989 | ACGTTAACAATATTGCAG | NC_045512.2 | 3′→5′ | 26373 | 26390 |
| 3990 | CACGTTAACAATATTGCA | NC_045512.2 | 3′→5′ | 26374 | 26391 |
| 3991 | TCACGTTAACAATATTGC | NC_045512.2 | 3′→5′ | 26375 | 26392 |
| 3992 | CTCACGTTAACAATATTG | NC_045512.2 | 3′→5′ | 26376 | 26393 |
| 3993 | ACTCACGTTAACAATATT | NC_045512.2 | 3′→5′ | 26377 | 26394 |
| 3994 | CCAGAAGATCAGGAACTC | NC_045512.2 | 3′→5′ | 26450 | 26467 |
| 3995 | ACCAGAAGATCAGGAACT | NC_045512.2 | 3′→5′ | 26451 | 26468 |
| 3996 | GACCAGAAGATCAGGAAC | NC_045512.2 | 3′→5′ | 26452 | 26469 |
| 3997 | AGACCAGAAGATCAGGAA | NC_045512.2 | 3′→5′ | 26453 | 26470 |
| 3998 | TAGACCAGAAGATCAGGA | NC_045512.2 | 3′→5′ | 26454 | 26471 |
| 3999 | TTAGACCAGAAGATCAGG | NC_045512.2 | 3′→5′ | 26455 | 26472 |
| 4000 | TTTAGACCAGAAGATCAG | NC_045512.2 | 3′→5′ | 26456 | 26473 |
| 4001 | GTTTAGACCAGAAGATCA | NC_045512.2 | 3′→5′ | 26457 | 26474 |
| 4002 | CGTTTAGACCAGAAGATC | NC_045512.2 | 3′→5′ | 26458 | 26475 |
| 4003 | TCGTTTAGACCAGAAGAT | NC_045512.2 | 3′→5′ | 26459 | 26476 |
| 4004 | TTCGTTTAGACCAGAAGA | NC_045512.2 | 3′→5′ | 26460 | 26477 |
| 4005 | GTTCGTTTAGACCAGAAG | NC_045512.2 | 3′→5′ | 26461 | 26478 |
| 4006 | AGTTCGTTTAGACCAGAA | NC_045512.2 | 3′→5′ | 26462 | 26479 |
| 4007 | TAGTTCGTTTAGACCAGA | NC_045512.2 | 3′→5′ | 26463 | 26480 |
| 4008 | TTAGTTCGTTTAGACCAG | NC_045512.2 | 3′→5′ | 26464 | 26481 |
| 4009 | TTAAAGTTCCAAACAGAA | NC_045512.2 | 3′→5′ | 26497 | 26514 |
| 4010 | TACTAGGTTCCATTGTTC | NC_045512.2 | 3′→5′ | 26574 | 26591 |
| 4011 | TTACTAGGTTCCATTGTT | NC_045512.2 | 3′→5′ | 26575 | 26592 |
| 4012 | ATTACTAGGTTCCATTGT | NC_045512.2 | 3′→5′ | 26576 | 26593 |
| 4013 | TATTACTAGGTTCCATTG | NC_045512.2 | 3′→5′ | 26577 | 26594 |
| 4014 | CTATTACTAGGTTCCATT | NC_045512.2 | 3′→5′ | 26578 | 26595 |
| 4015 | CCTATTACTAGGTTCCAT | NC_045512.2 | 3′→5′ | 26579 | 26596 |
| 4016 | ACCTATTACTAGGTTCCA | NC_045512.2 | 3′→5′ | 26580 | 26597 |
| 4017 | AACCTATTACTAGGTTCC | NC_045512.2 | 3′→5′ | 26581 | 26598 |
| 4018 | AAACCTATTACTAGGTTC | NC_045512.2 | 3′→5′ | 26582 | 26599 |
| 4019 | GAAACCTATTACTAGGTT | NC_045512.2 | 3′→5′ | 26583 | 26600 |
| 4020 | AAGCGTTCGTGATGTAGC | NC_045512.2 | 3′→5′ | 27033 | 27050 |
| 4021 | AAAGCGTTCGTGATGTAG | NC_045512.2 | 3′→5′ | 27034 | 27051 |
| 4022 | GAAAGCGTTCGTGATGTA | NC_045512.2 | 3′→5′ | 27035 | 27052 |
| 4023 | AGAAAGCGTTCGTGATGT | NC_045512.2 | 3′→5′ | 27036 | 27053 |
| 4024 | AAGAAAGCGTTCGTGATG | NC_045512.2 | 3′→5′ | 27037 | 27054 |
| 4025 | TAAGAAAGCGTTCGTGAT | NC_045512.2 | 3′→5′ | 27038 | 27055 |
| 4026 | ATAAGAAAGCGTTCGTGA | NC_045512.2 | 3′→5′ | 27039 | 27056 |
| 4027 | AATAAGAAAGCGTTCGTG | NC_045512.2 | 3′→5′ | 27040 | 27057 |
| 4028 | TAATAAGAAAGCGTTCGT | NC_045512.2 | 3′→5′ | 27041 | 27058 |
| 4029 | GTAATAAGAAAGCGTTCG | NC_045512.2 | 3′→5′ | 27042 | 27059 |
| 4030 | TGTAATAAGAAAGCGTTC | NC_045512.2 | 3′→5′ | 27043 | 27060 |
| 4031 | TTGTAATAAGAAAGCGTT | NC_045512.2 | 3′→5′ | 27044 | 27061 |
| 4032 | TTTGTAATAAGAAAGCGT | NC_045512.2 | 3′→5′ | 27045 | 27062 |
| 4033 | ATTTGTAATAAGAAAGCG | NC_045512.2 | 3′→5′ | 27046 | 27063 |
| 4034 | AATTTGTAATAAGAAAGC | NC_045512.2 | 3′→5′ | 27047 | 27064 |
| 4035 | GTATGCAGCAAAACCTGA | NC_045512.2 | 3′→5′ | 27093 | 27110 |
| 4036 | TGTATGCAGCAAAACCTG | NC_045512.2 | 3′→5′ | 27094 | 27111 |
| 4037 | TGTTGTCACTTACTGTAC | NC_045512.2 | 3′→5′ | 27183 | 27200 |
| 4038 | CTGTTGTCACTTACTGTA | NC_045512.2 | 3′→5′ | 27184 | 27201 |
| 4039 | TCTGTTGTCACTTACTGT | NC_045512.2 | 3′→5′ | 27185 | 27202 |
| 4040 | ATCTGTTGTCACTTACTG | NC_045512.2 | 3′→5′ | 27186 | 27203 |
| 4041 | CATCTGTTGTCACTTACT | NC_045512.2 | 3′→5′ | 27187 | 27204 |
| 4042 | ACATCTGTTGTCACTTAC | NC_045512.2 | 3′→5′ | 27188 | 27205 |
| 4043 | AACATCTGTTGTCACTTA | NC_045512.2 | 3′→5′ | 27189 | 27206 |
| 4044 | AAACATCTGTTGTCACTT | NC_045512.2 | 3′→5′ | 27190 | 27207 |
| 4045 | GAAACATCTGTTGTCACT | NC_045512.2 | 3′→5′ | 27191 | 27208 |
| 4046 | TGAAACATCTGTTGTCAC | NC_045512.2 | 3′→5′ | 27192 | 27209 |
| 4047 | ATGAAACATCTGTTGTCA | NC_045512.2 | 3′→5′ | 27193 | 27210 |
| 4048 | GATGAAACATCTGTTGTC | NC_045512.2 | 3′→5′ | 27194 | 27211 |
| 4049 | AGATGAAACATCTGTTGT | NC_045512.2 | 3′→5′ | 27195 | 27212 |
| 4050 | TTTCATGTTCGTTTAATC | NC_045512.2 | 3′→5′ | 27382 | 27399 |
| 4051 | TTTTCATGTTCGTTTAAT | NC_045512.2 | 3′→5′ | 27383 | 27400 |
| 4052 | ATTTTCATGTTCGTTTAA | NC_045512.2 | 3′→5′ | 27384 | 27401 |
| 4053 | AATTTTCATGTTCGTTTA | NC_045512.2 | 3′→5′ | 27385 | 27402 |
| 4054 | TAATTTTCATGTTCGTTT | NC_045512.2 | 3′→5′ | 27386 | 27403 |
| 4055 | ATAATTTTCATGTTCGTT | NC_045512.2 | 3′→5′ | 27387 | 27404 |
| 4056 | AATAATTTTCATGTTCGT | NC_045512.2 | 3′→5′ | 27388 | 27405 |
| 4057 | GAATAATTTTCATGTTCG | NC_045512.2 | 3′→5′ | 27389 | 27406 |
| 4058 | AGAATAATTTTCATGTTC | NC_045512.2 | 3′→5′ | 27390 | 27407 |
| 4059 | TGGTGAATTGCCCTCGTA | NC_045512.2 | 3′→5′ | 27511 | 27528 |
| 4060 | ATGGTGAATTGCCCTCGT | NC_045512.2 | 3′→5′ | 27512 | 27529 |
| 4061 | AATGGTGAATTGCCCTCG | NC_045512.2 | 3′→5′ | 27513 | 27530 |
| 4062 | AAATGGTGAATTGCCCTC | NC_045512.2 | 3′→5′ | 27514 | 27531 |
| 4063 | GAAATGGTGAATTGCCCT | NC_045512.2 | 3′→5′ | 27515 | 27532 |
| 4064 | TGAAATGGTGAATTGCCC | NC_045512.2 | 3′→5′ | 27516 | 27533 |
| 4065 | CAAATAGAAGTCAATTAA | NC_045512.2 | 3′→5′ | 27771 | 27788 |
| 4066 | ACAAATAGAAGTCAATTA | NC_045512.2 | 3′→5′ | 27772 | 27789 |
| 4067 | CACAAATAGAAGTCAATT | NC_045512.2 | 3′→5′ | 27773 | 27790 |
| 4068 | GCACAAATAGAAGTCAAT | NC_045512.2 | 3′→5′ | 27774 | 27791 |
| 4069 | AGCACAAATAGAAGTCAA | NC_045512.2 | 3′→5′ | 27775 | 27792 |
| 4070 | AAGCACAAATAGAAGTCA | NC_045512.2 | 3′→5′ | 27776 | 27793 |
| 4071 | AAAGCACAAATAGAAGTC | NC_045512.2 | 3′→5′ | 27777 | 27794 |
| 4072 | AAAAGCACAAATAGAAGT | NC_045512.2 | 3′→5′ | 27778 | 27795 |
| 4073 | AAAAAGCACAAATAGAAG | NC_045512.2 | 3′→5′ | 27779 | 27796 |
| 4074 | TAAAAAGCACAAATAGAA | NC_045512.2 | 3′→5′ | 27780 | 27797 |
| 4075 | CTAAAAAGCACAAATAGA | NC_045512.2 | 3′→5′ | 27781 | 27798 |
| 4076 | GCTAAAAAGCACAAATAG | NC_045512.2 | 3′→5′ | 27782 | 27799 |
| 4077 | GGCTAAAAAGCACAAATA | NC_045512.2 | 3′→5′ | 27783 | 27800 |
| 4078 | AGGCTAAAAAGCACAAAT | NC_045512.2 | 3′→5′ | 27784 | 27801 |
| 4079 | AAGGCTAAAAAGCACAAA | NC_045512.2 | 3′→5′ | 27785 | 27802 |
| 4080 | AAAGGCTAAAAAGCACAA | NC_045512.2 | 3′→5′ | 27786 | 27803 |
| 4081 | GAAAGGCTAAAAAGCACA | NC_045512.2 | 3′→5′ | 27787 | 27804 |
| 4082 | AGAAAGGCTAAAAAGCAC | NC_045512.2 | 3′→5′ | 27788 | 27805 |
| 4083 | CAGAAAGGCTAAAAAGCA | NC_045512.2 | 3′→5′ | 27789 | 27806 |
| 4084 | GCAGAAAGGCTAAAAAGC | NC_045512.2 | 3′→5′ | 27790 | 27807 |
| 4085 | AGCAGAAAGGCTAAAAAG | NC_045512.2 | 3′→5′ | 27791 | 27808 |
| 4086 | TAGCAGAAAGGCTAAAAA | NC_045512.2 | 3′→5′ | 27792 | 27809 |
| 4087 | ATAGCAGAAAGGCTAAAA | NC_045512.2 | 3′→5′ | 27793 | 27810 |
| 4088 | AATAGCAGAAAGGCTAAA | NC_045512.2 | 3′→5′ | 27794 | 27811 |
| 4089 | GAATAGCAGAAAGGCTAA | NC_045512.2 | 3′→5′ | 27795 | 27812 |
| 4090 | GGAATAGCAGAAAGGCTA | NC_045512.2 | 3′→5′ | 27796 | 27813 |
| 4091 | AGGAATAGCAGAAAGGCT | NC_045512.2 | 3′→5′ | 27797 | 27814 |
| 4092 | AAGGAATAGCAGAAAGGC | NC_045512.2 | 3′→5′ | 27798 | 27815 |
| 4093 | CAAGGAATAGCAGAAAGG | NC_045512.2 | 3′→5′ | 27799 | 27816 |
| 4094 | ACAAGGAATAGCAGAAAG | NC_045512.2 | 3′→5′ | 27800 | 27817 |
| 4095 | AACAAGGAATAGCAGAAA | NC_045512.2 | 3′→5′ | 27801 | 27818 |
| 4096 | CCATTATCAGACATTTTA | NC_045512.2 | 3′→5′ | 28270 | 28287 |
| 4097 | TCCATTATCAGACATTTT | NC_045512.2 | 3′→5′ | 28271 | 28288 |
| 4098 | GTCCATTATCAGACATTT | NC_045512.2 | 3′→5′ | 28272 | 28289 |
| 4099 | GGTCCATTATCAGACATT | NC_045512.2 | 3′→5′ | 28273 | 28290 |
| 4100 | GGGTCCATTATCAGACAT | NC_045512.2 | 3′→5′ | 28274 | 28291 |
| 4101 | GGGGTCCATTATCAGACA | NC_045512.2 | 3′→5′ | 28275 | 28292 |
| 4102 | TGGGGTCCATTATCAGAC | NC_045512.2 | 3′→5′ | 28276 | 28293 |
| 4103 | TTGGGGTCCATTATCAGA | NC_045512.2 | 3′→5′ | 28277 | 28294 |
| 4104 | TTTGGGGTCCATTATCAG | NC_045512.2 | 3′→5′ | 28278 | 28295 |
| 4105 | TTTTGGGGTCCATTATCA | NC_045512.2 | 3′→5′ | 28279 | 28296 |
| 4106 | ATTGGGTAAACCTTGGGG | NC_045512.2 | 3′→5′ | 28397 | 28414 |
| 4107 | TATTGGGTAAACCTTGGG | NC_045512.2 | 3′→5′ | 28398 | 28415 |
| 4108 | TTATTGGGTAAACCTTGG | NC_045512.2 | 3′→5′ | 28399 | 28416 |
| 4109 | ATTATTGGGTAAACCTTG | NC_045512.2 | 3′→5′ | 28400 | 28417 |
| 4110 | TATTATTGGGTAAACCTT | NC_045512.2 | 3′→5′ | 28401 | 28418 |
| 4111 | GTATTATTGGGTAAACCT | NC_045512.2 | 3′→5′ | 28402 | 28419 |
| 4112 | AGTATTATTGGGTAAACC | NC_045512.2 | 3′→5′ | 28403 | 28420 |
| 4113 | CAGTATTATTGGGTAAAC | NC_045512.2 | 3′→5′ | 28404 | 28421 |
| 4114 | GCAGTATTATTGGGTAAA | NC_045512.2 | 3′→5′ | 28405 | 28422 |
| 4115 | CGCAGTATTATTGGGTAA | NC_045512.2 | 3′→5′ | 28406 | 28423 |
| 4116 | ACGCAGTATTATTGGGTA | NC_045512.2 | 3′→5′ | 28407 | 28424 |
| 4117 | GACGCAGTATTATTGGGT | NC_045512.2 | 3′→5′ | 28408 | 28425 |
| 4118 | AGACGCAGTATTATTGGG | NC_045512.2 | 3′→5′ | 28409 | 28426 |
| 4119 | AAGACGCAGTATTATTGG | NC_045512.2 | 3′→5′ | 28410 | 28427 |
| 4120 | CAAGACGCAGTATTATTG | NC_045512.2 | 3′→5′ | 28411 | 28428 |
| 4121 | CCAAGACGCAGTATTATT | NC_045512.2 | 3′→5′ | 28412 | 28429 |
| 4122 | ACCAAGACGCAGTATTAT | NC_045512.2 | 3′→5′ | 28413 | 28430 |
| 4123 | AACCAAGACGCAGTATTA | NC_045512.2 | 3′→5′ | 28414 | 28431 |
| 4124 | GAACCAAGACGCAGTATT | NC_045512.2 | 3′→5′ | 28415 | 28432 |
| 4125 | TGAACCAAGACGCAGTAT | NC_045512.2 | 3′→5′ | 28416 | 28433 |
| 4126 | GTGAACCAAGACGCAGTA | NC_045512.2 | 3′→5′ | 28417 | 28434 |
| 4127 | TAGCCAATTTGGTCATCT | NC_045512.2 | 3′→5′ | 28513 | 28530 |
| 4128 | GTAGCCAATTTGGTCATC | NC_045512.2 | 3′→5′ | 28514 | 28531 |
| 4129 | AGTAGCCAATTTGGTCAT | NC_045512.2 | 3′→5′ | 28515 | 28532 |
| 4130 | TAGTAGCCAATTTGGTCA | NC_045512.2 | 3′→5′ | 28516 | 28533 |
| 4131 | GTAGTAGCCAATTTGGTC | NC_045512.2 | 3′→5′ | 28517 | 28534 |
| 4132 | GGTAGTAGCCAATTTGGT | NC_045512.2 | 3′→5′ | 28518 | 28535 |
| 4133 | CGGTAGTAGCCAATTTGG | NC_045512.2 | 3′→5′ | 28519 | 28536 |
| 4134 | TCGGTAGTAGCCAATTTG | NC_045512.2 | 3′→5′ | 28520 | 28537 |
| 4135 | TTCGGTAGTAGCCAATTT | NC_045512.2 | 3′→5′ | 28521 | 28538 |
| 4136 | CTTCGGTAGTAGCCAATT | NC_045512.2 | 3′→5′ | 28522 | 28539 |
| 4137 | TCTTCGGTAGTAGCCAAT | NC_045512.2 | 3′→5′ | 28523 | 28540 |
| 4138 | CTCTTCGGTAGTAGCCAA | NC_045512.2 | 3′→5′ | 28524 | 28541 |
| 4139 | GCTCTTCGGTAGTAGCCA | NC_045512.2 | 3′→5′ | 28525 | 28542 |
| 4140 | AGCTCTTCGGTAGTAGCC | NC_045512.2 | 3′→5′ | 28526 | 28543 |
| 4141 | TAGCTCTTCGGTAGTAGC | NC_045512.2 | 3′→5′ | 28527 | 28544 |
| 4142 | GTAGCTCTTCGGTAGTAG | NC_045512.2 | 3′→5′ | 28528 | 28545 |
| 4143 | GGTAGCTCTTCGGTAGTA | NC_045512.2 | 3′→5′ | 28529 | 28546 |
| 4144 | CAAGGCTCCCTCAGTTGC | NC_045512.2 | 3′→5′ | 28673 | 28690 |
| 4145 | TCAAGGCTCCCTCAGTTG | NC_045512.2 | 3′→5′ | 28674 | 28691 |
| 4146 | TTCAAGGCTCCCTCAGTT | NC_045512.2 | 3′→5′ | 28675 | 28692 |
| 4147 | ATTGCGGGTGCCAATGTG | NC_045512.2 | 3′→5′ | 28706 | 28723 |
| 4148 | GATTGCGGGTGCCAATGT | NC_045512.2 | 3′→5′ | 28707 | 28724 |
| 4149 | GGATTGCGGGTGCCAATG | NC_045512.2 | 3′→5′ | 28708 | 28725 |
| 4150 | AGGATTGCGGGTGCCAAT | NC_045512.2 | 3′→5′ | 28709 | 28726 |
| 4151 | TGAGGAAGTTGTAGCACG | NC_045512.2 | 3′→5′ | 28744 | 28761 |
| 4152 | TTGAGGAAGTTGTAGCAC | NC_045512.2 | 3′→5′ | 28745 | 28762 |
| 4153 | CTTGAGGAAGTTGTAGCA | NC_045512.2 | 3′→5′ | 28746 | 28763 |
| 4154 | CCTTGAGGAAGTTGTAGC | NC_045512.2 | 3′→5′ | 28747 | 28764 |
| 4155 | TCCTTGAGGAAGTTGTAG | NC_045512.2 | 3′→5′ | 28748 | 28765 |
| 4156 | TTCCTTGAGGAAGTTGTA | NC_045512.2 | 3′→5′ | 28749 | 28766 |
| 4157 | GTTCCTTGAGGAAGTTGT | NC_045512.2 | 3′→5′ | 28750 | 28767 |
| 4158 | TGTTCCTTGAGGAAGTTG | NC_045512.2 | 3′→5′ | 28751 | 28768 |
| 4159 | TTGTTCCTTGAGGAAGTT | NC_045512.2 | 3′→5′ | 28752 | 28769 |
| 4160 | GTTGTTCCTTGAGGAAGT | NC_045512.2 | 3′→5′ | 28753 | 28770 |
| 4161 | TGTTGTTCCTTGAGGAAG | NC_045512.2 | 3′→5′ | 28754 | 28771 |
| 4162 | ATGTTGTTCCTTGAGGAA | NC_045512.2 | 3′→5′ | 28755 | 28772 |
| 4163 | AATGTTGTTCCTTGAGGA | NC_045512.2 | 3′→5′ | 28756 | 28773 |
| 4164 | CAATGTTGTTCCTTGAGG | NC_045512.2 | 3′→5′ | 28757 | 28774 |
| 4165 | GCAATGTTGTTCCTTGAG | NC_045512.2 | 3′→5′ | 28758 | 28775 |
| 4166 | GGCAATGTTGTTCCTTGA | NC_045512.2 | 3′→5′ | 28759 | 28776 |
| 4167 | TGGCAATGTTGTTCCTTG | NC_045512.2 | 3′→5′ | 28760 | 28777 |
| 4168 | TTGGCAATGTTGTTCCTT | NC_045512.2 | 3′→5′ | 28761 | 28778 |
| 4169 | TTTGGCAATGTTGTTCCT | NC_045512.2 | 3′→5′ | 28762 | 28779 |
| 4170 | TTTTGGCAATGTTGTTCC | NC_045512.2 | 3′→5′ | 28763 | 28780 |
| 4171 | CTTTTGGCAATGTTGTTC | NC_045512.2 | 3′→5′ | 28764 | 28781 |
| 4172 | CCTTTTGGCAATGTTGTT | NC_045512.2 | 3′→5′ | 28765 | 28782 |
| 4173 | GCCTTTTGGCAATGTTGT | NC_045512.2 | 3′→5′ | 28766 | 28783 |
| 4174 | AGCCTTTTGGCAATGTTG | NC_045512.2 | 3′→5′ | 28767 | 28784 |
| 4175 | AAGCCTTTTGGCAATGTT | NC_045512.2 | 3′→5′ | 28768 | 28785 |
| 4176 | GAAGCCTTTTGGCAATGT | NC_045512.2 | 3′→5′ | 28769 | 28786 |
| 4177 | AGAAGCCTTTTGGCAATG | NC_045512.2 | 3′→5′ | 28770 | 28787 |
| 4178 | TAGAAGCCTTTTGGCAAT | NC_045512.2 | 3′→5′ | 28771 | 28788 |
| 4179 | GTAGAAGCCTTTTGGCAA | NC_045512.2 | 3′→5′ | 28772 | 28789 |
| 4180 | CGTAGAAGCCTTTTGGCA | NC_045512.2 | 3′→5′ | 28773 | 28790 |
| 4181 | GCGTAGAAGCCTTTTGGC | NC_045512.2 | 3′→5′ | 28774 | 28791 |
| 4182 | TGCGTAGAAGCCTTTTGG | NC_045512.2 | 3′→5′ | 28775 | 28792 |
| 4183 | CTGCGTAGAAGCCTTTTG | NC_045512.2 | 3′→5′ | 28776 | 28793 |
| 4184 | TCTGCGTAGAAGCCTTTT | NC_045512.2 | 3′→5′ | 28777 | 28794 |
| 4185 | TTGACTGCCGCCTCTGCT | NC_045512.2 | 3′→5′ | 28799 | 28816 |
| 4186 | CTTGACTGCCGCCTCTGC | NC_045512.2 | 3′→5′ | 28800 | 28817 |
| 4187 | GCTTGACTGCCGCCTCTG | NC_045512.2 | 3′→5′ | 28801 | 28818 |
| 4188 | GGCTTGACTGCCGCCTCT | NC_045512.2 | 3′→5′ | 28802 | 28819 |
| 4189 | AGGCTTGACTGCCGCCTC | NC_045512.2 | 3′→5′ | 28803 | 28820 |
| 4190 | GAGGCTTGACTGCCGCCT | NC_045512.2 | 3′→5′ | 28804 | 28821 |
| 4191 | AGAGGCTTGACTGCCGCC | NC_045512.2 | 3′→5′ | 28805 | 28822 |
| 4192 | AAGAGGCTTGACTGCCGC | NC_045512.2 | 3′→5′ | 28806 | 28823 |
| 4193 | GAAGAGGCTTGACTGCCG | NC_045512.2 | 3′→5′ | 28807 | 28824 |
| 4194 | AGAAGAGGCTTGACTGCC | NC_045512.2 | 3′→5′ | 28808 | 28825 |
| 4195 | GAGAAGAGGCTTGACTGC | NC_045512.2 | 3′→5′ | 28809 | 28826 |
| 4196 | CGAGAAGAGGCTTGACTG | NC_045512.2 | 3′→5′ | 28810 | 28827 |
| 4197 | GGAGTTGAATTTCTTGAA | NC_045512.2 | 3′→5′ | 28852 | 28869 |
| 4198 | AAGCTGGTTCAATCTGTC | NC_045512.2 | 3′→5′ | 28946 | 28963 |
| 4199 | CAAGCTGGTTCAATCTGT | NC_045512.2 | 3′→5′ | 28947 | 28964 |
| 4200 | TCAAGCTGGTTCAATCTG | NC_045512.2 | 3′→5′ | 28948 | 28965 |
| 4201 | CTCAAGCTGGTTCAATCT | NC_045512.2 | 3′→5′ | 28949 | 28966 |
| 4202 | TCTCAAGCTGGTTCAATC | NC_045512.2 | 3′→5′ | 28950 | 28967 |
| 4203 | CTCTCAAGCTGGTTCAAT | NC_045512.2 | 3′→5′ | 28951 | 28968 |
| 4204 | GCTCTCAAGCTGGTTCAA | NC_045512.2 | 3′→5′ | 28952 | 28969 |
| 4205 | TGCTCTCAAGCTGGTTCA | NC_045512.2 | 3′→5′ | 28953 | 28970 |
| 4206 | TTGCTCTCAAGCTGGTTC | NC_045512.2 | 3′→5′ | 28954 | 28971 |
| 4207 | TTTGCTCTCAAGCTGGTT | NC_045512.2 | 3′→5′ | 28955 | 28972 |
| 4208 | TTGTTGGCCTTTACCAGA | NC_045512.2 | 3′→5′ | 28976 | 28993 |
| 4209 | GTTGTTGGCCTTTACCAG | NC_045512.2 | 3′→5′ | 28977 | 28994 |
| 4210 | TGTTGTTGGCCTTTACCA | NC_045512.2 | 3′→5′ | 28978 | 28995 |
| 4211 | TTGTTGTTGGCCTTTACC | NC_045512.2 | 3′→5′ | 28979 | 28996 |
| 4212 | GTTGTTGTTGGCCTTTAC | NC_045512.2 | 3′→5′ | 28980 | 28997 |
| 4213 | TGTTGTTGTTGGCCTTTA | NC_045512.2 | 3′→5′ | 28981 | 28998 |
| 4214 | TTGTTGTTGTTGGCCTTT | NC_045512.2 | 3′→5′ | 28982 | 28999 |
| 4215 | CTTGTTGTTGTTGGCCTT | NC_045512.2 | 3′→5′ | 28983 | 29000 |
| 4216 | CCTTGTTGTTGTTGGCCT | NC_045512.2 | 3′→5′ | 28984 | 29001 |
| 4217 | GCCTTGTTGTTGTTGGCC | NC_045512.2 | 3′→5′ | 28985 | 29002 |
| 4218 | GGCCTTGTTGTTGTTGGC | NC_045512.2 | 3′→5′ | 28986 | 29003 |
| 4219 | TGGCCTTGTTGTTGTTGG | NC_045512.2 | 3′→5′ | 28987 | 29004 |
| 4220 | TTGGCCTTGTTGTTGTTG | NC_045512.2 | 3′→5′ | 28988 | 29005 |
| 4221 | TTTGGCCTTGTTGTTGTT | NC_045512.2 | 3′→5′ | 28989 | 29006 |
| 4222 | GTTTGGCCTTGTTGTTGT | NC_045512.2 | 3′→5′ | 28990 | 29007 |
| 4223 | AGTTTGGCCTTGTTGTTG | NC_045512.2 | 3′→5′ | 28991 | 29008 |
| 4224 | CAGTTTGGCCTTGTTGTT | NC_045512.2 | 3′→5′ | 28992 | 29009 |
| 4225 | ACAGTTTGGCCTTGTTGT | NC_045512.2 | 3′→5′ | 28993 | 29010 |
| 4226 | GACAGTTTGGCCTTGTTG | NC_045512.2 | 3′→5′ | 28994 | 29011 |
| 4227 | TGACAGTTTGGCCTTGTT | NC_045512.2 | 3′→5′ | 28995 | 29012 |
| 4228 | GTGACAGTTTGGCCTTGT | NC_045512.2 | 3′→5′ | 28996 | 29013 |
| 4229 | AGTGACAGTTTGGCCTTG | NC_045512.2 | 3′→5′ | 28997 | 29014 |
| 4230 | TAGTGACAGTTTGGCCTT | NC_045512.2 | 3′→5′ | 28998 | 29015 |
| 4231 | TTAGTGACAGTTTGGCCT | NC_045512.2 | 3′→5′ | 28999 | 29016 |
| 4232 | CTTAGTGACAGTTTGGCC | NC_045512.2 | 3′→5′ | 29000 | 29017 |
| 4233 | TCTTAGTGACAGTTTGGC | NC_045512.2 | 3′→5′ | 29001 | 29018 |
| 4234 | TTCTTAGTGACAGTTTGG | NC_045512.2 | 3′→5′ | 29002 | 29019 |
| 4235 | TTTCTTAGTGACAGTTTG | NC_045512.2 | 3′→5′ | 29003 | 29020 |
| 4236 | ATTTCTTAGTGACAGTTT | NC_045512.2 | 3′→5′ | 29004 | 29021 |
| 4237 | GATTTCTTAGTGACAGTT | NC_045512.2 | 3′→5′ | 29005 | 29022 |
| 4238 | AGATTTCTTAGTGACAGT | NC_045512.2 | 3′→5′ | 29006 | 29023 |
| 4239 | CAGATTTCTTAGTGACAG | NC_045512.2 | 3′→5′ | 29007 | 29024 |
| 4240 | GCAGATTTCTTAGTGACA | NC_045512.2 | 3′→5′ | 29008 | 29025 |
| 4241 | AGCAGATTTCTTAGTGAC | NC_045512.2 | 3′→5′ | 29009 | 29026 |
| 4242 | CAGCAGATTTCTTAGTGA | NC_045512.2 | 3′→5′ | 29010 | 29027 |
| 4243 | GCAGCAGATTTCTTAGTG | NC_045512.2 | 3′→5′ | 29011 | 29028 |
| 4244 | AGCAGCAGATTTCTTAGT | NC_045512.2 | 3′→5′ | 29012 | 29029 |
| 4245 | CAGCAGCAGATTTCTTAG | NC_045512.2 | 3′→5′ | 29013 | 29030 |
| 4246 | TCAGCAGCAGATTTCTTA | NC_045512.2 | 3′→5′ | 29014 | 29031 |
| 4247 | CTCAGCAGCAGATTTCTT | NC_045512.2 | 3′→5′ | 29015 | 29032 |
| 4248 | CCTCAGCAGCAGATTTCT | NC_045512.2 | 3′→5′ | 29016 | 29033 |
| 4249 | GCCTCAGCAGCAGATTTC | NC_045512.2 | 3′→5′ | 29017 | 29034 |
| 4250 | AGTTCCTTGTCTGATTAG | NC_045512.2 | 3′→5′ | 29144 | 29161 |
| 4251 | CAGTTCCTTGTCTGATTA | NC_045512.2 | 3′→5′ | 29145 | 29162 |
| 4252 | TCAGTTCCTTGTCTGATT | NC_045512.2 | 3′→5′ | 29146 | 29163 |
| 4253 | ATCAGTTCCTTGTCTGAT | NC_045512.2 | 3′→5′ | 29147 | 29164 |
| 4254 | AATCAGTTCCTTGTCTGA | NC_045512.2 | 3′→5′ | 29148 | 29165 |
| 4255 | TAATCAGTTCCTTGTCTG | NC_045512.2 | 3′→5′ | 29149 | 29166 |
| 4256 | GTAATCAGTTCCTTGTCT | NC_045512.2 | 3′→5′ | 29150 | 29167 |
| 4257 | TGTAATCAGTTCCTTGTC | NC_045512.2 | 3′→5′ | 29151 | 29168 |
| 4258 | TTGTAATCAGTTCCTTGT | NC_045512.2 | 3′→5′ | 29152 | 29169 |
| 4259 | TTTGTAATCAGTTCCTTG | NC_045512.2 | 3′→5′ | 29153 | 29170 |
| 4260 | GTTTGTAATCAGTTCCTT | NC_045512.2 | 3′→5′ | 29154 | 29171 |
| 4261 | TGTTTGTAATCAGTTCCT | NC_045512.2 | 3′→5′ | 29155 | 29172 |
| 4262 | TTGTGCAATTTGCGGCCA | NC_045512.2 | 3′→5′ | 29174 | 29191 |
| 4263 | ATTGTGCAATTTGCGGCC | NC_045512.2 | 3′→5′ | 29175 | 29192 |
| 4264 | AATTGTGCAATTTGCGGC | NC_045512.2 | 3′→5′ | 29176 | 29193 |
| 4265 | AAATTGTGCAATTTGCGG | NC_045512.2 | 3′→5′ | 29177 | 29194 |
| 4266 | CAAATTGTGCAATTTGCG | NC_045512.2 | 3′→5′ | 29178 | 29195 |
| 4267 | GCAAATTGTGCAATTTGC | NC_045512.2 | 3′→5′ | 29179 | 29196 |
| 4268 | GACTTCCATGCCAATGCG | NC_045512.2 | 3′→5′ | 29228 | 29245 |
| 4269 | TGACTTCCATGCCAATGC | NC_045512.2 | 3′→5′ | 29229 | 29246 |
| 4270 | GTGACTTCCATGCCAATG | NC_045512.2 | 3′→5′ | 29230 | 29247 |
| 4271 | TGTGACTTCCATGCCAAT | NC_045512.2 | 3′→5′ | 29231 | 29248 |
| 4272 | GTGTGACTTCCATGCCAA | NC_045512.2 | 3′→5′ | 29232 | 29249 |
| 4273 | GGTGTGACTTCCATGCCA | NC_045512.2 | 3′→5′ | 29233 | 29250 |
| 4274 | AGGTGTGACTTCCATGCC | NC_045512.2 | 3′→5′ | 29234 | 29251 |
| 4275 | AAGGTGTGACTTCCATGC | NC_045512.2 | 3′→5′ | 29235 | 29252 |
| 4276 | GAAGGTGTGACTTCCATG | NC_045512.2 | 3′→5′ | 29236 | 29253 |
| 4277 | CGAAGGTGTGACTTCCAT | NC_045512.2 | 3′→5′ | 29237 | 29254 |
| 4278 | CCGAAGGTGTGACTTCCA | NC_045512.2 | 3′→5′ | 29238 | 29255 |
| 4279 | CCCGAAGGTGTGACTTCC | NC_045512.2 | 3′→5′ | 29239 | 29256 |
| 4280 | TCCCGAAGGTGTGACTTC | NC_045512.2 | 3′→5′ | 29240 | 29257 |
| 4281 | TTCCCGAAGGTGTGACTT | NC_045512.2 | 3′→5′ | 29241 | 29258 |
| 4282 | GTTCCCGAAGGTGTGACT | NC_045512.2 | 3′→5′ | 29242 | 29259 |
| 4283 | ATCTTTGTCATCCAATTT | NC_045512.2 | 3′→5′ | 29285 | 29302 |
| 4284 | GATCTTTGTCATCCAATT | NC_045512.2 | 3′→5′ | 29286 | 29303 |
| 4285 | GGATCTTTGTCATCCAAT | NC_045512.2 | 3′→5′ | 29287 | 29304 |
| 4286 | TGGATCTTTGTCATCCAA | NC_045512.2 | 3′→5′ | 29288 | 29305 |
| 4287 | TGTTTTGTATGCGTCAAT | NC_045512.2 | 3′→5′ | 29342 | 29359 |
| 4288 | ATGTTTTGTATGCGTCAA | NC_045512.2 | 3′→5′ | 29343 | 29360 |
| 4289 | AATGTTTTGTATGCGTCA | NC_045512.2 | 3′→5′ | 29344 | 29361 |
| 4290 | GAATGTTTTGTATGCGTC | NC_045512.2 | 3′→5′ | 29345 | 29362 |
| 4291 | GGAATGTTTTGTATGCGT | NC_045512.2 | 3′→5′ | 29346 | 29363 |
| 4292 | GGGAATGTTTTGTATGCG | NC_045512.2 | 3′→5′ | 29347 | 29364 |
| 4293 | TGGGAATGTTTTGTATGC | NC_045512.2 | 3′→5′ | 29348 | 29365 |
| 4294 | GTGGGAATGTTTTGTATG | NC_045512.2 | 3′→5′ | 29349 | 29366 |
| 4295 | GGTGGGAATGTTTTGTAT | NC_045512.2 | 3′→5′ | 29350 | 29367 |
| 4296 | TGGTGGGAATGTTTTGTA | NC_045512.2 | 3′→5′ | 29351 | 29368 |
| 4297 | TTGGTGGGAATGTTTTGT | NC_045512.2 | 3′→5′ | 29352 | 29369 |
| 4298 | GTTGGTGGGAATGTTTTG | NC_045512.2 | 3′→5′ | 29353 | 29370 |
| 4299 | TGTTGGTGGGAATGTTTT | NC_045512.2 | 3′→5′ | 29354 | 29371 |
| 4300 | CTGTTGGTGGGAATGTTT | NC_045512.2 | 3′→5′ | 29355 | 29372 |
| 4301 | TCTGTTGGTGGGAATGTT | NC_045512.2 | 3′→5′ | 29356 | 29373 |
| 4302 | CTCTGTTGGTGGGAATGT | NC_045512.2 | 3′→5′ | 29357 | 29374 |
| 4303 | GCTCTGTTGGTGGGAATG | NC_045512.2 | 3′→5′ | 29358 | 29375 |
| 4304 | GGCTCTGTTGGTGGGAAT | NC_045512.2 | 3′→5′ | 29359 | 29376 |
| 4305 | AGGCTCTGTTGGTGGGAA | NC_045512.2 | 3′→5′ | 29360 | 29377 |
| 4306 | TAGGCTCTGTTGGTGGGA | NC_045512.2 | 3′→5′ | 29361 | 29378 |
| 4307 | TTAGGCTCTGTTGGTGGG | NC_045512.2 | 3′→5′ | 29362 | 29379 |
| 4308 | TTTAGGCTCTGTTGGTGG | NC_045512.2 | 3′→5′ | 29363 | 29380 |
| 4309 | TTTTAGGCTCTGTTGGTG | NC_045512.2 | 3′→5′ | 29364 | 29381 |
| 4310 | TTTTTAGGCTCTGTTGGT | NC_045512.2 | 3′→5′ | 29365 | 29382 |
| 4311 | CTTTTTAGGCTCTGTTGG | NC_045512.2 | 3′→5′ | 29366 | 29383 |
| 4312 | CCTTTTTAGGCTCTGTTG | NC_045512.2 | 3′→5′ | 29367 | 29384 |
| 4313 | TCCTTTTTAGGCTCTGTT | NC_045512.2 | 3′→5′ | 29368 | 29385 |
| 4314 | GTCCTTTTTAGGCTCTGT | NC_045512.2 | 3′→5′ | 29369 | 29386 |
| 4315 | TGTCCTTTTTAGGCTCTG | NC_045512.2 | 3′→5′ | 29370 | 29387 |
| 4316 | TTGTCCTTTTTAGGCTCT | NC_045512.2 | 3′→5′ | 29371 | 29388 |
| 4317 | TTTGTCCTTTTTAGGCTC | NC_045512.2 | 3′→5′ | 29372 | 29389 |
| 4318 | TTTTGTCCTTTTTAGGCT | NC_045512.2 | 3′→5′ | 29373 | 29390 |
| 4319 | TTTTTGTCCTTTTTAGGC | NC_045512.2 | 3′→5′ | 29374 | 29391 |
| 4320 | CTTTTTGTCCTTTTTAGG | NC_045512.2 | 3′→5′ | 29375 | 29392 |
| 4321 | TCTTTTTGTCCTTTTTAG | NC_045512.2 | 3′→5′ | 29376 | 29393 |
| 4322 | TTCTTTTTGTCCTTTTTA | NC_045512.2 | 3′→5′ | 29377 | 29394 |
| 4323 | AGGAAGAAGAGTCACAGT | NC_045512.2 | 3′→5′ | 29444 | 29461 |
| 4324 | CAGGAAGAAGAGTCACAG | NC_045512.2 | 3′→5′ | 29445 | 29462 |
| 4325 | GCAGGAAGAAGAGTCACA | NC_045512.2 | 3′→5′ | 29446 | 29463 |
| 4326 | CATCTGCCTTGTGTGGTC | NC_045512.2 | 3′→5′ | 29543 | 29560 |
| 4327 | CCATCTGCCTTGTGTGGT | NC_045512.2 | 3′→5′ | 29544 | 29561 |
| 4328 | CCCATCTGCCTTGTGTGG | NC_045512.2 | 3′→5′ | 29545 | 29562 |
| 4329 | GCCCATCTGCCTTGTGTG | NC_045512.2 | 3′→5′ | 29546 | 29563 |
| 4330 | AGCCCATCTGCCTTGTGT | NC_045512.2 | 3′→5′ | 29547 | 29564 |
| 4331 | TAGCCCATCTGCCTTGTG | NC_045512.2 | 3′→5′ | 29548 | 29565 |
| 4332 | ATAGCCCATCTGCCTTGT | NC_045512.2 | 3′→5′ | 29549 | 29566 |
| 4333 | TGCACAAGAGTAGACTAT | NC_045512.2 | 3′→5′ | 29598 | 29615 |
| 4334 | CTGCACAAGAGTAGACTA | NC_045512.2 | 3′→5′ | 29599 | 29616 |
| 4335 | TCTGCACAAGAGTAGACT | NC_045512.2 | 3′→5′ | 29600 | 29617 |
| 4336 | TTCTGCACAAGAGTAGAC | NC_045512.2 | 3′→5′ | 29601 | 29618 |
| 4337 | ATTCTGCACAAGAGTAGA | NC_045512.2 | 3′→5′ | 29602 | 29619 |
| 4338 | CATTCTGCACAAGAGTAG | NC_045512.2 | 3′→5′ | 29603 | 29620 |
| 4339 | TCATTCTGCACAAGAGTA | NC_045512.2 | 3′→5′ | 29604 | 29621 |
| 4340 | TTCATTCTGCACAAGAGT | NC_045512.2 | 3′→5′ | 29605 | 29622 |
| 4341 | ATTCATTCTGCACAAGAG | NC_045512.2 | 3′→5′ | 29606 | 29623 |
| 4342 | AATTCATTCTGCACAAGA | NC_045512.2 | 3′→5′ | 29607 | 29624 |
| 4343 | GAATTCATTCTGCACAAG | NC_045512.2 | 3′→5′ | 29608 | 29625 |
| 4344 | AGAATTCATTCTGCACAA | NC_045512.2 | 3′→5′ | 29609 | 29626 |
| 4345 | GAGAATTCATTCTGCACA | NC_045512.2 | 3′→5′ | 29610 | 29627 |
| 4346 | CGAGAATTCATTCTGCAC | NC_045512.2 | 3′→5′ | 29611 | 29628 |
| 4347 | ACGAGAATTCATTCTGCA | NC_045512.2 | 3′→5′ | 29612 | 29629 |
| 4348 | TACGAGAATTCATTCTGC | NC_045512.2 | 3′→5′ | 29613 | 29630 |
| 4349 | TGAGATTAAAGTTAACTA | NC_045512.2 | 3′→5′ | 29652 | 29669 |
| 4350 | GTGAGATTAAAGTTAACT | NC_045512.2 | 3′→5′ | 29653 | 29670 |
| 4351 | TGTGAGATTAAAGTTAAC | NC_045512.2 | 3′→5′ | 29654 | 29671 |
| 4352 | ATGTGAGATTAAAGTTAA | NC_045512.2 | 3′→5′ | 29655 | 29672 |
| 4353 | TATGTGAGATTAAAGTTA | NC_045512.2 | 3′→5′ | 29656 | 29673 |
| 4354 | CTATGTGAGATTAAAGTT | NC_045512.2 | 3′→5′ | 29657 | 29674 |
| 4355 | GCTATGTGAGATTAAAGT | NC_045512.2 | 3′→5′ | 29658 | 29675 |
| 4356 | TGCTATGTGAGATTAAAG | NC_045512.2 | 3′→5′ | 29659 | 29676 |
| 4357 | TTGCTATGTGAGATTAAA | NC_045512.2 | 3′→5′ | 29660 | 29677 |
| 4358 | ATTGCTATGTGAGATTAA | NC_045512.2 | 3′→5′ | 29661 | 29678 |
| 4359 | GATTGCTATGTGAGATTA | NC_045512.2 | 3′→5′ | 29662 | 29679 |
| 4360 | AGATTGCTATGTGAGATT | NC_045512.2 | 3′→5′ | 29663 | 29680 |
| 4361 | AAGATTGCTATGTGAGAT | NC_045512.2 | 3′→5′ | 29664 | 29681 |
| 4362 | AAAGATTGCTATGTGAGA | NC_045512.2 | 3′→5′ | 29665 | 29682 |
| 4363 | TAAAGATTGCTATGTGAG | NC_045512.2 | 3′→5′ | 29666 | 29683 |
| 4364 | TTAAAGATTGCTATGTGA | NC_045512.2 | 3′→5′ | 29667 | 29684 |
| 4365 | ATTAAAGATTGCTATGTG | NC_045512.2 | 3′→5′ | 29668 | 29685 |
| 4366 | GATTAAAGATTGCTATGT | NC_045512.2 | 3′→5′ | 29669 | 29686 |
| 4367 | TGATTAAAGATTGCTATG | NC_045512.2 | 3′→5′ | 29670 | 29687 |
| 4368 | CCTCCCTAATGTTACACA | NC_045512.2 | 3′→5′ | 29689 | 29706 |
| 4369 | TCCTCCCTAATGTTACAC | NC_045512.2 | 3′→5′ | 29690 | 29707 |
| 4370 | GTCCTCCCTAATGTTACA | NC_045512.2 | 3′→5′ | 29691 | 29708 |
| 4371 | AGTCCTCCCTAATGTTAC | NC_045512.2 | 3′→5′ | 29692 | 29709 |
| 4372 | AAGTCCTCCCTAATGTTA | NC_045512.2 | 3′→5′ | 29693 | 29710 |
| 4373 | CAAGTCCTCCCTAATGTT | NC_045512.2 | 3′→5′ | 29694 | 29711 |
| 4374 | TCAAGTCCTCCCTAATGT | NC_045512.2 | 3′→5′ | 29695 | 29712 |
| 4375 | TTCAAGTCCTCCCTAATG | NC_045512.2 | 3′→5′ | 29696 | 29713 |
| 4376 | TTTCAAGTCCTCCCTAAT | NC_045512.2 | 3′→5′ | 29697 | 29714 |
| 4377 | CTTTCAAGTCCTCCCTAA | NC_045512.2 | 3′→5′ | 29698 | 29715 |
| 4378 | TCTTTCAAGTCCTCCCTA | NC_045512.2 | 3′→5′ | 29699 | 29716 |
| 4379 | CTCTTTCAAGTCCTCCCT | NC_045512.2 | 3′→5′ | 29700 | 29717 |
| 4380 | GCTCTTTCAAGTCCTCCC | NC_045512.2 | 3′→5′ | 29701 | 29718 |
| 4381 | GGCTCTTTCAAGTCCTCC | NC_045512.2 | 3′→5′ | 29702 | 29719 |
| 4382 | TGGCTCTTTCAAGTCCTC | NC_045512.2 | 3′→5′ | 29703 | 29720 |
| 4383 | GTGGCTCTTTCAAGTCCT | NC_045512.2 | 3′→5′ | 29704 | 29721 |
| 4384 | GGTGGCTCTTTCAAGTCC | NC_045512.2 | 3′→5′ | 29705 | 29722 |
| 4385 | TGGTGGCTCTTTCAAGTC | NC_045512.2 | 3′→5′ | 29706 | 29723 |
| 4386 | GTGGTGGCTCTTTCAAGT | NC_045512.2 | 3′→5′ | 29707 | 29724 |
| 4387 | TGTGGTGGCTCTTTCAAG | NC_045512.2 | 3′→5′ | 29708 | 29725 |
| 4388 | ATGTGGTGGCTCTTTCAA | NC_045512.2 | 3′→5′ | 29709 | 29726 |
| 4389 | AATGTGGTGGCTCTTTCA | NC_045512.2 | 3′→5′ | 29710 | 29727 |
| 4390 | AAATGTGGTGGCTCTTTC | NC_045512.2 | 3′→5′ | 29711 | 29728 |
| 4391 | AAAATGTGGTGGCTCTTT | NC_045512.2 | 3′→5′ | 29712 | 29729 |
| 4392 | GAAAATGTGGTGGCTCTT | NC_045512.2 | 3′→5′ | 29713 | 29730 |
| 4393 | TGAAAATGTGGTGGCTCT | NC_045512.2 | 3′→5′ | 29714 | 29731 |
| 4394 | GTACTCCGCGTGGCCTCG | NC_045512.2 | 3′→5′ | 29733 | 29750 |
| 4395 | CGTACTCCGCGTGGCCTC | NC_045512.2 | 3′→5′ | 29734 | 29751 |
| 4396 | TCGTACTCCGCGTGGCCT | NC_045512.2 | 3′→5′ | 29735 | 29752 |
| 4397 | ATCGTACTCCGCGTGGCC | NC_045512.2 | 3′→5′ | 29736 | 29753 |
| 4398 | GATCGTACTCCGCGTGGC | NC_045512.2 | 3′→5′ | 29737 | 29754 |
| 4399 | CGATCGTACTCCGCGTGG | NC_045512.2 | 3′→5′ | 29738 | 29755 |
| 4400 | TCGATCGTACTCCGCGTG | NC_045512.2 | 3′→5′ | 29739 | 29756 |
| 4401 | CTCGATCGTACTCCGCGT | NC_045512.2 | 3′→5′ | 29740 | 29757 |
| 4402 | GCAGCTCTCCCTAGCATT | NC_045512.2 | 3′→5′ | 29770 | 29787 |
| 4403 | GGCAGCTCTCCCTAGCAT | NC_045512.2 | 3′→5′ | 29771 | 29788 |
| 4404 | AGGCAGCTCTCCCTAGCA | NC_045512.2 | 3′→5′ | 29772 | 29789 |
| 4405 | TAGGCAGCTCTCCCTAGC | NC_045512.2 | 3′→5′ | 29773 | 29790 |
| 4406 | ATAGGCAGCTCTCCCTAG | NC_045512.2 | 3′→5′ | 29774 | 29791 |
| 4407 | TATAGGCAGCTCTCCCTA | NC_045512.2 | 3′→5′ | 29775 | 29792 |
| 4408 | ATATAGGCAGCTCTCCCT | NC_045512.2 | 3′→5′ | 29776 | 29793 |
| 4409 | CATATAGGCAGCTCTCCC | NC_045512.2 | 3′→5′ | 29777 | 29794 |
| 4410 | CCATATAGGCAGCTCTCC | NC_045512.2 | 3′→5′ | 29778 | 29795 |
| 4411 | TCCATATAGGCAGCTCTC | NC_045512.2 | 3′→5′ | 29779 | 29796 |
| 4412 | TTCCATATAGGCAGCTCT | NC_045512.2 | 3′→5′ | 29780 | 29797 |
| 4413 | CTTCCATATAGGCAGCTC | NC_045512.2 | 3′→5′ | 29781 | 29798 |
| 4414 | TCTTCCATATAGGCAGCT | NC_045512.2 | 3′→5′ | 29782 | 29799 |
| 4415 | CTCTTCCATATAGGCAGC | NC_045512.2 | 3′→5′ | 29783 | 29800 |
| 4416 | GCTCTTCCATATAGGCAG | NC_045512.2 | 3′→5′ | 29784 | 29801 |
| 4417 | GGCTCTTCCATATAGGCA | NC_045512.2 | 3′→5′ | 29785 | 29802 |
| 4418 | GGGCTCTTCCATATAGGC | NC_045512.2 | 3′→5′ | 29786 | 29803 |
| 4419 | AGGGCTCTTCCATATAGG | NC_045512.2 | 3′→5′ | 29787 | 29804 |
| 4420 | TAGGGCTCTTCCATATAG | NC_045512.2 | 3′→5′ | 29788 | 29805 |
| 4421 | TTAGGGCTCTTCCATATA | NC_045512.2 | 3′→5′ | 29789 | 29806 |
| 4422 | ATTAGGGCTCTTCCATAT | NC_045512.2 | 3′→5′ | 29790 | 29807 |
| 4423 | CATTAGGGCTCTTCCATA | NC_045512.2 | 3′→5′ | 29791 | 29808 |
| 4424 | ACATTAGGGCTCTTCCAT | NC_045512.2 | 3′→5′ | 29792 | 29809 |
| 4425 | CACATTAGGGCTCTTCCA | NC_045512.2 | 3′→5′ | 29793 | 29810 |
| 4426 | ACACATTAGGGCTCTTCC | NC_045512.2 | 3′→5′ | 29794 | 29811 |
| 4427 | TACACATTAGGGCTCTTC | NC_045512.2 | 3′→5′ | 29795 | 29812 |
| 4428 | TTACACATTAGGGCTCTT | NC_045512.2 | 3′→5′ | 29796 | 29813 |
| 4429 | TTTACACATTAGGGCTCT | NC_045512.2 | 3′→5′ | 29797 | 29814 |
| 4430 | TTTTACACATTAGGGCTC | NC_045512.2 | 3′→5′ | 29798 | 29815 |
| 4431 | ATTTTACACATTAGGGCT | NC_045512.2 | 3′→5′ | 29799 | 29816 |
| 4432 | AATTTTACACATTAGGGC | NC_045512.2 | 3′→5′ | 29800 | 29817 |
| 4433 | TAATTTTACACATTAGGG | NC_045512.2 | 3′→5′ | 29801 | 29818 |
| 4434 | TTAATTTTACACATTAGG | NC_045512.2 | 3′→5′ | 29802 | 29819 |
| 4435 | ATTAATTTTACACATTAG | NC_045512.2 | 3′→5′ | 29803 | 29820 |
| 4436 | AATTAATTTTACACATTA | NC_045512.2 | 3′→5′ | 29804 | 29821 |
| 4437 | AAATTAATTTTACACATT | NC_045512.2 | 3′→5′ | 29805 | 29822 |
| 4438 | AAAATTAATTTTACACAT | NC_045512.2 | 3′→5′ | 29806 | 29823 |
| 4439 | TAAAATTAATTTTACACA | NC_045512.2 | 3′→5′ | 29807 | 29824 |
| 4440 | CTAAAATTAATTTTACAC | NC_045512.2 | 3′→5′ | 29808 | 29825 |
| 4441 | ACTAAAATTAATTTTACA | NC_045512.2 | 3′→5′ | 29809 | 29826 |
| 4442 | TACTAAAATTAATTTTAC | NC_045512.2 | 3′→5′ | 29810 | 29827 |
| 4443 | CTACTAAAATTAATTTTA | NC_045512.2 | 3′→5′ | 29811 | 29828 |
| 4444 | ACGGACGAAACCGTAAGCAG | NC_045512.2 | 3′→5′ | 190 | 209 |
| 4445 | CACGGACGAAACCGTAAGCA | NC_045512.2 | 3′→5′ | 191 | 210 |
| 4446 | ACACGGACGAAACCGTAAGC | NC_045512.2 | 3′→5′ | 192 | 211 |
| 4447 | AACACGGACGAAACCGTAAG | NC_045512.2 | 3′→5′ | 193 | 212 |
| 4448 | CAACACGGACGAAACCGTAA | NC_045512.2 | 3′→5′ | 194 | 213 |
| 4449 | GCAACACGGACGAAACCGTA | NC_045512.2 | 3′→5′ | 195 | 214 |
| 4450 | TGCAACACGGACGAAACCGT | NC_045512.2 | 3′→5′ | 196 | 215 |
| 4451 | CTGCAACACGGACGAAACCG | NC_045512.2 | 3′→5′ | 197 | 216 |
| 4452 | CACACCCGGACGAAACCTAG | NC_045512.2 | 3′→5′ | 233 | 252 |
| 4453 | TCACACCCGGACGAAACCTA | NC_045512.2 | 3′→5′ | 234 | 253 |
| 4454 | GTCACACCCGGACGAAACCT | NC_045512.2 | 3′→5′ | 235 | 254 |
| 4455 | GGTCACACCCGGACGAAACC | NC_045512.2 | 3′→5′ | 236 | 255 |
| 4456 | CGGTCACACCCGGACGAAAC | NC_045512.2 | 3′→5′ | 237 | 256 |
| 4457 | TCGGTCACACCCGGACGAAA | NC_045512.2 | 3′→5′ | 238 | 257 |
| 4458 | TTCGGTCACACCCGGACGAA | NC_045512.2 | 3′→5′ | 239 | 258 |
| 4459 | TTTCGGTCACACCCGGACGA | NC_045512.2 | 3′→5′ | 240 | 259 |
| 4460 | CTTTCGGTCACACCCGGACG | NC_045512.2 | 3′→5′ | 241 | 260 |
| 4461 | CCTTTCGGTCACACCCGGAC | NC_045512.2 | 3′→5′ | 242 | 261 |
| 4462 | ACCTTTCGGTCACACCCGGA | NC_045512.2 | 3′→5′ | 243 | 262 |
| 4463 | TACCTTTCGGTCACACCCGG | NC_045512.2 | 3′→5′ | 244 | 263 |
| 4464 | TTACCTTTCGGTCACACCCG | NC_045512.2 | 3′→5′ | 245 | 264 |
| 4465 | CTTACCTTTCGGTCACACCC | NC_045512.2 | 3′→5′ | 246 | 265 |
| 4466 | TCTTACCTTTCGGTCACACC | NC_045512.2 | 3′→5′ | 247 | 266 |
| 4467 | ATCTTACCTTTCGGTCACAC | NC_045512.2 | 3′→5′ | 248 | 267 |
| 4468 | CATCTTACCTTTCGGTCACA | NC_045512.2 | 3′→5′ | 249 | 268 |
| 4469 | CCATCTTACCTTTCGGTCAC | NC_045512.2 | 3′→5′ | 250 | 269 |
| 4470 | TCCATCTTACCTTTCGGTCA | NC_045512.2 | 3′→5′ | 251 | 270 |
| 4471 | CTCCATCTTACCTTTCGGTC | NC_045512.2 | 3′→5′ | 252 | 271 |
| 4472 | TCTCCATCTTACCTTTCGGT | NC_045512.2 | 3′→5′ | 253 | 272 |
| 4473 | CTCTCCATCTTACCTTTCGG | NC_045512.2 | 3′→5′ | 254 | 273 |
| 4474 | GCTCTCCATCTTACCTTTCG | NC_045512.2 | 3′→5′ | 255 | 274 |
| 4475 | GGCTCTCCATCTTACCTTTC | NC_045512.2 | 3′→5′ | 256 | 275 |
| 4476 | AGGCTCTCCATCTTACCTTT | NC_045512.2 | 3′→5′ | 257 | 276 |
| 4477 | AAGGCTCTCCATCTTACCTT | NC_045512.2 | 3′→5′ | 258 | 277 |
| 4478 | CAAGGCTCTCCATCTTACCT | NC_045512.2 | 3′→5′ | 259 | 278 |
| 4479 | ACAAGGCTCTCCATCTTACC | NC_045512.2 | 3′→5′ | 260 | 279 |
| 4480 | GACGTGTGTTTTCTCGTTGA | NC_045512.2 | 3′→5′ | 288 | 307 |
| 4481 | GGACGTGTGTTTTCTCGTTG | NC_045512.2 | 3′→5′ | 289 | 308 |
| 4482 | TGGACGTGTGTTTTCTCGTT | NC_045512.2 | 3′→5′ | 290 | 309 |
| 4483 | TTGGACGTGTGTTTTCTCGT | NC_045512.2 | 3′→5′ | 291 | 310 |
| 4484 | GTTGGACGTGTGTTTTCTCG | NC_045512.2 | 3′→5′ | 292 | 311 |
| 4485 | AGTTGGACGTGTGTTTTCTC | NC_045512.2 | 3′→5′ | 293 | 312 |
| 4486 | GAGTTGGACGTGTGTTTTCT | NC_045512.2 | 3′→5′ | 294 | 313 |
| 4487 | TGAGTTGGACGTGTGTTTTC | NC_045512.2 | 3′→5′ | 295 | 314 |
| 4488 | CTGAGTTGGACGTGTGTTTT | NC_045512.2 | 3′→5′ | 296 | 315 |
| 4489 | ACTGAGTTGGACGTGTGTTT | NC_045512.2 | 3′→5′ | 297 | 316 |
| 4490 | AACTGAGTTGGACGTGTGTT | NC_045512.2 | 3′→5′ | 298 | 317 |
| 4491 | AAACTGAGTTGGACGTGTGT | NC_045512.2 | 3′→5′ | 299 | 318 |
| 4492 | CAAACTGAGTTGGACGTGTG | NC_045512.2 | 3′→5′ | 300 | 319 |
| 4493 | GCAAACTGAGTTGGACGTGT | NC_045512.2 | 3′→5′ | 301 | 320 |
| 4494 | GGCAAACTGAGTTGGACGTG | NC_045512.2 | 3′→5′ | 302 | 321 |
| 4495 | AGGCAAACTGAGTTGGACGT | NC_045512.2 | 3′→5′ | 303 | 322 |
| 4496 | CAGGCAAACTGAGTTGGACG | NC_045512.2 | 3′→5′ | 304 | 323 |
| 4497 | ACAGGCAAACTGAGTTGGAC | NC_045512.2 | 3′→5′ | 305 | 324 |
| 4498 | AACACATAGGGCTGTTCAAG | NC_045512.2 | 3′→5′ | 455 | 474 |
| 4499 | GAACACATAGGGCTGTTCAA | NC_045512.2 | 3′→5′ | 456 | 475 |
| 4500 | TGAACACATAGGGCTGTTCA | NC_045512.2 | 3′→5′ | 457 | 476 |
| 4501 | ATGAACACATAGGGCTGTTC | NC_045512.2 | 3′→5′ | 458 | 477 |
| 4502 | CCGTTCTTACGAAGAAGAAC | NC_045512.2 | 3′→5′ | 626 | 645 |
| 4503 | ACCGTTCTTACGAAGAAGAA | NC_045512.2 | 3′→5′ | 627 | 646 |
| 4504 | TACCGTTCTTACGAAGAAGA | NC_045512.2 | 3′→5′ | 628 | 647 |
| 4505 | TTACCGTTCTTACGAAGAAG | NC_045512.2 | 3′→5′ | 629 | 648 |
| 4506 | ATTACCGTTCTTACGAAGAA | NC_045512.2 | 3′→5′ | 630 | 649 |
| 4507 | TATTACCGTTCTTACGAAGA | NC_045512.2 | 3′→5′ | 631 | 650 |
| 4508 | TTATTACCGTTCTTACGAAG | NC_045512.2 | 3′→5′ | 632 | 651 |
| 4509 | GGATCAGTGCCAAGCTCGTC | NC_045512.2 | 3′→5′ | 704 | 723 |
| 4510 | CAGTAAGTTTTAAATAACCA | NC_045512.2 | 3′→5′ | 3352 | 3371 |
| 4511 | TCAGTAAGTTTTAAATAACC | NC_045512.2 | 3′→5′ | 3353 | 3372 |
| 4512 | GTCAGTAAGTTTTAAATAAC | NC_045512.2 | 3′→5′ | 3354 | 3373 |
| 4513 | TGTCAGTAAGTTTTAAATAA | NC_045512.2 | 3′→5′ | 3355 | 3374 |
| 4514 | TTGTCAGTAAGTTTTAAATA | NC_045512.2 | 3′→5′ | 3356 | 3375 |
| 4515 | ATTGTCAGTAAGTTTTAAAT | NC_045512.2 | 3′→5′ | 3357 | 3376 |
| 4516 | CATTGTCAGTAAGTTTTAAA | NC_045512.2 | 3′→5′ | 3358 | 3377 |
| 4517 | ACATTGTCAGTAAGTTTTAA | NC_045512.2 | 3′→5′ | 3359 | 3378 |
| 4518 | AGTGCACAAAAGTTAGCAGC | NC_045512.2 | 3′→5′ | 5384 | 5403 |
| 4519 | TTTCCACTACTTCTTCAGAG | NC_045512.2 | 3′→5′ | 6406 | 6425 |
| 4520 | TTTTCCACTACTTCTTCAGA | NC_045512.2 | 3′→5′ | 6407 | 6426 |
| 4521 | ATTTTCCACTACTTCTTCAG | NC_045512.2 | 3′→5′ | 6408 | 6427 |
| 4522 | GATTTTCCACTACTTCTTCA | NC_045512.2 | 3′→5′ | 6409 | 6428 |
| 4523 | GGATTTTCCACTACTTCTTC | NC_045512.2 | 3′→5′ | 6410 | 6429 |
| 4524 | AGGATTTTCCACTACTTCTT | NC_045512.2 | 3′→5′ | 6411 | 6430 |
| 4525 | TAGGATTTTCCACTACTTCT | NC_045512.2 | 3′→5′ | 6412 | 6431 |
| 4526 | GTAGGATTTTCCACTACTTC | NC_045512.2 | 3′→5′ | 6413 | 6432 |
| 4527 | GGTAGGATTTTCCACTACTT | NC_045512.2 | 3′→5′ | 6414 | 6433 |
| 4528 | ACAACTTCGGTAGTTTTCAC | NC_045512.2 | 3′→5′ | 6461 | 6480 |
| 4529 | TACAACTTCGGTAGTTTTCA | NC_045512.2 | 3′→5′ | 6462 | 6481 |
| 4530 | CTACAACTTCGGTAGTTTTC | NC_045512.2 | 3′→5′ | 6463 | 6482 |
| 4531 | CCTACAACTTCGGTAGTTTT | NC_045512.2 | 3′→5′ | 6464 | 6483 |
| 4532 | CCATTAACAATAGTTGTACA | NC_045512.2 | 3′→5′ | 7532 | 7551 |
| 4533 | AAACCAACACTACCACATGA | NC_045512.2 | 3′→5′ | 10484 | 10503 |
| 4534 | AAAACCAACACTACCACATG | NC_045512.2 | 3′→5′ | 10485 | 10504 |
| 4535 | TAAAACCAACACTACCACAT | NC_045512.2 | 3′→5′ | 10486 | 10505 |
| 4536 | TTAAAACCAACACTACCACA | NC_045512.2 | 3′→5′ | 10487 | 10506 |
| 4537 | GTTAAAACCAACACTACCAC | NC_045512.2 | 3′→5′ | 10488 | 10507 |
| 4538 | TGTTAAAACCAACACTACCA | NC_045512.2 | 3′→5′ | 10489 | 10508 |
| 4539 | ATGTTAAAACCAACACTACC | NC_045512.2 | 3′→5′ | 10490 | 10509 |
| 4540 | TAGCCTAAGAAACAATAAAC | NC_045512.2 | 3′→5′ | 11609 | 11628 |
| 4541 | ATAGCCTAAGAAACAATAAA | NC_045512.2 | 3′→5′ | 11610 | 11629 |
| 4542 | AATAGCCTAAGAAACAATAA | NC_045512.2 | 3′→5′ | 11611 | 11630 |
| 4543 | GACATTTTAGACTGTACAGT | NC_045512.2 | 3′→5′ | 11834 | 11853 |
| 4544 | TCTACAGCACCCTGCATGGA | NC_045512.2 | 3′→5′ | 12023 | 12042 |
| 4545 | GTCTACAGCACCCTGCATGG | NC_045512.2 | 3′→5′ | 12024 | 12043 |
| 4546 | TGTCTACAGCACCCTGCATG | NC_045512.2 | 3′→5′ | 12025 | 12044 |
| 4547 | ATGTCTACAGCACCCTGCAT | NC_045512.2 | 3′→5′ | 12026 | 12045 |
| 4548 | GATTTAGCCACATTCAAAGA | NC_045512.2 | 3′→5′ | 12212 | 12231 |
| 4549 | AGATTTAGCCACATTCAAAG | NC_045512.2 | 3′→5′ | 12213 | 12232 |
| 4550 | CAGATTTAGCCACATTCAAA | NC_045512.2 | 3′→5′ | 12214 | 12233 |
| 4551 | TCAGATTTAGCCACATTCAA | NC_045512.2 | 3′→5′ | 12215 | 12234 |
| 4552 | GCATTGTTGATAATGTTGTT | NC_045512.2 | 3′→5′ | 12401 | 12420 |
| 4553 | TTAGGGAATCTAGCCCATTT | NC_045512.2 | 3′→5′ | 12839 | 12858 |
| 4554 | CTTAGGGAATCTAGCCCATT | NC_045512.2 | 3′→5′ | 12840 | 12859 |
| 4555 | TCTTAGGGAATCTAGCCCAT | NC_045512.2 | 3′→5′ | 12841 | 12860 |
| 4556 | CTCTTAGGGAATCTAGCCCA | NC_045512.2 | 3′→5′ | 12842 | 12861 |
| 4557 | ACTCTTAGGGAATCTAGCCC | NC_045512.2 | 3′→5′ | 12843 | 12862 |
| 4558 | CACTCTTAGGGAATCTAGCC | NC_045512.2 | 3′→5′ | 12844 | 12863 |
| 4559 | TCACTCTTAGGGAATCTAGC | NC_045512.2 | 3′→5′ | 12845 | 12864 |
| 4560 | ATCACTCTTAGGGAATCTAG | NC_045512.2 | 3′→5′ | 12846 | 12865 |
| 4561 | CATCACTCTTAGGGAATCTA | NC_045512.2 | 3′→5′ | 12847 | 12866 |
| 4562 | CCATCACTCTTAGGGAATCT | NC_045512.2 | 3′→5′ | 12848 | 12867 |
| 4563 | ACAAGGTGGTTCCAGTTCTG | NC_045512.2 | 3′→5′ | 12885 | 12904 |
| 4564 | TACAAGGTGGTTCCAGTTCT | NC_045512.2 | 3′→5′ | 12886 | 12905 |
| 4565 | CTACAAGGTGGTTCCAGTTC | NC_045512.2 | 3′→5′ | 12887 | 12906 |
| 4566 | CCTACAAGGTGGTTCCAGTT | NC_045512.2 | 3′→5′ | 12888 | 12907 |
| 4567 | ACCTACAAGGTGGTTCCAGT | NC_045512.2 | 3′→5′ | 12889 | 12908 |
| 4568 | AACCTACAAGGTGGTTCCAG | NC_045512.2 | 3′→5′ | 12890 | 12909 |
| 4569 | AAACCTACAAGGTGGTTCCA | NC_045512.2 | 3′→5′ | 12891 | 12910 |
| 4570 | CAAACCTACAAGGTGGTTCC | NC_045512.2 | 3′→5′ | 12892 | 12911 |
| 4571 | ACAAACCTACAAGGTGGTTC | NC_045512.2 | 3′→5′ | 12893 | 12912 |
| 4572 | AACAAACCTACAAGGTGGTT | NC_045512.2 | 3′→5′ | 12894 | 12913 |
| 4573 | TAACAAACCTACAAGGTGGT | NC_045512.2 | 3′→5′ | 12895 | 12914 |
| 4574 | GTAACAAACCTACAAGGTGG | NC_045512.2 | 3′→5′ | 12896 | 12915 |
| 4575 | TGTAACAAACCTACAAGGTG | NC_045512.2 | 3′→5′ | 12897 | 12916 |
| 4576 | CTGTAACAAACCTACAAGGT | NC_045512.2 | 3′→5′ | 12898 | 12917 |
| 4577 | TCTGTAACAAACCTACAAGG | NC_045512.2 | 3′→5′ | 12899 | 12918 |
| 4578 | GTCTGTAACAAACCTACAAG | NC_045512.2 | 3′→5′ | 12900 | 12919 |
| 4579 | TGTCTGTAACAAACCTACAA | NC_045512.2 | 3′→5′ | 12901 | 12920 |
| 4580 | GTGTCTGTAACAAACCTACA | NC_045512.2 | 3′→5′ | 12902 | 12921 |
| 4581 | TGTGTCTGTAACAAACCTAC | NC_045512.2 | 3′→5′ | 12903 | 12922 |
| 4582 | GTGTGTCTGTAACAAACCTA | NC_045512.2 | 3′→5′ | 12904 | 12923 |
| 4583 | GGTGTGTCTGTAACAAACCT | NC_045512.2 | 3′→5′ | 12905 | 12924 |
| 4584 | ACCTCTATTTAGGTTGTTTA | NC_045512.2 | 3′→5′ | 12966 | 12985 |
| 4585 | TACCTCTATTTAGGTTGTTT | NC_045512.2 | 3′→5′ | 12967 | 12986 |
| 4586 | ATACCTCTATTTAGGTTGTT | NC_045512.2 | 3′→5′ | 12968 | 12987 |
| 4587 | CATACCTCTATTTAGGTTGT | NC_045512.2 | 3′→5′ | 12969 | 12988 |
| 4588 | CCATACCTCTATTTAGGTTG | NC_045512.2 | 3′→5′ | 12970 | 12989 |
| 4589 | ACCATACCTCTATTTAGGTT | NC_045512.2 | 3′→5′ | 12971 | 12990 |
| 4590 | GTGTGTGTACACAACATCTT | NC_045512.2 | 3′→5′ | 13151 | 13170 |
| 4591 | AGTGTGTGTACACAACATCT | NC_045512.2 | 3′→5′ | 13152 | 13171 |
| 4592 | CAGTGTGTGTACACAACATC | NC_045512.2 | 3′→5′ | 13153 | 13172 |
| 4593 | CCAGTGTGTGTACACAACAT | NC_045512.2 | 3′→5′ | 13154 | 13173 |
| 4594 | ACCAGTGTGTGTACACAACA | NC_045512.2 | 3′→5′ | 13155 | 13174 |
| 4595 | TACCAGTGTGTGTACACAAC | NC_045512.2 | 3′→5′ | 13156 | 13175 |
| 4596 | GTACCAGTGTGTGTACACAA | NC_045512.2 | 3′→5′ | 13157 | 13176 |
| 4597 | AGACGGTACAGACTGTGTTT | NC_045512.2 | 3′→5′ | 13363 | 13382 |
| 4598 | CAGACGGTACAGACTGTGTT | NC_045512.2 | 3′→5′ | 13364 | 13383 |
| 4599 | GCAGACGGTACAGACTGTGT | NC_045512.2 | 3′→5′ | 13365 | 13384 |
| 4600 | CGCAGACGGTACAGACTGTG | NC_045512.2 | 3′→5′ | 13366 | 13385 |
| 4601 | CCGCAGACGGTACAGACTGT | NC_045512.2 | 3′→5′ | 13367 | 13386 |
| 4602 | CAGCCATAACCTTTCCACAT | NC_045512.2 | 3′→5′ | 13388 | 13407 |
| 4603 | ACAGCCATAACCTTTCCACA | NC_045512.2 | 3′→5′ | 13389 | 13408 |
| 4604 | TACAGCCATAACCTTTCCAC | NC_045512.2 | 3′→5′ | 13390 | 13409 |
| 4605 | CTACAGCCATAACCTTTCCA | NC_045512.2 | 3′→5′ | 13391 | 13410 |
| 4606 | ACTACAGCCATAACCTTTCC | NC_045512.2 | 3′→5′ | 13392 | 13411 |
| 4607 | AACTACAGCCATAACCTTTC | NC_045512.2 | 3′→5′ | 13393 | 13412 |
| 4608 | CAACTACAGCCATAACCTTT | NC_045512.2 | 3′→5′ | 13394 | 13413 |
| 4609 | ACAACTACAGCCATAACCTT | NC_045512.2 | 3′→5′ | 13395 | 13414 |
| 4610 | CACAACTACAGCCATAACCT | NC_045512.2 | 3′→5′ | 13396 | 13415 |
| 4611 | TCACAACTACAGCCATAACC | NC_045512.2 | 3′→5′ | 13397 | 13416 |
| 4612 | CGCAAACCCGTTTAAAAACG | NC_045512.2 | 3′→5′ | 13458 | 13477 |
| 4613 | CCGCAAACCCGTTTAAAAAC | NC_045512.2 | 3′→5′ | 13459 | 13478 |
| 4614 | ACCGCAAACCCGTTTAAAAA | NC_045512.2 | 3′→5′ | 13460 | 13479 |
| 4615 | CACCGCAAACCCGTTTAAAA | NC_045512.2 | 3′→5′ | 13461 | 13480 |
| 4616 | ACACCGCAAACCCGTTTAAA | NC_045512.2 | 3′→5′ | 13462 | 13481 |
| 4617 | TACACCGCAAACCCGTTTAA | NC_045512.2 | 3′→5′ | 13463 | 13482 |
| 4618 | TTACACCGCAAACCCGTTTA | NC_045512.2 | 3′→5′ | 13464 | 13483 |
| 4619 | CTTACACCGCAAACCCGTTT | NC_045512.2 | 3′→5′ | 13465 | 13484 |
| 4620 | ACTTACACCGCAAACCCGTT | NC_045512.2 | 3′→5′ | 13466 | 13485 |
| 4621 | CACTTACACCGCAAACCCGT | NC_045512.2 | 3′→5′ | 13467 | 13486 |
| 4622 | GCACTTACACCGCAAACCCG | NC_045512.2 | 3′→5′ | 13468 | 13487 |
| 4623 | TGCACTTACACCGCAAACCC | NC_045512.2 | 3′→5′ | 13469 | 13488 |
| 4624 | CTGCACTTACACCGCAAACC | NC_045512.2 | 3′→5′ | 13470 | 13489 |
| 4625 | GCTGCACTTACACCGCAAAC | NC_045512.2 | 3′→5′ | 13471 | 13490 |
| 4626 | GGCTGCACTTACACCGCAAA | NC_045512.2 | 3′→5′ | 13472 | 13491 |
| 4627 | GGGCTGCACTTACACCGCAA | NC_045512.2 | 3′→5′ | 13473 | 13492 |
| 4628 | CGGGCTGCACTTACACCGCA | NC_045512.2 | 3′→5′ | 13474 | 13493 |
| 4629 | ACGGGCTGCACTTACACCGC | NC_045512.2 | 3′→5′ | 13475 | 13494 |
| 4630 | GACGGGCTGCACTTACACCG | NC_045512.2 | 3′→5′ | 13476 | 13495 |
| 4631 | AGACGGGCTGCACTTACACC | NC_045512.2 | 3′→5′ | 13477 | 13496 |
| 4632 | AAGACGGGCTGCACTTACAC | NC_045512.2 | 3′→5′ | 13478 | 13497 |
| 4633 | TAAGACGGGCTGCACTTACA | NC_045512.2 | 3′→5′ | 13479 | 13498 |
| 4634 | GTAAGACGGGCTGCACTTAC | NC_045512.2 | 3′→5′ | 13480 | 13499 |
| 4635 | TGTAAGACGGGCTGCACTTA | NC_045512.2 | 3′→5′ | 13481 | 13500 |
| 4636 | GTGTAAGACGGGCTGCACTT | NC_045512.2 | 3′→5′ | 13482 | 13501 |
| 4637 | GGTGTAAGACGGGCTGCACT | NC_045512.2 | 3′→5′ | 13483 | 13502 |
| 4638 | CGGTGTAAGACGGGCTGCAC | NC_045512.2 | 3′→5′ | 13484 | 13503 |
| 4639 | ACGGTGTAAGACGGGCTGCA | NC_045512.2 | 3′→5′ | 13485 | 13504 |
| 4640 | CACGGTGTAAGACGGGCTGC | NC_045512.2 | 3′→5′ | 13486 | 13505 |
| 4641 | GCACGGTGTAAGACGGGCTG | NC_045512.2 | 3′→5′ | 13487 | 13506 |
| 4642 | CGCACGGTGTAAGACGGGCT | NC_045512.2 | 3′→5′ | 13488 | 13507 |
| 4643 | CCGCACGGTGTAAGACGGGC | NC_045512.2 | 3′→5′ | 13489 | 13508 |
| 4644 | GCCGCACGGTGTAAGACGGG | NC_045512.2 | 3′→5′ | 13490 | 13509 |
| 4645 | TGCCGCACGGTGTAAGACGG | NC_045512.2 | 3′→5′ | 13491 | 13510 |
| 4646 | GTGCCGCACGGTGTAAGACG | NC_045512.2 | 3′→5′ | 13492 | 13511 |
| 4647 | TGTGCCGCACGGTGTAAGAC | NC_045512.2 | 3′→5′ | 13493 | 13512 |
| 4648 | CTGTGCCGCACGGTGTAAGA | NC_045512.2 | 3′→5′ | 13494 | 13513 |
| 4649 | CCTGTGCCGCACGGTGTAAG | NC_045512.2 | 3′→5′ | 13495 | 13514 |
| 4650 | GCCTGTGCCGCACGGTGTAA | NC_045512.2 | 3′→5′ | 13496 | 13515 |
| 4651 | TGCCTGTGCCGCACGGTGTA | NC_045512.2 | 3′→5′ | 13497 | 13516 |
| 4652 | GTGCCTGTGCCGCACGGTGT | NC_045512.2 | 3′→5′ | 13498 | 13517 |
| 4653 | AGTGCCTGTGCCGCACGGTG | NC_045512.2 | 3′→5′ | 13499 | 13518 |
| 4654 | TAGTGCCTGTGCCGCACGGT | NC_045512.2 | 3′→5′ | 13500 | 13519 |
| 4655 | CTAGTGCCTGTGCCGCACGG | NC_045512.2 | 3′→5′ | 13501 | 13520 |
| 4656 | ATATGTGGTACCATGTCACC | NC_045512.2 | 3′→5′ | 13762 | 13781 |
| 4657 | TATATGTGGTACCATGTCAC | NC_045512.2 | 3′→5′ | 13763 | 13782 |
| 4658 | ATATATGTGGTACCATGTCA | NC_045512.2 | 3′→5′ | 13764 | 13783 |
| 4659 | GATATATGTGGTACCATGTC | NC_045512.2 | 3′→5′ | 13765 | 13784 |
| 4660 | TGATATATGTGGTACCATGT | NC_045512.2 | 3′→5′ | 13766 | 13785 |
| 4661 | GTGATATATGTGGTACCATG | NC_045512.2 | 3′→5′ | 13767 | 13786 |
| 4662 | CGTGATATATGTGGTACCAT | NC_045512.2 | 3′→5′ | 13768 | 13787 |
| 4663 | ACGTGATATATGTGGTACCA | NC_045512.2 | 3′→5′ | 13769 | 13788 |
| 4664 | GACGTGATATATGTGGTACC | NC_045512.2 | 3′→5′ | 13770 | 13789 |
| 4665 | TGACGTGATATATGTGGTAC | NC_045512.2 | 3′→5′ | 13771 | 13790 |
| 4666 | CAATATTTAAAATAACGGTC | NC_045512.2 | 3′→5′ | 14290 | 14309 |
| 4667 | CCAATATTTAAAATAACGGT | NC_045512.2 | 3′→5′ | 14291 | 14310 |
| 4668 | CCCAATATTTAAAATAACGG | NC_045512.2 | 3′→5′ | 14292 | 14311 |
| 4669 | TCCCAATATTTAAAATAACG | NC_045512.2 | 3′→5′ | 14293 | 14312 |
| 4670 | GGTCCAAAACTTGTAGGTGG | NC_045512.2 | 3′→5′ | 14404 | 14423 |
| 4671 | TGGTCCAAAACTTGTAGGTG | NC_045512.2 | 3′→5′ | 14405 | 14424 |
| 4672 | GTGGTCCAAAACTTGTAGGT | NC_045512.2 | 3′→5′ | 14406 | 14425 |
| 4673 | AGTGGTCCAAAACTTGTAGG | NC_045512.2 | 3′→5′ | 14407 | 14426 |
| 4674 | TAGTGGTCCAAAACTTGTAG | NC_045512.2 | 3′→5′ | 14408 | 14427 |
| 4675 | CTAGTGGTCCAAAACTTGTA | NC_045512.2 | 3′→5′ | 14409 | 14428 |
| 4676 | ACTAGTGGTCCAAAACTTGT | NC_045512.2 | 3′→5′ | 14410 | 14429 |
| 4677 | TTTACATCCTGATTATGTAC | NC_045512.2 | 3′→5′ | 14500 | 14519 |
| 4678 | GTTTACATCCTGATTATGTA | NC_045512.2 | 3′→5′ | 14501 | 14520 |
| 4679 | AGTTTACATCCTGATTATGT | NC_045512.2 | 3′→5′ | 14502 | 14521 |
| 4680 | AAGTTTACATCCTGATTATG | NC_045512.2 | 3′→5′ | 14503 | 14522 |
| 4681 | TAAGTTTACATCCTGATTAT | NC_045512.2 | 3′→5′ | 14504 | 14523 |
| 4682 | GTAAGTTTACATCCTGATTA | NC_045512.2 | 3′→5′ | 14505 | 14524 |
| 4683 | TGTAAGTTTACATCCTGATT | NC_045512.2 | 3′→5′ | 14506 | 14525 |
| 4684 | ATGTAAGTTTACATCCTGAT | NC_045512.2 | 3′→5′ | 14507 | 14526 |
| 4685 | TATGTAAGTTTACATCCTGA | NC_045512.2 | 3′→5′ | 14508 | 14527 |
| 4686 | CTATGTAAGTTTACATCCTG | NC_045512.2 | 3′→5′ | 14509 | 14528 |
| 4687 | GCTATGTAAGTTTACATCCT | NC_045512.2 | 3′→5′ | 14510 | 14529 |
| 4688 | AGCTATGTAAGTTTACATCC | NC_045512.2 | 3′→5′ | 14511 | 14530 |
| 4689 | GAGCTATGTAAGTTTACATC | NC_045512.2 | 3′→5′ | 14512 | 14531 |
| 4690 | AGTGCAGCTACTGAAAAGCA | NC_045512.2 | 3′→5′ | 14623 | 14642 |
| 4691 | ACAGTTTGAAAAGCAACATT | NC_045512.2 | 3′→5′ | 14650 | 14669 |
| 4692 | GACAGTTTGAAAAGCAACAT | NC_045512.2 | 3′→5′ | 14651 | 14670 |
| 4693 | TGACAGTTTGAAAAGCAACA | NC_045512.2 | 3′→5′ | 14652 | 14671 |
| 4694 | TTGACAGTTTGAAAAGCAAC | NC_045512.2 | 3′→5′ | 14653 | 14672 |
| 4695 | TTTGACAGTTTGAAAAGCAA | NC_045512.2 | 3′→5′ | 14654 | 14673 |
| 4696 | GTTTGACAGTTTGAAAAGCA | NC_045512.2 | 3′→5′ | 14655 | 14674 |
| 4697 | GGTTTGACAGTTTGAAAAGC | NC_045512.2 | 3′→5′ | 14656 | 14675 |
| 4698 | GGGTTTGACAGTTTGAAAAG | NC_045512.2 | 3′→5′ | 14657 | 14676 |
| 4699 | CGGGTTTGACAGTTTGAAAA | NC_045512.2 | 3′→5′ | 14658 | 14677 |
| 4700 | CCGGGTTTGACAGTTTGAAA | NC_045512.2 | 3′→5′ | 14659 | 14678 |
| 4701 | ACCGGGTTTGACAGTTTGAA | NC_045512.2 | 3′→5′ | 14660 | 14679 |
| 4702 | TACCGGGTTTGACAGTTTGA | NC_045512.2 | 3′→5′ | 14661 | 14680 |
| 4703 | TTACCGGGTTTGACAGTTTG | NC_045512.2 | 3′→5′ | 14662 | 14681 |
| 4704 | ATTACCGGGTTTGACAGTTT | NC_045512.2 | 3′→5′ | 14663 | 14682 |
| 4705 | AATTACCGGGTTTGACAGTT | NC_045512.2 | 3′→5′ | 14664 | 14683 |
| 4706 | AAATTACCGGGTTTGACAGT | NC_045512.2 | 3′→5′ | 14665 | 14684 |
| 4707 | AAAATTACCGGGTTTGACAG | NC_045512.2 | 3′→5′ | 14666 | 14685 |
| 4708 | TAAAATTACCGGGTTTGACA | NC_045512.2 | 3′→5′ | 14667 | 14686 |
| 4709 | TTAAAATTACCGGGTTTGAC | NC_045512.2 | 3′→5′ | 14668 | 14687 |
| 4710 | TTAGACACAGCAAAGTCATA | NC_045512.2 | 3′→5′ | 14698 | 14717 |
| 4711 | GAACTTCCTTCCTTAAAGAA | NC_045512.2 | 3′→5′ | 14722 | 14741 |
| 4712 | AGAACTTCCTTCCTTAAAGA | NC_045512.2 | 3′→5′ | 14723 | 14742 |
| 4713 | CAGAACTTCCTTCCTTAAAG | NC_045512.2 | 3′→5′ | 14724 | 14743 |
| 4714 | ACAGAACTTCCTTCCTTAAA | NC_045512.2 | 3′→5′ | 14725 | 14744 |
| 4715 | AACAGAACTTCCTTCCTTAA | NC_045512.2 | 3′→5′ | 14726 | 14745 |
| 4716 | CAACAGAACTTCCTTCCTTA | NC_045512.2 | 3′→5′ | 14727 | 14746 |
| 4717 | TCAACAGAACTTCCTTCCTT | NC_045512.2 | 3′→5′ | 14728 | 14747 |
| 4718 | TTCAACAGAACTTCCTTCCT | NC_045512.2 | 3′→5′ | 14729 | 14748 |
| 4719 | AGCAAAGAAGAAGTGTTTTA | NC_045512.2 | 3′→5′ | 14750 | 14769 |
| 4720 | GAGCAAAGAAGAAGTGTTTT | NC_045512.2 | 3′→5′ | 14751 | 14770 |
| 4721 | TGAGCAAAGAAGAAGTGTTT | NC_045512.2 | 3′→5′ | 14752 | 14771 |
| 4722 | CTGAGCAAAGAAGAAGTGTT | NC_045512.2 | 3′→5′ | 14753 | 14772 |
| 4723 | CCTGAGCAAAGAAGAAGTGT | NC_045512.2 | 3′→5′ | 14754 | 14773 |
| 4724 | TCCTGAGCAAAGAAGAAGTG | NC_045512.2 | 3′→5′ | 14755 | 14774 |
| 4725 | ATCCTGAGCAAAGAAGAAGT | NC_045512.2 | 3′→5′ | 14756 | 14775 |
| 4726 | CATCCTGAGCAAAGAAGAAG | NC_045512.2 | 3′→5′ | 14757 | 14776 |
| 4727 | CCATCCTGAGCAAAGAAGAA | NC_045512.2 | 3′→5′ | 14758 | 14777 |
| 4728 | CTGATATCACACATTGTTGG | NC_045512.2 | 3′→5′ | 14821 | 14840 |
| 4729 | TCTGATATCACACATTGTTG | NC_045512.2 | 3′→5′ | 14822 | 14841 |
| 4730 | GTCTGATATCACACATTGTT | NC_045512.2 | 3′→5′ | 14823 | 14842 |
| 4731 | TGTCTGATATCACACATTGT | NC_045512.2 | 3′→5′ | 14824 | 14843 |
| 4732 | TTGTCTGATATCACACATTG | NC_045512.2 | 3′→5′ | 14825 | 14844 |
| 4733 | GTTGTCTGATATCACACATT | NC_045512.2 | 3′→5′ | 14826 | 14845 |
| 4734 | AGTTGTCTGATATCACACAT | NC_045512.2 | 3′→5′ | 14827 | 14846 |
| 4735 | TTATCAACAACTTCAACTAC | NC_045512.2 | 3′→5′ | 14854 | 14873 |
| 4736 | CCATCGTAACAATCAAAGTA | NC_045512.2 | 3′→5′ | 14875 | 14894 |
| 4737 | ACCATCGTAACAATCAAAGT | NC_045512.2 | 3′→5′ | 14876 | 14895 |
| 4738 | CACCATCGTAACAATCAAAG | NC_045512.2 | 3′→5′ | 14877 | 14896 |
| 4739 | CCACCATCGTAACAATCAAA | NC_045512.2 | 3′→5′ | 14878 | 14897 |
| 4740 | GCCACCATCGTAACAATCAA | NC_045512.2 | 3′→5′ | 14879 | 14898 |
| 4741 | AGCCACCATCGTAACAATCA | NC_045512.2 | 3′→5′ | 14880 | 14899 |
| 4742 | CAGCCACCATCGTAACAATC | NC_045512.2 | 3′→5′ | 14881 | 14900 |
| 4743 | ACAGCCACCATCGTAACAAT | NC_045512.2 | 3′→5′ | 14882 | 14901 |
| 4744 | TACAGCCACCATCGTAACAA | NC_045512.2 | 3′→5′ | 14883 | 14902 |
| 4745 | ATACAGCCACCATCGTAACA | NC_045512.2 | 3′→5′ | 14884 | 14903 |
| 4746 | AGTCTAGCCTTACCCCATTT | NC_045512.2 | 3′→5′ | 14962 | 14981 |
| 4747 | AAGTCTAGCCTTACCCCATT | NC_045512.2 | 3′→5′ | 14963 | 14982 |
| 4748 | AAAGTCTAGCCTTACCCCAT | NC_045512.2 | 3′→5′ | 14964 | 14983 |
| 4749 | TAAAGTCTAGCCTTACCCCA | NC_045512.2 | 3′→5′ | 14965 | 14984 |
| 4750 | ATAAAGTCTAGCCTTACCCC | NC_045512.2 | 3′→5′ | 14966 | 14985 |
| 4751 | AATAAAGTCTAGCCTTACCC | NC_045512.2 | 3′→5′ | 14967 | 14986 |
| 4752 | TAATAAAGTCTAGCCTTACC | NC_045512.2 | 3′→5′ | 14968 | 14987 |
| 4753 | ATAATAAAGTCTAGCCTTAC | NC_045512.2 | 3′→5′ | 14969 | 14988 |
| 4754 | CATAATAAAGTCTAGCCTTA | NC_045512.2 | 3′→5′ | 14970 | 14989 |
| 4755 | TCATAATAAAGTCTAGCCTT | NC_045512.2 | 3′→5′ | 14971 | 14990 |
| 4756 | TGATCCTCATAACTCATTGA | NC_045512.2 | 3′→5′ | 14992 | 15011 |
| 4757 | TTGATCCTCATAACTCATTG | NC_045512.2 | 3′→5′ | 14993 | 15012 |
| 4758 | CTTGATCCTCATAACTCATT | NC_045512.2 | 3′→5′ | 14994 | 15013 |
| 4759 | TCTTGATCCTCATAACTCAT | NC_045512.2 | 3′→5′ | 14995 | 15014 |
| 4760 | ATCTTGATCCTCATAACTCA | NC_045512.2 | 3′→5′ | 14996 | 15015 |
| 4761 | CATCTTGATCCTCATAACTC | NC_045512.2 | 3′→5′ | 14997 | 15016 |
| 4762 | GCATCTTGATCCTCATAACT | NC_045512.2 | 3′→5′ | 14998 | 15017 |
| 4763 | TGCATCTTGATCCTCATAAC | NC_045512.2 | 3′→5′ | 14999 | 15018 |
| 4764 | GTGCATCTTGATCCTCATAA | NC_045512.2 | 3′→5′ | 15000 | 15019 |
| 4765 | AGTGCATCTTGATCCTCATA | NC_045512.2 | 3′→5′ | 15001 | 15020 |
| 4766 | TTAAGATTCATTTGAGTTAT | NC_045512.2 | 3′→5′ | 15055 | 15074 |
| 4767 | CTTAAGATTCATTTGAGTTA | NC_045512.2 | 3′→5′ | 15056 | 15075 |
| 4768 | ACTTAAGATTCATTTGAGTT | NC_045512.2 | 3′→5′ | 15057 | 15076 |
| 4769 | TACTTAAGATTCATTTGAGT | NC_045512.2 | 3′→5′ | 15058 | 15077 |
| 4770 | ATACTTAAGATTCATTTGAG | NC_045512.2 | 3′→5′ | 15059 | 15078 |
| 4771 | CATACTTAAGATTCATTTGA | NC_045512.2 | 3′→5′ | 15060 | 15079 |
| 4772 | GCATACTTAAGATTCATTTG | NC_045512.2 | 3′→5′ | 15061 | 15080 |
| 4773 | GGCATACTTAAGATTCATTT | NC_045512.2 | 3′→5′ | 15062 | 15081 |
| 4774 | TGGCATACTTAAGATTCATT | NC_045512.2 | 3′→5′ | 15063 | 15082 |
| 4775 | ATGGCATACTTAAGATTCAT | NC_045512.2 | 3′→5′ | 15064 | 15083 |
| 4776 | AATGGCATACTTAAGATTCA | NC_045512.2 | 3′→5′ | 15065 | 15084 |
| 4777 | TAATGGCATACTTAAGATTC | NC_045512.2 | 3′→5′ | 15066 | 15085 |
| 4778 | CTAATGGCATACTTAAGATT | NC_045512.2 | 3′→5′ | 15067 | 15086 |
| 4779 | ACTAATGGCATACTTAAGAT | NC_045512.2 | 3′→5′ | 15068 | 15087 |
| 4780 | CACTAATGGCATACTTAAGA | NC_045512.2 | 3′→5′ | 15069 | 15088 |
| 4781 | GCACTAATGGCATACTTAAG | NC_045512.2 | 3′→5′ | 15070 | 15089 |
| 4782 | TGCACTAATGGCATACTTAA | NC_045512.2 | 3′→5′ | 15071 | 15090 |
| 4783 | TTGCACTAATGGCATACTTA | NC_045512.2 | 3′→5′ | 15072 | 15091 |
| 4784 | TTTGCACTAATGGCATACTT | NC_045512.2 | 3′→5′ | 15073 | 15092 |
| 4785 | CTTTGCACTAATGGCATACT | NC_045512.2 | 3′→5′ | 15074 | 15093 |
| 4786 | TCTTTGCACTAATGGCATAC | NC_045512.2 | 3′→5′ | 15075 | 15094 |
| 4787 | TTCTTTGCACTAATGGCATA | NC_045512.2 | 3′→5′ | 15076 | 15095 |
| 4788 | ATTCTTTGCACTAATGGCAT | NC_045512.2 | 3′→5′ | 15077 | 15096 |
| 4789 | TATTCTTTGCACTAATGGCA | NC_045512.2 | 3′→5′ | 15078 | 15097 |
| 4790 | CTATTCTTTGCACTAATGGC | NC_045512.2 | 3′→5′ | 15079 | 15098 |
| 4791 | TCTATTCTTTGCACTAATGG | NC_045512.2 | 3′→5′ | 15080 | 15099 |
| 4792 | CTCTATTCTTTGCACTAATG | NC_045512.2 | 3′→5′ | 15081 | 15100 |
| 4793 | GCTCTATTCTTTGCACTAAT | NC_045512.2 | 3′→5′ | 15082 | 15101 |
| 4794 | AGCTCTATTCTTTGCACTAA | NC_045512.2 | 3′→5′ | 15083 | 15102 |
| 4795 | GAGCTCTATTCTTTGCACTA | NC_045512.2 | 3′→5′ | 15084 | 15103 |
| 4796 | CGAGCTCTATTCTTTGCACT | NC_045512.2 | 3′→5′ | 15085 | 15104 |
| 4797 | GCGAGCTCTATTCTTTGCAC | NC_045512.2 | 3′→5′ | 15086 | 15105 |
| 4798 | TGCGAGCTCTATTCTTTGCA | NC_045512.2 | 3′→5′ | 15087 | 15106 |
| 4799 | GTGCGAGCTCTATTCTTTGC | NC_045512.2 | 3′→5′ | 15088 | 15107 |
| 4800 | GGTGCGAGCTCTATTCTTTG | NC_045512.2 | 3′→5′ | 15089 | 15108 |
| 4801 | CGGTGCGAGCTCTATTCTTT | NC_045512.2 | 3′→5′ | 15090 | 15109 |
| 4802 | ACGGTGCGAGCTCTATTCTT | NC_045512.2 | 3′→5′ | 15091 | 15110 |
| 4803 | TACGGTGCGAGCTCTATTCT | NC_045512.2 | 3′→5′ | 15092 | 15111 |
| 4804 | CTACGGTGCGAGCTCTATTC | NC_045512.2 | 3′→5′ | 15093 | 15112 |
| 4805 | GCTACGGTGCGAGCTCTATT | NC_045512.2 | 3′→5′ | 15094 | 15113 |
| 4806 | AGCTACGGTGCGAGCTCTAT | NC_045512.2 | 3′→5′ | 15095 | 15114 |
| 4807 | CAGCTACGGTGCGAGCTCTA | NC_045512.2 | 3′→5′ | 15096 | 15115 |
| 4808 | CCAGCTACGGTGCGAGCTCT | NC_045512.2 | 3′→5′ | 15097 | 15116 |
| 4809 | ACCAGCTACGGTGCGAGCTC | NC_045512.2 | 3′→5′ | 15098 | 15117 |
| 4810 | CACCAGCTACGGTGCGAGCT | NC_045512.2 | 3′→5′ | 15099 | 15118 |
| 4811 | ACACCAGCTACGGTGCGAGC | NC_045512.2 | 3′→5′ | 15100 | 15119 |
| 4812 | GACACCAGCTACGGTGCGAG | NC_045512.2 | 3′→5′ | 15101 | 15120 |
| 4813 | AGACACCAGCTACGGTGCGA | NC_045512.2 | 3′→5′ | 15102 | 15121 |
| 4814 | GAGACACCAGCTACGGTGCG | NC_045512.2 | 3′→5′ | 15103 | 15122 |
| 4815 | AGAGACACCAGCTACGGTGC | NC_045512.2 | 3′→5′ | 15104 | 15123 |
| 4816 | TAGAGACACCAGCTACGGTG | NC_045512.2 | 3′→5′ | 15105 | 15124 |
| 4817 | ATAGAGACACCAGCTACGGT | NC_045512.2 | 3′→5′ | 15106 | 15125 |
| 4818 | GATAGAGACACCAGCTACGG | NC_045512.2 | 3′→5′ | 15107 | 15126 |
| 4819 | AGATAGAGACACCAGCTACG | NC_045512.2 | 3′→5′ | 15108 | 15127 |
| 4820 | CAGATAGAGACACCAGCTAC | NC_045512.2 | 3′→5′ | 15109 | 15128 |
| 4821 | ACAGATAGAGACACCAGCTA | NC_045512.2 | 3′→5′ | 15110 | 15129 |
| 4822 | TACAGATAGAGACACCAGCT | NC_045512.2 | 3′→5′ | 15111 | 15130 |
| 4823 | CTACAGATAGAGACACCAGC | NC_045512.2 | 3′→5′ | 15112 | 15131 |
| 4824 | ACTACAGATAGAGACACCAG | NC_045512.2 | 3′→5′ | 15113 | 15132 |
| 4825 | TACTACAGATAGAGACACCA | NC_045512.2 | 3′→5′ | 15114 | 15133 |
| 4826 | GTACTACAGATAGAGACACC | NC_045512.2 | 3′→5′ | 15115 | 15134 |
| 4827 | AGTACTACAGATAGAGACAC | NC_045512.2 | 3′→5′ | 15116 | 15135 |
| 4828 | TAGTACTACAGATAGAGACA | NC_045512.2 | 3′→5′ | 15117 | 15136 |
| 4829 | ATAGTACTACAGATAGAGAC | NC_045512.2 | 3′→5′ | 15118 | 15137 |
| 4830 | CATAGTACTACAGATAGAGA | NC_045512.2 | 3′→5′ | 15119 | 15138 |
| 4831 | TCATAGTACTACAGATAGAG | NC_045512.2 | 3′→5′ | 15120 | 15139 |
| 4832 | GTCATAGTACTACAGATAGA | NC_045512.2 | 3′→5′ | 15121 | 15140 |
| 4833 | CCTCTAGTGGCGGCTATTGA | NC_045512.2 | 3′→5′ | 15172 | 15191 |
| 4834 | TCCTCTAGTGGCGGCTATTG | NC_045512.2 | 3′→5′ | 15173 | 15192 |
| 4835 | CTCCTCTAGTGGCGGCTATT | NC_045512.2 | 3′→5′ | 15174 | 15193 |
| 4836 | GCTCCTCTAGTGGCGGCTAT | NC_045512.2 | 3′→5′ | 15175 | 15194 |
| 4837 | AGCTCCTCTAGTGGCGGCTA | NC_045512.2 | 3′→5′ | 15176 | 15195 |
| 4838 | TAGCTCCTCTAGTGGCGGCT | NC_045512.2 | 3′→5′ | 15177 | 15196 |
| 4839 | GTAGCTCCTCTAGTGGCGGC | NC_045512.2 | 3′→5′ | 15178 | 15197 |
| 4840 | AGTAGCTCCTCTAGTGGCGG | NC_045512.2 | 3′→5′ | 15179 | 15198 |
| 4841 | CAGTAGCTCCTCTAGTGGCG | NC_045512.2 | 3′→5′ | 15180 | 15199 |
| 4842 | ACAGTAGCTCCTCTAGTGGC | NC_045512.2 | 3′→5′ | 15181 | 15200 |
| 4843 | AGCATGTTAGGCATGGCTCT | NC_045512.2 | 3′→5′ | 15310 | 15329 |
| 4844 | AAGCATGTTAGGCATGGCTC | NC_045512.2 | 3′→5′ | 15311 | 15330 |
| 4845 | TAAGCATGTTAGGCATGGCT | NC_045512.2 | 3′→5′ | 15312 | 15331 |
| 4846 | CTAAGCATGTTAGGCATGGC | NC_045512.2 | 3′→5′ | 15313 | 15332 |
| 4847 | TGTTTGCGAGCAAGAACAAG | NC_045512.2 | 3′→5′ | 15346 | 15365 |
| 4848 | ATGTTTGCGAGCAAGAACAA | NC_045512.2 | 3′→5′ | 15347 | 15366 |
| 4849 | TATGTTTGCGAGCAAGAACA | NC_045512.2 | 3′→5′ | 15348 | 15367 |
| 4850 | CTATTAGCATAAGCAGTTGT | NC_045512.2 | 3′→5′ | 15496 | 15515 |
| 4851 | ACTATTAGCATAAGCAGTTG | NC_045512.2 | 3′→5′ | 15497 | 15516 |
| 4852 | CACTATTAGCATAAGCAGTT | NC_045512.2 | 3′→5′ | 15498 | 15517 |
| 4853 | ACACTATTAGCATAAGCAGT | NC_045512.2 | 3′→5′ | 15499 | 15518 |
| 4854 | TTTCTATAGAGACACTCATA | NC_045512.2 | 3′→5′ | 15622 | 15641 |
| 4855 | ATTTCTATAGAGACACTCAT | NC_045512.2 | 3′→5′ | 15623 | 15642 |
| 4856 | TATTTCTATAGAGACACTCA | NC_045512.2 | 3′→5′ | 15624 | 15643 |
| 4857 | CTATTTCTATAGAGACACTC | NC_045512.2 | 3′→5′ | 15625 | 15644 |
| 4858 | GTAAGGTCAGTCTCAGTCCA | NC_045512.2 | 3′→5′ | 15838 | 15857 |
| 4859 | AGTAAGGTCAGTCTCAGTCC | NC_045512.2 | 3′→5′ | 15839 | 15858 |
| 4860 | TAGTAAGGTCAGTCTCAGTC | NC_045512.2 | 3′→5′ | 15840 | 15859 |
| 4861 | TTAGTAAGGTCAGTCTCAGT | NC_045512.2 | 3′→5′ | 15841 | 15860 |
| 4862 | TTTAGTAAGGTCAGTCTCAG | NC_045512.2 | 3′→5′ | 15842 | 15861 |
| 4863 | CTTTAGTAAGGTCAGTCTCA | NC_045512.2 | 3′→5′ | 15843 | 15862 |
| 4864 | CCTTTAGTAAGGTCAGTCTC | NC_045512.2 | 3′→5′ | 15844 | 15863 |
| 4865 | TCCTTTAGTAAGGTCAGTCT | NC_045512.2 | 3′→5′ | 15845 | 15864 |
| 4866 | GTCCTTTAGTAAGGTCAGTC | NC_045512.2 | 3′→5′ | 15846 | 15865 |
| 4867 | GGTCCTTTAGTAAGGTCAGT | NC_045512.2 | 3′→5′ | 15847 | 15866 |
| 4868 | AGGTCCTTTAGTAAGGTCAG | NC_045512.2 | 3′→5′ | 15848 | 15867 |
| 4869 | GAGGTCCTTTAGTAAGGTCA | NC_045512.2 | 3′→5′ | 15849 | 15868 |
| 4870 | TGAGGTCCTTTAGTAAGGTC | NC_045512.2 | 3′→5′ | 15850 | 15869 |
| 4871 | TGTTTAACTAGCATTGTATG | NC_045512.2 | 3′→5′ | 15886 | 15905 |
| 4872 | ATAAGTGTACCATCTGTTTT | NC_045512.2 | 3′→5′ | 15985 | 16004 |
| 4873 | CATAAGTGTACCATCTGTTT | NC_045512.2 | 3′→5′ | 15986 | 16005 |
| 4874 | TCATAAGTGTACCATCTGTT | NC_045512.2 | 3′→5′ | 15987 | 16006 |
| 4875 | ATCATAAGTGTACCATCTGT | NC_045512.2 | 3′→5′ | 15988 | 16007 |
| 4876 | AATCATAAGTGTACCATCTG | NC_045512.2 | 3′→5′ | 15989 | 16008 |
| 4877 | CAATCATAAGTGTACCATCT | NC_045512.2 | 3′→5′ | 15990 | 16009 |
| 4878 | TCAATCATAAGTGTACCATC | NC_045512.2 | 3′→5′ | 15991 | 16010 |
| 4879 | TCAGCATACTCCTGATTAGG | NC_045512.2 | 3′→5′ | 16057 | 16076 |
| 4880 | ATCAGCATACTCCTGATTAG | NC_045512.2 | 3′→5′ | 16058 | 16077 |
| 4881 | CATCAGCATACTCCTGATTA | NC_045512.2 | 3′→5′ | 16059 | 16078 |
| 4882 | ACATCAGCATACTCCTGATT | NC_045512.2 | 3′→5′ | 16060 | 16079 |
| 4883 | TCATAAAACTCAGGTTCCCA | NC_045512.2 | 3′→5′ | 16186 | 16205 |
| 4884 | TTTTCAAAGGTGTACTCTCC | NC_045512.2 | 3′→5′ | 16822 | 16841 |
| 4885 | TTTTTCAAAGGTGTACTCTC | NC_045512.2 | 3′→5′ | 16823 | 16842 |
| 4886 | CTTTTTCAAAGGTGTACTCT | NC_045512.2 | 3′→5′ | 16824 | 16843 |
| 4887 | CCTTTTTCAAAGGTGTACTC | NC_045512.2 | 3′→5′ | 16825 | 16844 |
| 4888 | ACCTTTTTCAAAGGTGTACT | NC_045512.2 | 3′→5′ | 16826 | 16845 |
| 4889 | CACCTTTTTCAAAGGTGTAC | NC_045512.2 | 3′→5′ | 16827 | 16846 |
| 4890 | TCACCTTTTTCAAAGGTGTA | NC_045512.2 | 3′→5′ | 16828 | 16847 |
| 4891 | GTCACCTTTTTCAAAGGTGT | NC_045512.2 | 3′→5′ | 16829 | 16848 |
| 4892 | AGTCACCTTTTTCAAAGGTG | NC_045512.2 | 3′→5′ | 16830 | 16849 |
| 4893 | TAGTCACCTTTTTCAAAGGT | NC_045512.2 | 3′→5′ | 16831 | 16850 |
| 4894 | ATAGTCACCTTTTTCAAAGG | NC_045512.2 | 3′→5′ | 16832 | 16851 |
| 4895 | CATAGTCACCTTTTTCAAAG | NC_045512.2 | 3′→5′ | 16833 | 16852 |
| 4896 | CCATAGTCACCTTTTTCAAA | NC_045512.2 | 3′→5′ | 16834 | 16853 |
| 4897 | ACCATAGTCACCTTTTTCAA | NC_045512.2 | 3′→5′ | 16835 | 16854 |
| 4898 | CACCATAGTCACCTTTTTCA | NC_045512.2 | 3′→5′ | 16836 | 16855 |
| 4899 | TCACCATAGTCACCTTTTTC | NC_045512.2 | 3′→5′ | 16837 | 16856 |
| 4900 | ATCACCATAGTCACCTTTTT | NC_045512.2 | 3′→5′ | 16838 | 16857 |
| 4901 | CATCACCATAGTCACCTTTT | NC_045512.2 | 3′→5′ | 16839 | 16858 |
| 4902 | GCATCACCATAGTCACCTTT | NC_045512.2 | 3′→5′ | 16840 | 16859 |
| 4903 | AGCATCACCATAGTCACCTT | NC_045512.2 | 3′→5′ | 16841 | 16860 |
| 4904 | CAGCATCACCATAGTCACCT | NC_045512.2 | 3′→5′ | 16842 | 16861 |
| 4905 | ACAGCATCACCATAGTCACC | NC_045512.2 | 3′→5′ | 16843 | 16862 |
| 4906 | AACAGCATCACCATAGTCAC | NC_045512.2 | 3′→5′ | 16844 | 16863 |
| 4907 | CAACAGCATCACCATAGTCA | NC_045512.2 | 3′→5′ | 16845 | 16864 |
| 4908 | ACAACAGCATCACCATAGTC | NC_045512.2 | 3′→5′ | 16846 | 16865 |
| 4909 | TAGTGCTCTTGTGGCACTAG | NC_045512.2 | 3′→5′ | 16954 | 16973 |
| 4910 | ATAGTGCTCTTGTGGCACTA | NC_045512.2 | 3′→5′ | 16955 | 16974 |
| 4911 | CATAGTGCTCTTGTGGCACT | NC_045512.2 | 3′→5′ | 16956 | 16975 |
| 4912 | ACATAGTGCTCTTGTGGCAC | NC_045512.2 | 3′→5′ | 16957 | 16976 |
| 4913 | CTAGAAAACTCATCTGAGAT | NC_045512.2 | 3′→5′ | 17008 | 17027 |
| 4914 | GCTAGAAAACTCATCTGAGA | NC_045512.2 | 3′→5′ | 17009 | 17028 |
| 4915 | TGCTAGAAAACTCATCTGAG | NC_045512.2 | 3′→5′ | 17010 | 17029 |
| 4916 | TTGCTAGAAAACTCATCTGA | NC_045512.2 | 3′→5′ | 17011 | 17030 |
| 4917 | ATTGCTAGAAAACTCATCTG | NC_045512.2 | 3′→5′ | 17012 | 17031 |
| 4918 | CATTGCTAGAAAACTCATCT | NC_045512.2 | 3′→5′ | 17013 | 17032 |
| 4919 | ACATTGCTAGAAAACTCATC | NC_045512.2 | 3′→5′ | 17014 | 17033 |
| 4920 | AACATTGCTAGAAAACTCAT | NC_045512.2 | 3′→5′ | 17015 | 17034 |
| 4921 | CAACATTGCTAGAAAACTCA | NC_045512.2 | 3′→5′ | 17016 | 17035 |
| 4922 | GCAACATTGCTAGAAAACTC | NC_045512.2 | 3′→5′ | 17017 | 17036 |
| 4923 | TGCAACATTGCTAGAAAACT | NC_045512.2 | 3′→5′ | 17018 | 17037 |
| 4924 | TTGCAACATTGCTAGAAAAC | NC_045512.2 | 3′→5′ | 17019 | 17038 |
| 4925 | TTTGCAACATTGCTAGAAAA | NC_045512.2 | 3′→5′ | 17020 | 17039 |
| 4926 | ATTTGCAACATTGCTAGAAA | NC_045512.2 | 3′→5′ | 17021 | 17040 |
| 4927 | AATTTGCAACATTGCTAGAA | NC_045512.2 | 3′→5′ | 17022 | 17041 |
| 4928 | TAATTTGCAACATTGCTAGA | NC_045512.2 | 3′→5′ | 17023 | 17042 |
| 4929 | TTACCAGTACCAGGTGGTCC | NC_045512.2 | 3′→5′ | 17080 | 17099 |
| 4930 | CTTACCAGTACCAGGTGGTC | NC_045512.2 | 3′→5′ | 17081 | 17100 |
| 4931 | TCTTACCAGTACCAGGTGGT | NC_045512.2 | 3′→5′ | 17082 | 17101 |
| 4932 | CTCTTACCAGTACCAGGTGG | NC_045512.2 | 3′→5′ | 17083 | 17102 |
| 4933 | ACTCTTACCAGTACCAGGTG | NC_045512.2 | 3′→5′ | 17084 | 17103 |
| 4934 | GACTCTTACCAGTACCAGGT | NC_045512.2 | 3′→5′ | 17085 | 17104 |
| 4935 | TGACTCTTACCAGTACCAGG | NC_045512.2 | 3′→5′ | 17086 | 17105 |
| 4936 | ATGACTCTTACCAGTACCAG | NC_045512.2 | 3′→5′ | 17087 | 17106 |
| 4937 | AATGACTCTTACCAGTACCA | NC_045512.2 | 3′→5′ | 17088 | 17107 |
| 4938 | AAATGACTCTTACCAGTACC | NC_045512.2 | 3′→5′ | 17089 | 17108 |
| 4939 | AAAATGACTCTTACCAGTAC | NC_045512.2 | 3′→5′ | 17090 | 17109 |
| 4940 | CAAAATGACTCTTACCAGTA | NC_045512.2 | 3′→5′ | 17091 | 17110 |
| 4941 | GCAAAATGACTCTTACCAGT | NC_045512.2 | 3′→5′ | 17092 | 17111 |
| 4942 | GTATACACTATGCGAGCAGA | NC_045512.2 | 3′→5′ | 17137 | 17156 |
| 4943 | GTTGAATTCACTTTGAATTT | NC_045512.2 | 3′→5′ | 17269 | 17288 |
| 4944 | TGTTGAATTCACTTTGAATT | NC_045512.2 | 3′→5′ | 17270 | 17289 |
| 4945 | AGGAACATGTCTGGACCTAT | NC_045512.2 | 3′→5′ | 17530 | 17549 |
| 4946 | ACAATTTCAGCAGGACAACG | NC_045512.2 | 3′→5′ | 17563 | 17582 |
| 4947 | ATTGCAGATGAAACATCATG | NC_045512.2 | 3′→5′ | 17680 | 17699 |
| 4948 | ACAGCTTTTCTCCAAGCAGG | NC_045512.2 | 3′→5′ | 17746 | 17765 |
| 4949 | GTGAATATGACATAGTCATA | NC_045512.2 | 3′→5′ | 17857 | 17876 |
| 4950 | TCTCTATCAGACATTATGCA | NC_045512.2 | 3′→5′ | 17956 | 17975 |
| 4951 | AGGTGTGTAGGTGCCTGTGT | NC_045512.2 | 3′→5′ | 18100 | 18119 |
| 4952 | GAGGTGTGTAGGTGCCTGTG | NC_045512.2 | 3′→5′ | 18101 | 18120 |
| 4953 | TGAGGTGTGTAGGTGCCTGT | NC_045512.2 | 3′→5′ | 18102 | 18121 |
| 4954 | CTGAGGTGTGTAGGTGCCTG | NC_045512.2 | 3′→5′ | 18103 | 18122 |
| 4955 | CCCATCATAGAGATGAGTCT | NC_045512.2 | 3′→5′ | 18196 | 18215 |
| 4956 | ACCCATCATAGAGATGAGTC | NC_045512.2 | 3′→5′ | 18197 | 18216 |
| 4957 | AACCCATCATAGAGATGAGT | NC_045512.2 | 3′→5′ | 18198 | 18217 |
| 4958 | AAACCCATCATAGAGATGAG | NC_045512.2 | 3′→5′ | 18199 | 18218 |
| 4959 | GCCACATTTTCTAAACTCTG | NC_045512.2 | 3′→5′ | 19618 | 19637 |
| 4960 | AGCCACATTTTCTAAACTCT | NC_045512.2 | 3′→5′ | 19619 | 19638 |
| 4961 | AAGCCACATTTTCTAAACTC | NC_045512.2 | 3′→5′ | 19620 | 19639 |
| 4962 | CGCTTAGCCCAAAGCTCAAA | NC_045512.2 | 3′→5′ | 19783 | 19802 |
| 4963 | ACACCCAAATTATTGAGTAT | NC_045512.2 | 3′→5′ | 19831 | 19850 |
| 4964 | CCAATTAATGTGACTCCATT | NC_045512.2 | 3′→5′ | 20107 | 20126 |
| 4965 | TCCAATTAATGTGACTCCAT | NC_045512.2 | 3′→5′ | 20108 | 20127 |
| 4966 | CTCCAATTAATGTGACTCCA | NC_045512.2 | 3′→5′ | 20109 | 20128 |
| 4967 | TCTCCAATTAATGTGACTCC | NC_045512.2 | 3′→5′ | 20110 | 20129 |
| 4968 | TTCTCCAATTAATGTGACTC | NC_045512.2 | 3′→5′ | 20111 | 20130 |
| 4969 | TTTGCGACATTCATCATTAT | NC_045512.2 | 3′→5′ | 20776 | 20795 |
| 4970 | ACTCTGTTGTTTTCTCTAAT | NC_045512.2 | 3′→5′ | 21502 | 21521 |
| 4971 | AACTCTGTTGTTTTCTCTAA | NC_045512.2 | 3′→5′ | 21503 | 21522 |
| 4972 | CAACTCTGTTGTTTTCTCTA | NC_045512.2 | 3′→5′ | 21504 | 21523 |
| 4973 | ACAACTCTGTTGTTTTCTCT | NC_045512.2 | 3′→5′ | 21505 | 21524 |
| 4974 | TGGTTCTCATAGAGAACATT | NC_045512.2 | 3′→5′ | 24302 | 24321 |
| 4975 | TTGGTTCTCATAGAGAACAT | NC_045512.2 | 3′→5′ | 24303 | 24322 |
| 4976 | TTTGGTTCTCATAGAGAACA | NC_045512.2 | 3′→5′ | 24304 | 24323 |
| 4977 | TTTTGGTTCTCATAGAGAAC | NC_045512.2 | 3′→5′ | 24305 | 24324 |
| 4978 | TTTTTGGTTCTCATAGAGAA | NC_045512.2 | 3′→5′ | 24306 | 24325 |
| 4979 | GAGCTAAGTTGTTTAACAAG | NC_045512.2 | 3′→5′ | 24446 | 24465 |
| 4980 | GCAGCAAGATTAGCAGAAGC | NC_045512.2 | 3′→5′ | 24620 | 24639 |
| 4981 | AGCAGCAAGATTAGCAGAAG | NC_045512.2 | 3′→5′ | 24621 | 24640 |
| 4982 | TAGCAGCAAGATTAGCAGAA | NC_045512.2 | 3′→5′ | 24622 | 24641 |
| 4983 | GTAGCAGCAAGATTAGCAGA | NC_045512.2 | 3′→5′ | 24623 | 24642 |
| 4984 | AGTAGCAGCAAGATTAGCAG | NC_045512.2 | 3′→5′ | 24624 | 24643 |
| 4985 | TAGTAGCAGCAAGATTAGCA | NC_045512.2 | 3′→5′ | 24625 | 24644 |
| 4986 | TTAGTAGCAGCAAGATTAGC | NC_045512.2 | 3′→5′ | 24626 | 24645 |
| 4987 | TTTAGTAGCAGCAAGATTAG | NC_045512.2 | 3′→5′ | 24627 | 24646 |
| 4988 | TTTTAGTAGCAGCAAGATTA | NC_045512.2 | 3′→5′ | 24628 | 24647 |
| 4989 | ATTTTAGTAGCAGCAAGATT | NC_045512.2 | 3′→5′ | 24629 | 24648 |
| 4990 | CATTTTAGTAGCAGCAAGAT | NC_045512.2 | 3′→5′ | 24630 | 24649 |
| 4991 | ACATTTTAGTAGCAGCAAGA | NC_045512.2 | 3′→5′ | 24631 | 24650 |
| 4992 | GACATTTTAGTAGCAGCAAG | NC_045512.2 | 3′→5′ | 24632 | 24651 |
| 4993 | ACTCTTTTTGATTGTCCAAG | NC_045512.2 | 3′→5′ | 24662 | 24681 |
| 4994 | AACTCTTTTTGATTGTCCAA | NC_045512.2 | 3′→5′ | 24663 | 24682 |
| 4995 | CAACTCTTTTTGATTGTCCA | NC_045512.2 | 3′→5′ | 24664 | 24683 |
| 4996 | TCAACTCTTTTTGATTGTCC | NC_045512.2 | 3′→5′ | 24665 | 24684 |
| 4997 | ACATCTGGTGATGTATGATT | NC_045512.2 | 3′→5′ | 25034 | 25053 |
| 4998 | AACATCTGGTGATGTATGAT | NC_045512.2 | 3′→5′ | 25035 | 25054 |
| 4999 | CAACATCTGGTGATGTATGA | NC_045512.2 | 3′→5′ | 25036 | 25055 |
| 5000 | TCAACATCTGGTGATGTATG | NC_045512.2 | 3′→5′ | 25037 | 25056 |
| 5001 | ATCAACATCTGGTGATGTAT | NC_045512.2 | 3′→5′ | 25038 | 25057 |
| 5002 | ATTGAGGCGGTCAATTTCTT | NC_045512.2 | 3′→5′ | 25104 | 25123 |
| 5003 | CATTGAGGCGGTCAATTTCT | NC_045512.2 | 3′→5′ | 25105 | 25124 |
| 5004 | TCATTGAGGCGGTCAATTTC | NC_045512.2 | 3′→5′ | 25106 | 25125 |
| 5005 | CTCATTGAGGCGGTCAATTT | NC_045512.2 | 3′→5′ | 25107 | 25126 |
| 5006 | CCTCATTGAGGCGGTCAATT | NC_045512.2 | 3′→5′ | 25108 | 25127 |
| 5007 | ACCTCATTGAGGCGGTCAAT | NC_045512.2 | 3′→5′ | 25109 | 25128 |
| 5008 | TATGTGTAATGTAATTTGAC | NC_045512.2 | 3′→5′ | 25364 | 25383 |
| 5009 | TTATGTGTAATGTAATTTGA | NC_045512.2 | 3′→5′ | 25365 | 25384 |
| 5010 | TTTATGTGTAATGTAATTTG | NC_045512.2 | 3′→5′ | 25366 | 25385 |
| 5011 | GTTTATGTGTAATGTAATTT | NC_045512.2 | 3′→5′ | 25367 | 25386 |
| 5012 | CGTTTATGTGTAATGTAATT | NC_045512.2 | 3′→5′ | 25368 | 25387 |
| 5013 | GAAAGGGAGTGAGGCTTGTA | NC_045512.2 | 3′→5′ | 25502 | 25521 |
| 5014 | CGAAAGGGAGTGAGGCTTGT | NC_045512.2 | 3′→5′ | 25503 | 25522 |
| 5015 | CCGAAAGGGAGTGAGGCTTG | NC_045512.2 | 3′→5′ | 25504 | 25523 |
| 5016 | TCCGAAAGGGAGTGAGGCTT | NC_045512.2 | 3′→5′ | 25505 | 25524 |
| 5017 | ATCCGAAAGGGAGTGAGGCT | NC_045512.2 | 3′→5′ | 25506 | 25525 |
| 5018 | CATCCGAAAGGGAGTGAGGC | NC_045512.2 | 3′→5′ | 25507 | 25526 |
| 5019 | CCATCCGAAAGGGAGTGAGG | NC_045512.2 | 3′→5′ | 25508 | 25527 |
| 5020 | GCCATCCGAAAGGGAGTGAG | NC_045512.2 | 3′→5′ | 25509 | 25528 |
| 5021 | AGCCATCCGAAAGGGAGTGA | NC_045512.2 | 3′→5′ | 25510 | 25529 |
| 5022 | AAGCCATCCGAAAGGGAGTG | NC_045512.2 | 3′→5′ | 25511 | 25530 |
| 5023 | ACGCTAGTAGTCGTCGTCGG | NC_045512.2 | 3′→5′ | 26191 | 26210 |
| 5024 | CACGCTAGTAGTCGTCGTCG | NC_045512.2 | 3′→5′ | 26192 | 26211 |
| 5025 | GCACGCTAGTAGTCGTCGTC | NC_045512.2 | 3′→5′ | 26193 | 26212 |
| 5026 | GGCACGCTAGTAGTCGTCGT | NC_045512.2 | 3′→5′ | 26194 | 26213 |
| 5027 | AGGCACGCTAGTAGTCGTCG | NC_045512.2 | 3′→5′ | 26195 | 26214 |
| 5028 | AAGGCACGCTAGTAGTCGTC | NC_045512.2 | 3′→5′ | 26196 | 26215 |
| 5029 | AAAGGCACGCTAGTAGTCGT | NC_045512.2 | 3′→5′ | 26197 | 26216 |
| 5030 | CAAAGGCACGCTAGTAGTCG | NC_045512.2 | 3′→5′ | 26198 | 26217 |
| 5031 | ACAAAGGCACGCTAGTAGTC | NC_045512.2 | 3′→5′ | 26199 | 26218 |
| 5032 | TACAAAGGCACGCTAGTAGT | NC_045512.2 | 3′→5′ | 26200 | 26219 |
| 5033 | TTACAAAGGCACGCTAGTAG | NC_045512.2 | 3′→5′ | 26201 | 26220 |
| 5034 | CTTACAAAGGCACGCTAGTA | NC_045512.2 | 3′→5′ | 26202 | 26221 |
| 5035 | GCTTACAAAGGCACGCTAGT | NC_045512.2 | 3′→5′ | 26203 | 26222 |
| 5036 | TGCTTACAAAGGCACGCTAG | NC_045512.2 | 3′→5′ | 26204 | 26223 |
| 5037 | GTGCTTACAAAGGCACGCTA | NC_045512.2 | 3′→5′ | 26205 | 26224 |
| 5038 | TGTGCTTACAAAGGCACGCT | NC_045512.2 | 3′→5′ | 26206 | 26225 |
| 5039 | TTGTGCTTACAAAGGCACGC | NC_045512.2 | 3′→5′ | 26207 | 26226 |
| 5040 | CTTGTGCTTACAAAGGCACG | NC_045512.2 | 3′→5′ | 26208 | 26227 |
| 5041 | AGTACATAAGTTCGTACTCA | NC_045512.2 | 3′→5′ | 26232 | 26251 |
| 5042 | GAGTACATAAGTTCGTACTC | NC_045512.2 | 3′→5′ | 26233 | 26252 |
| 5043 | TGAGTACATAAGTTCGTACT | NC_045512.2 | 3′→5′ | 26234 | 26253 |
| 5044 | ATGAGTACATAAGTTCGTAC | NC_045512.2 | 3′→5′ | 26235 | 26254 |
| 5045 | AATGAGTACATAAGTTCGTA | NC_045512.2 | 3′→5′ | 26236 | 26255 |
| 5046 | GAATGAGTACATAAGTTCGT | NC_045512.2 | 3′→5′ | 26237 | 26256 |
| 5047 | CGAATGAGTACATAAGTTCG | NC_045512.2 | 3′→5′ | 26238 | 26257 |
| 5048 | ACGAATGAGTACATAAGTTC | NC_045512.2 | 3′→5′ | 26239 | 26258 |
| 5049 | AACGAATGAGTACATAAGTT | NC_045512.2 | 3′→5′ | 26240 | 26259 |
| 5050 | AAACGAATGAGTACATAAGT | NC_045512.2 | 3′→5′ | 26241 | 26260 |
| 5051 | GAAACGAATGAGTACATAAG | NC_045512.2 | 3′→5′ | 26242 | 26261 |
| 5052 | CGAAACGAATGAGTACATAA | NC_045512.2 | 3′→5′ | 26243 | 26262 |
| 5053 | CCGAAACGAATGAGTACATA | NC_045512.2 | 3′→5′ | 26244 | 26263 |
| 5054 | TCCGAAACGAATGAGTACAT | NC_045512.2 | 3′→5′ | 26245 | 26264 |
| 5055 | TTCCGAAACGAATGAGTACA | NC_045512.2 | 3′→5′ | 26246 | 26265 |
| 5056 | CTTCCGAAACGAATGAGTAC | NC_045512.2 | 3′→5′ | 26247 | 26266 |
| 5057 | TCTTCCGAAACGAATGAGTA | NC_045512.2 | 3′→5′ | 26248 | 26267 |
| 5058 | TTAACTATTAACGTACCTGT | NC_045512.2 | 3′→5′ | 26269 | 26288 |
| 5059 | ATTAACTATTAACGTACCTG | NC_045512.2 | 3′→5′ | 26270 | 26289 |
| 5060 | TATTAACTATTAACGTACCT | NC_045512.2 | 3′→5′ | 26271 | 26290 |
| 5061 | CTATTAACTATTAACGTACC | NC_045512.2 | 3′→5′ | 26272 | 26291 |
| 5062 | GCTATTAACTATTAACGTAC | NC_045512.2 | 3′→5′ | 26273 | 26292 |
| 5063 | CGCTATTAACTATTAACGTA | NC_045512.2 | 3′→5′ | 26274 | 26293 |
| 5064 | ACGCTATTAACTATTAACGT | NC_045512.2 | 3′→5′ | 26275 | 26294 |
| 5065 | TACGCTATTAACTATTAACG | NC_045512.2 | 3′→5′ | 26276 | 26295 |
| 5066 | GTACGCTATTAACTATTAAC | NC_045512.2 | 3′→5′ | 26277 | 26296 |
| 5067 | AGTACGCTATTAACTATTAA | NC_045512.2 | 3′→5′ | 26278 | 26297 |
| 5068 | AAGTACGCTATTAACTATTA | NC_045512.2 | 3′→5′ | 26279 | 26298 |
| 5069 | GAAGTACGCTATTAACTATT | NC_045512.2 | 3′→5′ | 26280 | 26299 |
| 5070 | AGAAGTACGCTATTAACTAT | NC_045512.2 | 3′→5′ | 26281 | 26300 |
| 5071 | AAGAAGTACGCTATTAACTA | NC_045512.2 | 3′→5′ | 26282 | 26301 |
| 5072 | AAAGAAGTACGCTATTAACT | NC_045512.2 | 3′→5′ | 26283 | 26302 |
| 5073 | AAAAGAAGTACGCTATTAAC | NC_045512.2 | 3′→5′ | 26284 | 26303 |
| 5074 | AAAAAGAAGTACGCTATTAA | NC_045512.2 | 3′→5′ | 26285 | 26304 |
| 5075 | GAAAAAGAAGTACGCTATTA | NC_045512.2 | 3′→5′ | 26286 | 26305 |
| 5076 | AGAAAAAGAAGTACGCTATT | NC_045512.2 | 3′→5′ | 26287 | 26306 |
| 5077 | AAGAAAAAGAAGTACGCTAT | NC_045512.2 | 3′→5′ | 26288 | 26307 |
| 5078 | CAAGAAAAAGAAGTACGCTA | NC_045512.2 | 3′→5′ | 26289 | 26308 |
| 5079 | GCAAGAAAAAGAAGTACGCT | NC_045512.2 | 3′→5′ | 26290 | 26309 |
| 5080 | AGCAAGAAAAAGAAGTACGC | NC_045512.2 | 3′→5′ | 26291 | 26310 |
| 5081 | AAGCAAGAAAAAGAAGTACG | NC_045512.2 | 3′→5′ | 26292 | 26311 |
| 5082 | AAAGCAAGAAAAAGAAGTAC | NC_045512.2 | 3′→5′ | 26293 | 26312 |
| 5083 | GAAAGCAAGAAAAAGAAGTA | NC_045512.2 | 3′→5′ | 26294 | 26313 |
| 5084 | CGAAAGCAAGAAAAAGAAGT | NC_045512.2 | 3′→5′ | 26295 | 26314 |
| 5085 | ACGAAAGCAAGAAAAAGAAG | NC_045512.2 | 3′→5′ | 26296 | 26315 |
| 5086 | CACGAAAGCAAGAAAAAGAA | NC_045512.2 | 3′→5′ | 26297 | 26316 |
| 5087 | CCACGAAAGCAAGAAAAAGA | NC_045512.2 | 3′→5′ | 26298 | 26317 |
| 5088 | ACCACGAAAGCAAGAAAAAG | NC_045512.2 | 3′→5′ | 26299 | 26318 |
| 5089 | TACCACGAAAGCAAGAAAAA | NC_045512.2 | 3′→5′ | 26300 | 26319 |
| 5090 | ATACCACGAAAGCAAGAAAA | NC_045512.2 | 3′→5′ | 26301 | 26320 |
| 5091 | AATACCACGAAAGCAAGAAA | NC_045512.2 | 3′→5′ | 26302 | 26321 |
| 5092 | GAATACCACGAAAGCAAGAA | NC_045512.2 | 3′→5′ | 26303 | 26322 |
| 5093 | AGAATACCACGAAAGCAAGA | NC_045512.2 | 3′→5′ | 26304 | 26323 |
| 5094 | AAGAATACCACGAAAGCAAG | NC_045512.2 | 3′→5′ | 26305 | 26324 |
| 5095 | CAAGAATACCACGAAAGCAA | NC_045512.2 | 3′→5′ | 26306 | 26325 |
| 5096 | GCAAGAATACCACGAAAGCA | NC_045512.2 | 3′→5′ | 26307 | 26326 |
| 5097 | AGCAAGAATACCACGAAAGC | NC_045512.2 | 3′→5′ | 26308 | 26327 |
| 5098 | TAGCAAGAATACCACGAAAG | NC_045512.2 | 3′→5′ | 26309 | 26328 |
| 5099 | CTAGCAAGAATACCACGAAA | NC_045512.2 | 3′→5′ | 26310 | 26329 |
| 5100 | ACTAGCAAGAATACCACGAA | NC_045512.2 | 3′→5′ | 26311 | 26330 |
| 5101 | GCAGTAAGGATGGCTAGTGT | NC_045512.2 | 3′→5′ | 26332 | 26351 |
| 5102 | CGCAGTAAGGATGGCTAGTG | NC_045512.2 | 3′→5′ | 26333 | 26352 |
| 5103 | GCGCAGTAAGGATGGCTAGT | NC_045512.2 | 3′→5′ | 26334 | 26353 |
| 5104 | AGCGCAGTAAGGATGGCTAG | NC_045512.2 | 3′→5′ | 26335 | 26354 |
| 5105 | AAGCGCAGTAAGGATGGCTA | NC_045512.2 | 3′→5′ | 26336 | 26355 |
| 5106 | GAAGCGCAGTAAGGATGGCT | NC_045512.2 | 3′→5′ | 26337 | 26356 |
| 5107 | CGAAGCGCAGTAAGGATGGC | NC_045512.2 | 3′→5′ | 26338 | 26357 |
| 5108 | TCGAAGCGCAGTAAGGATGG | NC_045512.2 | 3′→5′ | 26339 | 26358 |
| 5109 | ATCGAAGCGCAGTAAGGATG | NC_045512.2 | 3′→5′ | 26340 | 26359 |
| 5110 | AATCGAAGCGCAGTAAGGAT | NC_045512.2 | 3′→5′ | 26341 | 26360 |
| 5111 | CAATCGAAGCGCAGTAAGGA | NC_045512.2 | 3′→5′ | 26342 | 26361 |
| 5112 | ACAATCGAAGCGCAGTAAGG | NC_045512.2 | 3′→5′ | 26343 | 26362 |
| 5113 | CACAATCGAAGCGCAGTAAG | NC_045512.2 | 3′→5′ | 26344 | 26363 |
| 5114 | ACACAATCGAAGCGCAGTAA | NC_045512.2 | 3′→5′ | 26345 | 26364 |
| 5115 | CACACAATCGAAGCGCAGTA | NC_045512.2 | 3′→5′ | 26346 | 26365 |
| 5116 | GCACACAATCGAAGCGCAGT | NC_045512.2 | 3′→5′ | 26347 | 26366 |
| 5117 | CGCACACAATCGAAGCGCAG | NC_045512.2 | 3′→5′ | 26348 | 26367 |
| 5118 | ACGCACACAATCGAAGCGCA | NC_045512.2 | 3′→5′ | 26349 | 26368 |
| 5119 | TACGCACACAATCGAAGCGC | NC_045512.2 | 3′→5′ | 26350 | 26369 |
| 5120 | GTACGCACACAATCGAAGCG | NC_045512.2 | 3′→5′ | 26351 | 26370 |
| 5121 | AGTACGCACACAATCGAAGC | NC_045512.2 | 3′→5′ | 26352 | 26371 |
| 5122 | CAGTACGCACACAATCGAAG | NC_045512.2 | 3′→5′ | 26353 | 26372 |
| 5123 | GCAGTACGCACACAATCGAA | NC_045512.2 | 3′→5′ | 26354 | 26373 |
| 5124 | AGCAGTACGCACACAATCGA | NC_045512.2 | 3′→5′ | 26355 | 26374 |
| 5125 | CAGCAGTACGCACACAATCG | NC_045512.2 | 3′→5′ | 26356 | 26375 |
| 5126 | GCAGCAGTACGCACACAATC | NC_045512.2 | 3′→5′ | 26357 | 26376 |
| 5127 | TGCAGCAGTACGCACACAAT | NC_045512.2 | 3′→5′ | 26358 | 26377 |
| 5128 | TTGCAGCAGTACGCACACAA | NC_045512.2 | 3′→5′ | 26359 | 26378 |
| 5129 | ATTGCAGCAGTACGCACACA | NC_045512.2 | 3′→5′ | 26360 | 26379 |
| 5130 | TATTGCAGCAGTACGCACAC | NC_045512.2 | 3′→5′ | 26361 | 26380 |
| 5131 | ATATTGCAGCAGTACGCACA | NC_045512.2 | 3′→5′ | 26362 | 26381 |
| 5132 | AATATTGCAGCAGTACGCAC | NC_045512.2 | 3′→5′ | 26363 | 26382 |
| 5133 | CAATATTGCAGCAGTACGCA | NC_045512.2 | 3′→5′ | 26364 | 26383 |
| 5134 | ACAATATTGCAGCAGTACGC | NC_045512.2 | 3′→5′ | 26365 | 26384 |
| 5135 | AACAATATTGCAGCAGTACG | NC_045512.2 | 3′→5′ | 26366 | 26385 |
| 5136 | TAACAATATTGCAGCAGTAC | NC_045512.2 | 3′→5′ | 26367 | 26386 |
| 5137 | TTAACAATATTGCAGCAGTA | NC_045512.2 | 3′→5′ | 26368 | 26387 |
| 5138 | GTTAACAATATTGCAGCAGT | NC_045512.2 | 3′→5′ | 26369 | 26388 |
| 5139 | CGTTAACAATATTGCAGCAG | NC_045512.2 | 3′→5′ | 26370 | 26389 |
| 5140 | ACGTTAACAATATTGCAGCA | NC_045512.2 | 3′→5′ | 26371 | 26390 |
| 5141 | CACGTTAACAATATTGCAGC | NC_045512.2 | 3′→5′ | 26372 | 26391 |
| 5142 | TCACGTTAACAATATTGCAG | NC_045512.2 | 3′→5′ | 26373 | 26392 |
| 5143 | CTCACGTTAACAATATTGCA | NC_045512.2 | 3′→5′ | 26374 | 26393 |
| 5144 | ACTCACGTTAACAATATTGC | NC_045512.2 | 3′→5′ | 26375 | 26394 |
| 5145 | GACCAGAAGATCAGGAACTC | NC_045512.2 | 3′→5′ | 26450 | 26469 |
| 5146 | AGACCAGAAGATCAGGAACT | NC_045512.2 | 3′→5′ | 26451 | 26470 |
| 5147 | TAGACCAGAAGATCAGGAAC | NC_045512.2 | 3′→5′ | 26452 | 26471 |
| 5148 | TTAGACCAGAAGATCAGGAA | NC_045512.2 | 3′→5′ | 26453 | 26472 |
| 5149 | TTTAGACCAGAAGATCAGGA | NC_045512.2 | 3′→5′ | 26454 | 26473 |
| 5150 | GTTTAGACCAGAAGATCAGG | NC_045512.2 | 3′→5′ | 26455 | 26474 |
| 5151 | CGTTTAGACCAGAAGATCAG | NC_045512.2 | 3′→5′ | 26456 | 26475 |
| 5152 | TCGTTTAGACCAGAAGATCA | NC_045512.2 | 3′→5′ | 26457 | 26476 |
| 5153 | TTCGTTTAGACCAGAAGATC | NC_045512.2 | 3′→5′ | 26458 | 26477 |
| 5154 | GTTCGTTTAGACCAGAAGAT | NC_045512.2 | 3′→5′ | 26459 | 26478 |
| 5155 | AGTTCGTTTAGACCAGAAGA | NC_045512.2 | 3′→5′ | 26460 | 26479 |
| 5156 | TAGTTCGTTTAGACCAGAAG | NC_045512.2 | 3′→5′ | 26461 | 26480 |
| 5157 | TTAGTTCGTTTAGACCAGAA | NC_045512.2 | 3′→5′ | 26462 | 26481 |
| 5158 | ATTACTAGGTTCCATTGTTC | NC_045512.2 | 3′→5′ | 26574 | 26593 |
| 5159 | TATTACTAGGTTCCATTGTT | NC_045512.2 | 3′→5′ | 26575 | 26594 |
| 5160 | CTATTACTAGGTTCCATTGT | NC_045512.2 | 3′→5′ | 26576 | 26595 |
| 5161 | CCTATTACTAGGTTCCATTG | NC_045512.2 | 3′→5′ | 26577 | 26596 |
| 5162 | ACCTATTACTAGGTTCCATT | NC_045512.2 | 3′→5′ | 26578 | 26597 |
| 5163 | AACCTATTACTAGGTTCCAT | NC_045512.2 | 3′→5′ | 26579 | 26598 |
| 5164 | AAACCTATTACTAGGTTCCA | NC_045512.2 | 3′→5′ | 26580 | 26599 |
| 5165 | GAAACCTATTACTAGGTTCC | NC_045512.2 | 3′→5′ | 26581 | 26600 |
| 5166 | GAAAGCGTTCGTGATGTAGC | NC_045512.2 | 3′→5′ | 27033 | 27052 |
| 5167 | AGAAAGCGTTCGTGATGTAG | NC_045512.2 | 3′→5′ | 27034 | 27053 |
| 5168 | AAGAAAGCGTTCGTGATGTA | NC_045512.2 | 3′→5′ | 27035 | 27054 |
| 5169 | TAAGAAAGCGTTCGTGATGT | NC_045512.2 | 3′→5′ | 27036 | 27055 |
| 5170 | ATAAGAAAGCGTTCGTGATG | NC_045512.2 | 3′→5′ | 27037 | 27056 |
| 5171 | AATAAGAAAGCGTTCGTGAT | NC_045512.2 | 3′→5′ | 27038 | 27057 |
| 5172 | TAATAAGAAAGCGTTCGTGA | NC_045512.2 | 3′→5′ | 27039 | 27058 |
| 5173 | GTAATAAGAAAGCGTTCGTG | NC_045512.2 | 3′→5′ | 27040 | 27059 |
| 5174 | TGTAATAAGAAAGCGTTCGT | NC_045512.2 | 3′→5′ | 27041 | 27060 |
| 5175 | TTGTAATAAGAAAGCGTTCG | NC_045512.2 | 3′→5′ | 27042 | 27061 |
| 5176 | TTTGTAATAAGAAAGCGTTC | NC_045512.2 | 3′→5′ | 27043 | 27062 |
| 5177 | ATTTGTAATAAGAAAGCGTT | NC_045512.2 | 3′→5′ | 27044 | 27063 |
| 5178 | AATTTGTAATAAGAAAGCGT | NC_045512.2 | 3′→5′ | 27045 | 27064 |
| 5179 | TCTGTTGTCACTTACTGTAC | NC_045512.2 | 3′→5′ | 27183 | 27202 |
| 5180 | ATCTGTTGTCACTTACTGTA | NC_045512.2 | 3′→5′ | 27184 | 27203 |
| 5181 | CATCTGTTGTCACTTACTGT | NC_045512.2 | 3′→5′ | 27185 | 27204 |
| 5182 | ACATCTGTTGTCACTTACTG | NC_045512.2 | 3′→5′ | 27186 | 27205 |
| 5183 | AACATCTGTTGTCACTTACT | NC_045512.2 | 3′→5′ | 27187 | 27206 |
| 5184 | AAACATCTGTTGTCACTTAC | NC_045512.2 | 3′→5′ | 27188 | 27207 |
| 5185 | GAAACATCTGTTGTCACTTA | NC_045512.2 | 3′→5′ | 27189 | 27208 |
| 5186 | TGAAACATCTGTTGTCACTT | NC_045512.2 | 3′→5′ | 27190 | 27209 |
| 5187 | ATGAAACATCTGTTGTCACT | NC_045512.2 | 3′→5′ | 27191 | 27210 |
| 5188 | GATGAAACATCTGTTGTCAC | NC_045512.2 | 3′→5′ | 27192 | 27211 |
| 5189 | AGATGAAACATCTGTTGTCA | NC_045512.2 | 3′→5′ | 27193 | 27212 |
| 5190 | ATTTTCATGTTCGTTTAATC | NC_045512.2 | 3′→5′ | 27382 | 27401 |
| 5191 | AATTTTCATGTTCGTTTAAT | NC_045512.2 | 3′→5′ | 27383 | 27402 |
| 5192 | TAATTTTCATGTTCGTTTAA | NC_045512.2 | 3′→5′ | 27384 | 27403 |
| 5193 | ATAATTTTCATGTTCGTTTA | NC_045512.2 | 3′→5′ | 27385 | 27404 |
| 5194 | AATAATTTTCATGTTCGTTT | NC_045512.2 | 3′→5′ | 27386 | 27405 |
| 5195 | GAATAATTTTCATGTTCGTT | NC_045512.2 | 3′→5′ | 27387 | 27406 |
| 5196 | AGAATAATTTTCATGTTCGT | NC_045512.2 | 3′→5′ | 27388 | 27407 |
| 5197 | AATGGTGAATTGCCCTCGTA | NC_045512.2 | 3′→5′ | 27511 | 27530 |
| 5198 | AAATGGTGAATTGCCCTCGT | NC_045512.2 | 3′→5′ | 27512 | 27531 |
| 5199 | GAAATGGTGAATTGCCCTCG | NC_045512.2 | 3′→5′ | 27513 | 27532 |
| 5200 | TGAAATGGTGAATTGCCCTC | NC_045512.2 | 3′→5′ | 27514 | 27533 |
| 5201 | CACAAATAGAAGTCAATTAA | NC_045512.2 | 3′→5′ | 27771 | 27790 |
| 5202 | GCACAAATAGAAGTCAATTA | NC_045512.2 | 3′→5′ | 27772 | 27791 |
| 5203 | AGCACAAATAGAAGTCAATT | NC_045512.2 | 3′→5′ | 27773 | 27792 |
| 5204 | AAGCACAAATAGAAGTCAAT | NC_045512.2 | 3′→5′ | 27774 | 27793 |
| 5205 | AAAGCACAAATAGAAGTCAA | NC_045512.2 | 3′→5′ | 27775 | 27794 |
| 5206 | AAAAGCACAAATAGAAGTCA | NC_045512.2 | 3′→5′ | 27776 | 27795 |
| 5207 | AAAAAGCACAAATAGAAGTC | NC_045512.2 | 3′→5′ | 27777 | 27796 |
| 5208 | TAAAAAGCACAAATAGAAGT | NC_045512.2 | 3′→5′ | 27778 | 27797 |
| 5209 | CTAAAAAGCACAAATAGAAG | NC_045512.2 | 3′→5′ | 27779 | 27798 |
| 5210 | GCTAAAAAGCACAAATAGAA | NC_045512.2 | 3′→5′ | 27780 | 27799 |
| 5211 | GGCTAAAAAGCACAAATAGA | NC_045512.2 | 3′→5′ | 27781 | 27800 |
| 5212 | AGGCTAAAAAGCACAAATAG | NC_045512.2 | 3′→5′ | 27782 | 27801 |
| 5213 | AAGGCTAAAAAGCACAAATA | NC_045512.2 | 3′→5′ | 27783 | 27802 |
| 5214 | AAAGGCTAAAAAGCACAAAT | NC_045512.2 | 3′→5′ | 27784 | 27803 |
| 5215 | GAAAGGCTAAAAAGCACAAA | NC_045512.2 | 3′→5′ | 27785 | 27804 |
| 5216 | AGAAAGGCTAAAAAGCACAA | NC_045512.2 | 3′→5′ | 27786 | 27805 |
| 5217 | CAGAAAGGCTAAAAAGCACA | NC_045512.2 | 3′→5′ | 27787 | 27806 |
| 5218 | GCAGAAAGGCTAAAAAGCAC | NC_045512.2 | 3′→5′ | 27788 | 27807 |
| 5219 | AGCAGAAAGGCTAAAAAGCA | NC_045512.2 | 3′→5′ | 27789 | 27808 |
| 5220 | TAGCAGAAAGGCTAAAAAGC | NC_045512.2 | 3′→5′ | 27790 | 27809 |
| 5221 | ATAGCAGAAAGGCTAAAAAG | NC_045512.2 | 3′→5′ | 27791 | 27810 |
| 5222 | AATAGCAGAAAGGCTAAAAA | NC_045512.2 | 3′→5′ | 27792 | 27811 |
| 5223 | GAATAGCAGAAAGGCTAAAA | NC_045512.2 | 3′→5′ | 27793 | 27812 |
| 5224 | GGAATAGCAGAAAGGCTAAA | NC_045512.2 | 3′→5′ | 27794 | 27813 |
| 5225 | AGGAATAGCAGAAAGGCTAA | NC_045512.2 | 3′→5′ | 27795 | 27814 |
| 5226 | AAGGAATAGCAGAAAGGCTA | NC_045512.2 | 3′→5′ | 27796 | 27815 |
| 5227 | CAAGGAATAGCAGAAAGGCT | NC_045512.2 | 3′→5′ | 27797 | 27816 |
| 5228 | ACAAGGAATAGCAGAAAGGC | NC_045512.2 | 3′→5′ | 27798 | 27817 |
| 5229 | AACAAGGAATAGCAGAAAGG | NC_045512.2 | 3′→5′ | 27799 | 27818 |
| 5230 | GTCCATTATCAGACATTTTA | NC_045512.2 | 3′→5′ | 28270 | 28289 |
| 5231 | GGTCCATTATCAGACATTTT | NC_045512.2 | 3′→5′ | 28271 | 28290 |
| 5232 | GGGTCCATTATCAGACATTT | NC_045512.2 | 3′→5′ | 28272 | 28291 |
| 5233 | GGGGTCCATTATCAGACATT | NC_045512.2 | 3′→5′ | 28273 | 28292 |
| 5234 | TGGGGTCCATTATCAGACAT | NC_045512.2 | 3′→5′ | 28274 | 28293 |
| 5235 | TTGGGGTCCATTATCAGACA | NC_045512.2 | 3′→5′ | 28275 | 28294 |
| 5236 | TTTGGGGTCCATTATCAGAC | NC_045512.2 | 3′→5′ | 28276 | 28295 |
| 5237 | TTTTGGGGTCCATTATCAGA | NC_045512.2 | 3′→5′ | 28277 | 28296 |
| 5238 | TTATTGGGTAAACCTTGGGG | NC_045512.2 | 3′→5′ | 28397 | 28416 |
| 5239 | ATTATTGGGTAAACCTTGGG | NC_045512.2 | 3′→5′ | 28398 | 28417 |
| 5240 | TATTATTGGGTAAACCTTGG | NC_045512.2 | 3′→5′ | 28399 | 28418 |
| 5241 | GTATTATTGGGTAAACCTTG | NC_045512.2 | 3′→5′ | 28400 | 28419 |
| 5242 | AGTATTATTGGGTAAACCTT | NC_045512.2 | 3′→5′ | 28401 | 28420 |
| 5243 | CAGTATTATTGGGTAAACCT | NC_045512.2 | 3′→5′ | 28402 | 28421 |
| 5244 | GCAGTATTATTGGGTAAACC | NC_045512.2 | 3′→5′ | 28403 | 28422 |
| 5245 | CGCAGTATTATTGGGTAAAC | NC_045512.2 | 3′→5′ | 28404 | 28423 |
| 5246 | ACGCAGTATTATTGGGTAAA | NC_045512.2 | 3′→5′ | 28405 | 28424 |
| 5247 | GACGCAGTATTATTGGGTAA | NC_045512.2 | 3′→5′ | 28406 | 28425 |
| 5248 | AGACGCAGTATTATTGGGTA | NC_045512.2 | 3′→5′ | 28407 | 28426 |
| 5249 | AAGACGCAGTATTATTGGGT | NC_045512.2 | 3′→5′ | 28408 | 28427 |
| 5250 | CAAGACGCAGTATTATTGGG | NC_045512.2 | 3′→5′ | 28409 | 28428 |
| 5251 | CCAAGACGCAGTATTATTGG | NC_045512.2 | 3′→5′ | 28410 | 28429 |
| 5252 | ACCAAGACGCAGTATTATTG | NC_045512.2 | 3′→5′ | 28411 | 28430 |
| 5253 | AACCAAGACGCAGTATTATT | NC_045512.2 | 3′→5′ | 28412 | 28431 |
| 5254 | GAACCAAGACGCAGTATTAT | NC_045512.2 | 3′→5′ | 28413 | 28432 |
| 5255 | TGAACCAAGACGCAGTATTA | NC_045512.2 | 3′→5′ | 28414 | 28433 |
| 5256 | GTGAACCAAGACGCAGTATT | NC_045512.2 | 3′→5′ | 28415 | 28434 |
| 5257 | AGTAGCCAATTTGGTCATCT | NC_045512.2 | 3′→5′ | 28513 | 28532 |
| 5258 | TAGTAGCCAATTTGGTCATC | NC_045512.2 | 3′→5′ | 28514 | 28533 |
| 5259 | GTAGTAGCCAATTTGGTCAT | NC_045512.2 | 3′→5′ | 28515 | 28534 |
| 5260 | GGTAGTAGCCAATTTGGTCA | NC_045512.2 | 3′→5′ | 28516 | 28535 |
| 5261 | CGGTAGTAGCCAATTTGGTC | NC_045512.2 | 3′→5′ | 28517 | 28536 |
| 5262 | TCGGTAGTAGCCAATTTGGT | NC_045512.2 | 3′→5′ | 28518 | 28537 |
| 5263 | TTCGGTAGTAGCCAATTTGG | NC_045512.2 | 3′→5′ | 28519 | 28538 |
| 5264 | CTTCGGTAGTAGCCAATTTG | NC_045512.2 | 3′→5′ | 28520 | 28539 |
| 5265 | TCTTCGGTAGTAGCCAATTT | NC_045512.2 | 3′→5′ | 28521 | 28540 |
| 5266 | CTCTTCGGTAGTAGCCAATT | NC_045512.2 | 3′→5′ | 28522 | 28541 |
| 5267 | GCTCTTCGGTAGTAGCCAAT | NC_045512.2 | 3′→5′ | 28523 | 28542 |
| 5268 | AGCTCTTCGGTAGTAGCCAA | NC_045512.2 | 3′→5′ | 28524 | 28543 |
| 5269 | TAGCTCTTCGGTAGTAGCCA | NC_045512.2 | 3′→5′ | 28525 | 28544 |
| 5270 | GTAGCTCTTCGGTAGTAGCC | NC_045512.2 | 3′→5′ | 28526 | 28545 |
| 5271 | GGTAGCTCTTCGGTAGTAGC | NC_045512.2 | 3′→5′ | 28527 | 28546 |
| 5272 | TTCAAGGCTCCCTCAGTTGC | NC_045512.2 | 3′→5′ | 28673 | 28692 |
| 5273 | GGATTGCGGGTGCCAATGTG | NC_045512.2 | 3′→5′ | 28706 | 28725 |
| 5274 | AGGATTGCGGGTGCCAATGT | NC_045512.2 | 3′→5′ | 28707 | 28726 |
| 5275 | CTTGAGGAAGTTGTAGCACG | NC_045512.2 | 3′→5′ | 28744 | 28763 |
| 5276 | CCTTGAGGAAGTTGTAGCAC | NC_045512.2 | 3′→5′ | 28745 | 28764 |
| 5277 | TCCTTGAGGAAGTTGTAGCA | NC_045512.2 | 3′→5′ | 28746 | 28765 |
| 5278 | TTCCTTGAGGAAGTTGTAGC | NC_045512.2 | 3′→5′ | 28747 | 28766 |
| 5279 | GTTCCTTGAGGAAGTTGTAG | NC_045512.2 | 3′→5′ | 28748 | 28767 |
| 5280 | TGTTCCTTGAGGAAGTTGTA | NC_045512.2 | 3′→5′ | 28749 | 28768 |
| 5281 | TTGTTCCTTGAGGAAGTTGT | NC_045512.2 | 3′→5′ | 28750 | 28769 |
| 5282 | GTTGTTCCTTGAGGAAGTTG | NC_045512.2 | 3′→5′ | 28751 | 28770 |
| 5283 | TGTTGTTCCTTGAGGAAGTT | NC_045512.2 | 3′→5′ | 28752 | 28771 |
| 5284 | ATGTTGTTCCTTGAGGAAGT | NC_045512.2 | 3′→5′ | 28753 | 28772 |
| 5285 | AATGTTGTTCCTTGAGGAAG | NC_045512.2 | 3′→5′ | 28754 | 28773 |
| 5286 | CAATGTTGTTCCTTGAGGAA | NC_045512.2 | 3′→5′ | 28755 | 28774 |
| 5287 | GCAATGTTGTTCCTTGAGGA | NC_045512.2 | 3′→5′ | 28756 | 28775 |
| 5288 | GGCAATGTTGTTCCTTGAGG | NC_045512.2 | 3′→5′ | 28757 | 28776 |
| 5289 | TGGCAATGTTGTTCCTTGAG | NC_045512.2 | 3′→5′ | 28758 | 28777 |
| 5290 | TTGGCAATGTTGTTCCTTGA | NC_045512.2 | 3′→5′ | 28759 | 28778 |
| 5291 | TTTGGCAATGTTGTTCCTTG | NC_045512.2 | 3′→5′ | 28760 | 28779 |
| 5292 | TTTTGGCAATGTTGTTCCTT | NC_045512.2 | 3′→5′ | 28761 | 28780 |
| 5293 | CTTTTGGCAATGTTGTTCCT | NC_045512.2 | 3′→5′ | 28762 | 28781 |
| 5294 | CCTTTTGGCAATGTTGTTCC | NC_045512.2 | 3′→5′ | 28763 | 28782 |
| 5295 | GCCTTTTGGCAATGTTGTTC | NC_045512.2 | 3′→5′ | 28764 | 28783 |
| 5296 | AGCCTTTTGGCAATGTTGTT | NC_045512.2 | 3′→5′ | 28765 | 28784 |
| 5297 | AAGCCTTTTGGCAATGTTGT | NC_045512.2 | 3′→5′ | 28766 | 28785 |
| 5298 | GAAGCCTTTTGGCAATGTTG | NC_045512.2 | 3′→5′ | 28767 | 28786 |
| 5299 | AGAAGCCTTTTGGCAATGTT | NC_045512.2 | 3′→5′ | 28768 | 28787 |
| 5300 | TAGAAGCCTTTTGGCAATGT | NC_045512.2 | 3′→5′ | 28769 | 28788 |
| 5301 | GTAGAAGCCTTTTGGCAATG | NC_045512.2 | 3′→5′ | 28770 | 28789 |
| 5302 | CGTAGAAGCCTTTTGGCAAT | NC_045512.2 | 3′→5′ | 28771 | 28790 |
| 5303 | GCGTAGAAGCCTTTTGGCAA | NC_045512.2 | 3′→5′ | 28772 | 28791 |
| 5304 | TGCGTAGAAGCCTTTTGGCA | NC_045512.2 | 3′→5′ | 28773 | 28792 |
| 5305 | CTGCGTAGAAGCCTTTTGGC | NC_045512.2 | 3′→5′ | 28774 | 28793 |
| 5306 | TCTGCGTAGAAGCCTTTTGG | NC_045512.2 | 3′→5′ | 28775 | 28794 |
| 5307 | GCTTGACTGCCGCCTCTGCT | NC_045512.2 | 3′→5′ | 28799 | 28818 |
| 5308 | GGCTTGACTGCCGCCTCTGC | NC_045512.2 | 3′→5′ | 28800 | 28819 |
| 5309 | AGGCTTGACTGCCGCCTCTG | NC_045512.2 | 3′→5′ | 28801 | 28820 |
| 5310 | GAGGCTTGACTGCCGCCTCT | NC_045512.2 | 3′→5′ | 28802 | 28821 |
| 5311 | AGAGGCTTGACTGCCGCCTC | NC_045512.2 | 3′→5′ | 28803 | 28822 |
| 5312 | AAGAGGCTTGACTGCCGCCT | NC_045512.2 | 3′→5′ | 28804 | 28823 |
| 5313 | GAAGAGGCTTGACTGCCGCC | NC_045512.2 | 3′→5′ | 28805 | 28824 |
| 5314 | AGAAGAGGCTTGACTGCCGC | NC_045512.2 | 3′→5′ | 28806 | 28825 |
| 5315 | GAGAAGAGGCTTGACTGCCG | NC_045512.2 | 3′→5′ | 28807 | 28826 |
| 5316 | CGAGAAGAGGCTTGACTGCC | NC_045512.2 | 3′→5′ | 28808 | 28827 |
| 5317 | TCAAGCTGGTTCAATCTGTC | NC_045512.2 | 3′→5′ | 28946 | 28965 |
| 5318 | CTCAAGCTGGTTCAATCTGT | NC_045512.2 | 3′→5′ | 28947 | 28966 |
| 5319 | TCTCAAGCTGGTTCAATCTG | NC_045512.2 | 3′→5′ | 28948 | 28967 |
| 5320 | CTCTCAAGCTGGTTCAATCT | NC_045512.2 | 3′→5′ | 28949 | 28968 |
| 5321 | GCTCTCAAGCTGGTTCAATC | NC_045512.2 | 3′→5′ | 28950 | 28969 |
| 5322 | TGCTCTCAAGCTGGTTCAAT | NC_045512.2 | 3′→5′ | 28951 | 28970 |
| 5323 | TTGCTCTCAAGCTGGTTCAA | NC_045512.2 | 3′→5′ | 28952 | 28971 |
| 5324 | TTTGCTCTCAAGCTGGTTCA | NC_045512.2 | 3′→5′ | 28953 | 28972 |
| 5325 | TGTTGTTGGCCTTTACCAGA | NC_045512.2 | 3′→5′ | 28976 | 28995 |
| 5326 | TTGTTGTTGGCCTTTACCAG | NC_045512.2 | 3′→5′ | 28977 | 28996 |
| 5327 | GTTGTTGTTGGCCTTTACCA | NC_045512.2 | 3′→5′ | 28978 | 28997 |
| 5328 | TGTTGTTGTTGGCCTTTACC | NC_045512.2 | 3′→5′ | 28979 | 28998 |
| 5329 | TTGTTGTTGTTGGCCTTTAC | NC_045512.2 | 3′→5′ | 28980 | 28999 |
| 5330 | CTTGTTGTTGTTGGCCTTTA | NC_045512.2 | 3′→5′ | 28981 | 29000 |
| 5331 | CCTTGTTGTTGTTGGCCTTT | NC_045512.2 | 3′→5′ | 28982 | 29001 |
| 5332 | GCCTTGTTGTTGTTGGCCTT | NC_045512.2 | 3′→5′ | 28983 | 29002 |
| 5333 | GGCCTTGTTGTTGTTGGCCT | NC_045512.2 | 3′→5′ | 28984 | 29003 |
| 5334 | TGGCCTTGTTGTTGTTGGCC | NC_045512.2 | 3′→5′ | 28985 | 29004 |
| 5335 | TTGGCCTTGTTGTTGTTGGC | NC_045512.2 | 3′→5′ | 28986 | 29005 |
| 5336 | TTTGGCCTTGTTGTTGTTGG | NC_045512.2 | 3′→5′ | 28987 | 29006 |
| 5337 | GTTTGGCCTTGTTGTTGTTG | NC_045512.2 | 3′→5′ | 28988 | 29007 |
| 5338 | AGTTTGGCCTTGTTGTTGTT | NC_045512.2 | 3′→5′ | 28989 | 29008 |
| 5339 | CAGTTTGGCCTTGTTGTTGT | NC_045512.2 | 3′→5′ | 28990 | 29009 |
| 5340 | ACAGTTTGGCCTTGTTGTTG | NC_045512.2 | 3′→5′ | 28991 | 29010 |
| 5341 | GACAGTTTGGCCTTGTTGTT | NC_045512.2 | 3′→5′ | 28992 | 29011 |
| 5342 | TGACAGTTTGGCCTTGTTGT | NC_045512.2 | 3′→5′ | 28993 | 29012 |
| 5343 | GTGACAGTTTGGCCTTGTTG | NC_045512.2 | 3′→5′ | 28994 | 29013 |
| 5344 | AGTGACAGTTTGGCCTTGTT | NC_045512.2 | 3′→5′ | 28995 | 29014 |
| 5345 | TAGTGACAGTTTGGCCTTGT | NC_045512.2 | 3′→5′ | 28996 | 29015 |
| 5346 | TTAGTGACAGTTTGGCCTTG | NC_045512.2 | 3′→5′ | 28997 | 29016 |
| 5347 | CTTAGTGACAGTTTGGCCTT | NC_045512.2 | 3′→5′ | 28998 | 29017 |
| 5348 | TCTTAGTGACAGTTTGGCCT | NC_045512.2 | 3′→5′ | 28999 | 29018 |
| 5349 | TTCTTAGTGACAGTTTGGCC | NC_045512.2 | 3′→5′ | 29000 | 29019 |
| 5350 | TTTCTTAGTGACAGTTTGGC | NC_045512.2 | 3′→5′ | 29001 | 29020 |
| 5351 | ATTTCTTAGTGACAGTTTGG | NC_045512.2 | 3′→5′ | 29002 | 29021 |
| 5352 | GATTTCTTAGTGACAGTTTG | NC_045512.2 | 3′→5′ | 29003 | 29022 |
| 5353 | AGATTTCTTAGTGACAGTTT | NC_045512.2 | 3′→5′ | 29004 | 29023 |
| 5354 | CAGATTTCTTAGTGACAGTT | NC_045512.2 | 3′→5′ | 29005 | 29024 |
| 5355 | GCAGATTTCTTAGTGACAGT | NC_045512.2 | 3′→5′ | 29006 | 29025 |
| 5356 | AGCAGATTTCTTAGTGACAG | NC_045512.2 | 3′→5′ | 29007 | 29026 |
| 5357 | CAGCAGATTTCTTAGTGACA | NC_045512.2 | 3′→5′ | 29008 | 29027 |
| 5358 | GCAGCAGATTTCTTAGTGAC | NC_045512.2 | 3′→5′ | 29009 | 29028 |
| 5359 | AGCAGCAGATTTCTTAGTGA | NC_045512.2 | 3′→5′ | 29010 | 29029 |
| 5360 | CAGCAGCAGATTTCTTAGTG | NC_045512.2 | 3′→5′ | 29011 | 29030 |
| 5361 | TCAGCAGCAGATTTCTTAGT | NC_045512.2 | 3′→5′ | 29012 | 29031 |
| 5362 | CTCAGCAGCAGATTTCTTAG | NC_045512.2 | 3′→5′ | 29013 | 29032 |
| 5363 | CCTCAGCAGCAGATTTCTTA | NC_045512.2 | 3′→5′ | 29014 | 29033 |
| 5364 | GCCTCAGCAGCAGATTTCTT | NC_045512.2 | 3′→5′ | 29015 | 29034 |
| 5365 | TCAGTTCCTTGTCTGATTAG | NC_045512.2 | 3′→5′ | 29144 | 29163 |
| 5366 | ATCAGTTCCTTGTCTGATTA | NC_045512.2 | 3′→5′ | 29145 | 29164 |
| 5367 | AATCAGTTCCTTGTCTGATT | NC_045512.2 | 3′→5′ | 29146 | 29165 |
| 5368 | TAATCAGTTCCTTGTCTGAT | NC_045512.2 | 3′→5′ | 29147 | 29166 |
| 5369 | GTAATCAGTTCCTTGTCTGA | NC_045512.2 | 3′→5′ | 29148 | 29167 |
| 5370 | TGTAATCAGTTCCTTGTCTG | NC_045512.2 | 3′→5′ | 29149 | 29168 |
| 5371 | TTGTAATCAGTTCCTTGTCT | NC_045512.2 | 3′→5′ | 29150 | 29169 |
| 5372 | TTTGTAATCAGTTCCTTGTC | NC_045512.2 | 3′→5′ | 29151 | 29170 |
| 5373 | GTTTGTAATCAGTTCCTTGT | NC_045512.2 | 3′→5′ | 29152 | 29171 |
| 5374 | TGTTTGTAATCAGTTCCTTG | NC_045512.2 | 3′→5′ | 29153 | 29172 |
| 5375 | AATTGTGCAATTTGCGGCCA | NC_045512.2 | 3′→5′ | 29174 | 29193 |
| 5376 | AAATTGTGCAATTTGCGGCC | NC_045512.2 | 3′→5′ | 29175 | 29194 |
| 5377 | CAAATTGTGCAATTTGCGGC | NC_045512.2 | 3′→5′ | 29176 | 29195 |
| 5378 | GCAAATTGTGCAATTTGCGG | NC_045512.2 | 3′→5′ | 29177 | 29196 |
| 5379 | GTGACTTCCATGCCAATGCG | NC_045512.2 | 3′→5′ | 29228 | 29247 |
| 5380 | TGTGACTTCCATGCCAATGC | NC_045512.2 | 3′→5′ | 29229 | 29248 |
| 5381 | GTGTGACTTCCATGCCAATG | NC_045512.2 | 3′→5′ | 29230 | 29249 |
| 5382 | GGTGTGACTTCCATGCCAAT | NC_045512.2 | 3′→5′ | 29231 | 29250 |
| 5383 | AGGTGTGACTTCCATGCCAA | NC_045512.2 | 3′→5′ | 29232 | 29251 |
| 5384 | AAGGTGTGACTTCCATGCCA | NC_045512.2 | 3′→5′ | 29233 | 29252 |
| 5385 | GAAGGTGTGACTTCCATGCC | NC_045512.2 | 3′→5′ | 29234 | 29253 |
| 5386 | CGAAGGTGTGACTTCCATGC | NC_045512.2 | 3′→5′ | 29235 | 29254 |
| 5387 | CCGAAGGTGTGACTTCCATG | NC_045512.2 | 3′→5′ | 29236 | 29255 |
| 5388 | CCCGAAGGTGTGACTTCCAT | NC_045512.2 | 3′→5′ | 29237 | 29256 |
| 5389 | TCCCGAAGGTGTGACTTCCA | NC_045512.2 | 3′→5′ | 29238 | 29257 |
| 5390 | TTCCCGAAGGTGTGACTTCC | NC_045512.2 | 3′→5′ | 29239 | 29258 |
| 5391 | GTTCCCGAAGGTGTGACTTC | NC_045512.2 | 3′→5′ | 29240 | 29259 |
| 5392 | GGATCTTTGTCATCCAATTT | NC_045512.2 | 3′→5′ | 29285 | 29304 |
| 5393 | TGGATCTTTGTCATCCAATT | NC_045512.2 | 3′→5′ | 29286 | 29305 |
| 5394 | AATGTTTTGTATGCGTCAAT | NC_045512.2 | 3′→5′ | 29342 | 29361 |
| 5395 | GAATGTTTTGTATGCGTCAA | NC_045512.2 | 3′→5′ | 29343 | 29362 |
| 5396 | GGAATGTTTTGTATGCGTCA | NC_045512.2 | 3′→5′ | 29344 | 29363 |
| 5397 | GGGAATGTTTTGTATGCGTC | NC_045512.2 | 3′→5′ | 29345 | 29364 |
| 5398 | TGGGAATGTTTTGTATGCGT | NC_045512.2 | 3′→5′ | 29346 | 29365 |
| 5399 | GTGGGAATGTTTTGTATGCG | NC_045512.2 | 3′→5′ | 29347 | 29366 |
| 5400 | GGTGGGAATGTTTTGTATGC | NC_045512.2 | 3′→5′ | 29348 | 29367 |
| 5401 | TGGTGGGAATGTTTTGTATG | NC_045512.2 | 3′→5′ | 29349 | 29368 |
| 5402 | TTGGTGGGAATGTTTTGTAT | NC_045512.2 | 3′→5′ | 29350 | 29369 |
| 5403 | GTTGGTGGGAATGTTTTGTA | NC_045512.2 | 3′→5′ | 29351 | 29370 |
| 5404 | TGTTGGTGGGAATGTTTTGT | NC_045512.2 | 3′→5′ | 29352 | 29371 |
| 5405 | CTGTTGGTGGGAATGTTTTG | NC_045512.2 | 3′→5′ | 29353 | 29372 |
| 5406 | TCTGTTGGTGGGAATGTTTT | NC_045512.2 | 3′→5′ | 29354 | 29373 |
| 5407 | CTCTGTTGGTGGGAATGTTT | NC_045512.2 | 3′→5′ | 29355 | 29374 |
| 5408 | GCTCTGTTGGTGGGAATGTT | NC_045512.2 | 3′→5′ | 29356 | 29375 |
| 5409 | GGCTCTGTTGGTGGGAATGT | NC_045512.2 | 3′→5′ | 29357 | 29376 |
| 5410 | AGGCTCTGTTGGTGGGAATG | NC_045512.2 | 3′→5′ | 29358 | 29377 |
| 5411 | TAGGCTCTGTTGGTGGGAAT | NC_045512.2 | 3′→5′ | 29359 | 29378 |
| 5412 | TTAGGCTCTGTTGGTGGGAA | NC_045512.2 | 3′→5′ | 29360 | 29379 |
| 5413 | TTTAGGCTCTGTTGGTGGGA | NC_045512.2 | 3′→5′ | 29361 | 29380 |
| 5414 | TTTTAGGCTCTGTTGGTGGG | NC_045512.2 | 3′→5′ | 29362 | 29381 |
| 5415 | TTTTTAGGCTCTGTTGGTGG | NC_045512.2 | 3′→5′ | 29363 | 29382 |
| 5416 | CTTTTTAGGCTCTGTTGGTG | NC_045512.2 | 3′→5′ | 29364 | 29383 |
| 5417 | CCTTTTTAGGCTCTGTTGGT | NC_045512.2 | 3′→5′ | 29365 | 29384 |
| 5418 | TCCTTTTTAGGCTCTGTTGG | NC_045512.2 | 3′→5′ | 29366 | 29385 |
| 5419 | GTCCTTTTTAGGCTCTGTTG | NC_045512.2 | 3′→5′ | 29367 | 29386 |
| 5420 | TGTCCTTTTTAGGCTCTGTT | NC_045512.2 | 3′→5′ | 29368 | 29387 |
| 5421 | TTGTCCTTTTTAGGCTCTGT | NC_045512.2 | 3′→5′ | 29369 | 29388 |
| 5422 | TTTGTCCTTTTTAGGCTCTG | NC_045512.2 | 3′→5′ | 29370 | 29389 |
| 5423 | TTTTGTCCTTTTTAGGCTCT | NC_045512.2 | 3′→5′ | 29371 | 29390 |
| 5424 | TTTTTGTCCTTTTTAGGCTC | NC_045512.2 | 3′→5′ | 29372 | 29391 |
| 5425 | CTTTTTGTCCTTTTTAGGCT | NC_045512.2 | 3′→5′ | 29373 | 29392 |
| 5426 | TCTTTTTGTCCTTTTTAGGC | NC_045512.2 | 3′→5′ | 29374 | 29393 |
| 5427 | TTCTTTTTGTCCTTTTTAGG | NC_045512.2 | 3′→5′ | 29375 | 29394 |
| 5428 | GCAGGAAGAAGAGTCACAGT | NC_045512.2 | 3′→5′ | 29444 | 29463 |
| 5429 | CCCATCTGCCTTGTGTGGTC | NC_045512.2 | 3′→5′ | 29543 | 29562 |
| 5430 | GCCCATCTGCCTTGTGTGGT | NC_045512.2 | 3′→5′ | 29544 | 29563 |
| 5431 | AGCCCATCTGCCTTGTGTGG | NC_045512.2 | 3′→5′ | 29545 | 29564 |
| 5432 | TAGCCCATCTGCCTTGTGTG | NC_045512.2 | 3′→5′ | 29546 | 29565 |
| 5433 | ATAGCCCATCTGCCTTGTGT | NC_045512.2 | 3′→5′ | 29547 | 29566 |
| 5434 | TCTGCACAAGAGTAGACTAT | NC_045512.2 | 3′→5′ | 29598 | 29617 |
| 5435 | TTCTGCACAAGAGTAGACTA | NC_045512.2 | 3′→5′ | 29599 | 29618 |
| 5436 | ATTCTGCACAAGAGTAGACT | NC_045512.2 | 3′→5′ | 29600 | 29619 |
| 5437 | CATTCTGCACAAGAGTAGAC | NC_045512.2 | 3′→5′ | 29601 | 29620 |
| 5438 | TCATTCTGCACAAGAGTAGA | NC_045512.2 | 3′→5′ | 29602 | 29621 |
| 5439 | TTCATTCTGCACAAGAGTAG | NC_045512.2 | 3′→5′ | 29603 | 29622 |
| 5440 | ATTCATTCTGCACAAGAGTA | NC_045512.2 | 3′→5′ | 29604 | 29623 |
| 5441 | AATTCATTCTGCACAAGAGT | NC_045512.2 | 3′→5′ | 29605 | 29624 |
| 5442 | GAATTCATTCTGCACAAGAG | NC_045512.2 | 3′→5′ | 29606 | 29625 |
| 5443 | AGAATTCATTCTGCACAAGA | NC_045512.2 | 3′→5′ | 29607 | 29626 |
| 5444 | GAGAATTCATTCTGCACAAG | NC_045512.2 | 3′→5′ | 29608 | 29627 |
| 5445 | CGAGAATTCATTCTGCACAA | NC_045512.2 | 3′→5′ | 29609 | 29628 |
| 5446 | ACGAGAATTCATTCTGCACA | NC_045512.2 | 3′→5′ | 29610 | 29629 |
| 5447 | TACGAGAATTCATTCTGCAC | NC_045512.2 | 3′→5′ | 29611 | 29630 |
| 5448 | TGTGAGATTAAAGTTAACTA | NC_045512.2 | 3′→5′ | 29652 | 29671 |
| 5449 | ATGTGAGATTAAAGTTAACT | NC_045512.2 | 3′→5′ | 29653 | 29672 |
| 5450 | TATGTGAGATTAAAGTTAAC | NC_045512.2 | 3′→5′ | 29654 | 29673 |
| 5451 | CTATGTGAGATTAAAGTTAA | NC_045512.2 | 3′→5′ | 29655 | 29674 |
| 5452 | GCTATGTGAGATTAAAGTTA | NC_045512.2 | 3′→5′ | 29656 | 29675 |
| 5453 | TGCTATGTGAGATTAAAGTT | NC_045512.2 | 3′→5′ | 29657 | 29676 |
| 5454 | TTGCTATGTGAGATTAAAGT | NC_045512.2 | 3′→5′ | 29658 | 29677 |
| 5455 | ATTGCTATGTGAGATTAAAG | NC_045512.2 | 3′→5′ | 29659 | 29678 |
| 5456 | GATTGCTATGTGAGATTAAA | NC_045512.2 | 3′→5′ | 29660 | 29679 |
| 5457 | AGATTGCTATGTGAGATTAA | NC_045512.2 | 3′→5′ | 29661 | 29680 |
| 5458 | AAGATTGCTATGTGAGATTA | NC_045512.2 | 3′→5′ | 29662 | 29681 |
| 5459 | AAAGATTGCTATGTGAGATT | NC_045512.2 | 3′→5′ | 29663 | 29682 |
| 5460 | TAAAGATTGCTATGTGAGAT | NC_045512.2 | 3′→5′ | 29664 | 29683 |
| 5461 | TTAAAGATTGCTATGTGAGA | NC_045512.2 | 3′→5′ | 29665 | 29684 |
| 5462 | ATTAAAGATTGCTATGTGAG | NC_045512.2 | 3′→5′ | 29666 | 29685 |
| 5463 | GATTAAAGATTGCTATGTGA | NC_045512.2 | 3′→5′ | 29667 | 29686 |
| 5464 | TGATTAAAGATTGCTATGTG | NC_045512.2 | 3′→5′ | 29668 | 29687 |
| 5465 | GTCCTCCCTAATGTTACACA | NC_045512.2 | 3′→5′ | 29689 | 29708 |
| 5466 | AGTCCTCCCTAATGTTACAC | NC_045512.2 | 3′→5′ | 29690 | 29709 |
| 5467 | AAGTCCTCCCTAATGTTACA | NC_045512.2 | 3′→5′ | 29691 | 29710 |
| 5468 | CAAGTCCTCCCTAATGTTAC | NC_045512.2 | 3′→5′ | 29692 | 29711 |
| 5469 | TCAAGTCCTCCCTAATGTTA | NC_045512.2 | 3′→5′ | 29693 | 29712 |
| 5470 | TTCAAGTCCTCCCTAATGTT | NC_045512.2 | 3′→5′ | 29694 | 29713 |
| 5471 | TTTCAAGTCCTCCCTAATGT | NC_045512.2 | 3′→5′ | 29695 | 29714 |
| 5472 | CTTTCAAGTCCTCCCTAATG | NC_045512.2 | 3′→5′ | 29696 | 29715 |
| 5473 | TCTTTCAAGTCCTCCCTAAT | NC_045512.2 | 3′→5′ | 29697 | 29716 |
| 5474 | CTCTTTCAAGTCCTCCCTAA | NC_045512.2 | 3′→5′ | 29698 | 29717 |
| 5475 | GCTCTTTCAAGTCCTCCCTA | NC_045512.2 | 3′→5′ | 29699 | 29718 |
| 5476 | GGCTCTTTCAAGTCCTCCCT | NC_045512.2 | 3′→5′ | 29700 | 29719 |
| 5477 | TGGCTCTTTCAAGTCCTCCC | NC_045512.2 | 3′→5′ | 29701 | 29720 |
| 5478 | GTGGCTCTTTCAAGTCCTCC | NC_045512.2 | 3′→5′ | 29702 | 29721 |
| 5479 | GGTGGCTCTTTCAAGTCCTC | NC_045512.2 | 3′→5′ | 29703 | 29722 |
| 5480 | TGGTGGCTCTTTCAAGTCCT | NC_045512.2 | 3′→5′ | 29704 | 29723 |
| 5481 | GTGGTGGCTCTTTCAAGTCC | NC_045512.2 | 3′→5′ | 29705 | 29724 |
| 5482 | TGTGGTGGCTCTTTCAAGTC | NC_045512.2 | 3′→5′ | 29706 | 29725 |
| 5483 | ATGTGGTGGCTCTTTCAAGT | NC_045512.2 | 3′→5′ | 29707 | 29726 |
| 5484 | AATGTGGTGGCTCTTTCAAG | NC_045512.2 | 3′→5′ | 29708 | 29727 |
| 5485 | AAATGTGGTGGCTCTTTCAA | NC_045512.2 | 3′→5′ | 29709 | 29728 |
| 5486 | AAAATGTGGTGGCTCTTTCA | NC_045512.2 | 3′→5′ | 29710 | 29729 |
| 5487 | GAAAATGTGGTGGCTCTTTC | NC_045512.2 | 3′→5′ | 29711 | 29730 |
| 5488 | TGAAAATGTGGTGGCTCTTT | NC_045512.2 | 3′→5′ | 29712 | 29731 |
| 5489 | TCGTACTCCGCGTGGCCTCG | NC_045512.2 | 3′→5′ | 29733 | 29752 |
| 5490 | ATCGTACTCCGCGTGGCCTC | NC_045512.2 | 3′→5′ | 29734 | 29753 |
| 5491 | GATCGTACTCCGCGTGGCCT | NC_045512.2 | 3′→5′ | 29735 | 29754 |
| 5492 | CGATCGTACTCCGCGTGGCC | NC_045512.2 | 3′→5′ | 29736 | 29755 |
| 5493 | TCGATCGTACTCCGCGTGGC | NC_045512.2 | 3′→5′ | 29737 | 29756 |
| 5494 | CTCGATCGTACTCCGCGTGG | NC_045512.2 | 3′→5′ | 29738 | 29757 |
| 5495 | AGGCAGCTCTCCCTAGCATT | NC_045512.2 | 3′→5′ | 29770 | 29789 |
| 5496 | TAGGCAGCTCTCCCTAGCAT | NC_045512.2 | 3′→5′ | 29771 | 29790 |
| 5497 | ATAGGCAGCTCTCCCTAGCA | NC_045512.2 | 3′→5′ | 29772 | 29791 |
| 5498 | TATAGGCAGCTCTCCCTAGC | NC_045512.2 | 3′→5′ | 29773 | 29792 |
| 5499 | ATATAGGCAGCTCTCCCTAG | NC_045512.2 | 3′→5′ | 29774 | 29793 |
| 5500 | CATATAGGCAGCTCTCCCTA | NC_045512.2 | 3′→5′ | 29775 | 29794 |
| 5501 | CCATATAGGCAGCTCTCCCT | NC_045512.2 | 3′→5′ | 29776 | 29795 |
| 5502 | TCCATATAGGCAGCTCTCCC | NC_045512.2 | 3′→5′ | 29777 | 29796 |
| 5503 | TTCCATATAGGCAGCTCTCC | NC_045512.2 | 3′→5′ | 29778 | 29797 |
| 5504 | CTTCCATATAGGCAGCTCTC | NC_045512.2 | 3′→5′ | 29779 | 29798 |
| 5505 | TCTTCCATATAGGCAGCTCT | NC_045512.2 | 3′→5′ | 29780 | 29799 |
| 5506 | CTCTTCCATATAGGCAGCTC | NC_045512.2 | 3′→5′ | 29781 | 29800 |
| 5507 | GCTCTTCCATATAGGCAGCT | NC_045512.2 | 3′→5′ | 29782 | 29801 |
| 5508 | GGCTCTTCCATATAGGCAGC | NC_045512.2 | 3′→5′ | 29783 | 29802 |
| 5509 | GGGCTCTTCCATATAGGCAG | NC_045512.2 | 3′→5′ | 29784 | 29803 |
| 5510 | AGGGCTCTTCCATATAGGCA | NC_045512.2 | 3′→5′ | 29785 | 29804 |
| 5511 | TAGGGCTCTTCCATATAGGC | NC_045512.2 | 3′→5′ | 29786 | 29805 |
| 5512 | TTAGGGCTCTTCCATATAGG | NC_045512.2 | 3′→5′ | 29787 | 29806 |
| 5513 | ATTAGGGCTCTTCCATATAG | NC_045512.2 | 3′→5′ | 29788 | 29807 |
| 5514 | CATTAGGGCTCTTCCATATA | NC_045512.2 | 3′→5′ | 29789 | 29808 |
| 5515 | ACATTAGGGCTCTTCCATAT | NC_045512.2 | 3′→5′ | 29790 | 29809 |
| 5516 | CACATTAGGGCTCTTCCATA | NC_045512.2 | 3′→5′ | 29791 | 29810 |
| 5517 | ACACATTAGGGCTCTTCCAT | NC_045512.2 | 3′→5′ | 29792 | 29811 |
| 5518 | TACACATTAGGGCTCTTCCA | NC_045512.2 | 3′→5′ | 29793 | 29812 |
| 5519 | TTACACATTAGGGCTCTTCC | NC_045512.2 | 3′→5′ | 29794 | 29813 |
| 5520 | TTTACACATTAGGGCTCTTC | NC_045512.2 | 3′→5′ | 29795 | 29814 |
| 5521 | TTTTACACATTAGGGCTCTT | NC_045512.2 | 3′→5′ | 29796 | 29815 |
| 5522 | ATTTTACACATTAGGGCTCT | NC_045512.2 | 3′→5′ | 29797 | 29816 |
| 5523 | AATTTTACACATTAGGGCTC | NC_045512.2 | 3′→5′ | 29798 | 29817 |
| 5524 | TAATTTTACACATTAGGGCT | NC_045512.2 | 3′→5′ | 29799 | 29818 |
| 5525 | TTAATTTTACACATTAGGGC | NC_045512.2 | 3′→5′ | 29800 | 29819 |
| 5526 | ATTAATTTTACACATTAGGG | NC_045512.2 | 3′→5′ | 29801 | 29820 |
| 5527 | AATTAATTTTACACATTAGG | NC_045512.2 | 3′→5′ | 29802 | 29821 |
| 5528 | AAATTAATTTTACACATTAG | NC_045512.2 | 3′→5′ | 29803 | 29822 |
| 5529 | AAAATTAATTTTACACATTA | NC_045512.2 | 3′→5′ | 29804 | 29823 |
| 5530 | TAAAATTAATTTTACACATT | NC_045512.2 | 3′→5′ | 29805 | 29824 |
| 5531 | CTAAAATTAATTTTACACAT | NC_045512.2 | 3′→5′ | 29806 | 29825 |
| 5532 | ACTAAAATTAATTTTACACA | NC_045512.2 | 3′→5′ | 29807 | 29826 |
| 5533 | TACTAAAATTAATTTTACAC | NC_045512.2 | 3′→5′ | 29808 | 29827 |
| 5534 | CTACTAAAATTAATTTTACA | NC_045512.2 | 3′→5′ | 29809 | 29828 |

For the purposes of the present disclosure, it is to be understood that Table 1 discloses not only SEQ ID NOs: 1-5534, but also the complements thereof. In some embodiments, the complements of any one of SEQ ID NOs: 1-5534 may be utilized for preparing a coronavirus-specific ASO. In some embodiments, the ASOs of the disclosure have a sequence that differs from an ASO of Table 1 by one nucleoside. In other embodiments, the ASO has a sequence that differs from an ASO of Table 1 by 1, 2, 3 or 4 nucleosides. In some embodiments, the nucleotide sequence is at least 90% identical to a nucleotide sequence selected from Table 1 In some embodiments, the ASOs of the disclosure have a sequence of Table 1, but one T in the central region is replaced by (2s)T, one C in the central region is replaced by (5OH)C, and/or one A is replaced by (8NH)A in the central region. In some embodiments, the ASOs of the disclosure have a sequence of Table 1, but with one or two ScpBNA, AmNA, or GuNA in the 5′ wing portion. In some embodiments, the ASOs of the disclosure have a sequence of Table 1, but with one or two ScpBNA, AmNA, or GuNA in the 3′ wing portion. In some embodiments, the ASOs of the disclosure have a sequence of Table 1, but with a mA or mU appended to the 5′ end of the sequence.

The list of ASO sequences provided in Table 1 is not intended to be limiting, and a skilled artisan will understand that other similar ASO may be derived from other coronaviruses or from other portions of GenBank Accession NC_045512.2. For example, in some embodiments, an ASO may possess about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or about 100% homology or identity to any of the sequences disclosed in Table 1.

In some embodiments, the compounds of the disclosure have a sequence that differs from an ASO sequences disclosed in Table 1 by one nucleoside. In other embodiments, the ASO has a sequence that differs from an ASO of Table 1 by 1, 2, 3, or 4 nucleosides. In some embodiments, the nucleotide sequence is at least 90% identical to a nucleotide sequence selected from Table 1. In some embodiments, the compounds of the disclosure have a sequence of Table 1, but one T in the central region is replaced by (2s)T, one C in the central region is replaced by (5OH)C, and/or one A is replaced by (8NH)A in the central region. In some embodiments, the compounds of the disclosure have a sequence of Table 1, but with one or two ScpBNA, AmNA, or GuNA in the 5′ wing portion. In some embodiments, the compounds of the disclosure have a sequence of Table 1, but with one or two ScpBNA, AmNA, or GuNA in the 3′ wing portion. In some embodiments, the compounds of the disclosure have a sequence of Table 1, but with one or two or more LNA in the 5′ wing portion. In some embodiments, the compounds of the disclosure have a sequence of Table 1, but with one or two or more LNA in the 3′ wing portion. In some embodiments, the compounds of the disclosure have a sequence of Table 1, but with a mA or mU appended to the 5′ end of the sequence. In some embodiments, the compounds of the disclosure have a sequence of Table 1, but with a mA or mU appended to the 5′ end of the sequence that links to a conjugation moiety (i.e., a targeting moiety). In some embodiments, the compounds of the disclosure have a sequence of Table 1, but with a mA or mU appended to the 3′ end of the sequence. In some embodiments, the compounds of the disclosure have a sequence of Table 1, but with a mA or mU appended to the 3′ end of the sequence that links to a conjugation moiety (i.e., a targeting moiety).

In some embodiments, the sequence of an ASO may comprise 8, 9, 10, 11, 12, 13, 14, 15, or 16 or more consecutive nucleotides of any one of the coronavirus genomes shown in Table 2 at the end of the Detailed Description Section or a sequence possessing about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or about 100% homology or identity with a sequence comprising, 9, 10, 11, 12, 13, 14, 15, or 16 or more consecutive nucleotides of any one of the coronavirus genomes shown in Table 2. In the foregoing embodiments, the ASO sequence may comprise one or more modified, locked, conjugated, or linked nucleotides/nucleosides in addition to or in place of any of the 8, 9, 10, 11, 12, 13, 14, 15, or 16 or more consecutive nucleotides of any one of the coronavirus genomes shown in Table 2 or homologs thereof.

In some embodiments, the disclosed ASO molecules comprise one or more modified nucleotides. In some embodiments, any of the ASO molecules disclosed herein comprise 1, 2, 3, 4, 5, 6, 7, 8, 9, 10, 11, 12, 13, 14, 15, 16, 17, 18, 19, 20, 21, or 22. In some embodiments, 1%, 2%, 3%, 4%, 5%, 10%, 15%, 20%, 25%, 30%, 35%, 40%, 45%, 50%, 55%, 60%, 70%, 75%, 80%, 85%, 86%, 87%, 88%, 90%, 91%, 92%, 93%, 94%, 95%, 96%, 97%, 98%, 99% or 100% of the nucleotides in the ASO molecule are modified nucleotides.

In some embodiments, the disclosed ASO molecules comprise one or more locked nucleotides. In some embodiments, any of the ASO molecules disclosed herein comprise 1, 2, 3, 4, 5, 6, 7, 8, 9, 10, 11, 12, 13, 14, 15, 16, 17, 18, 19, 20, 21, or 22. In some embodiments, 1%, 2%, 3%, 4%, 5%, 10%, 15%, 20%, 25%, 30%, 35%, 40%, 45%, 50%, 55%, 60%, 70%, 75%, 80%, 85%, 86%, 87%, 88%, 90%, 91%, 92%, 93%, 94%, 95%, 96%, 97%, 98%, 99% or 100% of the nucleotides in the ASO molecule are locked nucleotides.

In some embodiments, the ASO is modified at one or more end by a vinyl phosphonate moiety, such as a 5′-vinyl phosphonate moiety.

### Conjugated or Targeting Moiety

The present disclosure is also directed to additional components conjugated to the ASO such as conjugated moieties (e.g., a targeting moieties) and oligonucleotides modified at one or more end.

In some embodiments, the disclosed ASO molecules comprise a conjugated moiety (i.e., a targeting moiety). The conjugated moiety may improve or alter the pharmacokinetics of the ASO and/or target the ASO to a particular cell or type of cell (e.g., lung cells). In some embodiments, the conjugated/targeting moiety may comprise a carbohydrate, such as a monosaccharide, disaccharide, trisaccharide, tetrasaccharide, oligosaccharide, and polysaccharide.

In some embodiments, the conjugated/targeting moiety is attached to the 3′ end of the ASO. In some embodiments, the conjugated/targeting moiety is attached to the 3′ end of the ASO via 1, 2, 3, 4, or 5 or more linkers. In some embodiments, the conjugated/targeting moiety is attached to the 5′ end of the ASO. In some embodiments, the conjugated/targeting moiety is attached to the 5′ end of the ASO via 1, 2, 3, 4, or 5 or more linkers. In some embodiments, the one or more linkers are independently selected from the group consisting of a phosphodiester linker, phosphorothioate linker, and phosphorodithioate linker.

In some embodiments, the ASO contains a conjugated/targeting moiety at the 5′-end, the 3′-end, or both ends of the ASO.

In some embodiments, the ASO is modified at one or more end by a vinyl phosphonate moiety, such as a 5′-vinyl phosphonate moiety.

### Specific Embodiments

The present disclosure provides numerous ASOs that can be used to treat or prevent viral infections, specifically coronavirus (e.g., SARS-CoV-2) infections, such as COVID-19. Table 3, below, provides a non-limiting list of ASOs that incorporate the various elements and sequences described in the preceding section. Those of skill in the art will understand that other exemplary ASOs can be constructed by combining the sequences disclosed in Table 1 (or fragments of the sequences disclosed in Table 2) with the elements, sequences, and designs described in the preceding section.

TABLE 3
Exemplary ASOs

| | SEQ ID | |
|---|---|---|
| Name | NO: | Sequence (5′ to 3′) |
| ASO- | 5539 | lnApslnTpsln(5m)CpslnAps(5m)Cps(2s)Tps(5m)CpsTpsTpsApsGpsGpsG |
| 000 | | pslnApslnApslnT |
| ASO- | 5540 | ln(5m)CpslnApslnTpsln(5m)CpsAps(5oh)CpsTps(5m)CpsTpsTpsApsGps |
| 001 | | GpslnGpslnApslnA |
| ASO- | 5541 | ln(5m)Cpsln(5m)CpslnApslnTps(5m)Cps(8nh)Aps(5m)CpsTps(5m)CpsT |
| 002 | | psTpsApsGpslnGpslnGpslnA |
| ASO- | 5542 | lnGpslnApsln(5m)CpslnGpsGps(8nh)Gps(5m)CpsTpsGps(5m)CpsAps |
| 003 | | (5m)CpsTpslnTpslnApsln(5m)C |
| ASO- | 5543 | lnTpslnGpsln(5m)CpslnAps(5m)Cps(2s)TpsApsApsTpsGpsGps(5m)CpsA |
| 004 | | pslnTpslnApsln(5m)C |
| ASO- | 5544 | lnTpslnGpsln(5m)CpslnGpsAps(8nh)Gps(5m)CpsTps(5m)CpsTpsApsTps |
| 005 | | Tpsln(5m)CpslnTpslnT |
| ASO- | 5545 | lnGpslnTpslnGpsln(5m)CpsGps(8nh)ApsGps(5m)CpsTps(5m)CpsTpsAps |
| 006 | | TpslnTpsln(5m)CpslnT |
| ASO- | 5546 | ln(5m)CpslnApslnTpslnApsGps(2s)TpsAps(5m)CpsTpsAps(5m)CpsApsG |
| 007 | | pslnApslnTpslnA |
| ASO- | 5547 | lnTpslnTpslnApslnGps(5m)Cps(8nh)ApsTpsApsApsGps(5m)CpsApsGpsl |
| 008 | | nTpslnTpslnG |
| ASO- | 5548 | lnApslnApsln(5m)CpslnApsTps(8nh)GpsTps(5m)CpsTpsGpsGpsAps(5m) |
| 009 | | Cpsln(5m)CpslnTpslnA |
| ASO- | 5549 | lnTpslnApslnTpsln(5m)CpsAps(8nh)GpsAps(5m)CpsApsTpsTpsApsTpsl |
| 010 | | nGpsln(5m)CpslnA |
| ASO- | 5550 | ln(5m)CpslnTpslnApslnTps(5m)Cps(8nh)ApsGpsAps(5m)CpsApsTpsTps |
| 011 | | ApslnTpslnGpsln(5m)C |
| ASO- | 5551 | lnTpsln(5m)CpslnTpslnApsTps(5oh)CpsApsGpsAps(5m)CpsApsTpsTpsl |
| 012 | | nApslnTpslnG |
| ASO- | 5552 | ln(5m)CpslnTpsln(5m)CpslnTpsAps(2s)Tps(5m)CpsApsGpsAps(5m)Cps |
| 013 | | ApsTpslnTpslnApslnT |
| ASO- | 5553 | ln(5m)CpslnTpslnApslnTpsTps(8nh)ApsAps(5m)CpsGpsTpsAps(5m)Cps |
| 014 | | (5m)CpslnTpslnGpslnT |
| ASO- | 5554 | lnGpslnTpslnApsln(5m)CpsGps(5oh)CpsAps(5m)CpsAps(5m)CpsApsAps |
| 015 | | Tpsln(5m)CpslnGpslnA |
| ASO- | 5555 | ln(5m)CpslnApslnGpslnTpsAps(5oh)CpsGps(5m)CpsAps(5m)CpsAps |
| 016 | | (5m)CpsApslnApslnTpsln(5m)C |
| ASO- | 5556 | lnGpsln(5m)Cpsln(5m)CpslnApsAps(2s)TpsTpsTpsGpsGpsTps(5m)CpsA |
| 017 | | pslnTpsln(5m)CpslnT |
| ASO- | 5557 | lnApslnApslnTpslnTpsGps(2s)TpsGps(5m)CpsApsApsTpsTpsTpslnGpsl |
| 018 | | n(5m)CpslnG |
| ASO- | 5558 | ln(5m)Cpsln(5m)CpslnApslnTpsAps(2s)TpsApsGpsGps(5m)CpsApsGps |
| 019 | | (5m)CpslnTpsln(5m)CpslnT |
| ASO- | 5559 | lnTpsln(5m)Cpsln(5m)CpslnApsTps(8nh)ApsTpsApsGpsGps(5m)CpsAps |
| 020 | | Gpsln(5m)CpslnTpsln(5m)C |
| ASO- | 5560 | lnTpslnTpslnApslnGpsGps(8nh)Gps(5m)CpsTps(5m)CpsTpsTps(5m)Cps |
| 021 | | (5m)CpslnApslnTpslnA |
| ASO- | 5561 | lnApslnTpsln(5m)CpsAps(5m)Cps(2s)Tps(5m)CpsTpsTpsApsGpsGpslnG |
| 022 | | pslnApslnApslnT |
| ASO- | 5562 | ln(5m)CpslnApslnTps(5m)CpsAps(5oh)CpsTps(5m)CpsTpsTpsApsGpsln |
| 023 | | GpslnGpslnApslnA |
| ASO- | 5563 | ln(5m)Cpsln(5m)CpslnApsTps(5m)Cps(8nh)Aps(5m)CpsTps(5m)CpsTps |
| 024 | | TpsApslnGpslnGpslnGpslnA |
| ASO- | 5564 | lnGpslnApsln(5m)CpsGpsGps(8nh)Gps(5m)CpsTpsGps(5m)CpsAps(5m)C |
| 025 | | pslnTpslnTpslnApsln(5m)C |
| ASO- | 5565 | lnTpslnGpsln(5m)CpsAps(5m)Cps(2s)TpsApsApsTpsGpsGps(5m)CpslnA |
| 026 | | pslnTpslnApsln(5m)C |
| ASO- | 5566 | lnTpslnGpsln(5m)CpsGpsAps(8nh)Gps(5m)CpsTps(5m)CpsTpsApsTpsln |
| 027 | | Tpsln(5m)CpslnTpslnT |
| ASO- | 5567 | lnGpslnTpslnGps(5m)CpsGps(8nh)ApsGps(5m)CpsTps(5m)CpsTpsApsln |
| 028 | | TpslnTpsln(5m)CpslnT |
| ASO- | 5568 | ln(5m)CpslnApslnTpsApsGps(2s)TpsAps(5m)CpsTpsAps(5m)CpsApslnG |
| 029 | | pslnApslnTpslnA |
| ASO- | 5569 | lnTpslnTpslnApsGps(5m)Cps(8nh)ApsTpsApsApsGps(5m)CpsApslnGps |
| 030 | | lnTpslnTpslnG |
| ASO- | 5570 | lnApslnApsln(5m)CpsApsTps(8nh)GpsTps(5m)CpsTpsGpsGpsApsln(5m) |
| 031 | | Cpsln(5m)CpslnTpslnA |
| ASO- | 5571 | lnTpslnApslnTps(5m)CpsAps(8nh)GpsAps(5m)CpsApsTpsTpsApslnTps |
| 032 | | lnGpsln(5m)CpslnA |
| ASO- | 5572 | ln(5m)CpslnTpslnApsTps(5m)Cps(8nh)ApsGpsAps(5m)CpsApsTpsTpsln |
| 033 | | ApslnTpslnGpsln(5m)C |
| ASO- | 5573 | lnTpsln(5m)CpslnTpsApsTps(5oh)CpsApsGpsAps(5m)CpsApsTpslnTps |
| 034 | | lnApslnTpslnG |
| ASO- | 5574 | ln(5m)CpslnTpsln(5m)CpsTpsAps(2s)Tps(5m)CpsApsGpsAps(5m)CpsA |
| 035 | | pslnTpslnTpslnApslnT |
| ASO- | 5575 | ln(5m)CpslnTpslnApsTpsTps(8nh)ApsAps(5m)CpsGpsTpsAps(5m)Cpsln |
| 036 | | (5m)CpslnTpslnGpslnT |
| ASO- | 5576 | lnGpslnTpslnAps(5m)CpsGps(5oh)CpsAps(5m)CpsAps(5m)CpsApsApsln |
| 037 | | Tpsln(5m)CpslnGpslnA |
| ASO- | 5577 | lnGpslnTpslnAps(5m)CpsGps(5oh)CpsAps(5m)CpsAps(5m)CpsApsApsln |
| 038 | | Tpsln(5m)CpslnGpslnA |
| ASO- | 5578 | ln(5m)CpslnApslnGpsTpsAps(5oh)CpsGps(5m)CpsAps(5m)CpsAps(5m)C |
| 039 | | pslnApslnApslnTpsln(5m)C |
| ASO- | 5579 | lnGpsln(5m)Cpsln(5m)CpsApsAps(2s)TpsTpsTpsGpsGpsTps(5m)CpslnA |
| 040 | | pslnTpsln(5m)CpslnT |
| ASO- | 5580 | lnApslnApslnTpsTpsGps(2s)TpsGps(5m)CpsApsApsTpsTpslnTpslnGps |
| 041 | | ln(5m)CpslnG |
| ASO- | 5581 | ln(5m)Cpsln(5m)CpslnApsTpsAps(2s)TpsApsGpsGps(5m)CpsApsGpsln |
| 042 | | (5m)CpslnTpsln(5m)CpslnT |
| ASO- | 5582 | lnTpsln(5m)Cpsln(5m)CpsApsTps(8nh)ApsTpsApsGpsGps(5m)CpsApsln |
| 043 | | Gpsln(5m)CpslnTpsln(5m)C |
| ASO- | 5583 | lnTpslnTpslnApsGpsGps(8nh)Gps(5m)CpsTps(5m)CpsTpsTps(5m)Cpsln |
| 044 | | (5m)CpslnApslnTpslnA |
| ASO- | 5584 | lnApslnTpsln(5m)CpslnAps(5m)CpsTps(5oh)CpsTpsTpsApsGpsGpsGps |
| 045 | | lnApslnApslnT |
| ASO- | 5585 | ln(5m)CpslnApslnTpsln(5m)CpsAps(5m)Cps(2s)Tps(5m)CpsTpsTpsAps |
| 046 | | GpsGpslnGpslnApslnA |
| ASO- | 5586 | ln(5m)Cpsln(5m)CpslnApslnTps(5m)CpsAps(5oh)CpsTps(5m)CpsTpsT |
| 047 | | psApsGpslnGpslnGpslnA |
| ASO- | 5587 | lnGpslnApsln(5m)CpslnGpsGpsGps(5oh)CpsTpsGps(5m)CpsAps(5m)Cps |
| 048 | | TpslnTpslnApsln(5m)C |
| ASO- | 5588 | lnTpslnGpsln(5m)CpslnAps(5m)CpsTps(8nh)ApsApsTpsGpsGps(5m)Cps |
| 049 | | ApslnTpslnApsln(5m)C |
| ASO- | 5589 | lnTpslnGpsln(5m)CpslnGpsApsGps(5oh)CpsTps(5m)CpsTpsApsTpsTps |
| 050 | | ln(5m)CpslnTpslnT |
| ASO- | 5590 | lnGpslnTpslnGpsln(5m)CpsGpsAps(8nh)Gps(5m)CpsTps(5m)CpsTpsAps |
| 051 | | TpslnTpsln(5m)CpslnT |
| ASO- | 5591 | ln(5m)CpslnApslnTpslnApsGpsTps(8nh)Aps(5m)CpsTpsAps(5m)CpsAps |
| 052 | | GpslnApslnTpslnA |
| ASO- | 5592 | lnTpslnTpslnApslnGps(5m)CpsAps(2s)TpsApsApsGps(5m)CpsApsGpsln |
| 053 | | TpslnTpslnG |
| ASO- | 5593 | lnApslnApsln(5m)CpslnApsTpsGps(2s)Tps(5m)CpsTpsGpsGpsAps(5m)C |
| 054 | | psln(5m)CpslnTpslnA |
| ASO- | 5594 | lnTpslnApslnTpsln(5m)CpsApsGps(8nh)Aps(5m)CpsApsTpsTpsApsTpsl |
| 055 | | nGpsln(5m)CpslnA |
| ASO- | 5595 | ln(5m)CpslnTpslnApslnTps(5m)CpsAps(8nh)GpsAps(5m)CpsApsTpsTps |
| 056 | | ApslnTpslnGpsln(5m)C |
| ASO- | 5596 | lnTpsln(5m)CpslnTpslnApsTps(5m)Cps(8nh)ApsGpsAps(5m)CpsApsTps |
| 057 | | TpslnApslnTpslnG |
| ASO- | 5597 | ln(5m)CpslnTpsln(5m)CpslnTpsApsTps(5oh)CpsApsGpsAps(5m)CpsAps |
| 058 | | TpslnTpslnApslnT |
| ASO- | 5598 | ln(5m)CpslnTpslnApslnTpsTpsAps(8nh)Aps(5m)CpsGpsTpsAps(5m)Cps |
| 059 | | (5m)CpslnTpslnGpslnT |
| ASO- | 5599 | lnGpslnTpslnApsln(5m)CpsGps(5m)Cps(8nh)Aps(5m)CpsAps(5m)CpsA |
| 060 | | psApsTpsln(5m)CpslnGpslnA |
| ASO- | 5600 | ln(5m)CpslnApslnGpslnTpsAps(5m)Cps(8nh)Gps(5m)CpsAps(5m)CpsA |
| 061 | | ps(5m)CpsApslnApslnTpsln(5m)C |
| ASO- | 5601 | lnGpsln(5m)Cpsln(5m)CpslnApsApsTps(2s)TpsTpsGpsGpsTps(5m)CpsA |
| 062 | | pslnTpsln(5m)CpslnT |
| ASO- | 5602 | lnApslnApslnTpslnTpsGpsTps(8nh)Gps(5m)CpsApsApsTpsTpsTpslnGps |
| 063 | | ln(5m)CpslnG |
| ASO- | 5603 | ln(5m)Cpsln(5m)CpslnApslnTpsApsTps(8nh)ApsGpsGps(5m)CpsApsGps |
| 064 | | (5m)CpslnTpsln(5m)CpslnT |
| ASO- | 5604 | lnTpsln(5m)Cpsln(5m)CpslnApsTpsAps(2s)TpsApsGpsGps(5m)CpsApsG |
| 065 | | psln(5m)CpslnTpsln(5m)C |
| ASO- | 5605 | lnTpslnTpslnApslnGpsGpsGps(5oh)CpsTps(5m)CpsTpsTps(5m)Cps(5m) |
| 066 | | CpslnApslnTpslnA |
| ASO- | 5606 | ln(5m)CpslnApslnTps(5m)Cps(8nh)Aps(5m)CpsTps(5m)CpsTpsTpsApsG |
| 067 | | psGpslnGpslnApslnA |
| ASO- | 5607 | lnGpslnTpslnGps(5m)Cps(8nh)GpsApsGps(5m)CpsTps(5m)CpsTpsApsT |
| 068 | | pslnTpsln(5m)CpslnT |
| ASO- | 5608 | lnGpslnTpslnGps(5m)Cps(8nh)GpsApsGps(5m)CpsTps(5m)CpsTpsApsT |
| 069 | | pslnTpsln(5m)CpslnT |
| ASO- | 5609 | lnTpslnTpslnApsGps(8nh)GpsGps(5m)CpsTps(5m)CpsTpsTps(5m)Cps |
| 070 | | (5m)CpslnApslnTpslnA |
| ASO- | 5610 | ln(5m)Cpsln(5m)CpslnApsTps(5oh)CpsAps(5m)CpsTps(5m)CpsTpsTpsA |
| 071 | | psGpslnGpslnGpslnA |
| ASO- | 5611 | lnTpslnGpsln(5m)CpsAps(5oh)CpsTpsApsApsTpsGpsGps(5m)CpsApslnT |
| 072 | | pslnApsln(5m)C |
| ASO- | 5612 | lnTpslnTpslnApsGps(5oh)CpsApsTpsApsApsGps(5m)CpsApsGpslnTpsln |
| 073 | | TpslnG |
| ASO- | 5613 | ln(5m)CpslnTpslnApsTps(5oh)CpsApsGpsAps(5m)CpsApsTpsTpsApslnT |
| 074 | | pslnGpsln(5m)C |
| ASO- | 5614 | ln(5m)CpslnTpslnApsTps(2s)TpsApsAps(5m)CpsGpsTpsAps(5m)Cps |
| 075 | | (5m)CpslnTpslnGpslnT |
| ASO- | 5615 | lnGpsln(5m)Cpsln(5m)CpsAps(8nh)ApsTpsTpsTpsGpsGpsTps(5m)CpsA |
| 076 | | pslnTpsln(5m)CpslnT |
| ASO- | 5616 | lnTpsln(5m)Cpsln(5m)CpsAps(2s)TpsApsTpsApsGpsGps(5m)CpsApsGps |
| 077 | | ln(5m)CpslnTpsln(5m)C |
| ASO- | 5617 | lnApslnTpsln(5m)CpsAps(5oh)CpsTps(5m)CpsTpsTpsApsGpsGpsGpslnA |
| 078 | | pslnApslnT |
| ASO- | 5618 | lnGpslnApsln(5m)CpsGps(8nh)GpsGps(5m)CpsTpsGps(5m)CpsAps(5m)C |
| 079 | | psTpslnTpslnApsln(5m)C |
| ASO- | 5619 | lnTpslnGpsln(5m)CpsGps(8nh)ApsGps(5m)CpsTps(5m)CpsTpsApsTpsT |
| 080 | | psln(5m)CpslnTpslnT |
| ASO- | 5620 | ln(5m)CpslnApslnTpsAps(8nh)GpsTpsAps(5m)CpsTpsAps(5m)CpsApsG |
| 081 | | pslnApslnTpslnA |
| ASO- | 5621 | lnApslnApsln(5m)CpsAps(2s)TpsGpsTps(5m)CpsTpsGpsGpsAps(5m)Cps |
| 082 | | ln(5m)CpslnTpslnA |
| ASO- | 5622 | lnTpslnApslnTps(5m)Cps(8nh)ApsGpsAps(5m)CpsApsTpsTpsApsTpslnG |
| 083 | | psln(5m)CpslnA |
| ASO- | 5623 | lnTpsln(5m)CpslnTpsAps(2s)Tps(5m)CpsApsGpsAps(5m)CpsApsTpsTps |
| 084 | | lnApslnTpslnG |
| ASO- | 5624 | ln(5m)CpslnTpsln(5m)CpsTps(8nh)ApsTps(5m)CpsApsGpsAps(5m)CpsA |
| 085 | | psTpslnTpslnApslnT |
| ASO- | 5625 | lnGpslnTpslnAps(5m)Cps(8nh)Gps(5m)CpsAps(5m)CpsAps(5m)CpsApsA |
| 086 | | psTpsln(5m)CpslnGpslnA |
| ASO- | 5626 | ln(5m)CpslnApslnGpsTps(8nh)Aps(5m)CpsGps(5m)CpsAps(5m)CpsAps |
| 087 | | (5m)CpsApslnApslnTpsln(5m)C |
| ASO- | 5627 | lnApslnApslnTpsTps(8nh)GpsTpsGps(5m)CpsApsApsTpsTpsTpslnGpsln |
| 088 | | (5m)CpslnG |
| ASO- | 5628 | ln(5m)Cpsln(5m)CpslnApsTps(8nh)ApsTpsApsGpsGps(5m)CpsApsGps |
| 089 | | (5m)CpslnTpsln(5m)CpslnT |
| ASO- | 5629 | lnTpslnTpslnApsTps(5oh)CpsAps(5m)Cps(5m)CpsGpsTpsTps(5m)CpsT |
| 090 | | psln(5m)CpslnApslnT |
| ASO- | 5630 | lnApslnApslnTps(5m)Cps(2s)Tps(5m)Cps(5m)Cps(5m)CpsApsApsGpsA |
| 091 | | psGpslnApslnGpslnA |
| ASO- | 5631 | lnApslnApslnApsAps(2s)TpsApsTpsAps(5m)CpsTpsTpsTps(5m)CpslnT |
| 092 | | psln(5m)CpslnT |
| ASO- | 5632 | lnTpslnApslnTpsTps(8nh)Aps(5m)CpsGpsApsTpsApsTpsGpsTpslnGpsln |
| 093 | | GpslnA |
| ASO- | 5633 | lnApsln(5m)CpslnApsAps(8nh)Gps(5m)CpsApsTps(5m)CpsTpsTps(5m)C |
| 094 | | psTpslnApslnApslnT |
| ASO- | 5634 | ln(5m)CpslnTpslnApsTps(5m)Cps(8nh)ApsGpsAps(5m)CpsApsTpsTpsA |
| 095 | | pslnTpslnGpsln(5m)C |
| ASO- | 5635 | lnTpsln(5m)CpslnTpsApsTps(5oh)CpsApsGpsAps(5m)CpsApsTpsTpslnA |
| 096 | | pslnTpslnG |
| ASO- | 5636 | ln(5m)CpslnTpsln(5m)CpsTpsAps(2s)Tps(5m)CpsApsGpsAps(5m)CpsA |
| 097 | | psTpslnTpslnApslnT |
| ASO- | 5637 | ln(5m)CpslnTpslnApsTpsTps(8nh)ApsAps(5m)CpsGpsTpsAps(5m)Cps |
| 098 | | (5m)CpslnTpslnGpslnT |
| ASO- | 5638 | lnGpslnTpslnAps(5m)CpsGps(5oh)CpsAps(5m)CpsAps(5m)CpsApsApsT |
| 099 | | psln(5m)CpslnGpslnA |
| ASO- | 5639 | ln(5m)CpslnApslnGpsTpsAps(5oh)CpsGps(5m)CpsAps(5m)CpsAps(5m)C |
| 100 | | psApslnApslnTpsln(5m)C |
| ASO- | 5640 | lnGpsln(5m)Cpsln(5m)CpsApsAps(2s)TpsTpsTpsGpsGpsTps(5m)CpsAps |
| 101 | | lnTpsln(5m)CpslnT |
| ASO- | 5641 | lnApslnApslnTpsTpsGps(2s)TpsGps(5m)CpsApsApsTpsTpsTpslnGpsln |
| 102 | | (5m)CpslnG |
| ASO- | 5642 | ln(5m)Cpsln(5m)CpslnApsTpsAps(2s)TpsApsGpsGps(5m)CpsApsGps |
| 103 | | (5m)CpslnTpsln(5m)CpslnT |
| ASO- | 5643 | lnTpsln(5m)Cpsln(5m)CpsApsTps(8nh)ApsTpsApsGpsGps(5m)CpsApsG |
| 104 | | psln(5m)CpslnTpsln(5m)C |
| ASO- | 5644 | lnTpslnTpslnApsGpsGps(8nh)Gps(5m)CpsTps(5m)CpsTpsTps(5m)Cps |
| 105 | | (5m)CpslnApslnTpslnA |
| ASO- | 5645 | lnApslnTpsln(5m)CpsAps(5m)Cps(2s)Tps(5m)CpsTpsTpsApsGpsGpsGps |
| 106 | | lnApslnApslnT |
| ASO- | 5646 | ln(5m)CpslnApslnTps(5m)CpsAps(5oh)CpsTps(5m)CpsTpsTpsApsGpsG |
| 107 | | pslnGpslnApslnA |
| ASO- | 5647 | ln(5m)Cpsln(5m)CpslnApsTps(5m)Cps(8nh)Aps(5m)CpsTps(5m)CpsTps |
| 108 | | TpsApsGpslnGpslnGpslnA |
| ASO- | 5648 | lnGpslnApsln(5m)CpsGpsGps(8nh)Gps(5m)CpsTpsGps(5m)CpsAps(5m)C |
| 109 | | psTpslnTpslnApsln(5m)C |
| ASO- | 5649 | lnTpslnGpsln(5m)CpsAps(5m)Cps(2s)TpsApsApsTpsGpsGps(5m)CpsAps |
| 110 | | lnTpslnApsln(5m)C |
| ASO- | 5650 | lnTpslnGpsln(5m)CpsGpsAps(8nh)Gps(5m)CpsTps(5m)CpsTpsApsTpsT |
| 111 | | psln(5m)CpslnTpslnT |
| ASO- | 5651 | lnGpslnTpslnGps(5m)CpsGps(8nh)ApsGps(5m)CpsTps(5m)CpsTpsApsT |
| 112 | | pslnTpsln(5m)CpslnT |
| ASO- | 5652 | ln(5m)CpslnApslnTpsApsGps(2s)TpsAps(5m)CpsTpsAps(5m)CpsApsGps |
| 113 | | lnApslnTpslnA |
| ASO- | 5653 | lnTpslnTpslnApsGps(5m)Cps(8nh)ApsTpsApsApsGps(5m)CpsApsGpslnT |
| 114 | | pslnTpslnG |
| ASO- | 5654 | lnApslnApsln(5m)CpsApsTps(8nh)GpsTps(5m)CpsTpsGpsGpsAps(5m)C |
| 115 | | psln(5m)CpslnTpslnA |
| ASO- | 5655 | lnTpslnApslnTps(5m)CpsAps(8nh)GpsAps(5m)CpsApsTpsTpsApsTpslnG |
| 116 | | psln(5m)CpslnA |
| ASO- | 5656 | lnApslnGpslnTpslnApsTpsAps(5m)CpsTpsTpsApsGps(5m)CpsApsApsln |
| 117 | | (5m)CpslnApslnTpslnT |
| ASO- | 5657 | lnTpslnApslnGpslnGpsApsTpsAps(5m)CpsApsAps(5m)CpsAps(5m)CpsG |
| 118 | | pslnTpsln(5m)Cpsln(5m)Cpsln(5m)C |
| ASO- | 5658 | lnApslnApslnTpslnApsTpsApsApsAps(5m)CpsAps(5m)Cps(5m)CpsAps |
| 119 | | (5m)CpslnTpslnApslnTpslnG |
| ASO- | 5659 | ln(5m)CpslnTpslnApsTps(2s)TpsApsAps(5m)CpsGpsTpsAps(5m)Cpsln |
| 120 | | (5m)CpslnTpslnGpslnT |
| ASO- | 5660 | lnGpslnTpslnAps(5m)Cps(8nh)Gps(5m)CpsAps(5m)CpsAps(5m)CpsApsA |
| 121 | | pslnTpsln(5m)CpslnGpslnA |
| ASO- | 5661 | ln(5m)CpslnApslnGpsTps(8nh)Aps(5m)CpsGps(5m)CpsAps(5m)CpsAps |
| 122 | | (5m)CpslnApslnApslnTpsln(5m)C |
| ASO- | 5662 | lnGpsln(5m)Cpsln(5m)CpsAps(8nh)ApsTpsTpsTpsGpsGpsTps(5m)Cpsln |
| 123 | | ApslnTpsln(5m)CpslnT |
| ASO- | 5663 | lnApslnApslnTpsTps(8nh)GpsTpsGps(5m)CpsApsApsTpsTpslnTpslnGps |
| 124 | | ln(5m)CpslnG |
| ASO- | 5664 | ln(5m)Cpsln(5m)CpslnApsTps(8nh)ApsTpsApsGpsGps(5m)CpsApsGpsln |
| 125 | | (5m)CpslnTpsln(5m)CpslnT |
| ASO- | 5665 | lnTpsln(5m)Cpsln(5m)CpsAps(2s)TpsApsTpsApsGpsGps(5m)CpsApslnG |
| 126 | | psln(5m)CpslnTpsln(5m)C |
| ASO- | 5666 | lnTpslnTpslnApsGps(8nh)GpsGps(5m)CpsTps(5m)CpsTpsTps(5m)Cpsln |
| 127 | | (5m)CpslnApslnTpslnA |
| ASO- | 5667 | lnApslnTpsln(5m)CpsAps(5oh)CpsTps(5m)CpsTpsTpsApsGpsGpslnGps |
| 128 | | lnApslnApslnT |
| ASO- | 5668 | lnApslnTpsln(5m)CpsAps(5oh)CpsTps(5m)CpsTpsTpsApsGpsGpslnGps |
| 129 | | lnApslnApslnT |
| ASO- | 5669 | lnTpslnGpsln(5m)CpsAps(5oh)CpsTpsApsApsTpsGpsGps(5m)CpslnAps |
| 130 | | lnTpslnApsln(5m)C |
| ASO- | 5670 | lnTpslnTpslnApsGps(5oh)CpsApsTpsApsApsGps(5m)CpsApslnGpslnTps |
| 131 | | lnTpslnG |
| ASO- | 5671 | ln(5m)CpslnTpslnApsTps(5oh)CpsApsGpsAps(5m)CpsApsTpsTpslnAps |
| 132 | | lnTpslnGpsln(5m)C |
| ASO- | 5672 | ln(5m)CpslnApslnTps(5m)Cps(8nh)Aps(5m)CpsTps(5m)CpsTpsTpsApsG |
| 133 | | pslnGpslnGpslnApslnA |
| ASO- | 5673 | ln(5m)Cpsln(5m)CpslnApsTps(5oh)CpsAps(5m)CpsTps(5m)CpsTpsTpsA |
| 134 | | pslnGpslnGpslnGpslnA |
| ASO- | 5674 | lnGpslnApsln(5m)CpsGps(8nh)GpsGps(5m)CpsTpsGps(5m)CpsAps(5m)C |
| 135 | | pslnTpslnTpslnApsln(5m)C |
| ASO- | 5675 | lnTpslnGpsln(5m)CpsGps(8nh)ApsGps(5m)CpsTps(5m)CpsTpsApsTpsln |
| 136 | | Tpsln(5m)CpslnTpslnT |
| ASO- | 5676 | lnGpslnTpslnGps(5m)Cps(8nh)GpsApsGps(5m)CpsTps(5m)CpsTpsApsln |
| 137 | | TpslnTpsln(5m)CpslnT |
| ASO- | 5677 | ln(5m)CpslnApslnTpsAps(8nh)GpsTpsAps(5m)CpsTpsAps(5m)CpsApsln |
| 138 | | GpslnApslnTpslnA |
| ASO- | 5678 | lnApslnApsln(5m)CpsAps(2s)TpsGpsTps(5m)CpsTpsGpsGpsApsln(5m)C |
| 139 | | psln(5m)CpslnTpslnA |
| ASO- | 5679 | lnTpslnApslnTps(5m)Cps(8nh)ApsGpsAps(5m)CpsApsTpsTpsApslnTps |
| 140 | | lnGpsln(5m)CpslnA |
| ASO- | 5680 | lnTpsln(5m)CpslnTpsAps(2s)Tps(5m)CpsApsGpsAps(5m)CpsApsTpslnT |
| 141 | | pslnApslnTpslnG |
| ASO- | 5681 | ln(5m)CpslnTpsln(5m)CpsTps(8nh)ApsTps(5m)CpsApsGpsAps(5m)CpsA |
| 142 | | pslnTpslnTpslnApslnT |
| ASO- | 5682 | moe(5m)Cpsmoe(5m)CpsmoeApsmoeTpsmoe(5m)CpsAps(5m)CpsTps(5m)C |
| 143 | | psTpsTpsApsGpsGpsGpsmoeApsmoeApsmoeTpsmoe(5m)CpsmoeT |
| ASO- | 5683 | moeTpsmoe(5m)Cpsmoe(5m)CpsmoeApsmoeTps(5m)CpsAps(5m)CpsTps |
| 144 | | (5m)CpsTpsTpsApsGpsGpsmoeGpsmoeApsmoeApsmoeTpsmoe(5m)C |
| ASO- | 5684 | moeTpsmoeTpsmoe(5m)Cpsmoe(5m)CpsmoeApsTps(5m)CpsAps(5m)CpsTps |
| 145 | | (5m)CpsTpsTpsApsGpsmoeGpsmoeGpsmoeApsmoeApsmoeT |
| ASO- | 5685 | moeGpsmoeTpsmoeTpsmoe(5m)Cpsmoe(5m)CpsApsTps(5m)CpsAps(5m)Cps |
| 146 | | Tps(5m)CpsTpsTpsApsmoeGpsmoeGpsmoeGpsmoeApsmoeA |
| ASO- | 5686 | moeApsmoeGpsmoeTpsmoeTpsmoe(5m)Cps(5m)CpsApsTps(5m)CpsAps(5m) |
| 147 | | CpsTps(5m)CpsTpsTpsmoeApsmoeGpsmoeGpsmoeGpsmoeA |
| ASO- | 5687 | moeGpsmoeApsmoe(5m)CpsmoeGpsmoeGpsGps(5m)CpsTpsGps(5m)CpsAps |
| 148 | | (5m)CpsTpsTpsApsmoe(5m)CpsmoeApsmoe(5m)Cpsmoe(5m)CpsmoeG |
| ASO- | 5688 | moeApsmoeGpsmoeApsmoe(5m)CpsmoeGpsGpsGps(5m)CpsTpsGps(5m)CpsA |
| 149 | | ps(5m)CpsTpsTpsmoeApsmoe(5m)CpsmoeApsmoe(5m)Cpsmoe(5m)C |
| ASO- | 5689 | moeApsmoeApsmoeGpsmoeApsmoe(5m)CpsGpsGpsGps(5m)CpsTpsGps(5m)C |
| 150 | | psAps(5m)CpsTpsmoeTpsmoeApsmoe(5m)CpsmoeApsmoe(5m)C |
| ASO- | 5690 | moeTpsmoeApsmoeApsmoeGpsmoeAps(5m)CpsGpsGpsGps(5m)CpsTpsGps |
| 151 | | (5m)CpsAps(5m)CpsmoeTpsmoeTpsmoeApsmoe(5m)CpsmoeA |
| ASO- | 5691 | moeGpsmoeTpsmoeApsmoeApsmoeGpsAps(5m)CpsGpsGpsGps(5m)CpsTpsG |
| 152 | | ps(5m)CpsApsmoe(5m)CpsmoeTpsmoeTpsmoeApsmoe(5m)C |
| ASO- | 5692 | moeTpsmoeGpsmoe(5m)CpsmoeGpsmoeApsGps(5m)CpsTps(5m)CpsTpsApsT |
| 153 | | psTps(5m)CpsTpsmoeTpsmoeTpsmoeGpsmoe(5m)CpsmoeA |
| ASO- | 5693 | moeGpsmoeTpsmoeGpsmoe(5m)CpsmoeGpsApsGps(5m)CpsTps(5m)CpsTpsA |
| 154 | | psTpsTps(5m)CpsmoeTpsmoeTpsmoeTpsmoeGpsmoe(5m)C |
| ASO- | 5694 | moeGpsmoeGpsmoeTpsmoeGpsmoe(5m)CpsGpsApsGps(5m)CpsTps(5m)CpsT |
| 155 | | psApsTpsTpsmoe(5m)CpsmoeTpsmoeTpsmoeTpsmoeG |
| ASO- | 5695 | moe(5m)CpsmoeGpsmoeGpsmoeTpsmoeGps(5m)CpsGpsApsGps(5m)CpsTps |
| 156 | | (5m)CpsTpsApsTpsmoeTpsmoe(5m)CpsmoeTpsmoeTpsmoeT |
| ASO- | 5696 | moeApsmoe(5m)CpsmoeGpsmoeGpsmoeTpsGps(5m)CpsGpsApsGps(5m)CpsT |
| 157 | | ps(5m)CpsTpsApsmoeTpsmoeTpsmoe(5m)CpsmoeTpsmoeT |
| ASO- | 5697 | moeApsmoeApsmoe(5m)CpsmoeApsmoeTpsGpsTps(5m)CpsTpsGpsGpsAps |
| 158 | | (5m)Cps(5m)CpsTpsmoeApsmoeTpsmoeApsmoeGpsmoeT |
| ASO- | 5698 | moeGpsmoeApsmoeApsmoe(5m)CpsmoeApsTpsGpsTps(5m)CpsTpsGpsGpsA |
| 159 | | ps(5m)Cps(5m)CpsmoeTpsmoeApsmoeTpsmoeApsmoeG |
| ASO- | 5699 | moeGpsmoeGpsmoeApsmoeApsmoe(5m)CpsApsTpsGpsTps(5m)CpsTpsGpsG |
| 160 | | psAps(5m)Cpsmoe(5m)CpsmoeTpsmoeApsmoeTpsmoeA |
| ASO- | 5700 | moeApsmoeGpsmoeGpsmoeApsmoeAps(5m)CpsApsTpsGpsTps(5m)CpsTpsG |
| 161 | | psGpsApsmoe(5m)Cpsmoe(5m)CpsmoeTpsmoeApsmoeT |
| ASO- | 5701 | moeGpsmoeApsmoeGpsmoeGpsmoeApsAps(5m)CpsApsTpsGpsTps(5m)CpsT |
| 162 | | psGpsGpsmoeApsmoe(5m)Cpsmoe(5m)CpsmoeTpsmoeA |
| ASO- | 5702 | moe(5m)Cpsmoe(5m)CpsmoeApsmoeTpsmoe(5m)CpsAps(5m)Cps(2s)Tps |
| 163 | | (5m)CpsTpsTpsApsGpsGpsGpsmoeApsmoeApsmoeTpsmoe(5m)CpsmoeT |
| ASO- | 5703 | moeTpsmoe(5m)Cpsmoe(5m)CpsmoeApsmoeTps(5m)CpsAps(5oh)CpsTps |
| 164 | | (5m)CpsTpsTpsApsGpsGpsmoeGpsmoeApsmoeApsmoeTpsmoe(5m)C |
| ASO- | 5704 | moeTpsmoeTpsmoe(5m)Cpsmoe(5m)CpsmoeApsTps(5m)Cps(8nh)Aps(5m)C |
| 165 | | psTps(5m)CpsTpsTpsApsGpsmoeGpsmoeGpsmoeApsmoeApsmoeT |
| ASO- | 5705 | moeGpsmoeTpsmoeTpsmoe(5m)Cpsmoe(5m)CpsApsTps(5oh)CpsAps(5m)C |
| 166 | | psTps(5m)CpsTpsTpsApsmoeGpsmoeGpsmoeGpsmoeApsmoeA |
| ASO- | 5706 | moeApsmoeGpsmoeTpsmoeTpsmoe(5m)Cps(5m)CpsAps(2s)Tps(5m)CpsAps |
| 167 | | (5m)CpsTps(5m)CpsTpsTpsmoeApsmoeGpsmoeGpsmoeGpsmoeA |
| ASO- | 5707 | moeGpsmoeApsmoe(5m)CpsmoeGpsmoeGpsGps(5m)Cps(2s)TpsGps(5m)Cps |
| 168 | | Aps(5m)CpsTpsTpsApsmoe(5m)CpsmoeApsmoe(5m)Cpsmoe(5m)CpsmoeG |
| ASO- | 5708 | moeApsmoeGpsmoeApsmoe(5m)CpsmoeGpsGpsGps(5oh)CpsTpsGps(5m)Cps |
| 169 | | Aps(5m)CpsTpsTpsmoeApsmoe(5m)CpsmoeApsmoe(5m)Cpsmoe(5m)C |
| ASO- | 5709 | moeApsmoeApsmoeGpsmoeApsmoe(5m)CpsGpsGps(8nh)Gps(5m)CpsTpsGps |
| 170 | | (5m)CpsAps(5m)CpsTpsmoeTpsmoeApsmoe(5m)CpsmoeApsmoe(5m)C |
| ASO- | 5710 | moeTpsmoeApsmoeApsmoeGpsmoeAps(5m)CpsGps(8nh)GpsGps(5m)CpsTps |
| 171 | | Gps(5m)CpsAps(5m)CpsmoeTpsmoeTpsmoeApsmoe(5m)CpsmoeA |
| ASO- | 5711 | moeGpsmoeTpsmoeApsmoeApsmoeGpsAps(5m)Cps(8nh)GpsGpsGps(5m)Cps |
| 172 | | TpsGps(5m)CpsApsmoe(5m)CpsmoeTpsmoeTpsmoeApsmoe(5m)C |
| ASO- | 5712 | moeTpsmoeGpsmoe(5m)CpsmoeGpsmoeApsGps(5m)Cps(2s)Tps(5m)CpsTps |
| 173 | | ApsTpsTps(5m)CpsTpsmoeTpsmoeTpsmoeGpsmoe(5m)CpsmoeA |
| ASO- | 5713 | moeGpsmoeTpsmoeGpsmoe(5m)CpsmoeGpsApsGps(5oh)CpsTps(5m)CpsTps |
| 174 | | ApsTpsTps(5m)CpsmoeTpsmoeTpsmoeTpsmoeGpsmoe(5m)C |
| ASO- | 5714 | moeGpsmoeGpsmoeTpsmoeGpsmoe(5m)CpsGpsAps(8nh)Gps(5m)CpsTps |
| 175 | | (5m)CpsTpsApsTpsTpsmoe(5m)CpsmoeTpsmoeTpsmoeTpsmoeG |
| ASO- | 5715 | moe(5m)CpsmoeGpsmoeGpsmoeTpsmoeGps(5m)CpsGps(8nh)ApsGps(5m)C |
| 176 | | psTps(5m)CpsTpsApsTpsmoeTpsmoe(5m)CpsmoeTpsmoeTpsmoeT |
| ASO- | 5716 | moeApsmoeApsmoe(5m)CpsmoeApsmoeTpsGpsTps(5oh)CpsTpsGpsGpsAps |
| 177 | | (5m)Cps(5m)CpsTpsmoeApsmoeTpsmoeApsmoeGpsmoeT |
| ASO- | 5717 | moeGpsmoeApsmoeApsmoe(5m)CpsmoeApsTpsGps(2s)Tps(5m)CpsTpsGpsG |
| 178 | | psAps(5m)Cps(5m)CpsmoeTpsmoeApsmoeTpsmoeApsmoeG |
| ASO- | 5718 | moeGpsmoeGpsmoeApsmoeApsmoe(5m)CpsApsTps(8nh)GpsTps(5m)CpsTps |
| 179 | | GpsGpsAps(5m)Cpsmoe(5m)CpsmoeTpsmoeApsmoeTpsmoeA |

lnX = locked nucleic acid (LNA) (e.g., lnG = locked nucleic acid (LNA) G);
(5m)lnX = locked nucleic acid (LNA)-5methy1 nucleotide (e.g., (5m)lNC_= LNA-5methy1 C);
(5m)X = 5 methyl nucleotide (e.g., (5m)C = 5 methyl C);
mX = 2′-O-methoxy nucleotide (e.g., mA = 2′-O-methoxy A);
(8nh)X = 8-amino nucleotide (e.g., (8nh)G = 8-amino G);
(2s)X = 2-thio nucleotide (e.g., (2s)T = 2-thio T);
(5oh)X = 5-hydroxy nucleotide (e.g., (5oh)C = 5-hydroxy C);
cpX = scpX = cyclopropyl nucleotide (e.g., cp (5m) C = scp (5m) C = cyclopropyl (5m) C);
ps = phosphorothioate linkages;
moeX = 2′-O-methoxyethylribose nucleotide (e.g., moeG = 2′-O-methoxyethylribose G);
moe(5m)X = 2′-O-methoxyethylribose 5 methyl nucleotide (e.g., moe(5m)C = 2′-O-methoxyethylribose 5 methyl C).

In some embodiments, an ASO of the present disclosure may comprise any one of SEQ ID NOs: 5539-5718. In some embodiments, the ASO can be a 16-mer or a 20-mer.

In some embodiments, the ASO may be selected from ASO-143 (SEQ ID NO: 5682), ASO-144 (SEQ ID NO: 5683), ASO-145 (SEQ ID NO: 5684), ASO-146 (SEQ ID NO: 5685), ASO-147 (SEQ ID NO: 5686), ASO-148 (SEQ ID NO: 5687), ASO-149 (SEQ ID NO: 5688), ASO-150 (SEQ ID NO: 5689), ASO-151 (SEQ ID NO: 5690), ASO-152 (SEQ ID NO: 5691), ASO-153 (SEQ ID NO: 5692), ASO-154 (SEQ ID NO: 5693), ASO-155 (SEQ ID NO: 5694), ASO-156 (SEQ ID NO: 5695), ASO-158 (SEQ ID NO: 5697), ASO-159 (SEQ ID NO: 5698), and ASO-160 (SEQ ID NO: 5699), or a further modified form thereof. For example, ASO-163 to ASO-179 correspond to each of ASO-143 to ASO-156/ASO-158 to ASO-160, respectively, but ASO-163 to ASO-179 have been further modified at position 3 to detoxify the ASOs that were active but showed some toxicity. Such detoxifying modification can include, but are not limited to, incorporation of scpBNA, AmNA (N—H), AmNA (N-Me), GuNA, GuNA (N-R) where R is selected from Me, Et, iPr, tBu and combinations thereof. Accordingly, in some embodiments, the ASO may be selected from ASO-163 (SEQ ID NO: 5702), ASO-164 (SEQ ID NO: 5703), ASO-165 (SEQ ID NO: 5704), ASO-166 (SEQ ID NO: 5705), ASO-167 (SEQ ID NO: 5706), ASO-168 (SEQ ID NO: 5707), ASO-169 (SEQ ID NO: 5708), ASO-170 (SEQ ID NO: 5709), ASO-171 (SEQ ID NO: 5710), ASO-172 (SEQ ID NO: 5711), ASO-173 (SEQ ID NO: 5712), ASO-174 (SEQ ID NO: 5713), ASO-175 (SEQ ID NO: 5714), ASO-176 (SEQ ID NO: 5715), ASO-177 (SEQ ID NO: 5716), ASO-178 (SEQ ID NO: 5717), and ASO-179 (SEQ ID NO: 5718). All of these siNA also showed high potency across screening assays (see Examples). Those skilled in the art will understand that other analogs can be similarly constructed.

Any of the foregoing specific embodiments can be incorporated into a pharmaceutical composition, either alone or in combination with 1, 2, 3, 4, 5, 6, 7, 8, 9, or 10 or more additional siNA disclosed herein. Any of the foregoing specific embodiments can be used to treat or prevent viral infections, such as coronavirus infections (e.g., COVID-19) pursuant to the methods and uses disclosed herein.

### Pharmaceutical Compositions

The present disclosure also encompasses pharmaceutical compositions comprising ASOs of the present disclosure. One embodiment is a pharmaceutical composition comprising one or more ASO of the present disclosure, and a pharmaceutically acceptable diluent or carrier.

In some embodiments, the pharmaceutical compositions comprising any of the ASO molecules, sense strands, antisense strands, first nucleotide sequences, or second nucleotide sequences described herein. The compositions may comprise 1, 2, 3, 4, 5, 6, 7, 8, 9, 10, 11, 12 or more ASO molecules described herein. The compositions may comprise a first nucleotide sequence comprising a nucleotide sequence of any one SEQ ID NOs: 1-5534. In some embodiments, the composition comprises a second nucleotide sequence comprising a nucleotide sequence of any one of SEQ ID NOs: 1-5534.

In some embodiments, the pharmaceutical composition containing the ASO of the present disclosure is formulated for systemic administration via parenteral delivery. Parenteral administration includes intravenous, intra-arterial, subcutaneous, intraperitoneal or intramuscular injection or infusion; also subdermal administration, e.g., via an implanted device. In a preferred embodiment, the pharmaceutical composition containing the ASO of the present disclosure is formulated for subcutaneous (SC) or intravenous (IV) delivery. Formulations for parenteral administration may include sterile aqueous solutions, which may also contain buffers, diluents and other pharmaceutically acceptable additives as understood by the skilled artisan. For intravenous use, the total concentration of solutes may be controlled to render the preparation isotonic.

The pharmaceutical compositions containing the ASO of the present disclosure are useful for treating a disease or disorder, e.g., associated with the expression or activity of a coronavirus gene, more specifically a non-structural protein, such as nsp8, nsp9, nsp10, nsp11, nsp12, nsp13, nsp14, or nsp15.

In some embodiments, the pharmaceutical composition comprises an ASO of the present disclosure that is complementary or hybridizes to a viral target RNA sequence (e.g., a non-structural protein of coronavirus), and a pharmaceutically acceptable diluent or carrier. When the pharmaceutical composition comprises two or more ASOs, the ASOs may be present in varying amounts. For example, in some embodiments, the weight ratio of first ASO to second ASO is 1:4 to 4:1, e.g., 1:4, 1:3, 1:2, 1:1, 2:1, 3:1, or 4:1. In some embodiments, the molar ratio of first ASO to second ASO is 1:4 to 4:1, e.g., 1:4, 1:3, 1:2, 1:1, 2:1, 3:1, or 4:1.

In some embodiments, the pharmaceutical composition comprises an amount of one or more of the ASO molecules described herein formulated with one or more pharmaceutically acceptable carriers (additives) and/or diluents. The pharmaceutical compositions may be specially formulated for administration in solid or liquid form, including those adapted for the following: (1) parenteral administration, for example, by subcutaneous, intramuscular, intravenous or epidural injection as, for example, a sterile solution or suspension, or sustained-release formulation; (2) topical application, for example, as a cream, ointment, or a controlled-release patch or spray applied to the skin; (3) intravaginally or intrarectally, for example, as a pessary, cream or foam; (4) sublingually; (5) ocularly; (6) transdermally; or (7) nasally.

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Examples of pharmaceutically-acceptable antioxidants include: (1) water soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal chelating agents, such as citric acid, ethylenediamine tetraacetic acid (EDTA), sorbitol, tartaric acid, phosphoric acid, and the like.

Formulations of the present disclosure include those suitable for nasal, topical (including buccal and sublingual), rectal, vaginal and/or parenteral administration. The formulations may be presented in unit dosage form and may be prepared by any methods well known in the art of pharmacy. The amount of the one or more ASOs which can be combined with a carrier material to produce a single dosage form will vary depending upon the host being treated, the particular mode of administration, and/or the type and severity of coronavirus infection. The amount of active ingredient which can be combined with a carrier material to produce a single dosage form will generally be that amount of the compound (e.g., ASO molecule) which produces a therapeutic effect. Generally, out of one hundred percent, this amount will range from about 0.1 percent to about ninety-nine percent of active ingredient, preferably from about 5 percent to about 70 percent, most preferably from about 10 percent to about 30 percent.

In some embodiments, a formulation of the present disclosure comprises an excipient selected from the group consisting of cyclodextrins, celluloses, liposomes, micelle forming agents, e.g., bile acids, and polymeric carriers, e.g., polyesters and polyanhydrides; and a compound (e.g., ASO molecule) of the present disclosure.

Methods of preparing these formulations or compositions include the step of bringing into association a compound (e.g., ASO molecule) of the present disclosure with the carrier and, optionally, one or more accessory ingredients. In general, the formulations are prepared by uniformly and intimately bringing into association a compound (e.g., ASO molecule) of the present disclosure with liquid carriers, or finely divided solid carriers, or both, and then, if necessary, shaping the product.

Formulations of the disclosure suitable for a suspension in an aqueous or non-aqueous liquid, or as an oil-in-water or water-in-oil liquid emulsion, or as an elixir or syrup, each containing a predetermined amount of a compound (e.g., ASO molecule) of the present disclosure as an active ingredient. A compound (e.g., ASO molecule) of the present disclosure may also be administered as a bolus, electuary, or paste.

In dosage forms of the disclosure, the active ingredient may be mixed with one or more pharmaceutically-acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds and surfactants, such as poloxamer and sodium lauryl sulfate; (7) wetting agents, such as, for example, cetyl alcohol, glycerol monostearate, and non-ionic surfactants; (8) absorbents, such as kaolin and bentonite clay; (9) lubricants, such as talc, calcium stearate, magnesium stearate, solid polyethylene glycols, sodium lauryl sulfate, zinc stearate, sodium stearate, stearic acid, and mixtures thereof; (10) coloring agents; and (11) controlled release agents such as crospovidone or ethyl cellulose.

The disclosed dosage forms may be sterilized by, for example, filtration through a bacteria-retaining filter, or by incorporating sterilizing agents in the form of sterile solid compositions which can be dissolved in sterile water, or some other sterile injectable medium immediately before use. These compositions may also optionally contain opacifying agents and may be of a composition that they release the active ingredient(s) only, or preferentially, in a certain portion of the gastrointestinal tract, optionally, in a delayed manner. Examples of embedding compositions which can be used include polymeric substances and waxes. The active ingredient can also be in micro-encapsulated form, if appropriate, with one or more of the above-described excipients.

Liquid dosage forms the compounds (e.g., ASO molecules) of the disclosure include pharmaceutically acceptable emulsions, microemulsions, solutions, suspensions, syrups and elixirs. In addition to the active ingredient, the liquid dosage forms may contain inert diluents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (I particular, cottonseed, groundnut, corn, germ, olive, castor and sesame oils), glycerol, tetrahydrofuryl alcohol, polyethylene glycols and fatty acid esters of sorbitan, and mixtures thereof.

Besides inert diluents, the compositions can also include adjuvants such as wetting agents, emulsifying and suspending agents, sweetening, flavoring, coloring, perfuming and preservative agents.

Suspensions, in addition to the active compounds (e.g., ASO molecules), may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Formulations of the pharmaceutical compositions of the disclosure for rectal or vaginal administration may be presented as a suppository, which may be prepared by mixing one or more compounds (e.g., ASO molecules) of the disclosure with one or more suitable nonirritating excipients or carriers comprising, for example, cocoa butter, polyethylene glycol, a suppository wax or a salicylate, and which is solid at room temperature, but liquid at body temperature and, therefore, will melt in the rectum or vaginal cavity and release the active compound (e.g., ASO molecule).

Formulations of the present disclosure which are suitable for vaginal administration also include pessaries, tampons, creams, gels, pastes, foams or spray formulations containing such carriers as are known in the art to be appropriate.

Dosage forms for the topical or transdermal administration of a compound (e.g., ASO molecule) of this disclosure include powders, sprays, ointments, pastes, creams, lotions, gels, solutions, patches and inhalants. The active compound (e.g., ASO molecule) may be mixed under sterile conditions with a pharmaceutically acceptable carrier, and with any preservatives, buffers, or propellants which may be required.

The ointments, pastes, creams and gels may contain, in addition to an active compound (e.g., ASO molecule) of this disclosure, excipients, such as animal and vegetable fats, oils, waxes, paraffins, starch, tragacanth, cellulose derivatives, polyethylene glycols, silicones, bentonites, silicic acid, talc and zinc oxide, or mixtures thereof

Powders and sprays can contain, in addition to a compound (e.g., ASO molecule) of this disclosure, excipients such as lactose, talc, silicic acid, aluminum hydroxide, calcium silicates and polyamide powder, or mixtures of these substances. Sprays can additionally contain customary propellants, such as chlorofluorohydrocarbons and volatile unsubstituted hydrocarbons, such as butane and propane.

Transdermal patches have the added advantage of providing controlled delivery of a compound (e.g., ASO molecule) of the present disclosure to the body. Such dosage forms can be made by dissolving or dispersing the compound (e.g., ASO molecule) in the proper medium. Absorption enhancers can also be used to increase the flux of the compound (e.g., ASO molecule) across the skin. The rate of such flux can be controlled by either providing a rate controlling membrane or dispersing the compound (e.g., ASO molecule) in a polymer matrix or gel.

Ophthalmic formulations, eye ointments, powders, solutions and the like, are also contemplated as being within the scope of this invention.

Pharmaceutical compositions of this disclosure suitable for parenteral administration comprise one or more compounds (e.g., ASO molecules) of the disclosure in combination with one or more pharmaceutically-acceptable sterile isotonic aqueous or nonaqueous solutions, dispersions, suspensions or emulsions, or sterile powders which may be reconstituted into sterile injectable solutions or dispersions just prior to use, which may contain sugars, alcohols, antioxidants, buffers, bacteriostats, solutes which render the formulation isotonic with the blood of the intended recipient or suspending or thickening agents.

Examples of suitable aqueous and nonaqueous carriers which may be employed in the pharmaceutical compositions of the disclosure include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of the action of microorganisms upon the subject compounds may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents which delay absorption such as aluminum monostearate and gelatin.

In some cases, in order to prolong the effect of a drug, it is desirable to slow the absorption of the drug from subcutaneous or intramuscular injection. This may be accomplished by the use of a liquid suspension of crystalline or amorphous material having poor water solubility. The rate of absorption of the drug then depends upon its rate of dissolution which, in turn, may depend upon crystal size and crystalline form. Alternatively, delayed absorption of a parenterally-administered drug form is accomplished by dissolving or suspending the drug in an oil vehicle.

Injectable depot forms are made by forming microencapsule matrices of the subject compounds (e.g., ASO molecules) in biodegradable polymers such as polylactide-polyglycolide. Depending on the ratio of drug to polymer, and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions which are compatible with body tissue.

When the compounds (e.g., ASO molecules) of the present disclosure are administered as pharmaceuticals, to humans and animals, they can be given per se or as a pharmaceutical composition containing, for example, 0.1 to 99% (more preferably, 10 to 30%) of active ingredient in combination with a pharmaceutically acceptable carrier.

### Methods of Treatment and Administration

The ASO molecules of the present disclosure may be used to treat or prevent a disease in a subject in need thereof. In one aspect, the present disclosure provides methods of treating or preventing a disease in a subject in need thereof comprises administering to the subject any of the ASO molecules disclosed herein. In one aspect, the present disclosure provides methods of treating or preventing a disease in a subject in need thereof comprises administering to the subject any of the compositions disclosed herein.

In some embodiments of the disclosed methods and uses, the disease is a respiratory disease. In some embodiments, the respiratory disease is a viral infection. In some embodiments, the respiratory disease is viral pneumonia. In some embodiments, the respiratory disease is an acute respiratory infection. In some embodiments, the respiratory disease is a cold. In some embodiments, the respiratory disease is severe acute respiratory syndrome (SARS). In some embodiments, the respiratory disease is Middle East respiratory syndrome (MERS). In some embodiments, the disease is coronavirus disease 2019 (COVID-19). In some embodiments, the respiratory disease can include one or more symptoms selected from coughing, sore throat, runny nose, sneezing, headache, fever, shortness of breath, myalgia, abdominal pain, fatigue, difficulty breathing, persistent chest pain or pressure, difficulty waking, loss of smell and taste, muscle or joint pain, chills, nausea or vomiting, nasal congestion, diarrhea, haemoptysis, conjunctival congestion, sputum production, chest tightness, and palpitations. In some embodiments, the respiratory disease can include complications selected from sinusitis, otitis media, pneumonia, acute respiratory distress syndrome, disseminated intravascular coagulation, pericarditis, and kidney failure. In some embodiments, the respiratory disease is idiopathic.

In some embodiments, the subject is a mammal. In some embodiments, the subject is a human. In some embodiments, the subject is a non-human primate. In some embodiments, the subject is a cat. In some embodiments, the subject is a camel. In preferred embodiments in which the subject is a human, the subject may be at least 40 years old, at least 45 years old, at least 50 years old, at least 55 years old, at least 60 years old, at least 65 years old, at least 70 years old, at least 75 years old, or at least 80 years old or older. In some embodiments, the subject is a pediatric subject (i.e., less than 18 years old).

The preparations (e.g., ASO molecules or pharmaceutical compositions thereof) of the present disclosure may be given parenterally, topically, or rectally or administered in the form of an inhalant. They are, of course, given in forms suitable for each administration route. For example, they are administered in tablets or capsule form, administration by injection, infusion, or inhalation; topical by lotion or ointment; rectal by suppositories. Injection, infusion, or inhalation are preferred.

These compounds may be administered to humans and other animals for therapy or as a prophylactic by any suitable route of administration, including nasally (as by, for example, a spray), rectally, intravaginally, parenterally, intracisternally and topically, as by powders, ointments or drops, including buccally and sublingually. In some embodiments, the compounds or compositions are inhaled, as by, for example, an inhaler, a nebulizer, or in an aerosolized form.

Regardless of the route of administration selected, the compounds (e.g., ASO molecules) of the present disclosure, which may be used in a suitable hydrated form, and/or the pharmaceutical compositions of the present disclosure, are formulated into pharmaceutically-acceptable dosage forms by conventional methods known to those of skill in the art.

In some embodiments, the present disclosure provides methods of treating or preventing a coronavirus infection, comprising administering to a subject in need thereof a therapeutically effective amount of one or more of the ASOs or a pharmaceutical composition as disclosed herein. In some embodiments, the coronavirus infection is selected from the group consisting of Middle East Respiratory Syndrome (MERS), Severe Acute Respiratory Syndrome (SARS), and COVID-19. In some embodiments, the subject has been treated with one or more additional coronavirus treatment agents. In some embodiments, the subject is concurrently treated with one or more additional coronavirus treatment agents.

Actual dosage levels of the active ingredients (e.g., ASO molecules) in the pharmaceutical compositions of this disclosure may be varied so as to obtain an amount of the active ingredient which is effective to achieve the desired therapeutic response for a particular patient, composition, and mode of administration, without being toxic to the patient.

The selected dosage level will depend upon a variety of factors including the activity of the particular compound (e.g., ASO molecule) of the present disclosure employed, or the ester, salt or amide thereof, the route of administration, the time of administration, the rate of excretion or metabolism of the particular compound being employed, the rate and extent of absorption, the duration of the treatment, other drugs, compounds and/or materials used in combination with the particular compound employed, the age, sex, weight, condition, general health and prior medical history of the patient being treated, and like factors well known in the medical arts.

A physician or veterinarian having ordinary skill in the art can readily determine and prescribe the effective amount of the pharmaceutical composition required. For example, the physician or veterinarian could start doses of the compounds (e.g., ASO molecules) of the disclosure employed in the pharmaceutical composition at levels lower than that required in order to achieve the desired therapeutic effect and gradually increase the dosage until the desired effect is achieved.

In general, a suitable daily dose of a compound (e.g., ASO molecule) of the disclosure is the amount of the compound that is the lowest dose effective to produce a therapeutic effect. Such an effective dose generally depends upon the factors described above. Preferably, the compounds are administered at about 0.01 mg/kg to about 200 mg/kg, more preferably at about 0.1 mg/kg to about 100 mg/kg, even more preferably at about 0.5 mg/kg to about 50 mg/kg. In some embodiments, the compound is administered at a dose equal to or greater than 0.01, 0.02, 0.03, 0.04, 0.05, 0.06, 0.07, 0.08, 0.09, 0.10, 0.11, 0.12, 0.13, 0.14, 0.15, 0.16, 0.17, 0.18, 0.19, 0.20, 0.21, 0.22, 0.23, 0.24, 0.25, 0.26, 0.27, 0.28, 0.29, 0.30, 0.35, 0.40, 0.45, 0.50, 0.55, 0.60, 0.65, 0.7, 0.75, 0.8, 0.85, 0.9, 0.95, or 1 mg/kg. In some embodiments, the compound is administered at a dose equal to or less than 200, 190, 180, 170, 160, 150, 140, 130, 120, 110, 100, 95, 90, 85, 80, 75, 70, 65, 60, 55, 50, 45, 40, 35, 30, 25, 20, or 15 mg/kg. In some embodiments, the total daily dose of the compound is equal to or greater than 10, 15, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, or 100 mg.

The subject of the described methods may be a mammal, and it includes humans and non-human mammals. In some embodiments, the subject is a human, such as an adult human.

The disclosed ASO can be administered alone or in combination with one or more additional coronavirus treatment agents and/or antiviral agents. The additional coronavirus treatment agent and/or antiviral may be a small molecule (e.g., a nucleoside analog or a protease inhibitor) or a biologic (e.g., an antibody or peptide). Examples of suitable coronavirus treatment agents include, but are not limited to, remdesivir, favipiravir, molnupiravir, dexamethasone, bamlanivimab, casirivimab, imdevimab, convalescent plasma, and interferons. Examples of suitable antiviral agents include, but are not limited to, baloxavir marboxil, oseltamivir, anamivir, vidarabine, acyclovir, ganciclovir, zidovudine, didanosine, zalcitabine, lamivudine, saquinavir, ritonavir, indinavir, nelfinavir, ribavirin, amantadine, rimantadine, remdesivir, favipiravir, and molnupiravir.

When the compounds (e.g., ASO molecules) described herein are co-administered with another active agent, the effective amount may be less than when the compound is used alone.

If desired, the effective daily dose of the active compound (e.g., ASO molecule) may be administered as two, three, four, five, six or more sub-doses administered separately at appropriate intervals throughout the day, optionally, in unit dosage forms. Preferred dosing is one administration per day. In some embodiments, the compound is administered at least 1, 2, 3, 4, 5, 6, 7, 8, 9, 10, 11, 12, 13, 14, 15, 16, 17, 18, 19, 20, or 21 times a week. In some embodiments, the compound is administered at least 1, 2, 3, 4, 5, 6, 7, 8, 9, 10, 11, 12, 13, 14, 15, 16, 17, 18, 19, 20, or 21 times a month. In some embodiments, the compound is administered once every 1, 2, 3, 4, 5, 6, 7, 8, 9, 10, 11, 12, 13, 14, 15, 16, 17, 18, 19, 20, or 21 days. In some embodiments, the compound is administered once every 1, 2, 3, 4, 5, 6, 7, or 8 weeks.

### Examples

The following examples illustrate certain embodiments of the present disclosure to aid the skilled person in practicing the disclosure. Accordingly, the examples are in no way considered to limit the scope of the disclosure.

### Example 1: ASO Synthesis

Gapmer ASO Sequences

The DNA, 2′-O-Me, 2′-O-methoxyethylribose (2′-MOE) and LNA phosphoramidite monomers were procured from commercially available sources (Thermo Fischer Scientific or Hongene Biotech USA Inc.). All the monomers were dried in vacuum desiccator with desiccants (P₂O₅, RT 24h). Universal solid supports (CPG) attached were obtained from ChemGenes corporation. The chemicals and solvents for synthesis workflow were purchased from VWR/Sigma commercially available sources and used without any purification or treatment. Solvent (acetonitrile) and solutions (amidite and activator) were stored over molecular sieves during synthesis.

The control and target oligonucleotide sequences were synthesized on an Expedite 8909 synthesizer using the standard cycle written by the manufacturer with modifications to a few wait steps and modified coupling steps. The solid support was controlled pore glass and the monomers contained standard protecting groups. Each chimeric oligonucleotide was individually synthesized using commercially available 5′-O-(4,4′-dimethoxytrityl)-3′-O-(2-cyanoethyl-N, N-diisopropyl) DNA, 2′-OMe, 2′-MOE and/or LNA phosphoramidite monomers of 6-N-benzoyladenosine (A^{Bz}), 4-N-acetylcytidine (C^{Ac}), 4-N-5-methyl acetylcytidine (5m C^{Ac}), 2-N-isobutyrylguanosine (G^{iBu}), and Uridine (U) or Thymidine (T), according to standard solid phase Phosphoramidite synthesis protocols. The 2′-O-Me-2,6-diaminopurine phosphoramidite was purchased from Glen Research. The phosphoramidites were prepared as 0.1 M solutions in anhydrous acetonitrile. 5-Ethylthiotetrazole was used as activator, 3% dichloroacetic acid in dichloromethane was used to detritylate, acetic anhydride in THF and 16% N-methylimidazole in THF were used to cap, and DDTT ((dimethylamino-methylidene)amino)-3H-1,2,4-dithiazaoline-3-thione was used as the sulfur-transfer agent for the synthesis of oligoribonucleotide phosphorothioates. An extended coupling of 0.1M solution of phosphoramidite in CH₃CN in the presence of 5-(ethylthio)-1H-tetrazole activator to a solid bound oligonucleotide followed by extended capping, oxidation and deprotection afforded modified oligonucleotides. The stepwise coupling efficiency of all modified phosphoramidites was more than 98.5%.

Deprotection and cleavage from the solid support was achieved with mixture of ammonia methylamine (1:1, AMA) for 15 min at 65° C., when the universal linker was used, the deprotection was left for 90 min at 65° C. or solid supports were heated with aqueous ammonia (28%) solution at 55° C. for 8 h to deprotect the base labile protecting groups.

After filtering to remove the solid support, the deprotection solution was removed under vacuum in a GeneVac centrifugal evaporator.

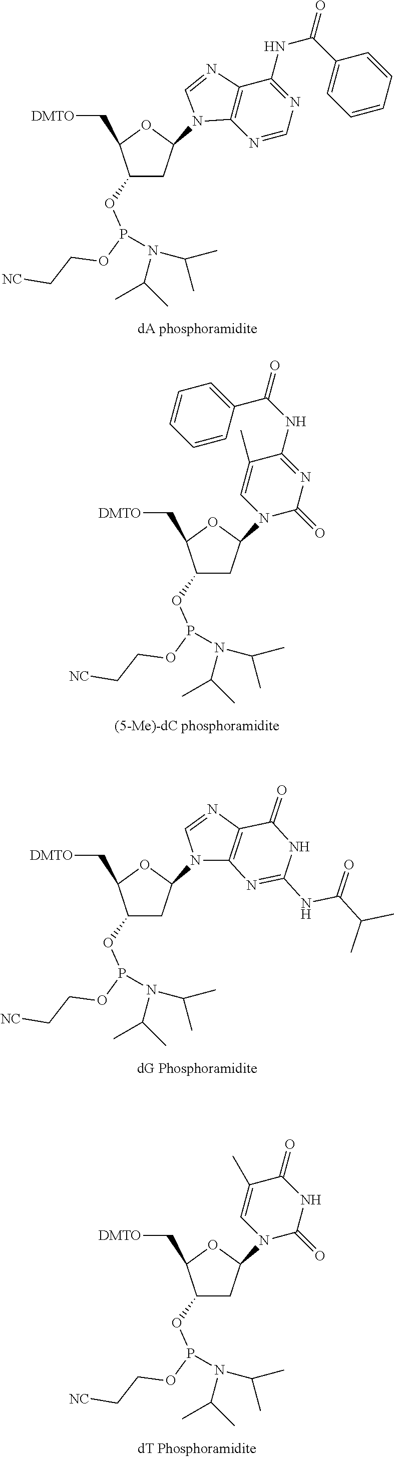

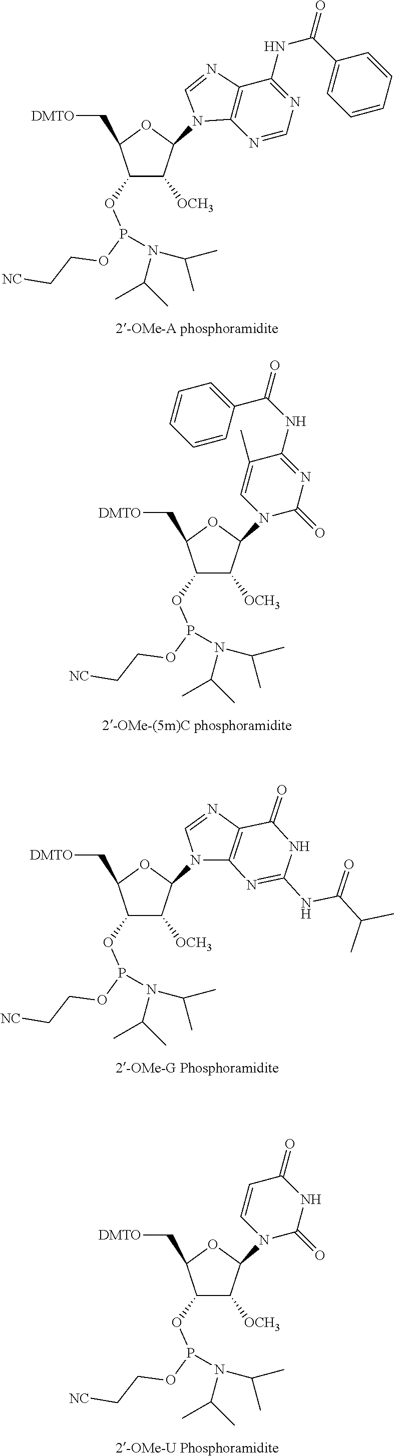

The 2′-MOE phosphoramidites

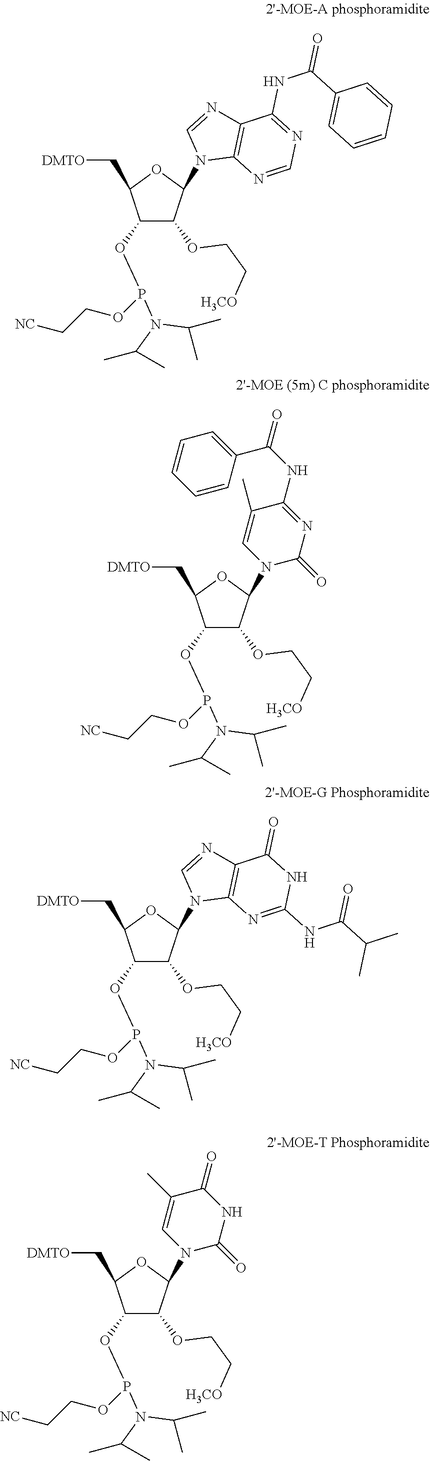

The Locked nucleic acid (LNA) phosphoramidite

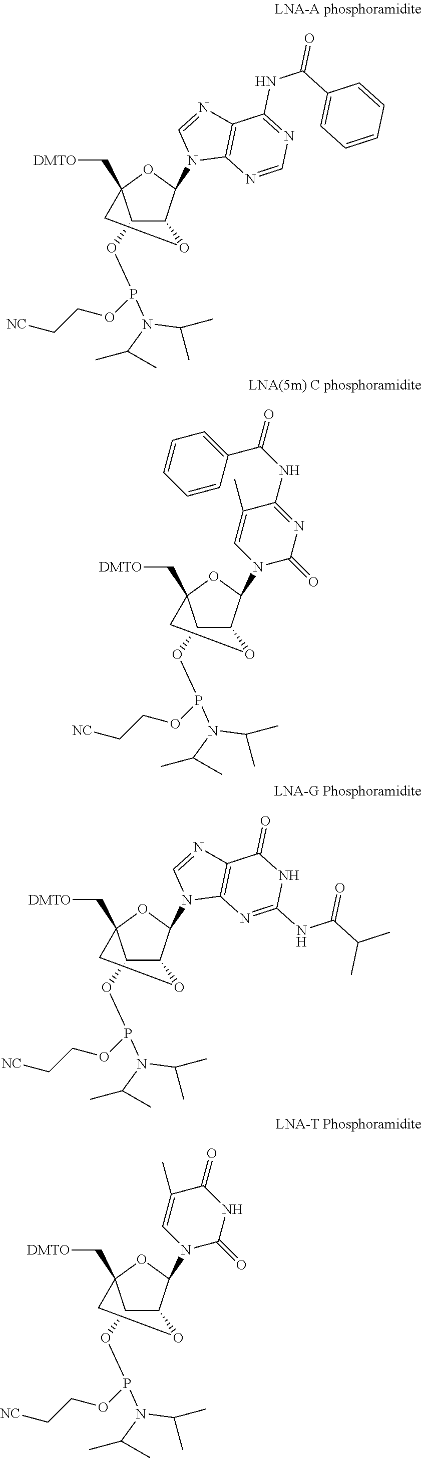

Modified Gapmer Sequences

The AmNA (N-Me)-T, AmNA (N-Me)-4-N-benzoyl (5m) cytidine ((5m) C^{Bz}), AmNA (N-Me)-4-N-benzoylcytidine (A^{Bz}), and AmNA (N-Me)-2-N-pac (G^{pac}), were purchased from Luxna Biotech, whereas scp-BNA-T, scp-BNA-6-N-benzoyladenosine (A^{Bz}), scp-BNA-4-N-benzoyl-5 methyl cytidine ((5m) C^{Bz}), scp-BNA-2-N-iguanosine (G^{iBu}) phosphoramidite monomers synthesized by following the procedure described in references (Takao Yamaguchi, Masahiko Horiba and Satoshi Obika; Chem. Commun., 2015, 51, 9737-9740; Masahiko Horiba, Takao Yamaguchi, and Satoshi Obika; Journal of Organic Chemistry, 2016, 81, 11000-11008). All the monomers were dried in a vacuum desiccator with desiccants (KOH and P₂O₅, at room temperature for 24 hours). In the case of AmNA(N-Me)-PS-DNA-PS and scp-BNA-PS-DNA-PS, modifications the synthesis was carried out on a 1 μM scale in a 3′ to 5′ direction with the phosphoramidite monomers diluted to a concentration of 0.12 M in anhydrous CH₃CN in the presence of 0.3 M 5-(benzylthio)-1H-tetrazole activator (coupling time 16 min) to a solid bound oligonucleotide followed by modified capping, oxidation and deprotection afforded modified oligonucleotides. The stepwise coupling efficiency of all modified phosphoramidites was more than 97%. The DDTT (dimethylamino-methylidene) amino)-3H-1,2,4-dithiazaoline-3-thione was used as the sulfur-transfer agent for the synthesis of oligoribonucleotide phosphorothioates. Oligonucleotide-bearing solid supports were washed with 20% DEA solution in acetonitrile for 15 min then column was washed thoroughly with MeCN. The support was heated at 65° C. with diisopropylamine:water:methanol (1:1:2) for 8 h in heat block to cleavage from support and deprotect the base labile protecting groups.

### AmNA (N-Me) Monomers

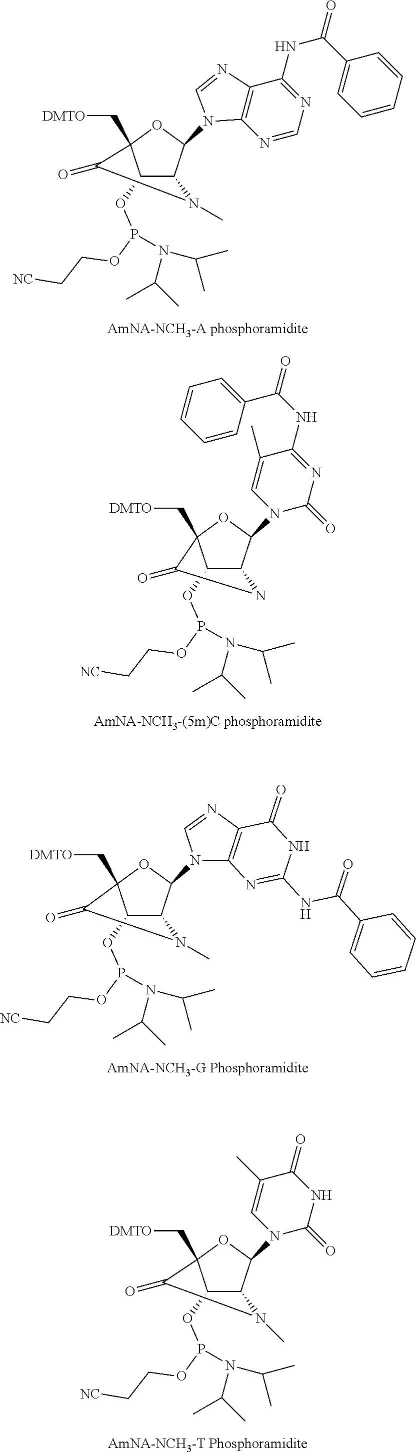

scp-BNA monomers

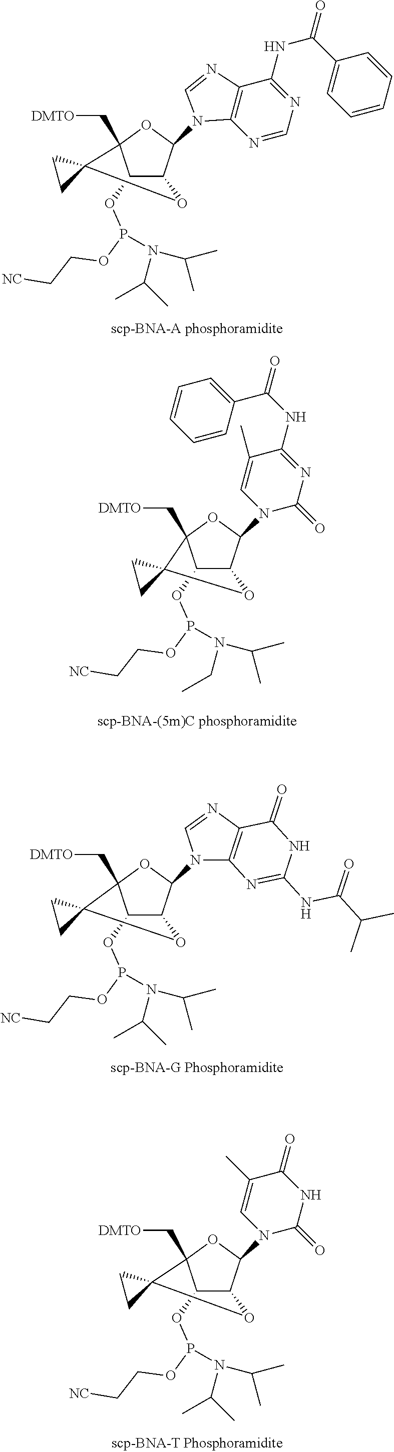

Quantitation of Crude Oligomer or Raw Analysis

Samples were dissolved in deionized water (1.0 mL) and quantitated as follows: Blanking was first performed with water alone on Nanodrop UV spectrophotometer. Nano Drop instruments can measure a wide concentration range of nucleic acids through use of multiple path lengths. The most accurate quantification results can be achieved by measuring diluted oligonucleotides with an absorbance at 260 nm. The crude material is stored at −20° C.

Crude HPLC/LC-MS Analysis

The 0.1 OD of the crude samples were used for crude MS analysis. After confirming the crude LC-MS data, then the purification step was performed.

HPLC Purification

The Phosphodiester (PO), Phosphorothioate (PS) and chimeric modified oligonucleotides were purified by anion-exchange HPLC. The buffers were 20 mM sodium phosphate in 10% CH₃CN, pH 8.5 (buffer A) and 20 mM sodium phosphate in 10% CH₃CN, 1.8 M NaBr, pH 8.5 (buffer B). Fractions containing full-length oligonucleotides were pooled, desalted and lyophilized.

The lipid conjugated oligonucleotides were purified by an in-house packed RPC-Source15 reverse-phase column. The buffers were 20 mM sodium acetate in 10% CH₃CN, (buffer A) and CH₃CN (buffer B). Fractions containing full-length oligonucleotides were pooled, desalted and lyophilized.

Desalting of Purified Oligomer

The purified dry oligomer was then desalted using Sephadex G-25 M (Amersham Biosciences). The cartridge was conditioned with 10 mL of deionized water thrice. The purified oligonucleotide dissolved thoroughly in 2.5 mL deionized water was applied to the cartridge with very slow drop wise elution. The salt free oligomer was eluted with 3.5 ml deionized water directly into a screw cap vial.

Final HPLC and Electrospray LC/MS Analysis

Approximately 0.10 OD of oligomer is dissolved in water and then pipetted in special vials for IEX-HPLC and LC/MS analysis. Analytical HPLC and ES LC-MS established the integrity of the chimeric oligonucleotides.

### Example 2: Human Beta-Coronavirus OC43 Transfection-Based Antiviral Assay in HeLa Cells

The human beta-coronavirus OC43 was purchased from ATCC (Manassas, Va.) and propagated using HCT-8 human colorectal epithelial cells (ATCC). HeLa human cervical epithelial cells (ATCC) was used as susceptible host cell lines and were cultured using EMEM media, supplemented with 10% fetal bovine serum (FBS), 1% (v/v) penicillin/streptomycin (P/S), 1% (v/v) HEPES and 1% (v/v) cellgro Glutagro™ supplement (all Corning, Manassas, Va.) at 37° C. For the OC43 antiviral assay, 1.5×10⁴ HeLa cells per well were plated in 100 μl complete media in white 96-well plates with clear flat bottom at 37° C. for up to 24 hours to facilitate attachment and allow cells to recover from seeding stresses. Next day, HeLa cells were inoculated with 100 ul of HCoV-OC43 stock diluted to a concentration known to produce optimal cytopathic effect, inducing 80-90% reduction in cell viability. Cells were incubated at 33° C. in a humidified atmosphere 5% CO₂ for about 2.5-3 hrs in 2% FBS assay media (EMEM, 2% FBS, 1% P/S, 1% cellgro Glutagro™ supplement, 1% HEPES). Cells were then washed twice with phosphate-buffered saline (PBS) and EMEM supplemented with 2% FBS was added to cells. At the same time, cells were also transfected with test antisense oligonucleotides (ASO) in duplicates using 0.3 ul/well RNAiMAX transfection reagent (1:1 ratio; Invitrogen) according to the manufacturer's protocol and incubated for 48 hrs at 33° C. in a humidified atmosphere 5% CO₂; each plate contains uninfected control wells as well as virus-infected mock transfection wells that were not treated with oligonucleotides. The final concentration of each ASO was 1 μM. Supernatant was collected 48 hrs post incubation for extracellular viral RNA quantification via quantigene bDNA assay (Invitrogen probe set and reagents) according to manufacturer's protocol. Data was collected on Luminometer (Envision plate reader) and reported as antiviral % inhibition at 1 μM was calculated as follows: 100×(oligonucleotide/infected−uninfected)/(infected/untreated−uninfected). Cytotoxicity plates without the addition of OC43 virus were carried out with the same assay format on a different day. After 48 hrs incubation, cells were assayed and lysed with 100 μl cell-titer-glo reagent (Promega, Madison, Wis.) and incubated for at least 10 min at room temperature prior to measuring luminescence. Luminescence was measured on a Perkin Elmer (Waltham, Mass.) Envision plate reader.

The results of these assays are shown in Table 4 below. The data was reported as viability relative to no-drug control (mock transfection using 0.3 ul/well RNAiMAX transfection reagent).

TABLE 4
Biological Activity of Exemplary ASOs
in Antiviral Assay in HeLa Cells

Human beta-coronavirus OC43 transfection-
based antiviral assay in HeLa cells

| | % Virus | % Cell |
|---|---|---|
| | inhibition | Viability |
| | at 1 uM; | at 1 Um; |
| | A: >80%; | A: >80%; |
| | B: 50-79%, | B: 50-79%, |
| Name | C: <50% | C: <50% |
| ASO-000 | C | B |
| ASO-001 | n.d. | A |
| ASO-002 | C | B |
| ASO-003 | A | C |
| ASO-004 | B | C |
| ASO-005 | A | C |
| ASO-006 | B | C |
| ASO-007 | C | C |
| ASO-008 | C | C |
| ASO-009 | B | B |
| ASO-010 | B | C |
| ASO-011 | A | C |
| ASO-012 | B | C |
| ASO-013 | C | B |
| ASO-014 | A | C |
| ASO-015 | C | A |
| ASO-016 | B | C |
| ASO-017 | B | C |
| ASO-018 | C | C |
| ASO-019 | C | C |
| ASO-020 | C | C |
| ASO-021 | A | C |
| ASO-022 | C | A |
| ASO-023 | C | B |
| ASO-024 | B | C |
| ASO-025 | A | C |
| ASO-026 | A | C |
| ASO-027 | A | C |
| ASO-028 | A | C |
| ASO-029 | B | C |
| ASO-030 | A | C |
| ASO-031 | A | C |
| ASO-032 | C | C |
| ASO-033 | B | C |
| ASO-034 | B | C |
| ASO-035 | C | B |
| ASO-036 | C | B |
| ASO-037 | n.d. | n.d. |
| ASO-038 | B | B |
| ASO-039 | C | A |
| ASO-040 | A | C |
| ASO-041 | A | C |
| ASO-042 | A | C |
| ASO-043 | A | C |
| ASO-044 | A | C |
| ASO-045 | C | A |
| ASO-046 | B | B |
| ASO-047 | B | B |
| ASO-048 | A | C |
| ASO-049 | B | C |
| ASO-050 | A | C |
| ASO-051 | A | C |
| ASO-052 | A | C |
| ASO-053 | A | C |
| ASO-054 | A | B |
| ASO-055 | A | C |
| ASO-056 | A | C |
| ASO-057 | A | C |
| ASO-058 | C | A |
| ASO-059 | B | A |
| ASO-060 | A | C |
| ASO-061 | A | C |
| ASO-062 | A | C |
| ASO-063 | A | C |
| ASO-064 | A | C |
| ASO-065 | A | C |
| ASO-066 | A | C |
| ASO-067 | A | C |
| ASO-068 | n.d. | n.d. |
| ASO-069 | A | C |
| ASO-070 | A | C |
| ASO-071 | C | B |
| ASO-072 | A | C |
| ASO-073 | A | C |
| ASO-074 | n.d. | n.d. |
| ASO-075 | C | B |
| ASO-076 | B | C |
| ASO-077 | A | C |
| ASO-078 | C | B |
| ASO-079 | A | C |
| ASO-080 | A | C |
| ASO-081 | A | C |
| ASO-082 | A | C |
| ASO-083 | B | C |
| ASO-084 | A | C |
| ASO-085 | A | C |
| ASO-086 | A | C |
| ASO-087 | A | C |
| ASO-088 | A | C |
| ASO-089 | A | C |
| ASO-090 | C | A |
| ASO-091 | B | C |
| ASO-092 | C | A |
| ASO-093 | A | C |
| ASO-094 | C | A |
| ASO-095 | A | C |
| ASO-096 | A | C |
| ASO-097 | C | A |
| ASO-098 | A | B |
| ASO-099 | A | C |
| ASO-100 | B | B |
| ASO-101 | A | C |
| ASO-102 | A | C |
| ASO-103 | A | C |
| ASO-104 | A | C |
| ASO-105 | A | C |
| ASO-106 | C | A |
| ASO-107 | C | B |
| ASO-108 | A | C |
| ASO-109 | A | C |
| ASO-110 | A | C |
| ASO-111 | A | C |
| ASO-112 | A | C |
| ASO-113 | A | C |
| ASO-114 | A | C |
| ASO-115 | A | C |
| ASO-116 | A | C |
| ASO-117 | n.d. | n.d. |
| ASO-118 | n.d. | n.d. |
| ASO-119 | n.d. | n.d. |
| ASO-120 | A | B |
| ASO-121 | A | B |
| ASO-122 | A | C |
| ASO-123 | A | C |
| ASO-124 | A | C |
| ASO-125 | A | C |
| ASO-126 | A | C |
| ASO-127 | A | C |
| ASO-128 | n.d. | n.d. |
| ASO-129 | C | A |
| ASO-130 | A | C |
| ASO-131 | A | C |
| ASO-132 | A | B |
| ASO-133 | C | A |
| ASO-134 | A | C |
| ASO-135 | A | C |
| ASO-136 | A | C |
| ASO-137 | A | C |
| ASO-138 | B | B |
| ASO-139 | A | C |
| ASO-140 | A | C |
| ASO-141 | A | C |
| ASO-142 | B | B |

### Example 3: Reporter Plasmid Luciferase and Cytotoxicity Assay in Cos7 Cells

COS-7 monkey fibroblast cells (ATCC, CRL-1651) were seeded into 96-well culture plates at 15.0×10⁴ cells/well and cultured in Dulbecco's Modified Eagle's Medium (DMEM; Hyclone, SH30022) supplemented with 10% fetal bovine serum (FBS; Sigma-Aldrich, F4135) and 1% Penicillin-Streptomycin (P/S; Corning, 30-002-CI) at 37° C. and 5% CO₂. After 6 hrs of incubation, cells were transiently transfected with psiCHECK2-SARS-CoV-2 plasmid (custom-synthesized by Genscript) at 50 ng/well using 0.3 μL of Lipofectamine 3000 transfection reagent (1:1 reagent/psi-CHECK2-SARS-CoV-2 DNA ratio; Invitrogen) in Opti-MEM (Invitrogen, 11058-021) according to the manufacturer's protocol. After overnight incubation, the medium was removed and replaced with 100 ul fresh growth media. Test oligonucleotides along with appropriate controls (Ambion siRNAs, ThermoFisher) were serially diluted in Opti-MEM (Invitrogen, 11058-021). Cells were then transfected with test siRNAs in duplicates using 0.3 ul/well RNAiMAX transfection reagent (1:1 ratio; Invitrogen) according to the manufacturer's protocol. After approximately 48 hrs, the culture plates were equilibrated to RT, 100 μL of Dual-Luciferase Reporter Assay reagent (Promega, E6120) were added to each well according to manufacturer's protocol. Luminescence was measured on an Envision plate reader (Perkin Elmer). The results were then quantified by calculating the ratio of renilla to firefly luciferase expression for each of the duplicates and reported as percent inhibition of luciferase activity relative to no-drug control (mock transfection with psiCHECK2-SARS-CoV-2 plasmid) and dose-response curves were fitted by non-linear regression with variable slope (four parameters). Statistical analysis was performed in GraphPad Prism 8.3.1 (San Diego, Calif.) and the EC₅₀ was calculated which is the predicted oligonucleotide concentration corresponding to a 50% inhibition of the luciferase activity. The assay was repeated with a different set of plates and cytotoxicity of test siRNAs was assessed 48 hrs post treatment of COS-7 cells. The cells were lysed and assayed with Cell-Titer Glo reagent (Promega) according to the manufacturer's protocol.

The results of these assays are shown in Table 5 below in the EC₅₀ column. The data was reported as % viability relative to no-drug control (mock transfection with psiCHECK2-SARS-CoV-2 plasmid) and dose-response curves were fitted by non-linear regression with variable slope (four parameters) using GraphPad prism software version 8.3.1.

### Example 4: Cytotoxicity Assessment in A549 Cells

A549 human lung epithelial cells (ATCC, CCL-185) were seeded into 96-well F-bottom white-walled culture plates at 1.20×10⁴ cells/well and cultured in 100 ul of Ham's F12 1× (Modified) WITH L-glutamine (Corning; 10-080-CM) supplemented with 10% fetal bovine serum (FBS; Sigma-Aldrich, F4135) and 1% Penicillin-Streptomycin (P/S; Corning, 30-002-CI) at 37° C. and 5% CO₂ for up to 24 hrs to facilitate attachment. After overnight incubation, the medium was removed and replaced with 100 ul fresh growth media with 2% FBS. Test siRNAs or ASOs along with appropriate controls were serially diluted in Opti-MEM (Invitrogen, 11058-021). Cells were then transfected with test siRNAs or ASOs in duplicates using 0.2 ul/well RNAiMAX transfection reagent (1:1 ratio; Invitrogen) according to the manufacturer's protocol. After approximately 48 hrs, the culture plates were equilibrated to RT. Cells treated with siRNAs were lysed and assayed with 100 ul/well Cell-Titer Glo reagent (Promega) according to the manufacturer's protocol. After incubation for at least 10 min at room temperature, luminescence was measure using Envision plate reader (Perkin Elmer). The data was reported as % viability relative to no-drug control (mock transfection with psiCHECK2-SARS-CoV-2 plasmid) and dose-response curves were fitted by non-linear regression with variable slope (four parameters) using GraphPad prism software version 8.3.1. On the other hand, cells treated with ASOs were lysed and assayed for detection of caspase-3 and -7 activities. 1:1 ratio of Caspase-Glo 3/7 reagent volume (Promega) was added to sample volume, plate contents were gently mixed using a plate-shaker at 300-500 rpm for 30 seconds and were incubated for approximately 1 hr at room temperature before recording luminescence.

The results of these assays are shown in Table 5 below in the CC₅₀ column. Data was collected on Luminometer (Envision plate reader) and reported as % apoptosis relative to no-drug control (mock transfection with 0.2 ul of RNAiMAX transfection reagent), subtracting background signal from every well. Increased caspase activation with cytotoxicity indicated cytotoxicity is the result of apoptosis; dose-response curves were fitted by non-linear regression with variable slope (four parameters) using GraphPad prism software version 8.3.1.

TABLE 5
EC₅₀ and CC₅₀ Data for Exemplary ASOs

| | EC₅₀ in psiCHECK2 | CC₅₀ (apoptosis) |
|---|---|---|
| | reporter assay; | in A549 cells; |
| | A: <5 nM; | A: >100 nM CC₅₀; |
| | B: 5.1-10 nM; | B: 50-100 nM CC₅₀; |
| Name | C: >10 nM | C: <50 nM CC₅₀ |
| ASO-143 | B | A |
| ASO-144 | B | A |
| ASO-145 | B | B |
| ASO-146 | A | B |
| ASO-147 | B | A |
| ASO-148 | C | C |
| ASO-149 | A | C |
| ASO-150 | B | C |
| ASO-151 | B | C |
| ASO-152 | B | C |
| ASO-153 | C | A |
| ASO-154 | B | B |
| ASO-155 | A | B |
| ASO-156 | C | B |
| ASO-157 | C | A |
| ASO-158 | A | n.d. |
| ASO-159 | C | n.d. |
| ASO-160 | C | n.d. |
| ASO-161 | C | n.d. |
| ASO-162 | C | n.d. |
| ASO-163 | A | A |
| ASO-164 | B | A |
| ASO-165 | C | A |
| ASO-166 | B | A |
| ASO-167 | B | C |
| ASO-168 | C | C |
| ASO-169 | C | A |
| ASO-170 | C | C |
| ASO-171 | C | C |
| ASO-172 | A | C |
| ASO-173 | B | A |
| ASO-174 | C | B |
| ASO-175 | B | B |
| ASO-176 | C | A |
| ASO-177 | A | A |
| ASO-178 | A | C |
| ASO-179 | C | A |

All patents and publications mentioned in the specification are indicative of the levels of those of ordinary skill in the art to which the disclosure pertains. All patents and publications are herein incorporated by reference to the same extent as if each individual publication was specifically and individually indicated to be incorporated by reference.

Further, one skilled in the art readily appreciates that the present disclosure is well adapted to carry out the objects and obtain the ends and advantages mentioned, as well as those inherent therein. Modifications therein and other uses will occur to those skilled in the art. These modifications are encompassed within the spirit of the disclosure and are defined by the scope of the claims, which set forth non-limiting embodiments of the disclosure.

TABLE 2
Exemplary Coronavirus Genomes

| SEQ ID NO: | Description | Sequence |
|---|---|---|
| 5535 | SARS-CoV-2 genome | attaaaggtttataccttcccaggtaacaaaccaaccaactttcgatctcttgtagatctgttctctaaacgaactttaaaatctgtgtggctgtcact |
| | Genbank Accession No. NC_045512.2 | cggctgcatgcttagtgcactcacgcagtataattaataactaattactgtcgttgacaggacacgagtaactcgtctatcttctgcaggctgcttacg |
| | | gtttcgtccgtgttgcagccgatcatcagcacatctaggtttcgtccgggtgtgaccgaaaggtaagatggagagccttgtccctggtttcaacgagaa |
| | | aacacacgtccaactcagtttgcctgttttacaggttcgcgacgtgctcgtacgtggctttggagactccgtggaggaggtcttatcagaggcacgtca |
| | | acatcttaaagatggcacttgtggcttagtagaagttgaaaaaggcgttttgcctcaacttgaacagccctatgtgttcatcaaacgttcggatgctcg |
| | | aactgcacctcatggtcatgttatggttgagctggtagcagaactcgaaggcattcagtacggtcgtagtggtgagacacttggtgtccttgtccctca |
| | | tgtgggcgaaataccagtggcttaccgcaaggttcttcttcgtaagaacggtaataaaggagctggtggccatagttacggcgccgatctaaagtcatt |
| | | tgacttaggcgacgagcttggcactgatccttatgaagattttcaagaaaactggaacactaaacatagcagtggtgttacccgtgaactcatgcgtga |
| | | gcttaacggaggggcatacactcgctatgtcgataacaacttctgtggccctgatggctaccctcttgagtgcattaaagaccttctagcacgtgctgg |
| | | taaagcttcatgcactttgtccgaacaactggactttattgacactaagaggggtgtatactgctgccgtgaacatgagcatgaaattgcttggtacac |
| | | ggaacgttctgaaaagagctatgaattgcagacaccttttgaaattaaattggcaaagaaatttgacaccttcaatggggaatgtccaaattttgtatt |
| | | tcccttaaattccataatcaagactattcaaccaagggttgaaaagaaaaagcttgatggctttatgggtagaattcgatctgtctatccagttgcgtc |
| | | accaaatgaatgcaaccaaatgtgcctttcaactctcatgaagtgtgatcattgtggtgaaacttcatggcagacgggcgattttgttaaagccacttg |
| | | cgaattttgtggcactgagaatttgactaaagaaggtgccactacttgtggttacttaccccaaaatgctgttgttaaaatttattgtccagcatgtca |
| | | caattcagaagtaggacctgagcatagtcttgccgaataccataatgaatctggcttgaaaaccattcttcgtaagggtggtcgcactattgcattgga |
| | | ggctgtgtgttctcttatgttggttgccataacaagtgtgcctattgggttccacgtgctagcgctaacataggttgtaaccatacaggtgttgttgga |
| | | gaaggttccgaaggtcttaatgacaaccttcttgaaatactccaaaaagagaaagtcaacatcaatattgttggtgactttaaacttaatgaagagatc |
| | | gccattattttggcatctttttctgcttccacaagtgcttttgtggaaactgtgaaaggtttggattataaagcattcaaacaaattgttgaatcctgt |
| | | ggtaattttaaagttacaaaaggaaaagctaaaaaaggtgcctggaatattggtgaacagaaatcaatactgagtcctattatgcatttgcatcagagg |
| | | ctgctcgtgttgtacgatcaattttctcccgcactcttgaaactgctcaaaattctgtgcgtgttttacagaaggccgctataacaatactagatggaa |
| | | tttcacagtattcactgagactcattgatgctatgatgttcacatctgatttggctactaacaatctagttgtaatggcctacattacaggtggtgttg |
| | | ttcagttgacttcgcagtggctaactaacatctttggcactgtttatgaaaaactcaaacccgtccttgattggcttgaagagaagtttaaggaaggtg |
| | | tagagtttcttagagacggttgggaaattgttaaatttatctcaacctgtgcttgtgaaattgtcggtggacaaattgtcacctgtgcaaaggaaatta |
| | | aggagagtgttcagacattattaagcttgtaaataaatttttggctttttgtgtgctgactctatcattattggtggagctaaacttaaagccttgaat |
| | | ttaggtgaaacatttgtcacgcactcaaagggattgtacagaaagtgtgttaaatccagagaagaaactggcctactcatgcctctaaaagccccaaaa |
| | | gaaattatcttcttagagggagaaacacttcccacagaagtgttaacagaggaagttgtcttgaaaactggtgatttacaaccattagaacaacctact |
| | | agtgaagctgttgaagctccattggttggtacaccagtttgtattaacgggcttatgttgctcgaaatcaaagacacagaaaagtactgtgcccttgca |
| | | cctaatatgatggtaacaaacaataccttcacactcaaaggcggtgcaccaacaaaggttacttttggtgatgacactgtgatagaagtgcaaggttac |
| | | aagagtgtgaatatcacttttgaacttgatgaaaggattgataaagtacttaatgagaagtgctctgcctatacagttgaactcggtacagaagtaaat |
| | | gagttcgcctgtgttgtggcagatgctgtcataaaaactttgcaaccagtatctgaattacttacaccactgggcattgatttagatgagtggagtatg |
| | | gctacatactacttatttgatgagtctggtgagtttaaattggcttcacatatgtattgttctttctaccctccagatgaggatgaagaagaaggtgat |
| | | tgtgaagaagaagagtttgagccatcaactcaatatgagtatggtactgaagatgattaccaaggtaaacctttggaatttggtgccacttctgctgct |
| | | cttcaacctgaagaagagcaagaagaagattggttagatgatgatagtcaacaaactgttggtcaacaagacggcagtgaggacaatcagacaactact |
| | | attcaaacaattgttgaggttcaacctcaattagagatggaacttacaccagttgttcagactattgaagtgaatagttttagtggttatttaaaactt |
| | | actgacaatgtatacattaaaaatgcagacattgtggaagaagctaaaaaggtaaaaccaacagtggttgttaatgcagccaatgtttaccttaaacat |
| | | ggaggaggtgttgcaggagccttaaataaggctactaacaatgccatgcaagttgaatctgatgattacatagctactaatggaccacttaaagtgggt |
| | | ggtagttgtgttttaagcggacacaatcttgctaaacactgtcttcatgttgtcggcccaaatgttaacaaaggtgaagacattcaacttcttaagagt |
| | | gcttatgaaaattttaatcagcacgaagttctacttgcaccattattatcagctggtatttttggtgctgaccctatacattattaagagtttgtgtag |
| | | atactgttcgcacaaatgtctacttagctgtattgataaaaatctctatgacaaacttgtttcaagattttggaaatgaagagtgaaaagcaagttgaa |
| | | caaaagatcgctgagattcctaaagaggaagttaagccatttataactgaaagtaaaccttcagttgaacagagaaaacaagatgataagaaaatcaaa |
| | | gcttgtgttgaagaagttacaacaactaggaagaaactaagttcctcacagaaaacttgttactttatattgacattaatggcaatcttcatccagatt |
| | | ctgccactcttgttagtgacattgacatcactttcttaaagaaagatgaccatatatagtgggtgatgttgttcaagagggtgttttaactgctgtggt |
| | | tatacctactaaaaaggctggtggcactactgaaatgctagcgaaagattgagaaaagtgccaacagacaattatataaccacttacccgggtcagggt |
| | | ttaaatggttacactgtagaggaggcaaagacagtgcttaaaaagtgtaaaagtgccttttacattctaccatctattatctctaatgagaagcaagaa |
| | | attcttggaactgtttcttggaatttgcgagaaatgcttgcacatgcagaagaaacacgcaaattaatgcctgtctgtgtggaaactaaagccatagtt |
| | | tcaactatacagcgtaaatataagggtattaaaatacaagagggtgtggttgattatggtgctagattttacttttacaccagtaaaacaactgtagcg |
| | | tcacttatcaacacacttaacgatctaaatgaaactcttgttacaatgccacttggctatgtaacacatggcttaaatttggaagaagctgctcggtat |
| | | atgagatctctcaaagtgccagctacagtttctgtttcttcacctgatgctgttacagcgtataatggttatcttacttcttcttctaaaacacctgaa |
| | | gaacattttattgaaaccatctcacttgctggttcctataaagattggtcctattctggacaatctacacaactaggtatagaatttcttaagagaggt |
| | | gataaaagtgtatattacactagtaatcctaccacattccacctagatggtgaagttatcacctttgacaatcttaagacacttctttattgagagaag |
| | | tgaggactattaaggtgtttacaacagtagacaacattaacctccacacgcaagttgtggacatgtcaatgacatatggacaacagtttggtccaactt |
| | | atttggatggagctgatgttactaaaataaaacctcataattcacatgaaggtaaaacattttatgttttacctaatgatgacactctacgtgttgagg |
| | | cttttgagtactaccacacaactgatcctagttttctgggtaggtacatgtcagcattaaatcacactaaaaagtggaaatacccacaagttaatggtt |
| | | taacttctattaaatgggcagataacaactgttatcttgccactgcattgttaacactccaacaaatagagttgaagtttaatccacctgctctacaag |
| | | atgcttattacagagcaagggctggtgaagctgctaacttttgtgcacttatcttagcctactgtaataagacagtaggtgagttaggtgatgttagag |
| | | aaacaatgagttacttgtttcaacatgccaatttagattcttgcaaaagagtcttgaacgtggtgtgtaaaacttgtggacaacagcagacaaccctta |
| | | agggtgtagaagctgttatgtacatgggcacactttcttatgaacaatttaagaaaggtgttcagataccttgtacgtgtggtaaacaagctacaaaat |
| | | atctagtacaacaggagtcaccttttgttatgatgtcagcaccacctgctcagtatgaacttaagcatggtacatttacttgtgctagtgagtacactg |
| | | gtaattaccagtgtggtcactataaacatataacttctaaagaaactttgtattgcatagacggtgctttacttacaaagtcctcagaatacaaaggtc |
| | | ctattacggatgttttctacaaagaaaacagttacacaacaaccataaaaccagttacttataaattggatggtgttgtttgtacagaaattgacccta |
| | | agttggacaattattataagaaagacaattcttatttcacagagcaaccaattgatcttgtaccaaaccaaccatatccaaacgcaagcttcgataatt |
| | | ttaagtttgtatgtgataatatcaaatttgctgatgatttaaaccagttaactggttataagaaacctgcttcaagagagcttaaagttacatttttcc |
| | | ctgacttaaatggtgatgtggtggctattgattataaacactacacaccacttttaagaaaggagctaaattgttacataaacctattgtttggcatgt |
| | | taacaatgcaactaataaagccacgtataaaccaaatacctggtgtatacgttgtattggagcacaaaaccagttgaaacatcaaattcgtttgatgta |
| | | ctgaagtcagaggacgcgcagggaatggataatcttgcctgcgaagatctaaaaccagtctctgaagaagtagtggaaaatcctaccatacagaaagac |
| | | gttcttgagtgtaatgtgaaaactaccgaagttgtaggagacattatacttaaaccagcaaataatagtttaaaaattacagaagaggttggccacaca |
| | | gatctaatggctgcttatgtagacaattctagtcttactattaagaaacctaatgaattatctagagtattaggtttgaaaaccdtgctactcatggtt |
| | | tagctgctgttaatagtgtccdtgggatactatagctaattatgctaagccttttcttaacaaagttgttagtacaactactaacatagttacacggtg |
| | | tttaaaccgtgtttgtactaattatatgccttatttctttactttattgctacaattgtgtacttttactagaagtacaaattctagaattaaagcatc |
| | | tatgccgactactatagcaaagaatactgttaagagtgtcggtaaattttgtctagaggcttcatttaattatttgaagtcacctaatttttctaaact |
| | | gataaatattataatttggtttttactattaagtgtttgcctaggttattaatctactcaaccgctgattaggtgttttaatgtctaatttaggcatgc |
| | | cttcttactgtactggttacagagaaggctatttgaactctactaatgtcactattgcaacctactgtactggttctataccttgtagtgtttgtctta |
| | | gtggtttagattattagacacctatccttattagaaactatacaaattaccatttcatatttaaatgggatttaactgcttttggcttagttgcagagt |
| | | ggtttttggcatatattcttttcactaggtttttctatgtacttggattggctgcaatcatgcaattgtttttcagctattttgcagtacattttatta |
| | | gtaattcttggcttatgtggttaataattaatcttgtacaaatggccccgatttcagctatggttagaatgtacatcttattgcatcattttattatgt |
| | | atggaaaagttatgtgcatgttgtagacggttgtaattcatcaacttgtatgatgtgttacaaacgtaatagagcaacaagagtcgaatgtacaactat |
| | | tgttaatggtgttagaaggtccttttatgtctatgctaatggaggtaaaggcttttgcaaactacacaattggaattgtgttaattgtgatacattctg |
| | | tgctggtagtacatttattagtgatgaagttgcgagagacttgtcactacagtttaaaagaccaataaatcctactgaccagtcttcttacatcgttga |
| | | tagtgttacagtgaagaatggttccatccatattactttgataaagaggtcaaaagacttatgaaagacattactctctcattttgttaacttagacaa |
| | | cctgagagctaataacactaaaggttcattgcctattaatgttatagtttttgatggtaaatcaaaatgtgaagaatcatctgcaaaatcagcgtagtt |
| | | tactacagtcagcttatgtgtcaacctatactgttactagatcaggcattagtgtctgatgttggtgatagtgcggaagttgcagttaaaatgtttgat |
| | | gcttacgttaatacgttttcatcaacttttaacgtaccaatggaaaaactcaaaacactagttgcaactgcagaagctgaacttgcaaagaatgtgtcc |
| | | ttagacaatgtcttatctacttttatttcagcagctcggcaagggtttgttgattcagatgtagaaactaaagatgttgttgaatgtcttaaattgtca |
| | | catcaatctgacatagaagttactggcgatagttgtaataactatatgctcacctataacaaagttgaaaacatgacaccccgtgaccttggtgcttgt |
| | | attgactgtagtgcgcgtcatattaatgcgcaggtagcaaaaagtcacaacattgattgatatggaacgttaaagatttcatgtcattgtctgaacaac |
| | | tacgaaaacaaatacgtagtgctgctaaaaagaataacttaccttttaagttgacatgtgcaactactagacaagttgttaatgttgtaacaacaaaga |
| | | tagcacttaagggtggtaaaattgttaataattggttgaagcagttaattaaagttacacttgtgttcctttttgttgctgctattttctatttaataa |
| | | cacctgttcatgtcatgtctaaacatactgacttttcaagtgaaatcataggatacaaggctattgatggtggtgtcactcgtgacatagcatctacag |
| | | atacttgttttgctaacaaacatgctgattttgacacatggtttagccagcgtggtggtagttatactaatgacaaagcttgcccattgattgctgcag |
| | | tcataacaagagaagtgggttttgtcgtgcctggtttgcctggcacgatattacgcacaactaatggtgactttttgcatttcttacctagagttttta |
| | | gtgcagttggtaacatctgttacacaccatcaaaacttatagagtacactgactttgcaacatcagcttgtgttttggctgctgaatgtacaattttta |
| | | aagatgcttctggtaagccagtaccatattgttatgataccaatgtactagaaggttctgttgcttatgaaagtttacgccctgacacacgttatgtgc |
| | | tcatggatggctctattattcaatttcctaacacctaccttgaaggttctgttagagtggtaacaacttttgattctgagtactgtaggcacggcactt |
| | | gtgaaagatcagaagctggtgtttgtgtatctactagtggtagatgggtacttaacaatgattattacagatctttaccaggagttttctgtggtgtag |
| | | atgctgtaaatttacttactaatatgtttacaccactaattcaacctattggtgctttggacatatcagcatctatagtagctggtggtattgtagcta |
| | | tcgtagtaacatgccttgcctactattttatgaggtttagaagagcttttggtgaatacagtcatgtagttgcctttaatactttactattccttatgt |
| | | cattcactgtactctgtttaacaccagtttactcattcttacctggtgtttattctgttatttacttgtacttgacattttatcttactaatgatgttt |
| | | cttttttagcacatattcagtggatggttatgttcacacctttagtacctttctggataacaattgcttatatcatttgtatttccacaaagcatttct |
| | | attggttattagtaattacctaaagagacgtgtagtctttaatggtgtttcctttagtacttttgaagaagctgcgctgtgcacctttttgttaaataa |
| | | agaaatgtatctaaagttgcgtagtgatgtgctattacctcttacgcaatataatagatacttagctctttataataagtacaagtattttagtggagc |
| | | aatggatacaactagctacagagaagctgcttgttgtcatctcgcaaaggctctcaatgacttcagtaactcaggttctgatgttattaccaaccacca |
| | | caaacctctatcacctcagctgttttgcagagtggttttagaaaaatggcattcccatctggtaaagttgagggttgtatggtacaagtaacttgtggt |
| | | acaactacacttaacggtctttggcttgatgacgtagtttactgtccaagacatgtgatctgcacctctgaagacatgcttaaccctaattatgaagat |
| | | ttactcattcgtaagtctaatcataatttcttggtacaggctggtaatgttcaactcagggttattggacattctatgcaaaattgtgtacttaagctt |
| | | aaggttgatacagccaatcctaagacacctaagtataagtttgttcgcattcaaccaggacagactttttcagtgttagcttgttacaatggttcacca |
| | | tctggtgtttaccaatgtgctatgaggcccaatttcactattaagggttcattccttaatggttcatgtggtagtgttggttttaacatagattatgac |
| | | tgtgtctctttttgttacatgcaccatatggaattaccaactggagttcatgctggcacagacttagaaggtaacttttatggaccttttgttgacagg |
| | | caaacagcacaagcagctggtacggacacaactattacagttaatgttttagcttggttgtacgctgctgttataaatggagacaggtggtttctcaat |
| | | cgatttaccacaactcttaatgactttaaccttgtggctatgaagtacaattatgaacctctaacacaagaccatgttgacatactaggacctctttct |
| | | gctcaaactggaattgccgttttagatatgtgtgcttcattaaaagaattactgcaaaatggtatgaatggacgtaccatattgggtagtgattattag |
| | | aagatgaatttacaccttttgatgttgttagacaatgctcaggtgttactttccaaagtgcagtgaaaagaacaatcaagggtacacaccactggttgt |
| | | tactcacaattttgacttcacttttagttttagtccagagtactcaatggtctttgttcttttttttgtatgaaaatgcctttttaccttttgctatgg |
| | | gtattattgctatgtctgcttttgcaatgatgtttgtcaaacataagcatgcatttactgtttgtttttgttaccttctcttgccactgtagcttattt |
| | | taatatggtctatatgcctgctagttgggtgatgcgtattatgacatggttggatatggttgatactagtttgtaggttttaagctaaaagactgtgtt |
| | | atgtatgcatcagctgtagtgttactaatccttatgacagcaagaactgtgtatgatgatggtgctaggagagtgtggacacttatgaatgtcttgaca |
| | | ctcgtttataaagtttattatggtaatgattagatcaagccatttccatgtgggctcttataatctctgttacttctaactactcaggtgtagttacaa |
| | | ctgtcatgtttttggccagaggtattgtttttatgtgtgttgagtattgccctattttcttcataactggtaatacacttcagtgtataatgctagttt |
| | | attgtttcttaggctatttttgtacttgttactttggcctcttttgtttactcaaccgctactttagactgactcttggtgtttatgattacttagttt |
| | | ctacacaggagtttagatatatgaattcacagggactactcccacccaagaatagcatagatgccttcaaactcaacattaaattgttgggtgttggtg |
| | | gcaaaccttgtatcaaagtagccactgtacagtctaaaatgtcagatgtaaagtgcacatcagtagtcttactctcagttttgcaacaactcagagtag |
| | | aatcatcatctaaattgtgggctcaatgtgtccagttacacaatgacattctcttagctaaagatactactgaagcctttgaaaaaatggtttcactac |
| | | tttctgttttgattccatgcagggtgctgtagacataaacaagctttgtgaagaaatgctggacaacagggcaaccttacaagctatagcctcagagtt |
| | | tagttcccttccatcatatgcagcttttgctactgctcaagaagcttatgagcaggctgttgctaatggtgattctgaagttgttcttaaaaagttgaa |
| | | gaagtctttgaatgtggctaaatctgaatttgaccgtgatgcagccatgcaacgtaagttggaaaagatggctgatcaagctatgacccaaatgtataa |
| | | acaggctagatctgaggacaagagggcaaaagttactagtgctatgcagacaatgatttcactatgcttagaaagttggataatgatgcactcaacaac |
| | | attatcaacaatgcaagagatggttgtgttcccttgaacataatacctcttacaacagcagccaaactaatggttgtcataccagactataacacatat |
| | | aaaaatacgtgtgatggtacaacatttacttatgcatcagcattgtgggaaatccaacaggttgtagatgcagatagtaaaattgttcaacttagtgaa |
| | | attagtatggacaattcacctaatttagcatggcctcttattgtaacagctttaagggccaattctgctgtcaaattacagaataatgagcttagtcct |
| | | gttgcactacgacagatgtcttgtgctgccggtactacacaaactgcttgcactgatgacaatgcgttagcttactacaacacaacaaagggaggtagg |
| | | tttgtacttgcactgttatccgatttacaggatttgaaatgggctagattccctaagagtgatggaactggtactatctatacagaactggaaccacct |
| | | tgtaggtttgttacagacacacctaaaggtcctaaagtgaagtatttatactttattaaaggattaaacaacctaaatagaggtatggtacttggtagt |
| | | ttagctgccacagtacgtctacaagctggtaatgcaacagaagtgcctgccaattcaactgtattatattctgtgatttgctgtagatgctgctaaagc |
| | | ttacaaagattatctagctagtgggggacaaccaatcactaattgtgttaagatgttgtgtacacacactggtactggtcaggcaataacagttacacc |
| | | ggaagccaatatggatcaagaatcctttggtggtgcatcgtgttgtctgtactgccgttgccacatagatcatccaaatcctaaaggattttgtgactt |
| | | aaaaggtaagtatgtacaaatacctacaacttgtgctaatgaccagtgggttttacacttaaaaacacagtagtaccgtctgcggtatgtggaaaggtt |
| | | atggctgtagttgtgatcaactccgcgaacccatgcttcagtcagctgatgcacaatcgtttttaaacgggtttgcggtgtaagtgcagcccgtcttac |
| | | accgtgcggcacaggcactagtactgatgtcgtatacagggcttttgacatctacaatgataaagtagaggttttgctaaattcctaaaaactaattgt |
| | | tgtcgcttccaagaaaaggacgaagatgacaatttaattgattcttactttgtagttaagagacacactttctctaactaccaacatgaagaaacaatt |
| | | tataatttacttaaggattgtccagctgttgctaaacatgacttctttaagtttagaatagacggtgacatggtaccacatatatcacgtcaacgtctt |
| | | actaaatacacaatggcagacctcgtctatgctttaaggcattttgatgaaggtaattgtgacacattaaaagaaatacttgtcacatacaattgttgt |
| | | gatgatgattatttcaataaaaaggactggtatgattttgtagaaaacccagatatattacgcgtatacgccaacttaggtgaacgtgtacgccaagat |
| | | tgttaaaaacagtacaattctgtgatgccatgcgaaatgctggtattgttggtgtactgacattagataatcaagatctcaatggtaactggtatgatt |
| | | tcggtgatttcatacaaaccacgccaggtagtggagttcctgttgtagattcttattattcattgttaatgcctatattaaccttgaccagggctttaa |
| | | ctgcagagtcacatgttgacactgacttaacaaagccttacattaagtgggatttgttaaaatatgacttcacggaagagaggttaaaactattgaccg |
| | | ttattttaaatattgggatcagacataccacccaaattgtgttaactgtttggatgacagatgcattctgcattgtgcaaactttaatgttttattact |
| | | acagtgttcccacctacaagttttggaccactagtgagaaaaatatttgttgatggtgttccatttgtagtttcaactggataccacttcagagagcta |
| | | ggtgttgtacataatcaggatgtaaacttacatagactagacttagttttaaggaattacttgtgtatgctgctgaccctgctatgcacgctgatctgg |
| | | taatctattactagataaacgcactacgtgcttttcagtagctgcacttactaacaatgttgatttcaaactgtcaaacccggtaattttaacaaagac |
| | | ttctatgactttgctgtgtctaagggtttattaaggaaggaagttctgttgaattaaaacacttcttattgctcaggatggtaatgctgctatcagcga |
| | | ttatgactactatcgttataatctaccaacaatgtgtgatatcagacaactactatttgtagttgaagttgttgataagtactttgattgttacgatgg |
| | | tggctgtattaatgctaaccaagtcatcgtcaacaacctagacaaatcagaggttttccatttaataaatggggtaaggctagactttattatgattca |
| | | atgagttatgaggatcaagatgcacttttcgcatatacaaaacgtaatgtcatccctactataactcaaatgaatcttaagtatgccattagtgcaaag |
| | | aatagagctcgcaccgtagctggtgtctctatctgtagtactatgaccaatagacagtttcatcaaaaattattgaaatcaatagccgccactagagga |
| | | gctactgtagtaattggaacaagcaaattctatggtggttggcacaacatgttaaaaactgtttatagtgatgtagaaaaccdcaccttatgggttggg |
| | | attatcctaaatgtgatagagccatgcctaacatgcttagaattatggcctcacttgttcttgctcgcaaacatacaacgtgttgtagcttgtcacacc |
| | | gtttctatagattagctaatgagtgtgctcaagtattgagtgaaatggtcatgtgtggcggttcactatatgttaaaccaggtggaacctcatcaggag |
| | | atgccacaactgcttatgctaatagtgtttttaacatttgtcaagctgtcacggccaatgttaatgcacttttatctactgatggtaacaaaattgccg |
| | | ataagtatgtccgcaatttacaacacagactttatgagtgtctctatagaaatagagatgttgacacagactttgtgaatgagttttacgcatatttgc |
| | | gtaaacatttctcaatgatgatactctctgacgatgctgttgtgtgtttcaatagcacttatgcatctcaaggtctagtggctagcataaagaacttta |
| | | agtcagttattattatcaaaacaatgtttttatgtctgaagcaaaatgttggactgagactgaccttactaaaggacctcatgaattttgactcaacat |
| | | acaatgctagttaaacagggtgatgattatgtgtaccttccttacccagatccatcaagaatcctaggggccggctgttttgtagatgatatcgtaaaa |
| | | acagatggtacacttatgattgaacggttcgtgtctttagctatagatgcttacccacttactaaacatcctaatcaggagtatgctgatgtattcatt |
| | | tgtacttacaatacataagaaagctacatgatgagttaacaggacacatgttagacatgtattctgttatgcttactaatgataacacttcaaggtatt |
| | | gggaacctgagttttatgaggctatgtacacaccgcatacagtcttacaggctgttggggcttgtgttattgcaattcacagacttcattaagatgtgg |
| | | tgcttgcatacgtagaccattcttatgttgtaaatgctgttacgaccatgtcatatcaacatcacataaattagtcttgtagttaatccgtatgtttgc |
| | | aatgctccaggttgtgatgtcacagatgtgactcaactttacttaggaggtatgagctattattgtaaatcacataaaccacccattagttttccattg |
| | | tgtgctaatggacaagtttttggtttatataaaaatacatgtgttggtagcgataatgttactgactttaatgcaattgcaacatgtgactggacaaat |
| | | gctggtgattacattttagctaacacctgtactgaaagactcaagattttgcagcagaaacgctcaaagctactgaggagacatttaaactgtcttatg |
| | | gtattgctactgtacgtgaagtgctgtctgacagagaattacatctttcatgggaagttggtaaacctagaccaccacttaaccgaaattatgtattac |
| | | tggttatcgtgtaactaaaaacagtaaagtacaaataggagagtacacctttgaaaaaggtgactatggtgatgctgttgtttaccgaggtacaacaac |
| | | ttacaaattaaatgttggtgattattttgtgctgacatcacatacagtaatgccattaagtgcacctacactagtgccacaagagcactatgttagaat |
| | | tactggcttatacccaacactcaatatctcagatgagttttctagcaatgttgcaaattatcaaaaggttggtatgcaaaagtattctacactccaggg |
| | | accacctggtactggtaagagtcattttgctattggcctagctctctactacccttctgctcgcatagtgtatacagcttgactcatgccgctgttgat |
| | | gcactatgtgagaaggcattaaaatatttgcctatagataaatgtagtagaattatacctgcacgtgctcgtgtagagtgttttgataaattcaaagtg |
| | | aattcaacattagaacagtatgtatttgtactgtaaatgcattgcctgagacgacagcagatatagttgtattgatgaaatttcaatggccacaaatta |
| | | tgatttgagtgttgtcaatgccagattacgtgctaagcactatgtgtacattggcgaccctgctcaattacctgcaccacgcacattgctaactaaggg |
| | | cacactagaaccagaatatttcaattcagtgtgtagacttatgaaaactataggtccagacatgttcctcggaacttgtcggcgttgtcctgctgaaat |
| | | tgttgacactgtgagtgctttggtttatgataataagcttaaagcacataaagacaaatcagctcaatgattaaaatgttttataagggtgttatcacg |
| | | catgatgtttcatctgcaattaacaggccacaaataggcgtggtaagagaattccttacacgtaaccctgcttggagaaaagctgtctttatttcacct |
| | | tataattcacagaatgctgtagcctcaaagattttgggactaccaactcaaactgttgattcatcacagggctcagaatatgactatgtcatattcact |
| | | caaaccactgaaacagctcactcttgtaatgtaaacagatttaatgttgctattaccagagcaaaagtaggcatactttgcataatgtctgatagagac |
| | | ctttatgacaagttgcaatttacaagtcttgaaattccacgtaggaatgtggcaactttacaagctgaaaatgtaacaggactctttaaagattgtagt |
| | | aaggtaatcactgggttacatcctacacaggcacctacacacctcagtgttgacactaaattcaaaactgaaggtttatgtgttgacatacctggcata |
| | | cctaaggacatgacctatagaagactcatctctatgatgggttttaaaatgaattatcaagttaatggttaccctaacatgtttatcacccgcgaagaa |
| | | gctataagacatgtacgtgcatggattggcttcgatgtcgaggggtgtcatgctactagagaagctgttggtaccaatttacctttacagctaggtttt |
| | | tctacaggtgttaacctagttgctgtacctacaggttatgttgatacacctaataatacagatttttccagagttagtgctaaaccaccgcctggagat |
| | | caatttaaacacctcataccacttatgtacaaaggacttccttggaatgtagtgcgtataaagattgtacaaatgttaagtgacacacttaaaaatctc |
| | | tctgacagagtcgtatttgtcttatgggcacatggctttgagttgacatctatgaagtattttgtgaaaataggacctgagcgcacctgttgtctatgt |
| | | gatagacgtgccacatgatttccactgcttcagacacttatgcctgttggcatcattctattggatttgattacgtctataatccgtttatgattgatg |
| | | ttcaacaatggggttttacaggtaacctacaaagcaaccatgatctgtattgtcaagtccatggtaatgcacatgtagctagttgtgatgcaatcatga |
| | | ctaggtgtctagagtccacgagtgctttgttaagcgtgttgactggactattgaatatcctataattggtgatgaactgaagattaatgcggcttgtag |
| | | aaaggttcaacacatggttgttaaagctgcattattagcagacaaattcccagttcttcacgacattggtaaccctaaagctattaagtgtgtacctca |
| | | agctgatgtagaatggaagttctatgatgcacagccttgtagtgacaaagcttataaaatagaagaattattctattcttatgccacacattctgacaa |
| | | attcacagatggtgtatgcctattttggaattgcaatgtcgatagatatcctgctaattccattgtttgtagatttgacactagagtgctatctaacct |
| | | taacttgcctggttgtgatggtggcagtttgtatgtaaataaacatgcattccacacaccagcttttgataaaagtgatttgttaatttaaaacaatta |
| | | ccatttttctattactctgacagtccatgtgagtctcatggaaaacaagtagtgtcagatatagattatgtaccactaaagtctgctacgtgtataaca |
| | | cgttgcaatttaggtggtgctgtctgtagacatcatgctaatgagtacagattgtatctcgatgcttataacatgatgatctcagctggctttagcttg |
| | | tgggtttacaaacaatttgatacttataacctctggaacacttttacaagacttcagagtttagaaaatgtggcttttaatgttgtaaataagggacac |
| | | tttgatggacaacagggtgaagtaccagtttctatcattaataacactgtttacacaaaagttgatggtgttgatgtagaattgtttgaaaataaaaca |
| | | acattacctgttaatgtagcatttgagctttgggctaagcgcaacattaaaccagtaccagaggtgaaaatactcaataatttgggtgtggacattgct |
| | | gctaatactgtgatctgggactacaaaagagatgaccagcacatatatctactattggtgtttgttctatgactgacatagccaagaaaccaactgaaa |
| | | cgatttgtgcaccactcactgtcttttttgatggtagagttgatggtcaagtagacttatttagaaatgcccgtaatggtgttcttattacagaaggta |
| | | gtgttaaaggtttacaaccatctgtaggtcccaaacaagctagtcttaatggagtcacattaattggagaagccgtaaaaacacagttcaattattata |
| | | agaaagttgatggtgttgtccaacaattacctgaaacttactttactcagagtagaaatttacaagaatttaaacccaggagtcaaatggaaattgatt |
| | | tcttagaattagctatggatgaattcattgaacggtataaattagaaggctatgccttcgaacatatcgtttatggagattttagtcatagtcagttag |
| | | gtggtttacatctactgattggactagctaaacgttttaaggaatcaccttttgaattagaagattttattcctatggacagtacagttaaaaactatt |
| | | tcataacagatgcgcaaacaggttcatctaagtgtgtgtgttctgttattgatttattacttgatgattttgttgaaataataaaatcccaagatttat |
| | | ctgtagtttctaaggttgtcaaagtgactattgactatacagaaatttcatttatgattggtgtaaagatggccatgtagaaacatt-ttacccaaaat |
| | | tacaatctagtcaagcgtggcaaccgggtgttgctatgcctaatattacaaaatgcaaagaatgctattagaaaagtgtgaccttcaaaattatggtga |
| | | tagtgcaacattacctaaaggcataatgatgaatgtcgcaaaatatactcaactgtgtcaatatttaaacacattaacattagctgtaccctataatat |
| | | gagagttatacattttggtgctggttctgataaaggagttgcaccaggtacagctgttttaagacagtggttgcctacgggtacgctgcttgtcgattc |
| | | agatcttaatgactttgtctctgatgcagattcaactttgattggtgattgtgcaactgtacatacagctaataaatgggatctcattattagtgatat |
| | | gtacgaccctaagactaaaaatgttacaaaagaaaatgactctaaagagggttttttcacttacatttgtgggtttatacaacaaaagctagctcttgg |
| | | aggttccgtggctataaagataacagaacattcttggaatgctgatctttataagctcatgggacacttcgcatggtggacagcctttgttactaatgt |
| | | gaatgcgtcatcatctgaagcatttttaattggatgtaattatcttggcaaaccacgcgaacaaatagatggttatgtcatgcatgcaaattacatatt |
| | | ttggaggaatacaaatccaattcagttgtcttcctattctttatttgacatgagtaaatttccccttaaattaaggggtactgctgttatgtattaaaa |
| | | gaaggtcaaatcaatgatatgattttatctcttcttagtaaaggtagacttataattagagaaaacaacagagttgttatttctagtgatgttcttgtt |
| | | aacaactaaacgaacaatgtttgtttttcttgttttattgccactagtctctagtcagtgtgttaatcttacaaccagaactcaattaccccctgcata |
| | | cactaattattcacacgtggtgtttattaccctgacaaagttttcagatcctcagttttacattcaactcaggacttgttcttacctttcttttccaat |
| | | gttacttggttccatgctatacatgtctctgggaccaatggtactaagaggtttgataaccagtcctaccatttaatgatggtgtttattttgcttcca |
| | | ctgagaagtctaacataataagaggctggatttttggtactactttagattcgaagacccagtccctacttattgttaataacgctactaatgttgtta |
| | | ttaaagtagtgaatttcaattttgtaatgatccatttttgggtgtttattaccacaaaaacaacaaaagttggatggaaagtgagttcagagtttattc |
| | | tagtgcgaataattgcacttttgaatatgtctctcagccttttcttatggaccttgaaggaaaacagggtaatttcaaaaatcttagggaatttgtgtt |
| | | taagaatattgatggttattttaaaatatattctaagcacacgcctattaatttagtgcgtgatctccctcagggtttttcggctttagaaccattggt |
| | | agatttgccaataggtattaacatcactaggtttcaaactttacttgctttacatagaagttatttgactcctggtgattcttcttcaggttggacaga |
| | | ggtgctgcagcttattatgtgggttatcttcaacctaggacttttctattaaaatataatgaaaatggaaccattacagatgctgtagactgtgcactt |
| | | gaccctctctcagaaacaaagtgtacgttgaaatccttcactgtagaaaaaggaatctatcaaacttctaactttagagtccaaccaacagaatctatt |
| | | gttagatttcctaatattacaaacttgtgccatttggtgaagtttttaacgccaccagatttgcatctgtttatgcttggaacaggaagagaatcagca |
| | | actgtgttgctgattattctgtcctatataattccgcatcattttccacttttaagtgttatggagtgtctcctactaaattaaatgatctctgattac |
| | | taatgtctatgcagattcatttgtaattagaggtgatgaagtcagacaaatcgctccagggcaaactggaaagattgctgattataattataaattacc |
| | | agatgattttacaggctgcgttatagcttggaattctaacaatcttgattctaaggttggtggtaattataattacctgtatagattgtttaggaagtc |
| | | taatctcaaaccttttgagagagatatttcaactgaaatctatcaggccggtagcacaccttgtaatggtgttgaaggttttaattgttactttcatta |
| | | caatcatatggtttccaacccactaatggtgttggttaccaaccatacagagtagtagtactttatttgaacttctacatgcaccagcaactgtttgtg |
| | | gacctaaaaagtctactaatttggttaaaaacaaatgtgtcaatttcaacttcaatggtttaacaggcacaggtgttcttactgagtctaacaaaaagt |
| | | ttctgcctttccaacaatttggcagagacattgctgacactactgatgctgtccgtgatccacagacacttgagattcttgacattacaccatgttatt |
| | | tggtggtgtcagtgttataacaccaggaacaaatacttctaaccaggttgctgttctttatcaggatgttaactgcacagaagtccctgttgctattca |
| | | tgcagatcaacttactcctacttggcgtgtttattctacaggttctaatgtttttcaaacacgtgcaggctgtttaataggggctgaacatgtcaacaa |
| | | ctcatatgagtgtgacatacccattggtgcaggtatatgcgctagttatcagactcagactaattctcctcggcgggcacgtagtgtagctagtcaatc |
| | | catcattgcctacactatgtcacttggtgcagaaaattcagttgcttactctaataactctattgccatacccacaaattttactattagtgttaccac |
| | | agaaattctaccagtgtctatgaccaagacatcagtagattgtacaatgtacatttgtggtgattcaactgaatgcagcaatatttgttgcaatatggc |
| | | agtttttgtacacaattaaaccgtgattaactggaatagagttgaacaagacaaaaacacccaagaagtttttgcacaagtcaaacaaatttacaaaac |
| | | accaccaattaaagattttggtggttttaatttttcacaaatattaccagatccatcaaaaccaagcaagaggtcatttattgaagatctacttttcaa |
| | | caaagtgacacttgcagatgctggcttcatcaaacaatatggtgattgccttggtgatattgctgctagagacctcatttgtgcacaaaagtttaacgg |
| | | ccttactgttttgccacctttgctcacagatgaaatgattgctcaatacacttctgcactgttagcgggtacaatcacttctggttggacctttggtgc |
| | | aggtgctgcattacaaataccatttgctatgcaaatggcttataggtttaatggtattggagttacacagaatgttactatgagaaccaaaaattgatt |
| | | gccaaccaatttaatagtgctattggcaaaattcaagactcactttcttccacagcaagtgcacttggaaaacttcaagatgtggtcaaccaaaatgca |
| | | caagctttaaacacgcttgttaaacaacttagctccaattttggtgcaatttcaagtgttttaaatgatatcctttcacgtcttgacaaagttgaggct |
| | | gaagtgcaaattgataggttgatcacaggcagacttcaaagtttgcagacatatgtgactcaacaattaattagagctgcagaaatcagagcttctgct |
| | | aatcttgctgctactaaaatgtcagagtgtgtacttggacaatcaaaaagagttgatttttgtggaaagggctatcatcttatgtccttccctcagtca |
| | | gcacctcatggtgtagtcttcttgcatgtgacttatgtccctgcacaagaaaagaacttcacaactgctcctgccatttgtcatgatggaaaagcacac |
| | | tttcctcgtgaaggtgtctttgtttcaaatggcacacactggtttgtaacacaaaggaatttttatgaaccacaaatcattactacagacaacacattt |
| | | gtgtaggtaactgtgatgttgtaataggaattgtcaacaacacagtttatgatcattgcaacctgaattagactcattcaaggaggagttagataaata |
| | | ttttaagaatcatacatcaccagatgttgatttaggtgacatctctggcattaatgcttcagttgtaaacattcaaaaagaaattgaccgcctcaatga |
| | | ggttgccaagaatttaaatgaatctctcatcgatctccaagaacttggaaagtatgagcagtatataaaatggccatggtacatttggctaggttttat |
| | | agaggcttgattgccatagtaatggtgacaattatgattgctgtatgaccagttgctgtagttgtctcaagggctgttgttcttgtggatcctgctgca |
| | | aatttgatgaagacgactctgagccagtgctcaaaggagtcaaattacattacacataaacgaacttatggatttgtttatgagaatcttcacaattgg |
| | | aactgtaactttgaagcaaggtgaaatcaaggatgctactccttcagattttgttcgcgctactgcaacgataccgatacaagcctcactccctttcgg |
| | | atggcttattgttggcgttgcacttcttgctgtttttcagagcgcttccaaaatcataaccctcaaaaagagatggcaactagcactctccaagggtgt |
| | | tcactttgtttgcaacttgctgttgttgtttgtaacagtttactcacaccttttgctcgttgctgctggccttgaagccccttttctctatctttatga |
| | | ttagtctacttatgcagagtataaactttgtaagaataataatgaggctttggctttgctggaaatgccgttccaaaaacccattactttatgatgcca |
| | | actattttctttgctggcatactaattgttacgactattgtataccttacaatagtgtaacttcttcaattgtcattacttcaggtgatggcacaacaa |
| | | gtcctatttctgaacatgactaccagattggtggttatactgaaaaatgggaatctggagtaaaagactgtgttgtattacacagttacttcacttcag |
| | | actattaccagctgtactcaactcaattgagtacagacactggtgttgaacatgttaccttcttcatctacaataaaattgttgatgagcctgaagaac |
| | | atgtccaaattcacacaatcgacggttcatccggagttgttaatccagtaatggaaccaatttatgatgaaccgacgacgactactagcgtgcctttgt |
| | | aagcacaagctgatgagtacgaacttatgtactcattcgtttcggaagagacaggtacgttaatagttaatagcgtacttctttttcttgattcgtggt |
| | | attcttgctagttacactagccatccttactgcgcttcgattgtgtgcgtactgctgcaatattgttaacgtgagtcttgtaaaaccttctttttacgt |
| | | ttactctcgtgttaaaaatctgaattcttctagagttcctgatcttctggtctaaacgaactaaatattatattagtttttctgtttggaactttaatt |
| | | ttagccatggcagattccaacggtactattaccgttgaagagcttaaaaagctccttgaacaatggaacctagtaataggtttcctattccttacatgg |
| | | atttgtcttctacaatttgcctatgccaacaggaataggtttttgtatataattaagttaattttcctaggctgttatggccagtaactttagcttgtt |
| | | ttgtgcttgctgctgtttacagaataaattggatcaccggtggaattgctatcgcaatggcttgtcttgtaggcttgatgtggctcagctacttcattg |
| | | cttctttcagactgtttgcgcgtacgcgttccatgtggtcattcaatccagaaactaacattcttctcaacgtgccactccatggcactattctgacca |
| | | gaccgcttctagaaagtgaactcgtaatcggagctgtgatccttcgtggacatcttcgtattgctggacaccatctaggacgctgtgacatcaaggacc |
| | | tgcctaaagaaatcactgttgctacatcacgaacgctttcttattacaaattgggagcttcgcagcgtgtagcaggtgactcaggttttgctgcataca |
| | | gtcgctacaggattggcaactataaattaaacacagaccattccagtagcagtgacaatattgattgcttgtacagtaagtgacaacagatgtttcatc |
| | | tcgttgactttcaggttactatagcagagatattactaattattatgaggacttttaaagtttccatttggaatcttgattacatcataaacctcataa |
| | | ttaaaaatttatctaagtcactaactgagaataaatattctcaattagatgaagagcaaccaatggagattgattaaacgaacatgaaaattattattt |
| | | cttggcactgataacactcgctacttgtgagattatcactaccaagagtgtgttagaggtacaacagtacttttaaaagaaccttgctcttctggaaca |
| | | tacgagggcaattcaccatttcatcctctagctgataacaaatttgcactgacttgctttagcactcaatttgcttttgcttgtcctgacggcgtaaaa |
| | | cacgtctatcagttacgtgccagatcagtttcacctaaactgttcatcagacaagaggaagttcaagaactttactctccaatttttcttattgttgcg |
| | | gcaatagtgtttataacactttgcttcacactcaaaagaaagacagaatgattgaactttcattaattgacttctatttgtgctttttagcctttctgc |
| | | tattccttgttttaattatgcttattatcttttggttctcacttgaactgcaagatcataatgaaacttgtcacgcctaaacgaacatgaaatttcttg |
| | | ttttcttaggaatcatcacaactgtagctgcatttcaccaagaatgtagtttacagtcatgtactcaacatcaaccatatgtagttgatgacccgtgtc |
| | | ctattcacttctattctaaatggtatattagagtaggagctagaaaatcagcacctttaattgaattgtgcgtggatgaggctggttctaaatcaccca |
| | | ttcagtacatcgatatcggtaattatacagtttcctgtttaccttttacaattaattgccaggaacctaaattgggtagtcttgtagtgcgttgttcgt |
| | | tctatgaagactttttagagtatcatgacgttcgtgttgttttagatttcatctaaacgaacaaactaaaatgtctgataatggaccccaaaatcagcg |
| | | aaatgcaccccgcattacgtttggtggaccctcagattcaactggcagtaaccagaatggagaacgcagtggggcgcgatcaaaacaacgtcggcccca |
| | | aggtttacccaataatactgcgtatggttcaccgctctcactcaacatggcaaggaagaccttaaattccctcgaggacaaggcgttccaattaacacc |
| | | aatagcagtccagatgaccaaattggctactaccgaagagctaccagacgaattcgtggtggtgacggtaaaatgaaagatctcagtccaagatggtat |
| | | ttctactacctaggaactgggccagaagctggacttccctatggtgctaacaaagacggcatcatatgggttgcaactgagggagccttgaatacacca |
| | | aaagatcacattggcacccgcaatcctgctaacaatgctgcaatcgtgctacaacttcctcaaggaacaacattgccaaaaggcttctacgcagaaggg |
| | | agcagaggcggcagtcaagcctcttctcgttcctcatcacgtagtcgcaacagttcaagaaattcaactccaggcagcagtaggggaacttctcctgct |
| | | agaatggctggcaatggcggtgatgctgctcttgctttgctgctgcttgacagattgaaccagcttgagagcaaaatgtctggtaaaggccaacaacaa |
| | | caaggccaaactgtcactaagaaatctgctgctgaggcttctaagaagcctcggcaaaaacgtactgccactaaagcatacaatgtaacacaagctttc |
| | | ggcagacgtggtccagaacaaacccaaggaaattttggggaccaggaactaatcagacaaggaactgattacaaacattggccgcaaattgcacaattt |
| | | gcccccagcgcttcagcgttcttcggaatgtcgcgcattggcatggaagtcacaccttcgggaacgtggttgacctacacaggtgccatcaaattggat |
| | | gacaaagatccaaatttcaaagatcaagtcattttgctgaataagcatattgacgcatacaaaacattcccaccaacagagcctaaaaaggacaaaaag |
| | | aagaaggctgatgaaactcaagccttaccgcagagacagaagaaacagcaaactgtgactatatcctgctgcagatttggatgatttctccaaacaatt |
| | | gcaacaatccatgagcagtgctgactcaactcaggcctaaactcatgcagaccacacaaggcagatgggctatataaacgttttcgcttttccgtttac |
| | | gatatatagtctactcttgtgcagaatgaattctcgtaactacatagcacaagtagatgtagttaactttaatctcacatagcaatctttaatcagtgt |
| | | gtaacattagggaggacttgaaagagccaccacattttcaccgaggccacgcggagtacgatcgagtgtacagtgaacaatgctagggagagctgccta |
| | | tatggaagagccctaatgtgtaaaattaattttagtagtgctatccccatgtgattttaatagatataggagaatgacaaaaaaaaaaaaaaaaaaaaa |
| | | aaaaaaaaaaaa |
| 5536 | SARS-CoV-1 genome | atattaggtttttacctacccaggaaaagccaaccaacctcgatctcttgtagatctgttctctaaacgaactttaaaatctgtgtagctgtcgctcgg |
| | Genbank Accession No. NC_004718.3 | ctgcatgcctagtgcacctacgcagtataaacaataataaattttactgtcgttgacaagaaacgagtaactcgtccctcttctgcagactgcttacgg |
| | | tttcgtccgtgttgcagtcgatcatcagcatacctaggtttcgtccgggtgtgaccgaaaggtaagatggagagccttgttcttggtgtcaacgagaaa |
| | | acacacgtccaactcagtttgcctgtccttcaggttagagacgtgctagtgcgtggcttcggggactctgtggaagaggccctatcggaggcacgtgaa |
| | | cacctcaaaaatggcacttgtggtctagtagagctggaaaaaggcgtactgccccagcttgaacagccctatgtgttcattaaacgttctgatgcctta |
| | | agcaccaatcacggccacaaggtcgttgagctggttgcagaaatggacggcattcagtacggtcgtagcggtataacactgggagtactcgtgccacat |
| | | gtgggcgaaaccccaattgcataccgcaatgttcttcttcgtaagaacggtaataagggagccggtggtcatagctatggcatcgatctaaagtcttat |
| | | gacttaggtgacgagcttggcactgatcccattgaagattatgaacaaaactggaacactaagcatggcagtggtgcactccgtgaactcactcgtgag |
| | | ctcaatggaggtgcagtcactcgctatgtcgacaacaatttctgtggcccagatgggtaccctcttgattgcatcaaagattttctcgcacgcgcgggc |
| | | aagtcaatgtgcactattccgaacaacttgattacatcgagtcgaagagaggtgtctactgctgccgtgaccatgagcatgaaattgcctggttcactg |
| | | agcgctctgataagagctacgagcaccagacacccttcgaaattaagagtgccaagaaatttgacactttcaaaggggaatgcccaaagtttgtgtttc |
| | | ctcttaactcaaaagtcaaagtcattcaaccacgtgttgaaaagaaaaagactgagggtttcatggggcgtatacgctctgtgtaccctgttgcatctc |
| | | cacaggagtgtaacaatatgcacttgtctaccttgatgaaatgtaatcattgcgatgaagtttcatggcagacgtgcgactttctgaaagccacttgtg |
| | | aacattgtggcactgaaaatttagttattgaaggacctactacatgtgggtacctacctactaatgctgtagtgaaaatgccatgtcctgcctgtcaag |
| | | acccagagattggacctgagcatagtgttgcagattatcacaaccactcaaacattgaaactcgactccgcaagggaggtaggactagatgttttggag |
| | | gctgtgtgtttgcctatgttggctgctataataagcgtgcctactgggttcctcgtgctagtgctgatattggctcaggccatactggcattactggtg |
| | | acaatgtggagaccttgaatgaggatctccttgagatactgagtcgtgaacgtgttaacattaacattgttggcgattttcatttgaatgaagaggttg |
| | | ccatcattttggcatctttctctgatctacaagtgcctttattgacactataaagagtcttgattacaagtattcaaaaccattgttgagtcctgcggt |
| | | aactataaagttaccaagggaaagcccgtaaaaggtgcttggaacattggacaacagagatcagttttaacaccactgtgtggttttccctcacaggct |
| | | gctggtgttatcagatcaatttttgcgcgcacacttgatgcagcaaaccactcaattcctgatttgcaaagagcagctgtcaccatacttgatggtatt |
| | | tctgaacagtcattacgtcttgtcgacgccatggtttatacttcagacctgctcaccaacagtgtcattattatggcatatgtaactggtggtcttgta |
| | | caacagacttctcagtggttgtctaatcttttgggcactactgttgaaaaactcaggcctatctttgaatggattgaggcgaaacttagtgcaggagtt |
| | | gaatttctcaaggatgcttgggagattctcaaatttctcattacaggtgtttttgacatcgtcaagggtcaaatacaggttgcttcagataacatcaag |
| | | gattgtgtaaaatgcttcattgatgttgttaacaaggcactcgaaatgtgcattgatcaagtcactatcgctggcgcaaagttgcgatcactcaactta |
| | | ggtgaagtcttcatcgctcaaagcaagggactttaccgtcagtgtatacgtggcaaggagcagctgcaactactcatgcctcttaaggcaccaaaagaa |
| | | gtaacctttcttgaaggtgattcacatgacacagtacttacctctgaggaggttgttctcaagaacggtgaactcgaagcactcgagacgcccgttgat |
| | | agcttcacaaatggagctatcgttggcacaccagtctgtgtaaatggcctcatgctcttagagattaaggacaaagaacaatactgcgcattgtctcct |
| | | ggtttactggctacaaacaatgtctttcgcttaaaagggggtgcaccaattaaaggtgtaacctttggagaagatactgtttgggaagttcaaggttac |
| | | aagaatgtgagaatcacatttgagcttgatgaacgtgttgacaaagtgcttaatgaaaagtgctctgtctacactgttgaatccggtaccgaagttact |
| | | gagtttgcatgtgttgtagcagaggctgttgtgaagactttacaaccagtttctgatctccttaccaacatgggtattgatcttgatgagtggagtgta |
| | | gctacattctacttatttgatgatgctggtgaagaaaacttttcatcacgtatgtattgttcdtttaccctccagatgaggaagaagaggacgatgcag |
| | | agtgtgaggaagaagaaattgatgaaacctgtgaacatgagtacggtacagaggatgattatcaaggtaccactggaatttggtgcctcagctgaaaca |
| | | gttcgagttgaggaagaagaagaggaagactggctggatgatactactgagcaatcagagattgagccagaaccagaacctacacctgaagaaccagtt |
| | | aatcagtttactggttatttaaaacttactgacaatgttgccattaaatgtgttgacatcgttaaggaggcacaaagtgctaatcctatggtgattgta |
| | | aatgctgctaacatacacctgaaacatggtggtggtgtagcaggtgcactcaacaaggcaaccaatggtgccatgcaaaaggagagtgatgattacatt |
| | | aagctaaatggccacttacagtaggagggtcttgtttgattctggacataatcttgctaagaagtgtctgcatgttgttggacctaacctaaatgcagg |
| | | tgaggacatccagcttcttaaggcagcatatgaaaatttcaattcacaggacatcttacttgcaccattgttgtcagcaggcatatttggtgctaaacc |
| | | acttcagtattacaagtgtgcgtgcagacggttcgtacacaggtttatattgcagtcaatgacaaagctattatgagcaggttgtcatggattatcttg |
| | | ataacctgaagcctagagtggaagcacctaaacaagaggagccaccaaacacagaagattccaaaactgaggagaaatctgtcgtacagaagcctgtcg |
| | | atgtgaagccaaaaattaaggcctgcattgatgaggttaccacaacactggaagaaactaagtttcttaccaataagttactcttgtttgctgatatca |
| | | atggtaagattaccatgattctcagaacatgcttagaggtgaagatatgtattccttgagaaggatgcaccttacatggtaggtgatgttatcactagt |
| | | ggtgatatcacttgtgttgtaataccaccaaaaaggctggtggcactactgagatgctctcaagagctttgaagaaagtgccagttgatgagtatataa |
| | | ccacgtaccaggacaaggatgtgctggttatacacttgaggaagctaagactgctcttaagaaatgcaaatctgcattttatgtactaccttcagaagc |
| | | acctaatgctaaggaagagattctaggaactgtatcctggaatttgagagaaatgcttgctcatgctgaagagacaagaaaattaatgcctatatgcat |
| | | ggatgttagagccataatggcaaccatccaacgtaagtataaaggaattaaaattcaagagggcatcgttgactatggtgtccgattcttatttatact |
| | | agtaaagagcctgtagcttctattattacgaagctgaactctctaaatgagccgcttgtcacaatgccaattggttatgtgacacatggttttaatctt |
| | | gaagaggctgcgcgctgtatgcgttctcttaaagctcctgccgtagtgtcagtatcatcaccagatgctgttactacatataatggatacctcacttcg |
| | | tcatcaaagacatctgaggagcactttgtagaaacagtttctttggctggctcttacagagattggtcctattcaggacagcgtacagagttaggtgtt |
| | | gaatttcttaagcgtggtgacaaaattgtgtaccacactaggagagccccgtcgagtttcatcttgacggtgaggttattcacttgacaaactaaagag |
| | | tctcttatccctgcgggaggttaagactataaaagtgttcacaactgtggacaacactaatctccacacacagcttgtggatatgtctatgacatatgg |
| | | acagcagtttggtccaacatacttggatggtgctgatgttacaaaaattaaacctcatgtaaatcatgagggtaagactttattgtactacctagtgat |
| | | gacacactacgtagtgaagctttcgagtactaccatactcttgatgagagttttcttggtaggtacatgtctgattaaaccacacaaagaaatggaaat |
| | | ttcctcaagttggtggtttaacttcaattaaatgggctgataacaattgttatttgtctagtgttttattagcacttcaacagcttgaagtcaaattca |
| | | atgcaccagcacttcaagaggcttattatagagcccgtgctggtgatgctgctaacttttgtgcactcatactcgcttacagtaataaaactgttggcg |
| | | agcttggtgatgtcagagaaactatgacccatcttctacagcatgctaatttggaatctgcaaagcgagttcttaatgtggtgtgtaaacattgtggtc |
| | | agaaaactactaccttaacgggtgtagaagctgtgatgtatatgggtactctatcttatgataatcttaagacaggtgtttccattccatgtgtgtgtg |
| | | gtcgtgatgctacacaatatctagtacaacaagagtcttcttttgttatgatgtctgcaccacctgctgagtataaattacagcaaggtacattcttat |
| | | gtgcgaatgagtacactggtaactatcagtgtggtcattacactcatataactgctaaggagaccctctatcgtattgacggagctcaccttacaaaga |
| | | tgtcagagtacaaaggaccagtgactgatgttttctacaaggaaacatcttacactacaaccatcaagcctgtgtcgtataaactcgatggagttactt |
| | | acacagagattgaaccaaaattggatgggtattataaaaaggataatgcttactatacagagcagcctatagaccttgtaccaactcaaccattaccaa |
| | | atgcgagttttgataatttcaaactcacatgttctaacacaaaatttgctgatgatttaaatcaaatgacaggcttcacaaagccagcttcacgagagc |
| | | tatctgtcacattcttcccagacttgaatggcgatgtagtggctattgactatagacactattcagcgagtttcaagaaaggtgctaaattactgcata |
| | | agccaattgtttggcacattaaccaggctacaaccaagacaacgttcaaaccaaacacttggtgtttacgttgtctttggagtacaaagccagtagata |
| | | cttcaaattcatttgaagttctggcagtagaagacacacaaggaatggacaatcttgcttgtgaaagtcaacaacccacctctgaagaagtagtggaaa |
| | | atcctaccatacagaaggaagtcatagagtgtgacgtgaaaactaccgaagttgtaggcaatgtcatacttaaaccatcagatgaaggtgttaaagtaa |
| | | cacaagagttaggtcatgaggatcttatggctgcttatgtggaaaacacaagcattaccattaagaaacctaatgagctttcactagccttaggtttaa |
| | | aaacaattgccactcatggtattgctgcaattaatagtgttccttggagtaaaattttggcttatgtcaaaccattcttaggacaagcagcaattacaa |
| | | catcaaattgcgctaagagattagcacaacgtgtgtttaacaattatatgccttatgtgtttacattattgttccaattgtgtacttttactaaaagta |
| | | ccaattctagaattagagcttcactacctacaactattgctaaaaatagtgttaagagtgttgctaaattatgtttggatgccggcattaattatgtga |
| | | agtcacccaaattttctaaattgttcacaatcgctatgtggctattgttgttaagtatttgcttaggttactaatctgtgtaactgctgatttggtgta |
| | | ctcttatctaattttggtgctccttcttattgtaatggcgttagagaattgtatcttaattcgtctaacgttactactatggatttctgtgaaggttct |
| | | tttccttgcagcatttgtttaagtggattagactcccttgattcttatccagctcttgaaaccattcaggtgacgatttcatcgtacaagctagacttg |
| | | acaattttaggtaggccgctgagtgggttttggcatatatgttgttcacaaaattcttttatttattaggtctttcagctataatgcaggtgttctttg |
| | | gctattttgctagtcatttcatcagcaattcttggctcatgtggtttatcattagtattgtacaaatggcacccgtttctgcaatggttaggatgtaca |
| | | tcttctttgcttctttctactacatatggaagagctatgttcatatcatggatggttgcacctcttcgacttgcatgatgtgctataagcgcaatcgtg |
| | | ccacacgcgttgagtgtacaactattgttaatggcatgaagagatctttctatgtctatgcaaatggaggccgtggcttctgcaagactcacaattgga |
| | | attgtctcaattgtgacacattttgcactggtagtacattcattagtgatgaagttgctcgtgatttgtcactccagtttaaaagaccaatcaacccta |
| | | ctgaccagtcatcgtatattgttgatagtgttgctgtgaaaaatggcgcgcttcacctctactttgacaaggctggtcaaaagacctatgagagacatc |
| | | cgctctcccattttgtcaatttagacaatttgagagctaacaacactaaaggttcactgcctattaatgtcatagtttttgatggcaagtccaaatgcg |
| | | acgagtctgcttctaagtctgcttctgtgtactacagtcagctgatgtgccaacctattctgttgcttgaccaagctcttgtatcagacgttggagata |
| | | gtactgaagtttccgttaagatgtttgatgcttatgtcgacaccttttcagcaacttttagtgttcctatggaaaaacttaaggcacttgttgctacag |
| | | ctcacagcgagttagcaaagggtgtagattagatggtgtcctttctacattcgtgtcagctgcccgacaaggtgttgttgataccgatgttgacacaaa |
| | | ggatgttattgaatgtctcaaactttcacatcactctgacttagaagtgacaggtgacagttgtaacaatttcatgctcacctataataaggttgaaaa |
| | | catgacgcccagagatcttggcgcatgtattgactgtaatgcaaggcatatcaatgcccaagtagcaaaaagtcacaatgtttcactcatctggaatgt |
| | | aaaagactacatgtctttatctgaacagctgcgtaaacaaattcgtagtgctgccaagaagaacaacataccttttagactaacttgtgctacaactag |
| | | acaggttgtcaatgtcataactactaaaatctcactcaagggtggtaagattgttagtacttgttttaaacttatgcttaaggccacattattgtgcgt |
| | | tcttgctgcattggtttgttatatcgttatgccagtacatacattgtcaatccatgatggttacacaaatgaaatcattggttacaaagccattcagga |
| | | tggtgtcactcgtgacatcatttctactgatgattgttttgcaaataaacatgctggttttgacgcatggtttagccagcgtggtggttcatacaaaaa |
| | | tgacaaaagctgccctgtagtagctgctatcattacaagagagattggtttcatagtgcctggcttaccgggtactgtgctgagagcaatcaatggtga |
| | | cttcttgcattttctacctcgtgtttttagtgctgttggcaacatttgctacacaccttccaaactcattgagtatagtgattttgctacctctgcttg |
| | | cgttcttgctgctgagtgtacaatttttaaggatgctatgggcaaacctgtgccatattgttatgacactaatttgctagagggttctatttcttatag |
| | | tgagcttcgtccagacactcgttatgtgcttatggatggttccatcatacagtttcctaacacttacctggagggttctgttagagtagtaacaacttt |
| | | tgatgctgagtactgtagacatggtacatgcgaaaggtcagaagtaggtatttgcctatctaccagtggtagatgggttcttaataatgagcattacag |
| | | agctctatcaggagttttctgtggtgttgatgcgatgaatctcatagctaacatctttactcctcttgtgcaacctgtgggtgattagatgtgtctgct |
| | | tcagtagtggctggtggtattattgccatattggtgacttgtgctgcctactactttatgaaattcagacgtgtttttggtgagtacaaccatgttgtt |
| | | gctgctaatgcacttttgtttttgatgtctttcactatactctgtctggtaccagcttacagctttctgccgggagtctactcagtcttttacttgtac |
| | | ttgacattctatttcaccaatgatgtttcattcttggctcaccttcaatggtttgccatgttttctcctattgtgcctttttggataacagcaatctat |
| | | gtattctgtatttctctgaagcactgccattggttattaacaactatcttaggaaaagagtcatgtttaatggagttacatttagtaccttcgaggagg |
| | | ctgattgtgtacctttttgctcaacaaggaaatgtacctaaaattgcgtagcgagacactgttgccacttacacagtataacaggtatcttgctctata |
| | | taacaagtacaagtatttcagtggagccttagatactaccagctatcgtgaagcagcttgctgccacttagcaaaggctctaaatgactttagcaactc |
| | | aggtgctgatgttctctaccaaccaccacagacatcaatcacttctgctgttctgcagagtggttttaggaaaatggcattcccgtcaggcaaagttga |
| | | agggtgcatggtacaagtaacctgtggaactacaactcttaatggattgtggttggatgacacagtatactgtccaagacatgtcatttgcacagcaga |
| | | agacatgcttaatcctaactatgaagatctgctcattcgcaaatccaaccatagctttcttgttcaggctggcaatgttcaacttcgtgttattggcca |
| | | ttctatgcaaaattgtctgcttaggcttaaagttgatacttctaaccctaagacacccaagtataaatttgtccgtatccaacctggtcaaacattttc |
| | | agttctagcatgctacaatggttcaccatctggtgtttatcagtgtgccatgagacctaatcataccattaaaggttattccttaatggatcatgtggt |
| | | agtgttggttttaacattgattatgattgcgtgtattctgctatatgcatcatatggagcttccaacaggagtacacgctggtactgacttagaaggta |
| | | aattctatggtccatttgttgacagacaaactgcacaggctgcaggtacagacacaaccataacattaaatgttttggcatggctgtatgctgctgtta |
| | | tcaatggtgataggtggtttcttaatagattcaccactactttgaatgactttaaccttgtggcaatgaagtacaactatgaacctttgacacaagatc |
| | | atgttgacatattgggacctctttctgctcaaacaggaattgccgtcttagatatgtgtgctgctttgaaagagctgctgcagaatggtatgaatggtc |
| | | gtactatccttggtagcactattttagaagatgagtttacaccatttgatgttgttagacaatgctctggtgttaccttccaaggtaagttcaagaaaa |
| | | ttgttaagggcactcatcattggatgcttttaactttcttgacatcactattgattcttgttcaaagtacacagtggtcactgtttttctttgtttacg |
| | | agaatgctttcttgccatttactcttggtattatggcaattgctgcatgtgctatgctgcttgttaagcataagcacgcattcttgtgcttgtttctgt |
| | | taccttctcttgcaacagttgcttactttaatatggtctacatgcctgctagctgggtgatgcgtatcatgacatggcttgaattggctgacactagct |
| | | tgtaggttataggcttaaggattgtgttatgtatgcttcagctttagt-tttgcttattctcatgacagctcgcactgtttatgatgatgctgctagac |
| | | gtgtttggacactgatgaatgtcattacacttgtttacaaagtctactatggtaatgattagatcaagctatttccatgtgggccttagttatttctgt |
| | | aacctctaactattctggtgtcgttacgactatcatgtttttagctagagctatagtgtttgtgtgtgttgagtattacccattgttatttattactgg |
| | | caacaccttacagtgtatcatgcttgtttattgtttcttaggctattgttgctgctgctactttggccttttctgtttactcaaccgttacttcaggct |
| | | tactcttggtgtttatgactacttggtactacacaagaatttaggtatatgaactcccaggggcttttgcctcctaagagtagtattgatgattcaagc |
| | | ttaacattaagttgttgggtattggaggtaaaccatgtatcaaggttgctactgtacagtctaaaatgtctgacgtaaagtgcacatctgtggtactgc |
| | | tctcggttcttcaacaacttagagtagagtcatcttctaaattgtgggcacaatgtgtacaactccacaatgatattcttcttgcaaaagacacaactg |
| | | aagctttcgagaagatggtttctcttttgtctgttttgctatccatgcagggtgctgtagacattaataggttgtgcgaggaaatgctcgataaccgtg |
| | | ctactcttcaggctattgcttcagaatttagttattaccatcatatgccgcttatgccactgcccaggaggcctatgagcaggctgtagctaatggtga |
| | | ttctgaagtcgttctcaaaaagttaaagaaatctttgaatgtggctaaatctgagtttgaccgtgatgctgccatgcaacgcaagttggaaaagatggc |
| | | agatcaggctatgacccaaatgtacaaacaggcaagatctgaggacaagagggcaaaagtaactagtgctatgcaaacaatgctcttcactatgcttag |
| | | gaagcttgataatgatgcacttaacaacattatcaacaatgcgcgtgatggttgtgttccactcaacatcataccattgactacagcagccaaactcat |
| | | ggttgttgtccctgattatggtacctacaagaacacttgtgatggtaacacctttacatatgcatctgcactagggaaatccagcaagttgttgatgcg |
| | | gatagcaagattgttcaacttagtgaaattaacatggacaattcaccaaatttggcttggcctcttattgttacagctctaagagccaactcagctgtt |
| | | aaactacagaataatgaactgagtccagtagcactacgacagatgtcctgtgcggctggtaccacacaaacagcttgtactgatgacaatgcacttgcc |
| | | tactataacaattcgaagggaggtaggtttgtgctggcattactatcagaccaccaagatctcaaatgggctagattccctaagagtgatggtacaggt |
| | | acaatttacacagaactggaaccaccttgtaggtttgttacagacacaccaaaagggcctaaagtgaaatacttgtacttcatcaaaggcttaaacaac |
| | | ctaaatagaggtatggtgctgggcagtttagctgctacagtacgtcttcaggctggaaatgctacagaagtacctgccaattcaactgtgattccttct |
| | | gtgcttttgcagtagaccctgctaaagcatataaggattacctagcaagtggaggacaaccaatcaccaactgtgtgaagatgttgtgtacacacactg |
| | | gtacaggacaggcaattactgtaacaccagaagctaacatggaccaagagtcctttggtggtgcttcatgttgtagtattgtagatgccacattgacca |
| | | tccaaatcctaaaggattctgtgacttgaaaggtaagtacgtccaaatacctaccacttgtgctaatgacccagtgggttttacacttagaaacacagt |
| | | ctgtaccgtctgcggaatgtggaaaggttatggctgtagttgtgaccaactccgcgaacccttgatgcagtctgcggatgcatcaacgtttttaaacgg |
| | | gtttgcggtgtaagtgcagcccgtcttacaccgtgcggcacaggcactagtactgatgtcgtctacagggcttttgatatttacaacgaaaaagttgct |
| | | ggttttgcaaagttcctaaaaactaattgctgtcgcttccaggagaaggatgaggaaggcaatttattagactcttactttgtagttaagaggcatact |
| | | atgtctaactaccaacatgaagagactatttataacttggttaaagattgtccagcggttgctgtccatgactttttcaagtttagagtagatggtgac |
| | | atggtaccacatatatcacgtcagcgtctaactaaatacacaatggctgatttagtctatgactacgtcattttgatgagggtaattgtgatacattaa |
| | | aagaaatactcgtcacatacaattgctgtgatgatgattatttcaataagaaggattggtatgacttcgtagagaatcctgacatcttacgcgtatatg |
| | | ctaacttaggtgagcgtgtacgccaatcattattaaagactgtacaattctgcgatgctatgcgtgatgcaggcattgtaggcgtactgacattagata |
| | | atcaggatcttaatgggaactggtacgatttcggtgatttcgtacaagtagcaccaggctgcggagttcctattgtggattcatattactcattgctga |
| | | tgcccatcctcactttgactagggcattggctgctgagtcccatatggatgctgatctcgcaaaaccacttattaagtgggatttgctgaaatatgatt |
| | | ttacggaagagagactttgtctcttcgaccgttattttaaatattgggaccagacataccatcccaattgtattaactgtttggatgataggtgtatcc |
| | | ttcattgtgcaaactttaatgtgttattttctactgtgtttccacctacaagttttggaccactagtaagaaaaatatttgtagatggtgttccttttg |
| | | ttgtttcaactggataccattttcgtgagttaggagtcgtacataatcaggatgtaaacttacatagctcgcgtctcagtttcaaggaacttttagtgt |
| | | atgctgctgatccagctatgcatgcagcttctggcaatttattgctagataaacgcactacatgcttttcagtagctgcactaacaaacaatgttgatt |
| | | tcaaactgtcaaacccggtaattttaataaagacttttatgactttgctgtgtctaaaggtttctttaaggaaggaagttctgttgaactaaaacactt |
| | | cttctttgctcaggatggcaacgctgctatcagtgattatgactattatcgttataatctgccaacaatgtgtgatatcagacaactcctattcgtagt |
| | | tgaagttgttgataaatactttgattgttacgatggtggctgtattaatgccaaccaagtaatcgttaacaatctggataaatcagctggtttcccatt |
| | | taataaatggggtaaggctagactttattatgactcaatgagttatgaggatcaagatgcacttttcgcgtatactaagcgtaatgtcatccctactat |
| | | aactcaaatgaatcttaagtatgccattagtgcaaagaatagagctcgcaccgtagaggtgtactatctgtagtactatgacaaatagacagtttcatc |
| | | agaaattattgaagtcaatagccgccactagaggagctactgtggtaattggaacaagcaagttttacggtggctggcataatatgttaaaaactgttt |
| | | acagtgatgtagaaactccacaccttatgggttgggattatccaaaatgtgacagagccatgcctaacatgcttaggataatggcctctcttgttcttg |
| | | ctcgcaaacataacacttgctgtaacttatcacaccgtttctacaggttagctaacgagtgtgcgcaagtattaagtgagatggtcatgtgtggcggct |
| | | cactatatgttaaaccaggtggaacatcatccggtgatgctacaactgcttatgctaatagtgtctttaacatttgtcaagctgttacagccaatgtaa |
| | | atgcacttattcaactgatggtaataagatagctgacaagtatgtccgcaatctacaacacaggctctatgagtgtactatagaaatagggatgttgat |
| | | catgaattcgtggatgagttttacgcttacctgcgtaaacatttctccatgatgattctttctgatgatgccgttgtgtgctataacagtaactatgcg |
| | | gctcaaggtttagtagctagcattaagaactttaaggcagttattattatcaaaataatgtgttcatgtctgaggcaaaatgttggactgagactgacc |
| | | ttactaaaggacctcacgaattttgctcacagcatacaatgctagttaaacaaggagatgattacgtgtacctgccttacccagatccatcaagaatat |
| | | taggcgcaggctgttttgtcgatgatattgtcaaaacagatggtacacttatgattgaaaggttcgtgtcactggctattgatgcttacccacttacaa |
| | | aacatcctaatcaggagtatgctgatgtctttcacttgtatttacaatacattagaaagttacatgatgagcttactggccacatgttggacatgtatt |
| | | ccgtaatgctaactaatgataacacctcacggtactgggaacctgagttttatgaggctatgtacacaccacatacagtcttgcaggctgtaggtgctt |
| | | gtgtattgtgcaattcacagacttcacttcgttgcggtgcctgtattaggagaccattcctatgttgcaagtgctgctatgaccatgtcatttcaacat |
| | | cacacaaattagtgttgtctgttaatccctatgtttgcaatgccccaggttgtgatgtcactgatgtgacacaactgtatctaggaggtatgagctatt |
| | | attgcaagtcacataagcctcccattagttttccattatgtgctaatggtcaggtttttggtttatacaaaaacacatgtgtaggcagtgacaatgtca |
| | | ctgacttcaatgcgatagcaacatgtgattggactaatgctggcgattacatacttgccaacacttgtactgagagactcaagcttttcgcagcagaaa |
| | | cgctcaaagccactgaggaaacatttaagagtcatatggtattgccactgtacgcgaagtactctctgacagagaattgcatctttcatgggaggttgg |
| | | aaaacctagaccaccattgaacagaaactatgtattactggttaccgtgtaactaaaaatagtaaagtacagattggagagtacacctttgaaaaaggt |
| | | gactatggtgatgctgttgtgtacagaggtactacgacatacaagttgaatgttggtgattactttgtgttgacatctcacactgtaatgccacttagt |
| | | gcacctactctagtgccacaagagcactatgtgagaattactggcttgtacccaacactcaacatctcagatgagttttctagcaatgttgcaaattat |
| | | caaaaggtcggcatgcaaaagtactctacactccaaggaccacctggtactggtaagagtcattttgccatcggacttgactctattacccatctgctc |
| | | gcatagtgtatacggcatgactcatgcagctgttgatgccctatgtgaaaaggcattaaaatatttgcccatagataaatgtagtagaatcatacctgc |
| | | gcgtgcgcgcgtagagtgttttgataaattcaaagtgaattcaacactagaacagtatgttttctgcactgtaaatgcattgccagaaacaactgctga |
| | | cattgtagtctttgatgaaatctctatggctactaattatgacttgagtgttgtcaatgctagacttcgtgcaaaacactacgtctatattggcgatcc |
| | | tgctcaattaccagccccccgcacattgctgactaaaggcacactagaaccagaatattttaattcagtgtgcagacttatgaaaacaataggtccaga |
| | | catgttccttggaacttgtcgccgttgtcctgctgaaattgttgacactgtgagtgctttagtttatgacaataagctaaaagcacacaaggataagtc |
| | | agctcaatgcttcaaaatgttctacaaaggtgttattacacatgatgtttcatctgcaatcaacagacctcaaataggcgttgtaagagaatttcttac |
| | | acgcaatcctgcttggagaaaagctgtttttatctcaccttataattcacagaacgctgtagcttcaaaaatcttaggattgcctacgcagactgttga |
| | | ttcatcacagggttctgaatatgactatgtcatattcacacaaactactgaaacagcacactcttgtaatgtcaaccgcttcaatgtggctatcacaag |
| | | ggcaaaaattggcattttgtgcataatgtctgatagagatctttatgacaaactgcaatttacaagtctagaaataccacgtcgcaatgtggctacatt |
| | | acaagcagaaaatgtaactggactttttaaggactgtagtaagatcattactggtcttcatcctacacaggcacctacacacctcagcgttgatataaa |
| | | gttcaagactgaaggattatgtgttgacataccaggcataccaaaggacatgacctaccgtagactcatctctatgatgggtttcaaaatgaattacca |
| | | agtcaatggttaccctaatatgtttatcacccgcgaagaagctattcgtcacgttcgtgcgtggattggctttgatgtagagggctgtcatgcaactag |
| | | agatgctgtgggtactaacctacctctccagctaggattttctacaggtgttaacttagtagctgtaccgactggttatgttgacactgaaaataacac |
| | | agaattcaccagagttaatgcaaaacctccaccaggtgaccagtttaaacatcttataccactcatgtataaaggcttgccctggaatgtagtgcgtat |
| | | taagatagtacaaatgctcagtgatacactgaaaggattgtcagacagagtcgtgttcgtcctttgggcgcatggctttgagcttacatcaatgaagta |
| | | ctttgtcaagattggacctgaaagaacgtgttgtctgtgtgacaaacgtgcaacttgatttctacttcatcagatacttatgcctgctggaatcattct |
| | | gtgggttttgactatgtctataacccatttatgattgatgttcagcagtggggctttacgggtaaccttcagagtaaccatgaccaacattgccaggta |
| | | catggaaatgcacatgtggctagttgtgatgctatcatgactagatgtttagcagtccatgagtgattgttaagcgcgttgattggtctgttgaatacc |
| | | ctattataggagatgaactgagggttaattctgcttgcagaaaagtacaacacatggttgtgaagtctgcattgcttgctgataagtttccagttcttc |
| | | atgacattggaaatccaaaggctatcaagtgtgtgcctcaggctgaagtagaatggaagttctacgatgctcagccatgtagtgacaaagcttacaaaa |
| | | tagaggaactcttctattcttatgctacacatcacgataaattcactgatggtgtttgtttgttttggaattgtaacgttgatcgttacccagccaatg |
| | | caattgtgtgtaggtttgacacaagagtcttgtcaaacttgaacttaccaggctgtgatggtggtagtttgtatgtgaataagcatgcattccacactc |
| | | cagctttcgataaaagtgcatttactaatttaaagcaattgcctttcttttactattctgatagtccttgtgagtctcatggcaaacaagtagtgtcgg |
| | | atattgattatgttccactcaaatctgctacgtgtattacacgatgcaatttaggtggtgctgtttgcagacaccatgcaaatgagtaccgacagtact |
| | | tggatgcatataatatgatgatttctgctggatttagcctatggatttacaaacaatttgatacttataacctgtggaatacatttaccaggttacaga |
| | | gtttagaaaatgtggcttataatgttgttaataaaggacactttgatggacacgccggcgaagcacctgtttccatcattaataatgctgtttacacaa |
| | | aggtagatggtattgatgtggagatctttgaaaataagacaacacttcctgttaatgttgcatttgagctttgggctaagcgtaacattaaaccagtgc |
| | | cagagattaagatactcaataatttgggtgttgatatcgctgctaatactgtaatctgggactacaaaagagaagccccagcacatgtatctacaatag |
| | | gtgtctgcacaatgactgacattgccaagaaacctactgagagtgcttgttcttcacttactgtcttgtttgatggtagagtggaaggacaggtagacc |
| | | tttttagaaacgcccgtaatggtgttttaataacagaaggttcagtcaaaggtctaacaccttcaaagggaccagcacaagctagcgtcaatggagtca |
| | | cattaattggagaatcagtaaaaacacagtttaactactttaagaaagtagacggcattattcaacagttgcctgaaacctactttactcagagcagag |
| | | acttagaggattttaagcccagatcacaaatggaaactgactttctcgagctcgctatggatgaattcatacagcgatataagctcgagggctatgcct |
| | | tcgaacacatcgtttatggagatttcagtcatggacaacttggcggtcttcatttaatgataggcttagccaagcgctcacaagattcaccacttaaat |
| | | tagaggattttatccctatggacagcacagtgaaaaattacttcataacagatgcgcaaacaggttcatcaaaatgtgtgtgttctgtgattgatcttt |
| | | tacttgatgactttgtcgagataataaagtcacaagatttgtcagtgatttcaaaagtggtcaaggttacaattgactatgctgaaatttcattcatgc |
| | | tttggtgtaaggatggacatgttgaaaccttctacccaaaactacaagcaagtcaagcgtggcaaccaggtgttgcgatgcctaacttgtacaagatgc |
| | | aaagaatgcttcttgaaaagtgtgaccttcagaattatggtgaaaatgctgttataccaaaaggaataatgatgaatgtcgcaaagtatactcaactgt |
| | | gtcaatacttaaatacacttactttagctgtaccctacaacatgagagttattcactttggtgctggctctgataaaggagttgcaccaggtacagctg |
| | | tgctcagacaatggttgccaactggcacactacttgtcgattcagatcttaatgacttcgtaccgacgcagattctactttaattggagactgtgcaac |
| | | agtacatacggctaataaatgggaccttattattagcgatatgtatgaccctaggaccaaacatgtgacaaaagagaatgactctaaagaagggttttt |
| | | cacttatctgtgtggatttataaagcaaaaactagccctgggtggttctatagctgtaaagataacagagcattcttggaatgctgacctttacaagct |
| | | tatgggccatttctcatggtggacagcttttgttacaaatgtaaatgcatcatcatcggaagcatttttaattggggctaactatcttggcaagccgaa |
| | | ggaacaaattgatggctataccatgcatgctaactacattttctggaggaacacaaatcctatccagttgtcttcctattcactctttgacatgagcaa |
| | | atttcctcttaaattaagaggaactgctgtaatgtctcttaaggagaatcaaatcaatgatatgatttattctcttctggaaaaaggtaggcttatcat |
| | | tagagaaaacaacagagttgtggtttcaagtgatattcttgttaacaactaaacgaacatgtttattttcttattatttcttactctcactagtggtag |
| | | tgaccttgaccggtgcaccacttttgatgatgttcaagctcctaattacactcaacatacttcatctatgaggggggtttactatcctgatgaaatttt |
| | | tagatcagacactattatttaactcaggatttatttcttccattttattctaatgttacagggtttcatactattaatcatacgtttggcaaccctgtc |
| | | ataccttttaaggatggtatttattttgctgccacagagaaatcaaatgttgtccgtggttgggtttttggttctaccatgaacaacaagtcacagtcg |
| | | gtgattattattaacaattctactaatgttgttatacgagcatgtaactttgaattgtgtgacaaccattattgctgtttctaaacccatgggtacaca |
| | | gacacatactatgatattcgataatgcatttaattgcactttcgagtacatatctgatgccttttcgcttgatgtttcagaaaagtcaggtaattttaa |
| | | acacttacgagagtttgtgtttaaaaataaagatgggtttctctatgtttataagggctatcaacctatagatgtagttcgtgatctaccttctggttt |
| | | taacactttgaaacctatttttaagttgcctcttggtattaacattacaaattttagagccattcttacagccttttcacctgctcaagacatttgggg |
| | | cacgtcagctgcagcctattttgttggctatttaaagccaactacatttatgctcaagtatgatgaaaatggtacaatcacagatgctgttgattgttc |
| | | tcaaaatccacttgctgaactcaaatgctctgttaagagattgagattgacaaaggaatttaccagacctctaatttcagggttgttccctcaggagat |
| | | gttgtgagattccctaatattacaaacttgtgtccttttggagaggtttttaatgctactaaattcccttctgtctatgcatgggagagaaaaaaaatt |
| | | tctaattgtgttgctgattactctgtgctctacaactcaacatttttttcaacctttaagtgctatggcgtttctgccactaagttgaatgatctttgc |
| | | ttctccaatgtctatgcagattcttttgtagtcaagggagatgatgtaagacaaatagcgccaggacaaactggtgttattgctgattataattataaa |
| | | ttgccagatgatttcatgggttgtgtccttgcttggaatactaggaacattgatgctacttcaactggtaattataattataaatataggtatcttaga |
| | | catggcaagcttaggccctttgagagagacatatctaatgtgcctttctcccctgatggcaaaccttgcaccccacctgctcttaattgttattggcca |
| | | ttaaatgattatggtttttacaccactactggcattggctaccaaccttacagagttgtagtactttcttttgaacttttaaatgcaccggccacggtt |
| | | tgtggaccaaaattatccactgaccttattaagaaccagtgtgtcaattttaattttaatggactcactggtactggtgtgttaactccttcttcaaag |
| | | agatttcaaccatttcaacaatttggccgtgatgtttctgatttcactgattccgttcgagatcctaaaacatctgaaatattagacatttcaccttgc |
| | | gcttttgggggtgtaagtgtaattacacctggaacaaatgcttcatctgaagttgctgttctatatcaagatgttaactgcactgatgtttctacagca |
| | | attcatgcagatcaactcacaccagcttggcgcatatattctactggaaacaatgtattccagactcaagcaggctgtcttataggagctgagcatgtc |
| | | gacacttcttatgagtgcgacattcctattggagctggcatttgtgctagttaccatacagtttctttattacgtagtactagccaaaaatctattgtg |
| | | gcttatactatgtctttaggtgctgatagttcaattgcttactctaataacaccattgctatacctactaacttttcaattagcattactacagaagta |
| | | atgcctgtttctatggctaaaacctccgtagattgtaatatgtacatctgcggagattctactgaatgtgctaatttgcttctccaatatggtagattt |
| | | gcacacaactaaatcgtgcactctcaggtattgctgctgaacaggatcgcaacacacgtgaagtgttcgctcaagtcaaacaaatgtacaaaaccccaa |
| | | ctttgaaatattttggtggttttaatttttcacaaatattacctgaccctctaaagccaactaagaggtcttttattgaggacttgctctttaataagg |
| | | tgacactcgctgatgctggcttcatgaagcaatatggcgaatgcctaggtgatattaatgctagagatctcatttgtgcgcagaagttcaatggactta |
| | | cagtgttgccacctctgctcactgatgatatgattgctgcctacactgctgctctagttagtggtactgccactgctggatggacatttggtgctggcg |
| | | ctgctcttcaaataccttttgctatgcaaatggcatataggttcaatggcattggagttacccaaaatgttctctatgagaaccaaaaacaaatcgcca |
| | | accaatttaacaaggcgattagtcaaattcaagaatcacttacaacaacatcaactgcattgggcaagctgcaagacgttgttaaccagaatgctcaag |
| | | cattaaacacacttgttaaacaacttagctctaattttggtgcaatttcaagtgtgctaaatgatatcctttcgcgacttgataaagtcgaggcggagg |
| | | tacaaattgacaggttaattacaggcagacttcaaagccttcaaacctatgtaacacaacaactaatcagggctgctgaaatcagggcttctgctaatc |
| | | ttgctgctactaaaatgtctgagtgtgttcttggacaatcaaaaagagttgacttttgtggaaagggctaccaccttatgtccttcccacaagcagccc |
| | | cgcatggtgttgtcttcctacatgtcacgtatgtgccatcccaggagaggaacttcaccacagcgccagcaatttgtcatgaaggcaaagcatacttcc |
| | | ctcgtgaaggtgtttttgtgtttaatggcacttcttggtttattacacagaggaacttcttttctccacaaataattactacagacaatacatttgtct |
| | | caggaaattgtgatgtcgttattggcatcattaacaacacagtttatgatcctctgcaacctgagcttgactcattcaaagaagagctggacaagtact |
| | | tcaaaaatcatacatcaccagatgttgatcttggcgacatttcaggcattaacgcttctgtcgtcaacattcaaaaagaaattgaccgcctcaatgagg |
| | | tcgctaaaaatttaaatgaatcactcattgaccttcaagaattgggaaaatatgagcaatatattaaatggccttggtatgtttggctcggcttcattg |
| | | ctggactaattgccatcgtcatggttacaatcttgattgttgcatgactagttgttgcagttgcctcaagggtgcatgctcttgtggttcttgctgcaa |
| | | gtttgatgaggatgactctgagccagttctcaagggtgtcaaattacattacacataaacgaacttatggatttgtttatgagattttttactcttaga |
| | | tcaattactgcacagccagtaaaaattgacaatgcttctcctgcaagtactgttcatgctacagcaacgataccgctacaagcctcactccattcggat |
| | | ggcttgttattggcgttgcatttcttgctgtttttcagagcgctaccaaaataattgcgctcaataaaagatggcagctagccattataagggcttcca |
| | | gttcatttgcaatttactgctgctatttgttaccatctattcacatcttttgcttgtcgctgcaggtatggaggcgcaatttttgtacctctatgcctt |
| | | gatatattttctacaatgcatcaacgcatgtagaattattatgagatgttggctttgttggaagtgcaaatccaagaacccattactttatgatgccaa |
| | | ctactttgtttgctggcacacacataactatgactactgtataccatataacagtgtcacagatacaattgtcgttactgaaggtgacggcatttcaac |
| | | accaaaactcaaagaagactaccaaattggtggttattctgaggataggcactcaggtgttaaagactatgtcgttgtacatggctatttcaccgaagt |
| | | ttactaccagcttgagtctacacaaattactacagacactggtattgaaaatgctacattcttcatctttaacaagcttgttaaagacccaccgaatgt |
| | | gcaaatacacacaatcgacggctcttcaggagttgctaatccagcaatggatccaatttatgatgagccgacgacgactactagcgtgcctttgtaagc |
| | | acaagaaagtgagtacgaacttatgtactcattcgtttcggaagaaacaggtacgttaatagttaatagcgtacttctttttcttgattcgtggtattc |
| | | ttgctagtcacactagccatccttactgcgcttcgattgtgtgcgtactgctgcaatattgttaacgtgagtttagtaaaaccaacggtttacgtctac |
| | | tcgcgtgttaaaaatctgaactcttctgaaggagttcctgatcttctggtctaaacgaactaactattattattattctgtttggaactttaacattgc |
| | | ttatcatggcagacaacggtactattaccgttgaggagcttaaacaactcctggaacaatggaacctagtaataggtttcctattcctagcctggatta |
| | | tgttactacaatttgcctattctaatcggaacaggtttttgtacataataaagcttgttttcctaggctcttgtggccagtaacacttgcttgttttgt |
| | | gcttgctgctgtctacagaattaattgggtgactggcgggattgcgattgcaatggcttgtattgtaggcttgatgtggcttagctacttcgttgcttc |
| | | cttcaggctgtttgctcgtacccgctcaatgtggtcattcaacccagaaacaaacattcttctcaatgtgcctctccgggggacaattgtgaccagacc |
| | | gctcatggaaagtgaacttgtcattggtgctgtgatcattcgtggtcacttgcgaatggccggacactccctagggcgctgtgacattaaggacctgcc |
| | | aaaagagatcactgtggctacatcacgaacgattcttattacaaattaggagcgtcgcagcgtgtaggcactgattcaggttttgctgcatacaaccgc |
| | | taccgtattggaaactataaattaaatacagaccacgccggtagcaacgacaatattgattgctagtacagtaagtgacaacagatgtttcatcttgtt |
| | | gacttccaggttacaatagcagagatattgattatcattatgaggactttcaggattgctatttggaatcttgacgttataataagttcaatagtgaga |
| | | caattatttaagcctctaactaagaagaattattcggagttagatgatgaagaacctatggagttagattatccataaaacgaacatgaaaattattct |
| | | cttcctgacattgattgtatttacatcttgcgagctatatcactatcaggagtgtgttagaggtacgactgtactactaaaagaaccttgcccatcagg |
| | | aacatacgagggcaattcaccatttcaccctcttgctgacaataaatttgcactaacttgcactagcacacactttgatttgcttgtgctgacggtact |
| | | cgacatacctatcagctgcgtgcaagatcagtttcaccaaaacttttcatcagacaagaggaggttcaacaagagctctactcgccactttttctcatt |
| | | gttgctgctctagtatttttaatactttgcttcaccattaagagaaagacagaatgaatgagctcactttaattgacttctatttgtgctttttagcct |
| | | ttctgctattccttgttttaataatgcttattatattttggttttcactcgaaatccaggatctagaagaaccttgtaccaaagtctaaacgaacatga |
| | | aacttctcattgttttgacttgtatttctctatgcagttgcatatgcactgtagtacagcgctgtgcatctaataaacctcatgtgcttgaagatcctt |
| | | gtaaggtacaacactaggggtaatacttatagcactgcttggctttgtgctctaggaaaggttttaccttttcatagatggcacactatggttcaaaca |
| | | tgcacacctaatgttactatcaactgtcaagatccagctggtggtgcgcttatagctaggtgttggtaccttcatgaaggtcaccaaactgctgcattt |
| | | agagacgtacttgttgttttaaataaacgaacaaattaaaatgtctgataatggaccccaatcaaaccaacgtagtgccccccgcattacatttggtgg |
| | | acccacagattcaactgacaataaccagaatggaggacgcaatggggcaaggccaaaacagcgccgaccccaaggtttacccaataatactgcgtatgg |
| | | ttcacagctctcactcagcatggcaaggaggaacttagattccctcgaggccagggcgttccaatcaacaccaatagtggtccagatgaccaaattggc |
| | | tactaccgaagagctacccgacgagttcgtggtggtgacggcaaaatgaaagagctcagccccagatggtacttctattacctaggaactggcccagaa |
| | | gcttcacttccctacggcgctaacaaagaaggcatcgtatgggttgcaactgagggagccttgaatacacccaaagaccacattggcacccgcaatcct |
| | | aataacaatgctgccaccgtgctacaacttcctcaaggaacaacattgccaaaaggcttctacgcagagggaagcagaggcggcagtcaagcctcttct |
| | | cgctcctcatcacgtagtcgcggtaattcaagaaattcaactcctggcagcagtaggggaaattctcctgctcgaatggctagcggaggtggtgaaact |
| | | gccctcgcgctattgctgctagacagattgaaccagcttgagagcaaagtttctggtaaaggccaacaacaacaaggccaaactgtcactaagaaatct |
| | | gctgctgaggcatctaaaaagcctcgccaaaaacgtactgccacaaaacagtacaacgtcactcaagcatttgggagacgtggtccagaacaaacccaa |
| | | ggaaatttcggggaccaagacctaatcagacaaggaactgattacaaacattggccgcaaattgcacaatttgctccaagtgcctctgcattctttgga |
| | | atgtcacgcattggcatggaagtcacaccttcgggaacatggctgacttatcatggagccattaaattggatgacaaagatccacaattcaaagacaac |
| | | gtcatactgctgaacaagcacattgacgcatacaaaacattcccaccaacagagcctaaaaaggacaaaaagaaaaagactgatgaagctcagcctttg |
| | | ccgcagagacaaaagaagcagcccactgtgactcttcttcctgcggctgacatggatgatttctccagacaacttcaaaattccatgagtggagcttct |
| | | gctgattcaactcaggcataaacactcatgatgaccacacaaggcagatgggctatgtaaacgttttcgcaattccgtttacgatacatagtctactct |
| | | tgtgcagaatgaattctcgtaactaaacagcacaagtaggtttagttaactttaatctcacatagcaatctttaatcaatgtgtaacattagggaggac |
| | | ttgaaagagccaccacattttcatcgaggccacgcggagtacgatcgagggtacagtgaataatgctagggagagctgcctatatggaagagccctaat |
| | | gtgtaaaattaattttagtagtgctatccccatgtgattttaatagcttcttaggagaatgacaaaaaaaaaaaaaaaaaaaaaaaa |
| 5537 | MERS-CoV genome | gatttaagtgaatagettggctatctcacttcccctcgttctcttgcagaactttgattttaacgaacttaaataaaagccctgttgtttagcgtatcg |
| | Genbank Accession No. NC_019843.3 | ttgcacttgtctggtgggattgtggcattaatttgcctgctcatctaggcagtggacatatgctcaacactgggtataattctaattgaatactatttt |
| | | tcagttagagcgtcgtgtctcttgtacgtctcggtcacaatacacggtttcgtccggtgcgtggcaattcggggcacatcatgtctttcgtggctggtg |
| | | tgaccgcgcaaggtgcgcgcggtacgtatcgagcagcgctcaactctgaaaaacatcaagaccatgtgtctctaactgtgccactctgtggttcaggaa |
| | | acctggttgaaaaactttcaccatggttcatggatggcgaaaatgcctatgaagtggtgaaggccatgttacttaaaaaggagccacttctctatgtgc |
| | | ccatccggctggctggacacactagacacctcccaggtcctcgtgtgtacctggttgagaggctcattgcttgtgaaaatccattcatggttaaccaat |
| | | tggcttatagctctagtgcaaatggcagcctggttggcacaactttgcagggcaagcctattggtatgttcttcccttatgacatcgaacttgtcacag |
| | | gaaagcaaaatattctcctgcgcaagtatggccgtggtggttatcactacaccccattccactatgagcgagacaacacctcttgccctgagtggatgg |
| | | acgattttgaggcggatcctaaaggcaaatatgcccagaatctgcttaagaagttgattggcggtgatgtcactccagttgaccaatacatgtgtggcg |
| | | ttgatggaaaacccattagtgcctacgcatttttaatggccaaggatggaataaccaaactggctgatgttgaagcggacgtcgcagcacgtgctgatg |
| | | acgaaggcttcatcacattaaagaacaatctatatagattggtttggcatgttgagcgtaaagacgttccatatcctaagcaatctatttttactatta |
| | | atagtgtggtccaaaaggatggtgttgaaaacactcctcctcactattttactettggatgcaaaattttaacgctcaccccacgcaacaagtggagtg |
| | | gcgtttctgacttgtecctcaaacaaaaactcctttacaccttctatggtaaggagtcacttgagaacccaacctacatttaccactccgcattcattg |
| | | agtgtggaagttgtggtaatgattcctggcttacagggaatgctatccaagggtttgcctgtggatgtggggcatcatatacagctaatgatgtcgaag |
| | | tccaatcatctggcatgattaagccaaatgctcttctttgtgctacttgcccctttgctaagggtgatagctgttcttctaattgcaaacattcagttg |
| | | ctcagttggttagttacctttctgaacgctgtaatgttattgctgattctaagtecttcacacttatattggtggcgtagcttacgcctactttggatg |
| | | tgaggaaggtactatgtactttgtgcctagagctaagtctgttgtctcaaggattggagactccatctttacaggctgtactggctcttggaacaaggt |
| | | cactcaaattgctaacatgttatggaacagactcagcattcccttaactttgtgggagagttcgttgtcaacgatgttgtcctcgcaattctctctgga |
| | | accacaactaatgttgacaaaatacgccagcttctcaaaggtgtcacccttgacaagttgcgtgattatttagctgactatgacgtagcagtcactgcc |
| | | ggcccattcatggataatgctattaatgttggtggtacaggattacagtatgccgccattactgcaccttatgtagttctcactggcttaggtgagtcc |
| | | tttaagaaagttgcaaccataccgtataaggtttgcaactctgttaaggatactctggcttattatgctcacagcgtgttgtacagagtttttccttat |
| | | gacatggattctggtgtgtcatcctttagtgaactactttttgattgcgttgatctttcagtagcttctacctattttttagtccgcatcttgcaagat |
| | | aagactggcgactttatgtctacaattattacttcctgccaaactgctgttagtaagcttctagatacatgttttgaagctacagaagcaacatttaac |
| | | ttcttgttagatttggcaggattgttcagaatctttctccgcaatgcctatgtgtacacttcacaagggtttgtggtggtcaatggcaaagtttctaca |
| | | cttgtcaaacaagtgttagacttgcttaataagggtatgcaacttttgcatacaaaggtctcctgggctggttctaaaatcattgctgttatctacagc |
| | | ggcagggagtctctaatattcccatcgggaacctattactgtgtcaccactaaggctaagtccgttcaacaagatcttgacgttattttgcctggtgag |
| | | ttttccaagaagcagttaggactgctccaacctactgacaattctacaactgttagtgttactgtatccagtaacatggttgaaactgttgtgggtcaa |
| | | cttgagcaaactaatatgcatagtcctgatgttatagtaggtgactatgtcattattagtgaaaaattgtttgtgcgtagtaaggaagaagacggattt |
| | | gccttctaccctgcttgcactaatggtcatgctgtaccgactctctttagacttaagggaggtgcacctgtaaaaaaagtagcctttggcggtgatcaa |
| | | gtacatgaggttgctgctgtaagaagtgttactgtcgagtacaacattcatgctgtattagacacactacttgcttcttctagtcttagaacctttgtt |
| | | gtagataagtctttgtcaattgaggagtttgctgacgtagtaaaggaacaagtctcagacttgcttgttaaattactgcgtggaatgccgattccagat |
| | | tttgatttagacgattttattgacgcaccatgctattgctttaacgctgagggtgatgcatcctggtcttctactatgatcttctctcttcaccccgtc |
| | | gagtgtgacgaggagtgttctgaagtagaggcttcagatttagaagaaggtgaatcagagtgcatttctgagacttcaactgaacaagttgacgtttct |
| | | catgagacttctgacgacgagtgggctgctgcagttgatgaagcgttccctctcgatgaagcagaagatgttactgaatctgtgcaagaagaagcacaa |
| | | ccagtagaagtacctgttgaagatattgcgcaggttgtcatagctgacaccttacaggaaactcctgttgtgcctgatactgttgaagtcccaccgcaa |
| | | gtggtgaaacttccgtctgcacctcagactatccagcccgaggtaaaagaagttgcacctgtctatgaggctgataccgaacagacacagaatgttact |
| | | gttaaacctaagaggttacgcaaaaagcgtaatgttgaccattgtccaattttgaacataaggttattacagagtgcgttaccatagttttaggtgacg |
| | | caattcaagtagccaagtgctatggggagtctgtgttagttaatgctgctaacacacatcttaagcatggcggtggtatcgctggtgctattaatgcgg |
| | | cttcaaaaggggctgtccaaaaagagtcagatgagtatattctggctaaagggccgttacaagtaggagattcagttctcttgcaaggccattctctag |
| | | ctaagaatatcctgcatgtcgtaggcccagatgcccgcgctaaacaggatgtttctctccttagtaagtgctataaggctatgaatgcatatcctcttg |
| | | tagtcactcctcttgtttcagcaggcatatttggtgtaaaaccagagtgtatttgattatcttattagggaggctaagactagagttttagtcgtcgtt |
| | | aattcccaagatgtctataagagtcttaccatagttgacattccacagagtttgactttttcatatgatgggttacgtggcgcaatacgtaaagctaaa |
| | | gattatggttttactgtttttgtgtgcacagacaactctgctaacactaaagttcttaggaacaagggtgttgattatactaagaagtttcttacagtt |
| | | gacggtgtgcaatattattgctacacgtctaaggacactttagatgatatcttacaacaggctaataagtctgttggtattatatctatgcctttggga |
| | | tatgtgtctcatggtttagacttaatgcaagcagggagtgtcgtgcgtagagttaacgtgccctacgtgtgtctcctagctaataaagagcaagaagct |
| | | attttgatgtctgaagacgttaagttaaacccttcagaagattttataaagcacgtccgcactaatggtggttacaattcttggcatttagtcgagggt |
| | | gaactattggtgcaagacttacgcttaaataagctcctgcattggtctgatcaaaccatatgctacaaggatagtgtgttttatgttgtaaagaatagt |
| | | acagcttttccatttgaaacactttcagcatgtcgtgcgtatttggattcacgcacgacacagcagttaacaatcgaagtcttagtgactgtcgatggt |
| | | gtaaattttagaacagtcgttctaaataataagaacacttatagatcacagcttggatgcgttttctttaatggtgctgatatttctgacaccattcct |
| | | gatgagaaacagaatggtcacagtttatatctagcagacaatttgactgctgatgaaacaaaggcgcttaaagagttatatggccccgttgatcctact |
| | | ttcttacacagattctattcacttaaggctgcagtccatgggtggaagatggttgtgtgtgataaggtacgttctctcaaattgagtgataataattgt |
| | | tatcttaatgcagttattatgacacttgatttattgaaggacattaaatttgttatacctgactacagcatgcatttatgaaacataagggcggtgatt |
| | | caactgacttcatagccctcattatggcttatggcaattgcacatttggtgaccagatgatgcctctcggttacttcataccgtgcttgcaaaggctga |
| | | gttatgctgttctgcacgcatggtttggagagagtggtgcaatgtctgtggcataaaagatgttgttctacaaggcttaaaagcttgttgttacgtggg |
| | | tgtgcaaactgttgaagatctgcgtgctcgcatgacatatgtatgccagtgtggtggtgaacgtcatcggcaattagtcgaacacaccaccccctggtt |
| | | gctgctctcaggcacaccaaatgaaaaattggtgacaacctccacggcgcctgattttgtagcatttaatgtctttcagggcattgaaacggctgttgg |
| | | ccattatgttcatgctcgcctgaagggtggtcttattttaaagtttgactaggcaccgttagcaagacttcagactggaagtgcaaggtgacagatgta |
| | | cttttccccggccaaaaatacagtagcgattgtaatgtcgtacggtattctttggacggtaatttcagaacagaggttgatcccgacctatctgattct |
| | | atgttaaggatggtaaatactttacaagtgaaccacccgtaacatattcaccagctacaattttagctggtagtgtctacactaatagctgccttgtat |
| | | cgtctgatggacaacctggcggtgatgctattagtttgagttttaataaccttttagggtttgattctagtaaaccagtcactaagaaatacacttact |
| | | ccttcttgcctaaagaagacggcgatgtgttgttggctgagtttgacacttatgaccctatttataagaatggtgccatgtataaaggcaaaccaattc |
| | | tttgggtcaataaagcatcttatgatactaatcttaataagttcaatagagctagtttgcgtcaaatttttgacgtagcccccattgaactcgaaaata |
| | | aattcacacctttgagtgtggagtctacaccagttgaacctccaactgtagatgtggtagcacttcaacaggaaatgacaattgtcaaatgtaagggtt |
| | | taaataaacctttcgtgaaggacaatgtcagtttcgttgctgatgattcaggtactcccgttgttgagtatctgtctaaagaagacctacatacattgt |
| | | atgtagaccctaagtatcaagtcattgtcttaaaagacaatgtactttcttctatgcttagattgcacaccgttgagtcaggtgatattaacgttgttg |
| | | cagcttccggatctttgacacgtaaagtgaagttactatttagggcttcattttatttcaaagaatttgctacccgcactttcactgctaccactgctg |
| | | taggtagttgtataaagagtgtagtgcggcatctaggtgttactaaaggcatattgacaggctgttttagttttgccaagatgttatttatgcttccac |
| | | tagcttactttagtgattcaaaactcggcaccacagaggttaaagtgagtgattgaaaacagccggcgttgtgacaggtaatgttgtaaaacagtgttg |
| | | cactgctgctgttgatttaagtatggataagttgcgccgtgtggattggaaatcaaccctacggttgttacttatgttatgcacaactatggtattgtt |
| | | gtcttctgtgtatcacttgtatgtcttcaatcaggtcttatcaagtgatgttatgtttgaagatgcccaaggtttgaaaaagttctacaaagaagttag |
| | | agcttacctaggaatctcttctgcttgtgacggtcttgcttcagcttatagggcgaattcctttgatgtacctacattctgcgcaaaccgttctgcaat |
| | | gtgtaattggtgcttgattagccaagattccataactcactacccagctcttaagatggttcaaacacatcttagccactatgttcttaacatagattg |
| | | gttgtggtttgcatttgagactggtttggcatacatgctctatacctcggccttcaactggttgttgttggcaggtacattgcattatttctttgcaca |
| | | gacttccatatttgtagactggcggtcatacaattatgctgtgtctagtgccttctggttattcacccacattccaatggcgggtttggtacgaatgta |
| | | taatttgttagcatgcctttggcttttacgcaagttttatcagcatgtaatcaatggttgcaaagatacggcatgcttgctctgctataagaggaaccg |
| | | acttactagagttgaagcttctaccgttgtctgtggtggaaaacgtacgttttatatcacagcaaatggcggtatttcattctgtcgtaggcataattg |
| | | gaattgtgtggattgtgacactgcaggtgtggggaataccttcatctgtgaagaagtcgcaaatgacctcactaccgccctacgcaggcctattaacgc |
| | | tacggatagatcacattattatgtggattccgttacagttaaagagactgttgttcagtttaattatcgtagagacggtcaaccattctacgagcggtt |
| | | tcccctctgcgcttttacaaatctagataagttgaagttcaaagaggtctgtaaaactactactggtatacctgaatacaactttatcatctacgactc |
| | | atcagatcgtggccaggaaagtttagctaggtctgcatgtgtttattattctcaagtcttgtgtaaatcaattcttttggttgactcaagtttggttac |
| | | ttctgttggtgattctagtgaaatcgccactaaaatgtttgattcctttgttaatagtttcgtctcgctgtataatgtcacacgcgataagttggaaaa |
| | | acttatctctactgctcgtgatggcgtaaggcgaggcgataacttccatagtgtcttaacaacattcattgacgcagcacgaggccccgcaggtgtgga |
| | | gtctgatgttgagaccaatgaaattgttgactctgtgcagtatgctcataaacatgacatacaaattactaatgagagctacaataattatgtaccctc |
| | | atatgttaaacctgatagtgtgtctaccagcgatttaggtagtctcattgattgtaatgcggcttcagttaaccaaattgtcttgcgtaattctaatgg |
| | | tgcttgcatttggaacgctgctgcatatatgaaactctcggatgcacttaaacgacagattcgcattgcatgccgtaagtgtaatttagctttccggtt |
| | | aaccacctcaaagctacgcgctaatgataatatcttatcagttagattcactgctaacaaaattgttggtggtgctcctacatggtttaatgcgttgcg |
| | | tgactttacgttaaagggttatgttcttgctaccattattgtgtttctgtgtgctgtactgatgtatttgtgtttacctacattttctatggcacctgt |
| | | tgaattttatgaagaccgcatcttggactttaaagttcttgataatggtatcattagggatgtaaatcctgatgataagtgctttgctaataagcaccg |
| | | gtccttcacacaatggtatcatgagcatgttggtggtgtctatgacaactctatcacatgcccattgacagttgcagtaattgctggagttgctggtgc |
| | | tcgcattccagacgtacctactacattggcttgggtgaacaatcagataattttattgtttctcgagtctttgctaatacaggcagtgtttgctacact |
| | | cctatagatgagataccctataagagtttactgatagtggttgcattcttccatctgagtgcactatgtttagggatgcagagggccgtatgacaccat |
| | | actgccatgatcctactgttttgcctggggcttttgcgtacagtcagatgaggcctcatgttcgttacgacttgtatgatggtaacatgtttattaaat |
| | | ttcctgaagtagtatttgaaagtacacttaggattactagaactctgtcaactcagtactgccggttcggtagttgtgagtatgcacaagagggtgttt |
| | | gtattaccacaaatggctcgtgggccatttttaatgaccaccatcttaatagacctggtgtctattgtggctctgattttattgacattgtcaggcggt |
| | | tagcagtatcactgttccagcctattacttatttccaattgactacctcattggtcttgggtataggtttgtgtgcgttcctgactttgctcttctatt |
| | | atattaataaagtaaaacgtgatttgcagattacacccagtgtgctgtaattgctgttgttgctgctgttcttaatagcttgtgcatctgctttgttac |
| | | ctctataccattgtgtatagtaccttacactgcattgtactattatgctacattctattttactaatgagcctgcatttattatgcatgtttcttggta |
| | | cattatgttcgggcctatcgttcccatatggatgacctgcgtctatacagttgcaatgtgattagacacttcttctgggttttagcttattttagtaag |
| | | aaacatgtagaagtttttactgatggtaagcttaattgtagtttccaggacgctgcctctaatatctttgttattaacaaggacacttatgcagctctt |
| | | agaaactctttaactaatgatgcctattcacgatttttggggttgtttaacaagtataagtacttactggtgctatggaaacagccgcttatcgtgaag |
| | | ctgcagcatgtcatcttgctaaagccttacaaacatacagcgagactggtagtgatcttctttaccaaccacccaactgtagcataacctaggcgtgtt |
| | | gcaaagcggtttggtgaaaatgtcacatcccagtggagatgttgaggcttgtatggttcaggttacctgcggtagcatgactcttaatggtctttggct |
| | | tgacaacacagtctggtgcccacgacacgtaatgtgcccggctgaccagttgtctgatcctaattatgatgccttgttgatttctatgactaatcatag |
| | | tttcagtgtgcaaaaacacattggcgctccagcaaacttgcgtgttgttggtcatgccatgcaaggcactcttttgaagttgactgtcgatgttgctaa |
| | | ccctagcactccagcctacacttttacaacagtgaaacctggcgcagcatttagtgtgttagcatgctataatggtcgtccgactggtacattcactgt |
| | | tgtaatgcgccctaactacacaattaagggttcctttctgtgtggttcttgtggtagtgttggttacaccaaggagggtagtgtgatcaatttctgtta |
| | | catgcatcaaatggaacttgctaatggtacacataccggttcagcatttgatggtactatgtatggtgcctttatggataaacaagtgcaccaagttca |
| | | gttaacagacaaatactgcagtgttaatgtagtagcttggctttacgcagcaatacttaatggttgcgcttggtttgtaaaacctaatcgcactagtgt |
| | | tgtttcttttaatgaatgggctcttgccaaccaattcactgaatttgttggcactcaatccgttgacatgttagctgtcaaaacaggcgttgctattga |
| | | acagctgattatgcgatccaacaactgtatactgggttccagggaaagcaaatccttggcagtaccatgttggaagatgaattcacacctgaggatgtt |
| | | aatatgcagattatgggtgtggttatgcagagtggtgtgagaaaagttacatatggtactgcgcattggttgtttgcgacccttgtctcaacctatgtg |
| | | ataatcttacaagccactaaatttactttgtggaactacttgtttgagactattcccacacagttgttcccactcttatttgtgactatggccttcgtt |
| | | atgttgttggttaaacacaaacacacattttgacacttttcttgttgcctgtggctatttgtttgacttatgcaaacatagtctacgagcccactactc |
| | | ccatttcgtcagcgctgattgcagttgcaaattggcttgcccccactaatgcttatatgcgcactacacatactgatattggtgtctacattagtatgt |
| | | cacttgtattagtcattgtagtgaagagattgtacaacccatcactttctaactttgcgttagcattgtgcagtggtgtaatgtggttgtacacttata |
| | | gcattggagaagcctcaagccccattgcctatctggtttttgtcactacactcactagtgattatacgattacagtctttgttactgtcaaccttgcaa |
| | | aagtttgcacttatgccatattgcttactcaccacagcttacacttgtgt-ttccggaagtgaagatgatacttttattatacacatgtttaggtttca |
| | | tgtgtacttgctattttggtgtcttctctcttttgaaccttaagcttagagcacctatgggtgtctatgactttaaggtctcaacacaagagttcagat |
| | | tcatgactgctaacaatctaactgcacctagaaattcttgggaggctatggctctgaactttaagttaataggtattggcggtacaccttgtataaagg |
| | | ttgctgctatgcagtctaaacttacagatcttaaatgcacatctgtggttctcctctctgtgctccaacagttacacttagaggctaatagtagggcct |
| | | gggctttctgtgttaaatgccataatgatatattggcagcaacagaccccagtgaggctttcgagaaattcgtaagtctattgctactttaatgacttt |
| | | ttctggtaatgtagatcttgatgcgttagctagtgatatttttgacactcctagcgtacttcaagctactctttctgagttttcacacttagctacctt |
| | | tgctgagttggaagctgcgcagaaagcctatcaggaagctatggactctggtgacacctcaccacaagttcttaaggctttgcagaaggctgttaatat |
| | | agctaaaaacgcctatgagaaggataaggcagtggcccgtaagttagaacgtatggctgatcaggctatgacttctatgtataagcaagcacgtgctga |
| | | agacaagaaagcaaaaattgtcagtgctatgcaaactatgttgtttggtatgattaagaagctcgacaacgatgttcttaatggtatcatttctaacgc |
| | | taggaatggttgtatacctcttagtgtcatcccactgtgtgcttcaaataaacttcgcgttgtaattcctgacttcaccgtctggaatcaggtagtcac |
| | | atatccctcgcttaactacgctggggctttgtgggacattacagttataaacaatgtggacaatgaaattgttaagtcttcagatgttgtagacagcaa |
| | | tgaaaatttaacatggccacttgttttagaatgcactagggcatccacttctgccgttaagttgcaaaataatgagatcaaaccttcaggtctaaaaac |
| | | catggttgtgtctgcgggtcaagagcaaactaactgtaatactagttccttagcttattacgaacctgtgcagggtcgtaaaatgctgatggctcttct |
| | | ttctgataatgcctatctcaaatgggcgcgtgttgaaggtaaggacggatttgtcagtgtagagctacaacctccttgcaaattcttgattgcgggacc |
| | | aaaaggacctgaaatccgatatctctattttgttaaaaatcttaacaaccttcatcgcgggcaagtgttagggcacattgctgcgactgttagattgca |
| | | agctggttctaacaccgagtttgcctctaattcctcggtgttgtcacttgttaacttcaccgttgatcctcaaaaagcttatctcgatttcgtcaatgc |
| | | gggaggtgccccattgacaaattgtgttaagatgcttactcctaaaactggtacaggtatagctatatctgttaaaccagagagtacagctgatcaaga |
| | | gacttatggtggagcttcagtgtgtactattgccgtgcgcatatagaacatcctgatgtactggtgtttgtaaatataagggtaagtttgtccaaatcc |
| | | ctgctcagtgtgtccgtgaccctgtgggattttgtttgtcaaataccccctgtaatgtagtcaatattggattggatatgggtgcaattgtgactcgct |
| | | taggcaagcagcactgccccaatctaaagattccaattttttaaacgagtccggggttctattgtaaatgcccgaatagaaccctgttcaagtggtttg |
| | | tccactgatgtcgtctttagggcatttgacatctgcaactataaggctaaggttgctggtattggaaaatactacaagactaatacttgtaggtttgta |
| | | gaattagatgaccaagggcatcatttagactcctattttgtcgttaagaggcatactatggagaattatgaactagagaagcactgttacgacttgtta |
| | | cgtgactgtgatgctgtagctccccatgatttcttcatctttgatgtagacaaagttaaaacacctcatattgtacgtcagcgtttaactgagtacact |
| | | atgatggatcttgtatatgccctgaggcactttgatcaaaatagcgaagtgcttaaggctatcttagtgaagtatggttgctgtgatgttacctacttt |
| | | gaaaataaactaggtttgattttgttgaaaatcccagtgttattggtgtttatcataaacttggagaacgtgtacgccaagctatcttaaacactgtta |
| | | aattttgtgaccacatggtcaaggctggtttagtcggtgtgctcacactagacaaccaggaccttaatggcaagtggtatgattttggtgacttcgtaa |
| | | tcactcaacctggttcaggagtagctatagttgatagctactattcttatttgatgcctgtgctctcaatgaccgattgtctggccgctgagacacata |
| | | gggattgtgattttaataaaccactcattgagtggccacttactgagtatgattttactgattataaggtacaactctttgagaagtactttaaatatt |
| | | gggatcagacgtatcacgcaaattgcgttaattgtactgatgaccgttgtgtgttacattgtgctaatttcaatgtattgtttgctatgaccatgccta |
| | | agacttgtttcggacccatagtccgaaagatctttgttgatggcgtgccatttgtagtatcttgtggttatcactacaaagaattaggtttagtcatga |
| | | atatggatgttagtctccatagacataggctctctcttaaggagttgatgatgtatgccgctgatccagccatgcacattgcctcctctaacgcttttc |
| | | ttgatttgaggacatcatgttttagtgtcgctgcacttacaactggtttgacttttcaaactgtgcggcctggcaattttaaccaagacttctatgatt |
| | | tcgtggtatctaaaggtttctttaaggagggctcttcagtgacgctcaaacattttttctttgctcaagatggtaatgctgctattacagattataatt |
| | | actattcttataatctgcctactatgtgtgacatcaaacaaatgttgttctgcatggaagttgtaaacaagtacttcgaaatctatgacggtggttgtc |
| | | ttaatgcttctgaagtggttgttaataatttagacaagagtgctggccatccttttaataagtttggcaaagctcgtgtctattatgagagcatgtctt |
| | | accaggagcaagatgaactttttgccatgacaaagcgtaacgtcattcctaccatgactcaaatgaatctaaaatatgctattagtgctaagaatagag |
| | | ctcgcactgttgcaggcgtgtccatacttagcacaatgactaatcgccagtaccatcagaaaatgcttaagtccatggctgcaactcgtggagcgactt |
| | | gcgtcattggtactacaaagttctacggtggctgggatttcatgcttaaaacattgtacaaagatgttgataatccgcatcttatgggttgggattacc |
| | | ctaagtgtgatagagctatgcctaatatgtgtagaatcttcgcttcactcatattagctcgtaaacatggcacttgttgtactacaagggacagatttt |
| | | atcgcttggcaaatgagtgtgctcaggtgctaagcgaatatgttctatgtggtggtggttactacgtcaaacctggaggtaccagtagcggagatgcca |
| | | ccactgcatatgccaatagtgtctttaacattttgcaggcgacaactgctaatgtcagtgcacttatgggtgctaatggcaacaagattgttgacaaag |
| | | aagttaaagacatgcagtttgatttgtatgtcaatgtttacaggagcactagcccagaccccaaatttgttgataaatactatgatttcttaataagca |
| | | cttttctatgatgatactgtctgatgacggtgtcgtttgctataatagtgattatgcagctaagggttacattgctggaatacagaattttaaggaaac |
| | | gctgtattatcagaacaatgtctttatgtctgaagctaaatgctgggtggaaaccgatctgaagaaagggccacatgaattctgttcacagcatacgat |
| | | tatattaaggatggcgacgatggttacttccttccttatccagacccttcaagaattttgtctgccggttgattgtagatgatatcgttaagactgacg |
| | | gtacactcatggtagagcggtttgtgtctttggctatagatgcttaccctctcacaaagcatgaagatatagaataccagaatgtattctgggtctact |
| | | tacagtatatagaaaaactgtataaagaccttacaggacacatgcttgacagttattctgtcatgctatgtggtgataattctgctaagttttgggaag |
| | | aggcattctatagagatctctatagttcgcctaccactttgcaggctgtcggttcatgcgttgtatgccattcacagacttccctacgctgtgggacat |
| | | gcatccgtagaccatttctctgctgtaaatgctgctatgatcatgttatagcaactccacataagatggttttgtctgtttctccttacgtttgtaatg |
| | | cccctggttgtggcgtttcagacgttactaagctatatttaggtggtatgagctacttttgtgtagatcatagacctgtgtgtagtatccactttgcgc |
| | | taatggtcttgtattcggcttatacaagaatatgtgcacaggtagtccttctatagttgaatttaataggttggctacctgtgactggactgaaagtgg |
| | | tgattacacccttgccaatactacaacagaaccactcaaactttttgctgctgagactttacgtgccactgaagaggcgtctaagcagtcttatgctat |
| | | tgccaccatcaaagaaattgttggtgagcgccaactattacttgtgtgggaggctggcaagtccaaaccaccactcaatcgtaattatgtttttactgg |
| | | ttatcatataaccaaaaatagtaaagtgcagctcggtgagtacattttcgagcgcattgattatagtgatgctgtatcctacaagtctagtacaacgta |
| | | taaactgactgtaggtgacatcttcgtacttacctctcactctgtggctaccttgacggcgcccacaattgtgaatcaagagaggtatgttaaaattac |
| | | tgggttgtacccaaccattacggtacctgaagagttcgcaagtcatgttgccaacttccaaaaatcaggttatagtaaatatgtcactgttcagggacc |
| | | acctggcactggcaaaagtcattttgctatagggttagcgatttactaccctacagcacgtgttgtttatacagcatgttcacacgcagctgttgatga |
| | | ttgtgtgaaaaagcttttaaatatttgaacattgctaaatgttcccgtatcattcctgcaaaggcacgtgttgagtgctatgacaggtttaaagttaat |
| | | gagacaaattctcaatatttgtttagtactattaatgactaccagaaacttctgccgatattctggtggttgatgaggttagtatgtgcactaattatg |
| | | atctttcaattattaatgcacgtattaaagctaagcacattgtctatgtaggagatccagcacagttgccagctcctaggactttgttgactagaggca |
| | | cattggaaccagaaaatttcaatagtgtcactagattgatgtgtaacttaggtcctgacatatttttaagtatgtgctacaggtgtcctaaggaaatag |
| | | taagcactgtgagcgctcttgtctacaataataaattgttagccaagaaggagctttcaggccagtgattaaaatactctataagggcaatgtgacgca |
| | | tgatgctagctctgccattaatagaccacaactcacatttgtgaagaattttattactgccaatccggcatggagtaaggcagtctttatttcgcctta |
| | | caattcacagaatgctgtgtctcgttcaatgctgggtcttaccactcagactgttgattcctcacagggttcagaataccagtacgttatcttctgtca |
| | | aacagcagatacggcacatgctaacaacattaacagatttaatgttgcaatcactcgtgcccaaaaaggtattctttgtgttatgacatctcaggcact |
| | | ctttgagtccttagagtttactgaattgtcttttactaattacaagctccagtctcagattgtaactggcctttttaaagattgactagagaaacttct |
| | | ggcctctcacctgcttatgcaccaacatatgttagtgttgatgacaagtataagacgagtgatgagctttgcgtgaatcttaatttacccgcaaatgtc |
| | | ccatactctcgtgttatttccaggatgggctttaaactcgatgcaacagttcctggatatcctaagcttttcattactcgtgaagaggctgtaaggcaa |
| | | gttcgaagaggataggcttcgatgttgagggtgctcatgcttcccgtaatgcatgtggcaccaatgtgcctctacaattaggattttcaactggtgtga |
| | | actttgttgttcagccagttggtgttgtagacactgagtggggtaacatgttaacgggcattgctgcacgtcctccaccaggtgaacagtttaagcacc |
| | | tcgtgcctcttatgcataagggggctgcgtggcctattgttagacgacgtatagtgcaaatgttgtcagacactttagacaaattgtctgattactgta |
| | | cgtttgtttgttgggctcatggctttgaattaacgtctgcatcatacttttgcaagataggtaaggaacagaagtgttgcatgtgcaatagacgcgctg |
| | | cagcgtactcttcacctctgcaatcttatgcctgctggactcattcctgcggttatgattatgtctacaaccattctttgtcgatgttcaacagtgggg |
| | | ttatgtaggcaatcttgctactaatcacgatcgttattgctctgtccatcaaggagctcatgtggcttctaatgatgcaataatgactcgttgtttagc |
| | | tattcattcttgttttatagaacgtgtggattgggatatagagtatccttatatctcacatgaaaagaaattgaattcctgttgtagaatcgttgagcg |
| | | caacgtcgtacgtgctgctcttcttgccggttcatttgacaaagtctatgatattggcaatcctaaaggaattcctattgttgatgaccagtggttgat |
| | | tggcattattttgatgcacagcccttgaccaggaaggtacaacagcttttctatacagaggacatggcctcaagatttgctgatgggctctgcttattt |
| | | tggaactgtaatgtaccaaaatatcctaataatgcaattgtatgcaggtttgacacacgtgtgcattctgagttcaatttgccaggttgtgatggcggt |
| | | agtttgtatgttaacaagcacgcttttcatacaccagcatatgatgtgagtgcattccgtgatctgaaacctttaccattcttttattattctactaca |
| | | ccatgtgaagtgcatggtaatggtagtatgatagaggatattgattatgtacccctaaaatctgcagtagtattacagcttgtaatttagggggcgctg |
| | | tttgtaggaagcatgctacagagtacagagagtatatggaagcatataatcttgtactgcatcaggtttccgcctttggtgttataagacctttgatat |
| | | ttataatctctggtctacttttacaaaagttcaaggtttggaaaacattgcttttaatgttgttaaacaaggccattttattggtgttgagggtgaact |
| | | acctgtagagtagtcaatgataagatcttcaccaagagtggcgttaatgacatttgtatgtttgagaataaaaccactttgcctactaatatagctttt |
| | | gaactctatgctaagcgtgctgtacgctcgcatcccgatttcaaattgctacacaatttacaagcagacatttgctacaagttcgtcctttgggattat |
| | | gaacgtagcaatatttatggtactgctactattggtgtatgtaagtacactgatattgatgttaattcagctttgaatatatgttttgacatacgcgat |
| | | aattgttcattggagaagttcatgtctactcccaatgccatctttatttctgatagaaaaatcaagaaatacccttgtatggtaggtcctgattatgct |
| | | tacttcaatggtgctatcatccgtgatagtgatgttgttaaacaaccagtgaagttctacttgtataagaaagtcaataatgagtttattgatcctact |
| | | gagtgtatttacactcagagtcgctcttgtagtgacttcctacccattctgacatggagaaagactttctatatttgatagtgatgttttcattaagaa |
| | | gtatggcttggaaaactatgatttgagcacgtagtctatggagacttctctcatactacgttaggcggtcttcacttgcttattggtttatacaagaag |
| | | caacaggaaggtcatattattatggaagaaatgctaaaaggtagctcaactattcataactattttattactgagactaacacagcggcttttaaggcg |
| | | gtgtgttctgttatagatttaaagcttgacgactttgttatgattttaaagagtcaagaccttggcgtagtatccaaggttgtcaaggttcctattgac |
| | | ttaacaatgattgagtttatgttatggtgtaaggatggacaggttcaaaccttctaccctcgactccaggcttctgcagattggaaacctggtcatgca |
| | | atgccatccctctttaaagttcaaaatgtaaaccttgaacgttgtgagcttgctaattacaagcaatctattcctatgcctcgcggtgtgcacatgaac |
| | | atcgctaaatatatgcaattgtgccagtatttaaatacttgcacattagccgtgcctgccaatatgcgtgttatacattttggcgctggttctgataaa |
| | | ggtatcgctcctggtacctcagttttacgacagtggcttcctacagatgccattattatagataatgatttaaatgagttcgtgtcagatgctgacata |
| | | actttatttggagattgtgtaactgtacgtgtcggccaacaagtggatcttgttatttccgacatgtatgatcctactactaagaatgtaacaggtagt |
| | | aatgagtcaaaggctttattattacttacctgtgtaacctcattaataataatcttgctcttggtgggtctgttgctattaaaataacagaacactctt |
| | | ggagcgttgaactttatgaacttatgggaaaatttgcttggtggactgttttctgcaccaatgcaaatgcatcctcatctgaaggattcctcttaggta |
| | | ttaattacttgggtactattaaagaaaatatagatggtggtgctatgcacgccaactatatattttggagaaattccactcctatgaatctgagtactt |
| | | actcactttttgatttatccaagtttcaattaaaattaaaaggaacaccagttcttcaattaaaggagagtcaaattaacgaactcgtaatatctctcc |
| | | tgtcgcagggtaagttacttatccgtgacaatgatacactcagtgtttctactgatgttcttgttaacacctacagaaagttacgttgatgtagggcca |
| | | gattctgttaagtagcttgtattgaggttgatatacaacagactttattgataaaacttggcctaggccaattgatgtttctaaggctgacggtattat |
| | | ataccctcaaggccgtacatattctaacataactatcacttatcaaggtctttttccctatcagggagaccatggtgatatgtatgtttactctgcagg |
| | | acatgctacaggcacaactccacaaaagttgtttgtagctaactattctcaggacgtcaaacagtttgctaatgggtttgtcgtccgtataggagcagc |
| | | tgccaattccactggcactgttattattagcccatctaccagcgctactatacgaaaaatttaccctgatttatgctgggttcttcagttggtaatttc |
| | | tcagatggtaaaatgggccgcttcttcaatcatactctagttcttttgcccgatggatgtggcactttacttagagctttttattgtattctagagcct |
| | | cgctctggaaatcattgtcctgctggcaattcctatacttatttgccacttatcacactcctgcaacagattgttctgatggcaattacaatcgtaatg |
| | | ccagtctgaactcttttaaggagtattttaatttacgtaactgcacctttatgtacacttataacattaccgaagatgagattttagagtggtttggca |
| | | ttacacaaactgctcaaggtgttcacctcttctcatctcggtatgttgatttgtacggcggcaatatgtttcaatttgccaccttgcctgtttatgata |
| | | ctattaagtattattctatcattcctcacagtattcgttctatccaaagtgatagaaaagcttgggctgccttctacgtatataaacttcaaccgttaa |
| | | ctttcctgttggatttttctgttgatggttatatacgcagagctatagactgtggttttaatgatttgtcacaactccactgctcatatgaatccttcg |
| | | atgttgaatctggagtttattcagtttcgtctttcgaagcaaaaccttctggctcagttgtggaacaggctgaaggtgttgaatgtgatttttcacctc |
| | | ttctgtaggcacacctcctcaggtttataatttcaagcgtttggtttttaccaattgcaattataatcttaccaaattgattcacttttttctgtgaat |
| | | gattttacttgtagtcaaatatctccagcagcaattgctagcaactgttattcttcactgattttggattacttttcatacccacttagtatgaaatcc |
| | | gatctcagtgttagttctgctggtccaatatcccagtttaattataaacagtccttttctaatcccacatgtttgattttagcgactgttcctcataac |
| | | cttactactattactaagcctcttaagtacagctatattaacaagtgactcgtcttattctgatgatcgtactgaagtacctcagttagtgaacgctaa |
| | | tcaatactcaccctgtgtatccattgtcccatccactgtgtgggaagacggtgattattataggaaacaactatctccacttgaaggtggtggctggct |
| | | tgttgctagtggctcaactgttgccatgactgagcaattacagatgggctttggtattacagttcaatatggtacagacaccaatagtgtttgccccaa |
| | | gcttgaatttgctaatgacacaaaaattgcctctcaattaggcaattgcgtggaatattccctctatggtgtttcgggccgtggtgtttttcagaattg |
| | | cacagctgtaggtgttcgacagcagcgctttgtttatgatgcgtaccagaatttagttggctattattctgatgatggcaactactactgtttgcgtgc |
| | | ttgtgttagtgttcctgtttctgtcatctatgataaagaaactaaaacccacgctactctatttggtagtgttgcatgtgaacacatttcttctaccat |
| | | gtctcaatactcccgttctacgcgatcaatgcttaaacggcgagattctacatatggccccdtcagacacctgttggttgtgtcctaggacttgttaat |
| | | tcctctttgttcgtagaggactgcaagttgcctcttggtcaatctctctgtgctcttcctgacacacctagtactctcacacctcgcagtgtgcgctct |
| | | gttccaggtgaaatgcgcttggcatccattgatttaatcatcctattcaggttgatcaacttaatagtagttattttaaattaagtatacccactaatt |
| | | tttcctttggtgtgactcaggagtacattcagacaaccattcagaaagttactgttgattgtaaacagtacgtttgcaatggtttccagaagtgtgagc |
| | | aattactgcgcgagtatggccagttttgttccaaaataaaccaggctctccatggtgccaatttacgccaggatgattctgtacgtaatttgtttgcga |
| | | gcgtgaaaagctctcaatcatctcctatcataccaggttttggaggtgactttaatttgacacttctagaacctgtttctatatctactggcagtcgta |
| | | gtgcacgtagtgctattgaggatttgctatttgacaaagtcactatagctgatcctggttatatgcaaggttacgatgattgcatgcagcaaggtccag |
| | | catcagctcgtgatcttatttgtgctcaatatgtggctggttacaaagtattacctcctcttatggatgttaatatggaagccgcgtatacttcatctt |
| | | tgcttggcagcatagcaggtgttggctggactgctggcttatcctcctttgctgctattccatttgcacagagtatcttttataggttaaacggtgttg |
| | | gcattactcaacaggttattcagagaaccaaaagcttattgccaataagtttaatcaggctctgggagctatgcaaacaggcttcactacaactaatga |
| | | agatttcagaaggttcaggatgctgtgaacaacaatgcacaggctctatccaaattagctagcgagctatctaatacttttggtgctatttccgcctct |
| | | attggagacatcatacaacgtcttgatgttctcgaacaggacgcccaaatagacagacttattaatggccgtttgacaacactaaatgatttgttgcac |
| | | agcagcttgttcgttccgaatcagctgctctttccgctcaattggctaaagataaagtcaatgagtgtgtcaaggcacaatccaagcgttctggatttt |
| | | gcggtcaaggcacacatatagtgtcctttgttgtaaatgcccctaatggcctttacttcatgcatgttggttattaccctagcaaccacattgaggttg |
| | | tttctgcttatggtctttgcgatgcagctaaccctactaattgtatagcccctgttaatggctactttattaaaactaataacactaggattgttgatg |
| | | agtggtcatatactggctcgtccttctatgcacctgagcccattacctcccttaatactaagtatgttgcaccacaggtgacataccaaaacatttcta |
| | | ctaacctccctcctcctcttctcggcaattccaccgggattgacttccaagatgagttggatgagtttttcaaaaatgttagcaccagtatacctaatt |
| | | ttggttccctaacacagattaatactacattactcgatcttacctacgagatgttgtctcttcaacaagttgttaaagcccttaatgagtcttacatag |
| | | accttaaagagcttggcaattatacttattacaacaaatggccgtggtacatttggcttggtttcattgctgggcttgttgccttagctctatgcgtct |
| | | tcttcatactgtgctgcactggttgtggcacaaactgtatgggaaaacttaagtgtaatcgttgttgtgatagatacgaggaatacgacctcgagccgc |
| | | ataaggttcatgttcactaattaacgaactattaatgagagttcaaagaccacccactctcttgttagtgttttcactctctcttttggtcactgcatc |
| | | ctcaaaacctctctatgtacctgagcattgtcagaattattctggttgcatgcttagggcttgtattaaaactgcccaagctgatacagctggtattat |
| | | acaaattttcgaattgacgtcccatctgcagaatcaactggtactcaatcagtttctgtcgatcttgagtcaacttcaactcatgatggtcctaccgaa |
| | | catgttactagtgtgaatctttttgacgttggttactcagttaattaacgaactctatggattacgtgtctctgcttaatcaaatttggcagaagtacc |
| | | ttaactcaccgtatactacttgtttgtacatccctaaacccacagctaagtatacacctttagttggcacttcattgcaccctgtgctgtggaactgtc |
| | | agctatcctttgctggttatactgaatctgctgttaattctacaaaagctttggccaaacaggacgcagctcagcgaatcgcttggttgctacataagg |
| | | atggaggaatccctgatggatgttccctctacctccggcactcaagtttattcgcgcaaagcgaggaagaggagccattaccaactaagaaactgcgct |
| | | acgttaagcgtagattttctcttctgcgccatgaagaccttagtgttattgtccaaccaacacactatgtcagggttacattttcagaccccaacatgt |
| | | ggtatctacgttcgggtcatcatttacactcagttcacaattggcttaaaccttatggcggccaacctgtttctgagtaccatattactctagctttgc |
| | | taaatctcactgatgaagatttagctagagatttttcacccattgcgctctttttgcgcaatgtcagatttgagctacatgagttcgccttgctgcgca |
| | | aaactcttgttcttaatgcatcagagatctactgtgctaacatacatagatttaagcctgtgtatagagttaacacggcaatccctactattaaggatt |
| | | ggcttctcgttcagggattttccattaccatagtggcctccctttacatatgtcaatctctaaattgcatgcactggatgatgttactcgcaattacat |
| | | cattacaatgccatgctttagaacttaccctcaacaaatgtttgttactcctttggccgtagatgttgtaccatacggtcttccaatcagggtaataaa |
| | | caaattgttcattcttatcccattttacatcatccaggattttaacgaactatggctttctcggcgtattatttaaacccgtccagctagtcccagttt |
| | | ctcctgcatttcatcgcattgagtctactgactctattgttttcacatacattcctgctagcggctatgtagctgattagctgtcaatgtgtgtctcat |
| | | tcccctattattactgctacgtcaagatacttgtcgtcgcagcattatcagaactatggttctctatttccttgttctgtataactttttattagccat |
| | | tgtactagtcaatggtgtacattatccaactggaagttgcctgatagccttcttagttatcctcataatactttggtttgtagatagaattcgtttctg |
| | | tctcatgctgaattcctacattccactgtttgacatgcgttcccactttattcgtgttagtacagtttcttctcatggtatggtccctgtaatacacac |
| | | caaaccattatttattagaaacttcgatcagcgttgcagctgttctcgttgtttttatttgcactcttccacttatatagagtgcacttatattagccg |
| | | ttttagtaagattagcctagtttctgtaactgacttctccttaaacggcaatgtttccactgttttcgtgcctgcaacgcgcgattcagttcctcttca |
| | | cataatcgccccgagctcgcttatcgtttaagcagctctgcgctactatgggtcccgtgtagaggctaatccattagtctctctttggacatatggaaa |
| | | acgaactatgttaccattgtccaagaacgaatagggttgttcatagtaaactttttcatttttaccgtagtatgtgctataacactcttggtgtgtatg |
| | | gctttccttacggctactagattatgtgtgcaatgtatgacaggcttcaataccctgttagttcagcccgcattatacttgtataatactggacgttca |
| | | gtctatgtaaaattccaggatagtaaaccccctctaccacctgacgagtgggtttaacgaactccttcataatgtctaatatgacgcaactcactgagg |
| | | cgcagattattgccattattaaagactggaactttgcatggtccctgatctttctcttaattactatcgtactacagtatggatacccatcccgtagta |
| | | tgactgtctatgtattaaaatgtttgttttatggctcctatggccatcttccatggcgctatcaatatttagcgccgtttatccaattgatctagcttc |
| | | ccagataatctctggcattgtagcagctgtttcagctatgatgtggatttcctactttgtgcagagtatccggctgtttatgagaactggatcatggtg |
| | | gtcattcaatcctgagactaattgccttttgaacgttccatttggtggtacaactgtcgtacgtccactcgtagaggactctaccagtgtaactgctgt |
| | | tgtaaccaatggccacctcaaaatggctggcatgcatttcggtgcttgtgactacgacagacttcctaatgaagtcaccgtggccaaacccaatgtgct |
| | | gattgattaaaaatggtgaagcggcaaagctacggaactaattccggcgttgccatttaccatagatataaggcaggtaattacaggagtccgcctatt |
| | | acggcggatattgaacttgcattgcttcgagcttaggctattagtaagagtatcttaattgattttaacgaatctcaatttcattgttatggcatcccc |
| | | tgctgcacctcgtgctgtttcctttgccgataacaatgatataacaaatacaaacctatctcgaggtagaggacgtaatccaaaaccacgagctgcacc |
| | | aaataacactgtctcttggtacactgggcttacccaacacgggaaagtccctcttacctttccacctgggcagggtgtacctcttaatgccaattctac |
| | | ccctgcgcaaaatgctgggtattggcggagacaggacagaaaaattaataccgggaatggaattaagcaactggctcccaggtggtacttctactacac |
| | | tggaactggacccgaagcagcactcccattccgggctgttaaggatggcatcgtttgggtccatgaagatggcgccactgatgctccttcaacttttgg |
| | | gacgcggaaccctaacaatgattcagctattgttacacaattcgcgcccggtactaagcttcctaaaaacttccacattgaggggactggaggcaatag |
| | | tcaatcatcttcaagagcctctagcttaagcagaaactcttccagatctagttcacaaggttcaagatcaggaaactctacccgcggcacttaccaggt |
| | | ccatctggaatcggagcagtaggaggtgatctactttaccttgatcttctgaacagactacaagcccttgagtaggcaaagtaaagcaatcgcagccaa |
| | | aagtaatcactaagaaagatgctgctgctgctaaaaataagatgcgccacaagcgcacttccaccaaaagtttcaacatggtgcaagcttttggtcttc |
| | | gcggaccaggagacctccagggaaactttggtgatcttcaattgaataaactcggcactgaggacccacgttggccccaaattgctgagcttgctccta |
| | | cagccagtgcttttatgggtatgtcgcaatttaaacttacccatcagaacaatgatgatcatggcaaccctgtgtacttccttcggtacagtggagcca |
| | | ttaaacttgacccaaagaatcccaactacaataagtggttggagatcttgagcaaaatattgatgcctacaaaaccttccctaagaaggaaaagaaaca |
| | | aaaggcaccaaaagaagaatcaacagaccaaatgtctgaacctccaaaggagcagcgtgtgcaaggtagcatcactcagcgcactcgcacccgtccaag |
| | | tgttcagcctggtccaatgattgatgttaacactgattagtgtcactcaaagtaacaagatcgcggcaatcgtttgtgtttggcaaccccatctcacca |
| | | tcgcttgtccactatgcacagaatggaatcatgttgtaattacagtgcaataaggtaattataacccatttaattgatagctatgctttattaaagtgt |
| | | gtagctgtagagagaatgttaaagactgtcacctctgcttgattgcaagtgaacagtgccccccgggaagagctctacagtgtgaaatgtaaataaaaa |
| | | atagctattattcaattagattaggctaattagatgatttgcaaaaaaaaaaaa |
| 5538 | hCoV-OC43 genome | (1) attgtgagcgatttgcgtgcgtgcatcccgcttcactgatctcttgttagatctttttgtaatctaaactttataaaaacatccactccctgtaa |
| | Genbank Accession No. NC_006213.1 | tctatgcttgtgggcgtagatttttcatagtggtgtttatattcatttctgctgttaacagattcagccagggacgtgttgtatcctaggcagtggccc |
| | | gcccataggtcacaatgtcgaagatcaacaaatacggtctcgaactacactgggctccagaatttccatggatgtttgaggacgcagaggagaagttgg |
| | | ataaccctagtagttcagaggtggatatgatttgctccaccactgcgcaaaagctggaaacagacggaatttgtcctgaaaatcatgtgatggtggatt |
| | | gtcgccgacttcttaaacaagagtgttgtgtgcagtctagcctaatacgtgaaattgttatgaatgcaagtccatatgatttggaggtgctacttcaag |
| | | atgctttgcagtcccgtgaagcagttttggttacaacccccttaggtatgtctttagaggcatgctatgtgagaggttgtaatcctaaaggatggacca |
| | | tgggtttgtttcggcgtagaagtgtgtgtaacactggtcgttgcactgttaataagcatgtggcctatcagttatatatgattgatcctgcaggtgtct |
| | | gtcttggtgcaggtcaattcgtgggttgggtcatacccttagcctttatgcctgtgcaatcccggaaatttattgttccatgggttatgtacttgcgta |
| | | agcgtggcgaaaagggtgcttacaataaagatcatggacgtggcggttttggacatgtttatgattttaaagttgaagatgcttatgaccaggtgcatg |
| | | atgagcctaagggtaagttttctaagaaggcttatgattaattagagggtatcgtggtgttaaaccacttctctatgtagaccagtatggttgtgatta |
| | | tactggtagtcttgcagatggcttagaggcttatgctgataagacattgcaagaaatgaaggcattatttcctacttggagtcaggaactcctttttga |
| | | tgtaattgtggcatggcatgttgtgcgtgatccacgttatgttatgagattgcagagtgctgctactatacgtagtgttgcatatgttgctaatcctac |
| | | tgaagacttgtgtgatggttctgttgttataaaagaacctgtgcatgtttatgcagatgactctattattttacgtcaatataatttagttgacattat |
| | | gagtcatttttatatggaggcagatacagttgtaaatgctttttatggtgttgattgaaagattgcggttttgttatgcagtttggttacattgattgc |
| | | gaacaagactcgtgtgattttaaaggttggattcctggtaacatgatagatggttttgcttgcaccacttgtggtcatgtttatgaagtaggtgatttg |
| | | atggcacaatcttcaggtgttttgcctgttaaccctgtattgcatactaagagtgcagcaggctatggtggttttggttgtaaagattcttttactctg |
| | | tatggccaaactgtagtttattttggaggttgtgtgtattggagtccagcacgtaatatatggattcctatattaaaatcctctgttaagtcatatgac |
| | | agtttggtttatactggagttttaggttgcaaggctattgtaaaggaaacaaatctcatttgcaaagctttgtaccttgattatgttcaacacaagtgt |
| | | ggcaatttacaccaacgggagttgctaggtgtttcagatgtgtggcataaacaattgctattaaatagaggtgtttataaacctctgttagagaatatt |
| | | gattattttaatatgcggcgcgctaaatttagtttagaaacttttactgtttgtgcagatggctttatgccttttcttttagatgatttagttccacgc |
| | | gcatattatttggcagtaagtggtcaagcattttgtgattatgcagataaactttgccatgccgttgtgtctaagagtaaagagttacttgatgtgtct |
| | | ctggattctttaggtgcagctatacattatttgaattctaagattgttgatttggctcaacattttagtgattttggaacaagtttcgtttctaaaatt |
| | | gttcatttctttaagacttttactactagcactgctcttgcatttgcatgggttttatttcatgttttgcatggtgcttatatagtagtggagagtgat |
| | | atatattttgttaaaaacattcctcgttatgctagtgctgttgcacaagcatttcagagtgttgctaaagttgtactggactctttaagagttactttt |
| | | attgatggcctttcttgttttaagattggacgtagaagaatttgtctttcaggcagaaaaatttatgaagttgagcgtggcttgttacattcatcccaa |
| | | ttgccattagatgtttatgatttaaccatgcctagtcaagttcagaaagccaagcaaaaacctatttatttaaaaggttctggttctgatttttcatta |
| | | gcggatagtgtagttgaagttgttacaacttcacttacaccatgtggttattctgaaccacctaaagttgcagctaaaatttgcattgtggataatgtt |
| | | tatatggccaaggctggtgacaaatattaccctgttgtggttgatgatcatgttggactcttggatcaagcatggagagttccttgtgctggaaggcgt |
| | | gttacatttaaggaacagcctacagtaaaggagattataagcatgcctaagattattaaggttttttatgagcttgacaacgattttaatactatttta |
| | | aatactgcgtgtggagtgtttgaagtggatgatactgttgatatggaggaattttatgctgtggtgattgatgccatagaagagaaactttctccatgt |
| | | aaggagcttgaaggtgtaggtgctaaagttagtgcctttttacagaaattagaggataatcccctatttttatttgatgaggctggcgaggaagttctt |
| | | gctcctaaattgtattgtgcctttacagctcctgaagatgatgactttcttgaggaaagtgatgttgaagaagatgatgtagaaggtgaggaaactgat |
| | | ttaactgtcacaagtgctggacagccttgtgttgctagtgaacaggaggagtcttctgaagtcttagaggacactttggatgatggtccaagtgtggag |
| | | acatctgattcacaagttgaagaagatgtagaaatgtcggattttgttgatcttgaatctgtgattcaggattatgaaaatgtttgttttgagttttat |
| | | actacagagccagaatttgttaaagttttgggtctgtatgtgcctaaagcaactcgcaacaattgctggttgcgatcagttttggcagtgatgcagaaa |
| | | ttgccctgtcaatttaaagataaaaatttgcaggatctttgggtgttatacaagcaacagtatagtcagttgtttgttgataccttggttaataagata |
| | | cctgctaatattgtacttccacaaggtggttatgttgctgattttgcatattggtttttaaccttatgtgattggcagtgtgttgcatactggaaatgc |
| | | attaaatgtgatttagctcttaagcttaaaggcttggatgctatgttcttttatggtgatgttgtttcacatatatgcaagtgtggtgagtctatggta |
| | | cttattgatgttgatgtgccatttacagcccactttgctcttaaagataagttgttttgtgcatttattactaagcgtattgtgtataaagcagcttgt |
| | | gttgtggatgttaatgatagtcattctatggctgttgttgatggtaaacaaattgatgatcatcgtatcactagtattactagtgataagtttgatttt |
| | | attattgggcatggtatgtcattttcaatgactacttttgaaattgcccaattgtatggttcttgtataacacctaatgtgtgttttgttaaaggtgat |
| | | ataattaaagtatctaagcttgttaaagcagaagttgttgtaaaccctgctaatggccatatggcacatggtggtggtgttgcaaaagctattgcagta |
| | | gcagctggacagcagtttgttaaagagactaccgatatggttaagtctaaaggagtttgtgctactggagattgttatgtactacagggggcaaattat |
| | | gtaaaactgtgcttaatgttgttggacctgatgcgagaacacagggtaaacaaagttatgtattgttagagcgtgtttataaacatcttaacaactatg |
| | | actgtgttgttacaactttgatctcagctggtatatttagtgtgccttctgatgtgtctttaacatatctacttggtactgctaagaaacaagttgttc |
| | | ttgttagcaataatcaagaggattttgatcttatttctaagtgtcagataactgctgttgagggcactaagaaattggcagcgcgtctttatttaatgt |
| | | tggacgttccattgtttacgaaacagatgctaataagttgattttaatcaatgacgttgcatttgtttcgacatttaatgttttacaggatgttttatc |
| | | cttaagacatgatatagcacttgatgatgatgcacgaaccttcgttcagagcaatgttgatgttgtacctgagggttggcgtgttgtcaataagtttta |
| | | tcaaattaatggtgttagaaccgttaagtattttgagtgtactggaggcatagatatatgcagccaggataaagtttttggttatgtacagcagggtat |
| | | ttttaataaggctactgttgctcaaattaaagccttgtttttggataaagtggacatcttgctaactgttgatggtgttaatttcactaataggtttgt |
| | | gcctgttggtgaaagttttggtaagagtctaggaaatgtgttttgtgatggagttaatgtcacgaagcataagtgtgatataaattataaaggtaaagt |
| | | ctttttccagtttgataatctttctagtgaagatttaaaggctgtaagaagttcctttaattttgatcagaaggaattgcttgcctattacaacatgct |
| | | tgttaattgttttaagtggcaggttgttgttaatggtaagtatttcacttttaagcaagctaataacaattgttttgttaatgtttcttgcttaatgct |
| | | ccagagtttgcatctgacatttaaaattgttcaatggcaagaggcatggcttgaatttcgttctggccgccctgctagatttgtagctttggttttggc |
| | | caaaggtgggtttaaatttggagatcctgctgattctagagatttcttgcgtgttgtgtttagtcaagttgatttgactggggcaatatgtgattttga |
| | | aattgcatgtaaatgtggtgtaaagcaggaacagcgtactggtaggacgctgttatgcattttggtacattgagtcgtgaagatcttgagattggttat |
| | | accgtggactgttcttgcggtaaaaagctaattcattgtgtacgatttgatgtaccatttttaatttgcagtaatacacctgctagtgtaaaattacct |
| | | aagggtgtaggaagtgcaaatatttttataggtgataaggttggtcattatgttcatgttaagtgtgaacaatcttatcagctttatgatgcttctaat |
| | | gttaagaaggttacagatgttactggcaagttgtcagattgtctgtatcttaaaaatttgaaacaaacttttaaatcggtgttaaccacctattatttg |
| | | gatgatgttaagaaaattgagtataaacctgacttgtcacaatattattgtgacggaggtaagtattatactcagcgtattattaaagcccaatttaaa |
| | | acattcgagaaagtagatggtgtgtatactaattttaaattgataggacacaccgtagtgacagtcttaatgctaagttgggttttgatagctctaaag |
| | | agtttgttgaatataagattactgagtggccaacagctacaggtgatgtggtgttggctactgatgatttgtatgttaagagatatgagaggggttgta |
| | | ttactittggtaaacctgttatatggttaagccatgagaaagcttccctcaattattaacatattttaatagaccttcattggttgatgataataaatt |
| | | tgatgttttaaaagtggatgatgttgacgatggtggtgacagctcagagagtggtgccaaagaaaccaaagaaatcaacattattaagttaagtggtgt |
| | | taaaaaaccatttaaggttgaagatagtgtcattgttaatgatgatactagtgaaaccaaatatgttaagagtttgtctattgttgatgtgtatgatat |
| | | gtggcttacaggttgtaagtatgttgttagaactgctaatgattgagcagagcagttaacgtacctacaatacgtaagtttataaaatttggtatgact |
| | | cttgttagtataccaattgatttgttaaatttaagagagattaagcctgctgttaatgtggttaaagctgtgcgaaataaaatttctgtatgattaatt |
| | | ttattaaatggctttttgtcttattatttggctggattaaaatatccgctgataataaagtaatctacaccacagaaattgcatcaaagcttacgtgta |
| | | agcttgtagattagatttaaaaatgcatttttgacatttaagtggagtatggttgctagaggtgcttgcattatagcgactatatttctattgtggttt |
| | | aattttatatatgccaatgtaatttttagtgatttttatttgcctaaaatcggtttcttgccgacttttgttggtaagattgcacagtggattaagaac |
| | | acttttagtcttgtaactatttgtgatctatattccattcaggatgtgggttttaagaatcagtattgtaatggaagtattgcatgtcagttctgcttg |
| | | gcaggatttgatatgttagataattataaagccattgatgtagtacagtatgaagctgataggagagcatttgttgattatacaggtgtgttaaagatt |
| | | gtcattgaattgatagttagttacgccctgtatacggcatggttttatccattgtttgccdtatcagtattcagatcttgaccacttggctgcctgagc |
| | | tttttatgcttagtacattacattggagttttaggttgctggtggctttagctaatatgttaccagcacatgtgtttatgaggttttatattattattg |
| | | cctatttattaagctctttagcttgtttaggcatgttgcctatggttgtagtaaatctggttgtttgttttgttacaagaggaatcgtagtctacgtgt |
| | | taaatgtagtactatcgttggtggcatgatacgctattacgatgttatggctaatggtggcactggcttttgttcaaaacatcaatggaattgcattga |
| | | ttgtgattcttataaaccaggtaatacttttattactgttgaggccgctcttgatctatctaaggaattgaaacggcccattcagcctacagatgttgc |
| | | ttatcatacggttactgatgttaagcaagttggttgttctatgcgcttgttctatgatcgtgatggacagcgcacatatgatgatgttaatgctagttt |
| | | gtttgtggattatagtaatttgctacattctaaggttaagagtgtgcctaatatgcatgttgtggtagtggaaaatgatgctgataaagccaattttct |
| | | gaatgctgctgtattttatgcacagtctttgtttagacctattttaatggttgataaaaatctgataactactgctaacactggtacgtagttacagaa |
| | | actatgtttgatgtttatgtggatacatttttgtctatgtttgatgtggataaaaagagtcttaatgattaatagcaactgcgcattcttctataaaac |
| | | agggtacgcagatttataaagttttggatacctttttaagctgtgctcgtaaaagttgttctattgattcagatgttgatactaagtgtttagctgatt |
| | | ctgtcatgtctgctgtatcggcaggtcttgaattgacggatgaaagttgtaataacttggtgccaacatatttgaagagtgacaacattgtggcagctg |
| | | atttaggtgttctgattcaaaattctgcaaagcatgtgcagggtaatgttgctaaaatagctggtgtttcctgtatatggtctgtggatgcttttaatc |
| | | agtttagttctgatttccagcataaattgaagaaagcatgttgtaaaactggtttgaaactgaagcttacttataataagcagatggctaatgtactgt |
| | | tttaactacaccattagtcttaaagggggtgcagtttttagttattttgtttatgtgtgttttgtgttgagtttggtagttttattggactgtggtgct |
| | | taatgcccacttacacagtacacaaatcagattttcagcttcccgtttatgccagttataaagttttagataatggtgttattagagatgttagcgttg |
| | | aagatgtttgtttcgctaacaaatttgaacaatttgatcaatggtatgagtctacatttggtctaagttattatagtaacagtatggcttgtcccattg |
| | | ttgttgctgtaatagatcaggattttggctctacagtgtttaatgtccctaccaaagtgttacgatatggttatcatgtgttgcactttattacacatg |
| | | cactttctgctgatggagtgcagtgttatacgccacatagtcaaatatcgtattctaatttttatgctagtggctgtgtgattcctctgcttgcactat |
| | | gtttacaatggccgatggtagtccacaaccttattgttatacagaggggcttatgcaaaatgcttctctgtatagttcattggtacctcacgtgcggta |
| | | taatcttgctaatgctaaaggttttatccgttttccagaagtgttgcgagaagggcttgtacgtatcgtgcgtactcgttctatgtcgtattgcagagt |
| | | tggattatgtgaggaagctgatgagggtatatgctttaattttaatggttcttgggtgcttaataatgattattatagatcattgcctgggaccttttg |
| | | tggtagagatgtttttgatttaatttatcagctatttaaaggtttagcacagcctgtggattttttggcattgactgctagttccattgctggtgctat |
| | | actcgctgtaattgttgttttggtgttttattacctaataaagcttaaacgtgatttggtgattacaccagtgttgtttttgttaacgtgattgtgtgg |
| | | tgtgtaaattttatgatgctttttgtgtttcaagtttaccccatactttcttgtgtatatgctatttgttatttttatgccacgctttatttccdtcgg |
| | | agataagtgtgataatgcacttacaatggctagttatgtatggcactattatgcctttatggttttgtttgctatatatagctgttgttgtttcaaatc |
| | | atgctttttgggtattttcttactgcagaaagcttggtacttctgttcgtagtgatggtacatttgaagaaatggctctcactacttttatgattacaa |
| | | aagattcttattgtaagcttaagaattctttgtctgatgttgcttttaatagatatttgagtttgtataataaatataggtattacagcggtaaaatgg |
| | | atactgctgcatatagggaggctgcttgactcagttggctaaagcaatggacacatttaccaataataatggtagtgatgtgctttaccaaccgcctac |
| | | tgcttccgtctcaacttcattcttgcaatctggtattgtgaaaatggtaaatcctacttctaaggtagaaccatgtgttgtcagtgttacctatggtaa |
| | | tatgacattgaatggtttatggttggatgacaaggtctactgtcccagacatgtaatatgttctgcttcagatatgactaatccagattatacaaattt |
| | | gttgtgtagagtaacatcaagtgattttactgtattgtttgatcgtctaagccttacagtgatgtcttatcaaatgcggggttgtatgcttgttcttac |
| | | agtgaccctgcaaaattctcgtacgccaaaatatacatttggtgtggttaaacctggtgagacttttactgttttagctgcttataacggcaaaccaca |
| | | aggagcctttcatgtaactatgcgtagtagttataccattaagggttcdttttatgcggatcttgtggatctgttggttatgtaataatgggtgattgt |
| | | gttaaatttgtttatatgcatcaattggagcttagtactggttgtcatactggtactgacttcaatggggatttttatggtccttataaggatgctcag |
| | | gttgttcagttgctcattcaggattatatacaatctgttaattttgtagcatggctttatgctgctatacttaacaattgtaattggtttgtacaaagt |
| | | gataagtgttctgtagaagattttaatgtgtgggctctgtccaatggatttagccaagttaaatctgaccttgttatagatgattagcttctatgactg |
| | | gtgtgtattggaaacactgttggctgctattaagcgtcttaagaatggtttccaaggacgtcagattatgggtagttgctatttgaggatgaattgaca |
| | | cctagcgatgtttatcaacaactcgctggtatcaagttacaatcaaaacgcactagattgtttaaaggcactgtttgttggattatggcttctacattt |
| | | ttgtttagttgcataattacagcatttgtgaaatggactatgtttatgtatgtaactactaatatgtttagtattacgttttgtgcactttgtgttata |
| | | agtttggccatgttgttggttaagcataagcatctttatttgactatgtatataactcctgtgcttittacactgttgtataacaactatttggttgtg |
| | | tacaagcatacatttagaggctatgtctatgcatggctatcatattatgttccatcagttgagtacacttatactgatgaagttatttatggcatgtta |
| | | ttgcttgtaggaatggtattgttacattacgtagcattaaccatgatttgttttatttataatgtttgttggtcgtttgatttctgttttctattgtgg |
| | | tacaagggttctaacttagaggaagaaattcttcttatgttggcttccattttggtacttacacatggacaacagttttatctatggctgtagcaaagg |
| | | ttattgctaagtgggttgctgtgaatgtcttgtatttcacagatatacctcaaattaagatagtgatttgtgctatttgtttattggttatattattag |
| | | ctgttattggggcttgttttccttgatgaacagtttgtttagaatgcctttgggtgtttataattataaaatttcagtacaggaattaagatatatgaa |
| | | tgctaatggattgcgccctcctaagaatagttttgaagcccttatgcttaattttaagctgttgggtattggaggtgttccaatcattgaagtatctca |
| | | atttcaatcaaaattgactgatgtcaaatgtgctaatgtcgtcttgcttaattgcttgcaacatttgcatgttgcttctaattctaagttgtggcatta |
| | | ttgtagcactttgcacaatgaaatacttgccacttcggatctgagtgttgatttgaaaagcttgctcagttattaattgttttgtttgctaatccagct |
| | | gctgtggatagcaagtgcctgactagtattgaagaagtttgcgatgattacgcaaaggacaatactgttttgcaggctttacagagtgaatttgttaat |
| | | atggctagcttcgttgaatatgaagttgctaagaaaaatcttgatgaggcgcgttttagtggttctgctaatcaacagcagttaaaacagctagagaaa |
| | | gcctgtaatattgctaaatctgcttatgaacgcgaccgtgctgtagcaaaaaagttggagcgtatggctgatttggctctcactaatatgtataaagaa |
| | | gctagaattaatgataagaagagtaaggttgtttctgccttgcaaactatgctttttagtatggtgcgtaagttagataatcaagctctgaattcaata |
| | | ttagataacgctgtgaagggttgtgtaccattgaatgcaataccttcattggcagcaaatactctgaatataattgtaccagataaaagtgtttatgac |
| | | caggtagttgataatgtctatgttacctatgcgggtaatgtatggcagattcaaactatccaggattcagatggtacaaataagcagttgaatgagata |
| | | tctgatgattgtaactggccactagttattattgcaaatcggtataatgaggtatctgctactgttttgcaaaataatgaattaatgcctgctaagttg |
| | | aaaattcaggttgttaatagtggtccagatcagacttgtaatacacctactcaatgttactataataatagtaacaatgggaagattgtttatgctata |
| | | cttagtgatgttgatggtcttaagtatacaaaaattcttaaagatgatggcaattttgttgttttggagttagatcctccttgtaaatttactgttcaa |
| | | gatgctaaaggtcttaaaattaagtacctttattttgtaaaaggttgtaacacactagcaagaggctgggttgttggtacaatttcttctacagttaga |
| | | ttgcaagctggaactgctactgaatatgcttccaactcatctatattgtctttatgtgcgttttctgtagatcctaagaaaacgtatttagattttata |
| | | caacaaggaggaacacctattgccaattgtgttaaaatgttgtgtgaccatgctggtaccggtatggccattactgttaaacccgatgctaccactagt |
| | | caggattcatatggtggtgcgtctgtttgtatatattgccgcgcacgagttgaacacccagatgttgatgggttgtgcaaattacgcggcaagtttgta |
| | | caagtgcctgtaggtataaaagatcctgtgtcttatgttttgacacatgatgtttgtcgagtttgtggattttggcgggatggaagttgttcatgtgtt |
| | | agcactgacactactgttcaatcaaaagatactaattttttaaacgggttcggggtacgagtgtagatgcccgtctcgtaccctgcgccagtggtttat |
| | | ctactgatgtacaattaagggcatttgatatttacaatgctagtgttgctggcattggtttacatttaaaagttaattgttgccgttttcagcgtgttg |
| | | atgagaacggtgataaattagatcagttattgttgttaagaggacagatctgactatatataatagagagatgaaatgctatgagcgtgtaaaagattg |
| | | taagtttgtggctgaacacgatttattacatttgatgtagaaggtagtcgtgtgccacacattgtacgcaaggatttaacaaagtatactatgttggat |
| | | ctttgctatgcattgcgacattttgatcgcaatgattgcatgctgattgtgacattctctctatatatgctggttgtgaacaatcctactttactaaga |
| | | aggattggtatgattttgttgaaaatcctgatattattaatgtgtataaaaagctaggacctatttttaatagagccctagttagcgctactgagtttg |
| | | cggacaaattggtggaggtaggcttagtaggcgttttaacacttgataatcaagatttaaatggtaaatggtatgattttggtgactatgttattgcag |
| | | ccccaggatgtggtgttgctatagcagattcttattattcttatatcatgcctatgctgaccatgtgtcatgcattggattgcgaattgtatgtgaata |
| | | atgcttatagactatttgatcttgtacagtatgattttactgattacaagcttgaattgtttaataagtattttaagcactggagtatgccatatcatc |
| | | ctaacactgttgattgtcaggatgatcggtgtattatacattgtgctaattttaacatactttttagtatggttttacctaatacatgttttgggcctc |
| | | ttgttaggcaaatttttgtggatggtgtgccttttgttgtttcaattggctaccattataaagaacttggtattgtgatgaatatggatgtggatacac |
| | | atcgttatcgcttgtctttaaaagacttgcttttatatgctgctgatccagctttgcatgtagcttctgctagtgcattgtatgatttacgcacttgct |
| | | gttttagtgttgccgctataacaagcggtgtaaaatttcaaacagttaaacctggtaattttaatcaggatttttatgattttgttttaagtaaaggcc |
| | | tgcttaaagagggtagctcagttgatctgaagcactttttattacacaggatggtaatgctgctattactgattataattattataagtataatttgcc |
| | | caccatggtggacattaagcagttgttgtttgttttggaagttgtttataagtattttgagatttatgatggtgggtgtataccggcatcacaagtcat |
| | | tgttaataattatgataagagtgctggctatccatttaacaaatttggaaaagccaggctctattatgaagcattatcatttgaggaacaggatgaaat |
| | | ttacgcttatactaagcgtaatgtcctgccaacacttactcaaatgaatttgaaatatgctattagtgctaagaatagagcccgcactgttgctggtgt |
| | | ttccatacttagtactatgactggcagaatgtttcatcaaaaatgtttgaaaagtatagcagctacacgtggtgttcctgtagttataggcaccactaa |
| | | attttatggtggctgggatgatatgttacgccgccttattaaagatgttgacaatcctgtacttatgggttgggattatcctaagtgtgatcgtgctat |
| | | gccaaacctactacgtattgttagtagtttggtattagcccgaaaacatgagacatgttgttcgcaaagcgataggttttatcgacttgcgaatgaatg |
| | | cgcacaagttttgagtgaaattgttatgtgtggtggctgttattatgttaagcctggtggcactagtagtggtgatgcaactactgatttgctaattca |
| | | gtctttaacatatgtcaagctgtttcagccaatgtatgtgccttaatgtcatgcaatggcaataagattgaagatcttagtatacgtgctcttcagaag |
| | | cgcttatactcacatgtgtatagaagtgataaggttgattcaacctttgtcacagaatattatgaatttttaaataagcattttagtatgatgattttg |
| | | agtgatgatggggttgtgtgttataattctgattatgcgtccaaagggtatattgctaatataagtgcctttcaacaggtattatattatcaaaataac |
| | | gtttttatgtcagaatccaaatgttgggttgaacatgacataaataatggacctcatgaattctgttcacaacacacaatgcttgtaaagatggatggt |
| | | gacgatgtctaccttccatatcctaatcctagtcgtatattaggagaggatgttttgtagatgatttgttaaagactgatagtgttcttttaatagaac |
| | | gatttgtaagtcttgcaatagatgcttatccacttgtgtatcatgaaaatgaagaataccaaaaggtttttcgtgtttatttggcgtatataaagaagt |
| | | tgtacaatgacctgggtaatcagatcttggatagctacagtgttattttaagtacttgtgatggacaaaagttcactgatgagtccttttacaagaaca |
| | | tgtatttaagaagtgcagttatgcagagtgttggagcttgcgtggtctgctcttctcaaacatcattacgttgtggcagttgcatcagaaagcctcttc |
| | | tttgctgcaagtgttgttatgatcatgttatggcgactgatcataaatatgtcttgagtgtttcaccatatgtgtgtaatgcaccaggatgtgatgtaa |
| | | atgatgttaccaaattgtatctaggtggtatgtcatattattgtgaagaccataagccacaatattcattcaagttggtaatgaatggtaggtttttgg |
| | | tctatataaacaatcttgtacaggatctccgtacatagacgattttaatcgtatagctagttgtaaatggaccgatgtggatgattacatactagctaa |
| | | tgaatgtacagagcgcttgaaattgtttgctgcagaaacgcaaaaggcaaccgaggaagcctttaagcagagttatgcatcagcaacaatacaagagat |
| | | tgttagtgagcgcgaattgattctctcttgggagattggaaaagttaagccaccacttaataaaaattatgtttttactggctaccattttactaaaaa |
| | | tggtaagacagttttaggtgagtatgtttttgataagagtgagttgactaatggtgtgtattatcgcgccacaaccacttataagctatctgtaggaga |
| | | tgtttttgttttaacctctcattcagtagctaatttaagtgctcctacgcttgttccgcaggagaattatagtagtattagatttgctagtgtttatag |
| | | tgtgcttgagacgtttcagaacaatgttgttaattatcaacacattggtatgaaacgttactgcaccgtgcaaggacctcctggtacagggaagtcaca |
| | | tcttgctattggtcttgctgtattctattgtacagcacgtgttgtatacacagcggccagccatgcagctgttgacgcattgtgtgaaaaagcatataa |
| | | atttttgaatataaatgattgcactcgtattgttccggccaaggtcagggtggagtgctatgataagtttaaaattaatgacaccactcgtaagtatgt |
| | | gtttactaccataaatgcattacctgagatggtgactgatattgttgttgtagatgaagttagtatgcttaccaattatgagctttctgttattaatgc |
| | | tcgtattcgcgctaagcattatgtttatattggtgatcctgctcaattgccagcaccacgtgtgttattgagcaagggtacacttgaacctaaatattt |
| | | taacactgttactaagctcatgtgttgcttagggccagacattittcttggtacatgttatagatgtcctaaggaaatcgttgatacagtgtccgcctt |
| | | ggtttatgaaaataagcttaaggctaagaatgagagtagttcattgtgttttaaggtctattataagggcgttacaacacatgaaagttctagtgctgt |
| | | aaatatgcagcagatttatttgattaataagt-ttttgaaggctaaccattgtggcataaagctgtttttattagcccatataatagtcagaactttgc |
| | | agctaagcgtgttttgggtttacaaacccaaaccgtggattctgctcaaggttctgaatatgattatgttatatattcacagactgcagaaacagcgca |
| | | ttctgtaaatgttaatcgcttcaatgttgctattactcgagccaagaaaggtattctttgtgttatgagtaatatgcagttgtttgaagcattacagtt |
| | | tactacattgaccttagataaagtgccacaggccgtcgaaactaaagttcaatgtagtactaatttatttaaagattgtagcaagagttatagcggtta |
| | | tcacccagctcatgctccttcatttttggcagtagatgacaaatataaggcaactggcgatttagccgtgtgtcttggtattggtgattctgctgttac |
| | | atattcaagattaatatcactcatgggttttaaattggatgttacccttgatgggtattgtaagctttttataactaaagaagaagagttaaacgcgtg |
| | | cgtgcctgggttggctttgatgctgaaggtgctcatgccacgcgtgatagcattgggacaaatttcccacttcaattaggattttccacaggaattgat |
| | | tttgttgtggaagccactggtttgtttgctgatagagatggttacagctttaaaaaggctgtggcgaaagctcctcctggtgaacaatttaagcacctc |
| | | atccattgatgacgagaggtcatcgctgggatgttgttagacctagaatagtacaaatgtttgcagatcatttaattgatctgtctgattgtgttgtgc |
| | | tagttacatgggcagccaactttgagctcacttgtctccgctactttgcaaaagtagggcgtgagatttcttgtaatgtatgcactaaacgtgccacag |
| | | tttacaattctagaactggttactatggttgttggcgccatagtgttacatgtgattacttgtataatccacttattgttgatattcaacagtggggat |
| | | atattggttattatcaagtaatcatgatttatattgtagtgtccataaaggagcacatgttgcttcctctgatgctataatgacacggtgtttggccgt |
| | | ttatgattgcttttgcaataatattaattggaatgtggagtatcccatcatttcaaatgagttaagtattaatacctcttgtagggtcttgcagcgtgt |
| | | gattcttaaagctgccatgctctgcaacagatatactttgtgttatgatattggcaacccaaaagcgattgcctgtgtcaaagattttgattttaagtt |
| | | ctatgatgcccaaccaattgttaagtagttaagactcttttgtattatttgaggcacataaggactcttttaaagacggtttgtgtatgttttggaact |
| | | gtaatgtggataagtatccaccgaatgcagttgtatgtagatttgacactagagtgttgaataatttaaatcttcctggctgtaatggaggtagtttgt |
| | | atgttaataaacatgcattccacactaaaccattgctagggcagcctttgagcatttgaagcctatgccattcttctattattcagatacgccttgtgt |
| | | gtatatggatggcatggatgctaagcaggttgattatgtacctttgaaatctgccacgtgcatcacaagatgcaatttaggtggtgcagtttgtttaaa |
| | | acatgctgaagagtatcgtgagtacttagagtcttacaatacagctactacagcaggttttactttttgggtctataagacatttgatttttataattt |
| | | gtggaatacgttcaccaagctacaaagcttggagaatgttgtatataatttagtcaagactggtcattatacaggacaggctggtgaaatgccttgtgc |
| | | cattataaatgataaagttgtggctaagatcgataaggaggatgttgtcatttttattaataatacaacataccctactaatgtggccgttgaattatt |
| | | tgccaagcgcagtgttcgacaccacccagagcttaagctctttagaaatttaaatatagacgtgtgttggaagcacgtcatttgggattatgctagaga |
| | | aagtatattttgcagtaatacctatggtgtctgcatgtatacagatttaaagttcattgataaattgaatgtcctttttgatggtcgtgataatggtgc |
| | | tcttgaagcttttaaacgttctaataatggcgtttacatttccacgacaaaagttaagagtctttcgatgataagaggtccaccgcgtgctgaattaaa |
| | | tggcgtagtggtggacaaggttggagacactgattgtgtgttttattttgctgtgcgtaaagaaggtcaggatgtcatcttcagccaattcgacagcct |
| | | gggagtcagctctaaccagagcccacaaggtaatctggggagtaatggtaaacccggtaatgtcggtggtaatgatgctctgtcaatctctactatctt |
| | | tacacaaagccgtgttattagctcttttacatgtcgtactgatatggaaaaagattttatagctttagatcaagatgtgtttattcagaagtatggttt |
| | | ggaggactatgcctttgaacacattgtttatggtaacttcaaccagaagattattggtggtttgcatttgttaataggcttgtaccgaagacagcaaac |
| | | ttccaatctggttgttcaggagtttgtttcatatgactccagcatacactcttattttatcactgacgagaagagtggtggtagtaagagtgtttgcac |
| | | tgttatagatattttgttggatgattttgtggctcttgttaagtcacttaatcttaattgtgtgagtaaggttgttaatgttaatgttgattttaaaga |
| | | ttttcagtttatgattggtgtaacgatgagaaagttatgactttctatcctcgtttgcaagctgcatctgactggaagcctggttattctatgcctgta |
| | | ttatataagtatttgaattctccaatggaaagagttagtctctggaattatgggaagccagttactttgcctacaggctgtatgatgaatgttgctaag |
| | | tatactcagttatgtcaatatctgaatactacaacattagctgtacctgttaatatgcgagttttgcatttaggtgcaggttcagaaaaaggagtagca |
| | | ccgggttctgcagttcttaggcagtggttgcctgctggtactattcttgtagataacgatttatacccatttgttagtgacagtgtcgctacatatttt |
| | | ggggattgtataactttaccctttgattgtcaatgggatttgataatttctgatatgtatgaccctattactaagaacataggggagtacaatgtgagt |
| | | aaagatggtttattacatacatttgtcatatgattcgagacaagttagctctgggtggcagtgttgctataaaaataacagagttttcttggaatgcag |
| | | aattatataagttaatggggtattttgcattttggactgtgttttgcacaaatgcaaatgcttcttctagtgaaggatttttaattggcataaattatt |
| | | tgtgtaagcccaaggttgagatagatggaaatgttatgcatgccaattatttgttttggagaaattccacagtttggaacgggggtgcttatagcctgt |
| | | ttgatatggctaaattcccgcttaagttggctggtactgccgtaataaatttaagagcagaccagattaatgatatggtttattcccttcttgaaaagg |
| | | gtaaactacttattagagatacaaataaagaagttttcgttggtgacagtttggttaatgtaatctaaactttaaaaatggctgtcgcttatgcagaca |
| | | agcctaatcattttatcaattttccacttacccattttcagggttttgtgttaaattataaaggtttacaatttcaaattctcgatgaaggagtggatt |
| | | gtaaaatacaaacagcgccacacattagtcttactatgctggacatacagcctgaagactataaaagtgttgatgtcgctattcaagaagttattgatg |
| | | atatgcattggggtgatggttttcagattaaatttgagaatcctcacatcctaggaagatgcatagttttagatgttaaaggtgtagaagaattgcatg |
| | | acgatttagttaattacattcgtgataaaggttgtgttgctgaccaatccaggaaatggattggccattgcaccatagctcaactcacggatgcagcac |
| | | tgtccattaaggaaaatgttgattttataaacagcatgcaattcaattataaaatcaccatcaacccctcatcaccggctagacttgaaatagttaagc |
| | | tcggtgctgaaaagaaagatggtttttatgaaaccatagttagtcactggatgggaattcgttttgaatacacatcacccactgataagctagctatga |
| | | ttatgggttattgttgtttagatgtggtacgtaaagagctagaagaaggcgatcttcccgagaatgatgatgatgcttggtttaagctatcgtaccatt |
| | | atgaaaacaattcttggttcttccgacatgtctacaggaaaagttttcatttccgtaaggcttgtcaaaatttagattgtaattgtttggggttttatg |
| | | aatcttcagttgaagaatattaaactcagtgaaaatgtttttgcttcctagatttattctagttagctgcataattggtagcttaggtttttacaaccc |
| | | tcctaccaatgttgtttcgcatgtaaatggagattggtttttatttggtgacagtcgttcagattgtaatcatattgttaatatcaacccccataatta |
| | | ttcttatatggaccttaatcctgttctgtgtgattctggtaaaatatcatctaaagctggcaactccatttttaggagttttcactttaccgattttta |
| | | taattacacaggcgaaggtcaacaaattattttttatgagggtgttaattttacgccttatcatgcattaaatgcaaccgttctggtagtaatgatatt |
| | | tggatgcagaataaaggcttgttttatactcaggtttataagaatatggctgtgtatcgcagccttacttttgttaatgtaccatatgtttataatggc |
| | | tccgcacaagctacagctctttgtaaatctggtagtttagtccttaataaccctgcatatatagctcctcaagctaactaggggattattattataagg |
| | | ttgaagctgatttttatttgtcaggttgtgacgagtatatcgtaccactttgtatttttaacggcaagtttttgtcgaatacaaagtattatgatgata |
| | | gtcaatattattttaataaagacactggtgttatttatggtctcaattctacagaaaccattaccactggttttgatcttaattgttattatttagttt |
| | | taccctctggtaattatttagccatttcaaatgagctattgttaactgttcctacgaaagcaatctgtcttaataagcgtaaggattttacgcctgtac |
| | | aggttgttgattcgcggtggaacaatgccaggcagtctgataacatgacggcggttgcttgtcaacctccgtactgttattttcgtaattctactacca |
| | | actatgttggtgtttatgatattaatcatggagatgctggttttactagcatacttagtggtttgttatataattcaccttgtttttcgcagcaaggcg |
| | | tttttaggtatgataatgttagcagtgtctggcctctctacccctatggcagatgtcccactgctgctgatattaatatccctgatttacccatttgtg |
| | | tgtatgatccgctaccagttattttgcttggcattatttgggcgttgcgattgtaattattgtagttttgttgttatattttatggtggataatgttac |
| | | taggctgcatgatgcttagaccataatctaaacatgtttttgatacttttaatttccttaccaacggcttttgctgttataggagatttaaagtgtact |
| | | tcagataatattaatgataaagacaccggtcctcctcctataagtactgatactgttgatgttactaatggtttgggtacttattatgttttagatcgt |
| | | gtgtatttaaatactacgttgtttcttaatggttattaccctacttcaggttccacatatcgtaatatggcactgaagggaagtgtactattgagcaga |
| | | ctatggtttaaaccaccatttctttctgattttattaatggtatttttgctaaggtcaaaaataccaaggttattaaagatcgtgtaatgtatagtgag |
| | | ttccctgctataactataggtagtacttttgtaaatacatcctatagtgtggtagtacaaccacgtacaatcaattcaacacaggatggtgataataaa |
| | | ttacaaggtatttagaggtctctgtttgccagtataatatgtgcgagtacccacaaacgatttgtcatcctaacctgggtaatcatcgcaaagaactat |
| | | ggcatttggatacaggtgttgtttcctgtttatataagcgtaatttcacatatgatgtgaatgctgattatttgtattttcatttttatcaagaaggtg |
| | | gtactttttatgcatattttacagacactggtgttgttactaagtttttgtttaatgtttatttaggcatggcgctttcacactattatgtcatgcctc |
| | | tgacttgtaatagtaagcttactttagaatattgggttacacctctcacttctagacaatatttactcgctttcaatcaagatggtattatttttaatg |
| | | ctgttgattgtatgagtgattttatgagtgagattaagtgtaaaacacaatctatagcaccacctactggtgtttatgaattaaacggttacactgttc |
| | | agccaatcgcagatgtttaccgacgtaaacctaatcttcccaattgcaatatagaagcttggcttaatgataagtcggtgccctaccattaaattggga |
| | | acgtaagacattttcaaattgtaattttaatatgagcagcctgatgtcttttattcaggcagactcatttacttgtaataatattgatgctgctaagat |
| | | atatggtatgtgtttttccagcataactatagataagtttgctatacccaatggcaggaaggttgacctacaattgggtaatttgggctatttgcagtc |
| | | atttaactatagaattgatactactgcaacaagttgtcagttgtattataatttacctgctgctaatgtttctgttagcaggtttaatccttctacttg |
| | | gaataagagatttggttttatagaagattctgtttttaagcctcgacctgcaggtgttcttactaatcatgatgtagtttatgcacaacactgtttcaa |
| | | agctcctaaaaatttctgtccgtgtaaattgaatggttcgtgtgtaggtagtggtcctggtaaaaataatggtataggcacttgtcctgcaggtactaa |
| | | ttatttaacttgtgataatttgtgcactcctgatcctattacatttacaggtacttataagtgcccccaaactaaatctttagttggcataggtgagca |
| | | ctgttcgggtcttgctgttaaaagtgattattgtggaggcaattcttgtacttgccgaccacaagcatttttgggttggtctgcagactcttgtttaca |
| | | aggagacaagtgtaatatttttgctaattttattttgcatgatgttaatagtggtcttacttgttctactgatttacaaaaagctaacacagacataat |
| | | tcttggtgtttgtgttaattatgacctctatggtattttaggccaaggcatttttgttgaggttaatgcgacttattataatagttggcagaacctttt |
| | | atatgattctaatggtaatctctacggttttagagactacataacaaacagaacttttatgattcgtagttgctatagcggtcgtgtttctgcggcctt |
| | | tcacgctaactcttccgaaccagcattgctatttcggaatattaaatgcaactacgtttttaataatagtcttacacgacagctgcaacccattaacta |
| | | ttttgatagttatcttggttgtgttgtcaatgcttataatagtactgctatttctgttcaaacatgtgatctcacagtaggtagtggttactgtgtgga |
| | | ttactctaaaaacagacgaagtcgtggagcgattaccactggttatcggtttactaattttgagccatttactgttaattcagtaaacgatagtttaga |
| | | acctgtaggtggtttgtatgaaattcaaataccttcagagtttactataggtaatatggtggagtttattcaaacaagctctcctaaagttactattga |
| | | ttgtgctgcatttgtctgtggtgattatgcagcatgtaaatcacagttggttgaatatggtagtttctgtgataacattaatgccatactcacagaagt |
| | | aaatgaactacttgacactacacagttgcaagtagctaatagtttaatgaatggtgttactcttagcactaagcttaaagatggcgttaatttcaatgt |
| | | agacgacatcaatttttcccctgtattaggttgtctaggcagcgaatgtagtaaagcttccagtagatctgctatagaggatttactttttgataaagt |
| | | aaagttatctgatgtcggttttgttgaggcttataataattgtacaggaggtgccgaaattagggacctcatttgtgtgcaaagttataaaggcatcaa |
| | | agtgttgcctccactgctctcagaaaatcagatcagtggatacactttggctgccacctctgctagtctatttcctccttggacagcagcagcaggtgt |
| | | accattttatttaaatgttcagtatcgcattaatgggcttggtgtcaccatggatgtgctaagtcaaaatcaaaagcttattgctaatgcatttaacaa |
| | | tgccattatgctattcaggaagggttcgatgcaactaattctgattagttaaaattcaagctgttgttaatgcaaatgctgaagctcttaataacttat |
| | | tgcaacaactctctaatagatttggtgctataagtgcttctttacaagaaattctatctagacttgatgctcttgaagcggaagctcagatagatagac |
| | | ttattaatggtcgtcttaccgctcttaatgcttatgtttctcaacagcttagtgattctacactggtaaaatttagtgcagcacaagctatggagaagg |
| | | ttaatgaatgtgtcaaaagccaatcatctaggataaatttctgtggtaatggtaatcatattatatcattagtgcagaatgctccatatggtttgtatt |
| | | ttatccactttagttatgtccctactaagtatgtcacagcgagggttagtcctggtctgtgcattgctggtgatagaggtatagctcctaagagtggtt |
| | | attttgttaatgtaaataatacttggatgtacactggtagtggttactactaccctgaacctataactgaaaataatgttgttgttatgagtacctgcg |
| | | ctgttaattatactaaagcgccgtatgtaatgctgaacacttcaatacccaaccttcctgattttaaggaagagttggatcaatggtttaaaaatcaaa |
| | | catcagtggcaccagatttgtcacttgattatataaatgttacattcttggacctacaagttgaaatgaataggttacaggaggcaataaaagtcttaa |
| | | atcagagctacatcaatctcaaggacattggtacatatgaatattatgtaaaatggccttggtatgtatggcttttaatctgccttgctggtgtagcta |
| | | tgcttgttttactattcttcatatgctgttgtacaggatgtgggactagttgttttaagaaatgtggtggttgttgtgatgattatactggataccagg |
| | | agttagtaatcaaaacttcacatgacgactaagttcgtattgattcattgcactgatctcttgttagatctttttgcaatctagcatttgttaaagttc |
| | | ttaaggccacgccctattaatggacatttggagacctgagaagaaatatctccgttatattaacggttttaatgtctcagaattagaagatgcttgttt |
| | | taaatttaactatcaatttcctaaagtaggatattgtagagttcctagtcatgcttggtgccgtaatcaaggtagattttgtgctacattcactcttta |
| | | tggtaaatccaaacattatgataaatattttggagtaataaatggtttcacagcattcgctaatactgtagaggatgctgttaacaaactggttttctt |
| | | agctgttgactttattacctggcgcagacaggagttaaatgtttatggctgatgcttatcttgcagacactgtgtggtatgtggggcaaataattttta |
| | | tagttgccatttgtttattggttacaatagttgtagtggcatttttggcaacttttaaattgtgtattcaactttgcggtatgtgtaataccttagtac |
| | | tgtccccttctatttatgtgtttaatagaggtaggcagttttatgagttttacaatgatgtaaaaccaccagtccttgatgtggatgacgtttaggtaa |
| | | tccaaacattatgagtagtaaaactacaccagcaccagtttatatctggactgctgatgaagctattaaattcctaaaggaatggaatttttattgggt |
| | | attatactactttttattacaatcatattgcaatttggatatacaagtcgcagtatgtttgtttatgttattaagatgattattttgtggcttatgtgg |
| | | ccccttactataatcttaactattttcaattgcgtatacgcattgaataatgtgtatcttggcctttctatagtttttaccatagtggccattattatg |
| | | tggattgtgtattttgtgaatagtatcaggttgtttattagaactggaagtttttggagtttcaacccagaaacaaacaacttgatgtgtatagatatg |
| | | aaaggaacaatgtatgttaggccgataattgaggactatcatactctgacggtcacaataatacgcggccatattacattcaaggtataaaactaggta |
| | | ctggctattattggcagatttgccagcttatatgactgttgctaaggttacacacctgtgcacatataagcgtggttttcttgacaggataagcgatac |
| | | tagtggttttgctgtttatgttaagtccaaagtcggtaattaccgactgccatcaacccaaaagggttctggcatggacaccgcattgttgagaaataa |
| | | tatctaaattttaaggatgtcttttactcctggtaagcaatccagtagtagagcgtcctctggaaatcgttctggtaatggcatcctcaagtgggccga |
| | | tcagtccgaccagtttagaaatgttcaaaccaggggtagaagagctcaacccaagcaaactgctacctctcagcaaccatcaggagggaatgttgtacc |
| | | ctactattcttggttctctggaattactcagtttcaaaagggaaaggagtttgagtttgtagaaggacaaggtgtgcctattgcaccaggagtcccagc |
| | | tactgaagctaaggggtactggtacagacacaacagacgttatttaaaacagccgatggcaaccagcgtcaactgctgccacgatggtatttttactat |
| | | ctgggaacaggaccgcatgctaaagaccagtacggcaccgatattgacggagtctactgggtcgctagcaaccaggctgatgtcaataccccggctgac |
| | | attgtcgatcgggacccaagtagcgatgaggctattccgactaggtttccgcctggcacggtactccctcagggttactatattgaaggctcaggaagg |
| | | tctgctcctaattccagatctacttcgcgcacatccagcagagcctctagtgcaggatcgcgtagtagagccaattctggcaatagaacccctacctag |
| | | gtgtaacacctgacatggctgatcaaattgctagtcttgttctggcaaaacttggcaaggatgccactaaacctcagcaagtaactaagcatactgcca |
| | | aagaagtcagacagaaaattttgaataagccccgccagaagaggagccccaataaacaatgcactgttcagcagtgttttggtaagagaggccctaatc |
| | | agaattttggtggtggagaaatgttaaaacttggaactagtgacccacagttccccattcttgcagaactcgcacccacagaggtgcgtttttattgga |
| | | tcaagattagagttggccaaagtgcagaatttatctgggaatcctgacgagccccagaaggatgtttatgaattgcgctataacggcgcaattaggttt |
| | | gacagtacactttcaggttttgagaccataatgaaggtgctgaatgagaatttgaatgcctatcaacaacaagatggtatgatgaatatgagtccaaaa |
| | | ccacagcgtcagcgtggtcataagaatggacaaggagaaaatgataatataagtgttgcagtgcccaaaagccgcgtgcagcaaaataagagtagagag |
| | | ttgactgcagaggacatcagcatcttaagaagatggatgagccctatactgaagacacctcagaaatataagagaatgaaccttatgtcggcatctggt |
| | | ggtaacccctcgcagaaaagtcgagataaggcactctctatcagaatggatgtcttgctgctataatagatagagaaggttatagcagactatagatta |
| | | attagttgaaagttttgtgttgtaatgtatagtgttggagaaagtgaaagacttgcggaagtaattgccgacaagtgcccaagggaagagccagcatgt |
| | | taagttaccacccagtaattagtaaatgaatgaagttaattatggccaattggaagaatcacaaaaaaaaaaaaaaaaaaaaaaaaaaaa |

1. An antisense oligonucleotide (ASO) comprising a 5′-wing region (A′), a central region (B′), and a 3′-wing region (C′) that together comprise 8 to 22 total nucleotide units, wherein:
(a) a central region (B′) comprising 6 or more contiguous nucleotide units,
(b) a 5′-wing region (A′) comprising 2 to 6 locked nucleotide units, 2′ substituted nucleotide units, or a combination thereof, and
(c) a 3′-wing region (C′) comprising 2 to 6 locked nucleotide units, 2′ substituted nucleotide units, or a combination thereof; and
wherein the ASO is complementary or hybridizes to a viral target RNA sequence in a coronavirus.

2. The ASO of claim 1, wherein the 5′-wing region of the ASO comprises 2 to 6 phosphorothioate-linked locked nucleosides.

3. The ASO of claim 1, wherein the 3′-wing region of the ASO comprises 2 to 6 phosphorothioate-linked locked nucleosides.

4. The ASO of claim 1, wherein the central region of the ASO comprises at least 5 contiguous phosphorothioate-linked DNA nucleosides.

5. The ASO of claim 1, wherein the central region of the ASO comprises 8 to 10 contiguous phosphorothioate-linked DNA nucleosides.

6. The ASO of claim 2, wherein the locked nucleosides are selected from LNA, scpBNA, AmNA (N—H), AmNA (N-Me), GuNA, GuNA (N-R) where R is selected from Me, Et, i-Pr, t-Bu and combinations thereof.

7. The ASO of claim 1, wherein the 3′-wing region and the 5′-wing region comprise the same number of nucleotide units.

8. The ASO of claim 1, wherein the 3′-wing region and the 5′-wing region comprise 3 nucleotide units and the central region comprises 10 nucleotide units.

9. The ASO of claim 1, wherein the 3′-wing region and the 5′-wing region comprise 4 nucleotide units and the central region comprises 10 nucleotide units.

10. The ASO of claim 1, wherein the 3′-wing region and the 5′-wing region comprise a different number of nucleotide units.

11. The ASO of claim 1, wherein the 3′-wing region comprises 3 nucleotide units, the 5′-wing region comprises 5 nucleotide units, and the central region comprises 10 nucleotide units.

12. The ASO of claim 1 further comprising one or more conjugation moieties.

13. The ASO of claim 1, wherein one Tin the central region is replaced by (2s)T, one C in the central region is replaced by (5OH)C, one A is replaced by (8NH)A in the central region, and/or one G in the central region is replaced by (8NH)G.

14. The ASO of claim 1, wherein the 5′-wing region and/or the 3′-wing region comprises one or two ScpBNA, AmNA, or GuNA.

15. The ASO of claim 1, wherein the 5′-wing region and/or the 3′-wing region comprises a mA or mU appended to the end of the sequence.

16. The ASO of claim 15, wherein the mA or mU is linked to a conjugation moiety.

17. The ASO of claim 1, wherein the ASO comprises 14-22 nucleotide units.

18. The ASO of claim 1, wherein the viral target RNA sequence encodes a non-structural protein (nsp).

19. The ASO of claim 18, wherein the nsp is selected from the group consisting of nsp8, nsp9, nsp10, nsp11, nsp12, nsp13, nsp14, and nsp15.

20. The ASO of claim 1, wherein the ASO comprises at least 8 nucleotide units from any one of SEQ ID NOs: 1-5534.

21. The ASO of claim 1, wherein the ASO comprises a sequence of any one of SEQ ID NOs: 1-5534.

22. The ASO of claim 1, wherein the ASO is at least about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or about 100% identical or complementary to the viral target RNA sequence.

23. The ASO of claim 1, wherein the ASO comprises a sequence that is at least about 75%, about 76%, about 77%, about 78%, about 79%, 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or about 100% identical or complementary to at least 8 consecutive nucleotide units of GenBank Accession NC_045512.2.

24. The ASO of claim 23, wherein the ASO comprises a sequence that is at least 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or about 100% identical or complementary to at least 16 consecutive nucleotide units of GenBank Accession NC_045512.2.

25. The ASO of claim 1, wherein the ASO comprises a sequence that is at least 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or about 100% identical or complementary to at least 8 consecutive nucleotide units of any one of the sequences listed in Table 2.

26. The ASO of claim 25, wherein the ASO comprises a sequence that is at least 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or about 100% identical or complementary to at least 16 consecutive nucleotide units of any one of the sequences listed in Table 2.

27. An antisense oligonucleotide (ASO) comprising 14-22 nucleotide units, wherein the ASO comprises:
(a) a central region (B′) comprising 6 or more contiguous DNA nucleosides, wherein at least one of the contiguous DNA nucleosides is a modified nucleotide,
(b) a 5′-wing region (A′) comprising 2 to 6 locked nucleosides, 2′ substituted nucleosides, or a combination thereof, and
(c) a 3′-wing region (C′) comprising 2 to 6 locked nucleosides, 2′ substituted nucleosides, or a combination thereof,
wherein the ASO is complementary or hybridizes to a viral target RNA sequence in a coronavirus.

28. The ASO of claim 27, wherein the modified nucleotide occurs at the nucleotide at position 3 from the 5′ end of the central region.

29. The ASO of claim 27 or 28, wherein the modified nucleotide is a nucleotide having a protected or unprotected version of 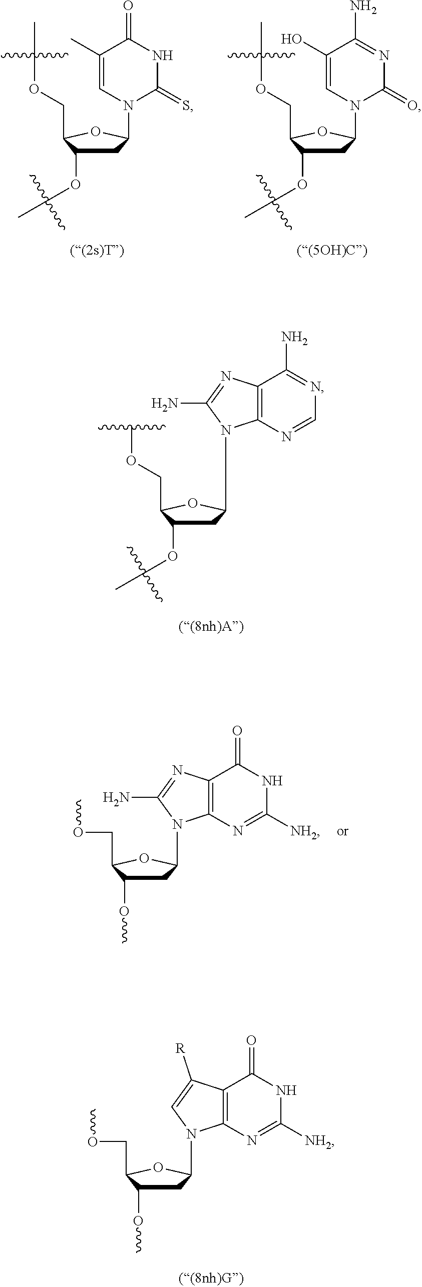
wherein:
R is a halogen or R′—C≡C—; and
R′ is C₆₋₁₂ aryl, 5- to 12-membered heteroaryl, hydroxy-C₁₋₆ alkyl, or C₁₋₇ alkanoyloxy.

30. The ASO of claim 27, wherein the modified nucleotide is a nucleotide having the structure of: 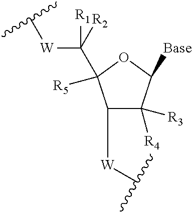
wherein:
W is independently O, N, or S;
R₁, R₂, and R₅ are independently H or D or CH₃ or CD₃;
R₃ is H or F;
R₄ is F or OCH₃ or OCD₃; and
Base is 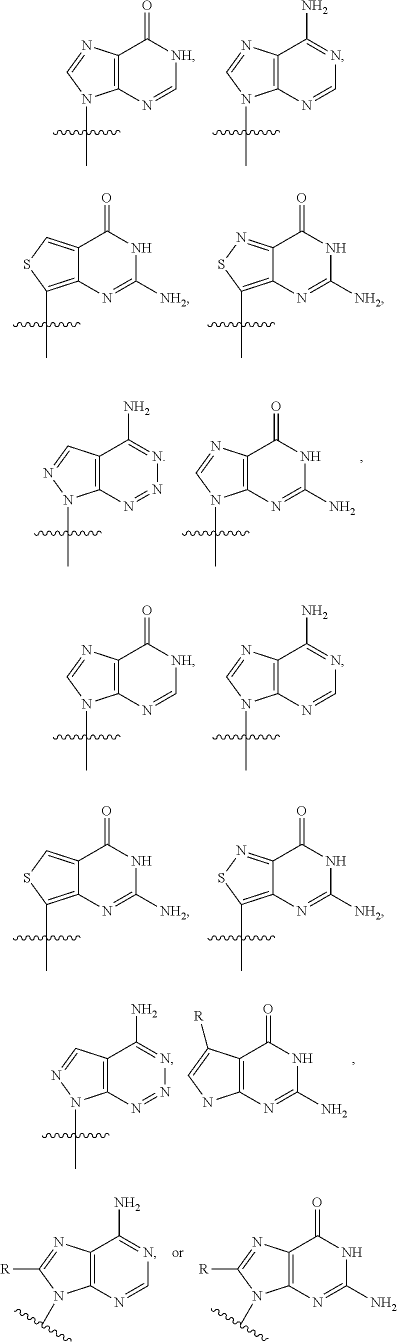
wherein:
R is a halogen or R′—C≡C—; and
R′ represents C₆₋₁₂ aryl, 5- to 12-membered heteroaryl, hydroxy-C₁₋₆ alkyl, or C₁₋₇ alkanoyloxy.

31. The ASO of claim 27, wherein the ASO comprises a nucleotide sequence that is at least 90% identical to a nucleotide sequence selected from the sequences listed in Table 1 or 2.

32. The ASO of claim 27, wherein the viral target RNA sequence encodes a non-structural protein (nsp).

33. The ASO of claim 32, wherein the nsp is selected from the group consisting of nsp8, nsp9, nsp10, nsp11, nsp12, nsp13, nsp14, and nsp15.

34. The ASO of claim 27, wherein the ASO is at least about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or about 100% identical or complementary to the viral target RNA sequence.

35. The ASO of claim 1, wherein the coronavirus is a β-coronavirus.

36. The ASO of claim 1, wherein the coronavirus is selected from the group consisting of CoV 229E, CoV NL63, CoV OC43, CoV HKU1, Middle East Respiratory Syndrome (MERS)-CoV, Severe Acute Respiratory Syndrome (SARS)-CoV, and SARS-CoV-2.

37. An antisense oligonucleotide (ASO) comprising any one of SEQ ID NOs: 5539-5718

38. The ASO of claim 37, wherein the ASO is selected from ASO-143 (SEQ ID NO: 5682), ASO-144 (SEQ ID NO: 5683), ASO-145 (SEQ ID NO: 5684), ASO-146 (SEQ ID NO: 5685), ASO-147 (SEQ ID NO: 5686), ASO-148 (SEQ ID NO: 5687), ASO-149 (SEQ ID NO: 5688), ASO-150 (SEQ ID NO: 5689), ASO-151 (SEQ ID NO: 5690), ASO-152 (SEQ ID NO: 5691), ASO-153 (SEQ ID NO: 5692), ASO-154 (SEQ ID NO: 5693), ASO-155 (SEQ ID NO: 5694), ASO-156 (SEQ ID NO: 5695), ASO-158 (SEQ ID NO: 5697), ASO-159 (SEQ ID NO: 5698), ASO-160 (SEQ ID NO: 5699), ASO-163 (SEQ ID NO: 5702), ASO-164 (SEQ ID NO: 5703), ASO-165 (SEQ ID NO: 5704), ASO-166 (SEQ ID NO: 5705), ASO-167 (SEQ ID NO: 5706), ASO-168 (SEQ ID NO: 5707), ASO-169 (SEQ ID NO: 5708), ASO-170 (SEQ ID NO: 5709), ASO-171 (SEQ ID NO: 5710), ASO-172 (SEQ ID NO: 5711), ASO-173 (SEQ ID NO: 5712), ASO-174 (SEQ ID NO: 5713), ASO-175 (SEQ ID NO: 5714), ASO-176 (SEQ ID NO: 5715), ASO-177 (SEQ ID NO: 5716), ASO-178 (SEQ ID NO: 5717), and ASO-179 (SEQ ID NO: 5718).

39-40. (canceled)

41. A pharmaceutical composition comprising one or more of the ASOs of claim 1 and a pharmaceutically acceptable carrier.

42. The pharmaceutical composition of claim 41, wherein the composition is formulated for intravenous (IV), subcutaneous, or inhalation administration.

43-44. (canceled)

45. A method of treating or preventing a disease caused by a β-coronavirus, comprising administering to a subject in need thereof a therapeutically effective amount of one or more of the ASOs of claim 1.

46-51. (canceled)

52. The method of claim 45, wherein the disease a β-coronavirus is a coronavirus infection selected from the group consisting of Middle East Respiratory Syndrome (MERS), Severe Acute Respiratory Syndrome (SARS), and COVID-19.

53. The method of claim 45, wherein the subject has been treated with one or more additional coronavirus treatment agents and/or antiviral agents.

54. The method of claim 45, wherein the subject is concurrently treated with one or more additional coronavirus treatment agents and/or antiviral agents.

55. The method of claim 45, wherein the subject is a human.

56. The method of claim 55, wherein the subject is 60 years old or older.

57-58. (canceled)

59. The method of claim 45, wherein the ASO is administered intravenously, subcutaneously, or via inhalation.

60-61. (canceled)
